(19) United States
(12) Patent Application Publication
(10) Pub. No.: US 2023/0293519 A1
(43) Pub. Date: Sep. 21, 2023

(54) INTEGRIN INHIBITORS AND USES THEREOF IN COMBINATION WITH OTHER AGENTS

(71) Applicant: Pliant Therapeutics, Inc., South San Francisco, CA (US)

(72) Inventors: Eric LEFEBVRE, San Francisco, CA (US); Gregory P. COSGROVE, Centennial, CO (US)

(21) Appl. No.: 17/966,717

(22) Filed: Oct. 14, 2022

Related U.S. Application Data
(60) Provisional application No. 63/359,875, filed on Jul. 10, 2022.
(60) Provisional application No. 63/359,835, filed on Jul. 9, 2022.
(60) Provisional application No. 63/255,898, filed on Oct. 14, 2021.

(51) Int. Cl. A61K 31/506; A61K 31/517; A61K 31/519; A61K 31/501; A61K 31/502; A61K 31/4375; A61P 11/00

(52) U.S. Cl. CPC A61K 31/506; A61K 31/4375; A61K 31/501; A61K 31/502; A61K 31/517; A61K 31/519; A61P 11/00

(57) ABSTRACT

The invention relates to methods of treating a subject for a disease comprising administration of compounds of Formula (A), Formula (I), or Formula (II):

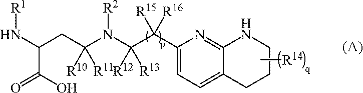

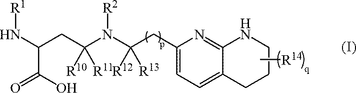

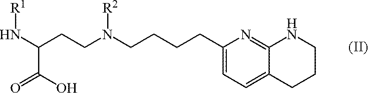

or a salt thereof, wherein R¹, R², R¹⁰, R¹¹, R¹², R¹³, R¹⁴, R¹⁵, R¹⁶, q and p are as described herein; and administering to the subject at least a second drug selected from pirfenidone and nintedanib, or a salt thereof. Compounds of Formula (A), Formula (I), Formula (II), and pharmaceutical compositions thereof are αvβ₆ integrin inhibitors that are useful for treating fibrosis such as idiopathic pulmonary fibrosis (IPF) and nonspecific interstitial pneumonia (NSIP).

### CROSS REFERENCE TO RELATED APPLICATIONS

This patent application claims priority benefit of U.S. Provisional Pat. Application No. 63/255,898, filed Oct. 14, 2021, of U.S. Provisional Pat. Application No. 63/359,835, filed Jul. 9, 2022, and of U.S. Provisional Pat. Application No. 63/359,875, filed Jul. 10, 2022. The entire contents of those patent applications are hereby incorporated by reference herein.

### BACKGROUND OF THE INVENTION

Fibrosis, a pathologic feature of many diseases, is caused by a dysfunction in the body’s natural ability to repair damaged tissues. If left untreated, fibrosis can result in scarring of vital organs causing irreparable damage and eventual organ failure.

Patients with nonalcoholic fatty liver disease (NAFLD) may progress from simple steatosis to nonalcoholic steatohepatitis (NASH) and then fibrosis. While liver fibrosis is reversible in its initial stages, progressive liver fibrosis can lead to cirrhosis.

Fibrosis in the kidney, characterized by glomerulosclerosis and tubulointerstitial fibrosis, is the final common manifestation of a wide variety of chronic kidney diseases (CKD). Irrespective of the initial causes, progressive CKD often results in widespread tissue scarring that leads to destruction of kidney parenchyma and end-stage renal failure, a devastating condition that requires dialysis or kidney replacement.

Scleroderma encompasses a spectrum of complex and variable conditions primarily characterized by fibrosis, vascular alterations, and autoimmunity. The scleroderma spectrum of disorders share the common feature of fibrosis, resulting in hardening or thickening of the skin. For some patients, this hardening occurs only in limited areas, but for others, it can spread to other major organs.

Following myocardial infarction, cardiac structural remodeling is associated with an inflammatory reaction, resulting in scar formation at the site of the infarction. This scar formation is a result of fibrotic tissue deposition which may lead to reduced cardiac function and disruption of electrical activity within the heart.

Crohn’s Disease is a chronic disease of unknown etiology tending to progress even in the setting of medical or surgical treatment. Intestinal fibrosis is among the most common complications of Crohn’s disease, resulting in stricture formation in the small intestine and colon.

Idiopathic pulmonary fibrosis (IPF) is a chronic, progressive, fibrosing disease of unknown etiology, occurring in adults and limited to the lungs. In IPF, the lung tissue becomes thickened, stiff, and scarred. As lung fibrosis progresses, it becomes more difficult for the lungs to transfer oxygen into the bloodstream and the organs do not receive the oxygen needed to function properly. IPF currently affects approximately 200,000 people in the U.S., resulting in 40,000 deaths per year. Patients diagnosed with IPF experience progressive breathlessness and eventually, complete respiratory failure.

Primary biliary cholangitis (PBC), also known as primary biliary cirrhosis, is a chronic disease of the liver that causes damage and fibrosis in the liver. It results from a slow, progressive destruction of the small bile ducts of the liver, causing bile and other toxins to build up in the liver, a condition called cholestasis. Over time, this leads to scarring and fibrosis in both the liver and biliary tract.

Nonspecific interstitial pneumonia (NSIP) is a rare disorder that affects the tissue that surrounds and separates the tiny air sacs of the lungs. These air sacs, called the alveoli, are where the exchange of oxygen and carbon dioxide takes place between the lungs and the bloodstream. Interstitial pneumonia is a disease in which the mesh-like walls of the alveoli become inflamed. The pleura (a thin covering that protects and cushions the lungs and the individual lobes of the lungs) might become inflamed as well. There are two primary forms of NSIP - cellular and fibrotic. The cellular form is defined mainly by inflammation of the cells of the interstitium. The fibrotic form is defined by thickening and scarring of lung tissue. This scarring is known as fibrosis and is irreversible. When the lung tissue thickens or becomes scarred, it does not function as effectively. Breathing becomes less efficient, and there are lower levels of oxygen in the blood. (Kim et al., Proc. Am. Thorac. Soc. (2006) 3:285-292; Lynch, D., Radiology (2001) 221:583-584; Kinder et al., Am. J. Respir. Crit. Care Med. (2007) 176:691-697)

Available courses of treatment are scarce, as there are currently no options on the market proven to have an effect on long-term patient survival or symptomatology. For example, agents such as pirfenidone and nintedanib have been studied for treatment of fibrosis. In the treatment of IPF, pirfenidone and nintedanib have been used, but have shown less therapeutic efficacy than desired while also exhibiting numerous side effects. There remains a need for treatment of fibrotic diseases.

The αvβ₆ integrin is expressed in epithelial cells, and binds to the latency-associated peptide of transforming growth factor-β1 (TGFβ1) and mediates TGFβ1 activation. Its expression level is significantly increased after injury to lung and cholangiocytes, and plays a critical in vivo role in tissue fibrosis. Increased levels are also associated with increased mortality in IPF and NSIP patients.

Primary sclerosing cholangitis (PSC) involves bile duct inflammation, and fibrosis that obliterates the bile ducts. The resulting impediment to the flow of bile to the intestines can lead to cirrhosis of the liver and subsequent complications such as liver failure and liver cancer. Expression of avβ₆ is elevated in liver and bile duct of PSC patients.

The present disclosure provides for αvβ₆ integrin inhibitors that may be useful for treatment of fibrosis.

### BRIEF SUMMARY OF THE INVENTION

Disclosed are amino acid compounds that are avβ₆ integrin inhibitors, compositions containing these compounds and methods for treating diseases mediated by αvβ₆ integrin such as a fibrotic disease.

In one aspect, provided is a compound of formula (A), or any variation thereof, or a salt thereof (e.g., a pharmaceutically acceptable salt thereof), as detailed herein.

Further provided is a pharmaceutical composition comprising a compound of formula (A), or any variation thereof detailed herein, or a salt thereof (e.g., a pharmaceutically acceptable salt thereof), and a pharmaceutically acceptable carrier or excipient.

In another aspect, provided is a method of treating a fibrotic disease in an individual (such as a human) in need thereof comprising administering to the individual a therapeutically effective amount of a compound of formula (A), or any variation thereof detailed herein, or a pharmaceutically acceptable salt thereof. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC). In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is pulmonary fibrosis (such as IPF). In some embodiments, the fibrotic disease is liver fibrosis. In some embodiments, the fibrotic disease is skin fibrosis. In some embodiments, the fibrotic disease is psoriasis. In some embodiments, the fibrotic disease is scleroderma. In some embodiments, the fibrotic disease is cardiac fibrosis. In some embodiments, the fibrotic disease is renal fibrosis. In some embodiments, the fibrotic disease is gastrointestinal fibrosis. In some embodiments, the fibrotic disease is primary sclerosing cholangitis. In some embodiments, the fibrotic disease is biliary fibrosis (such as PBC).

In another aspect, provided is a method of delaying the onset and/or development of a fibrotic disease in an individual (such as a human) who is at risk for developing a fibrotic disease comprising administering to the individual a therapeutically effective amount of a compound of formula (A), or any variation thereof detailed herein, or a pharmaceutically acceptable salt thereof. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or PBC. In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is psoriasis. In some embodiments, the individual at risk of developing a fibrotic disease has or is suspected of having NAFLD, NASH, CKD, scleroderma, Crohn’s Disease, NSIP, PSC, PBC, or is an individual who has had or is suspected of having had a myocardial infarction. In some embodiments, the individual at risk of developing a fibrotic disease has or is suspected of having psoriasis.

Also provided is a compound of formula (A), or any variation thereof detailed herein, or a pharmaceutical composition thereof, for the treatment of a fibrotic disease.

Also provided is use of a compound of formula (A), or any variation thereof detailed herein, or a pharmaceutically acceptable salt thereof, or a pharmaceutical composition comprising any of the foregoing, in the manufacture of a medicament for the treatment of a fibrotic disease.

Further provided is a kit comprising a compound of formula (A), or any variation thereof detailed herein, or a pharmaceutically acceptable salt thereof. In some embodiments, the kit comprises instructions for use according to a method described herein, such as a method of treating a fibrotic disease in an individual.

In another aspect, provided is a method of making a compound of formula (A) or any variation thereof, or a pharmaceutically acceptable salt thereof. Also provided are compound intermediates useful in synthesis of a compound of formula (A), or any variation thereof.

In one aspect, provided is a compound of formula (I), or any variation thereof, or a salt thereof (e.g., a pharmaceutically acceptable salt thereof), as detailed herein.

Further provided is a pharmaceutical composition comprising a compound of formula (I), or any variation thereof detailed herein, or a salt thereof (e.g., a pharmaceutically acceptable salt thereof), and a pharmaceutically acceptable carrier or excipient.

In another aspect, provided is a method of treating a fibrotic disease in an individual (such as a human) in need thereof comprising administering to the individual a therapeutically effective amount of a compound of formula (I), or any variation thereof detailed herein, or a pharmaceutically acceptable salt thereof. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC). In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is psoriasis.

In another aspect, provided is a method of delaying the onset and/or development of a fibrotic disease in an individual (such as a human) who is at risk for developing a fibrotic disease comprising administering to the individual a therapeutically effective amount of a compound of formula (I), or any variation thereof detailed herein, or a pharmaceutically acceptable salt thereof. In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or PBC. In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is psoriasis. In some embodiments, the individual at risk of developing a fibrotic disease has or is suspected of having NAFLD, NASH, CKD, scleroderma, Crohn’s Disease, NSIP, PSC, PBC, or is an individual who has had or is suspected of having had a myocardial infarction. In some embodiments, the individual at risk of developing a fibrotic disease has or is suspected of having psoriasis.

Also provided is a compound of formula (I), or any variation thereof detailed herein, or a pharmaceutical composition thereof, for the treatment of a fibrotic disease.

Also provided is use of a compound of formula (I), or any variation thereof detailed herein, or a pharmaceutically acceptable salt thereof, or a pharmaceutical composition comprising any of the foregoing, in the manufacture of a medicament for the treatment of a fibrotic disease.

Further provided is a kit comprising a compound of formula (I), or any variation thereof detailed herein, or a pharmaceutically acceptable salt thereof. In some embodiments, the kit comprises instructions for use according to a method described herein, such as a method of treating a fibrotic disease in an individual.

In another aspect, provided is a method of making a compound of formula (I) or any variation thereof, or a pharmaceutically acceptable salt thereof. Also provided are compound intermediates useful in synthesis of a compound of formula (I), or any variation thereof.

In another aspect, provided is a method of treating a subject for a disease, comprising: administering to the subject a first drug comprising a compound of formula (A) or a salt thereof; and administering to the subject at least a second drug that is selected from the group consisting of pirfenidone and nintedanib, or a salt thereof, whereby the subject is treated for the disease.

In any of the embodiments disclosed herein, the compound for use in any of the methods, including methods of treatment of disease or methods of treating a subject in need thereof, can be a compound, salt, or polymorph disclosed in International Patent Application No. WO 2022/109598 or U.S. Pat. Application Publication No. US 2022/0177468.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows compounds 1-780 as disclosed herein.

FIG. 2 shows Table B-3, with biological data for various compounds disclosed herein.

FIG. 3A is a graph showing that compound 5 and the selective antibody αvβ₆ inhibitor 3G9 both substantially inhibited normal bronchial epithelial cell adhesion to LAP, in contrast with the αvβ₁-selective small molecule inhibitor.

FIG. 3B shows that compound 5 and the αvβ₁-selective small molecule inhibitor both substantially inhibited cell adhesion in the IPF-derived lung fibroblasts, in contrast to the selective antibody avβ₆ inhibitor, 3G9.

FIG. 4A is a graph of PSMAD3/SMAD3 in lung tissue from healthy mice administered PBS vehicle and varying levels of compound 5 for 4 days.

FIG. 4B is a graph of PSMAD3/SMAD3 in BALF drawn from the same healthy mice administered PBS vehicle and varying levels of compound 5 for 4 days.

FIG. 4C is a graph showing that compared to the healthy mice, lung tissue in the vehicle-treated mice experienced a substantial increase in SMAD3 phosphorylation.

FIG. 4D is a graph showing that compared to the healthy mice, lung tissue in the vehicle-treated mice experienced a substantial accumulation of new collagen as evidenced by the percentage of lung collagen containing ²H-labeled hydroxyproline.

FIG. 4E shows that compared to the healthy mice, the vehicle-treated mice experienced a significant increase in total pulmonary collagen, as measured by µg of hydroxyproline.

FIG. 4F is a high resolution second harmonic generation image of fibrillar collagen (collagen type I and III) taken from formalin-fixed paraffin embedded lung tissue sections from a healthy mouse lung.

FIG. 4G is a high resolution second harmonic generation image of fibrillar collagen (collagen type I and III) taken from formalin-fixed paraffin embedded lung tissue sections from a vehicle-treated mouse lung.

FIG. 4H is a high resolution second harmonic generation image of fibrillar collagen (collagen type I and III) taken from formalin-fixed paraffin embedded lung tissue sections from a test-article treated mouse lung (500 mg/kg BID of compound 5).

FIG. 4I is a graph showing the percent total collagen area in the second harmonic generation mouse lung images of FIGS. 4F, 4G, and 4H.

FIG. 4J is a graph of sequential measurements in bleomycin-treated mice, which demonstrated a close inverse relationship between pSMAD3 levels in lung vs. plasma drug exposure.

FIG. 4K is a graph of sequential measurements in bleomycin-treated mice, which demonstrated a close inverse relationship between pSMAD3 levels in BALF cells vs. plasma drug exposure.

FIG. 5A is a bar graph, normalized to control slices treated with DMSO, showing that all test treatments reduced Type I Collagen gene Col1a1 expression.

FIG. 5B is a bar graph, normalized to control slices treated with DMSO, showing that all test treatments reduced lung Col1a1 expression.

FIG. 6A is a bar graph showing that compared to the DMSO vehicle control slices, both nintedanib and pirfenidone showed a slight increase in lung Col1a1 expression.

FIG. 6B is a bar graph showing the concentration of compound needed to reduce lung slice Col1a1 expression by 50% compared to DMSO control slices.

FIG. 6C is a bar graph, normalized to control slices treated with DMSO, showing that all test treatments reduced lung Col1a1 expression.

FIG. 6D is a bar graph showing relative expression of COL1A1 in precision cut lung slices (PCLS) from idopathic pulmonary fibrosis (IPF) lung tissue upon exposure to Comopund 5, clinical standard of care compounds nintedanib (Nin) and pirfenidone (Pirf), and an ALK5 inhibitor, all versus DMSO control.

FIG. 6E is a bar graph showing a dose dependent reduction of COL1A1 expression in PCLS from human IPF lung tissue upon treatment with concentrations of compound 5 ranging from 200 pM to 1 µM. COL1A1 expression is also graphed for the PCLS in the presence of 0.1% DMSO control, and an Alk5 inhibitor at 1 µM.

FIG. 6F is a bar graph showing the effect of dual selective αvβ₆ and αvβ₁ inhibition (Compound 5 at 1.82 µM) on the ratio of pSMAD2/SMAD2 in PCLS from human IPF lung tissue samples. The ratio of pSMAD2/SMAD2 is also graphed for the PCLS in the presence of 0.1% DMSO control, and an Alk5 inhibitor at 1 µM

FIG. 7A shows single ascending dose (SAD) study data for administration of 15, 30, 50, and 75 mg of Compound 5.

FIG. 7B shows the multiple ascending dose (MAD) study data for administration of 10, 20, and 40 mg of Compound 5.

FIGS. 8A-8F are a series of graphs showing data for subjects administered 40 mg/day of the selected integrin inhibitor (compound 5). The data in FIGS. 8A-8F include the blood plasma concentration (“PK”, round dots) of the administered integrin inhibitor and the relative change in pSMAD2:SMAD2 ratio from baseline (Day -1) in BAL (bronchoalveolar lavage) samples (“pSMAD”, square dots) through the displayed time course (hours) subsequent to the dose of inhibitor administered on Day 7. The peak of the blood plasma concentration (“PK” curve) is recorded as Cₘₐₓ.

FIG. 8G shows the % change in BAL SMAD2 phosphorylation levels (pSMAD2:SMAD2 ratio) on Day 7 compared to baseline levels recorded on Day -1, for subjects receiving placebo treatment, and subjects in which the Cₘₐₓ of the integrin inhibitor was measured to be less than 700 ng/mL, from 700 ng/mL to 900 ng/mL, and greater than 900 ng/mL.

FIG. 8H shows the % change in SMAD2 phosphorylation (pSMAD2:SMAD2 ratio) (all timepoints) correlated with Cₘₐₓ in subjects administered a 40 mg dose of Compound 5) compared to baseline levels recorded on Day -1.

FIG. 9 is a graph of unbound plasma concentration (X-axis) vs Vt (Y-axis) for the baseline Vt at each dose, the measured Vt after each dose, and a fit line.

FIG. 10 is a graph of unbound plasma concentration (X-axis) vs % receptor occupancy (Y-axis).

FIG. 11 is a bar chart showing % target engagement for each subject and dose.

FIG. 12 describes dose dependent effects of Compound 5.

FIG. 13 describes study design and objectives.

FIG. 14 is a summary of results of the Compound 5 study.

FIG. 15 describes study participant disposition.

FIG. 16 describes baseline demographics of the study participants.

FIG. 17 describes baseline disease characteristics of the study participants.

FIG. 18 summarizes overall safety of the study.

FIG. 19 summarizes overall safety of the study with and without background Standard of Care (SoC).

FIG. 20 describes the most frequent treatment emergent adverse events (TEAEs).

FIG. 21 illustrates that no treatment-emergent serious adverse events (SAEs) were observed with Compound 5.

FIG. 22 provides an overall summary of safety evaluation.

FIG. 23 provides an overall summary of pharmacokinetics.

FIG. 24 illustrates the change in FVC (forced vital capacity) from baseline to Week 12.

FIG. 25 illustrates the change in FVC over time in pooled Compound 5 groups.

FIG. 26 illustrates the change in FVC over time in the 40 mg Compound 5 group.

FIG. 27 illustrates the change in FVC over time in the 80 mg Compound 5 group.

FIG. 28 illustrates the change in FVC over time in the 160 mg Compound 5 group.

FIG. 29 illustrates the change in FVC from baseline to Week 12 in the subgroup on Standard of Care.

FIG. 30 illustrates the change in FVC from baseline to Week 12 in the subgroup not on Standard of Care.

FIG. 31 illustrates the proportion of participants with forced vital capacity-% predicted (FVCpp) decline greater than or equal to 10%.

FIG. 32 provides an overall summary of spirometry evaluation.

FIG. 33 compares serum biomarkers of collagen synthesis in Compound 5 groups versus placebo groups.

FIG. 34 describes study conclusions and next steps.

FIG. 35 illustrates the Mean Percent Change in Quantitative Lung Fibrosis (the extent from baseline to week 12 in the CT Protocol Population), using High-Resolution Computed Tomography (HRCT) based Quantitative Lung Fibrosis (QLF) imaging.

FIG. 36 illustrates the Mean Percent Change in Quantitative Lung Fibrosis (the extent from baseline to week 12) in the CT Protocol Population within Screening Window, using High-Resolution Computed Tomography (HRCT) based Quantitative Lung Fibrosis (QLF) imaging.

FIG. 37 illustrates overlap of genes significantly altered (adj. p<0.05, | log2FC | >0.5) by Compound 5 alone, and Compound 5 in combination with nintedanib or pirfenidone. Regions A through G of the Venn diagrams, as indicated in the legend, show the number of genes as follows, where Compound X is either pirfenidone or nintedanib: A. Genes only significanty changed by combination significantly of Compound 5 + Compound X; B: Genes significantly altered by Compound 5 alone and in combination; C. Genes significantly altered by Compound X alone and in combination; D. Genes significantly altered by Compound 5 and Compound X alone and in combination; E. Genes significantly altered by Compound 5 only in the absence of Compound X; F: Genes significantly altered by Compound 5 and Compound X alone, but not in combination; G. Genes significantly altered by Compound X only in the absence of Compound 5.

FIG. 38 illustrates the log2 fold-change of a subset of genes that were more greatly reduced by combination of Compound 5 with either nintedanib or pirfenidone (striped bars) than by individual treatments (solid bars). Changes that are significant (adj. p<0.05) are noted with an *.

### DETAILED DESCRIPTION OF THE INVENTION

The present disclosure provides, inter alia, compounds of formula (A), and variations thereof, or a salt thereof, pharmaceutical compositions comprising compounds of formula (A) or a salt thereof, and methods of using such compounds and compositions in treating fibrotic diseases.

The present disclosure provides, inter alia, compounds of formula (I), and variations thereof, or a salt thereof, pharmaceutical compositions comprising compounds of formula (I) or a salt thereof, and methods of using such compounds and compositions in treating fibrotic diseases.

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

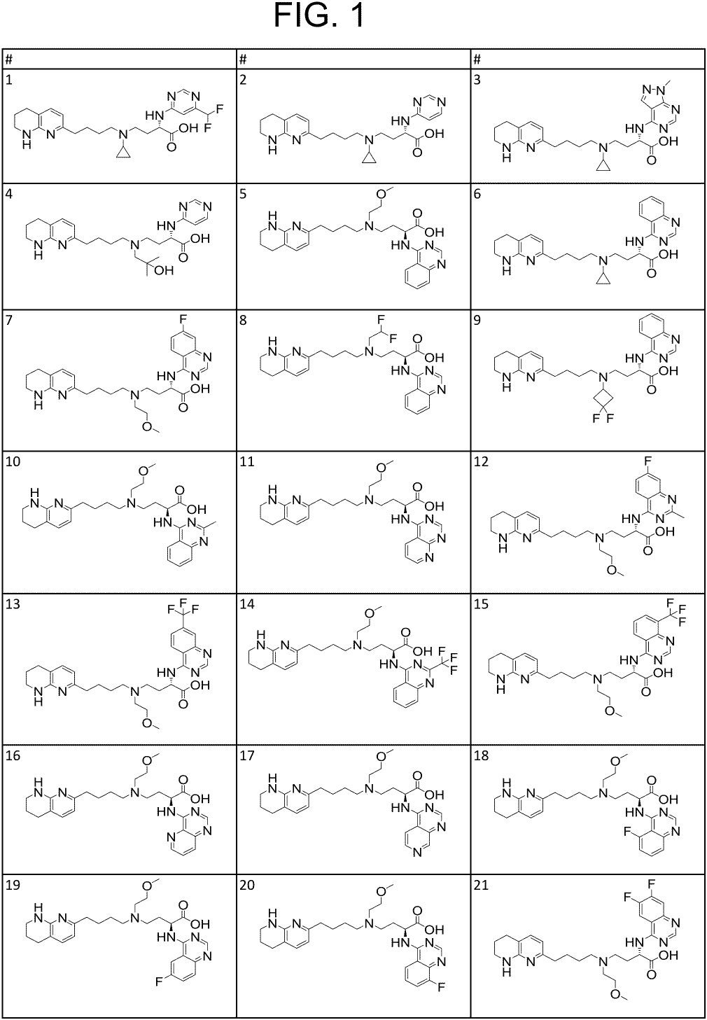

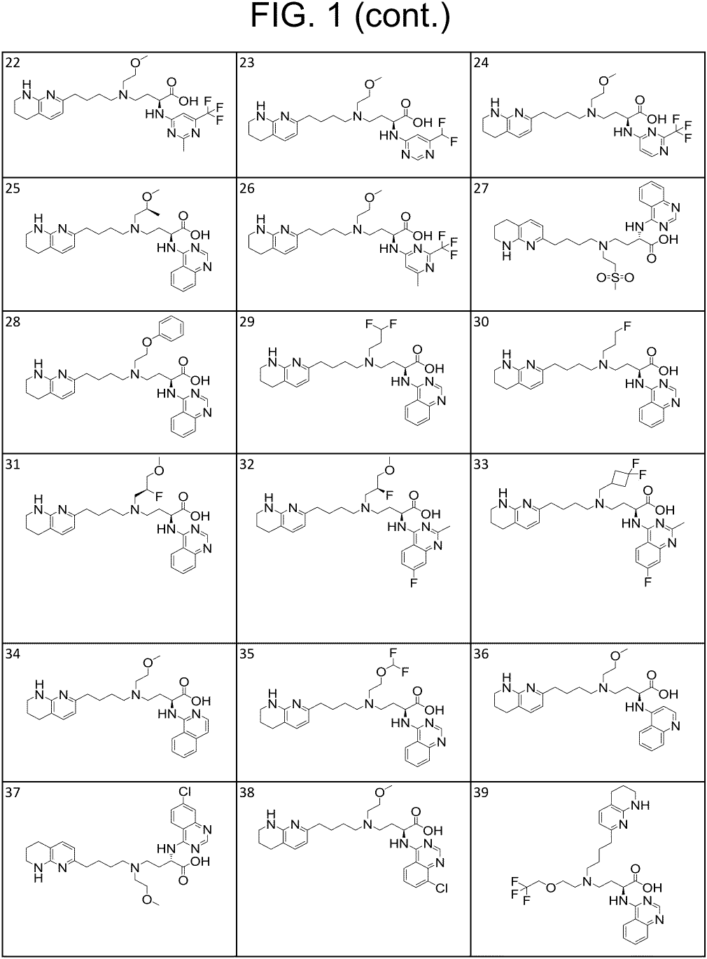

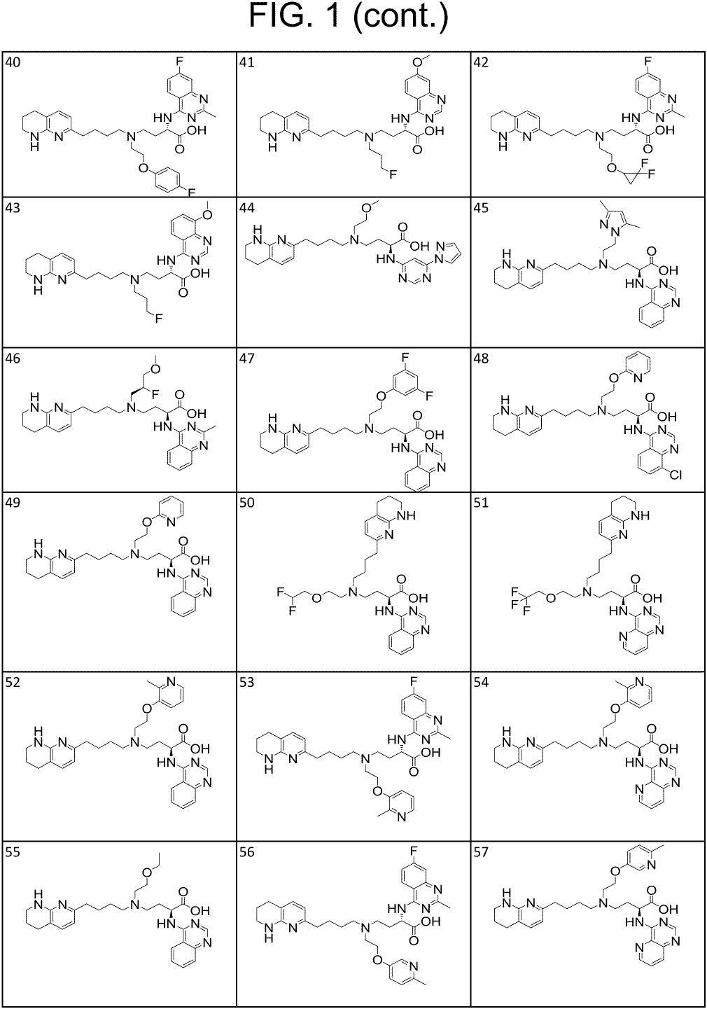

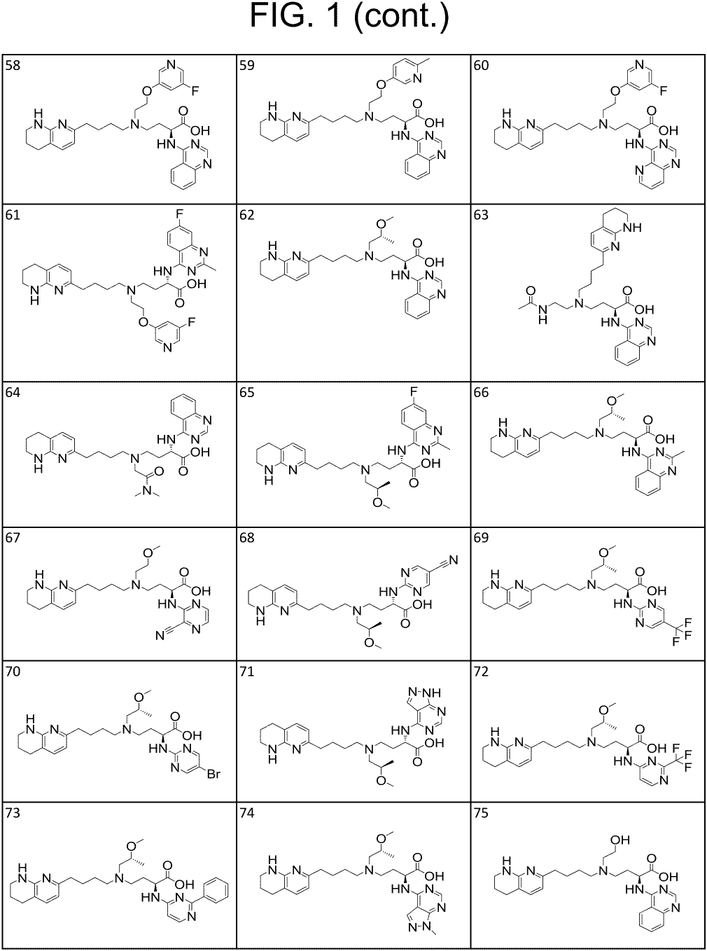

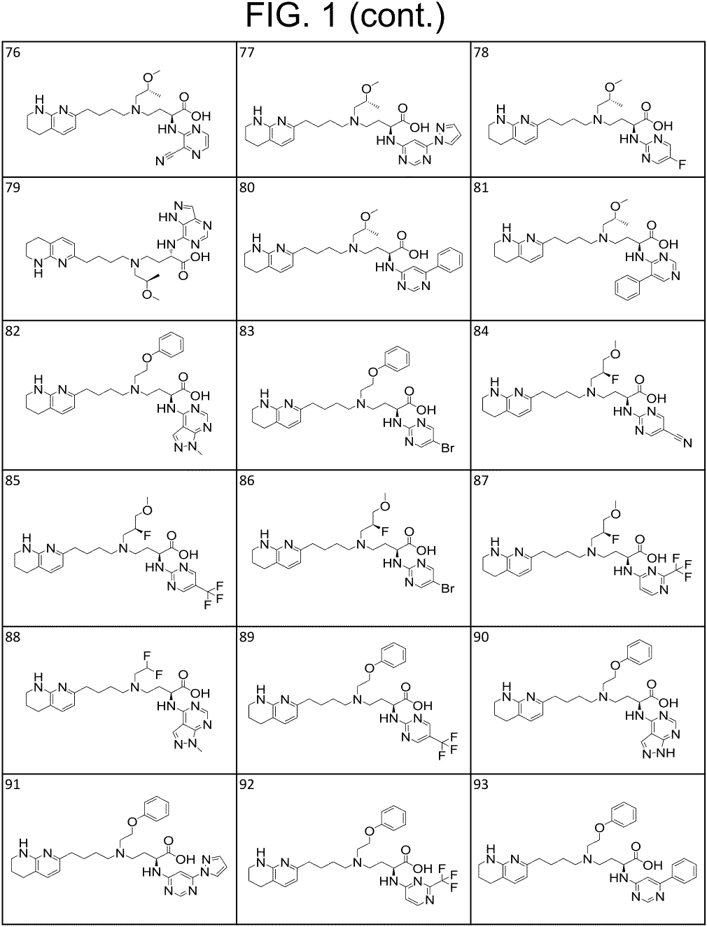

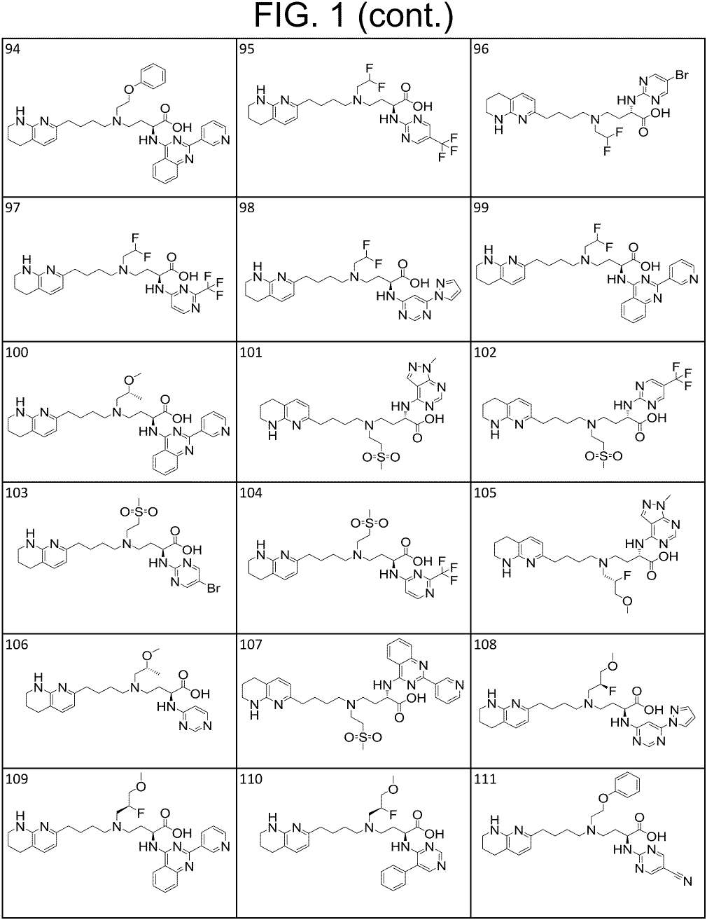

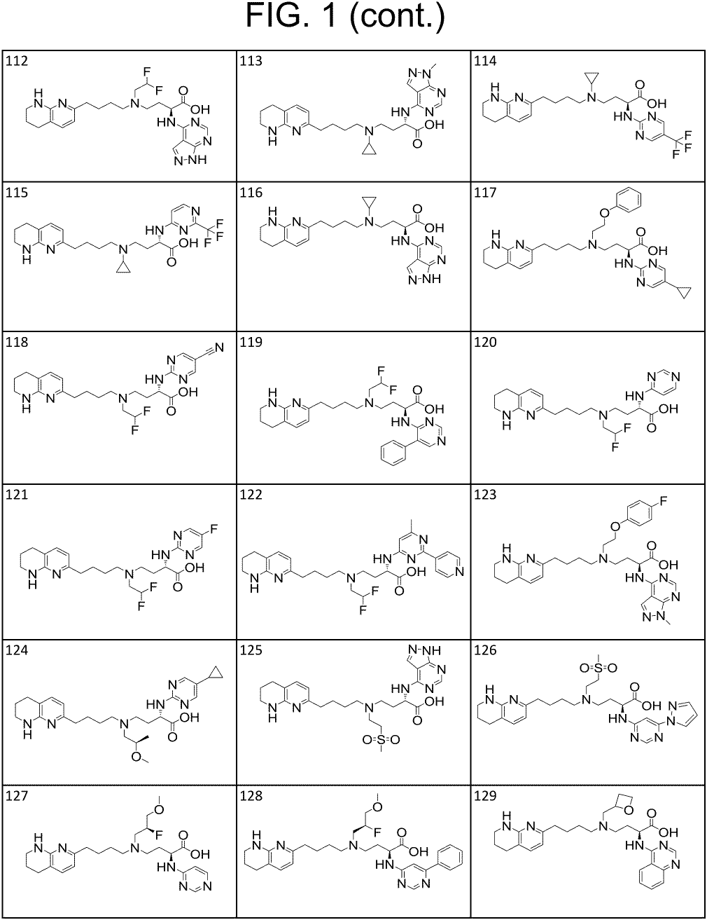

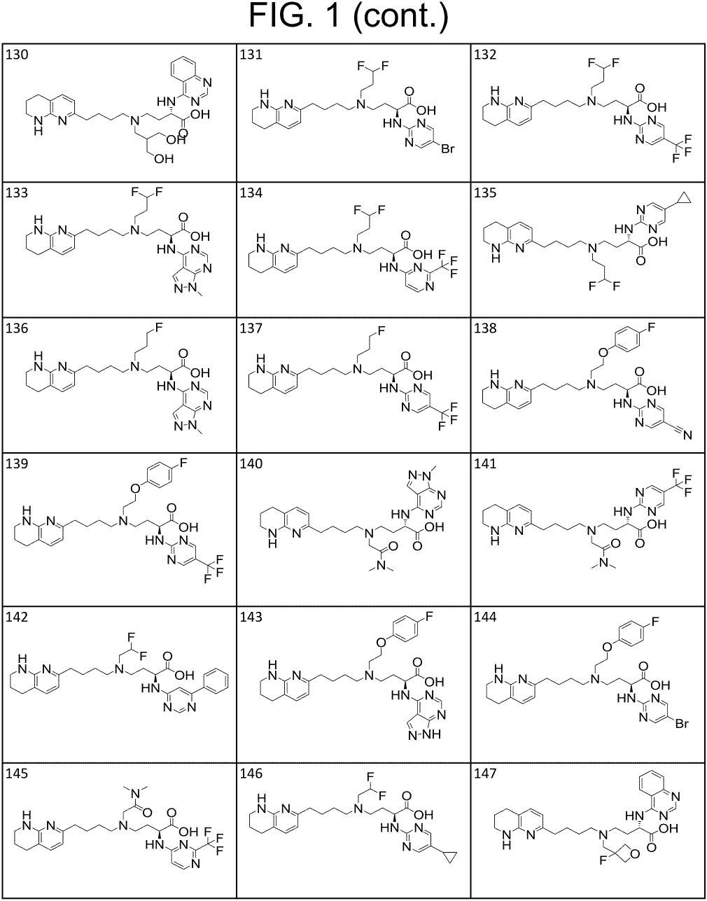

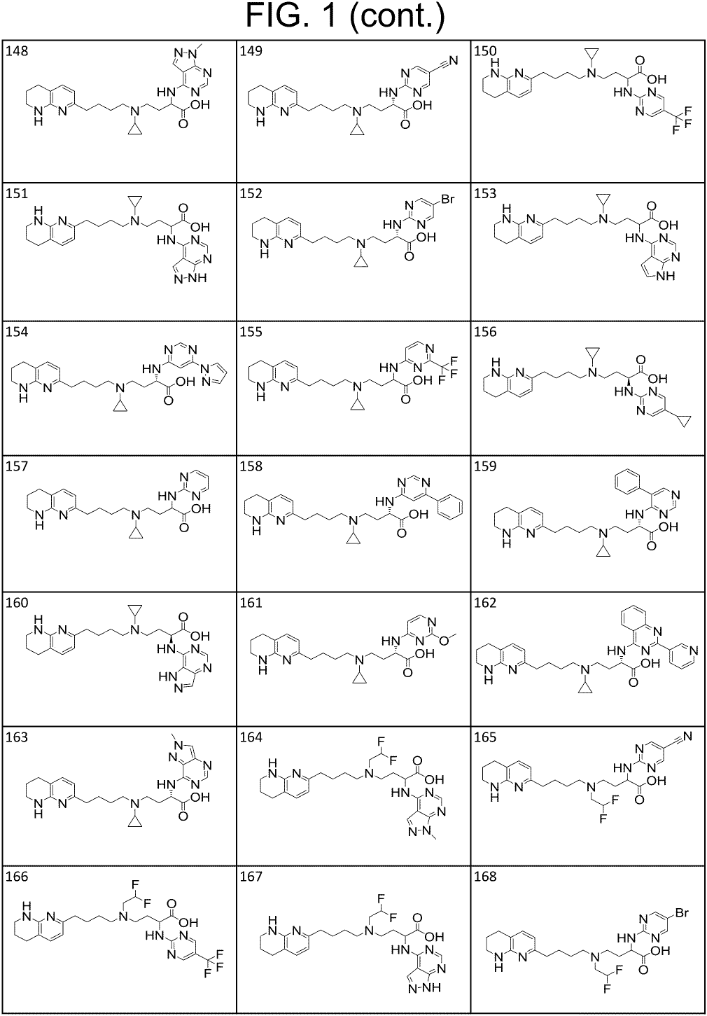

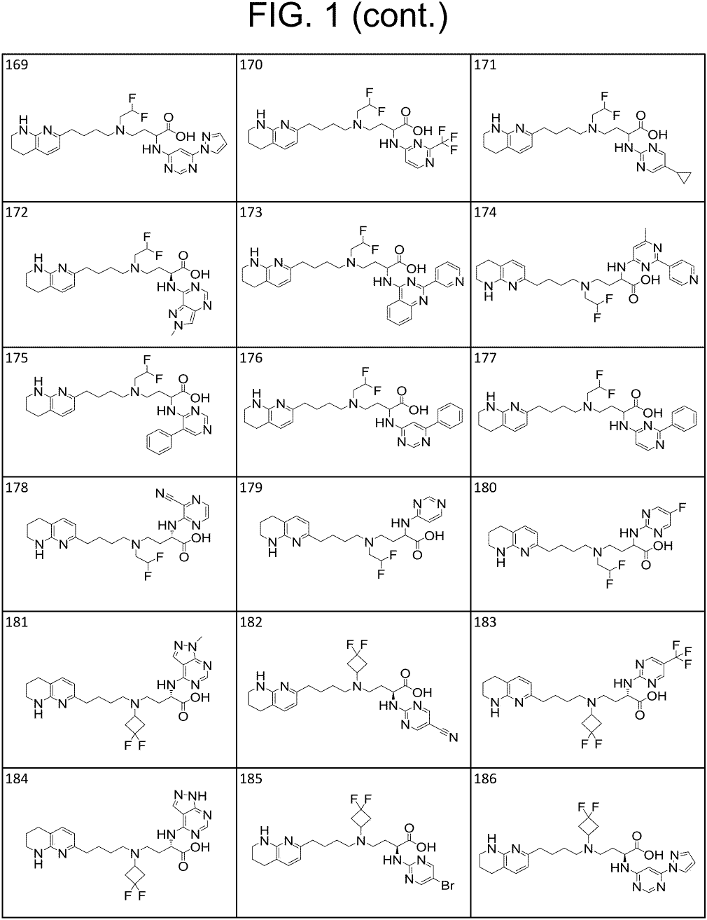

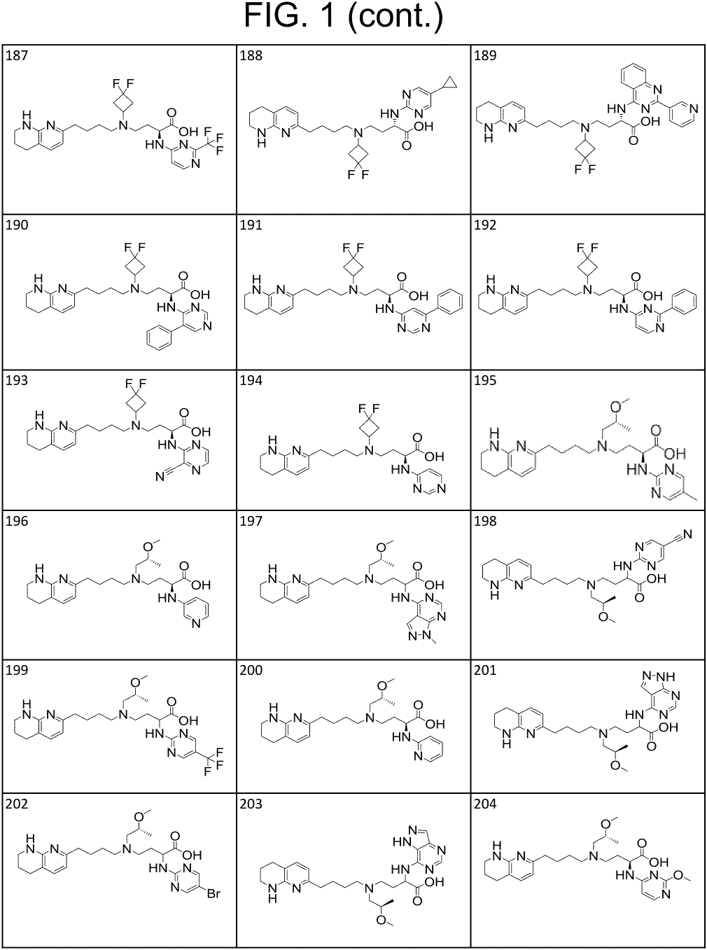

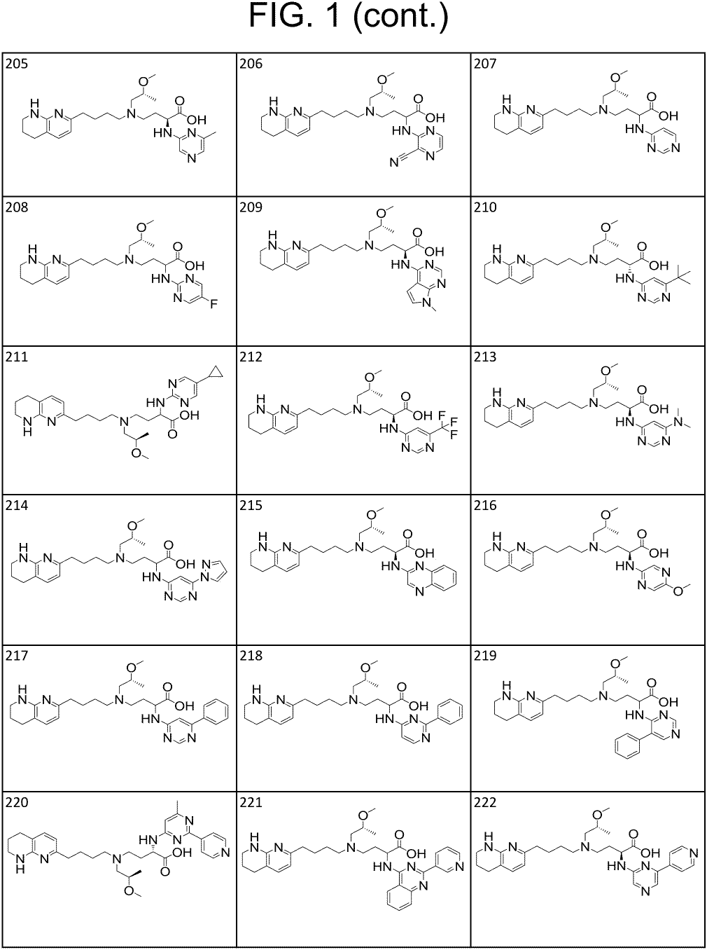

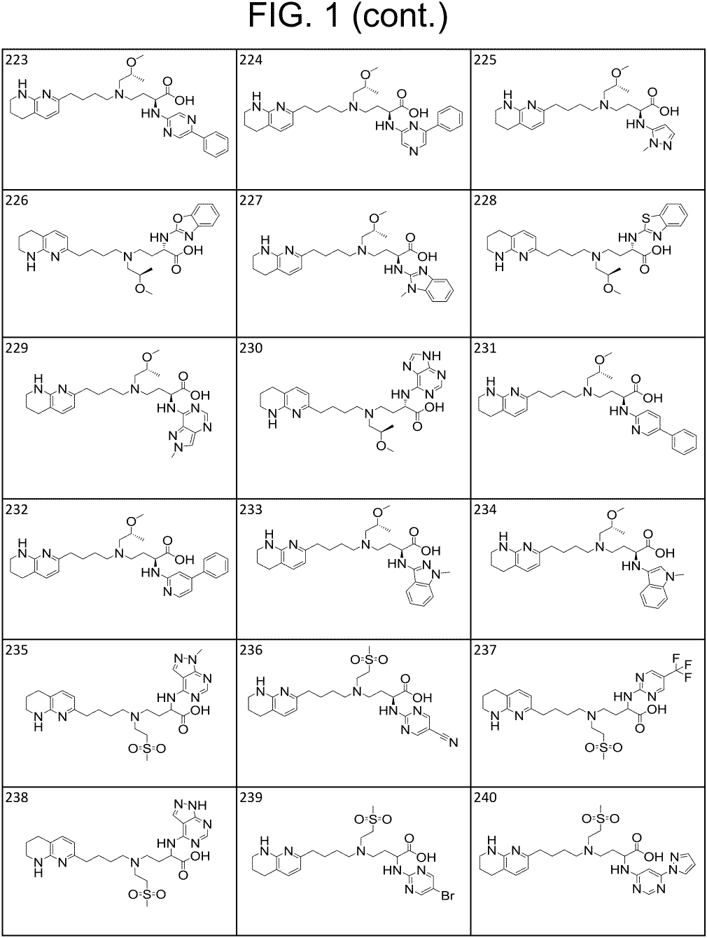

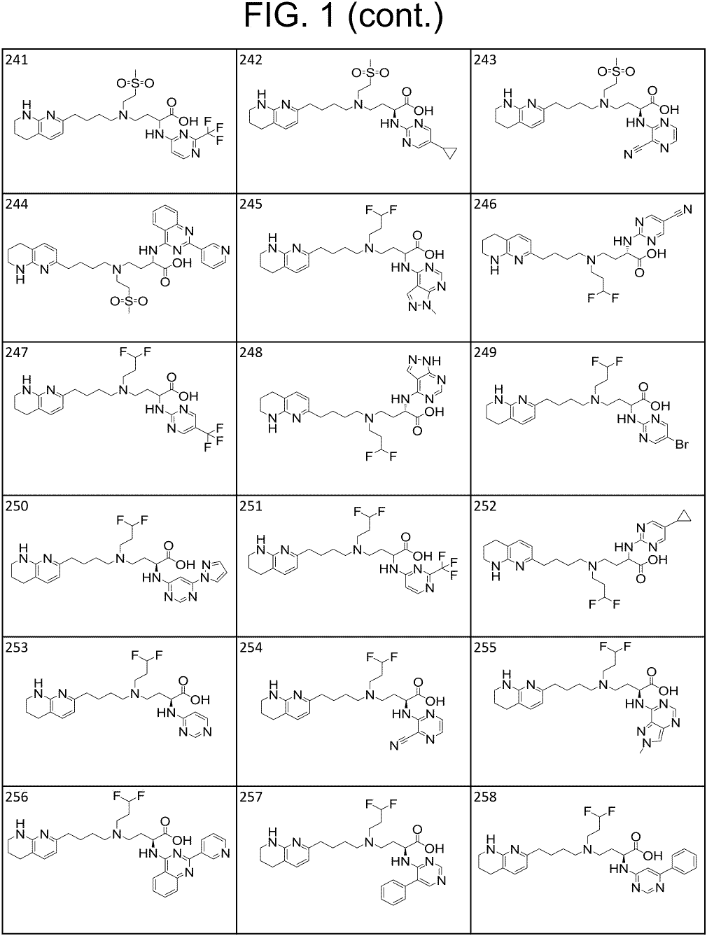

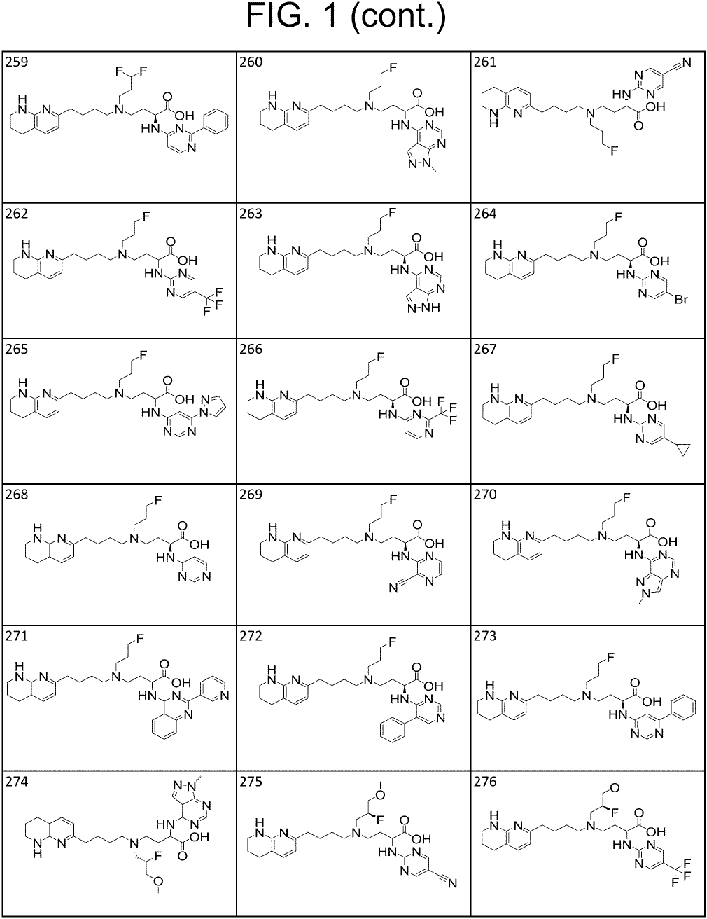

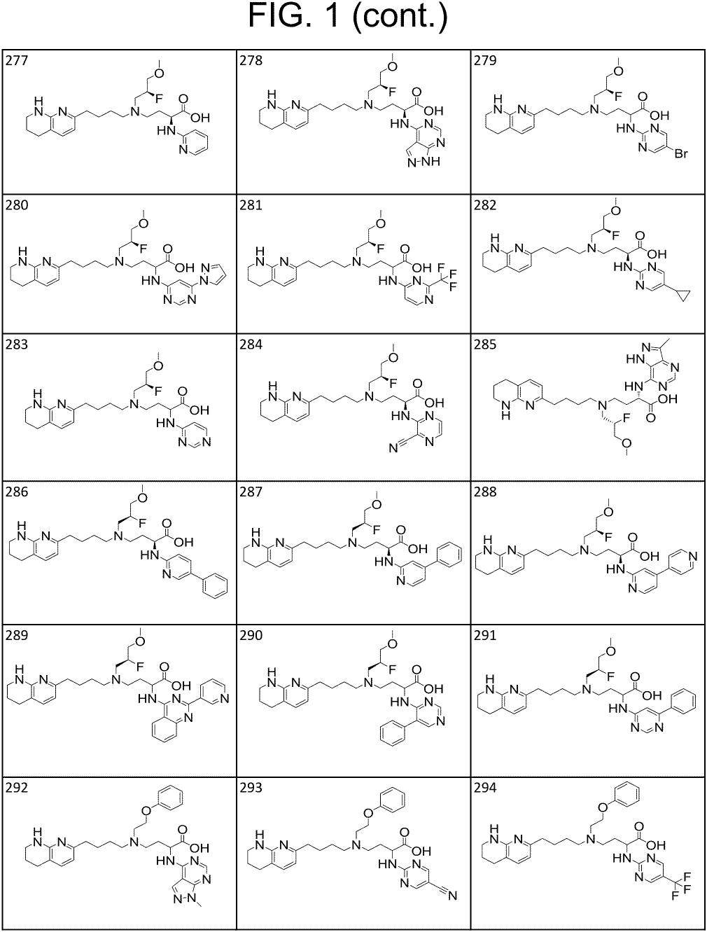

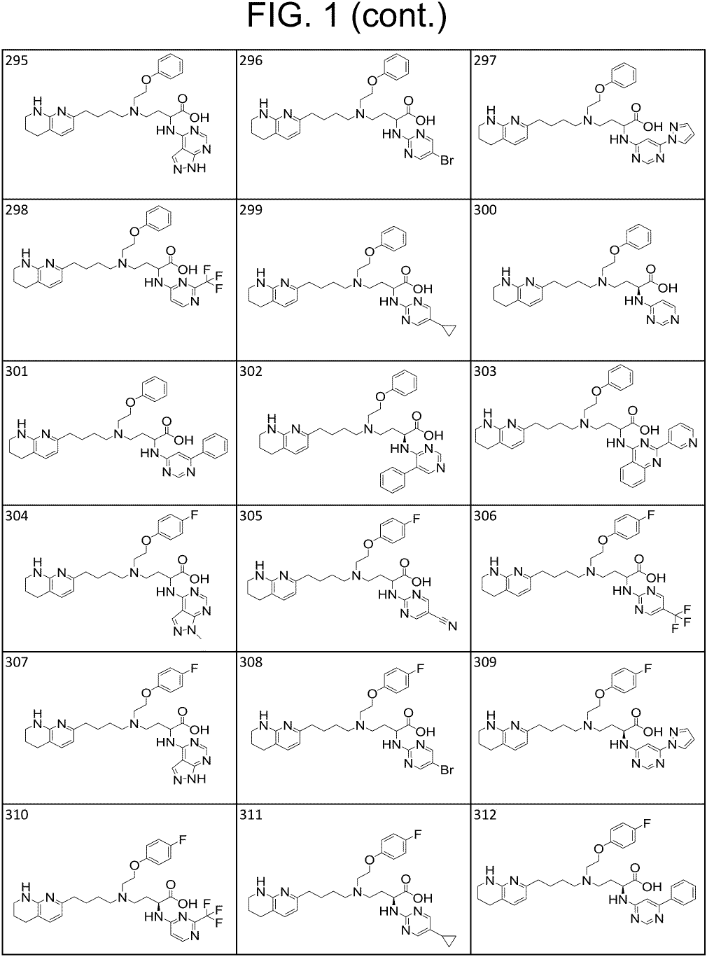

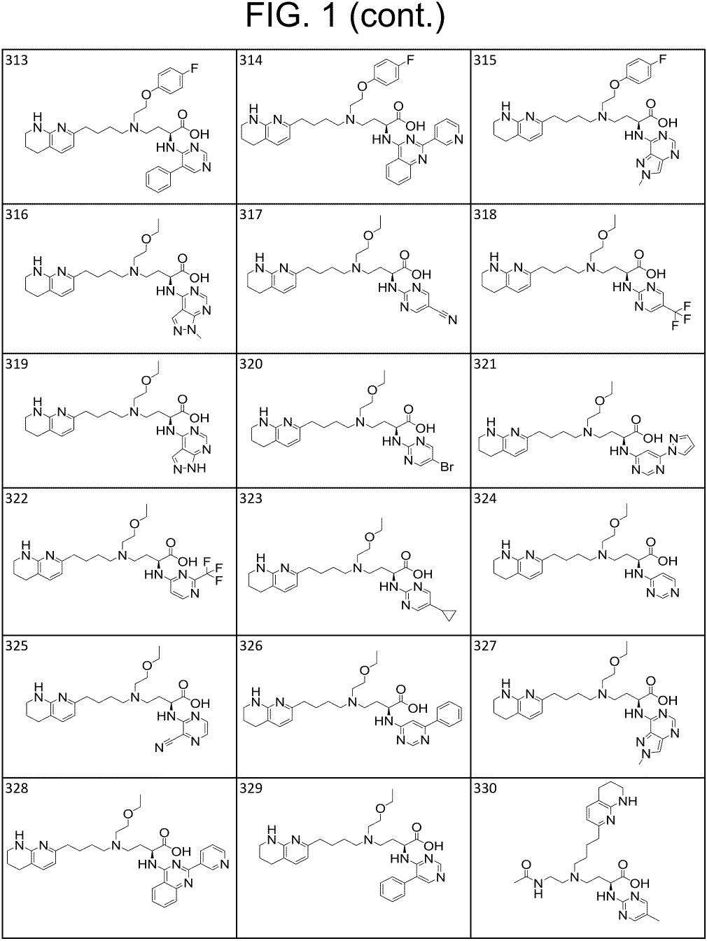

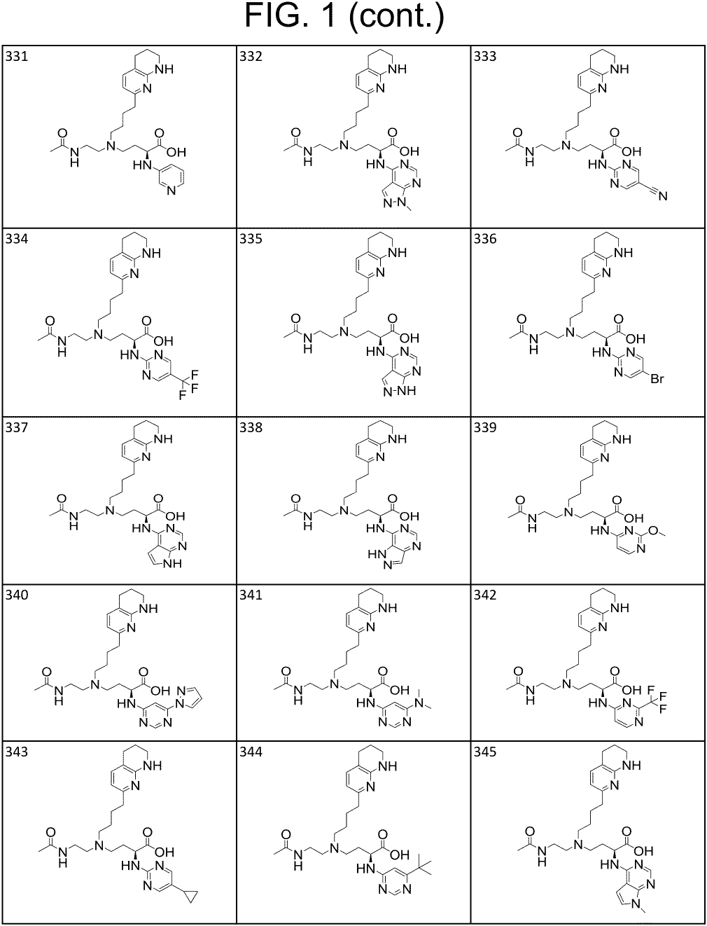

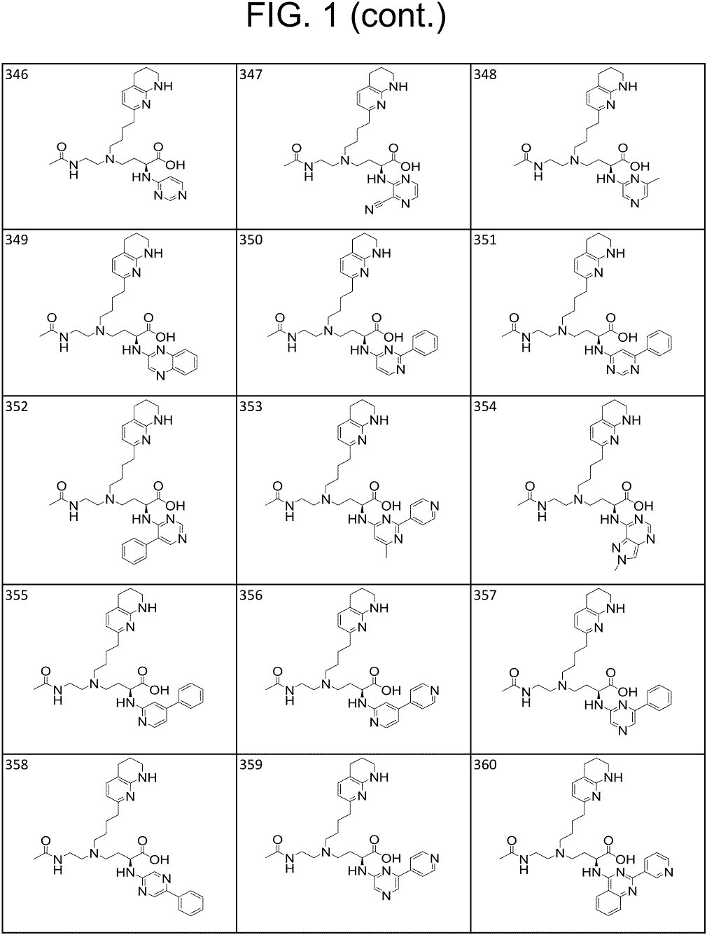

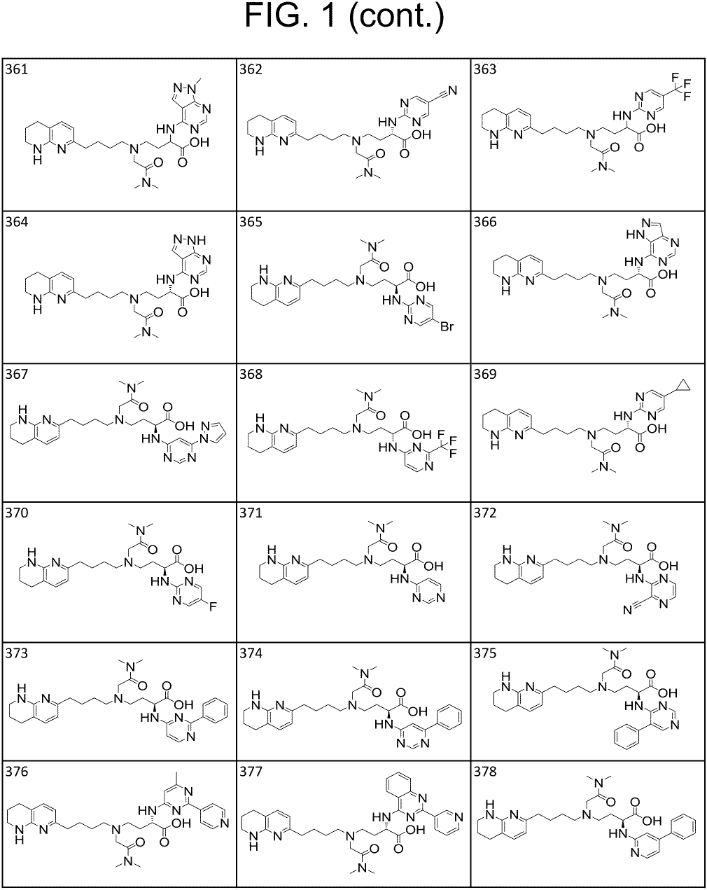

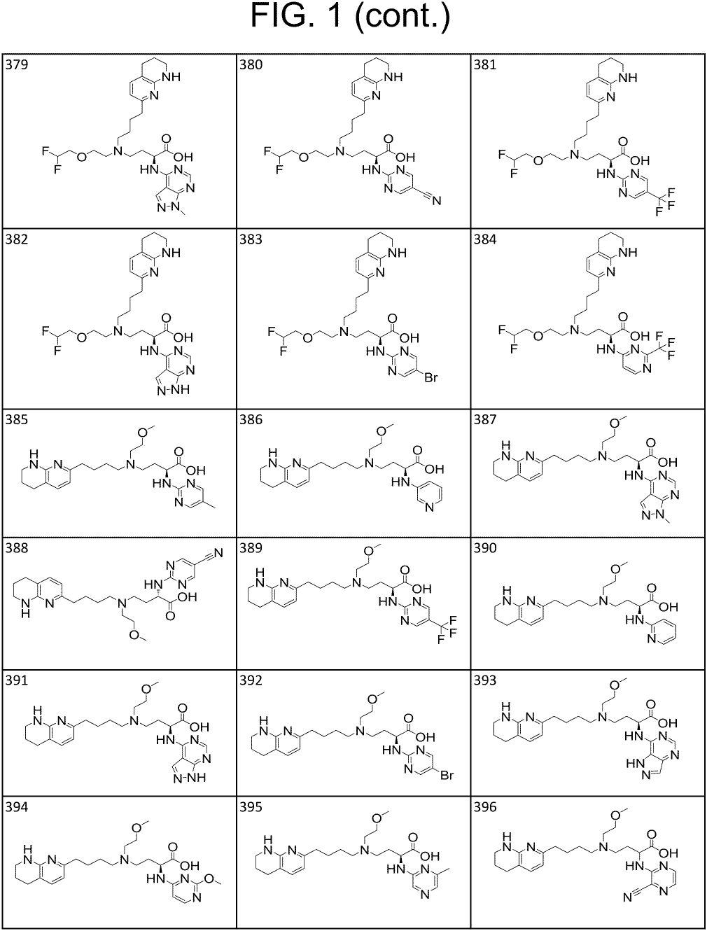

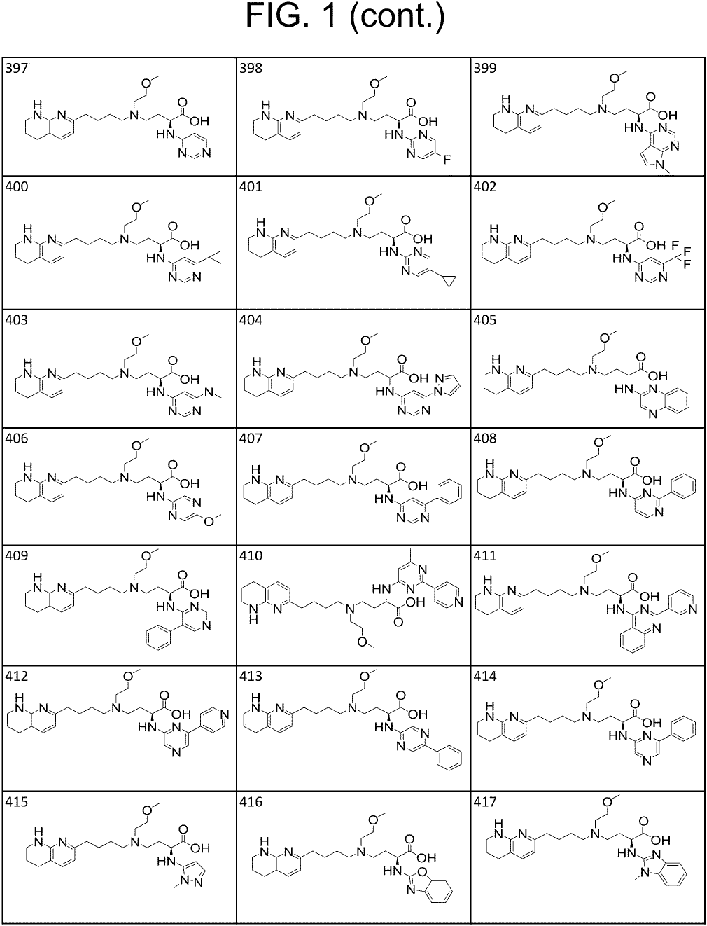

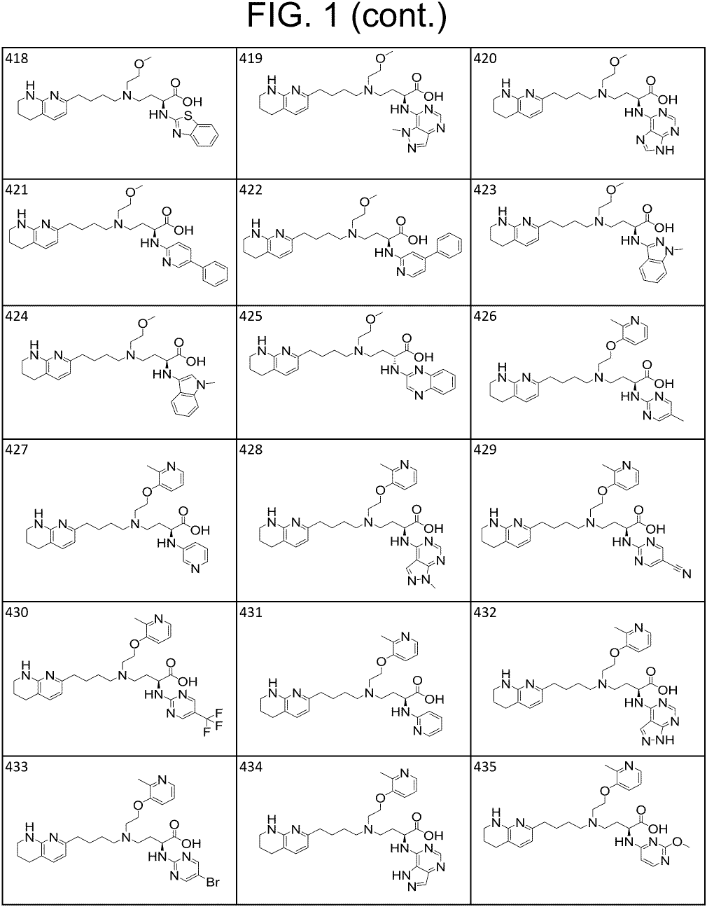

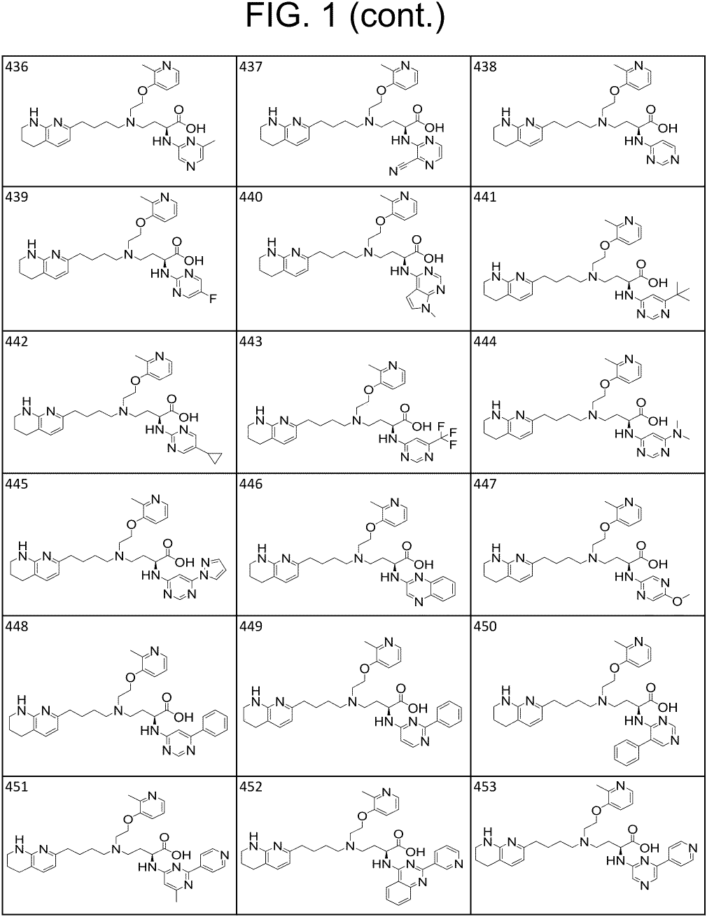

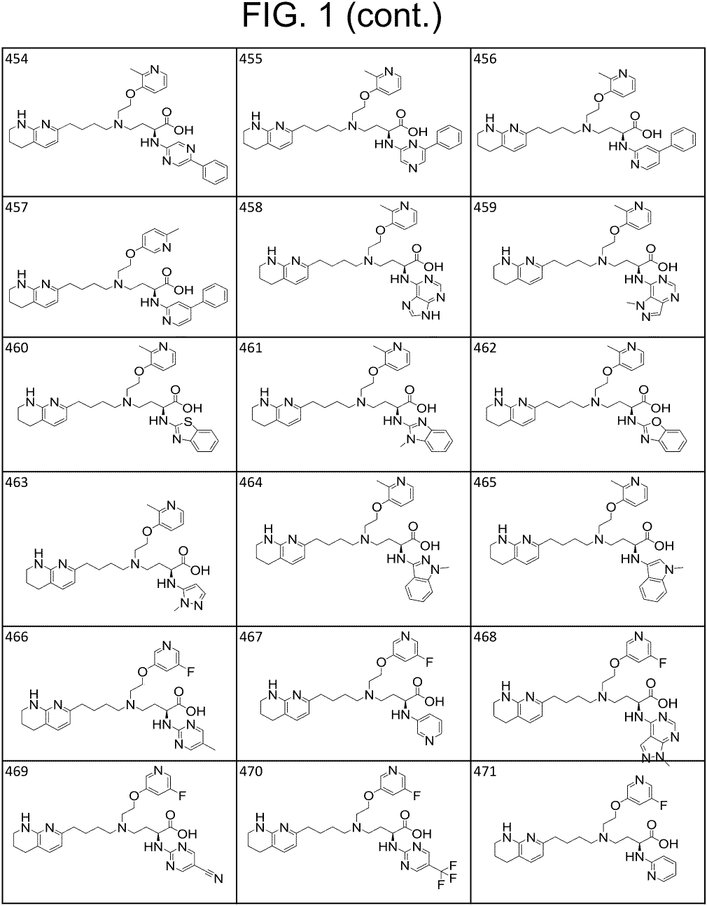

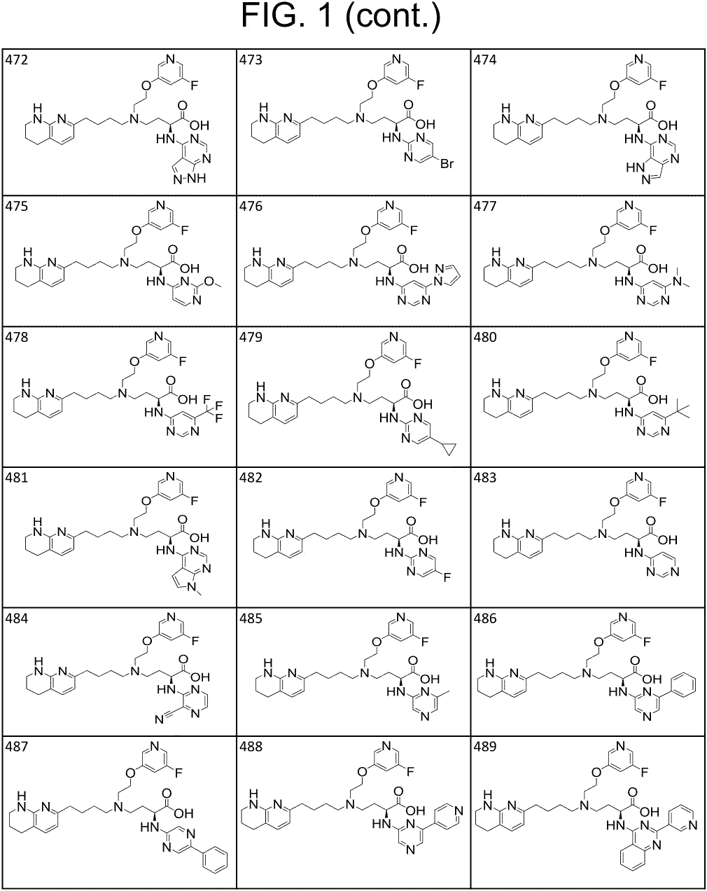

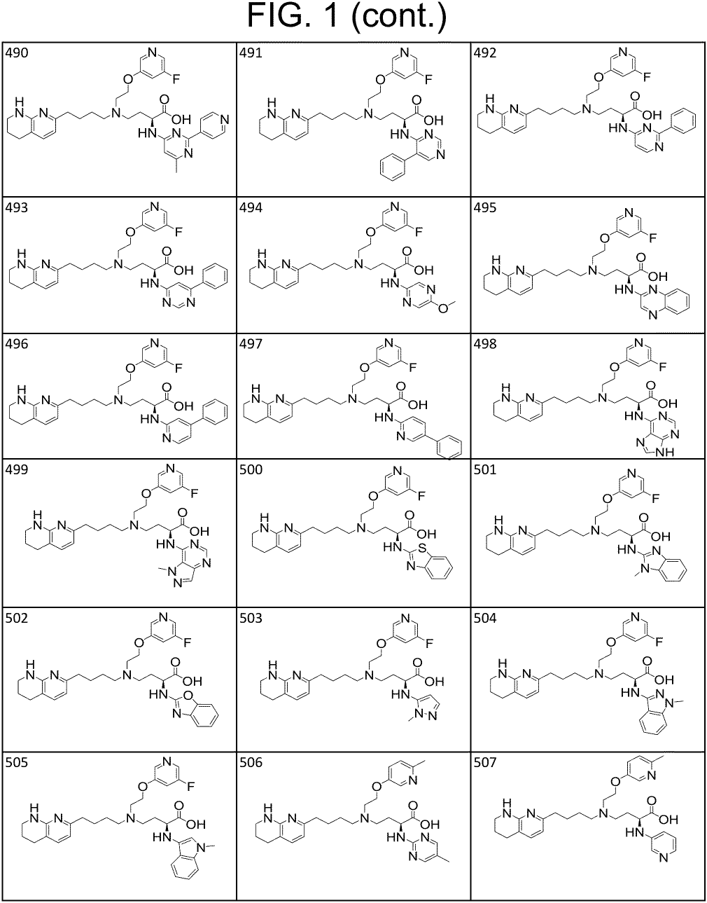

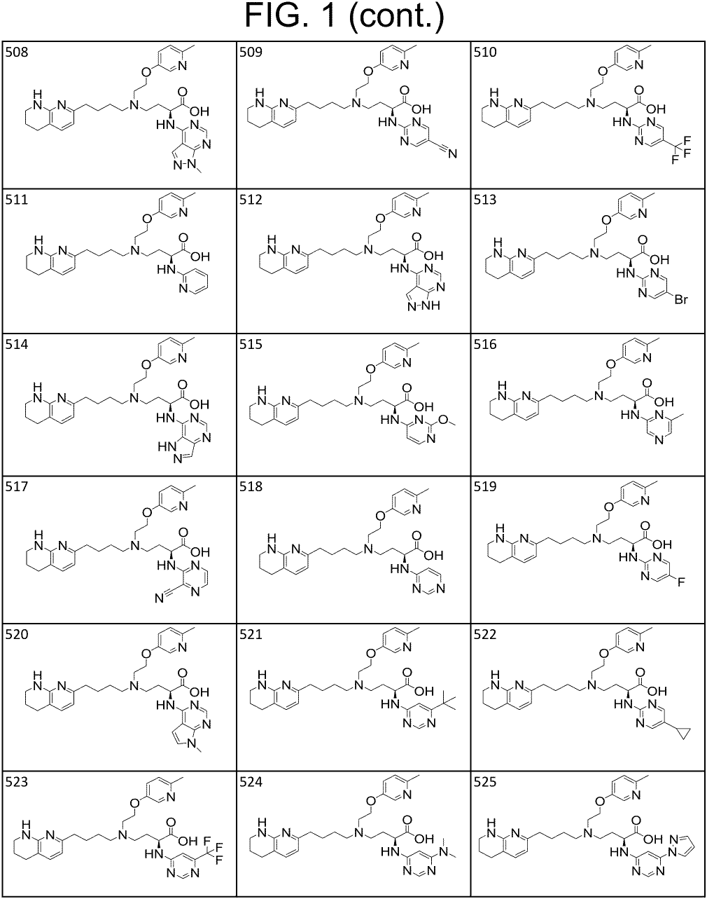

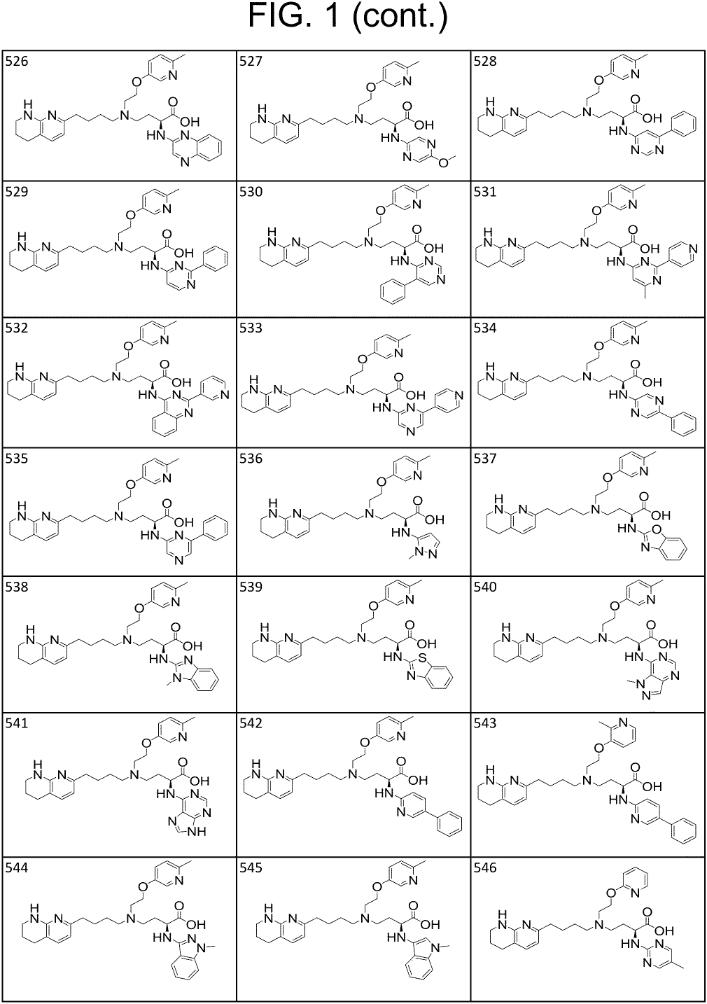

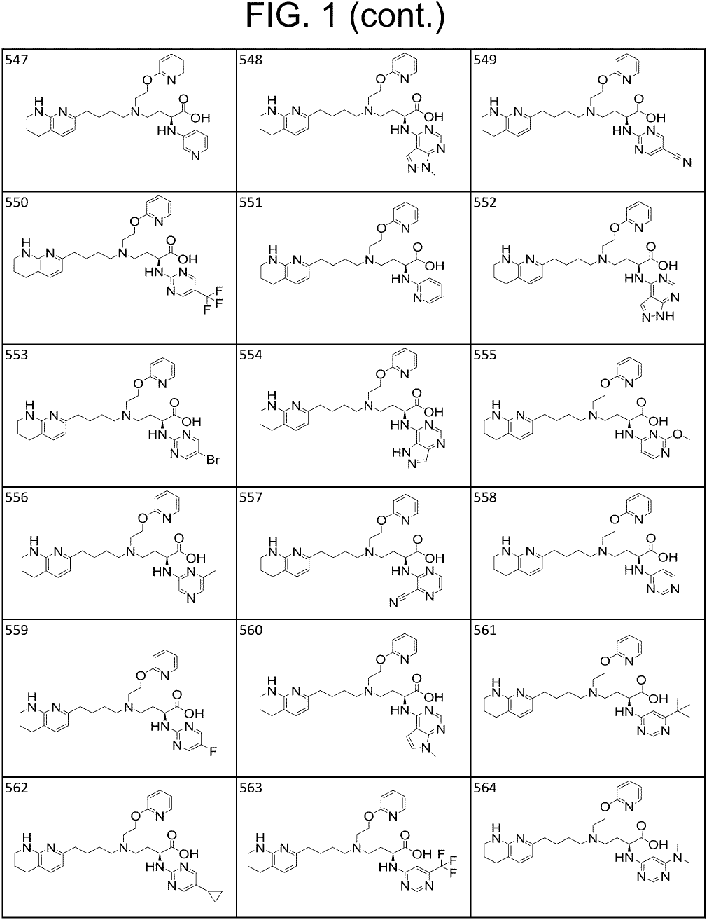

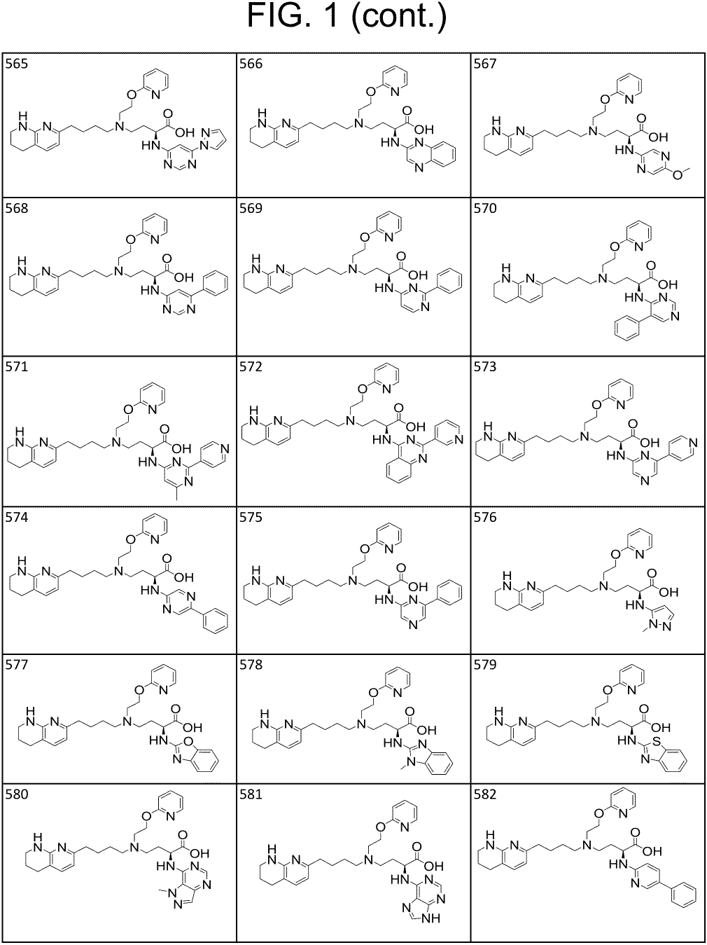

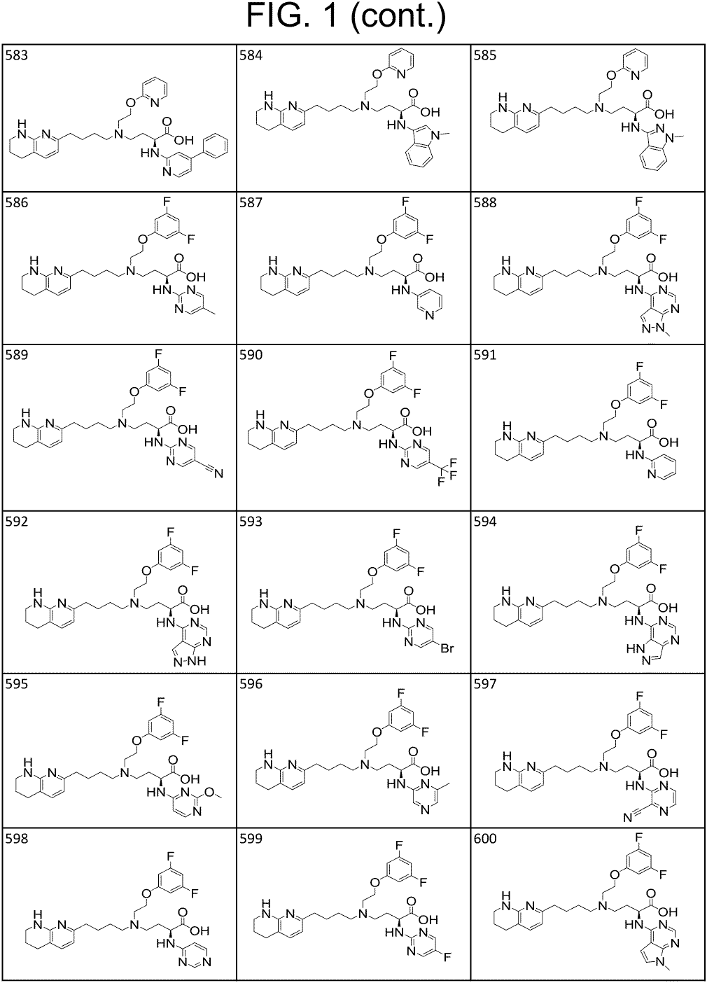

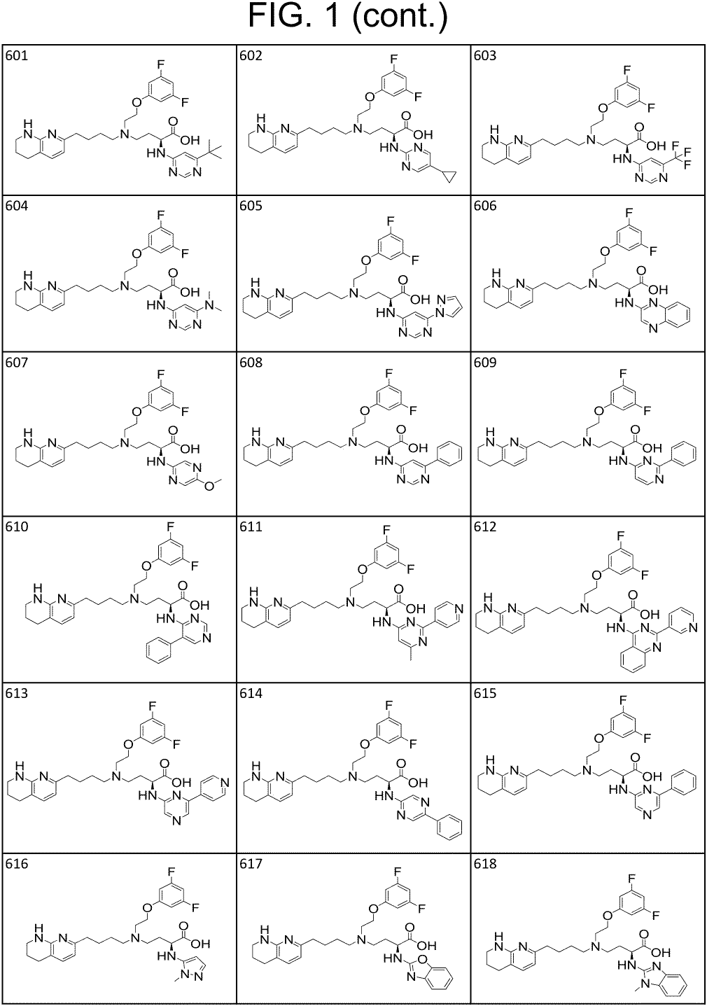

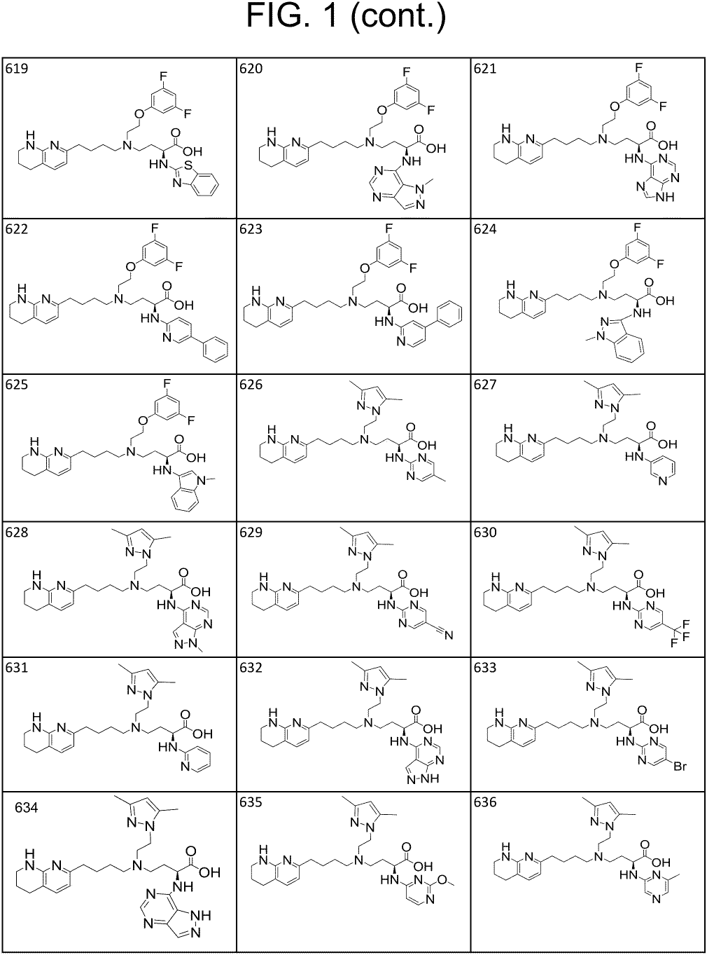

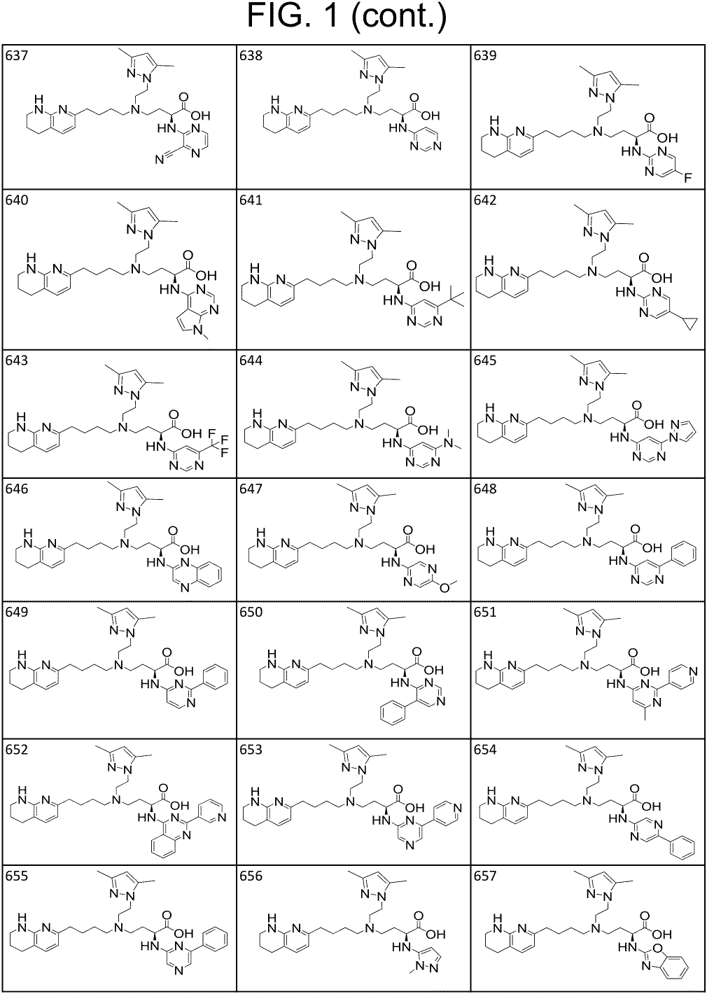

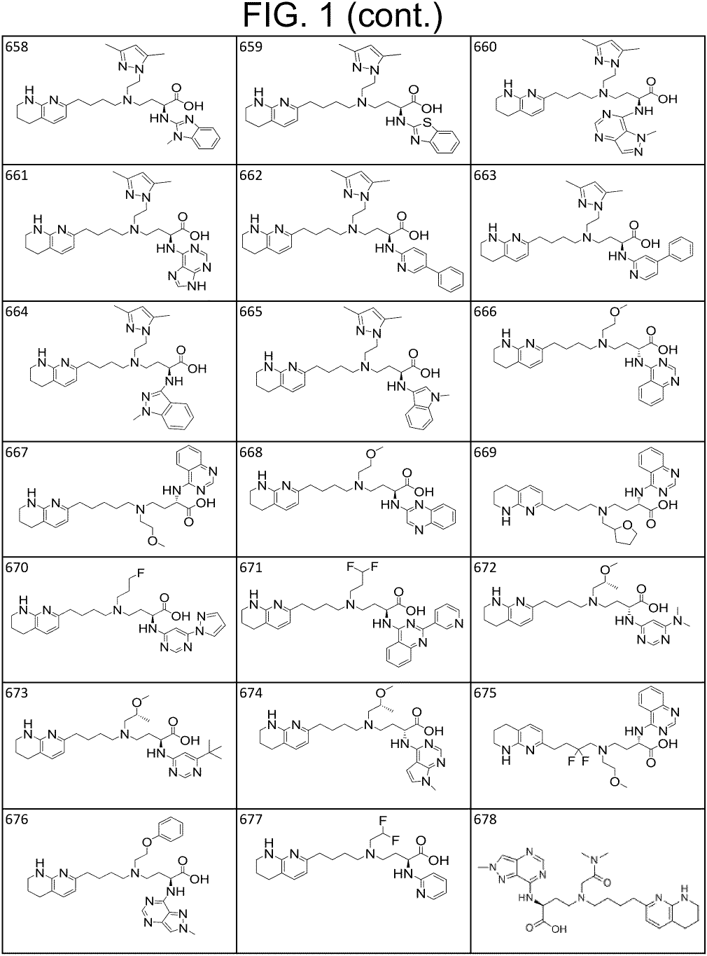

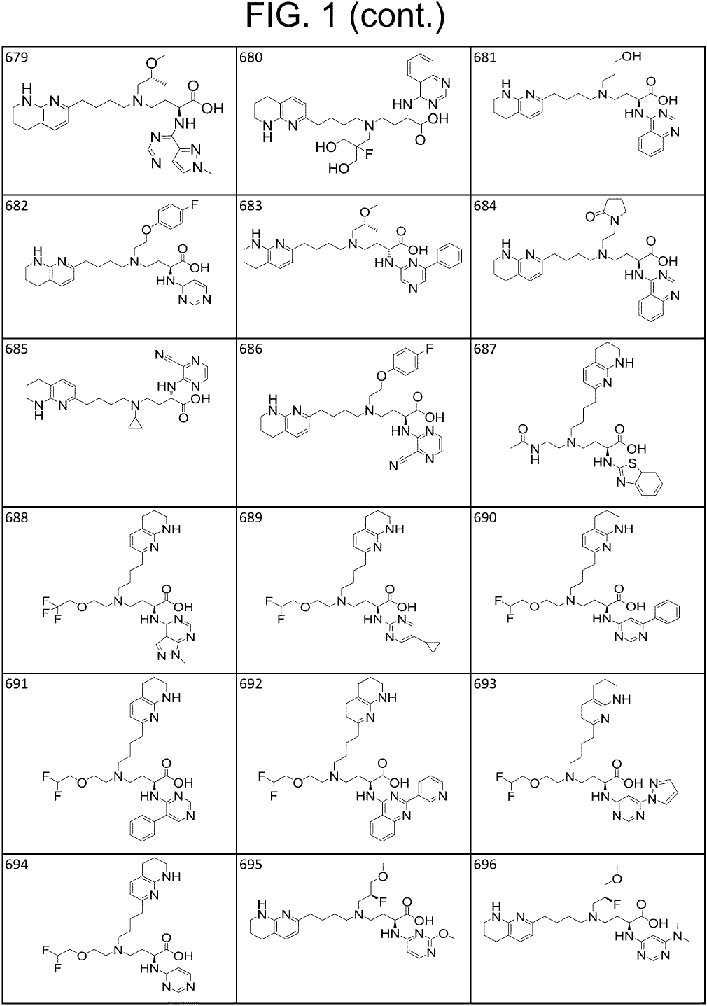

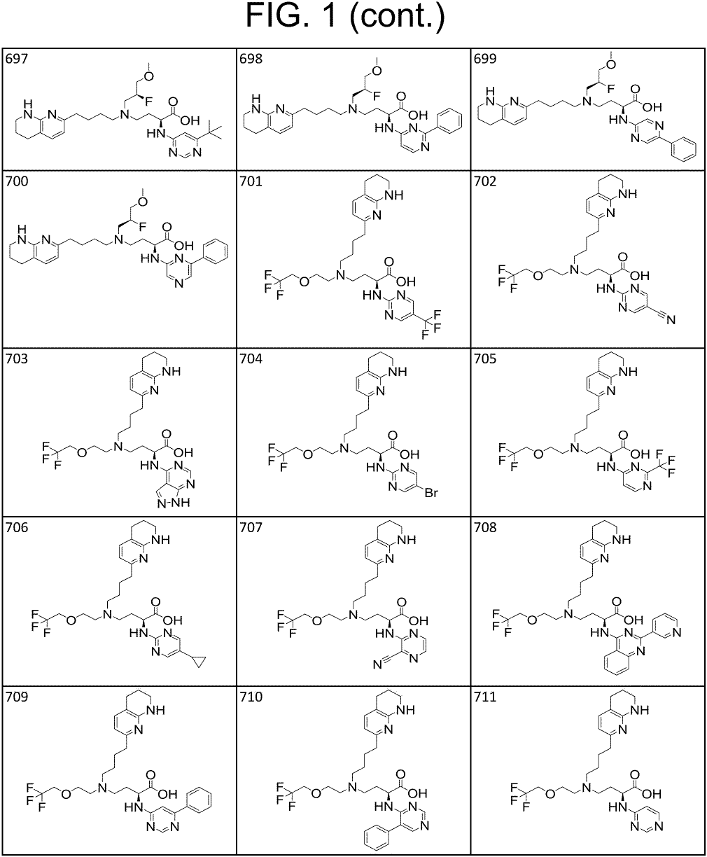

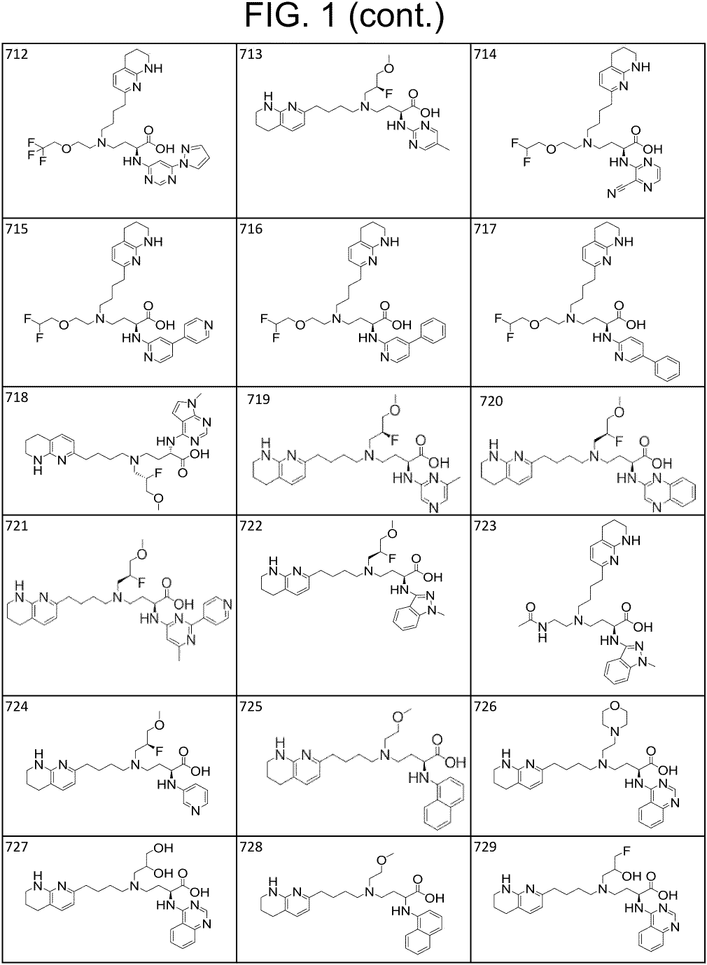

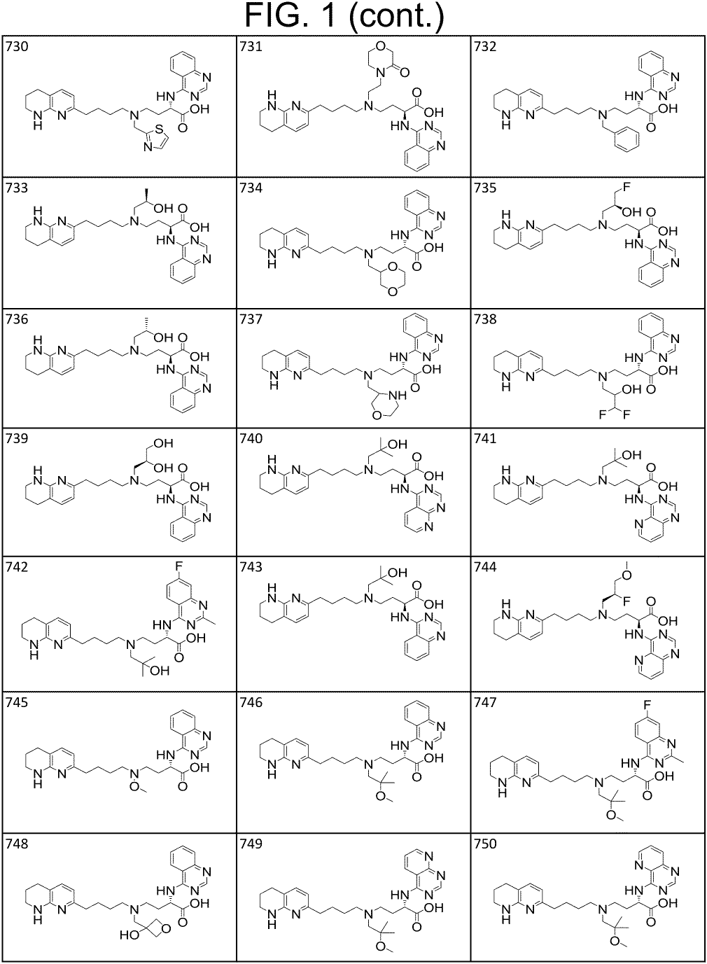

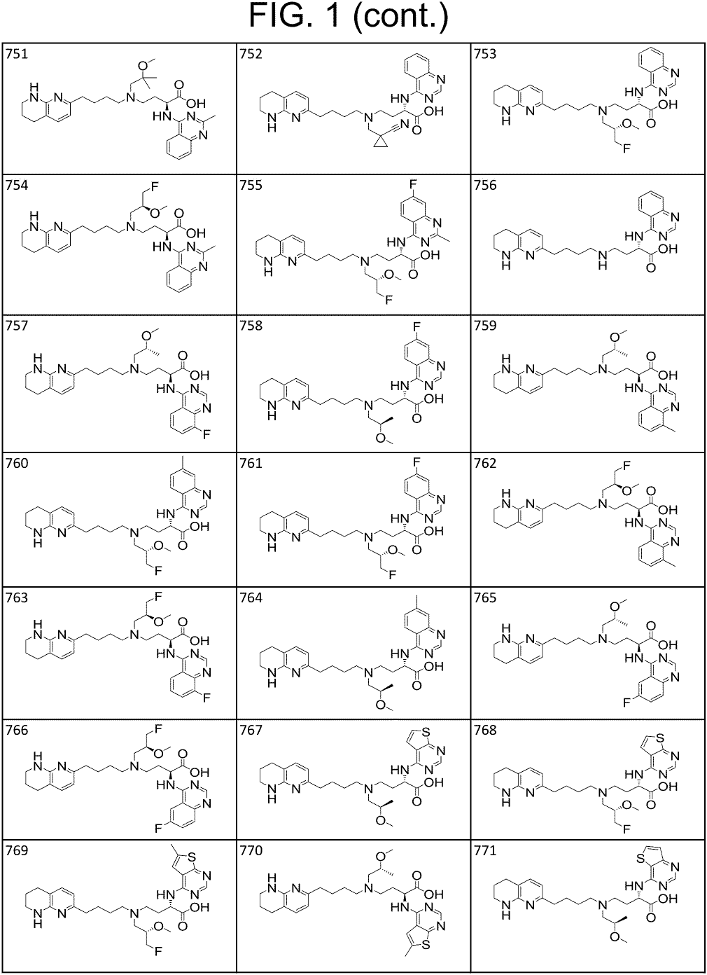

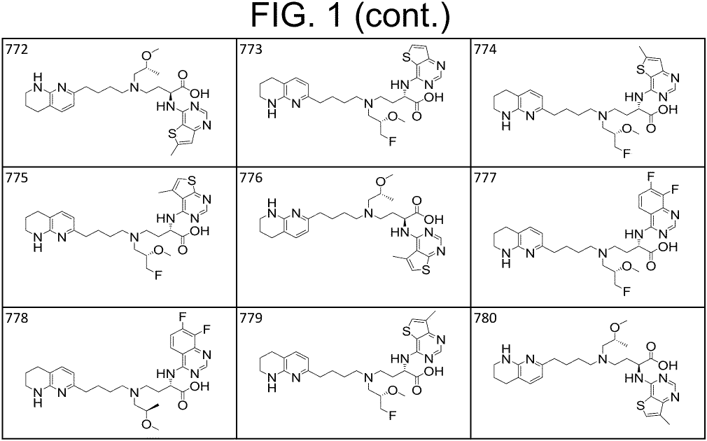

As used herein, a “small molecule” is an organic molecule characterized by a mass of less than 900 daltons. Non-limiting examples of small molecules include the compounds depicted in FIG. 1 or a salt thereof.

“Alkyl” as used herein refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Alkenyl” as used herein refers to and includes, unless otherwise stated, an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). An alkenyl group may have “cis” or “trans” configurations, or alternatively have “E” or “Z” configurations. Particular alkenyl groups are those having 2 to 20 carbon atoms (a “C₂-C₂₀ alkenyl”), having 6 to 10 carbon atoms (a “C₆-C₁₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, pent-1-enyl, pent-2-enyl, hex-1-enyl, hex-2-enyl, hex-3-enyl, and the like.

“Alkenylene” as used herein refers to the same residues as alkenyl, but having bivalency. Particular alkenylene groups are those having 2 to 20 carbon atoms (a “C₂-C₂₀ alkenylene”), having 2 to 10 carbon atoms (a “C₂-C₁₀ alkenylene”), having 6 to 10 carbon atoms (a “C₆-C₁₀ alkenylene”), having 2 to 6 carbon atoms (a “C₂-C₆ alkenylene”), 2 to 4 carbon atoms (a “C₂-C₄ alkenylene”) or 2 to 3 carbon atoms (a “C₂-C₃ alkenylene”). Examples of alkenylene include, but are not limited to, groups such as ethenylene (or vinylene) (—CH═CH—), propenylene (-CH=CHCH₂-), 1,4-but-1-enylene (—CH═CH—CH₂CH₂—), 1,4-but-2-enylene (-CH₂CH=CHCH₂-), 1,6-hex-1-enylene (—CH═CH—(CH₂)₃CH₂—), and the like.

“Alkynyl” as used herein refers to and includes, unless otherwise stated, an unsaturated linear (i.e., unbranched) or branched univalent hydrocarbon chain or combination thereof, having at least one site of acetylenic unsaturation (i.e., having at least one moiety of the formula C═C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 6 to 10 carbon atoms (a “C₆-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, and the like.

“Alkynylene” as used herein refers to the same residues as alkynyl, but having bivalency. Particular alkynylene groups are those having 2 to 20 carbon atoms (a “C₂-C₂₀ alkynylene”), having 2 to 10 carbon atoms (a “C₂-C₁₀ alkynylene”), having 6 to 10 carbon atoms (a “C₆-C₁₀ alkynylene”), having 2 to 6 carbon atoms (a “C₂-C₆ alkynylene”), 2 to 4 carbon atoms (a “C₂-C₄ alkynylene”) or 2 to 3 carbon atoms (a “C₂-C₃ alkynylene”). Examples of alkynylene include, but are not limited to, groups such as ethynylene (or acetylenylene) (—C═C—), propynylene (—C═CCH₂—), and the like.

“Cycloalkyl” as used herein refers to and includes, unless otherwise stated, saturated cyclic univalent hydrocarbon structures, having the number of carbon atoms designated (i.e., C₃-C₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃-C₈ cycloalkyl”), having 3 to 6 annular carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like.

“Cycloalkylene” as used herein refers to the same residues as cycloalkyl, but having bivalency. Cycloalkylene can consist of one ring or multiple rings which may be fused, spiro or bridged, or combinations thereof. Particular cycloalkylene groups are those having from 3 to 12 annular carbon atoms. A preferred cycloalkylene is a cyclic hydrocarbon having from 3 to 8 annular carbon atoms (a “C₃-C₈ cycloalkylene”), having 3 to 6 carbon atoms (a “C₃-C₆ cycloalkylene”), or having from 3 to 4 annular carbon atoms (a “C₃-C₄ cycloalkylene”). Examples of cycloalkylene include, but are not limited to, cyclopropylene, cyclobutylene, cyclopentylene, cyclohexylene, cycloheptylene, norbomylene, and the like. A cycloalkylene may attach to the remaining structures via the same ring carbon atom or different ring carbon atoms. When a cycloalkylene attaches to the remaining structures via two different ring carbon atoms, the connecting bonds may be cis- or trans- to each other. For example, cyclopropylene may include 1,1-cyclopropylene and 1,2-cyclopropylene (e.g., cis-1,2-cyclopropylene or trans-1,2-cyclopropylene), or a mixture thereof.

“Cycloalkenyl” refers to and includes, unless otherwise stated, an unsaturated cyclic non-aromatic univalent hydrocarbon structure, having at least one site of olefinic unsaturation (i.e., having at least one moiety of the formula C═C) and having the number of carbon atoms designated (i.e., C₃-C₁₀ means three to ten carbon atoms). Cycloalkenyl can consist of one ring, such as cyclohexenyl, or multiple rings, such as norbomenyl. A preferred cycloalkenyl is an unsaturated cyclic hydrocarbon having from 3 to 8 annular carbon atoms (a “C₃-C₈ cycloalkenyl”). Examples of cycloalkenyl groups include, but are not limited to, cyclopropenyl, cyclobutenyl, cyclopentenyl, cyclohexenyl, norbomenyl, and the like.

“Cycloalkenylene” as used herein refers to the same residues as cycloalkenyl, but having bivalency.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Arylene” as used herein refers to the same residues as aryl, but having bivalency. Particular arylene groups are those having from 6 to 14 annular carbon atoms (a “C₆-C₁₄ arylene”).

“Heteroaryl” as used herein refers to an unsaturated aromatic cyclic group having from 1 to 14 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen and sulfur, 5 to 10-membered rings having 1 to 8 annular carbon atoms and 1 to 4 annular heteroatoms independently selected from nitrogen, oxygen and sulfur, or 5, 6 or 7-membered rings having 1 to 5 annular carbon atoms and 1 to 4 annular heteroatoms independently selected from nitrogen, oxygen and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen and sulfur. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position. A heteroaryl group may be connected to the parent structure at a ring carbon atom or a ring heteroatom.

“Heteroarylene” as used herein refers to the same residues as heteroaryl, but having bivalency.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof, but excludes heteroaryl groups. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur.

“Heterocyclylene” as used herein refers to the same residues as heterocyclyl, but having bivalency.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (-OCF₃).

“Carbonyl” refers to the group C═O.

“Thiocarbonyl” refers to the group C═S.

“Oxo” refers to the moiety ═O.

“D” refers to deuterium (²H).

“T” refers to tritium (³H).

An alkyl group in which each hydrogen is replaced with deuterium is referred to as “perdeuterated.” An alkyl group in which each hydrogen is replaced with tritium is referred to as “pertritiated.”

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

It is understood that an optionally substituted moiety can be substituted with more than five substituents, if permitted by the number of valences available for substitution on the moiety. For example, a propyl group can be substituted with seven halogen atoms to provide a perhalopropyl group. The substituents may be the same or different.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. Also encompassed by “treatment” is a reduction of pathological consequence of fibrosis. The methods of the invention contemplate any one or more of these aspects of treatment.

As used herein, the term “effective amount” intends such amount of a compound of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc = “directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

Unless otherwise stated, “substantially pure” intends a composition that contains no more than 10% impurity, such as a composition comprising less than 9%, 7%, 5%, 3%, 1%, 0.5% impurity.

It is understood that aspects and embodiments described herein as “comprising” include “consisting of” and “consisting essentially of” embodiments.

### Compounds

In one aspect, provided is a compound of formula (A):

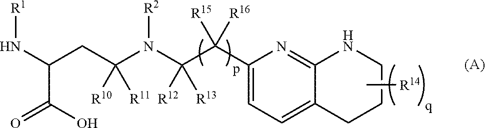

or a salt thereof, wherein:
R¹ is C₆-C₁₄ aryl or 5- to 10-membered heteroaryl wherein the C₆-C₁₄ aryl and 5- to 10-membered heteroaryl are optionally substituted by R^{1a};
R² is hydrogen; deuterium; C₁-C₆ alkyl optionally substituted by R^{2a}; —OH; —O—C₁—C₆ alkyl optionally substituted by R^{2a}; C₃-C₆ cycloalkyl optionally substituted by R^{2b}; —O—C₃—C₆ cycloalkyl optionally substituted by R^{2b}; 3- to 12-membered heterocyclyl optionally substituted by R^{2c}; or -S(O)₂R^{2d}; with the proviso that any carbon atom bonded directly to a nitrogen atom is optionally substituted with an R^{2a} moiety other than halogen;
each R^{1a} is independently C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, C₆-C₁₄ aryl, deuterium, halogen, —CN, -OR³, -SR³, -NR⁴R⁵, -NO₂, -C=NH(OR³), -C(O)R³, -OC(O)R³, -C(O)OR³, -C(O)NR⁴R⁵, -NR³C(O)R⁴, -NR³C(O)OR⁴, -NR³C(O)NR⁴R⁵, -S(O)R³, -S(O)₂R³, -NR³S(O)R⁴, -NR³S(O)₂R⁴, -S(O)NR⁴R⁵, -S(O)₂NR⁴R⁵, or -P(O)(OR⁴)(OR⁵), wherein each R″ is, where possible, independently optionally substituted by deuterium, halogen, oxo, -OR⁶, -NR⁶R⁷, -C(O)R⁶, —CN, -S(O)R⁶, -S(O)₂R⁶, -P(O)(OR⁶)(OR⁷), C₃-C₈ cycloalkyl, 3- to 12-membered heterocyclyl, 5- to 10-membered heteroaryl, C₆-C₁₄ aryl, or C₁-C₆ alkyl optionally substituted by deuterium, oxo, —OH or halogen;
each R^{2a}, R^{2b}, R^{2c}, R^{2e}, and R^{2f} is independently oxo or R^{1a};
R^{2d} is C₁-C₆ alkyl optionally substituted by R^{2e} or C₃-C₅ cycloalkyl optionally substituted by R^{2f};
R³ is independently hydrogen, deuterium, C₁-C₆ alkyl, C₂-C₆ alkenyl, C₂-C₆ alkynyl, C₃-C₆ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 6-membered heteroaryl and 3- to 6-membered heterocyclyl of R³ are independently optionally substituted by halogen, deuterium, oxo, —CN, -OR⁸, -NR⁸R⁹, -P(O)(OR⁸)(OR⁹), or C₁-C₆ alkyl optionally substituted by deuterium, halogen, —OH or oxo;
R⁴ and R⁵ are each independently hydrogen, deuterium, C₁-C₆ alkyl, C₂-C₆ alkenyl, C₂-C₆ alkynyl, C₃-C₆ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 6-membered heteroaryl and 3- to 6-membered heterocyclyl of R⁴ and R⁵ are independently optionally substituted by deuterium, halogen, oxo, —CN, -OR⁸, -NR⁸R⁹ or C₁-C₆ alkyl optionally substituted by deuterium, halogen, —OH or oxo;
or R⁴ and R⁵ are taken together with the atom to which they attached to form a 3- to 6-membered heterocyclyl optionally substituted by deuterium, halogen, oxo, -OR⁸, -NR⁸R⁹ or C₁-C₆ alkyl optionally substituted by deuterium, halogen, oxo or —OH;
R⁶ and R⁷ are each independently hydrogen, deuterium, C₁-C₆ alkyl optionally substituted by deuterium, halogen, or oxo, C₂-C₆ alkenyl optionally substituted by deuterium, halogen, or oxo, or C₂-C₆ alkynyl optionally substituted by deuterium, halogen, or oxo;
or R⁶ and R⁷ are taken together with the atom to which they attached to form a 3- to 6-membered heterocyclyl optionally substituted by deuterium, halogen, oxo or C₁-C₆ alkyl optionally substituted by deuterium, halogen, or oxo;
R⁸ and R⁹ are each independently hydrogen, deuterium, C₁-C₆ alkyl optionally substituted by deuterium, halogen, or oxo, C₂-C₆ alkenyl optionally substituted by deuterium, halogen or oxo, or C₂-C₆ alkynyl optionally substituted by deuterium, halogen, or oxo;
or R⁸ and R⁹ are taken together with the atom to which they attached to form a 3-6 membered heterocyclyl optionally substituted by deuterium, halogen, oxo or C₁-C₆ alkyl optionally substituted by deuterium, oxo, or halogen;
each R¹⁰, R¹¹, R¹² and R¹³ are independently hydrogen or deuterium;
R¹⁴ is deuterium;
q is 0, 1, 2, 3, 4, 5, 6, 7, or 8;
each R¹⁵ is independently selected from hydrogen, deuterium, or halogen;
each R¹⁶ is independently selected from hydrogen, deuterium, or halogen; and
p is 3, 4, 5, 6, 7, 8, or 9.

In one variation is provided that the compound of Formula A excludes the free base of (2S)-4-[2-methoxyethyl-[4-(5,6,7,8-tetrahydro-1,8-naphthyridin-2-yl)butyl]amino]-2-(quinazolin-4-ylamino)butanoic acid:

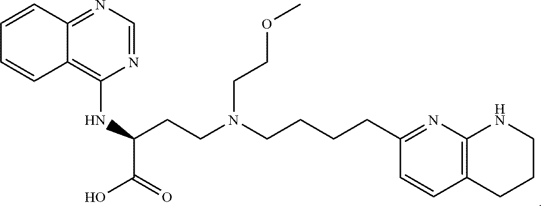

In various embodiments, the claimed compound excludes a free base of a compound represented by formula A wherein: R¹ is unsubstituted quinazolin-4-yl; R² is —CH₂CH₂OCH₃; R¹⁰, R¹¹, R¹², R¹³, R¹⁵, and R¹⁶ are each H; p is 3; q is 0; and the carbon to which R¹NH- is bonded is in the S configuration, e.g., in some embodiments, the compound of formula A excludes the free base of (2S)-4-[2-methoxyethyl-[4-(5,6,7,8-tetrahydro-1,8-naphthyridin-2-yl)butyl] amino -2-(quinazolin-4-ylamino)butanoic acid:

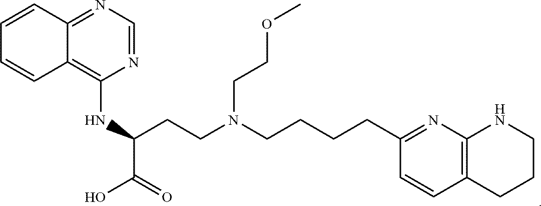

In some embodiments, the claimed compound excludes a free base of a compound represented by formula A wherein R² is —CH₂CH₂OCH₃; R¹⁰, R¹¹, R¹², R¹³, R¹⁵, and R¹⁶ are each H; p is 3; q is 0; the carbon to which R¹NH- is bonded is in the S configuration, and R¹ is one or more of the following separate lettered embodiments (a)-(k). (a) R¹ is unsubstituted quinazolin-4-yl. (b) R¹ is quinazolin-4-yl substituted by R^{1a} wherein R^{1a} is methyl. (c) R¹ is quinazolin-4-yl substituted by R^{1a} wherein R^{1a} is methyl or ethyl, (d) R¹ is quinazolin-4-yl substituted by R^{1a} wherein R^{1a} is C₁-C₆ alkyl. (e) R¹ is quinazolin-4-yl substituted by R^{1a}. (f) R¹ is a 10 membered fused bicyclic heterocycle containing two ring nitrogen atoms, and R¹ is unsubstituted or substituted by R^{1a}. (g) R¹ is unsubstituted quinazolinyl. (h) R¹ is quinazolinyl substituted by R^{1a} wherein R^{1a} is methyl. (i) R¹ is quinazolinyl substituted by R^{1a} wherein R^{1a} is methyl or ethyl, (j) R¹ is quinazolinyl substituted by R^{1a} wherein R^{1a} is C₁-C₆ alkyl, (k) R¹ is quinazolinyl substituted by R^{1a}.

In some embodiments, the claimed compound excludes a free base of a compound represented by formula A wherein R¹ is unsubstituted quinazolin-4-yl; R¹⁰, R¹¹, R¹², R¹³, R¹⁵, and R¹⁶ are each H; p is 3; q is 0; the carbon to which R¹NH- is bonded is in the S configuration, and R² is one or more of the following separate lettered embodiments (1)-(p). (1) R² is ethylene 2-substituted by R^{2a} and R^{2a} is methoxy. (m) R² is methylene, ethylene, or propylene substituted by R^{2a}, and R^{2a} is methoxy. (n) R² is ethylene substituted by R^{2a} and R^{2a} is methoxy or ethoxy. (o) R² is ethylene substituted by R^{2a} and R^{2a} is hydroxy. (p) R² is methylene, ethylene, or propylene substituted by R^{2a} and R^{2a} is hydroxy, methoxy, or ethoxy.

In some embodiments, the claimed compound excludes a free base of a compound represented by formula A wherein R¹ is unsubstituted quinazolin-4-yl; R² is -CH₂CH₂OCH₃; R¹⁵ and R¹⁶ are each H; p is 3; q is 0; the carbon to which R¹NH- is bonded is in the S configuration, and R¹⁰, R¹¹, R¹², and R¹³ together represent one or more of the following separate lettered embodiments (q)-(u). (q) Each of R¹⁰, R¹¹, R¹², and R¹³ is hydrogen. (r) One of R¹⁰, R¹¹, R¹², and R¹³ is deuterium and the rest are hydrogen. (s) Two of R¹⁰, R¹¹, R¹², and R¹³ are deuterium and the rest are hydrogen. (t) Three of R¹⁰, R¹¹, R¹², and R¹³ are deuterium and the remaining is hydrogen. (u) Each of R¹⁰, R¹¹, R¹², and R¹³ is deuterium.

In some embodiments, the claimed compound excludes a free base of a compound represented by formula A wherein R¹ is unsubstituted quinazolin-4-yl; R² is —CH₂CH₂OCH₃; R¹⁰, R¹¹, R¹², and R¹³ are each H; p is 3; q is 0; the carbon to which R¹NH- is bonded is in the S configuration, and R¹⁵ and R¹⁶ together represent one or more of the following separate lettered embodiments (v)-(aa). (v) Each of R¹⁵ and R¹⁶ is hydrogen. (w) R¹⁵ is hydrogen and R¹⁶ is deuterium, or R¹⁵ is deuterium and R¹⁶ is hydrogen. (x) R¹⁵ and R¹⁶ are deuterium, (y) R¹⁵ is hydrogen and R¹⁶ is halogen, e.g., fluorine, or R¹⁵ is halogen, e.g., fluorine, and R¹⁶ is hydrogen. (z) R¹⁵ is deuterium and R¹⁶ is halogen, e.g., fluorine, or R¹⁵ is halogen, e.g., fluorine, and R¹⁶ is deuterium, (aa) R¹⁵ and R¹⁶ are each halogen, e.g., fluorine.

In some embodiments, the claimed compound excludes a free base of a compound represented by formula A wherein R¹ is unsubstituted quinazolin-4-yl; R² is —CH₂CH₂OCH₃; R¹⁰, R¹¹, R¹², R¹³, R^{is}, and R¹⁶ are each H; q is 0; the carbon to which R¹NH- is bonded is in the S configuration; and p is one of the following separate lettered embodiments (ab)-(ad). (ab) p is 3. (ac) p is 4. (ad) p is 5.

In some embodiments, the claimed compound excludes a free base of a compound represented by formula A wherein R¹ is unsubstituted quinazolin-4-yl; R² is —CH₂CH₂OCH₃; R¹⁰, R¹¹, R¹², R¹³, R¹⁵, and R¹⁶ are each H; p is 3; the carbon to which R¹NH- is bonded is in the S configuration; and q is one of the following separate lettered embodiments (ae)-(ah). (ae) q is 0. (af) q is 1. (ag) q is 2. (ah) q is 3.

In some embodiments, excluded is a free base of a compound of any combination of the lettered embodiments selected for each of R¹; R²; R¹⁰, R¹¹, R¹², and R¹³ together; R¹⁵ and R¹⁶ together; variable p; and variable q. For example, selected may be a combination of: R¹ from one of (a)-(k); R² from one of (1)-(p); R¹⁰, R¹¹, R¹², and R¹³ together from one of (q)-(u); R¹⁵ and R¹⁶ together from one of (v)-(aa); variable p from among one of (ab)-(ad); and variable q from among one of (ae)-(ah). Exemplary combinations of lettered embodiments may include, for example: (a), (1), (q), (v), (ab), and (ae); (b), (1), (q), (v), (ab), and (ae); (c), (1), (q), (v), (ab), and (ae); (d), (1), (q), (v), (ab), and (ae); (e), (1), (q), (v), (ab), and (ae); (f), (1), (q), (v), (ab), and (ae); (g), (1), (q), (v), (ab), and (ae); (h), (1), (q), (v), (ab), and (ae); (i), (1), (q), (v), (ab), and (ae); (j), (1), (q), (v), (ab), and (ae); (k), (1), (q), (v), (ab), and (ae); (a), (m), (q), (v), (ab), and (ae); (b), (m), (q), (v), (ab), and (ae); (c), (m), (q), (v), (ab), and (ae); (d), (m), (q), (v), (ab), and (ae); (e), (m), (q), (v), (ab), and (ae); (f), (m), (q), (v), (ab), and (ae); (g), (m), (q), (v), (ab), and (ae); (h), (m), (q), (v), (ab), and (ae); (i), (m), (q), (v), (ab), and (ae); (j), (m), (q), (v), (ab), and (ae); (k), (m), (q), (v), (ab), and (ae); (a), (n), (q), (v), (ab), and (ae); (b), (n), (q), (v), (ab), and (ae); (c), (n), (q), (v), (ab), and (ae); (d), (n), (q), (v), (ab), and (ae); (e), (n), (q), (v), (ab), and (ae); (f), (n), (q), (v), (ab), and (ae); (g), (n), (q), (v), (ab), and (ae); (h), (n), (q), (v), (ab), and (ae); (i), (n), (q), (v), (ab), and (ae); (j), (n), (q), (v), (ab), and (ae); (k), (n), (q), (v), (ab), and (ae); (a), (o), (q), (v), (ab), and (ae); (b), (o), (q), (v), (ab), and (ae); (c), (o), (q), (v), (ab), and (ae); (d), (o), (q), (v), (ab), and (ae); (e), (o), (q), (v), (ab), and (ae); (f), (o), (q), (v), (ab), and (ae); (g), (o), (q), (v), (ab), and (ae); (h), (o), (q), (v), (ab), and (ae); (i), (o), (q), (v), (ab), and (ae); (j), (o), (q), (v), (ab), and (ae); (k), (o), (q), (v), (ab), and (ae); (a), (p), (q), (v), (ab), and (ae); (b), (p), (q), (v), (ab), and (ae); (c), (p), (q), (v), (ab), and (ae); (d), (p), (q), (v), (ab), and (ae); (e), (p), (q), (v), (ab), and (ae); (f), (p), (q), (v), (ab), and (ae); (g), (p), (q), (v), (ab), and (ae); (h), (p), (q), (v), (ab), and (ae); (i), (p), (q), (v), (ab), and (ae); (j), (p), (q), (v), (ab), and (ae); (k), (p), (q), (v), (ab), and (ae); any one of the preceding combinations in which (v) is replaced by (y); any one of the preceding combinations in which (v) is replaced by (aa); any one of the preceding combinations in which (ab) is replaced by (ad); or any one of the preceding combinations in which (ab) is replaced by (ae);

In some embodiments, excluded are salts of the compound of any one of, or any combination of, the lettered embodiments (a)-(ah) as described above. In some embodiments, excluded are pharmaceutical compositions that include the compound of any one of, or any combination of, the lettered embodiments (a)-(ah) as described above, or salts thereof. In some embodiments, excluded are kits that include the compound of any one of, or any combination of, the lettered embodiments (a)-(ah) as described above, or salts thereof. In some embodiments, excluded are dosage forms that include the compound of any one of, or any combination of, the lettered embodiments (a)-(ah) as described above. In some embodiments, excluded are methods that include the compound of any one of, or any combination of, the lettered embodiments (a)-(ah) as described above, or salts thereof.

In one variation is provided a compound of the formula (A), or a salt thereof, wherein the carbon bearing the CO₂H and NHR¹ moieties is in the “S” configuration. In another variation is provided a compound of the formula (A), or a salt thereof, wherein the carbon bearing the CO₂H and NHR¹ moieties is in the “R” configuration. Mixtures of a compound of the formula (A) are also embraced, including racemic or non-racemic mixtures of a given compound, and mixtures of two or more compounds of different chemical formulae.

In one variation of formula (A), R² has the proviso that any carbon atom bonded directly to a nitrogen atom is either unsubstituted or is substituted with deuterium.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ of formula (A) may be combined with every description, variation, embodiment or aspect of R² the same as if each and every combination were specifically and individually listed.

In one aspect, provided is a compound of formula (I)

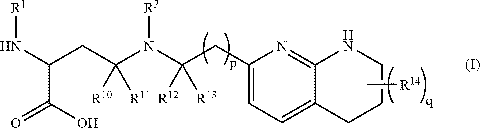

or a salt thereof, wherein:
R¹ is C₆-C₁₄ aryl or 5- to 10-membered heteroaryl wherein the C₆-C₁₄ aryl and 5- to 10-membered heteroaryl are optionally substituted by R^{1a};
R² is C₁-C₆ alkyl optionally substituted by R^{2a}; C₃-C₆ cycloalkyl optionally substituted by R^{2b}; 3- to 12-membered heterocyclyl optionally substituted by R^{2c}; or -S(O)₂R^{2d};
each R^{1a} is independently C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, C₆-C₁₄ aryl, deuterium, halogen, —CN, -OR³, -SR³, -NR⁴R⁵, —NO₂, -C=NH(OR³), -C(O)R³, -OC(O)R³, -C(O)OR³, -C(O)NR⁴R⁵, -NR³C(O)R⁴, -NR³C(O)OR⁴, -NR³C(O)NR⁴R⁵, -S(O)R³, -S(O)₂R³, -NR³S(O)R⁴, -NR³S(O)₂R⁴, -S(O)NR⁴R⁵, -S(O)₂NR⁴R⁵, or -P(O)(OR⁴)(OR⁵), wherein each R^{1a} is, where possible, independently optionally substituted by deuterium, halogen, oxo, -OR⁶, -NR⁶R⁷, -C(O)R⁶, —CN, -S(O)R⁶, -S(O)₂R⁶, -P(O)(OR⁶)(OR⁷), C₃-C₈ cycloalkyl, 3- to 12-membered heterocyclyl, 5- to 10-membered heteroaryl, C₆-C₁₄ aryl, or C₁-C₆ alkyl optionally substituted by deuterium, oxo, —OH or halogen;
each R^{2a}, R^{2b}, R^{2c}, R^{2e}, and R^{2f} is independently oxo or R^{1a};
R^{2d} is C₁-C₆ alkyl optionally substituted by R^{2e} or C₃-C₅ cycloalkyl optionally substituted by R^{2f};
R³ is independently hydrogen, deuterium, C₁-C₆ alkyl, C₂-C₆ alkenyl, C₂-C₆ alkynyl, C₃-C₆ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 6-membered heteroaryl and 3- to 6-membered heterocyclyl of R³ are independently optionally substituted by halogen, deuterium, oxo, —CN, -OR⁸, -NR⁸R⁹, -P(O)(OR⁸)(OR⁹), or C₁-C₆ alkyl optionally substituted by deuterium, halogen, —OH or oxo;
R⁴ and R⁵ are each independently hydrogen, deuterium, C₁-C₆ alkyl, C₂-C₆ alkenyl, C₂-C₆ alkynyl, C₃-C₆ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 6-membered heteroaryl and 3- to 6-membered heterocyclyl of R⁴ and R⁵ are independently optionally substituted by deuterium, halogen, oxo, —CN, -OR⁸, -NR⁸R⁹ or C₁-C₆ alkyl optionally substituted by deuterium, halogen, —OH or oxo;
or R⁴ and R⁵ are taken together with the atom to which they attached to form a 3- to 6-membered heterocyclyl optionally substituted by deuterium, halogen, oxo, -OR⁸, -NR⁸R⁹ or C₁-C₆ alkyl optionally substituted by deuterium, halogen, oxo or -OH;
R⁶ and R⁷ are each independently hydrogen, deuterium, C₁-C₆ alkyl optionally substituted by deuterium, halogen, or oxo, C₂-C₆ alkenyl optionally substituted by deuterium, halogen, or oxo, or C₂-C₆ alkynyl optionally substituted by deuterium, halogen, or oxo;
or R⁶ and R⁷ are taken together with the atom to which they attached to form a 3- to 6-membered heterocyclyl optionally substituted by deuterium, halogen, oxo or C₁-C₆ alkyl optionally substituted by deuterium, halogen, or oxo;
R⁸ and R⁹ are each independently hydrogen, deuterium, C₁-C₆ alkyl optionally substituted by deuterium, halogen, or oxo, C₂-C₆ alkenyl optionally substituted by deuterium, halogen or oxo, or C₂-C₆ alkynyl optionally substituted by deuterium, halogen, or oxo;
or R⁸ and R⁹ are taken together with the atom to which they attached to form a 3-6 membered heterocyclyl optionally substituted by deuterium, halogen, oxo or Ci-C₆ alkyl optionally substituted by deuterium, oxo, or halogen;
each R¹⁰, R¹¹, R¹², and R¹³ are independently hydrogen or deuterium;
R¹⁴ is deuterium;
q is 0, 1, 2, 3, 4, 5, 6, 7, or 8; and
p is 3, 4, 5, 6, 7, 8, or 9.

In one variation is provided a compound of the formula (I), or a salt thereof, wherein the carbon bearing the CO₂H and NHR¹ moieties is in the “S” configuration. In another variation is provided a compound of the formula (I), or a salt thereof, wherein the carbon bearing the CO₂H and NHR¹ moieties is in the “R” configuration. Mixtures of a compound of the formula (I) are also embraced, including racemic or non-racemic mixtures of a given compound, and mixtures of two or more compounds of different chemical formulae.

In one variation of formula (I), R² includes the proviso that any carbon atom bonded directly to a nitrogen atom is optionally substituted with an R^{2a} moiety other than halogen. In one variation of formula (I), R² includes the proviso that any carbon atom bonded directly to a nitrogen atom is either unsubstituted or is substituted with deuterium.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ of formula (I) may be combined with every description, variation, embodiment or aspect of R² the same as if each and every combination were specifically and individually listed.

In some embodiments of the compound of formula (I), or a salt thereof, at least one of R^{1a}, R^{2a}, R^{2b}, R^{2c}, R^{2e}, R^{2f}, R³, R⁴, R⁵, R⁶,R⁷, R⁸, R⁹, R¹⁰, R¹¹, R¹², R¹³, R¹⁴, R¹⁵, or R¹⁶ is deuterium.

In some embodiments of the compound of formula (I), or a salt thereof, R¹ is 5- to 10-membered heteroaryl optionally substituted by R^{1a}. In some embodiments, R¹ is pyrimidin-4-yl optionally substituted by R^{1a}. In some embodiments, R¹ is pyrimidin-4-yl optionally substituted by R^{1a} wherein R^{1a} is 5- to 10-membered heteroaryl (e.g., pyrazolyl) or C₁-C₆ alkyl optionally substituted by halogen (e.g., methyl, difluoromethyl, and trifluoromethyl). In some embodiments, R¹ is pyrimidin-4-yl optionally substituted by R^{1a} wherein R^{1a} is 5- to 10-membered heteroaryl (e.g., pyrazolyl or pyridinyl) or C₁-C₆ alkyl optionally substituted by halogen (e.g., methyl, difluoromethyl, and trifluoromethyl). In some embodiments, R¹ is pyrimidin-4-yl substituted by both methyl and trifluoromethyl. In some embodiments, R¹ is pyrimidin-4-yl substituted by both methyl and pyridinyl. In some embodiments, R¹ is pyrimidin-4-yl optionally substituted by R^{1a} wherein R^{1a} is C₆-C₁₄ aryl (e.g., phenyl). In some embodiments, R¹ is pyrimidin-4-yl optionally substituted by R^{1a} wherein R^{1a} is —CN. In some embodiments, R¹ is pyrimidin-2-yl optionally substituted by R^{1a}. In some embodiments, R¹ is pyrimidin-2-yl optionally substituted by R^{1a} wherein R^{1a} is halogen, C₁-C₆ alkyl optionally substituted by halogen (e.g., methyl or trifluoromethyl), —CN, or C₃-C₈ cycloalkyl (e.g., cyclopropyl). In some embodiments of the compound of formula (I), or a salt thereof, R¹ is quinazolin-4-yl optionally substituted by R^{1a}. In some embodiments, R¹ is quinazolin-4-yl optionally substituted by R^{1a} wherein R^{1a} is halogen (e.g., fluoro and chloro), C₁-C₆ alkyl optionally substituted by halogen (e.g., methyl or trifluoromethyl), or C₁-C₆ alkoxy (e.g., methoxy). In some embodiments, R¹ is quinazolin-4-yl optionally substituted by R^{1a} wherein R^{1a} is 5- to 10-membered heteroaryl (e.g., pyridinyl). In some embodiments, R¹ is pyrazolopyrimidinyl optionally substituted by R^{1a}. In some embodiments, R¹ is pyrazolopyrimidinyl optionally substituted by R^{1a}, wherein R^{1a} is C₁-C₆ alkyl (e.g., methyl). In some embodiments where R¹ is indicated as optionally substituted by R^{1a}, the R¹ moiety is unsubstituted. In some embodiments where R¹ is indicated as optionally substituted by R^{1a}, the R¹ moiety is substituted by one R^{1a}. In some embodiments where R¹ is indicated as optionally substituted by R^{1a}, the R¹ moiety is substituted by 2 to 6 or 2 to 5 or 2 to 4 or 2 to 3 R^{1a} moieties, which may be the same or different.

In some embodiments of formula (I), including the embodiments that describe the R¹ variable, each of R¹⁰, R¹¹, R¹² and R¹³ are hydrogen. In some embodiments of formula (I), including the embodiments that describe the R¹ variable, and/or the R¹⁰, R¹¹, R¹² and R¹³ variables, q is 0. In some embodiments, including the embodiments that describe the R¹ variable, and/or the R¹⁰, R¹¹, R¹² and R¹³ variables and/or the q variable, p is 3, 4 or 5.

In some embodiments of formula (I), R¹⁰, R¹¹, R¹² and R¹³ are hydrogen, p is 3, q is 0 and the compound is of the formula (II):

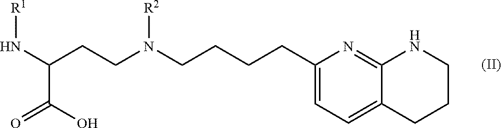

or a salt thereof, wherein R¹ and R² are as defined for formula (I).

In some embodiments of the compound of formula (I), wherein R¹ is 5- to 10-membered heteroaryl optionally substituted by R^{1a}, the compound is of the formula (I-A):

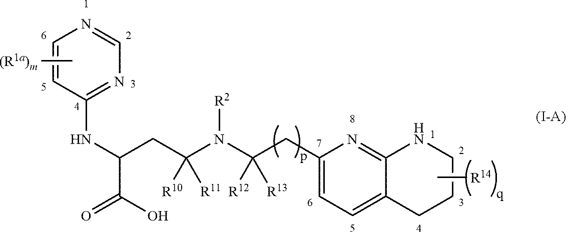

or a salt thereof, wherein R^{1a}, R², R¹⁰, R¹¹, R¹², R¹³, R¹⁴, q and p are as defined for formula (I), m is 0, 1, 2, or 3, and the positions on the pyrimidine ring and tetrahydronaphthyridine ring are as indicated.

In one embodiment is provided a compound of the formula (I-A), or a salt thereof, wherein the carbon bearing the CO₂H and NH moieties is in the “S” configuration. In another embodiment is provided a compound of the formula (I-A), or a salt thereof, wherein the carbon bearing the CO₂H and NH moieties is in the “R” configuration. Mixtures of a compound of the formula (I-A) are also embraced, including racemic or non-racemic mixtures of a given compound, and mixtures of two or more compounds of different chemical formulae.

In some embodiments of the compound of formula (I-A), m is 0, 1, 2, or 3, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I-A), m is 0, 1, 2, or 3, and each R^{1a} is, where applicable, independently deuterium, halogen, C₁-C₆ alkyl, C₁-C₆ haloalkyl (which in one variation may be C₁-C₆ perhaloalky), C₁-C₆ alkoxy, hydroxy, —CN, or 5- to 10-membered heteroaryl, wherein the C₁-C₆ alkyl, C₁-C₆ haloalkyl, C₁-C₆ alkoxy, hydroxy, and 5- to 10-membered heteroaryl of R^{1a} are independently optionally substituted by deuterium. In some embodiments of formula (I-A), m is 1, 2 or 3.

In some embodiments of the compound of formula (I-A), m is 0. In some embodiments of the compound of formula (I-A), m is 1, and R^{1a} is at the 2-position. In some embodiments of the compound of formula (I-A), m is 1, and R^{1a} is at the 5-position. In some embodiments of the compound of formula (I-A), m is 1, and R^{1a} is at the 6-position. In some embodiments of the compound of formula (I-A), m is 2, and the R^{1a} groups are at the 2-position and 5-position. In some embodiments of the compound of formula (I-A), m is 2, and the R^{1a} groups are at the 2-position and 6-position. In some embodiments of the compound of formula (I-A), m is 2, and the R^{1a} groups are at the 5-position and 6-position. In some embodiments of the compound of formula (I-A), m is 3, and the R^{1a} groups are at the 2-position, 5-position, and 6-position. Whenever more than one R^{1a} group is present, the R^{1a} groups can be chosen independently. In any of these embodiments of the compound of formula (I-A), or a salt thereof, the carbon bearing the CO₂H and NH moieties may be in the “S” configuration or the “R” configuration.

In some embodiments of formula (I-A), including the embodiments that describe the R^{1a} and m variables, each of R¹⁰, R¹¹, R¹² and R¹³ are hydrogen. In some embodiments of formula (I-A), including the embodiments that describe the R^{1a} and m variables, and/or the R¹⁰, R¹¹, R¹² and R¹³ variables, q is 0. In some embodiments of formula (I-A), including the embodiments that describe the R^{1a} and m variables, and/or the R¹⁰, R¹¹, R¹² and R¹³ variables and/or the q variable, p is 3, 4 or 5.

In some embodiments of formula (I-A), R¹⁰, R¹¹, R¹² and R¹³ are hydrogen, p is 3, q is 0 and the compound is of the formula (II-A):

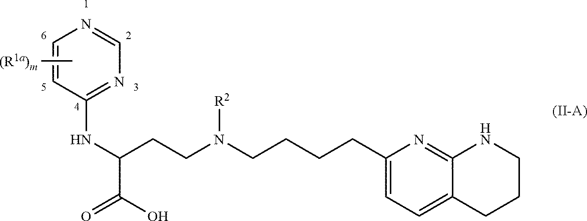

or a salt thereof, wherein R^{1a} and R² are as defined for formula (I), m is 0, 1, 2, or 3, and the positions on the pyrimidine ring are as indicated. All descriptions of R^{1a}, R² and m with reference to formula (I) apply equally to formulae (I-A) and (II-A).

In some embodiments of the compound of formula (I), wherein R¹ is 5- to 10-membered heteroaryl optionally substituted by R^{1a}, the compound is of the formula (I-B):

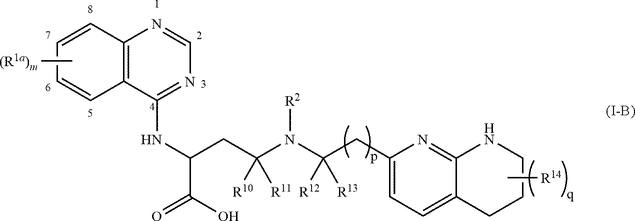

or a salt thereof, wherein R^{1a}, R², R¹⁰, R¹¹, R¹², R¹³, R¹⁴, q and p are as defined for formula (I), m is 0, 1, 2, 3, 4, or 5, and the positions on the quinazoline ring are as indicated.

In one embodiment is provided a compound of the formula (I-B), or a salt thereof, wherein the carbon bearing the CO₂H and NH moieties is in the “S” configuration. In another embodiment is provided a compound of the formula (I-B), or a salt thereof, wherein the carbon bearing the CO₂H and NH moieties is in the “R” configuration. Mixtures of a compound of the formula (I-B) are also embraced, including racemic or non-racemic mixtures of a given compound, and mixtures of two or more compounds of different chemical formulae.

In some embodiments of the compound of formula (I-B), m is 0, 1, 2, 3, 4, or 5, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I-B), m is 0, 1, 2, 3, 4, or 5, and each R^{1a} is, where applicable, independently deuterium, halogen, C₁-C₆ alkyl, C₁-C₆ haloalkyl (which in one variation may be C₁-C₆ perhaloalky), C₁-C₆ alkoxy, hydroxy, —CN, or 5- to 10-membered heteroaryl, wherein the C₁-C₆ alkyl, C₁-C₆ haloalkyl, C₁-C₆ alkoxy, hydroxy, and 5- to 10-membered heteroaryl of R^{1a} are independently optionally substituted by deuterium. In some embodiments of the compound of formula (I-B), m is 1, 2, 3, 4, or 5.

In some embodiments of the compound of formula (I-B), m is 0. In some embodiments of the compound of formula (I-B), m is 1, and R^{1a} is at the 2-position. In some embodiments of the compound of formula (I-B), m is 1, and R^{1a} is at the 5-position. In some embodiments of the compound of formula (I-B), m is 1, and R^{1a} is at the 6-position. In some embodiments of the compound of formula (I-B), m is 1, and R^{1a} is at the 7-position. In some embodiments of the compound of formula (I-B), m is 1, and R^{1a} is at the 8-position. In some embodiments of the compound of formula (I-B), m is 2, and the R^{1a} groups are at the 2-position and 5-position. In some embodiments of the compound of formula (I-B), m is 2, and the R^{1a} groups are at the 2-position and 6-position. In some embodiments of the compound of formula (I-B), m is 2, and the R^{1a} groups are at the 2-position and 7-position. In some embodiments of the compound of formula (I-B), m is 2, and the R^{1a} groups are at the 2-position and 8-position. In some embodiments of the compound of formula (I-B), m is 2, and the R^{1a} groups are at the 5-position and 6-position. In some embodiments of the compound of formula (I-B), m is 2, and the R^{1a} groups are at the 5-position and 7-position. In some embodiments of the compound of formula (I-B), m is 2, and the R^{1a} groups are at the 5-position and 8-position. In some embodiments of the compound of formula (I-B), m is 2, and the R^{1a} groups are at the 6-position and 7-position. In some embodiments of the compound of formula (I-B), m is 2, and the R^{1a} groups are at the 6-position and 8-position. In some embodiments of the compound of formula (I-B), m is 2, and the R^{1a} groups are at the 7-position and 8-position. In some embodiments of the compound of formula (I-B), m is 3, and the R^{1a} groups are at the 2-position, 5-position, and 6-position. In some embodiments of the compound of formula (I-B), m is 3, and the R^{1a} groups are at the 2-position, 5-position, and 7-position. In some embodiments of the compound of formula (I-B), m is 3, and the R^{1a} groups are at the 2-position, 5-position, and 8-position. In some embodiments of the compound of formula (I-B), m is 3, and the R^{1a} groups are at the 2-position, 6-position, and 7-position. In some embodiments of the compound of formula (I-B), m is 3, and the R^{1a} groups are at the 2-position, 6-position, and 8-position. In some embodiments of the compound of formula (I-B), m is 3, and the R^{1a} groups are at the 2-position, 7-position, and 8-position. In some embodiments of the compound of formula (I-B), m is 3, and the R^{1a} groups are at the 5-position, 6-position, and 7-position. In some embodiments of the compound of formula (I-B), m is 3, and the R^{1a} groups are at the 5-position, 6-position, and 8-position. In some embodiments of the compound of formula (I-B), m is 3, and the R^{1a} groups are at the 5-position, 7-position, and 8-position. In some embodiments of the compound of formula (I-B), m is 3, and the R^{1a} groups are at the 6-position, 7-position, and 8-position. In some embodiments of the compound of formula (I-B), m is 4, and the R^{1a} groups are at the 2-position, 5-position, 6-position, and 7-position. In some embodiments of the compound of formula (I-B), m is 4, and the R^{1a} groups are at the 2-position, 5-position, 6-position, and 8-position. In some embodiments of the compound of formula (I-B), m is 4, and the R^{1a} groups are at the 2-position, 5-position, 7-position, and 8-position. In some embodiments of the compound of formula (I-B), m is 4, and the R^{1a} groups are at the 2-position, 6-position, 7-position, and 8-position. In some embodiments of the compound of formula (I-B), m is 4, and the R^{1a} groups are at the 5-position, 6-position, 7-position, and 8-position. In some embodiments of the compound of formula (I-B), m is 5, and the R^{1a} groups are at the 2-position, 5-position, 6-position, 7-position, and 8-position. Whenever more than one R^{1a} group is present, the R^{1a} groups can be chosen independently. In any of these embodiments of the compound of formula (I-B), or a salt thereof, the carbon bearing the CO₂H and NH moieties may be in the “S” configuration or the “R” configuration.

In some embodiments of formula (I-B), including the embodiments that describe the R^{1a} and m variables, each of R¹⁰, R¹¹, R¹² and R¹³ are hydrogen. In some embodiments of formula (I-B), including the embodiments that describe the R^{1a} and m variables, and/or the R¹⁰, R¹¹, R¹² and R¹³ variables, q is 0. In some embodiments of formula (I-B), including the embodiments that describe the R^{1a} and m variables, and/or the R¹⁰, R¹¹, R¹² and R¹³ variables and/or the q variable, p is 3, 4 or 5.

In some embodiments of formula (I-B), R¹⁰, R¹¹, R¹² and R¹³ are hydrogen, p is 3, q is 0 and the compound is of the formula (II-B):

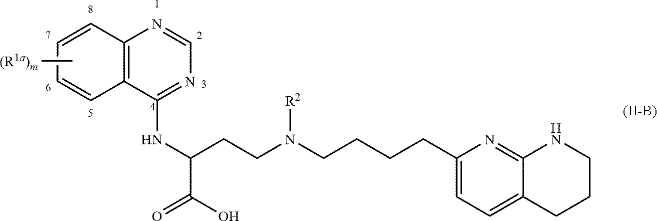

or a salt thereof, wherein R^{1a} and R² are as defined for formula (I), m is 0, 1, 2, 3, 4, or 5, and the positions on the quinazoline ring are as indicated. All descriptions of R^{1a}, R² and m with reference to formula (I) apply equally to formulae (I-B) and (II-B).

In some embodiments of the compound of formula (I), wherein R¹ is 5- to 10-membered heteroaryl optionally substituted by R^{1a}, the compound is of the formula (I-C):

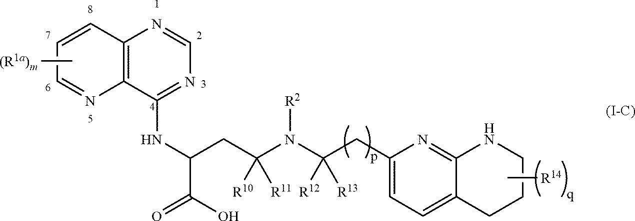

or a salt thereof, wherein R^{1a}, R², R¹⁰, R¹¹, R¹², R¹³, R¹⁴, q and p are as defined for formula (I), m is 0, 1, 2, 3, or 4, and the positions on the pyrido[3,2-d]pyrimidine ring are as indicated. In one embodiment is provided a compound of the formula (I-C), or a salt thereof, wherein the carbon bearing the CO₂H and NH moieties is in the “S” configuration. In another embodiment is provided a compound of the formula (I-C), or a salt thereof, wherein the carbon bearing the CO₂H and NH moieties is in the “R” configuration. Mixtures of a compound of the formula (I-C) are also embraced, including racemic or non-racemic mixtures of a given compound, and mixtures of two or more compounds of different chemical formulae.

In some embodiments of the compound of formula (I-C), m is 0, 1, 2, 3, or 4, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I-C), m is 0, 1, 2, 3, or 4, and each R^{1a} is, where applicable, independently deuterium, halogen, C₁-C₆ alkyl, C₁-C₆ haloalkyl (which in one variation may be C₁-C₆ perhaloalky), C₁-C₆ alkoxy, hydroxy, —CN, or 5- to 10-membered heteroaryl, wherein the C₁-C₆ alkyl, C₁-C₆ haloalkyl, C₁-C₆ alkoxy, hydroxy, and 5- to 10-membered heteroaryl of R^{1a} are independently optionally substituted by deuterium. In some embodiments of the compound of formula (I-C), m is 1, 2, 3, or 4

In some embodiments of the compound of formula (I-C), m is 0. In some embodiments of the compound of formula (I-C), m is 1, and R^{1a} is at the 2-position. In some embodiments of the compound of formula (I-C), m is 1, and R^{1a} is at the 6-position. In some embodiments of the compound of formula (I-C), m is 1, and R^{1a} is at the 7-position. In some embodiments of the compound of formula (I-C), m is 1, and R^{1a} is at the 8-position. In some embodiments of the compound of formula (I-C), m is 2, and the R^{1a} groups are at the 2-position and 6-position. In some embodiments of the compound of formula (I-C), m is 2, and the R^{1a} groups are at the 2-position and 7-position. In some embodiments of the compound of formula (I-C), m is 2, and the R^{1a} groups are at the 2-position and 8-position. In some embodiments of the compound of formula (I-C), m is 2, and the R^{1a} groups are at the 6-position and 7-position. In some embodiments of the compound of formula (I-C), m is 2, and the R^{1a} groups are at the 6-position and 8-position. In some embodiments of the compound of formula (I-C), m is 2, and the R^{1a} groups are at the 7-position and 8-position. In some embodiments of the compound of formula (I-C), m is 3, and the R^{1a} groups are at the 2-position, 6-position, and 7-position. In some embodiments of the compound of formula (I-C), m is 3, and the R^{1a} groups are at the 2-position, 6-position, and 8-position. In some embodiments of the compound of formula (I-C), m is 3, and the R^{1a} groups are at the 2-position, 7-position, and 8-position. In some embodiments of the compound of formula (I-C), m is 3, and the R^{1a} groups are at the 6-position, 7-position, and 8-position. In some embodiments of the compound of formula (I-C), m is 4, and the R^{1a} groups are at the 2-position, 6-position, 7-position, and 8-position. Whenever more than one R^{1a} group is present, the R^{1a} groups can be chosen independently. In any of these embodiments of the compound of formula (I-C), or a salt thereof, the carbon bearing the CO₂H and NH moieties may be in the “S” configuration or the “R” configuration.

In some embodiments of formula (I-C), including the embodiments that describe the R^{1a} and m variables, each of R¹⁰, R¹¹, R¹² and R¹³ are hydrogen. In some embodiments of formula (I-C), including the embodiments that describe the R^{1a} and m variables, and/or the R¹⁰, R¹¹, R¹² and R¹³ variables, q is 0. In some embodiments of formula (I-C), including the embodiments that describe the R^{1a} and m variables, and/or the R¹⁰, R¹¹, R¹² and R¹³ variables and/or the q variable, p is 3, 4 or 5.

In some embodiments of formula (I-C), R¹⁰, R¹¹, R¹² and R¹³ are hydrogen, p is 3, q is 0 and the compound is of the formula (II-C):

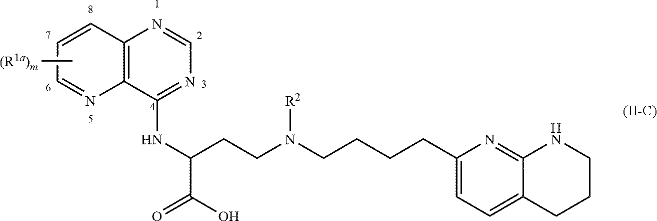

or a salt thereof, wherein R^{1a} and R² are as defined for formula (I), m is 0, 1, 2, 3, or 4, and the positions on the pyrido[3,2-d]pyrimidine ring are as indicated. All descriptions of R^{1a}, R² and m with reference to formula (I) apply equally to formulae (I-C) and (II-C).

In some embodiments of the compound of formula (I), wherein R¹ is 5- to 10-membered heteroaryl optionally substituted by R^{1a}, the compound is of the formula (I-D):

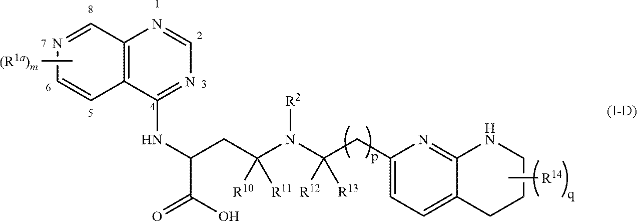

or a salt thereof, wherein R^{1a}, R², R¹⁰, R¹¹, R¹², R¹³, R¹⁴, q and p are as defined for formula (I), m is 0, 1, 2, 3, or 4, and the positions on the pyrido[3,4-d]pyrimidine ring are as indicated.

In one embodiment is provided a compound of the formula (I-D), or a salt thereof, wherein the carbon bearing the CO₂H and NH moieties is in the “S” configuration. In another embodiment is provided a compound of the formula (I-D), or a salt thereof, wherein the carbon bearing the CO₂H and NH moieties is in the “R” configuration. Mixtures of a compound of the formula (I-D) are also embraced, including racemic or non-racemic mixtures of a given compound, and mixtures of two or more compounds of different chemical formulae.

In some embodiments of the compound of formula (I-D), m is 0, 1, 2, 3, or 4, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I-D), m is 0, 1, 2, 3, or 4, and each R^{1a} is, where applicable, independently deuterium, halogen, C₁-C₆ alkyl, C₁-C₆ haloalkyl (which in one variation may be C₁-C₆ perhaloalky), C₁-C₆ alkoxy, hydroxy, —CN, or 5- to 10-membered heteroaryl, wherein the C₁-C₆ alkyl, C₁-C₆ haloalkyl, C₁-C₆ alkoxy, hydroxy, and 5- to 10-membered heteroaryl of R^{1a} are independently optionally substituted by deuterium. In some embodiments of the compound of formula (I-D), m is 1, 2, 3, or 4.

In some embodiments of the compound of formula (I-D), m is 0. In some embodiments of the compound of formula (I-D), m is 1, and R^{1a} is at the 2-position. In some embodiments of the compound of formula (I-D), m is 1, and R^{1a} is at the 5-position. In some embodiments of the compound of formula (I-D), m is 1, and R^{1a} is at the 6-position. In some embodiments of the compound of formula (I-D), m is 1, and R^{1a} is at the 8-position. In some embodiments of the compound of formula (I-D), m is 2, and the R^{1a} groups are at the 2-position and 5-position. In some embodiments of the compound of formula (I-D), m is 2, and the R^{1a} groups are at the 2-position and 6-position. In some embodiments of the compound of formula (I-D), m is 2, and the R^{1a} groups are at the 2-position and 8-position. In some embodiments of the compound of formula (I-D), m is 2, and the R^{1a} groups are at the 5-position and 6-position. In some embodiments of the compound of formula (I-D), m is 2, and the R^{1a} groups are at the 5-position and 8-position. In some embodiments of the compound of formula (I-D), m is 2, and the R^{1a} groups are at the 6-position and 8-position. In some embodiments of the compound of formula (I-D), m is 3, and the R^{1a} groups are at the 2-position, 5-position, and 6-position. In some embodiments of the compound of formula (I-D), m is 3, and the R^{1a} groups are at the 2-position, 5-position, and 8-position. In some embodiments of the compound of formula (I-D), m is 3, and the R^{1a} groups are at the 2-position, 6-position, and 8-position. In some embodiments of the compound of formula (I-D), m is 3, and the R^{1a} groups are at the 5-position, 6-position, and 8-position. In some embodiments of the compound of formula (I-D), m is 4, and the R^{1a} groups are at the 2-position, 5-position, 6-position, and 8-position. Whenever more than one R^{1a} group is present, the R^{1a} groups can be chosen independently. In any of these embodiments of the compound of formula (I-D), or a salt thereof, the carbon bearing the CO₂H and NH moieties may be in the “S” configuration or the “R” configuration.

In some embodiments of formula (I-D), including the embodiments that describe the R^{1a} and m variables, each of R¹⁰, R¹¹, R¹² and R¹³ are hydrogen. In some embodiments of formula (I-D), including the embodiments that describe the R^{1a} and m variables, and/or the R¹⁰, R¹¹, R¹² and R¹³ variables, q is 0. In some embodiments of formula (I-D), including the embodiments that describe the R^{1a} and m variables, and/or the R¹⁰, R¹¹, R¹² and R¹³ variables and/or the q variable, p is 3, 4 or 5.

In some embodiments of formula (I-D), R¹⁰, R¹¹, R¹² and R¹³ are hydrogen, p is 3, q is 0 and the compound is of the formula (II-D):

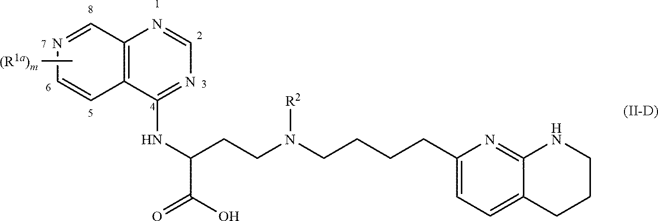

or a salt thereof, wherein R^{1a} and R² are as defined for formula (I), m is 0, 1, 2, 3, or 4, and the positions on the pyrido[3,4-d]pyrimidine ring are as indicated. All descriptions of R^{1a}, R² and m with reference to formula (I) apply equally to formulae (I-D) and (II-D).

In some embodiments of the compound of formula (I), wherein R¹ is 5- to 10-membered heteroaryl optionally substituted by R^{1a}, the compound is of the formula (I-E):

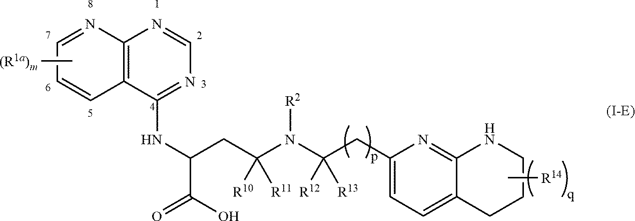

or a salt thereof, wherein R^{1a}, R², R¹⁰, R¹¹, R¹², R¹³, R¹⁴, q and p are as defined for formula (I), m is 0, 1, 2, 3, or 4, and the positions on the pyrido[2,3-d]pyrimidine ring are as indicated.

In one embodiment is provided a compound of the formula (I-E), or a salt thereof, wherein the carbon bearing the CO₂H and NH moieties is in the “S” configuration. In another embodiment is provided a compound of the formula (I-E), or a salt thereof, wherein the carbon bearing the CO₂H and NH moieties is in the “R” configuration. Mixtures of a compound of the formula (I-E) are also embraced, including racemic or non-racemic mixtures of a given compound, and mixtures of two or more compounds of different chemical formulae.

In some embodiments of the compound of formula (I-E), m is 0, 1, 2, 3, or 4, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I-E), m is 0, 1, 2, 3, or 4, and each R^{1a} is, where applicable, independently deuterium, halogen, C₁-C₆ alkyl, C₁-C₆ haloalkyl (which in one variation may be C₁-C₆ perhaloalky), C₁-C₆ alkoxy, hydroxy, —CN, or 5- to 10-membered heteroaryl, wherein the C₁-C₆ alkyl, C₁-C₆ haloalkyl, C₁-C₆ alkoxy, hydroxy, and 5- to 10-membered heteroaryl of R^{1a} are independently optionally substituted by deuterium. In some embodiments of the compound of formula (I-E), m is 1, 2, 3, or 4.

In some embodiments of the compound of formula (I-E), m is 0. In some embodiments of the compound of formula (I-E), m is 1, and R^{1a} is at the 2-position. In some embodiments of the compound of formula (I-E), m is 1, and R^{1a} is at the 5-position. In some embodiments of the compound of formula (I-E), m is 1, and R^{1a} is at the 6-position. In some embodiments of the compound of formula (I-E), m is 1, and R^{1a} is at the 7-position. In some embodiments of the compound of formula (I-E), m is 2, and the R^{1a} groups are at the 2-position and 5-position. In some embodiments of the compound of formula (I-E), m is 2, and the R^{1a} groups are at the 2-position and 6-position. In some embodiments of the compound of formula (I-E), m is 2, and the R^{1a} groups are at the 2-position and 7-position. In some embodiments of the compound of formula (I-E), m is 2, and the R^{1a} groups are at the 5-position and 6-position. In some embodiments of the compound of formula (I-E), m is 2, and the R^{1a} groups are at the 5-position and 7-position. In some embodiments of the compound of formula (I-E), m is 2, and the R^{1a} groups are at the 6-position and 7-position. In some embodiments of the compound of formula (I-E), m is 3, and the R^{1a} groups are at the 2-position, 5-position, and 6-position. In some embodiments of the compound of formula (I-E), m is 3, and the R^{1a} groups are at the 2-position, 5-position, and 7-position. In some embodiments of the compound of formula (I-E), m is 3, and the R^{1a} groups are at the 2-position, 6-position, and 7-position. In some embodiments of the compound of formula (I-E), m is 3, and the R^{1a} groups are at the 5-position, 6-position, and 7-position. In some embodiments of the compound of formula (I-E), m is 4, and the R^{1a} groups are at the 2-position, 5-position, 6-position, and 7-position. Whenever more than one R^{1a} group is present, the R^{1a} groups can be chosen independently. In any of these embodiments of the compound of formula (I-E), or a salt thereof, the carbon bearing the CO₂H and NH moieties may be in the “S” configuration or the “R” configuration.

In some embodiments of formula (I-E), including the embodiments that describe the R^{1a} and m variables, each of R¹⁰, R¹¹, R¹² and R¹³ are hydrogen. In some embodiments of formula (I-E), including the embodiments that describe the R^{1a} and m variables, and/or the R¹⁰, R¹¹, R¹² and R¹³ variables, q is 0. In some embodiments of formula (I-E), including the embodiments that describe the R^{1a} and m variables, and/or the R¹⁰, R¹¹, R¹² and R¹³ variables and/or the q variable, p is 3, 4 or 5.

In some embodiments of formula (I-E), R¹⁰, R¹¹, R¹² and R¹³ are hydrogen, p is 3, q is 0 and the compound is of the formula (II-E):

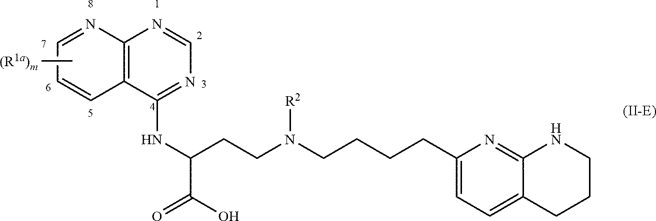

or a salt thereof, wherein R^{1a} and R² are as defined for formula (I), m is 0, 1, 2, 3, or 4, and the positions on the pyrido[2,3-d]pyrimidine ring are as indicated. All descriptions of R^{1a}, R² and m with reference to formula (I) apply equally to formulae (I-E) and (II-E).

In some embodiments of the compound of formula (I), wherein R¹ is 5- to 10-membered heteroaryl optionally substituted by R^{1a}, the compound is of the formula (I-F):

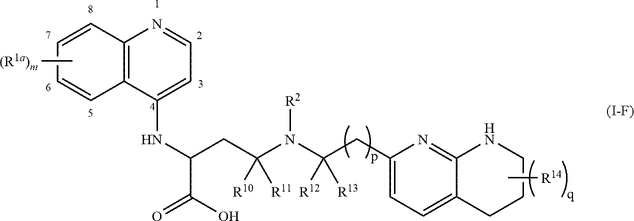

or a salt thereof, wherein R^{1a}, R², R¹⁰, R¹¹, R¹², R¹³, R¹⁴, q and p are as defined for formula (I), m is 0, 1, 2, 3, 4, 5, or 6 and the positions on the quinoline ring are as indicated.

In one embodiment is provided a compound of the formula (I-F), or a salt thereof, wherein the carbon bearing the CO₂H and NH moieties is in the “S” configuration. In another embodiment is provided a compound of the formula (I-F), or a salt thereof, wherein the carbon bearing the CO₂H and NH moieties is in the “R” configuration. Mixtures of a compound of the formula (I-F) are also embraced, including racemic or non-racemic mixtures of a given compound, and mixtures of two or more compounds of different chemical formulae.

In some embodiments of the compound of formula (I-F), m is 0, 1, 2, 3, 4, 5, or 6 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I-F), m is 0, 1, 2, 3, 4, 5, or 6, and each R^{1a} is, where applicable, independently deuterium, halogen, C₁-C₆ alkyl, C₁-C₆ haloalkyl (which in one variation may be C₁-C₆ perhaloalky), C₁-C₆ alkoxy, hydroxy, —CN, or 5- to 10-membered heteroaryl, wherein the C₁-C₆ alkyl, C₁-C₆ haloalkyl, C₁-C₆ alkoxy, hydroxy, and 5- to 10-membered heteroaryl of R^{1a} are independently optionally substituted by deuterium. In some embodiments of the compound of formula (I-F), m is 1, 2, 3, 4, 5, or 6.

In some embodiments of the compound of formula (I-F), m is 0. In some embodiments of the compound of formula (I-F), m is 1, and R^{1a} is at the 2-position. In some embodiments of the compound of formula (I-F), m is 1, and R^{1a} is at the 3-position. In some embodiments of the compound of formula (I-F), m is 1, and R^{1a} is at the 5-position. In some embodiments of the compound of formula (I-F), m is 1, and R^{1a} is at the 6-position. In some embodiments of the compound of formula (I-F), m is 1, and R^{1a} is at the 7-position. In some embodiments of the compound of formula (I-F), m is 1, and R^{1a} is at the 8-position. In some embodiments of the compound of formula (I-F), m is 2, and the R^{1a} groups are at the 2-position and 3-position. In some embodiments of the compound of formula (I-F), m is 2, and the R^{1a} groups are at the 2-position and 5-position. In some embodiments of the compound of formula (I-F), m is 2, and the R^{1a} groups are at the 2-position and 6-position. In some embodiments of the compound of formula (I-F), m is 2, and the R^{1a} groups are at the 2-position and 7-position. In some embodiments of the compound of formula (I-F), m is 2, and the R^{1a} groups are at the 2-position and 8-position. In some embodiments of the compound of formula (I-F), m is 2, and the R^{1a} groups are at the 3-position and 5-position. In some embodiments of the compound of formula (I-F), m is 2, and the R^{1a} groups are at the 3-position and 6-position. In some embodiments of the compound of formula (I-F), m is 2, and the R^{1a} groups are at the 3-position and 7-position. In some embodiments of the compound of formula (I-F), m is 2, and the R^{1a} groups are at the 3-position and 8-position.In some embodiments of the compound of formula (I-F), m is 2, and the R^{1a} groups are at the 5-position and 6-position. In some embodiments of the compound of formula (I-F), m is 2, and the R^{1a} groups are at the 5-position and 7-position. In some embodiments of the compound of formula (I-F), m is 2, and the R^{1a} groups are at the 5-position and 8-position. In some embodiments of the compound of formula (I-F), m is 2, and the R^{1a} groups are at the 6-position and 7-position. In some embodiments of the compound of formula (I-F), m is 2, and the R^{1a} groups are at the 6-position and 8-position. In some embodiments of the compound of formula (I-F), m is 2, and the R^{1a} groups are at the 7-position and 8-position. In some embodiments of the compound of formula (I-F), m is 3, and the R^{1a} groups are at the 2-position, 3-position, and 5-position. In some embodiments of the compound of formula (I-F), m is 3, and the R^{1a} groups are at the 2-position, 3-position, and 6-position. In some embodiments of the compound of formula (I-F), m is 3, and the R^{1a} groups are at the 2-position, 3-position, and 7-position. In some embodiments of the compound of formula (I-F), m is 3, and the R^{1a} groups are at the 2-position, 3-position, and 8-position. In some embodiments of the compound of formula (I-F), m is 3, and the R^{1a} groups are at the 2-position, 5-position, and 6-position. In some embodiments of the compound of formula (I-F), m is 3, and the R^{1a} groups are at the 2-position, 5-position, and 7-position. In some embodiments of the compound of formula (I-F), m is 3, and the R^{1a} groups are at the 2-position, 5-position, and 8-position. In some embodiments of the compound of formula (I-F), m is 3, and the R^{1a} groups are at the 2-position, 6-position, and 7-position. In some embodiments of the compound of formula (I-F), m is 3, and the R^{1a} groups are at the 2-position, 6-position, and 8-position. In some embodiments of the compound of formula (I-F), m is 3, and the R^{1a} groups are at the 2-position, 7-position, and 8-position. In some embodiments of the compound of formula (I-F), m is 3, and the R^{1a} groups are at the 3-position, 5-position, and 6-position. In some embodiments of the compound of formula (I-F), m is 3, and the R^{1a} groups are at the 3-position, 5-position, and 7-position. In some embodiments of the compound of formula (I-F), m is 3, and the R^{1a} groups are at the 3-position, 5-position, and 8-position. In some embodiments of the compound of formula (I-F), m is 3, and the R^{1a} groups are at the 3-position, 6-position, and 7-position. In some embodiments of the compound of formula (I-F), m is 3, and the R^{1a} groups are at the 3-position, 6-position, and 8-position. In some embodiments of the compound of formula (I-F), m is 3, and the R^{1a} groups are at the 3-position, 7-position, and 8-position. In some embodiments of the compound of formula (I-F), m is 3, and the R^{1a} groups are at the 5-position, 6-position, and 7-position. In some embodiments of the compound of formula (I-F), m is 3, and the R^{1a} groups are at the 5-position, 6-position, and 8-position. In some embodiments of the compound of formula (I-F), m is 3, and the R^{1a} groups are at the 5-position, 7-position, and 8-position. In some embodiments of the compound of formula (I-F), m is 3, and the R^{1a} groups are at the 6-position, 7-position, and 8-position. In some embodiments of the compound of formula (I-F), m is 4, and the R^{1a} groups are at the 2-position, 3-position, 5-position, and 6-position. In some embodiments of the compound of formula (I-F), m is 4, and the R^{1a} groups are at the 2-position, 3-position, 5-position, and 7-position. In some embodiments of the compound of formula (I-F), m is 4, and the R^{1a} groups are at the 2-position, 3-position, 5-position, and 8-position. In some embodiments of the compound of formula (I-F), m is 4, and the R^{1a} groups are at the 2-position, 3-position, 6-position, and 7-position. In some embodiments of the compound of formula (I-F), m is 4, and the R^{1a} groups are at the 2-position, 3-position, 6-position, and 8-position. In some embodiments of the compound of formula (I-F), m is 4, and the R^{1a} groups are at the 2-position, 3-position, 7-position, and 8-position. In some embodiments of the compound of formula (I-F), m is 4, and the R^{1a} groups are at the 2-position, 5-position, 6-position, and 7-position. In some embodiments of the compound of formula (I-F), m is 4, and the R^{1a} groups are at the 2-position, 5-position, 6-position, and 8-position. In some embodiments of the compound of formula (I-F), m is 4, and the R^{1a} groups are at the 2-position, 5-position, 7-position, and 8-position. In some embodiments of the compound of formula (I-F), m is 4, and the R^{1a} groups are at the 2-position, 6-position, 7-position, and 8-position. In some embodiments of the compound of formula (I-F), m is 4, and the R^{1a} groups are at the 3-position, 5-position, 6-position, and 7-position. In some embodiments of the compound of formula (I-F), m is 4, and the R^{1a} groups are at the 3-position, 5-position, 6-position, and 8-position. In some embodiments of the compound of formula (I-F), m is 4, and the R^{1a} groups are at the 3-position, 5-position, 7-position, and 8-position. In some embodiments of the compound of formula (I-F), m is 4, and the R^{1a} groups are at the 3-position, 6-position, 7-position, and 8-position.In some embodiments of the compound of formula (I-F), m is 4, and the R^{1a} groups are at the 5-position, 6-position, 7-position, and 8-position. In some embodiments of the compound of formula (I-F), m is 5, and the R^{1a} groups are at the 2-position, 3-position, 5-position, 6-position, and 7-position. In some embodiments of the compound of formula (I-F), m is 5, and the R^{1a} groups are at the 2-position, 3-position, 5-position, 6-position, and 8-position. In some embodiments of the compound of formula (I-F), m is 5, and the R^{1a} groups are at the 2-position, 3-position, 5-position, 7-position, and 8-position. In some embodiments of the compound of formula (I-F), m is 5, and the R^{1a} groups are at the 2-position, 3-position, 6-position, 7-position, and 8-position. In some embodiments of the compound of formula (I-F), m is 5, and the R^{1a} groups are at the 2-position, 5-position, 6-position, 7-position, and 8-position. In some embodiments of the compound of formula (IF), m is 5, and the R^{1a} groups are at the 3-position, 5-position, 6-position, 7-position, and 8-position. In some embodiments of the compound of formula (I-F), m is 6, and the R^{1a} groups are at the 2-position, 3-position, 5-position, 6-position, 7-position, and 8-position. Whenever more than one R^{1a} group is present, the R^{1a} groups can be chosen independently. In any of these embodiments of the compound of formula (I-F), or a salt thereof, the carbon bearing the CO₂H and NH moieties may be in the “S” configuration or the “R” configuration.

In some embodiments of formula (I-F), including the embodiments that describe the R^{1a} and m variables, each of R¹⁰, R¹¹, R¹² and R¹³ are hydrogen. In some embodiments of formula (I-F), including the embodiments that describe the R^{1a} and m variables, and/or the R¹⁰, R¹¹, R¹² and R¹³ variables, q is 0. In some embodiments of formula (I-F), including the embodiments that describe the R^{1a} and m variables, and/or the R¹⁰, R¹¹, R¹² and R¹³ variables and/or the q variable, p is 3, 4 or 5.

In some embodiments of formula (I-F), R¹⁰, R¹¹, R¹² and R¹³ are hydrogen, p is 3, q is 0 and the compound is of the formula (II-F):

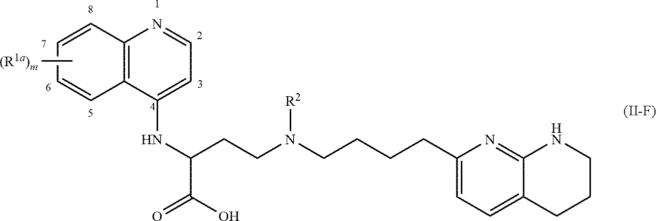

or a salt thereof, wherein R^{1a} and R² are as defined for formula (I), m is 0, 1, 2, 3, 4, 5, or 6 and the positions on the quinoline ring are as indicated. All descriptions of R^{1a}, R² and m with reference to formula (I) apply equally to formulae (I-F) and (II-F).

In some embodiments of the compound of formula (I), wherein R¹ is 5- to 10-membered heteroaryl optionally substituted by R^{1a}, the compound is of the formula (I-G):

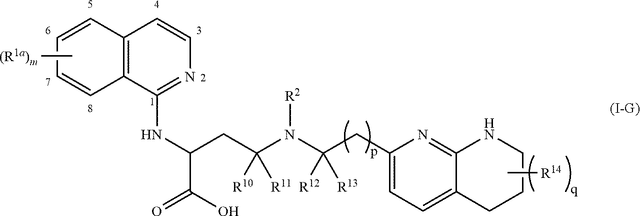

or a salt thereof, wherein R^{1a}, R², R¹⁰, R¹¹, R¹², R¹³, R¹⁴, q and p are as defined for formula (I), m is 0, 1, 2, 3, 4, 5, or 6 and the positions on the isoquinoline ring are as indicated.

In one embodiment is provided a compound of the formula (I-G), or a salt thereof, wherein the carbon bearing the CO₂H and NH moieties is in the “S” configuration. In another embodiment is provided a compound of the formula (I-G), or a salt thereof, wherein the carbon bearing the CO₂H and NH moieties is in the “R” configuration. Mixtures of a compound of the formula (I-G) are also embraced, including racemic or non-racemic mixtures of a given compound, and mixtures of two or more compounds of different chemical formulae.

In some embodiments of the compound of formula (I-G), m is 0, 1, 2, 3, 4, 5, or 6 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I-G), m is 0, 1, 2, 3, 4, 5, or 6, and each R^{1a} is, where applicable, independently deuterium, halogen, C₁-C₆ alkyl, C₁-C₆ haloalkyl (which in one variation may be C₁-C₆ perhaloalky), C₁-C₆ alkoxy, hydroxy, —CN, or 5- to 10-membered heteroaryl, wherein the C₁-C₆ alkyl, C₁-C₆ haloalkyl, C₁-C₆ alkoxy, hydroxy, and 5- to 10-membered heteroaryl of R^{1a} are independently optionally substituted by deuterium. In some embodiments of the compound of formula (I-G), m is 1, 2, 3, 4, 5, or 6.

In some embodiments of the compound of formula (I-G), m is 0. In some embodiments of the compound of formula (I-G), m is 1, and R^{1a} is at the 3-position. In some embodiments of the compound of formula (I-G), m is 1, and R^{1a} is at the 4-position. In some embodiments of the compound of formula (I-G), m is 1, and R^{1a} is at the 5-position. In some embodiments of the compound of formula (I-G), m is 1, and R^{1a} is at the 6-position. In some embodiments of the compound of formula (I-G), m is 1, and R^{1a} is at the 7-position. In some embodiments of the compound of formula (I-G), m is 1, and R^{1a} is at the 8-position. In some embodiments of the compound of formula (I-G), m is 2, and the R^{1a} groups are at the 3-position and 4-position. In some embodiments of the compound of formula (I-G), m is 2, and the R^{1a} groups are at the 4-position and 5-position. In some embodiments of the compound of formula (I-G), m is 2, and the R^{1a} groups are at the 4-position and 6-position. In some embodiments of the compound of formula (I-G), m is 2, and the R^{1a} groups are at the 4-position and 7-position. In some embodiments of the compound of formula (I-G), m is 2, and the R^{1a} groups are at the 4-position and 8-position. In some embodiments of the compound of formula (I-G), m is 2, and the R^{1a} groups are at the 3-position and 5-position. In some embodiments of the compound of formula (I-G), m is 2, and the R^{1a} groups are at the 3-position and 6-position. In some embodiments of the compound of formula (I-G), m is 2, and the R^{1a} groups are at the 3-position and 7-position. In some embodiments of the compound of formula (I-G), m is 2, and the R^{1a} groups are at the 3-position and 8-position.In some embodiments of the compound of formula (I-G), m is 2, and the R^{1a} groups are at the 5-position and 6-position. In some embodiments of the compound of formula (I-G), m is 2, and the R^{1a} groups are at the 5-position and 7-position. In some embodiments of the compound of formula (I-G), m is 2, and the R^{1a} groups are at the 5-position and 8-position. In some embodiments of the compound of formula (I-G), m is 2, and the R^{1a} groups are at the 6-position and 7-position. In some embodiments of the compound of formula (I-G), m is 2, and the R^{1a} groups are at the 6-position and 8-position. In some embodiments of the compound of formula (I-G), m is 2, and the R^{1a} groups are at the 7-position and 8-position. In some embodiments of the compound of formula (I-G), m is 3, and the R^{1a} groups are at the 3-position, 4-position, and 5-position. In some embodiments of the compound of formula (I-G), m is 3, and the R^{1a} groups are at the 3-position, 4-position, and 6-position. In some embodiments of the compound of formula (I-G), m is 3, and the R^{1a} groups are at the 3-position, 4-position, and 7-position. In some embodiments of the compound of formula (I-G), m is 3, and the R^{1a} groups are at the 3-position, 4-position, and 8-position. In some embodiments of the compound of formula (I-G), m is 3, and the R^{1a} groups are at the 4-position, 5-position, and 6-position. In some embodiments of the compound of formula (I-G), m is 3, and the R^{1a} groups are at the 4-position, 5-position, and 7-position. In some embodiments of the compound of formula (I-G), m is 3, and the R^{1a} groups are at the 4-position, 5-position, and 8-position. In some embodiments of the compound of formula (I-G), m is 3, and the R^{1a} groups are at the 4-position, 6-position, and 7-position. In some embodiments of the compound of formula (I-G), m is 3, and the R^{1a} groups are at the 4-position, 6-position, and 8-position. In some embodiments of the compound of formula (I-G), m is 3, and the R^{1a} groups are at the 4-position, 7-position, and 8-position. In some embodiments of the compound of formula (I-G), m is 3, and the R^{1a} groups are at the 3-position, 5-position, and 6-position. In some embodiments of the compound of formula (I-G), m is 3, and the R^{1a} groups are at the 3-position, 5-position, and 7-position. In some embodiments of the compound of formula (I-G), m is 3, and the R^{1a} groups are at the 3-position, 5-position, and 8-position. In some embodiments of the compound of formula (I-G), m is 3, and the R^{1a} groups are at the 3-position, 6-position, and 7-position. In some embodiments of the compound of formula (I-G), m is 3, and the R^{1a} groups are at the 3-position, 6-position, and 8-position. In some embodiments of the compound of formula (I-G), m is 3, and the R^{1a} groups are at the 3-position, 7-position, and 8-position. In some embodiments of the compound of formula (I-G), m is 3, and the R^{1a} groups are at the 5-position, 6-position, and 7-position. In some embodiments of the compound of formula (I-G), m is 3, and the R^{1a} groups are at the 5-position, 6-position, and 8-position. In some embodiments of the compound of formula (I-G), m is 3, and the R^{1a} groups are at the 5-position, 7-position, and 8-position. In some embodiments of the compound of formula (I-G), m is 3, and the R^{1a} groups are at the 6-position, 7-position, and 8-position. In some embodiments of the compound of formula (I-G), m is 4, and the R^{1a} groups are at the 3-position, 4-position, 5-position, and 6-position. In some embodiments of the compound of formula (I-G), m is 4, and the R^{1a} groups are at the 3-position, 4-position, 5-position, and 7-position. In some embodiments of the compound of formula (I-G), m is 4, and the R^{1a} groups are at the 3-position, 4-position, 5-position, and 8-position. In some embodiments of the compound of formula (I-G), m is 4, and the R^{1a} groups are at the 3-position, 4-position, 6-position, and 7-position. In some embodiments of the compound of formula (I-G), m is 4, and the R^{1a} groups are at the 4-position, 3-position, 6-position, and 8-position. In some embodiments of the compound of formula (I-G), m is 4, and the R^{1a} groups are at the 3-position, 4-position, 7-position, and 8-position. In some embodiments of the compound of formula (I-G), m is 4, and the R^{1a} groups are at the 4-position, 5-position, 6-position, and 7-position. In some embodiments of the compound of formula (I-G), m is 4, and the R^{1a} groups are at the 4-position, 5-position, 6-position, and 8-position. In some embodiments of the compound of formula (I-G), m is 4, and the R^{1a} groups are at the 4-position, 5-position, 7-position, and 8-position. In some embodiments of the compound of formula (I-G), m is 4, and the R^{1a} groups are at the 4-position, 6-position, 7-position, and 8-position. In some embodiments of the compound of formula (I-G), m is 4, and the R^{1a} groups are at the 3-position, 5-position, 6-position, and 7-position. In some embodiments of the compound of formula (I-G), m is 4, and the R^{1a} groups are at the 3-position, 5-position, 6-position, and 8-position. In some embodiments of the compound of formula (I-G), m is 4, and the R^{1a} groups are at the 3-position, 5-position, 7-position, and 8-position. In some embodiments of the compound of formula (I-G), m is 4, and the R^{1a} groups are at the 3-position, 6-position, 7-position, and 8-position.In some embodiments of the compound of formula (I-G), m is 4, and the R^{1a} groups are at the 5-position, 6-position, 7-position, and 8-position. In some embodiments of the compound of formula (I-G), m is 5, and the R^{1a} groups are at the 3-position, 4-position, 5-position, 6-position, and 7-position. In some embodiments of the compound of formula (I-G), m is 5, and the R^{1a} groups are at the 3-position, 4-position, 5-position, 6-position, and 8-position. In some embodiments of the compound of formula (I-G), m is 5, and the R^{1a} groups are at the 3-position, 4-position, 5-position, 7-position, and 8-position. In some embodiments of the compound of formula (I-G), m is 5, and the R^{1a} groups are at the 3-position, 4-position, 6-position, 7-position, and 8-position. In some embodiments of the compound of formula (I-G), m is 5, and the R^{1a} groups are at the 4-position, 5-position, 6-position, 7-position, and 8-position. In some embodiments of the compound of formula (I-G), m is 5, and the R^{1a} groups are at the 3-position, 5-position, 6-position, 7-position, and 8-position. In some embodiments of the compound of formula (I-G), m is 6, and the R^{1a} groups are at the 3-position, 4-position, 5-position, 6-position, 7-position, and 8-position. Whenever more than one R^{1a} group is present, the R^{1a} groups can be chosen independently. In any of these embodiments of the compound of formula (I-G), or a salt thereof, the carbon bearing the CO₂H and NH moieties may be in the “S” configuration or the “R” configuration.

In some embodiments of formula (I-G), including the embodiments that describe the R^{1a} and m variables, each of R¹⁰, R¹¹, R¹² and R¹³ are hydrogen. In some embodiments of formula (I-G), including the embodiments that describe the R^{1a} and m variables, and/or the R¹⁰, R¹¹, R¹² and R¹³ variables, q is 0. In some embodiments of formula (I-G), including the embodiments that describe the R^{1a} and m variables, and/or the R¹⁰, R¹¹, R¹² and R¹³ variables and/or the q variable, p is 3, 4 or 5.

In some embodiments of formula (I-G), R¹⁰, R¹¹, R¹² and R¹³ are hydrogen, p is 3, q is 0 and the compound is of the formula (II-G):

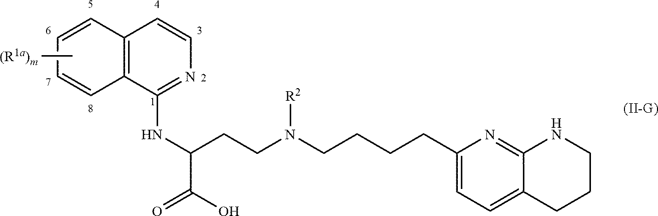

or a salt thereof, wherein R^{1a} and R² are as defined for formula (I), m is 0, 1, 2, 3, 4, 5, or 6 and the positions on the isoquinoline ring are as indicated. All descriptions of R^{1a}, R² and m with reference to formula (I) apply equally to formulae (I-G) and (II-G).

In some embodiments of the compound of formula (I), wherein R¹ is 5- to 10-membered heteroaryl optionally substituted by R^{1a}, the compound is of the formula (I-H):

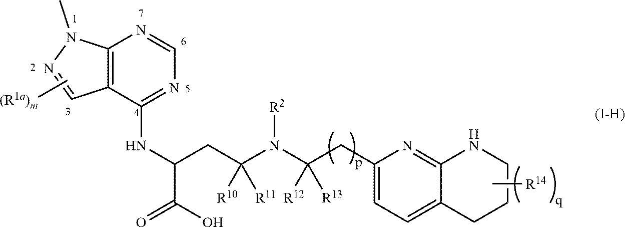

or a salt thereof, wherein R^{1a}, R², R¹⁰, R¹¹, R¹², R¹³, R¹⁴, q and p are as defined for formula (I), m is 0, 1, or 2, and the positions on the 1-methyl-1H-pyrazolo[3,4-d]pyrimidine ring are as indicated.

In one embodiment is provided a compound of the formula (I-H), or a salt thereof, wherein the carbon bearing the CO₂H and NH moieties is in the “S” configuration. In another embodiment is provided a compound of the formula (I-H), or a salt thereof, wherein the carbon bearing the CO₂H and NH moieties is in the “R” configuration. Mixtures of a compound of the formula (I-H) are also embraced, including racemic or non-racemic mixtures of a given compound, and mixtures of two or more compounds of different chemical formulae.

In some embodiments of the compound of formula (I-H), m is 0, 1, or 2, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I-H), m is 0, 1, or 2, and each R^{1a} is, where applicable, independently deuterium, halogen, C₁-C₆ alkyl, C₁-C₆ haloalkyl (which in one variation may be C₁-C₆ perhaloalky), C₁-C₆ alkoxy, hydroxy, —CN, or 5- to 10-membered heteroaryl, wherein the C₁-C₆ alkyl, C₁-C₆ haloalkyl, C₁-C₆ alkoxy, hydroxy, and 5- to 10-membered heteroaryl of R^{1a} are independently optionally substituted by deuterium. In some embodiments of the compound of formula (I-H), m is 1 or 2.

In some embodiments of the compound of formula (I-H), m is 0. In some embodiments of the compound of formula (I-H), m is 1, and R^{1a} is at the 3-position. In some embodiments of the compound of formula (I-H), m is 1, and R^{1a} is at the 6-position. In some embodiments of the compound of formula (I-H), m is 2, and the R^{1a} groups are at the 3-position and 6-position. Whenever more than one R^{1a} group is present, the R^{1a} groups can be chosen independently. In any of these embodiments of the compound of formula (I-H), or a salt thereof, the carbon bearing the CO₂H and NH moieties may be in the “S” configuration or the “R” configuration.

In some embodiments of formula (I-H), including the embodiments that describe the R^{1a} and m variables, each of R¹⁰, R¹¹, R¹² and R¹³ are hydrogen. In some embodiments of formula (I-H), including the embodiments that describe the R^{1a} and m variables, and/or the R¹⁰, R¹¹, R¹² and R¹³ variables, q is 0. In some embodiments of formula (I-H), including the embodiments that describe the R^{1a} and m variables, and/or the R¹⁰, R¹¹, R¹² and R¹³ variables and/or the q variable, p is 3, 4 or 5.

In some embodiments of formula (I-H), R¹⁰, R¹¹, R¹² and R¹³ are hydrogen, p is 3, q is 0 and the compound is of the formula (II-H):

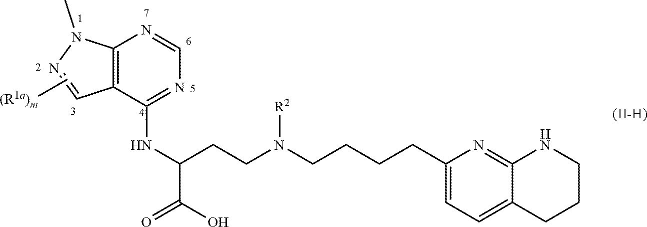

or a salt thereof, wherein R^{1a} and R² are as defined for formula (I), m is 0, 1, or 2, and the positions on the 1-methyl-1H-pyrazolo[3,4-d]pyrimidine ring are as indicated. All descriptions of R^{1a}, R² and m with reference to formula (I) apply equally to formulae (I-H) and (II-H).

Also provided is a compound of formula (I) or (II), or a salt thereof, wherein R¹ is 5-to 10-membered heteroaryl optionally substituted by R^{1a}. In some embodiments, R¹ is unsubstituted 5- to 10-membered heteroaryl (e.g., pyridinyl, pyrimidinyl, quinoxalinyl, quinazolinyl, pyrazolopyrimidinyl, quinolinyl, pyridopyrimidinyl, thienopyrimidinyl, pyridinyl, pyrrolopyrimidinyl, benzothiazolyl, isoquinolinyl, purinyl, or benzooxazolyl). In some embodiments, R¹ is 5- to 10-membered heteroaryl substituted by 1, 2, 3, 4, or 5 R^{1a} groups which may be the same or different, wherein each R^{1a} is independently selected from halogen (e.g., fluoro, chloro, or bromo), C₁-C₆ alkyl optionally substituted by halogen (e.g., —CH₃, —CHF₂, —CF₃, or C(CH₃)₃), C₃-C₆ cycloalkyl (e.g., cyclopropyl), 5- to 10-membered heteroaryl (e.g., pyridinyl or pyrazolyl), C₆-C₁₄ aryl (e.g., phenyl), —CN, -OR³ (e.g., —OCH₃), and -NR⁴R⁵ (e.g., —N(CH₃)₂). In some embodiments, R¹ is 5-membered heteroaryl (e.g., pyrazolyl) substituted by 1, 2, 3, or 4 R^{1a} groups which may be the same or different and is selected from —CH₃, —CH₂F, -CHF₂, and —CF₃. In some embodiments, R¹ is 6-membered heteroaryl (e.g., pyridinyl, pyrimidinyl, or pyrazinyl) substituted by 1, 2, 3, 4, or 5 R^{1a} groups which may be the same or different and is selected from halogen (e.g., fluoro, chloro, or bromo), C₃-C₆ cycloalkyl (e.g., cyclopropyl), 5- to 6-membered heteroaryl (e.g., pyridinyl or pyrazolyl), C₆-C₁₀ aryl (e.g., phenyl), C₁-C₄ alkyl optionally substituted by halogen (e.g., —CH₃, —CF₃ or C(CH₃)₃), —CN, —OR³ (e.g., —OCH₃), and -NR⁴R⁵ (e.g., —N(CH₃)₂). In some embodiments, R¹ is 9-membered heteroaryl (e.g., pyrazolopyrimidinyl, pyrrolopyrimidinyl, thienopyrimidinyl, indazolyl, indolyl, or benzoimidazolyl) substituted by 1, 2, 3, 4, or 5 R^{1a} groups which may be the same or different and is selected from —CH₃, —CH₂F, —CHF₂, and —CF₃. In some embodiments, R¹ is 10-membered heteroaryl (e.g., quinazolinyl) substituted by 1, 2, 3, 4, or 5 R^{1a} groups which may be the same or different and is selected from halogen (e.g., fluoro or chloro), 5- to 6-membered heteroaryl (e.g., pyridinyl), Ci alkyl optionally substituted by halogen (e.g., —CH₃ or —CF₃), and -OR³ (e.g., —OCH₃).

Also provided is a compound of formula (I) or (II), or a salt thereof, wherein R¹ is selected from the group consisting of

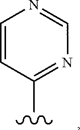

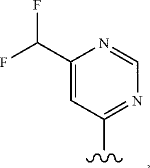

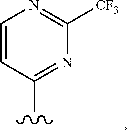

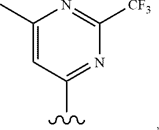

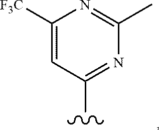

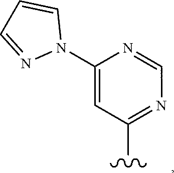

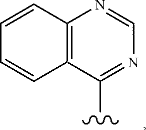

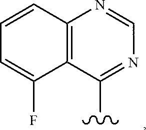

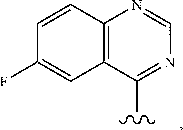

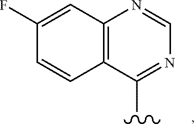

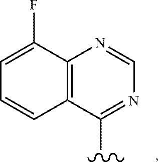

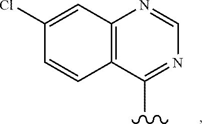

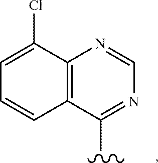

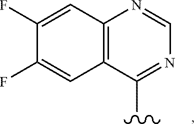

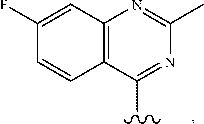

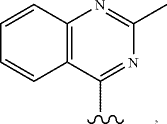

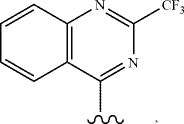

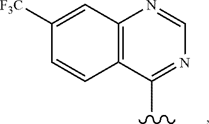

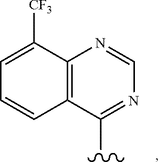

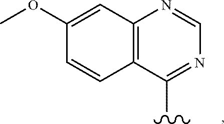

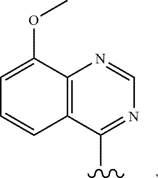

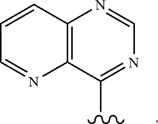

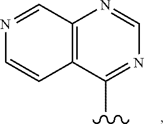

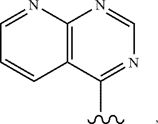

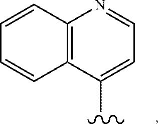

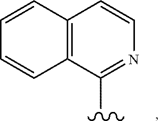

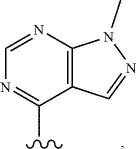

and any of the foregoing groups wherein any one or more hydrogen atom(s) are replaced with deuterium atom(s). Also provided is a compound of formula (I) or (II), or a salt thereof, wherein R¹ is selected from any of the foregoing groups wherein any one or more hydrogen atom(s) are replaced with tritium atom(s). For example, in some embodiments, each hydrogen bonded to a ring carbon in the foregoing groups may be replaced with a corresponding isotope, e.g., deuterium or tritium. Each hydrogen bonded to an acyclic carbon in the foregoing groups, e.g., methyl or methoxy carbons, may be replaced with a corresponding isotope, e.g., deuterium or tritium. Further, for example, the foregoing groups may be perdeuterated, in which every hydrogen is replaced with deuterium, or pertritiated, in which every hydrogen is replaced with tritium. In some embodiments, one or more ring carbons in the foregoing groups may be replaced with ¹³C. For example, in polycyclic rings among the foregoing groups, one or more ring carbons in the ring directly bonded to the rest of the compound may be replaced with ¹³C. In polycyclic rings among the foregoing groups, one or more ring carbons may be replaced with ¹³C in the ring that substitutes or is fused to the ring bonded to the rest of the compound. Further, for example, every ring carbon in the foregoing groups may be replaced with ¹³C.

Also provided is a compound of formula (I) or (II), or a salt thereof, wherein R¹ is selected from the group consisting of

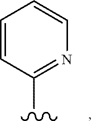

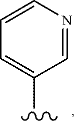

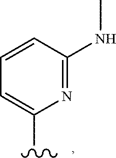

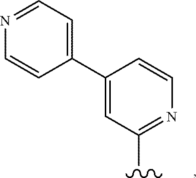

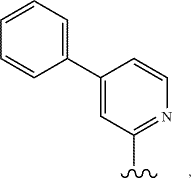

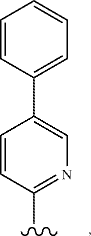

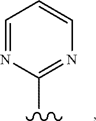

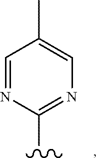

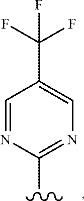

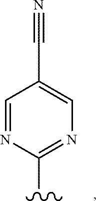

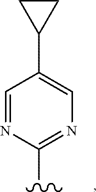

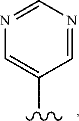

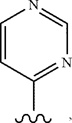

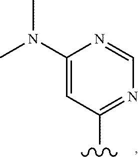

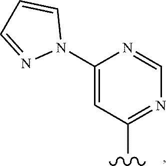

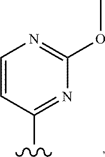

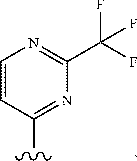

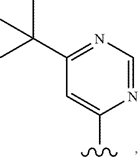

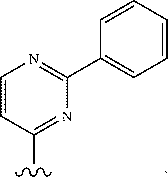

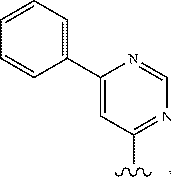

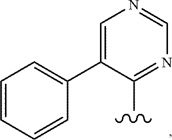

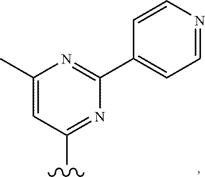

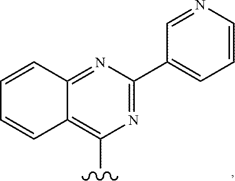

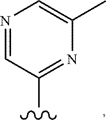

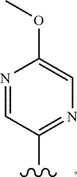

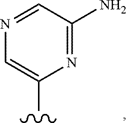

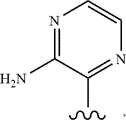

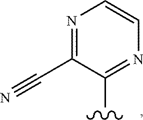

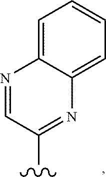

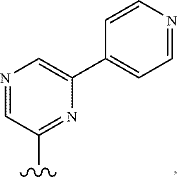

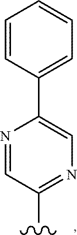

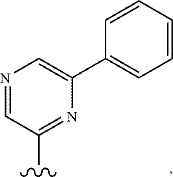

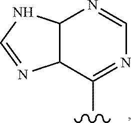

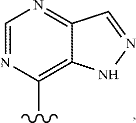

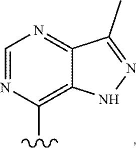

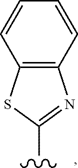

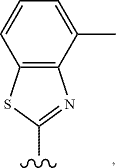

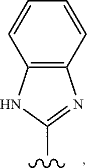

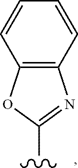

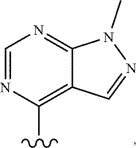

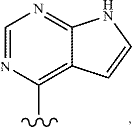

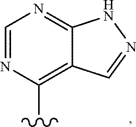

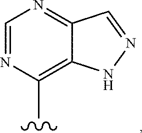

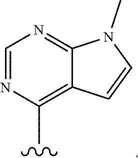

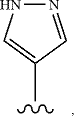

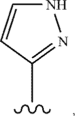

and any of the foregoing groups wherein any one or more hydrogen atom(s) are replaced with deuterium atom(s). Also provided is a compound of formula (I) or (II), or a salt thereof, wherein R¹ is selected from any of the foregoing groups wherein any one or more hydrogen atom(s) are replaced with tritium atom(s). For example, in some embodiments, each hydrogen bonded to a ring carbon in the forgoing groups may be replaced with a corresponding isotope, e.g., deuterium or tritium. Each hydrogen bonded to an acyclic carbon in the forgoing groups, e.g., methyl or methoxy carbons, may be replaced with a corresponding isotope, e.g., deuterium or tritium. Further, for example, the forgoing groups may be perdeuterated, in which every hydrogen is replaced with deuterium, or pertritiated, in which every hydrogen is replaced with tritium. In some embodiments, one or more ring carbons in the forgoing groups may be replaced with ¹³C. For example, in polycyclic rings among the forgoing groups, one or more ring carbons in the ring directly bonded to the rest of the compound may be replaced with ¹³C. In polycyclic rings among the forgoing groups, one or more ring carbons may be replaced with ¹³C in the ring that substitutes or is fused to the ring bonded to the rest of the compound. Further, for example, every ring carbon in the forgoing groups may be replaced with ¹³C.

Also provided is a compound of formula (I) or (II), or a salt thereof, wherein R¹ is selected from the group consisting of

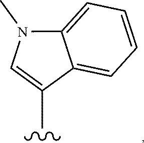

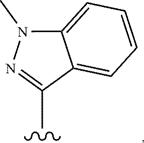

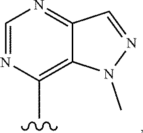

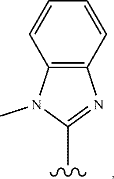

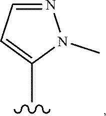

and any of the foregoing groups wherein any one or more hydrogen atom(s) are replaced with deuterium atom(s). Also provided is a compound of formula (I) or (II), or a salt thereof, wherein R¹ is selected from any of the foregoing groups wherein any one or more hydrogen atom(s) are replaced with tritium atom(s). For example, in some embodiments, each hydrogen bonded to a ring carbon in the forgoing groups may be replaced with a corresponding isotope, e.g., deuterium or tritium. Each hydrogen bonded to an acyclic carbon in the forgoing groups, e.g., methyl or methoxy carbons, may be replaced with a corresponding isotope, e.g., deuterium or tritium. Further, for example, the forgoing groups may be perdeuterated, in which every hydrogen is replaced with deuterium, or pertritiated, in which every hydrogen is replaced with tritium. In some embodiments, one or more ring carbons in the forgoing groups may be replaced with ¹³C. For example, in polycyclic rings among the forgoing groups, one or more ring carbons in the ring directly bonded to the rest of the compound may be replaced with ¹³C. In polycyclic rings among the forgoing groups, one or more ring carbons may be replaced with ¹³C in the ring that substitutes or is fused to the ring bonded to the rest of the compound. Further, for example, every ring carbon in the forgoing groups may be replaced with ¹³C.

Also provided is a compound of formula (I) or (II), or a salt thereof, wherein R¹ is selected from the group consisting of

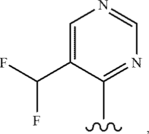

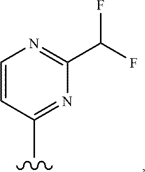

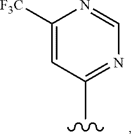

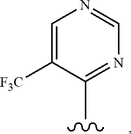

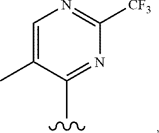

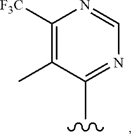

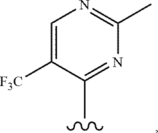

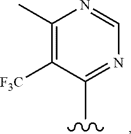

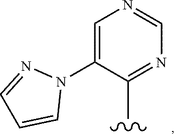

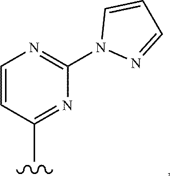

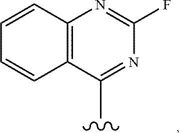

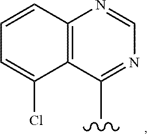

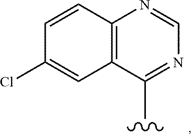

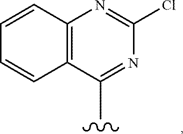

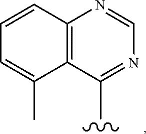

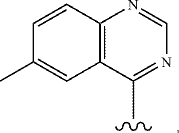

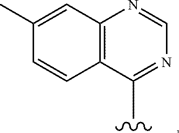

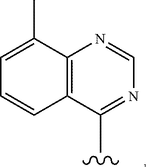

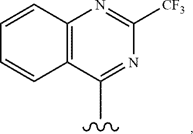

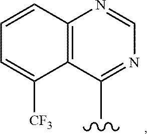

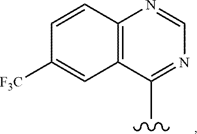

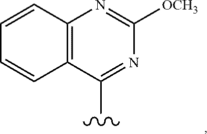

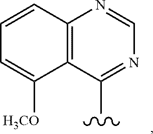

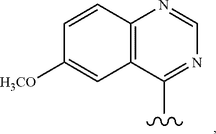

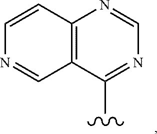

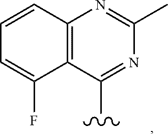

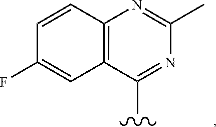

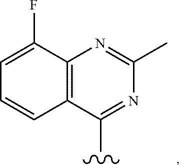

and any of the foregoing groups wherein any one or more hydrogen atom(s) are replaced with deuterium atom(s). Also provided is a compound of formula (I) or (II), or a salt thereof, wherein R¹ is selected from any of the foregoing groups wherein any one or more hydrogen atom(s) are replaced with tritium atom(s). For example, in some embodiments, each hydrogen bonded to a ring carbon in the forgoing groups may be replaced with a corresponding isotope, e.g., deuterium or tritium. Each hydrogen bonded to an acyclic carbon in the forgoing groups, e.g., methyl or methoxy carbons, may be replaced with a corresponding isotope, e.g., deuterium or tritium. Further, for example, the forgoing groups may be perdeuterated, in which every hydrogen is replaced with deuterium, or pertritiated, in which every hydrogen is replaced with tritium. In some embodiments, one or more ring carbons in the forgoing groups may be replaced with ¹³C. For example, in polycyclic rings among the forgoing groups, one or more ring carbons in the ring directly bonded to the rest of the compound may be replaced with ¹³C. In polycyclic rings among the forgoing groups, one or more ring carbons may be replaced with ¹³C in the ring that substitutes or is fused to the ring bonded to the rest of the compound. Further, for example, every ring carbon in the forgoing groups may be replaced with ¹³C.

Also provided is a compound of formula (I) or (II), or a salt thereof, wherein R¹ is selected from the group consisting of

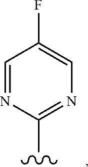

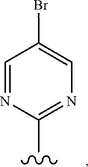

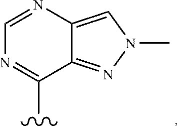

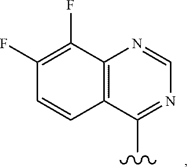

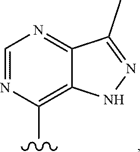

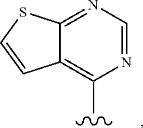

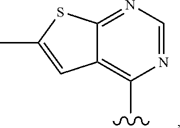

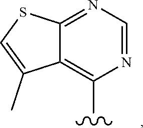

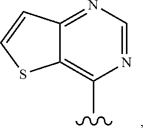

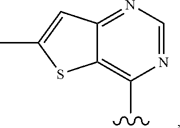

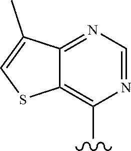

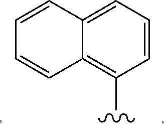

and any of the foregoing groups wherein any one or more hydrogen atom(s) are replaced with deuterium atom(s). Also provided is a compound of formula (I) or (II), or a salt thereof, wherein R¹ is selected from any of the foregoing groups wherein any one or more hydrogen atom(s) are replaced with tritium atom(s). For example, in some embodiments, each hydrogen bonded to a ring carbon in the forgoing groups may be replaced with a corresponding isotope, e.g., deuterium or tritium. Each hydrogen bonded to an acyclic carbon in the forgoing groups, e.g., methyl or methoxy carbons, may be replaced with a corresponding isotope, e.g., deuterium or tritium. Further, for example, the forgoing groups may be perdeuterated, in which every hydrogen is replaced with deuterium, or pertritiated, in which every hydrogen is replaced with tritium. In some embodiments, one or more ring carbons in the forgoing groups may be replaced with ¹³C. For example, in polycyclic rings among the forgoing groups, one or more ring carbons in the ring directly bonded to the rest of the compound may be replaced with ¹³C. In polycyclic rings among the forgoing groups, one or more ring carbons may be replaced with ¹³C in the ring that substitutes or is fused to the ring bonded to the rest of the compound. Further, for example, every ring carbon in the forgoing groups may be replaced with ¹³C.

The R¹ groups described herein as moieties (shown with a [symbol] symbol) are shown as attached at specific positions (e.g., pyrimid-4-yl, quinazolin-4-yl, isoquinolin-1-yl) but they can also be attached via any other available valence (e.g., pyrimid-2-yl). In some embodiments of the compound of formula (I) or (II), or a salt thereof, R¹ is

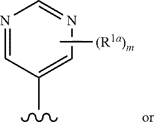

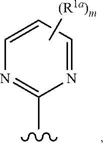

wherein m is 0, 1, 2, or 3 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I) or (II), or a salt thereof, R¹ is

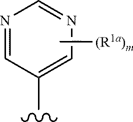

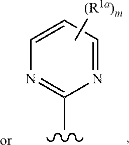

wherein m is 1, 2, or 3 and each R^{1a} is independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In another embodiment, R¹ is

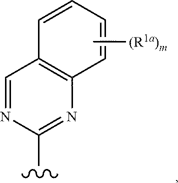

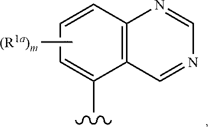

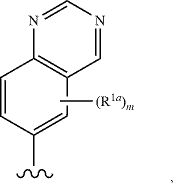

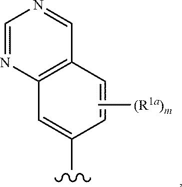

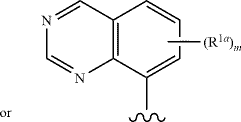

wherein m is 0, 1, 2, 3, 4, or 5 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further embodiment of the compound of formula (I) or (II), or a salt thereof, R¹ is

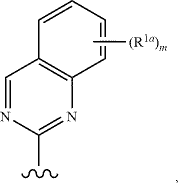

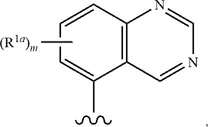

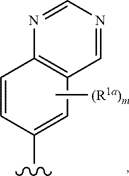

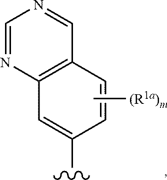

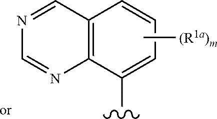

wherein m is 1, 2, 3, 4, or 5 andeach R^{1a} is independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium. In a further variation of such embodiments, each R^{1a} is, where applicable, independently deuterium, halogen, C₁-C₆ alkyl, C₁-C₆ haloalkyl (which in one variation may be C₁-C₆ perhaloalky), C₁-C₆ alkoxy, hydroxy, —CN, or 5- to 10-membered heteroaryl, wherein the C₁-C₆ alkyl, C₁-C₆ haloalkyl, C₁-C₆ alkoxy, hydroxy, and 5- to 10-membered heteroaryl of R^{1a} are independently optionally substituted by deuterium.

In some embodiments of the compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, R² is C₁-C₆ alkyl optionally substituted by R^{2a}. In some embodiments, R² is C₁-C₆ alkyl optionally substituted by R^{2a} where R^{2a} is: halogen (e.g., fluoro); C₃-C₈ cycloalkyl optionally substituted by halogen (e.g., cyclobutyl optionally substituted by fluoro); 5- to 10-membered heteroaryl optionally substituted by C₁-C₆ alkyl (e.g., pyrazolyl optionally substituted by methyl); -S(O)₂R³; -NR⁴R⁵; -NR³C(O)R⁴; oxo; or -OR³. In some embodiments, R² is C₁-C₆ alkyl optionally substituted by R^{2a} where R^{2a} is: halogen (e.g., fluoro); C₃-C₈ cycloalkyl optionally substituted by halogen (e.g., cyclobutyl optionally substituted by fluoro); 5- to 10-membered heteroaryl optionally substituted by C₁-C₆ alkyl (e.g., pyrazolyl optionally substituted by methyl); 3- to 12-membered heterocyclyl optionally substituted by halogen (e.g., oxetanyl optionally substituted by fluoro), -S(O)₂R³; -NR⁴R⁵; -NR³C(O)R⁴; oxo; or -OR³. In some embodiments, R² is C₁-C₆ alkyl optionally substituted by -OR³ wherein R³ is: hydrogen; C₁-C₆ alkyl optionally substituted by halogen (e.g., methyl, ethyl, difluoromethyl, —CH₂CHF₂, and —CH₂CF₃); C₃-C₆ cycloalkyl optionally substituted by halogen (e.g., cyclopropyl substituted by fluoro); C₆-C₁₄ aryl optionally substituted by halogen (e.g., phenyl optionally substituted by fluoro); or 5- to 6-membered heteroaryl optionally substituted by halogen or C₁-C₆ alkyl (e.g., pyridinyl optionally substituted by fluoro or methyl). In some embodiments, R² is —CH₂CH₂OCH₃. In some embodiments, R² is C₁-C₆ alkyl substituted by both halogen and OR³. In some embodiments, R² is n-propyl substituted by both halogen and alkoxy (e.g., —CH₂CH(F)CH₂OCH₃). In some embodiments where R² is indicated as optionally substituted by R^{2a}, the R² moiety is unsubstituted. In some embodiments where R² is indicated as optionally substituted by R^{2a}, the R² moiety is substituted by one R^{2a}. In some embodiments where R² is indicated as optionally substituted by R^{2a}, the R² moiety is substituted by 2 to 6 or 2 to 5 or 2 to 4 or 2 to 3 R^{2a} moieties, which may be the same or different.

In some embodiments of the compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, R² is C₁-C₆ alkyl optionally substituted by R^{2a}. In some embodiments, R² is C₁-C₆ alkyl optionally substituted by R^{2a} where R^{2a} is: halogen (e.g., fluoro); C₃-C₈ cycloalkyl optionally substituted by halogen (e.g., cyclobutyl optionally substituted by fluoro); 5- to 10-membered heteroaryl optionally substituted by C₁-C₆ alkyl (e.g., pyrazolyl optionally substituted by methyl); -S(O)₂R³; -NR⁴R⁵; -NR³C(O)R⁴; oxo; or -OR³. In some embodiments, R² is C₁-C₆ alkyl optionally substituted by R^{2a} where R^{2a} is: halogen (e.g., fluoro); C₃-C₈ cycloalkyl optionally substituted by halogen (e.g., cyclobutyl optionally substituted by fluoro); 5- to 10-membered heteroaryl optionally substituted by C₁-C₆ alkyl (e.g., pyrazolyl optionally substituted by methyl); 3- to 12-membered heterocyclyl optionally substituted by halogen (e.g., oxetanyl optionally substituted by fluoro); -S(O)₂R³; -NR⁴R⁵; -NR³C(O)R⁴; oxo; or -OR³. In some embodiments, R² is C₁-C₆ alkyl optionally substituted by R^{2a} where R^{2a} is: halogen (e.g., fluoro); C₃-C₈ cycloalkyl optionally substituted by halogen (e.g., cyclobutyl optionally substituted by fluoro); C₆-C₁₄ aryl (e.g., phenyl); 5- to 10-membered heteroaryl optionally substituted by C₁-C₆ alkyl (e.g., thiazolyl or pyrazolyl optionally substituted by methyl); 3- to 12-membered heterocyclyl optionally substituted by halogen or oxo (e.g., R^{2a} is: oxetanyl optionally substituted by fluoro; tetrahydrofuranyl; pyrrolidinyl optionally substituted by oxo; morpholinyl optionally substituted by oxo; or dioxanyl); -S(O)₂R³; -NR⁴R⁵; -NR³C(O)R⁴; oxo; -OR³; or —CN. In some embodiments, R² is C₁-C₆ alkyl optionally substituted by -OR³ wherein R³ is: hydrogen; C₁-C₆ alkyl optionally substituted by halogen (e.g., methyl, ethyl, difluoromethyl, -CH₂CHF₂, and —CH₂CF₃); C₃-C₆ cycloalkyl optionally substituted by halogen (e.g., cyclopropyl substituted by fluoro); C₆-C₁₄ aryl optionally substituted by halogen (e.g., phenyl optionally substituted by fluoro); or 5- to 6-membered heteroaryl optionally substituted by halogen or C₁-C₆ alkyl (e.g., pyridinyl optionally substituted by fluoro or methyl). In some embodiments, R² is —CH₂CH₂OCH₃. In some embodiments, R² is C₁-C₆ alkyl substituted by both halogen and OR³. In some embodiments, R² is n-propyl substituted by both halogen and alkoxy (e.g., —CH₂CH(F)CH₂OCH₃). In some embodiments where R² is indicated as optionally substituted by R^{2a}, the R² moiety is unsubstituted. In some embodiments where R² is indicated as optionally substituted by R^{2a}, the R² moiety is substituted by one R^{2a}. In some embodiments where R² is indicated as optionally substituted by R^{2a}, the R² moiety is substituted by 2 to 6 or 2 to 5 or 2 to 4 or 2 to 3 R^{2a} moieties, which may be the same or different. In some embodiments, R² is C₁-C₆ alkyl substituted by two halogen groups, which may be the same or different (e.g., two fluoro groups). In some embodiments, R² is C₁-C₆ alkyl substituted by two —OR³ groups, which may be the same or different (e.g., two —OH groups, one —OH group and one —OCH₃ group, or two —OCH₃ groups). In some embodiments, R² is C₁-C₆ alkyl substituted by one halogen group (e.g., fluoro) and one -OR³ group (e.g., —OH or —OCH₃). In some embodiments, R² is C₁-C₆ alkyl substituted by two halogen groups, which may be the same or different (e.g., two fluoro groups), and one -OR³ group (e.g., —OH or -OCH₃). In some embodiments, R² is C₁-C₆ alkyl substituted by one halogen group (e.g., fluoro) and two -OR³ groups, which may be the same or different (e.g., two —OH groups, one —OH group and one -OCH₃ group, or two —OCH₃ groups).

In some embodiments of the compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, R² is C₃-C₆ cycloalkyl optionally substituted by R^{2b}. In some embodiments, R² is C₃-C₆ cycloalkyl substituted by 1 or 2 R^{2b} moieties which may be the same or different. In some embodiments, R² is C₃-C₄ cycloalkyl optionally substituted by halogen (e.g., unsubstituted cyclopropyl or cyclobutyl optionally substituted by fluoro). In some embodiments, R² is C₃-C₄ cycloalkyl optionally substituted by deuterium, or tritium atom(s). For example, in some embodiments, each hydrogen bonded to a ring carbon in the forgoing groups may be replaced with a corresponding isotope, e.g., deuterium or tritium. Each hydrogen bonded to an acyclic carbon in the forgoing groups, e.g., methyl or methoxy carbons, may be replaced with a corresponding isotope, e.g., deuterium or tritium. Further, for example, the forgoing groups may be perdeuterated, in which every hydrogen is replaced with deuterium, or pertritiated, in which every hydrogen is replaced with tritium. In some embodiments, one or more ring carbons in the forgoing groups may be replaced with ¹³C. For example, in polycyclic rings among the forgoing groups, one or more ring carbons in the ring directly bonded to the rest of the compound may be replaced with ¹³C. In polycyclic rings among the forgoing groups, one or more ring carbons may be replaced with ¹³C in the ring that substitutes or is fused to the ring bonded to the rest of the compound. Further, for example, every ring carbon in the forgoing groups may be replaced with ¹³C.

In some embodiments of the compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, R² is hydrogen.

In some embodiments of the compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, R² is —O—C₁—C₆ alkyl optionally substituted by R^{2a}. In some embodiments, R² is -OCH₃.

Also provided is a compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, wherein R² is selected from the group consisting of

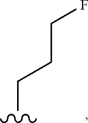

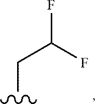

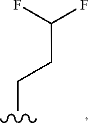

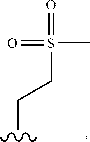

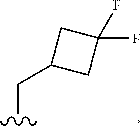

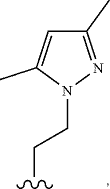

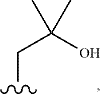

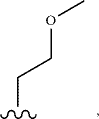

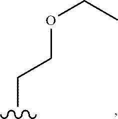

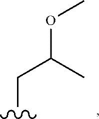

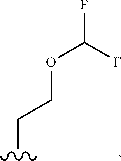

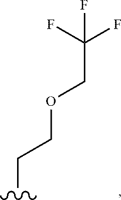

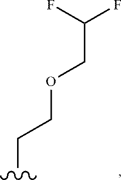

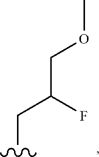

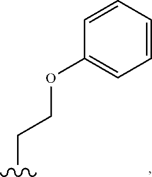

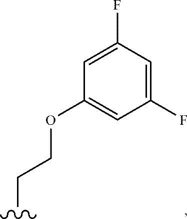

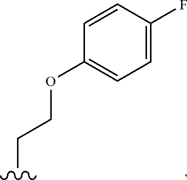

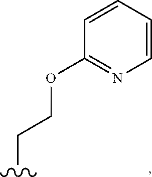

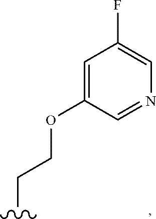

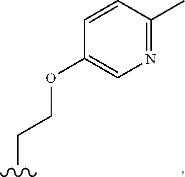

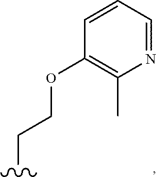

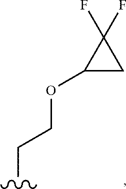

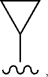

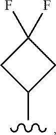

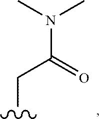

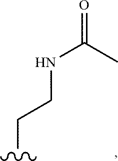

and any of the foregoing groups wherein any one or more hydrogen atom(s) are replaced with deuterium atom(s).

Also provided is a compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, wherein R² is selected from the group consisting of

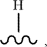

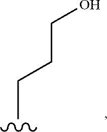

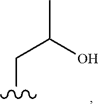

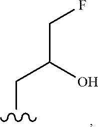

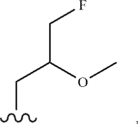

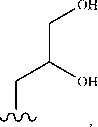

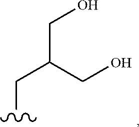

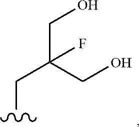

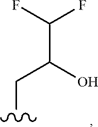

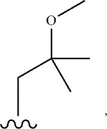

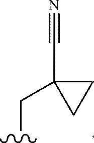

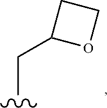

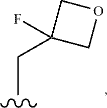

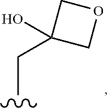

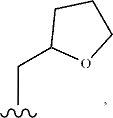

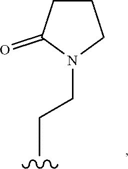

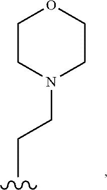

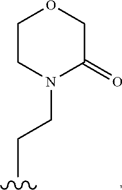

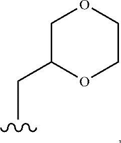

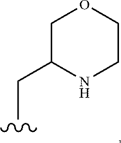

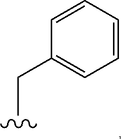

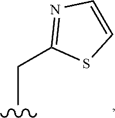

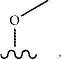

and any of the foregoing groups wherein any one or more hydrogen atom(s) are replaced with deuterium atom(s).

Also provided is a compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, wherein R² is

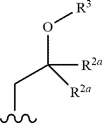

wherein R³ and each R^{2a} are as defined for formula (I).

Also provided is a compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, wherein R² is

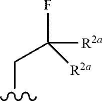

wherein each R^{2a} are as defined for formula (I).

Also provided is a compound of formula (I), (II), (I-A), (II-A), (I-B), (II-B), (I-C), (II-C), (I-D), (II-D), (I-E), (II-E), (I-F), (II-F), (I-G), (II-G), (I-H) or (II-H), or a salt thereof, wherein R² is

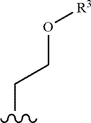

wherein R³ is as defined for formula (I).

In one embodiment of formula (I), the tetrahydronaphthyridine group is disubstituted with deuterium at the 2-position.

In one aspect, provided is a compound of formula (I), or a salt thereof (including a pharmaceutically acceptable salt thereof), wherein the compound or salt thereof has any one or more of the following structural features (“SF”):
(SFI) p is 3;
(SFII) each R¹⁰, R¹¹, R¹², R¹³ is hydrogen;
(SFIII) R¹ is:
   (A) unsubstituted 5- to 10-membered heteroaryl;
   (B) 5- to 10-membered heteroaryl substituted by 1, 2, 3, 4 or 5 R^{1a} groups which may be the same or different;
   wherein the 5- to 10-membered heteroaryl of (III)(A) and (III)(B) is:
      (i) pyridinyl;
      (ii) pyrimidinyl;
      (iii) quinoxalinyl;
      (iv) quinazolinyl;
      (v) pyrazolopyrimidinyl;
      (vi) quinolinyl;
      (vii) pyridopyrimidinyl;
      (viii) thienopyrimidinyl;
      (ix) purinyl;
      (x) pyrrolopyrimidinyl;
      (xi) benzooxazolyl;
      (xii) benzothiazolyl;
      (xiii) isoquinolinyl;
      (xiv) indolyl;
      (xv) benzoimidazolyl;
      (xvi) pyrazinyl;
      (xvii) indazolyl; or
      (xviii) pyrazolyl;
   (C) unsubstituted naphthalenyl; or
   (D) naphthalenyl substituted by 1, 2, 3, 4 or 5 R^{1a} groups which may be the same or different;
(SFIV) each R^{1a} is:
   (A) halogen, such as fluoro, chloro, or bromo;
   (B) C₁-C₆ alkyl optionally substituted by halogen, such as —CH₃, —CHF₂, —CF₃, or C(CH₃)₃;
   (C) C₃-C₆ cycloalkyl, such as cyclopropyl;
   (D) 5- to 10-membered heteroaryl, such as pyridinyl or pyrazolyl;
   (E) C₆-C₁₄ aryl, such as phenyl;
   (F) —CN;
   (G) -OR³, such as —OCH₃; or
   (H) -NR⁴R⁵, such as —N(CH₃)₂;
(SFV) R² is:
   (A) unsubstituted C₁-C₆ alkyl, such as C₁-C₂ alkyl;
   (B) C₁-C₆ alkyl, such as C₁-C₂ alkyl, each of which is substituted by 1, 2, 3, 4 or 5 R^{2a} groups which may be the same or different;
   (C) unsubstituted —O—C₁—C₆ alkyl, such as —O—C₁—C₂ alkyl;
   (D) —O—C₁—C₆ alkyl, such as —O—C₁—C₂ alkyl, each of which is substituted by 1, 2, 3, 4 or 5 R^{2a} groups which may be the same or different;
   (E) unsubstituted C₃-C₆ cycloalkyl, such as cyclopropyl or cyclobutyl; or
   (F) C₃-C₆ cycloalkyl, such as cyclopropyl or cyclobutyl, each of which is substituted by 1, 2, 3, 4 or 5 R^{2b} groups which may be the same or different; and
(SFVI) R^{2a} is:
   (A) halogen, such as fluoro;
   (B) C₃-C₈ cycloalkyl, such as cyclopropyl or cyclobutyl, each of which is optionally substituted by halogen;
   (C) 5- to 10-membered heteroaryl optionally substituted by C₁-C₆ alkyl, such as pyrazolyl substituted by methyl;
   (D) 3- to 12-membered heterocyclyl optionally substituted by halogen or oxo, such as oxetanyl optionally substituted by fluoro, unsubstituted tetrahydrofuranyl, pyrrolidinyl substituted by oxo, unsubstituted morpholinyl, morpholinyl substituted by oxo, or dioxanyl;
   (E) -S(O)₂R³, such as —S(O)₂CH₃;
   (F) -C(O)NR⁴R⁵, such as —C(O)N(CH₃)₂;
   (G) -NR³C(O)R⁴, such as —NHC(O)CH₃; or
   (H) -OR³, wherein R³ is:
      (i) hydrogen;
      (ii) —CH₃;
      (iii) —CH₂CH₃;
      (iv) —CH₂CHF₂;
      (v) —CH₂CF₃;
      (vi) phenyl substituted by 0-2 fluoro groups; or
      (vii) pyridinyl substituted by 0-1 methyl group.

It is understood that compounds of formula (I) or any variation thereof described herein, or a salt thereof, can in one embodiment have any one or more of the structural features as noted above. For example, compounds of formula (I) or any variation thereof described herein, or a salt thereof, can in one embodiment have the following structural features: one or two or three or all of (SFI), (SFII), (SFIII) and (SFV). In one such example, a compound of formula (I) or any variation thereof described herein, or a salt thereof, can in one embodiment have the following structural features: (SFI) and any one or two or all of (SFII), (SFIII) and (SFV) or any sub-embodiment thereof. In one such example, a compound of formula (I) or any variation thereof described herein, or a salt thereof, can in one embodiment have the following structural features: (SFII) and any one or two or all of (SFI), (SFIII) and (SFV) or any sub-embodiment thereof. In one such example, a compound of formula (I) or any variation thereof described herein, or a salt thereof, can in one embodiment have the following structural features: (SFIII) and any one or two or all of (SFI), (SFII) and (SFV) or any sub-embodiment thereof. In one such example, a compound of formula (I) or any variation thereof described herein, or a salt thereof, can in one embodiment have the following structural features: (SFV) and any one or two or all of (SFI), (SFII) and (SFIII) or any sub-embodiment thereof. It is understood that the sub-embodiments of structural features can likewise be combined in any manner. Although specific combinations of structural features are specifically noted below, it is understood that each and every combination of features is embraced. In one aspect of this variation, (SFI) and (SFII) apply. In another variation, (SFI) and (SFIII) apply. In another variation, (SFI) and (SFV) apply. In another variation, (SFII) and (SFIII) apply. In another variation, (SFII) and (SFV) apply. In another variation, (SFIII) and (SFV) apply. In another variation, (SFI), (SFII), and (SFIII) apply. In another variation, (SFI), (SFII), and (SFV) apply. In another variation, (SFI), (SFIII), and (SFV) apply. In another variation, (SFII), (SFIII), and (SFV) apply. It is understood that each sub-embodiment of the structural features apply. For example, (SFIII) is (SFIII)(A)(i), (SFIII)(A)(ii),(SFIII)(A)(iii), (SFIII)(A)(iv), (SFIII)(A)(v), (SFIII)(A)(vi), (SFIII)(A)(vii), (SFIII)(A)(viii), (SFIII)(A)(ix), (SFIII)(A)(x), (SFIII)(A)(xi), (SFIII)(A)(xii), (SFIII)(A)(xiii), (SFIII)(A)(xiv), (SFIII)(A)(xv), (SFIII)(A)(xvi), (SFIII)(A)(xvii), (SFIII)(A)(xviii), (SFIII)(B)(i), (SFIII)(B)(ii), (SFIII)(B)(iii), (SFIII)(B)(iv), (SFIII)(B)(v), (SFIII)(B)(vi), (SFIII)(B)(vii), (SFIII)(B)(viii), (SFIII)(B)(ix), (SFIII)(B)(x), (SFIII)(B)(xi), (SFIII)(B)(xii), (SFIII)(B)(xiii), (SFIII)(B)(xiv), (SFIII)(B)(xv), (SFIII)(B)(xvi), (SFIII)(B)(xvii), (SFIII)(B)(xviii), (SFIII)(C), or (SFIII)(D). In one aspect of this variation, (SFV) is (SFV)(A), (SFV)(B), (SFV)(C), (SFV)(D), (SFV)(E), or (SFV)(F).

In another variation, (SFI), (SFII), (SFIII)(A)(i), (SFV)(B), and (SFVI)(A) apply. In another variation, (SFI), (SFII), (SFIII)(A)(ii), (SFV)(B), and (SFVI)(A) apply. In another variation, (SFI), (SFII), (SFIII)(A)(iii), (SFV)(B), and (SFVI)(A) apply. In another variation, (SFI), (SFII), (SFIII)(A)(iv), (SFV)(B), and (SFVI)(A) apply. In another variation, (SFI), (SFII), (SFIII)(A)(v), (SFV)(B), and (SFVI)(A) apply. In another variation, (SFI), (SFII), (SFIII)(A)(vi), (SFV)(B), and (SFVI)(A) apply. In another variation, (SFI), (SFII), (SFIII)(A)(vii), (SFV)(B), and (SFVI)(A) apply. In another variation, (SFI), (SFII), (SFIII)(A)(viii), (SFV)(B), and (SFVI)(A) apply. In another variation, (SFI), (SFII), (SFIII)(A)(ix), (SFV)(B), and (SFVI)(A) apply. In another variation, (SFI), (SFII), (SFIII)(A)(x), (SFV)(B), and (SFVI)(A) apply. In another variation, (SFI), (SFII), (SFIII)(A)(xi), (SFV)(B), and (SFVI)(A) apply. In another variation, (SFI), (SFII), (SFIII)(A)(xii), (SFV)(B), and (SFVI)(A) apply. In another variation, (SFI), (SFII), (SFIII)(A)(xiii), (SFV)(B), and (SFVI)(A) apply. In another variation, (SFI), (SFII), (SFIII)(B)(ii), (SFIV)(A), (SFV)(B), and (SFVI)(A) apply. In another variation, (SFI), (SFII), (SFIII)(B)(ii), (SFIV)(B), (SFV)(B), and (SFVI)(A) apply. In another variation, (SFI), (SFII), (SFIII)(B)(ii), (SFIV)(C), (SFV)(B), and (SFVI)(A) apply. In another variation, (SFI), (SFII), (SFIII)(B)(ii), (SFIV)(D), (SFV)(B), and (SFVI)(A) apply. In another variation, (SFI), (SFII), (SFIII)(B)(ii), (SFIV)(E), (SFV)(B), and (SFVI)(A) apply. In another variation, (SFI), (SFII), (SFIII)(B)(ii), (SFIV)(F), (SFV)(B), and (SFVI)(A) apply. In another variation, (SFI), (SFII), (SFIII)(B)(ii), (SFIV)(G), (SFV)(B), and (SFVI)(A) apply. In another variation, (SFI), (SFII), (SFIII)(B)(ii), (SFIV)(H), (SFV)(B), and (SFVI)(A) apply. In another variation, (SFI), (SFII), (SFIII)(B)(iv), (SFIV)(A), (SFV)(B), and (SFVI)(A) apply. In another variation, (SFI), (SFII), (SFIII)(B)(iv), (SFIV)(B), (SFV)(B), and (SFVI)(A) apply. In another variation, (SFI), (SFII), (SFIII)(B)(iv), (SFIV)(C), (SFV)(B), and (SFVI)(A) apply. In another variation, (SFI), (SFII), (SFIII)(B)(iv), (SFIV)(D), (SFV)(B), and (SFVI)(A) apply. In another variation, (SFI), (SFII), (SFIII)(B)(iv), (SFIV)(E), (SFV)(B), and (SFVI)(A) apply. In another variation, (SFI), (SFII), (SFIII)(B)(iv), (SFIV)(F), (SFV)(B), and (SFVI)(A) apply. In another variation, (SFI), (SFII), (SFIII)(B)(iv), (SFIV)(G), (SFV)(B), and (SFVI)(A) apply. In another variation, (SFI), (SFII), (SFIII)(B)(iv), (SFIV)(H), (SFV)(B), and (SFVI)(A) apply. In another variation, (SFI), (SFII), (SFIII)(B)(vii), (SFIV)(A), (SFV)(B), and (SFVI)(A) apply. In another variation, (SFI), (SFII), (SFIII)(B)(vii), (SFIV)(B), (SFV)(B), and (SFVI)(A) apply. In another variation, (SFI), (SFII), (SFIII)(B)(vii), (SFIV)(C), (SFV)(B), and (SFVI)(A) apply. In another variation, (SFI), (SFII), (SFIII)(B)(vii), (SFIV)(D), (SFV)(B), and (SFVI)(A) apply. In another variation, (SFI), (SFII), (SFIII)(B)(vii), (SFIV)(E), (SFV)(B), and (SFVI)(A) apply. In another variation, (SFI), (SFII), (SFIII)(B)(vii), (SFIV)(F), (SFV)(B), and (SFVI)(A) apply. In another variation, (SFI), (SFII), (SFIII)(B)(vii), (SFIV)(G), (SFV)(B), and (SFVI)(A) apply. In another variation, (SFI), (SFII), (SFIII)(B)(vii), (SFIV)(H), (SFV)(B), and (SFVI)(A) apply. In another variation, (SFI), (SFII), (SFIII)(B)(xvi), (SFIV)(A), (SFV)(B), and (SFVI)(A) apply. In another variation, (SFI), (SFII), (SFIII)(B)(xvi), (SFIV)(B), (SFV)(B), and (SFVI)(A) apply. In another variation, (SFI), (SFII), (SFIII)(B)(xvi), (SFIV)(C), (SFV)(B), and (SFVI)(A) apply. In another variation, (SFI), (SFII), (SFIII)(B)(xvi), (SFIV)(D), (SFV)(B), and (SFVI)(A) apply. In another variation, (SFI), (SFII), (SFIII)(B)(xvi), (SFIV)(E), (SFV)(B), and (SFVI)(A) apply. In another variation, (SFI), (SFII), (SFIII)(B)(xvi), (SFIV)(F), (SFV)(B), and (SFVI)(A) apply. In another variation, (SFI), (SFII), (SFIII)(B)(xvi), (SFIV)(G), (SFV)(B), and (SFVI)(A) apply. In another variation, (SFI), (SFII), (SFIII)(B)(xvi), (SFIV)(H), (SFV)(B), and (SFVI)(A) apply. In another variation, (SFI), (SFII), (SFIII)(B)(v), (SFIV)(B), (SFV)(B), and (SFVI)(A) apply. In another variation, (SFI), (SFII), (SFIII)(B)(viii), (SFIV)(B), (SFV)(B), and (SFVI)(A) apply. In another variation, (SFI), (SFII), (SFIII)(B)(x), (SFIV)(B), (SFV)(B), and (SFVI)(A) apply. In another variation, (SFI), (SFII), (SFIII)(B)(xii), (SFIV)(B), (SFV)(B), and (SFVI)(A) apply. In another variation, (SFI), (SFII), (SFIII)(B)(xiv), (SFIV)(B), (SFV)(B), and (SFVI)(A) apply. In another variation, (SFI), (SFII), (SFIII)(B)(xv), (SFIV)(B), (SFV)(B), and (SFVI)(A) apply. In another variation, (SFI), (SFII), (SFIII)(B)(xvii), (SFIV)(B), (SFV)(B), and (SFVI)(A) apply. In another variation, (SFI), (SFII), (SFIII)(B)(xviii), (SFIV)(B), (SFV)(B), and (SFVI)(A) apply.

In another variation, (SFI), (SFII), (SFIII)(A)(i), (SFV)(B), and (SFVI)(H)(ii) apply. In another variation, (SFI), (SFII), (SFIII)(A)(ii), (SFV)(B), and (SFVI)(H)(ii) apply. In another variation, (SFI), (SFII), (SFIII)(A)(iii), (SFV)(B), and (SFVI)(H)(ii) apply. In another variation, (SFI), (SFII), (SFIII)(A)(iv), (SFV)(B), and (SFVI)(H)(ii) apply. In another variation, (SFI), (SFII), (SFIII)(A)(v), (SFV)(B), and (SFVI)(H)(ii) apply. In another variation, (SFI), (SFII), (SFIII)(A)(vi), (SFV)(B), and (SFVI)(H)(ii) apply. In another variation, (SFI), (SFII), (SFIII)(A)(vii), (SFV)(B), and (SFVI)(H)(ii) apply. In another variation, (SFI), (SFII), (SFIII)(A)(viii), (SFV)(B), and (SFVI)(H)(ii) apply. In another variation, (SFI), (SFII), (SFIII)(A)(ix), (SFV)(B), and (SFVI)(H)(ii) apply. In another variation, (SFI), (SFII), (SFIII)(A)(x), (SFV)(B), and (SFVI)(H)(ii) apply. In another variation, (SFI), (SFII), (SFIII)(A)(xi), (SFV)(B), and (SFVI)(H)(ii) apply. In another variation, (SFI), (SFII), (SFIII)(A)(xii), (SFV)(B), and (SFVI)(H)(ii) apply. In another variation, (SFI), (SFII), (SFIII)(A)(xiii), (SFV)(B), and (SFVI)(H)(ii) apply. In another variation, (SFI), (SFII), (SFIII)(B)(ii), (SFIV)(A), (SFV)(B), and (SFVI)(H)(ii) apply. In another variation, (SFI), (SFII), (SFIII)(B)(ii), (SFIV)(B), (SFV)(B), and (SFVI)(H)(ii) apply. In another variation, (SFI), (SFII), (SFIII)(B)(ii), (SFIV)(C), (SFV)(B), and (SFVI)(H)(ii) apply. In another variation, (SFI), (SFII), (SFIII)(B)(ii), (SFIV)(D), (SFV)(B), and (SFVI)(H)(ii) apply. In another variation, (SFI), (SFII), (SFIII)(B)(ii), (SFIV)(E), (SFV)(B), and (SFVI)(H)(ii) apply. In another variation, (SFI), (SFII), (SFIII)(B)(ii), (SFIV)(F), (SFV)(B), and (SFVI)(H)(ii) apply. In another variation, (SFI), (SFII), (SFIII)(B)(ii), (SFIV)(G), (SFV)(B), and (SFVI)(H)(ii) apply. In another variation, (SFI), (SFII), (SFIII)(B)(ii), (SFIV)(H), (SFV)(B), and (SFVI)(H)(ii) apply. In another variation, (SFI), (SFII), (SFIII)(B)(iv), (SFIV)(A), (SFV)(B), and (SFVI)(H)(ii) apply. In another variation, (SFI), (SFII), (SFIII)(B)(iv), (SFIV)(B), (SFV)(B), and (SFVI)(H)(ii) apply. In another variation, (SFI), (SFII), (SFIII)(B)(iv), (SFIV)(C), (SFV)(B), and (SFVI)(H)(ii) apply. In another variation, (SFI), (SFII), (SFIII)(B)(iv), (SFIV)(D), (SFV)(B), and (SFVI)(H)(ii) apply. In another variation, (SFI), (SFII), (SFIII)(B)(iv), (SFIV)(E), (SFV)(B), and (SFVI)(H)(ii) apply. In another variation, (SFI), (SFII), (SFIII)(B)(iv), (SFIV)(F), (SFV)(B), and (SFVI)(H)(ii) apply. In another variation, (SFI), (SFII), (SFIII)(B)(iv), (SFIV)(G), (SFV)(B), and (SFVI)(H)(ii) apply. In another variation, (SFI), (SFII), (SFIII)(B)(iv), (SFIV)(H), (SFV)(B), and (SFVI)(H)(ii) apply. In another variation, (SFI), (SFII), (SFIII)(B)(vii), (SFIV)(A), (SFV)(B), and (SFVI)(H)(ii) apply. In another variation, (SFI), (SFII), (SFIII)(B)(vii), (SFIV)(B), (SFV)(B), and (SFVI)(H)(ii) apply. In another variation, (SFI), (SFII), (SFIII)(B)(vii), (SFIV)(C), (SFV)(B), and (SFVI)(H)(ii) apply. In another variation, (SFI), (SFII), (SFIII)(B)(vii), (SFIV)(D), (SFV)(B), and (SFVI)(H)(ii) apply. In another variation, (SFI), (SFII), (SFIII)(B)(vii), (SFIV)(E), (SFV)(B), and (SFVI)(H)(ii) apply. In another variation, (SFI), (SFII), (SFIII)(B)(vii), (SFIV)(F), (SFV)(B), and (SFVI)(H)(ii) apply. In another variation, (SFI), (SFII), (SFIII)(B)(vii), (SFIV)(G), (SFV)(B), and (SFVI)(H)(ii) apply. In another variation, (SFI), (SFII), (SFIII)(B)(vii), (SFIV)(H), (SFV)(B), and (SFVI)(H)(ii) apply. In another variation, (SFI), (SFII), (SFIII)(B)(xvi), (SFIV)(A), (SFV)(B), and (SFVI)(H)(ii) apply. In another variation, (SFI), (SFII), (SFIII)(B)(xvi), (SFIV)(B), (SFV)(B), and (SFVI)(H)(ii) apply. In another variation, (SFI), (SFII), (SFIII)(B)(xvi), (SFIV)(C), (SFV)(B), and (SFVI)(H)(ii) apply. In another variation, (SFI), (SFII), (SFIII)(B)(xvi), (SFIV)(D), (SFV)(B), and (SFVI)(H)(ii) apply. In another variation, (SFI), (SFII), (SFIII)(B)(xvi), (SFIV)(E), (SFV)(B), and (SFVI)(H)(ii) apply. In another variation, (SFI), (SFII), (SFIII)(B)(xvi), (SFIV)(F), (SFV)(B), and (SFVI)(H)(ii) apply. In another variation, (SFI), (SFII), (SFIII)(B)(xvi), (SFIV)(G), (SFV)(B), and (SFVI)(H)(ii) apply. In another variation, (SFI), (SFII), (SFIII)(B)(xvi), (SFIV)(H), (SFV)(B), and (SFVI)(H)(ii) apply. In another variation, (SFI), (SFII), (SFIII)(B)(v), (SFIV)(B), (SFV)(B), and (SFVI)(H)(ii) apply. In another variation, (SFI), (SFII), (SFIII)(B)(viii), (SFIV)(B), (SFV)(B), and (SFVI)(H)(ii) apply. In another variation, (SFI), (SFII), (SFIII)(B)(x), (SFIV)(B), (SFV)(B), and (SFVI)(H)(ii) apply. In another variation, (SFI), (SFII), (SFIII)(B)(xii), (SFIV)(B), (SFV)(B), and (SFVI)(H)(ii) apply. In another variation, (SFI), (SFII), (SFIII)(B)(xiv), (SFIV)(B), (SFV)(B), and (SFVI)(H)(ii) apply. In another variation, (SFI), (SFII), (SFIII)(B)(xv), (SFIV)(B), (SFV)(B), and (SFVI)(H)(ii) apply. In another variation, (SFI), (SFII), (SFIII)(B)(xvii), (SFIV)(B), (SFV)(B), and (SFVI)(H)(ii) apply. In another variation, (SFI), (SFII), (SFIII)(B)(xviii), (SFIV)(B), (SFV)(B), and (SFVI)(H)(ii) apply.

In another variation, (SFI), (SFII), (SFIII)(A)(i), (SFV)(B), and (SFVI)(H)(v) apply. In another variation, (SFI), (SFII), (SFIII)(A)(ii), (SFV)(B), and (SFVI)(H)(v) apply. In another variation, (SFI), (SFII), (SFIII)(A)(iii), (SFV)(B), and (SFVI)(H)(v) apply. In another variation, (SFI), (SFII), (SFIII)(A)(iv), (SFV)(B), and (SFVI)(H)(v) apply. In another variation, (SFI), (SFII), (SFIII)(A)(v), (SFV)(B), and (SFVI)(H)(v) apply. In another variation, (SFI), (SFII), (SFIII)(A)(vi), (SFV)(B), and (SFVI)(H)(v) apply. In another variation, (SFI), (SFII), (SFIII)(A)(vii), (SFV)(B), and (SFVI)(H)(v) apply. In another variation, (SFI), (SFII), (SFIII)(A)(viii), (SFV)(B), and (SFVI)(H)(v) apply. In another variation, (SFI), (SFII), (SFIII)(A)(ix), (SFV)(B), and (SFVI)(H)(v) apply. In another variation, (SFI), (SFII), (SFIII)(A)(x), (SFV)(B), and (SFVI)(H)(v) apply. In another variation, (SFI), (SFII), (SFIII)(A)(xi), (SFV)(B), and (SFVI)(H)(v) apply. In another variation, (SFI), (SFII), (SFIII)(A)(xii), (SFV)(B), and (SFVI)(H)(v) apply. In another variation, (SFI), (SFII), (SFIII)(A)(xiii), (SFV)(B), and (SFVI)(H)(v) apply. In another variation, (SFI), (SFII), (SFIII)(B)(ii), (SFIV)(A), (SFV)(B), and (SFVI)(H)(v) apply. In another variation, (SFI), (SFII), (SFIII)(B)(ii), (SFIV)(B), (SFV)(B), and (SFVI)(H)(v) apply. In another variation, (SFI), (SFII), (SFIII)(B)(ii), (SFIV)(C), (SFV)(B), and (SFVI)(H)(v) apply. In another variation, (SFI), (SFII), (SFIII)(B)(ii), (SFIV)(D), (SFV)(B), and (SFVI)(H)(v) apply. In another variation, (SFI), (SFII), (SFIII)(B)(ii), (SFIV)(E), (SFV)(B), and (SFVI)(H)(v) apply. In another variation, (SFI), (SFII), (SFIII)(B)(ii), (SFIV)(F), (SFV)(B), and (SFVI)(H)(v) apply. In another variation, (SFI), (SFII), (SFIII)(B)(ii), (SFIV)(G), (SFV)(B), and (SFVI)(H)(v) apply. In another variation, (SFI), (SFII), (SFIII)(B)(ii), (SFIV)(H), (SFV)(B), and (SFVI)(H)(v) apply. In another variation, (SFI), (SFII), (SFIII)(B)(iv), (SFIV)(A), (SFV)(B), and (SFVI)(H)(v) apply. In another variation, (SFI), (SFII), (SFIII)(B)(iv), (SFIV)(B), (SFV)(B), and (SFVI)(H)(v) apply. In another variation, (SFI), (SFII), (SFIII)(B)(iv), (SFIV)(C), (SFV)(B), and (SFVI)(H)(v) apply. In another variation, (SFI), (SFII), (SFIII)(B)(iv), (SFIV)(D), (SFV)(B), and (SFVI)(H)(v) apply. In another variation, (SFI), (SFII), (SFIII)(B)(iv), (SFIV)(E), (SFV)(B), and (SFVI)(H)(v) apply. In another variation, (SFI), (SFII), (SFIII)(B)(iv), (SFIV)(F), (SFV)(B), and (SFVI)(H)(v) apply. In another variation, (SFI), (SFII), (SFIII)(B)(iv), (SFIV)(G), (SFV)(B), and (SFVI)(H)(v) apply. In another variation, (SFI), (SFII), (SFIII)(B)(iv), (SFIV)(H), (SFV)(B), and (SFVI)(H)(v) apply. In another variation, (SFI), (SFII), (SFIII)(B)(vii), (SFIV)(A), (SFV)(B), and (SFVI)(H)(v) apply. In another variation, (SFI), (SFII), (SFIII)(B)(vii), (SFIV)(B), (SFV)(B), and (SFVI)(H)(v) apply. In another variation, (SFI), (SFII), (SFIII)(B)(vii), (SFIV)(C), (SFV)(B), and (SFVI)(H)(v) apply. In another variation, (SFI), (SFII), (SFIII)(B)(vii), (SFIV)(D), (SFV)(B), and (SFVI)(H)(v) apply. In another variation, (SFI), (SFII), (SFIII)(B)(vii), (SFIV)(E), (SFV)(B), and (SFVI)(H)(v) apply. In another variation, (SFI), (SFII), (SFIII)(B)(vii), (SFIV)(F), (SFV)(B), and (SFVI)(H)(v) apply. In another variation, (SFI), (SFII), (SFIII)(B)(vii), (SFIV)(G), (SFV)(B), and (SFVI)(H)(v) apply. In another variation, (SFI), (SFII), (SFIII)(B)(vii), (SFIV)(H), (SFV)(B), and (SFVI)(H)(v) apply. In another variation, (SFI), (SFII), (SFIII)(B)(xvi), (SFIV)(A), (SFV)(B), and (SFVI)(H)(v) apply. In another variation, (SFI), (SFII), (SFIII)(B)(xvi), (SFIV)(B), (SFV)(B), and (SFVI)(H)(v) apply. In another variation, (SFI), (SFII), (SFIII)(B)(xvi), (SFIV)(C), (SFV)(B), and (SFVI)(H)(v) apply. In another variation, (SFI), (SFII), (SFIII)(B)(xvi), (SFIV)(D), (SFV)(B), and (SFVI)(H)(v) apply. In another variation, (SFI), (SFII), (SFIII)(B)(xvi), (SFIV)(E), (SFV)(B), and (SFVI)(H)(v) apply. In another variation, (SFI), (SFII), (SFIII)(B)(xvi), (SFIV)(F), (SFV)(B), and (SFVI)(H)(v) apply. In another variation, (SFI), (SFII), (SFIII)(B)(xvi), (SFIV)(G), (SFV)(B), and (SFVI)(H)(v) apply. In another variation, (SFI), (SFII), (SFIII)(B)(xvi), (SFIV)(H), (SFV)(B), and (SFVI)(H)(v) apply. In another variation, (SFI), (SFII), (SFIII)(B)(v), (SFIV)(B), (SFV)(B), and (SFVI)(H)(v) apply. In another variation, (SFI), (SFII), (SFIII)(B)(viii), (SFIV)(B), (SFV)(B), and (SFVI)(H)(v) apply. In another variation, (SFI), (SFII), (SFIII)(B)(x), (SFIV)(B), (SFV)(B), and (SFVI)(H)(v) apply. In another variation, (SFI), (SFII), (SFIII)(B)(xii), (SFIV)(B), (SFV)(B), and (SFVI)(H)(v) apply. In another variation, (SFI), (SFII), (SFIII)(B)(xiv), (SFIV)(B), (SFV)(B), and (SFVI)(H)(v) apply. In another variation, (SFI), (SFII), (SFIII)(B)(xv), (SFIV)(B), (SFV)(B), and (SFVI)(H)(v) apply. In another variation, (SFI), (SFII), (SFIII)(B)(xvii), (SFIV)(B), (SFV)(B), and (SFVI)(H)(v) apply. In another variation, (SFI), (SFII), (SFIII)(B)(xviii), (SFIV)(B), (SFV)(B), and (SFVI)(H)(v) apply.

In another variation, (SFI), (SFII), (SFIII)(A)(i), (SFV)(B), and (SFVI)(H)(vi) apply. In another variation, (SFI), (SFII), (SFIII)(A)(ii), (SFV)(B), and (SFVI)(H)(vi) apply. In another variation, (SFI), (SFII), (SFIII)(A)(iii), (SFV)(B), and (SFVI)(H)(vi) apply. In another variation, (SFI), (SFII), (SFIII)(A)(iv), (SFV)(B), and (SFVI)(H)(vi) apply. In another variation, (SFI), (SFII), (SFIII)(A)(v), (SFV)(B), and (SFVI)(H)(vi) apply. In another variation, (SFI), (SFII), (SFIII)(A)(vi), (SFV)(B), and (SFVI)(H)(vi) apply. In another variation, (SFI), (SFII), (SFIII)(A)(vii), (SFV)(B), and (SFVI)(H)(vi) apply. In another variation, (SFI), (SFII), (SFIII)(A)(viii), (SFV)(B), and (SFVI)(H)(vi) apply. In another variation, (SFI), (SFII), (SFIII)(A)(ix), (SFV)(B), and (SFVI)(H)(vi) apply. In another variation, (SFI), (SFII), (SFIII)(A)(x), (SFV)(B), and (SFVI)(H)(vi) apply. In another variation, (SFI), (SFII), (SFIII)(A)(xi), (SFV)(B), and (SFVI)(H)(vi) apply. In another variation, (SFI), (SFII), (SFIII)(A)(xii), (SFV)(B), and (SFVI)(H)(vi) apply. In another variation, (SFI), (SFII), (SFIII)(A)(xiii), (SFV)(B), and (SFVI)(H)(vi) apply. In another variation, (SFI), (SFII), (SFIII)(B)(ii), (SFIV)(A), (SFV)(B), and (SFVI)(H)(vi) apply. In another variation, (SFI), (SFII), (SFIII)(B)(ii), (SFIV)(B), (SFV)(B), and (SFVI)(H)(vi) apply. In another variation, (SFI), (SFII), (SFIII)(B)(ii), (SFIV)(C), (SFV)(B), and (SFVI)(H)(vi) apply. In another variation, (SFI), (SFII), (SFIII)(B)(ii), (SFIV)(D), (SFV)(B), and (SFVI)(H)(vi) apply. In another variation, (SFI), (SFII), (SFIII)(B)(ii), (SFIV)(E), (SFV)(B), and (SFVI)(H)(vi) apply. In another variation, (SFI), (SFII), (SFIII)(B)(ii), (SFIV)(F), (SFV)(B), and (SFVI)(H)(vi) apply. In another variation, (SFI), (SFII), (SFIII)(B)(ii), (SFIV)(G), (SFV)(B), and (SFVI)(H)(vi) apply. In another variation, (SFI), (SFII), (SFIII)(B)(ii), (SFIV)(H), (SFV)(B), and (SFVI)(H)(vi) apply. In another variation, (SFI), (SFII), (SFIII)(B)(iv), (SFIV)(A), (SFV)(B), and (SFVI)(H)(vi) apply. In another variation, (SFI), (SFII), (SFIII)(B)(iv), (SFIV)(B), (SFV)(B), and (SFVI)(H)(vi) apply. In another variation, (SFI), (SFII), (SFIII)(B)(iv), (SFIV)(C), (SFV)(B), and (SFVI)(H)(vi) apply. In another variation, (SFI), (SFII), (SFIII)(B)(iv), (SFIV)(D), (SFV)(B), and (SFVI)(H)(vi) apply. In another variation, (SFI), (SFII), (SFIII)(B)(iv), (SFIV)(E), (SFV)(B), and (SFVI)(H)(vi) apply. In another variation, (SFI), (SFII), (SFIII)(B)(iv), (SFIV)(F), (SFV)(B), and (SFVI)(H)(vi) apply. In another variation, (SFI), (SFII), (SFIII)(B)(iv), (SFIV)(G), (SFV)(B), and (SFVI)(H)(vi) apply. In another variation, (SFI), (SFII), (SFIII)(B)(iv), (SFIV)(H), (SFV)(B), and (SFVI)(H)(vi) apply. In another variation, (SFI), (SFII), (SFIII)(B)(vii), (SFIV)(A), (SFV)(B), and (SFVI)(H)(vi) apply. In another variation, (SFI), (SFII), (SFIII)(B)(vii), (SFIV)(B), (SFV)(B), and (SFVI)(H)(vi) apply. In another variation, (SFI), (SFII), (SFIII)(B)(vii), (SFIV)(C), (SFV)(B), and (SFVI)(H)(vi) apply. In another variation, (SFI), (SFII), (SFIII)(B)(vii), (SFIV)(D), (SFV)(B), and (SFVI)(H)(vi) apply. In another variation, (SFI), (SFII), (SFIII)(B)(vii), (SFIV)(E), (SFV)(B), and (SFVI)(H)(vi) apply. In another variation, (SFI), (SFII), (SFIII)(B)(vii), (SFIV)(F), (SFV)(B), and (SFVI)(H)(vi) apply. In another variation, (SFI), (SFII), (SFIII)(B)(vii), (SFIV)(G), (SFV)(B), and (SFVI)(H)(vi) apply. In another variation, (SFI), (SFII), (SFIII)(B)(vii), (SFIV)(H), (SFV)(B), and (SFVI)(H)(vi) apply. In another variation, (SFI), (SFII), (SFIII)(B)(xvi), (SFIV)(A), (SFV)(B), and (SFVI)(H)(vi) apply. In another variation, (SFI), (SFII), (SFIII)(B)(xvi), (SFIV)(B), (SFV)(B), and (SFVI)(H)(vi) apply. In another variation, (SFI), (SFII), (SFIII)(B)(xvi), (SFIV)(C), (SFV)(B), and (SFVI)(H)(vi) apply. In another variation, (SFI), (SFII), (SFIII)(B)(xvi), (SFIV)(D), (SFV)(B), and (SFVI)(H)(vi) apply. In another variation, (SFI), (SFII), (SFIII)(B)(xvi), (SFIV)(E), (SFV)(B), and (SFVI)(H)(vi) apply. In another variation, (SFI), (SFII), (SFIII)(B)(xvi), (SFIV)(F), (SFV)(B), and (SFVI)(H)(vi) apply. In another variation, (SFI), (SFII), (SFIII)(B)(xvi), (SFIV)(G), (SFV)(B), and (SFVI)(H)(vi) apply. In another variation, (SFI), (SFII), (SFIII)(B)(xvi), (SFIV)(H), (SFV)(B), and (SFVI)(H)(vi) apply. In another variation, (SFI), (SFII), (SFIII)(B)(v), (SFIV)(B), (SFV)(B), and (SFVI)(H)(vi) apply. In another variation, (SFI), (SFII), (SFIII)(B)(viii), (SFIV)(B), (SFV)(B), and (SFVI)(H)(vi) apply. In another variation, (SFI), (SFII), (SFIII)(B)(x), (SFIV)(B), (SFV)(B), and (SFVI)(H)(vi) apply. In another variation, (SFI), (SFII), (SFIII)(B)(xii), (SFIV)(B), (SFV)(B), and (SFVI)(H)(vi) apply. In another variation, (SFI), (SFII), (SFIII)(B)(xiv), (SFIV)(B), (SFV)(B), and (SFVI)(H)(vi) apply. In another variation, (SFI), (SFII), (SFIII)(B)(xv), (SFIV)(B), (SFV)(B), and (SFVI)(H)(vi) apply. In another variation, (SFI), (SFII), (SFIII)(B)(xvii), (SFIV)(B), (SFV)(B), and (SFVI)(H)(vi) apply. In another variation, (SFI), (SFII), (SFIII)(B)(xviii), (SFIV)(B), (SFV)(B), and (SFVI)(H)(vi) apply.

In another variation, (SFI), (SFII), (SFIII)(A)(i), (SFV)(B), and (SFVI)(H)(vii) apply. In another variation, (SFI), (SFII), (SFIII)(A)(ii), (SFV)(B), and (SFVI)(H)(vii) apply. In another variation, (SFI), (SFII), (SFIII)(A)(iii), (SFV)(B), and (SFVI)(H)(vii) apply. In another variation, (SFI), (SFII), (SFIII)(A)(iv), (SFV)(B), and (SFVI)(H)(vii) apply. In another variation, (SFI), (SFII), (SFIII)(A)(v), (SFV)(B), and (SFVI)(H)(vii) apply. In another variation, (SFI), (SFII), (SFIII)(A)(vi), (SFV)(B), and (SFVI)(H)(vii) apply. In another variation, (SFI), (SFII), (SFIII)(A)(vii), (SFV)(B), and (SFVI)(H)(vii) apply. In another variation, (SFI), (SFII), (SFIII)(A)(viii), (SFV)(B), and (SFVI)(H)(vii) apply. In another variation, (SFI), (SFII), (SFIII)(A)(ix), (SFV)(B), and (SFVI)(H)(vii) apply. In another variation, (SFI), (SFII), (SFIII)(A)(x), (SFV)(B), and (SFVI)(H)(vii) apply. In another variation, (SFI), (SFII), (SFIII)(A)(xi), (SFV)(B), and (SFVI)(H)(vii) apply. In another variation, (SFI), (SFII), (SFIII)(A)(xii), (SFV)(B), and (SFVI)(H)(vii) apply. In another variation, (SFI), (SFII), (SFIII)(A)(xiii), (SFV)(B), and (SFVI)(H)(vii) apply. In another variation, (SFI), (SFII), (SFIII)(B)(ii), (SFIV)(A), (SFV)(B), and (SFVI)(H)(vii) apply. In another variation, (SFI), (SFII), (SFIII)(B)(ii), (SFIV)(B), (SFV)(B), and (SFVI)(H)(vii) apply. In another variation, (SFI), (SFII), (SFIII)(B)(ii), (SFIV)(C), (SFV)(B), and (SFVI)(H)(vii) apply. In another variation, (SFI), (SFII), (SFIII)(B)(ii), (SFIV)(D), (SFV)(B), and (SFVI)(H)(vii) apply. In another variation, (SFI), (SFII), (SFIII)(B)(ii), (SFIV)(E), (SFV)(B), and (SFVI)(H)(vii) apply. In another variation, (SFI), (SFII), (SFIII)(B)(ii), (SFIV)(F), (SFV)(B), and (SFVI)(H)(vii) apply. In another variation, (SFI), (SFII), (SFIII)(B)(ii), (SFIV)(G), (SFV)(B), and (SFVI)(H)(vii) apply. In another variation, (SFI), (SFII), (SFIII)(B)(ii), (SFIV)(H), (SFV)(B), and (SFVI)(H)(vii) apply. In another variation, (SFI), (SFII), (SFIII)(B)(iv), (SFIV)(A), (SFV)(B), and (SFVI)(H)(vii) apply. In another variation, (SFI), (SFII), (SFIII)(B)(iv), (SFIV)(B), (SFV)(B), and (SFVI)(H)(vii) apply. In another variation, (SFI), (SFII), (SFIII)(B)(iv), (SFIV)(C), (SFV)(B), and (SFVI)(H)(vii) apply. In another variation, (SFI), (SFII), (SFIII)(B)(iv), (SFIV)(D), (SFV)(B), and (SFVI)(H)(vii) apply. In another variation, (SFI), (SFII), (SFIII)(B)(iv), (SFIV)(E), (SFV)(B), and (SFVI)(H)(vii) apply. In another variation, (SFI), (SFII), (SFIII)(B)(iv), (SFIV)(F), (SFV)(B), and (SFVI)(H)(vii) apply. In another variation, (SFI), (SFII), (SFIII)(B)(iv), (SFIV)(G), (SFV)(B), and (SFVI)(H)(vii) apply. In another variation, (SFI), (SFII), (SFIII)(B)(iv), (SFIV)(H), (SFV)(B), and (SFVI)(H)(vii) apply. In another variation, (SFI), (SFII), (SFIII)(B)(vii), (SFIV)(A), (SFV)(B), and (SFVI)(H)(vii) apply. In another variation, (SFI), (SFII), (SFIII)(B)(vii), (SFIV)(B), (SFV)(B), and (SFVI)(H)(vii) apply. In another variation, (SFI), (SFII), (SFIII)(B)(vii), (SFIV)(C), (SFV)(B), and (SFVI)(H)(vii) apply. In another variation, (SFI), (SFII), (SFIII)(B)(vii), (SFIV)(D), (SFV)(B), and (SFVI)(H)(vii) apply. In another variation, (SFI), (SFII), (SFIII)(B)(vii), (SFIV)(E), (SFV)(B), and (SFVI)(H)(vii) apply. In another variation, (SFI), (SFII), (SFIII)(B)(vii), (SFIV)(F), (SFV)(B), and (SFVI)(H)(vii) apply. In another variation, (SFI), (SFII), (SFIII)(B)(vii), (SFIV)(G), (SFV)(B), and (SFVI)(H)(vii) apply. In another variation, (SFI), (SFII), (SFIII)(B)(vii), (SFIV)(H), (SFV)(B), and (SFVI)(H)(vii) apply. In another variation, (SFI), (SFII), (SFIII)(B)(xvi), (SFIV)(A), (SFV)(B), and (SFVI)(H)(vii) apply. In another variation, (SFI), (SFII), (SFIII)(B)(xvi), (SFIV)(B), (SFV)(B), and (SFVI)(H)(vii) apply. In another variation, (SFI), (SFII), (SFIII)(B)(xvi), (SFIV)(C), (SFV)(B), and (SFVI)(H)(vii) apply. In another variation, (SFI), (SFII), (SFIII)(B)(xvi), (SFIV)(D), (SFV)(B), and (SFVI)(H)(vii) apply. In another variation, (SFI), (SFII), (SFIII)(B)(xvi), (SFIV)(E), (SFV)(B), and (SFVI)(H)(vii) apply. In another variation, (SFI), (SFII), (SFIII)(B)(xvi), (SFIV)(F), (SFV)(B), and (SFVI)(H)(vii) apply. In another variation, (SFI), (SFII), (SFIII)(B)(xvi), (SFIV)(G), (SFV)(B), and (SFVI)(H)(vii) apply. In another variation, (SFI), (SFII), (SFIII)(B)(xvi), (SFIV)(H), (SFV)(B), and (SFVI)(H)(vii) apply. In another variation, (SFI), (SFII), (SFIII)(B)(v), (SFIV)(B), (SFV)(B), and (SFVI)(H)(vii) apply. In another variation, (SFI), (SFII), (SFIII)(B)(viii), (SFIV)(B), (SFV)(B), and (SFVI)(H)(vii) apply. In another variation, (SFI), (SFII), (SFIII)(B)(x), (SFIV)(B), (SFV)(B), and (SFVI)(H)(vii) apply. In another variation, (SFI), (SFII), (SFIII)(B)(xii), (SFIV)(B), (SFV)(B), and (SFVI)(H)(vii) apply. In another variation, (SFI), (SFII), (SFIII)(B)(xiv), (SFIV)(B), (SFV)(B), and (SFVI)(H)(vii) apply. In another variation, (SFI), (SFII), (SFIII)(B)(xv), (SFIV)(B), (SFV)(B), and (SFVI)(H)(vii) apply. In another variation, (SFI), (SFII), (SFIII)(B)(xvii), (SFIV)(B), (SFV)(B), and (SFVI)(H)(vii) apply. In another variation, (SFI), (SFII), (SFIII)(B)(xviii), (SFIV)(B), (SFV)(B), and (SFVI)(H)(vii) apply.

Any variations or combinations recited herein for compounds of formula (I) also apply to formula (A), with the addition of any possible combinations of R¹⁵ and R¹⁶.

Representative compounds are listed in FIG. 1.

In some embodiments, provided is a compound selected from Compound Nos. 1-66 in FIG. 1, or a stereoisomer thereof (including a mixture of two or more stereoisomers thereof), or a salt thereof. In some embodiments, the compound is a salt of a compound selected from Compound Nos. 1-66 in FIG. 1, or a stereoisomer thereof.

In some embodiments, provided is a compound selected from Compound Nos. 1-147, or a stereoisomer thereof (including a mixture of two or more stereoisomers thereof), or a salt thereof. In some embodiments, the compound is a salt of a compound selected from Compound Nos. 1-147, or a stereoisomer thereof.

In some embodiments, provided is a compound selected from Compound Nos. 1-665, or a stereoisomer thereof (including a mixture of two or more stereoisomers thereof), or a salt thereof. In some embodiments, the compound is a salt of a compound selected from Compound Nos. 1-665, or a stereoisomer thereof.

In some embodiments, provided is a compound selected from Compound Nos. 1-780, or a stereoisomer thereof (including a mixture of two or more stereoisomers thereof), or a salt thereof. In some embodiments, the compound is a salt of a compound selected from Compound Nos. 1-780, or a stereoisomer thereof.

In one variation, the compound detailed herein is selected from the group consisting of:
4-(cyclopropyl(4-(5,6,7,8-tetrahydro-1,8-naphthyridin-2-yl)butyl)amino)-2-((6-(difluoromethyl)pyrimidin-4-yl)amino)butanoic acid;
4-(cyclopropyl(4-(5,6,7,8-tetrahydro-1,8-naphthyridin-2-yl)butyl)amino)-2-(pyrimidin-4-ylamino)butanoic acid;
4-(cyclopropyl(4-(5,6,7,8-tetrahydro-1,8-naphthyridin-2-yl)butyl)amino)-2-((1-methyl-1H-pyrazolo[3,4-d]pyrimidin-4-yl)amino)butanoic acid;
4-((2-hydroxy-2-methylpropyl)(4-(5,6,7,8-tetrahydro-1,8-naphthyridin-2-yl)butyl)amino)-2-(pyrimidin-4-ylamino)butanoic acid;
4-((2-methoxyethyl)(4-(5,6,7,8-tetrahydro-1,8-naphthyridin-2-yl)butyl)amino)-2-(quinazolin-4-ylamino)butanoic acid;
4-(cyclopropyl(4-(5,6,7,8-tetrahydro-1,8-naphthyridin-2-yl)butyl)amino)-2-(quinazolin-4-ylamino)butanoic acid;
2-((7-fluoroquinazolin-4-yl)amino)-4-((2-methoxyethyl)(4-(5,6,7,8-tetrahydro-1,8-naphthyridin-2-yl)butyl)amino)butanoic acid;
4-((2,2-difluoroethyl)(4-(5,6,7,8-tetrahydro-1,8-naphthyridin-2-yl)butyl)amino)-2-(quinazolin-4-ylamino)butanoic acid;
4-((3,3-difluorocyclobutyl)(4-(5,6,7,8-tetrahydro-1,8-naphthyridin-2-yl)butyl)amino)-2-(quinazolin-4-ylamino)butanoic acid;
4-((2-methoxyethyl)(4-(5,6,7,8-tetrahydro-1,8-naphthyridin-2-yl)butyl)amino)-2-((2-methylquinazolin-4-yl)amino)butanoic acid;
4-((2-methoxyethyl)(4-(5,6,7,8-tetrahydro-1,8-naphthyridin-2-yl)butyl)amino)-2-(pyrido[2,3-d]pyrimidin-4-ylamino)butanoic acid;
2-((7-fluoro-2-methylquinazolin-4-yl)amino)-4-((2-methoxyethyl)(4-(5,6,7,8-tetrahydro-1,8-naphthyridin-2-yl)butyl)amino)butanoic acid;
4-((2-methoxyethyl)(4-(5,6,7,8-tetrahydro-1,8-naphthyridin-2-yl)butyl)amino)-2-((7-(trifluoromethyl)quinazolin-4-yl)amino)butanoic acid;
4-((2-methoxyethyl)(4-(5,6,7,8-tetrahydro-1,8-naphthyridin-2-yl)butyl)amino)-2-((2-(trifluoromethyl)quinazolin-4-yl)amino)butanoic acid;
4-((2-methoxyethyl)(4-(5,6,7,8-tetrahydro-1,8-naphthyridin-2-yl)butyl)amino)-2-((8-(trifluoromethyl)quinazolin-4-yl)amino)butanoic acid;
4-((2-methoxyethyl)(4-(5,6,7,8-tetrahydro-1,8-naphthyridin-2-yl)butyl)amino)-2-(pyrido[3,2-d]pyrimidin-4-ylamino)butanoic acid;
4-((2-methoxyethyl)(4-(5,6,7,8-tetrahydro-1,8-naphthyridin-2-yl)butyl)amino)-2-(pyrido[3,4-d]pyrimidin-4-ylamino)butanoic acid;
2-((5-fluoroquinazolin-4-yl)amino)-4-((2-methoxyethyl)(4-(5,6,7,8-tetrahydro-1,8-naphthyridin-2-yl)butyl)amino)butanoic acid;
2-((6-fluoroquinazolin-4-yl)amino)-4-((2-methoxyethyl)(4-(5,6,7,8-tetrahydro-1,8-naphthyridin-2-yl)butyl)amino)butanoic acid;
2-((8-fluoroquinazolin-4-yl)amino)-4-((2-methoxyethyl)(4-(5,6,7,8-tetrahydro-1,8-naphthyridin-2-yl)butyl)amino)butanoic acid;
2-((6,7-difluoroquinazolin-4-yl)amino)-4-((2-methoxyethyl)(4-(5,6,7,8-tetrahydro-1,8-naphthyridin-2-yl)butyl)amino)butanoic acid;
4-((2-methoxyethyl)(4-(5,6,7,8-tetrahydro-1,8-naphthyridin-2-yl)butyl)amino)-2-((2-methyl-6-(trifluoromethyl)pyrimidin-4-yl)amino)butanoic acid;
2-((6-(difluoromethyl)pyrimidin-4-yl)amino)-4-((2-methoxyethyl)(4-(5,6,7,8-tetrahydro-1,8-naphthyridin-2-yl)butyl)amino)butanoic acid;
4-((2-methoxyethyl)(4-(5,6,7,8-tetrahydro-1,8-naphthyridin-2-yl)butyl)amino)-2-((2-(trifluoromethyl)pyrimidin-4-yl)amino)butanoic acid;
4-((2-methoxypropyl)(4-(5,6,7,8-tetrahydro-1,8-naphthyridin-2-yl)butyl)amino)-2-(quinazolin-4-ylamino)butanoic acid;
4-((2-methoxyethyl)(4-(5,6,7,8-tetrahydro-1,8-naphthyridin-2-yl)butyl)amino)-2-((6-methyl-2-(trifluoromethyl)pyrimidin-4-yl)amino)butanoic acid;
4-((2-(methylsulfonyl)ethyl)(4-(5,6,7,8-tetrahydro-1,8-naphthyridin-2-yl)butyl)amino)-2-(quinazolin-4-ylamino)butanoic acid;
4-((2-phenoxyethyl)(4-(5,6,7,8-tetrahydro-1,8-naphthyridin-2-yl)butyl)amino)-2-(quinazolin-4-ylamino)butanoic acid;
4-((3,3-difluoropropyl)(4-(5,6,7,8-tetrahydro-1,8-naphthyridin-2-yl)butyl)amino)-2-(quinazolin-4-ylamino)butanoic acid;
4-((3-fluoropropyl)(4-(5,6,7,8-tetrahydro-1,8-naphthyridin-2-yl)butyl)amino)-2-(quinazolin-4-ylamino)butanoic acid;
4-((2-fluoro-3-methoxypropyl)(4-(5,6,7,8-tetrahydro-1,8-naphthyridin-2-yl)butyl)amino)-2-(quinazolin-4-ylamino)butanoic acid;
2-((7-fluoro-2-methylquinazolin-4-yl)amino)-4-((2-fluoro-3-methoxypropyl)(4-(5,6,7,8-tetrahydro-1,8-naphthyridin-2-yl)butyl)amino)butanoic acid;
4-(((3,3-difluorocyclobutyl)methyl)(4-(5,6,7,8-tetrahydro-1,8-naphthyridin-2-yl)butyl)amino)-2-((7-fluoro-2-methylquinazolin-4-yl)amino)butanoic acid;
2-(isoquinolin-1-ylamino)-4-((2-methoxyethyl)(4-(5,6,7,8-tetrahydro-1,8-naphthyridin-2-yl)butyl)amino)butanoic acid;
4-((2-(difluoromethoxy)ethyl)(4-(5,6,7,8-tetrahydro-1,8-naphthyridin-2-yl)butyl)amino)-2-(quinazolin-4-ylamino)butanoic acid;
4-((2-methoxyethyl)(4-(5,6,7,8-tetrahydro-1,8-naphthyridin-2-yl)butyl)amino)-2-(quinolin-4-ylamino)butanoic acid;
2-((7-chloroquinazolin-4-yl)amino)-4-((2-methoxyethyl)(4-(5,6,7,8-tetrahydro-1,8-naphthyridin-2-yl)butyl)amino)butanoic acid;
2-((8-chloroquinazolin-4-yl)amino)-4-((2-methoxyethyl)(4-(5,6,7,8-tetrahydro-1,8-naphthyridin-2-yl)butyl)amino)butanoic acid;
2-(quinazolin-4-ylamino)-4-((4-(5,6,7,8-tetrahydro-1,8-naphthyridin-2-yl)butyl)(2-(2,2,2-trifluoroethoxy)ethyl)amino)butanoic acid;
2-((7-fluoro-2-methylquinazolin-4-yl)amino)-4-((2-(4-fluorophenoxy)ethyl)(4-(5,6,7,8-tetrahydro-1,8-naphthyridin-2-yl)butyl)amino)butanoic acid;
4-((3-fluoropropyl)(4-(5,6,7,8-tetrahydro-1,8-naphthyridin-2-yl)butyl)amino)-2-((7-methoxyquinazolin-4-yl)amino)butanoic acid;
4-((2-(2,2-difluorocyclopropoxy)ethyl)(4-(5,6,7,8-tetrahydro-1,8-naphthyridin-2-yl)butyl)amino)-2-((7-fluoro-2-methylquinazolin-4-yl)amino)butanoic acid;
4-((3-fluoropropyl)(4-(5,6,7,8-tetrahydro-1,8-naphthyridin-2-yl)butyl)amino)-2-((8-methoxyquinazolin-4-yl)amino)butanoic acid;
2-((6-(1H-pyrazol-1-yl)pyrimidin-4-yl)amino)-4-((2-methoxyethyl)(4-(5,6,7,8-tetrahydro-1,8-naphthyridin-2-yl)butyl)amino)butanoic acid;
4-((2-(3,5-dimethyl-1H-pyrazol-1-yl)ethyl)(4-(5,6,7,8-tetrahydro-1,8-naphthyridin-2-yl)butyl)amino)-2-(quinazolin-4-ylamino)butanoic acid;
4-(((S)-2-fluoro-3-methoxypropyl)(4-(5,6,7,8-tetrahydro-1,8-naphthyridin-2-yl)butyl)amino)-2-((2-methylquinazolin-4-yl)amino)butanoic acid;
4-((2-(3,5-difluorophenoxy)ethyl)(4-(5,6,7,8-tetrahydro-1,8-naphthyridin-2-yl)butyl)amino)-2-(quinazolin-4-ylamino)butanoic acid;
2-((8-chloroquinazolin-4-yl)amino)-4-((2-(pyridin-2-yloxy)ethyl)(4-(5,6,7,8-tetrahydro-1,8-naphthyridin-2-yl)butyl)amino)butanoic acid;
4-((2-(pyridin-2-yloxy)ethyl)(4-(5,6,7,8-tetrahydro-1,8-naphthyridin-2-yl)butyl)amino)-2-(quinazolin-4-ylamino)butanoic acid;
4-((2-(2,2-difluoroethoxy)ethyl)(4-(5,6,7,8-tetrahydro-1,8-naphthyridin-2-yl)butyl)amino)-2-(quinazolin-4-ylamino)butanoic acid;
2-(pyrido[3,2-d]pyrimidin-4-ylamino)-4-((4-(5,6,7,8-tetrahydro-1,8-naphthyridin-2-yl)butyl)(2-(2,2,2-trifluoroethoxy)ethyl)amino)butanoic acid;
4-((2-((2-methylpyridin-3-yl)oxy)ethyl)(4-(5,6,7,8-tetrahydro-1,8-naphthyridin-2-yl)butyl)amino)-2-(quinazolin-4-ylamino)butanoic acid;
2-((7-fluoro-2-methylquinazolin-4-yl)amino)-4-((2-((2-methylpyridin-3-yl)oxy)ethyl)(4-(5,6,7,8-tetrahydro-1,8-naphthyridin-2-yl)butyl)amino)butanoic acid;
4-((2-((2-methylpyridin-3-yl)oxy)ethyl)(4-(5,6,7,8-tetrahydro-1,8-naphthyridin-2-yl)butyl)amino)-2-(pyrido[3,2-d]pyrimidin-4-ylamino)butanoic acid;
4-((2-ethoxyethyl)(4-(5,6,7,8-tetrahydro-1,8-naphthyridin-2-yl)butyl)amino)-2-(quinazolin-4-ylamino)butanoic acid;
2-((7-fluoro-2-methylquinazolin-4-yl)amino)-4-((2-((6-methylpyridin-3-yl)oxy)ethyl)(4-(5,6,7,8-tetrahydro-1,8-naphthyridin-2-yl)butyl)amino)butanoic acid;
4-((2-((6-methylpyridin-3-yl)oxy)ethyl)(4-(5,6,7,8-tetrahydro-1,8-naphthyridin-2-yl)butyl)amino)-2-(pyrido[3,2-d]pyrimidin-4-ylamino)butanoic acid;
4-((2-((5-fluoropyridin-3-yl)oxy)ethyl)(4-(5,6,7,8-tetrahydro-1,8-naphthyridin-2-yl)butyl)amino)-2-(quinazolin-4-ylamino)butanoic acid;
4-((2-((6-methylpyridin-3-yl)oxy)ethyl)(4-(5,6,7,8-tetrahydro-1,8-naphthyridin-2-yl)butyl)amino)-2-(quinazolin-4-ylamino)butanoic acid;
4-((2-((5-fluoropyridin-3-yl)oxy)ethyl)(4-(5,6,7,8-tetrahydro-1,8-naphthyridin-2-yl)butyl)amino)-2-(pyrido[3,2-d]pyrimidin-4-ylamino)butanoic acid;
2-((7-fluoro-2-methylquinazolin-4-yl)amino)-4-((2-((5-fluoropyridin-3-yl)oxy)ethyl)(4-(5,6,7,8-tetrahydro-1,8-naphthyridin-2-yl)butyl)amino)butanoic acid;
4-(((R)-2-methoxypropyl)(4-(5,6,7,8-tetrahydro-1,8-naphthyridin-2-yl)butyl)amino)-2-(quinazolin-4-ylamino)butanoic acid;
4-((2-acetamidoethyl)(4-(5,6,7,8-tetrahydro-1,8-naphthyridin-2-yl)butyl)amino)-2-(quinazolin-4-ylamino)butanoic acid;
4-((2-(dimethylamino)-2-oxoethyl)(4-(5,6,7,8-tetrahydro-1,8-naphthyridin-2-yl)butyl)amino)-2-(quinazolin-4-ylamino)butanoic acid;
2-((7-fluoro-2-methylquinazolin-4-yl)amino)-4-((2-methoxypropyl)(4-(5,6,7,8-tetrahydro-1,8-naphthyridin-2-yl)butyl)amino)butanoic acid; and
4-((2-methoxypropyl)(4-(5,6,7,8-tetrahydro-1,8-naphthyridin-2-yl)butyl)amino)-2-((2-methylquinazolin-4-yl)amino)butanoic acid.

In another variation, the compound detailed herein is selected from the group consisting of:
2-((3-cyanopyrazin-2-yl)amino)-4-((2-methoxyethyl)(4-(5,6,7,8-tetrahydro-1,8-naphthyridin-2-yl)butyl)amino)butanoic acid;
2-((5-cyanopyrimidin-2-yl)amino)-4-((2-methoxypropyl)(4-(5,6,7,8-tetrahydro-1,8-naphthyridin-2-yl)butyl)amino)butanoic acid;
4-((2-methoxypropyl)(4-(5,6,7,8-tetrahydro-1,8-naphthyridin-2-yl)butyl)amino)-2-((5-(trifluoromethyl)pyrimidin-2-yl)amino)butanoic acid;
2-((5-bromopyrimidin-2-yl)amino)-4-((2-methoxypropyl)(4-(5,6,7,8-tetrahydro-1,8-naphthyridin-2-yl)butyl)amino)butanoic acid;
2-((1H-pyrazolo[3,4-d]pyrimidin-4-yl)amino)-4-((2-methoxypropyl)(4-(5,6,7,8-tetrahydro-1,8-naphthyridin-2-yl)butyl)amino)butanoic acid;
4-((2-methoxypropyl)(4-(5,6,7,8-tetrahydro-1,8-naphthyridin-2-yl)butyl)amino)-2-((2-(trifluoromethyl)pyrimidin-4-yl)amino)butanoic acid;
4-((2-methoxypropyl)(4-(5,6,7,8-tetrahydro-1,8-naphthyridin-2-yl)butyl)amino)-2-((2-phenylpyrimidin-4-yl)amino)butanoic acid;
4-((2-methoxypropyl)(4-(5,6,7,8-tetrahydro-1,8-naphthyridin-2-yl)butyl)amino)-2-((1-methyl-1H-pyrazolo[3,4-d]pyrimidin-4-yl)amino)butanoic acid;
4-((2-hydroxyethyl)(4-(5,6,7,8-tetrahydro-1,8-naphthyridin-2-yl)butyl)amino)-2-(quinazolin-4-ylamino)butanoic acid;
2-((3-cyanopyrazin-2-yl)amino)-4-((2-methoxypropyl)(4-(5,6,7,8-tetrahydro-1,8-naphthyridin-2-yl)butyl)amino)butanoic acid;
2-((6-(1H-pyrazol-1-yl)pyrimidin-4-yl)amino)-4-((2-methoxypropyl)(4-(5,6,7,8-tetrahydro-1,8-naphthyridin-2-yl)butyl)amino)butanoic acid;
2-((5-fluoropyrimidin-2-yl)amino)-4-((2-methoxypropyl)(4-(5,6,7,8-tetrahydro-1,8-naphthyridin-2-yl)butyl)amino)butanoic acid;
2-((1H-pyrazolo[4,3-d]pyrimidin-7-yl)amino)-4-((2-methoxypropyl)(4-(5,6,7,8-tetrahydro-1,8-naphthyridin-2-yl)butyl)amino)butanoic acid;
4-((2-methoxypropyl)(4-(5,6,7,8-tetrahydro-1,8-naphthyridin-2-yl)butyl)amino)-2-((6-phenylpyrimidin-4-yl)amino)butanoic acid;
4-((2-methoxypropyl)(4-(5,6,7,8-tetrahydro-1,8-naphthyridin-2-yl)butyl)amino)-2-((5-phenylpyrimidin-4-yl)amino)butanoic acid;
2-((1-methyl-1H-pyrazolo[3,4-d]pyrimidin-4-yl)amino)-4-((2-phenoxyethyl)(4-(5,6,7,8-tetrahydro-1,8-naphthyridin-2-yl)butyl)amino)butanoic acid;
2-((5-bromopyrimidin-2-yl)amino)-4-((2-phenoxyethyl)(4-(5,6,7,8-tetrahydro-1,8-naphthyridin-2-yl)butyl)amino)butanoic acid;
2-((5-cyanopyrimidin-2-yl)amino)-4-((2-fluoro-3-methoxypropyl)(4-(5,6,7,8-tetrahydro-1,8-naphthyridin-2-yl)butyl)amino)butanoic acid;
4-((2-fluoro-3-methoxypropyl)(4-(5,6,7,8-tetrahydro-1,8-naphthyridin-2-yl)butyl)amino)-2-((5-(trifluoromethyl)pyrimidin-2-yl)amino)butanoic acid;
2-((5-bromopyrimidin-2-yl)amino)-4-((2-fluoro-3-methoxypropyl)(4-(5,6,7,8-tetrahydro-1,8-naphthyridin-2-yl)butyl)amino)butanoic acid;
4-((2-fluoro-3-methoxypropyl)(4-(5,6,7,8-tetrahydro-1,8-naphthyridin-2-yl)butyl)amino)-2-((2-(trifluoromethyl)pyrimidin-4-yl)amino)butanoic acid;
4-((2,2-difluoroethyl)(4-(5,6,7,8-tetrahydro-1,8-naphthyridin-2-yl)butyl)amino)-2-((1-methyl-1H-pyrazolo[3,4-d]pyrimidin-4-yl)amino)butanoic acid;
4-((2-phenoxyethyl)(4-(5,6,7,8-tetrahydro-1,8-naphthyridin-2-yl)butyl)amino)-2-((5-(trifluoromethyl)pyrimidin-2-yl)amino)butanoic acid;
2-((1H-pyrazolo[3,4-d]pyrimidin-4-yl)amino)-4-((2-phenoxyethyl)(4-(5,6,7,8-tetrahydro-1,8-naphthyridin-2-yl)butyl)amino)butanoic acid;
2-((6-(1H-pyrazol-1-yl)pyrimidin-4-yl)amino)-4-((2-phenoxyethyl)(4-(5,6,7,8-tetrahydro-1,8-naphthyridin-2-yl)butyl)amino)butanoic acid;
4-((2-phenoxyethyl)(4-(5,6,7,8-tetrahydro-1,8-naphthyridin-2-yl)butyl)amino)-2-((2-(trifluoromethyl)pyrimidin-4-yl)amino)butanoic acid;
4-((2-phenoxyethyl)(4-(5,6,7,8-tetrahydro-1,8-naphthyridin-2-yl)butyl)amino)-2-((6-phenylpyrimidin-4-yl)amino)butanoic acid;
4-((2-phenoxyethyl)(4-(5,6,7,8-tetrahydro-1,8-naphthyridin-2-yl)butyl)amino)-2-((2-(pyridin-3-yl)quinazolin-4-yl)amino)butanoic acid;
4-((2,2-difluoroethyl)(4-(5,6,7,8-tetrahydro-1,8-naphthyridin-2-yl)butyl)amino)-2-((5-(trifluoromethyl)pyrimidin-2-yl)amino)butanoic acid;
2-((5-bromopyrimidin-2-yl)amino)-4-((2,2-difluoroethyl)(4-(5,6,7,8-tetrahydro-1,8-naphthyridin-2-yl)butyl)amino)butanoic acid;
4-((2,2-difluoroethyl)(4-(5,6,7,8-tetrahydro-1,8-naphthyridin-2-yl)butyl)amino)-2-((2-(trifluoromethyl)pyrimidin-4-yl)amino)butanoic acid;
2-((6-(1H-pyrazol-1-yl)pyrimidin-4-yl)amino)-4-((2,2-difluoroethyl)(4-(5,6,7,8-tetrahydro-1,8-naphthyridin-2-yl)butyl)amino)butanoic acid;
4-((2,2-difluoroethyl)(4-(5,6,7,8-tetrahydro-1,8-naphthyridin-2-yl)butyl)amino)-2-((2-(pyridin-3-yl)quinazolin-4-yl)amino)butanoic acid;
4-((2-methoxypropyl)(4-(5,6,7,8-tetrahydro-1,8-naphthyridin-2-yl)butyl)amino)-2-((2-(pyridin-3-yl)quinazolin-4-yl)amino)butanoic acid;
2-((1-methyl-1H-pyrazolo[3,4-d]pyrimidin-4-yl)amino)-4-((2-(methylsulfonyl)ethyl)(4-(5,6,7,8-tetrahydro-1,8-naphthyridin-2-yl)butyl)amino)butanoic acid;
4-((2-(methylsulfonyl)ethyl)(4-(5,6,7,8-tetrahydro-1,8-naphthyridin-2-yl)butyl)amino)-2-((5-(trifluoromethyl)pyrimidin-2-yl)amino)butanoic acid;
2-((5-bromopyrimidin-2-yl)amino)-4-((2-(methylsulfonyl)ethyl)(4-(5,6,7,8-tetrahydro-1,8-naphthyridin-2-yl)butyl)amino)butanoic acid;
4-((2-(methylsulfonyl)ethyl)(4-(5,6,7,8-tetrahydro-1,8-naphthyridin-2-yl)butyl)amino)-2-((2-(trifluoromethyl)pyrimidin-4-yl)amino)butanoic acid;
4-((2-fluoro-3-methoxypropyl)(4-(5,6,7,8-tetrahydro-1,8-naphthyridin-2-yl)butyl)amino)-2-((1-methyl-1H-pyrazolo[3,4-d]pyrimidin-4-yl)amino)butanoic acid;
4-((2-methoxypropyl)(4-(5,6,7,8-tetrahydro-1,8-naphthyridin-2-yl)butyl)amino)-2-(pyrimidin-4-ylamino)butanoic acid;
4-((2-(methylsulfonyl)ethyl)(4-(5,6,7,8-tetrahydro-1,8-naphthyridin-2-yl)butyl)amino)-2-((2-(pyridin-3-yl)quinazolin-4-yl)amino)butanoic acid;
2-((6-(1H-pyrazol-1-yl)pyrimidin-4-yl)amino)-4-((2-fluoro-3-methoxypropyl)(4-(5,6,7,8-tetrahydro-1,8-naphthyridin-2-yl)butyl)amino)butanoic acid;
4-((2-fluoro-3-methoxypropyl)(4-(5,6,7,8-tetrahydro-1,8-naphthyridin-2-yl)butyl)amino)-2-((2-(pyridin-3-yl)quinazolin-4-yl)amino)butanoic acid;
4-((2-fluoro-3-methoxypropyl)(4-(5,6,7,8-tetrahydro-1,8-naphthyridin-2-yl)butyl)amino)-2-((5-phenylpyrimidin-4-yl)amino)butanoic acid;
2-((5-cyanopyrimidin-2-yl)amino)-4-((2-phenoxyethyl)(4-(5,6,7,8-tetrahydro-1,8-naphthyridin-2-yl)butyl)amino)butanoic acid;
2-((1H-pyrazolo[3,4-d]pyrimidin-4-yl)amino)-4-((2,2-difluoroethyl)(4-(5,6,7,8-tetrahydro-1,8-naphthyridin-2-yl)butyl)amino)butanoic acid;
4-(cyclopropyl(4-(5,6,7,8-tetrahydro-1,8-naphthyridin-2-yl)butyl)amino)-2-((5-(trifluoromethyl)pyrimidin-2-yl)amino)butanoic acid;
4-(cyclopropyl(4-(5,6,7,8-tetrahydro-1,8-naphthyridin-2-yl)butyl)amino)-2-((2-(trifluoromethyl)pyrimidin-4-yl)amino)butanoic acid;
2-((1H-pyrazolo[3,4-d]pyrimidin-4-yl)amino)-4-(cyclopropyl(4-(5,6,7,8-tetrahydro-1,8-naphthyridin-2-yl)butyl)amino)butanoic acid;
2-((5-cyclopropylpyrimidin-2-yl)amino)-4-((2-phenoxyethyl)(4-(5,6,7,8-tetrahydro-1,8-naphthyridin-2-yl)butyl)amino)butanoic acid;
2-((5-cyanopyrimidin-2-yl)amino)-4-((2,2-difluoroethyl)(4-(5,6,7,8-tetrahydro-1,8-naphthyridin-2-yl)butyl)amino)butanoic acid;
4-((2,2-difluoroethyl)(4-(5,6,7,8-tetrahydro-1,8-naphthyridin-2-yl)butyl)amino)-2-((5-phenylpyrimidin-4-yl)amino)butanoic acid;
4-((2,2-difluoroethyl)(4-(5,6,7,8-tetrahydro-1,8-naphthyridin-2-yl)butyl)amino)-2-(pyrimidin-4-ylamino)butanoic acid;
4-((2,2-difluoroethyl)(4-(5,6,7,8-tetrahydro-1,8-naphthyridin-2-yl)butyl)amino)-2-((5-fluoropyrimidin-2-yl)amino)butanoic acid;
4-((2,2-difluoroethyl)(4-(5,6,7,8-tetrahydro-1,8-naphthyridin-2-yl)butyl)amino)-2-((6-methyl-2-(pyridin-4-yl)pyrimidin-4-yl)amino)butanoic acid;
4-((2-(4-fluorophenoxy)ethyl)(4-(5,6,7,8-tetrahydro-1,8-naphthyridin-2-yl)butyl)amino)-2-((1-methyl-1H-pyrazolo[3,4-d]pyrimidin-4-yl)amino)butanoic acid;
2-((5-cyclopropylpyrimidin-2-yl)amino)-4-((2-methoxypropyl)(4-(5,6,7,8-tetrahydro-1,8-naphthyridin-2-yl)butyl)amino)butanoic acid;
2-((1H-pyrazolo[3,4-d]pyrimidin-4-yl)amino)-4-((2-(methylsulfonyl)ethyl)(4-(5,6,7,8-tetrahydro-1,8-naphthyridin-2-yl)butyl)amino)butanoic acid;
2-((6-(1H-pyrazol-1-yl)pyrimidin-4-yl)amino)-4-((2-(methylsulfonyl)ethyl)(4-(5,6,7,8-tetrahydro-1,8-naphthyridin-2-yl)butyl)amino)butanoic acid;
4-((2-fluoro-3-methoxypropyl)(4-(5,6,7,8-tetrahydro-1,8-naphthyridin-2-yl)butyl)amino)-2-(pyrimidin-4-ylamino)butanoic acid;
4-((2-fluoro-3-methoxypropyl)(4-(5,6,7,8-tetrahydro-1,8-naphthyridin-2-yl)butyl)amino)-2-((6-phenylpyrimidin-4-yl)amino)butanoic acid;
4-((oxetan-2-ylmethyl)(4-(5,6,7,8-tetrahydro-1,8-naphthyridin-2-yl)butyl)amino)-2-(quinazolin-4-ylamino)butanoic acid;
4-((3-hydroxy-2-(hydroxymethyl)propyl)(4-(5,6,7,8-tetrahydro-1,8-naphthyridin-2-yl)butyl)amino)-2-(quinazolin-4-ylamino)butanoic acid;
2-((5-bromopyrimidin-2-yl)amino)-4-((3,3-difluoropropyl)(4-(5,6,7,8-tetrahydro-1,8-naphthyridin-2-yl)butyl)amino)butanoic acid;
4-((3,3-difluoropropyl)(4-(5,6,7,8-tetrahydro-1,8-naphthyridin-2-yl)butyl)amino)-2-((5-(trifluoromethyl)pyrimidin-2-yl)amino)butanoic acid;
4-((3,3-difluoropropyl)(4-(5,6,7,8-tetrahydro-1,8-naphthyridin-2-yl)butyl)amino)-2-((1-methyl-1H-pyrazolo[3,4-d]pyrimidin-4-yl)amino)butanoic acid;
4-((3,3-difluoropropyl)(4-(5,6,7,8-tetrahydro-1,8-naphthyridin-2-yl)butyl)amino)-2-((2-(trifluoromethyl)pyrimidin-4-yl)amino)butanoic acid;
2-((5-cyclopropylpyrimidin-2-yl)amino)-4-((3,3-difluoropropyl)(4-(5,6,7,8-tetrahydro-1,8-naphthyridin-2-yl)butyl)amino)butanoic acid;
4-((3-fluoropropyl)(4-(5,6,7,8-tetrahydro-1,8-naphthyridin-2-yl)butyl)amino)-2-((1-methyl-1H-pyrazolo[3,4-d]pyrimidin-4-yl)amino)butanoic acid;
4-((3-fluoropropyl)(4-(5,6,7,8-tetrahydro-1,8-naphthyridin-2-yl)butyl)amino)-2-((5-(trifluoromethyl)pyrimidin-2-yl)amino)butanoic acid;
2-((5-cyanopyrimidin-2-yl)amino)-4-((2-(4-fluorophenoxy)ethyl)(4-(5,6,7,8-tetrahydro-1,8-naphthyridin-2-yl)butyl)amino)butanoic acid;
4-((2-(4-fluorophenoxy)ethyl)(4-(5,6,7,8-tetrahydro-1,8-naphthyridin-2-yl)butyl)amino)-2-((5-(trifluoromethyl)pyrimidin-2-yl)amino)butanoic acid;
4-((2-(dimethylamino)-2-oxoethyl)(4-(5,6,7,8-tetrahydro-1,8-naphthyridin-2-yl)butyl)amino)-2-((1-methyl-1H-pyrazolo[3,4-d]pyrimidin-4-yl)amino)butanoic acid;
4-((2-(dimethylamino)-2-oxoethyl)(4-(5,6,7,8-tetrahydro-1,8-naphthyridin-2-yl)butyl)amino)-2-((5-(trifluoromethyl)pyrimidin-2-yl)amino)butanoic acid;
4-((2,2-difluoroethyl)(4-(5,6,7,8-tetrahydro-1,8-naphthyridin-2-yl)butyl)amino)-2-((6-phenylpyrimidin-4-yl)amino)butanoic acid;
2-((1H-pyrazolo[3,4-d]pyrimidin-4-yl)amino)-4-((2-(4-fluorophenoxy)ethyl)(4-(5,6,7,8-tetrahydro-1,8-naphthyridin-2-yl)butyl)amino)butanoic acid;
2-((5-bromopyrimidin-2-yl)amino)-4-((2-(4-fluorophenoxy)ethyl)(4-(5,6,7,8-tetrahydro-1,8-naphthyridin-2-yl)butyl)amino)butanoic acid;
4-((2-(dimethylamino)-2-oxoethyl)(4-(5,6,7,8-tetrahydro-1,8-naphthyridin-2-yl)butyl)amino)-2-((2-(trifluoromethyl)pyrimidin-4-yl)amino)butanoic acid;
2-((5-cyclopropylpyrimidin-2-yl)amino)-4-((2,2-difluoroethyl)(4-(5,6,7,8-tetrahydro-1,8-naphthyridin-2-yl)butyl)amino)butanoic acid; and
4-(((3-fluorooxetan-3-yl)methyl)(4-(5,6,7,8-tetrahydro-1,8-naphthyridin-2-yl)butyl)amino)-2-(quinazolin-4-ylamino)butanoic acid.

In some embodiments, a composition, such as a pharmaceutical composition, is provided wherein the composition comprises a compound selected from the group consisting of one or more of Compound Nos. 1-66 in FIG. 1, or a stereoisomer thereof (including a mixture of two or more stereoisomers thereof), or a salt thereof. In some embodiments, the composition comprises a compound selected from the group consisting of a salt of one or more of Compound Nos. 1-66. In one aspect, the composition is a pharmaceutical composition that further comprises a pharmaceutically acceptable carrier.

In some embodiments, a composition, such as a pharmaceutical composition, is provided wherein the composition comprises a compound selected from the group consisting of one or more of Compound Nos. 1-147, or a stereoisomer thereof (including a mixture of two or more stereoisomers thereof), or a salt thereof. In some embodiments, the composition comprises a compound selected from the group consisting of a salt of one or more of Compound Nos. 1-147. In one aspect, the composition is a pharmaceutical composition that further comprises a pharmaceutically acceptable carrier.

In some embodiments, a composition, such as a pharmaceutical composition, is provided wherein the composition comprises a compound selected from the group consisting of one or more of Compound Nos. 1-665, or a stereoisomer thereof (including a mixture of two or more stereoisomers thereof), or a salt thereof. In some embodiments, the composition comprises a compound selected from the group consisting of a salt of one or more of Compound Nos. 1-665. In one aspect, the composition is a pharmaceutical composition that further comprises a pharmaceutically acceptable carrier.

In some embodiments, a composition, such as a pharmaceutical composition, is provided wherein the composition comprises a compound selected from the group consisting of one or more of Compound Nos. 1-780, or a stereoisomer thereof (including a mixture of two or more stereoisomers thereof), or a salt thereof. In some embodiments, the composition comprises a compound selected from the group consisting of a salt of one or more of Compound Nos. 1-780. In one aspect, the composition is a pharmaceutical composition that further comprises a pharmaceutically acceptable carrier.

The invention also includes all salts of compounds referred to herein, such as pharmaceutically acceptable salts. The invention also includes any or all of the stereochemical forms, including any enantiomeric or diastereomeric forms, and any tautomers or other forms of the compounds described. Unless stereochemistry is explicitly indicated in a chemical structure or name, the structure or name is intended to embrace all possible stereoisomers of a compound depicted. In addition, where a specific stereochemical form is depicted, it is understood that other stereochemical forms are also described and embraced by the invention. All forms of the compounds are also embraced by the invention, such as crystalline or non-crystalline forms of the compounds. It is also understood that prodrugs, solvates and metabolites of the compounds are embraced by this disclosure. Compositions comprising a compound of the invention are also intended, such as a composition of substantially pure compound, including a specific stereochemical form thereof. Compositions comprising a mixture of compounds of the invention in any ratio are also embraced by the invention, including mixtures of two or more stereochemical forms of a compound of the invention in any ratio, such that racemic, non-racemic, enantioenriched and scalemic mixtures of a compound are embraced. Where one or more tertiary amine moiety is present in the compound, the N-oxides are also provided and described.

Compounds described herein are α_{V}β₆ integrin inhibitors. In some instances, it is desirable for the compound to inhibit other integrins in addition to α_{V}β₆ integrin. In some embodiments, the compound inhibits α_{V}β₆ integrin and one or more of α_{V}β₁, α_{V}β₃, α_{V}β₅, α_{V}β₁, α₃β₁, α₆β₁, α₇β₁ and α₁₁β₁ integrin. In some embodiments, the compound inhibits α_{V}β₆ integrin and α_{V}β₁ integrin. In some embodiments, the compound inhibits α_{V}β₆ integrin, α_{V}β₃ integrin and a_{V}β₅ integrin. In some embodiments, the compound inhibits α_{V}β₆ integrin and α₂β₁ integrin. In some embodiments, the compound inhibits α_{V}β₆ integrin, α₂β₁ integrin and α₃β₁ integrin. In some embodiments, the compound inhibits α_{V}β₆ integrin and α₆β₁ integrin. In some embodiments, the compound inhibits α_{V}β₆ integrin and a₇β₁ integrin. In some embodiments, the compound inhibits α_{V}β₆ integrin and α₁₁β₁ integrin.

In some instances, it is desirable to avoid inhibition of other integrins. In some embodiments, the compound is a selective α_{V}β₆ integrin inhibitor. In some embodiments, the compound does not inhibit substantially α₄β₁, α_{V}β₈ and/or α₂β₃ integrin. In some embodiments, the compound inhibits α_{V}β₆ integrin but does not inhibit substantially α₄β₁ integrin. In some embodiments, the compound inhibits α_{V}β₆ integrin but does not inhibit substantially α_{V}β₈ integrin. In some embodiments, the compound inhibits α_{V}β₆ integrin but does not inhibit substantially α₂β₃ integrin. In some embodiments, the compound inhibits α_{V}β₆ integrin but does not inhibit substantially the α_{V}β₈ integrin and the α₄β₁ integrin.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Incorporation of heavier isotopes such as deuterium (²H or D) can afford certain therapeutic advantages resulting from greater metabolic stability, for example, increased in vivo half-life, or reduced dosage requirements and, hence may be preferred in some instances. As used herein, each instance of replacement of a hydrogen by deuterium is also a disclosure of replacing that hydrogen with tritium. As used herein, each instance of enrichment, substitution, or replacement of an atom with corresponding isotope of that atom encompasses isotopic enrichment levels of one of about: 50%, 60%, 70%, 80%, 90%, 95%, 96%, 97%, 98%, 99%, 99.5%, 99.6%, 99.7%, 99.8%, 99.9%, or 100%, or a range between any two of the preceding percentages.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

In various embodiments, for each of the compounds named or depicted herein, specifically disclosed are corresponding isotopically substituted compounds according to the following description. For example, disclosed are corresponding isotopically substituted compounds in which the groups corresponding to structural variables R¹ and R^{1a} may be independently deuterated, e.g., structural variables R¹ and R^{1a} may be perdeuterated such that every hydrogen therein may be independently replaced with deuterium. Further disclosed are corresponding isotopically substituted compounds in which one or more hydrogens in the group corresponding to structural variable R¹, but not in optional substituent R^{1a}, may be independently replaced with deuterium. For example, disclosed are corresponding isotopically substituted compounds in which every hydrogen bonded to a ring in the group corresponding to R¹, but not in optional substituent R^{1a}, may be replaced with deuterium. Also disclosed are corresponding isotopically substituted compounds in which one or more hydrogens in R^{1a} may be independently replaced with deuterium, e.g., every hydrogen in the group corresponding to R^{1a} may be replaced with deuterium.

Further disclosed, for example, are corresponding isotopically substituted compounds in which the groups corresponding to structural variables R² and R^{2a} may be independently deuterated, e.g., structural variables R² and R^{2a} may be perdeuterated such that every hydrogen therein may be independently replaced with deuterium. Also disclosed are corresponding isotopically substituted compounds in which one or more hydrogens in the group corresponding to R², but not in optional substituent R^{2a}, may be independently replaced with deuterium. Additionally disclosed are corresponding isotopically substituted compounds in which each hydrogen at the 1-position of R², the carbon bonding R² to the rest of the compound, may be independently replaced with deuterium. For example, for named compounds having —CH2CH2CH2F corresponding to R², also disclosed are corresponding isotopically substituted compounds in which R² is —CD₂CH₂CH₂F; for named compounds having -CH₂-cyclopropyl corresponding to R², also disclosed are corresponding isotopically substituted compounds in which R² is -CD₂-cyclopropyl; and the like. Disclosed are corresponding isotopically substituted compounds in which each hydrogen in the group corresponding to R^{2a} may be independently replaced with deuterium. For example, for each compound in which R^{2a} is —OCH₃, also disclosed are corresponding isotopically substituted compounds in which R^{2a} may be —OCD₃; for each compound in which R^{2a} is —N(CH₃)₂, also disclosed are corresponding isotopically substituted compounds in which R^{2a} may be —N(CD₃)₂; and the like. Further disclosed are compounds in which the 1-position of R² may be di-deuterated and each hydrogen in the group corresponding to R^{2a} may be replaced with deuterium.

Also disclosed are corresponding isotopically substituted compounds in which R¹⁰, R¹¹, R¹², R¹³, and each R¹⁴ are independently deuterated. For example, disclosed are corresponding isotopically substituted compounds in which R¹⁰, R¹¹ are deuterium, or R¹², R¹³ are deuterium, or R¹⁰, R¹¹, R¹², and R¹³ are all deuterium. Further disclosed are compounds in which R¹⁴ is deuterium and R¹⁴ substitutes the tetrahydronaphthyridine-2-yl group at the 3-position, the 4-position, or the 3- and 4-positions. Also disclosed are compounds in which R¹⁴ is deuterium and each R¹⁴ independently replaces each hydrogen in the tetrahydronaphthyridine-2-yl group at the 5-position, the 6-position, the 7-position, the 5-and 6-positions, the 5- and 7-positions, the 6- and 7-positions, or the 5-, 6-, and 7-positions, e.g., the 7-position may be substituted with two deuterium atoms.

In some embodiments, disclosed are corresponding isotopically substituted compounds in which: every ring hydrogen in R¹ may be replaced with deuterium; the 1-position of R² may be di-deuterated; and R^{2a} may be perdeuterated. Disclosed are corresponding isotopically substituted compounds in which every ring hydrogen in R¹ may be replaced with deuterium. Disclosed are corresponding isotopically substituted compounds in which: every ring hydrogen in R¹ may be replaced with deuterium; the 1-position of R² may be di-deuterated; R^{2a} may be perdeuterated; R¹² and R¹³ may be deuterium; and the 7-position of the tetrahydronaphthyridine-2-yl group may be di-deuterated. Disclosed are corresponding isotopically substituted compounds in which: every ring hydrogen in R¹ may be replaced with deuterium; and each hydrogen in R^{2a} may be independently replaced with deuterium. Disclosed are corresponding isotopically substituted compounds in which: every ring hydrogen in R¹ may be replaced with deuterium; the 1-position of R² may be di-deuterated; R^{2a} may be perdeuterated; and R¹² and R¹³ may be deuterium. Disclosed are corresponding isotopically substituted compounds in which: R¹ and R^{1a} may be perdeuterated; the 1-position of R² may be di-deuterated; R^{2a} may be perdeuterated; R¹² and R¹³ may be deuterium; and the 7-position of the tetrahydronaphthyridine-2-yl group may be di-deuterated. Disclosed are corresponding isotopically substituted compounds in which: every ring hydrogen in R¹ may be replaced with deuterium; the 1-position of R² may be di-deuterated; R^{2a} may be perdeuterated; and R¹² and R¹³ may be deuterium.

In some embodiments of the named compounds, each hydrogen represented in R¹, R^{1a}, R², R^{2a}, R¹⁰, R¹¹, R¹², R¹³, and R¹⁴ may independently be tritium. For example, disclosed are corresponding isotopically substituted compounds in which one or more hydrogens in R¹, R^{1a}, or R¹ and R^{1a} may be independently be replaced by tritium. Disclosed are corresponding isotopically substituted compounds in which one or more ring hydrogens in R¹, R^{1a}, or R¹ and R^{1a} may be independently be replaced by tritium. Disclosed are corresponding isotopically substituted compounds in which one or more hydrogens in R², R^{2a}, or R² and R^{2a} may be independently be replaced by tritium. Disclosed are corresponding isotopically substituted compounds in which one or more hydrogens in R², R^{2a}, or R² and R^{2a} may be independently be replaced by tritium. Disclosed are corresponding isotopically substituted compounds in which one of the 3- or 4-positions of the tetrahydronaphthyridine-2-yl group may be tritiated, e.g., the 3-position. Disclosed are corresponding isotopically substituted compounds in which one of the 5-, 6-, or 7-positions of the tetrahydronaphthyridine-2-yl group may be mono- or di-tritiated, e.g., the 7-position may be di-tritiated.

In some embodiments of the named compounds, disclosed are corresponding isotopically substituted compounds in which one or more carbons may be replaced with ¹³C. For example, disclosed are corresponding isotopically substituted compounds in which one or more carbons may be replaced with ¹³C, such as carbons in R¹, R^{1a}, R², R^{2a}, the tetrahydronaphthyridine-2-yl ring depicted in the structural formulas herein, and the like. For example, in rings represented by R¹, R^{1a}, R², R^{2a}, and/or the tetrahydronaphthyridine-2-yl group, one or more ring carbons may be replaced with ¹³C. For example, polycyclic rings represented by R¹, R^{1a}, R², R^{2a}, and/or the tetrahydronaphthyridine-2-yl group, one or more ring carbons in the ring directly bonded to the rest of the compound may be replaced with ¹³C; e.g., in the tetrahydronaphthyridine-2-yl group, the ring directly bonded to the rest of the compound is a heteroaromatic ring bonded at the 2-position. In polycyclic rings in the groups corresponding to R¹, R^{1a}, R², R^{2a}, and/or the tetrahydronaphthyridine-2-yl group, one or more ring carbons may be replaced with ¹³C in a ring that substitutes or is fused to the ring bonded to the rest of the compound. For example, in the tetrahydronaphthyridine-2-yl ring, the nonaromatic heterocyclyl ring is fused to the ring bonded to the rest of the compound. Further, for example, every ring carbon, or every carbon in the group corresponding to R¹, R^{1a}, R², R^{2a}, and/or the tetrahydronaphthyridine-2-yl ring may be replaced with ¹³C.

The invention also includes any or all metabolites of any of the compounds described. The metabolites may include any chemical species generated by a biotransformation of any of the compounds described, such as intermediates and products of metabolism of the compound.

Articles of manufacture comprising a compound of the invention, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms.

### General Synthetic Methods

The compounds of the invention may be prepared by a number of processes as generally described below and more specifically in the Examples hereinafter (such as the schemes provides in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization, and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a pharmaceutically acceptable salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

Compounds provided herein may be prepared according to General Schemes A, B, C, and D, General Procedures A, B, C, D, E, F, G, H, and P, and the examples herein.

Compounds provided herein may be prepared according to General Schemes A, B, C, and D, General Procedures A, B, C, D, E, F, G, H, P, Q, R, S, T, and U, and the examples herein.

Compounds of formula 11A can be prepared according to General Scheme A, wherein R¹ and R² are as defined for formula (I), or any applicable variations detailed herein.

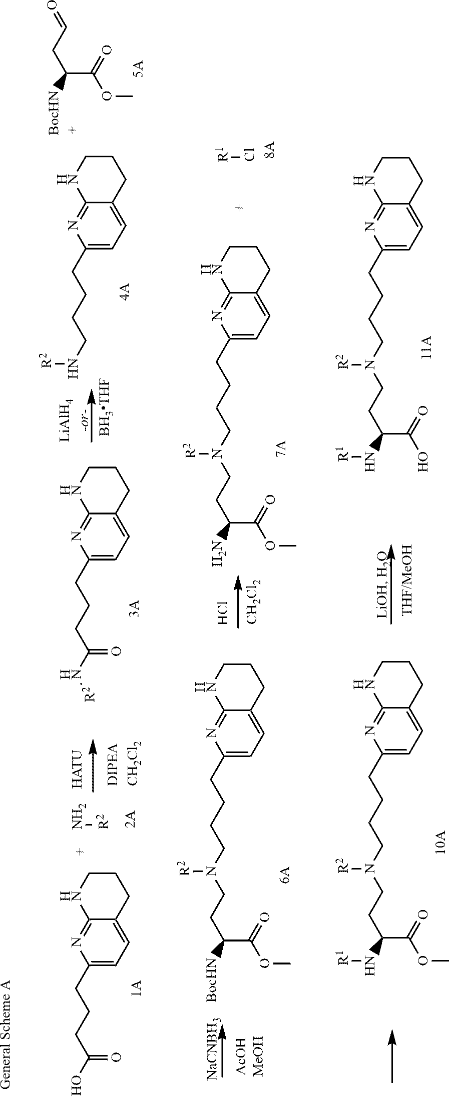

Coupling of 1A with a compound of formula 2A in the presence of a suitable coupling agent yields a compound of formula 3A, which is reduced to yield a compound of formula 4A. Reductive amination of a compound of formula 4A with compound 5A gives a compound of formula 6A. Removal of the N-Boc protecting group with a compound of formula 6A by exposure to an appropriate acid gives a compound of formula 7A, which can be coupled with a compound of formula 8A to give a compound of formula 10A. Hydrolysis of a compound of formula 10A in the presence of a suitable hydroxide source gives compounds of formula 11A.

Reaction conditions for the transformations of General Scheme A are provided in the General Procedures that follow, in particular General Procedures A, D, E, F, G, H, and P.

General Scheme A can be modified to prepare variants of compounds of formula 11A by beginning with variants of 1A with 5 and 6 carbon linkers between the nitrogen bearing the R² group and the tetrahydronaphthyridine group. These variants of compounds of formula 11A can be synthesized by using the route described in General Scheme A substituting 1A with either 5,6,7,8-tetrahydro-1,8-naphthyridine-2-pentanoic acid or 5,6,7,8-tetrahydro-1,8-naphthyridine-2-hexanoic acid. 6-oxoheptanoic acid and 7-oxooctanoic acid can be converted to 5,6,7,8-tetrahydro-1,8-naphthyridine-2-pentanoic acid and 5,6,7,8-tetrahydro-1,8-naphthyridine-2-hexanoic acid, respectively, by condensation with 2-aminonicotinaldehyde in the presence of an appropriate catalyst followed by hydrogenation of the resulting naphthyridine ring to the 5,6,7,8-tetrahydronaphthyridine ring using procedures known in the chemical literature.

Compounds of formula 11A can alternatively be prepared according to General Scheme B, wherein R¹ and R² are as defined for formula (I), or any applicable variations detailed herein.

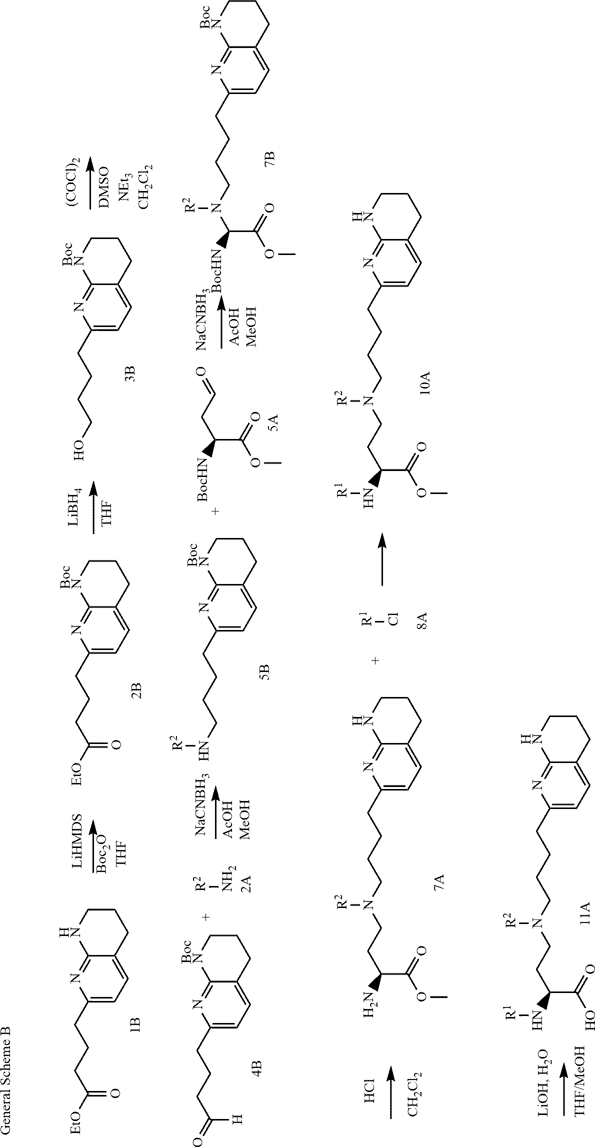

Installation of a N-Boc group of 1B in the presence of a suitable base and di-tert-butyl decarbonate yields a compound of formula 2B, which is reduced to yield a compound of formula 3B. Oxidation of a compound of formula 3B with a suitable oxidizing agent gives a compound of formula 4B. Reductive amination of a compound of formula 4B with compound 2A gives a compound of formula 5B. Reductive amination of a compound of formula 5B with compound 5A gives a compound of formula 7B. Removal of the N-Boc protecting group with a compound of formula 7B by exposure to an appropriate acid gives a compound of formula 7A, which can be coupled with a compound of formula 8A to give a compound of formula 10A. Hydrolysis of a compound of formula 10A in the presence of a suitable hydroxide source gives compounds of formula 11A.

Reaction conditions for the transformations of General Scheme B are provided in the General Procedures that follow, in particular General Procedures B, D, F, G, H, and P.

General Scheme B can be modified to prepare variants of compounds of formula 11A by beginning with variants of 1B with 5 and 6 carbon linkers between the nitrogen bearing the R² group and the tetrahydronaphthyridine group. These variants of compounds of formula 11A can be synthesized by using the route described in General Scheme B substituting 1B with either ethyl 5-(5,6,7,8-tetrahydro-1,8-naphthyridin-2-yl)pentanoate or ethyl 6-(5,6,7,8-tetrahydro-1,8-naphthyridin-2-yl)hexanoate. Ethyl 6-oxoheptanoate and ethyl 7-oxooctanoate can be converted to ethyl 5-(5,6,7,8-tetrahydro-1,8-naphthyridin-2-yl)pentanoate and ethyl 6-(5,6,7,8-tetrahydro-1,8-naphthyridin-2-yl)hexanoate, respectively, by condensation with 2-aminonicotinaldehyde in the presence of an appropriate catalyst followed by hydrogenation of the resulting naphthyridine ring to the 5,6,7,8-tetrahydronaphthyridine ring using procedures known in the chemical literature.

Compounds of formula 10C can be prepared according to General Scheme C, wherein R is C₁-C₅ alkyl optionally substituted by R^{2a}, and R¹ and R^{2a} are as defined for formula (I), or any applicable variations detailed herein.

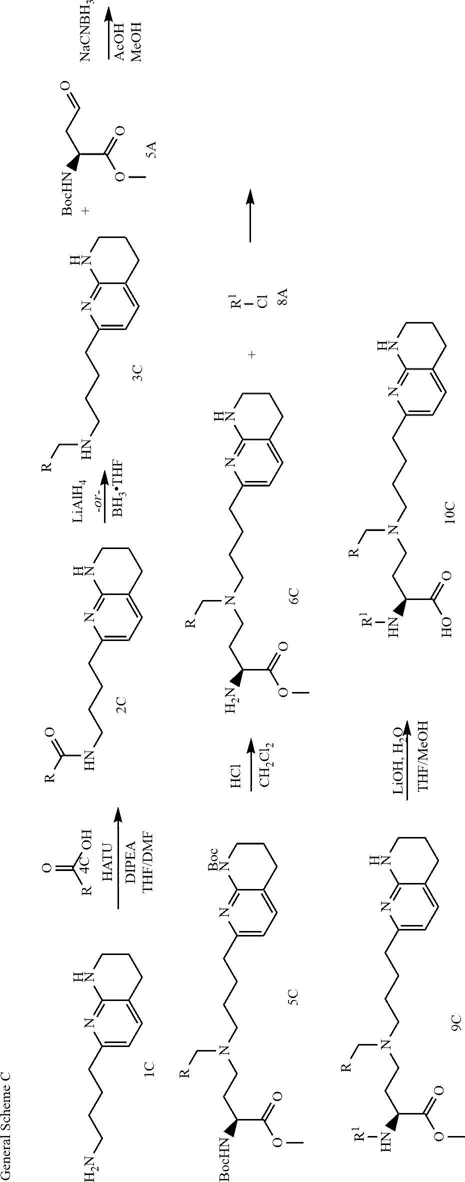

Coupling of 1C with a compound of formula 4C in the presence of a suitable coupling agent yields a compound of formula 2C, which is reduced to yield a compound of formula 3C. Reductive amination of a compound of formula 3C with compound 5A gives a compound of formula 5C. Global removal of the N-Boc protecting groups with a compound of formula 5C by exposure to an appropriate acid gives a compound of formula 6C, which can be coupled with a compound of formula 8A to give a compound of formula 9C. Hydrolysis of a compound of formula 9C in the presence of a suitable hydroxide source gives compounds of formula 10C.

Reaction conditions for the transformations of General Scheme C are provided in the General Procedures that follow, in particular General Procedures B, D, F, G, H, and P.

General Scheme C can be modified to prepare variants of compounds of formula 10C by beginning with variants of 1C with 5 and 6 carbon linkers between the nitrogen bearing the -CH₂R group and the tetrahydronaphthyridine group. These variants of compounds of formula 10C can be synthesized by using the route described in General Scheme C substituting 1C with either 5-(5,6,7,8-tetrahydro-1,8-naphthyridin-2-yl)pentan-1-amine or 6-(5,6,7,8-tetrahydro-1,8-naphthyridin-2-yl)hexan-1-amine. 6-oxoheptanoic acid and 7-oxooctanoic acid can be converted to 5,6,7,8-tetrahydro-1,8-naphthyridine-2-pentanoic acid and 5,6,7,8-tetrahydro-1,8-naphthyridine-2-hexanoic acid, respectively, by condensation with 2-aminonicotinaldehyde in the presence of an appropriate catalyst followed by hydrogenation of the resulting naphthyridine ring to the 5,6,7,8-tetrahydronaphthyridine ring using procedures known in the chemical literature. The resulting carboxylic acids can be converted to a primary amine by a two-step procedure that includes coupling of the carboxylic acid with an appropriate ammonia source in the presence of suitable coupling reagents followed by reduction.

Compounds of formula 10C can alternatively be prepared according to General Scheme D, wherein R is C₁-C₅ alkyl optionally substituted by R^{2a}, and R¹ and R^{2a} are as defined for formula (I), or any applicable variations detailed herein.

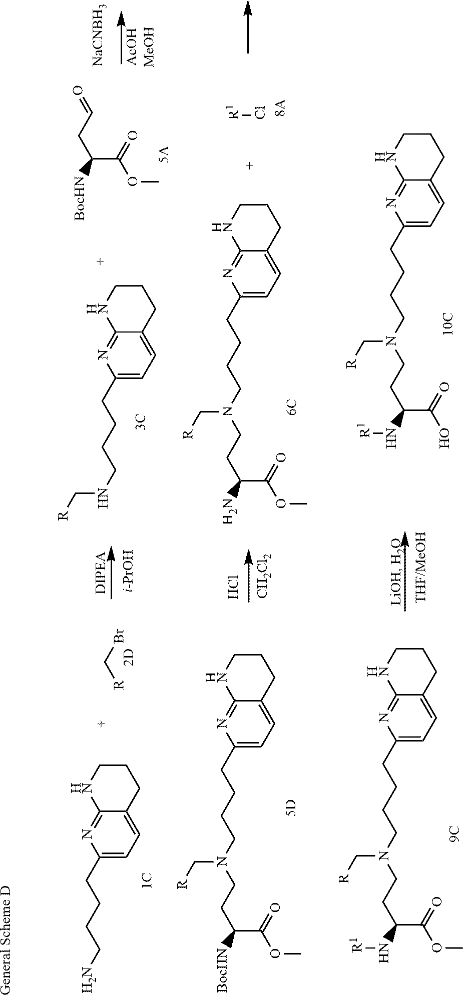

Alkylation of 1C with a compound of formula 2D in the presence of a suitable alkyl halide yields a compound of formula 3C. Reductive amination of a compound of formula 3C with compound 5A gives a compound of formula 5C. Removal of the N-Boc protecting group with a compound of formula 5C by exposure to an appropriate acid gives a compound of formula 6C, which can be coupled with a compound of formula 9A to give a compound of formula 9C. Hydrolysis of a compound of formula 8A in the presence of a suitable hydroxide source gives compounds of formula 10C.

Reaction conditions for the transformations of General Scheme D are provided in the General Procedures that follow, in particular General Procedures C, F, G, H, and P.

General Scheme D can be modified to prepare variants of compounds of formula 10C by beginning with variants of 1C with 5 and 6 carbon linkers between the nitrogen bearing the -CH₂R group and the tetrahydronaphthyridine group. These variants of compounds of formula 10C can be synthesized by using the route described in General Scheme D substituting 1C with either 5-(5,6,7,8-tetrahydro-1,8-naphthyridin-2-yl)pentan-1-amine or 6-(5,6,7,8-tetrahydro-1,8-naphthyridin-2-yl)hexan-1-amine. 6-oxoheptanoic acid and 7-oxooctanoic acid can be converted to 5,6,7,8-tetrahydro-1,8-naphthyridine-2-pentanoic acid and 5,6,7,8-tetrahydro-1,8-naphthyridine-2-hexanoic acid, respectively, by condensation with 2-aminonicotinaldehyde in the presence of an appropriate catalyst followed by hydrogenation of the resulting naphthyridine ring to the 5,6,7,8-tetrahydronaphthyridine ring using procedures known in the chemical literature. The resulting carboxylic acids can be converted to a primary amine by a two-step procedure that includes coupling of the carboxylic acid with an appropriate ammonia source in the presence of suitable coupling reagents followed by reduction.

Compounds of formula 1f can be prepared according to General Scheme E. It is understood the ring bearing the Het description can be any heteroaromatic ring.

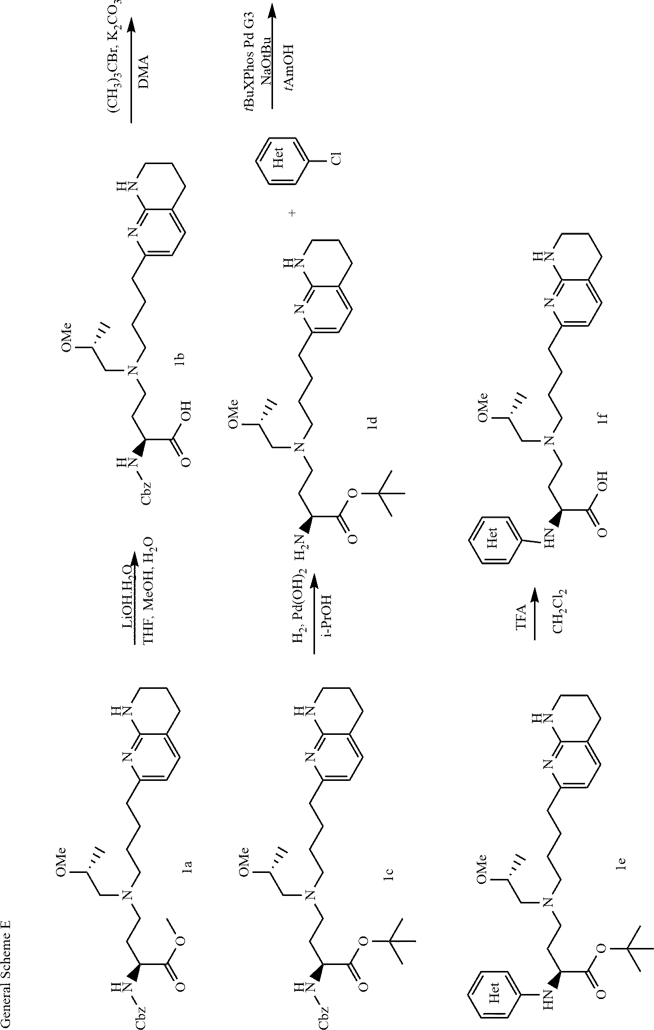

Hydrolysis of a compound of formula 1a gives a compound of formula 1b which can be alkylated with a suitable electrophile to give a compound of formula 1c. Deprotection under reductive conditions of a compound of formula 1c gives a compound of formula 1d. Metal catalyzed cross coupling of a halogenated arene with a compound of formula 1d gives a compound of formula 1e, which can be hydrolyzed under acidic conditions to give compound of formula 1f.

Reaction conditions for the transformations of General Scheme E are provided in the General Procedures that follow, in particular General Procedures Q, R, S, T, and U.

It is understood that the schemes above may be modified to arrive at various compounds of the invention by selection of appropriate reagents and starting materials. For a general description of protecting groups and their use, see P.G.M. Wuts and T.W. Greene, Greene’s Protective Groups in Organic Synthesis 4^{th} edition, Wiley-Interscience, New York, 2006.

Additional methods of preparing compounds according to Formula (I), and salts thereof, are provided in the Examples. As a skilled artisan would recognize, the methods of preparation taught herein may be adapted to provide additional compounds within the scope of Formula (I), for example, by selecting starting materials which would provide a desired compound.

### Pharmaceutical Compositions and Formulations

Pharmaceutical compositions of any of the compounds detailed herein, including compounds of the formula (I), (I-A), (I-B), (I-C), (I-D), (I-E), (I-F), (I-G), (I-H), (II), (II-A), (II-B), (II-C), (II-D), (II-E), (II-F), (II-G), or (II-H), or a salt thereof, or any of compounds of FIG. 1, or a salt thereof, or mixtures thereof, are embraced by this invention. Pharmaceutical compositions of any of the compounds detailed herein, including compounds of the formula (I), (I-A), (I-B), (I-C), (I-D), (I-E), (I-F), (I-G), (I-H), (II), (II-A), (II-B), (II-C), (II-D), (II-E), (II-F), (II-G), or (II-H), or a salt thereof, or any of compounds of FIG. 1, or a salt thereof, or mixtures thereof, are embraced by this invention. Pharmaceutical compositions of compounds of the formula (A), or a salt thereof, or mixtures thereof, are embraced by this invention. Thus, the invention includes pharmaceutical compositions comprising a compound of the invention or a pharmaceutically acceptable salt thereof and a pharmaceutically acceptable carrier or excipient. In one aspect, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation. In one embodiment, the pharmaceutical composition is a composition for controlled release of any of the compounds detailed herein.

A compound as detailed herein may in one aspect be in a purified form and compositions comprising a compound in purified forms are detailed herein. In one embodiment, compositions may have no more than 35% impurity, wherein the impurity denotes a compound other than the compound comprising the majority of the composition or a salt thereof, for example, a composition of a compound selected from a compound of FIG. 1 may contains no more than 35% impurity, wherein the impurity denotes a compound other than the compound of FIG. 1 or a salt thereof. In one embodiment, compositions may have no more than 35% impurity, wherein the impurity denotes a compound other than the compound comprising the majority of the composition or a salt thereof, for example, a composition of a compound selected from a compound of FIG. 1 may contain no more than 35% impurity, wherein the impurity denotes a compound other than the compound of FIG. 1, or a salt thereof. In one embodiment, compositions may contain no more than 25% impurity. In one embodiment, compositions may contains no more than 20% impurity. In still further embodiments, compositions comprising a compound as detailed herein or a salt thereof are provided as compositions of substantially pure compounds. “Substantially pure” compositions comprise no more than 10% impurity, such as a composition comprising less than 9%, 7%, 5%, 3%, 1%, or 0.5% impurity. In some embodiments, a composition containing a compound as detailed herein or a salt thereof is in substantially pure form. In still another variation, a composition of substantially pure compound or a salt thereof is provided wherein the composition contains or no more than 10% impurity. In a further variation, a composition of substantially pure compound or a salt thereof is provided wherein the composition contains or no more than 9% impurity. In a further variation, a composition of substantially pure compound or a salt thereof is provided wherein the composition contains or no more than 7% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 10% or preferably no more than 5% or more preferably no more than 3% or even more preferably no more than 1% impurity or most preferably no more than 0.5% impurity, which impurity may be the compound in a different stereochemical form. For instance, a composition of substantially pure (S) compound means that the composition contains no more than 10% or no more than 5% or no more than 3% or no more than 1% or no more than 0.5% of the (R) form of the compound.

In one variation, the compounds herein are synthetic compounds prepared for administration to an individual such as a human. In another variation, compositions are provided containing a compound in substantially pure form. In another variation, the invention embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

In one embodiment, the compounds can be administered in the liquid vehicle ORA-SWEET® from PERRIGO®, Allegan, Michigan, which is a syrup vehicle having ingredients of purified water, glycerin, sorbitol, sodium saccharin, xanthan gum, and flavoring, buffered with citric acid and sodium citrate, preserved with methylparaben (0.03%), potassium sorbate (0.1%), and propylparaben (0.008%); or in a mixture of ORA-SWEET® and water of any proportion, such as a 50:50 mixture of ORA-SWEET® to water. The water used should be a pharmaceutically acceptable grade of water, for example, sterile water.

Any of the compounds described herein can be formulated in a tablet in any dosage form described, for example, a compound as described herein or a pharmaceutically acceptable salt thereof can be formulated as a 10 mg tablet.

Compositions comprising a compound provided herein are also described. In one variation, the composition comprises a compound and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

### Methods of Use

Compounds and compositions of the invention, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided is a method of treating a fibrotic disease in an individual in need thereof comprising administering to the individual a therapeutically effective amount of a compound of formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-66 in FIG. 1, or a stereoisomer thereof, or a pharmaceutically acceptable salt thereof. In one aspect, provided is a method of treating a fibrotic disease in an individual in need thereof comprising administering to the individual a therapeutically effective amount of a compound of formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-147, or a stereoisomer thereof, or a pharmaceutically acceptable salt thereof. In one aspect, provided is a method of treating a fibrotic disease in an individual in need thereof comprising administering to the individual a therapeutically effective amount of a compound of formula (I), or any variation thereof, e.g., a compound of formula (I-A), (I-B), (I-C), (I-D), (I-E), (IF), (I-G), (I-H), (II), (II-A), (II-B), (II-C), (II-D), (II-E), (II-F), (II-G), or (II-H), a compound selected from Compound Nos. 1-665, or a stereoisomer thereof, or a pharmaceutically acceptable salt thereof. In one aspect, provided is a method of treating a fibrotic disease in an individual in need thereof comprising administering to the individual a therapeutically effective amount of a compound of formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof. In one aspect, provided is a method of treating a fibrotic disease in an individual in need thereof comprising administering to the individual a therapeutically effective amount of a compound of formula (A), or any variation thereof, or a stereoisomer thereof, or a pharmaceutically acceptable salt thereof. In one aspect, the individual is a human. The individual, such as human, may be in need of treatment, such as a human who has or is suspected of having a fibrotic disease.

In another aspect, provided is a method of delaying the onset and/or development of a fibrotic disease in an individual (such as a human) who is at risk for developing a fibrotic disease. It is appreciated that delayed development may encompass prevention in the event the individual does not develop the fibrotic disease. An individual at risk of developing a fibrotic disease in one aspect has or is suspected of having one or more risk factors for developing a fibrotic disease. Risk factors for fibrotic disease may include an individual’s age (e.g., middle-age or older adults), the presence of inflammation, having one or more genetic component associated with development of a fibrotic disease, medical history such as treatment with a drug or procedure believed to be associated with an enhanced susceptibility to fibrosis (e.g., radiology) or a medical condition believed to be associated with fibrosis, a history of smoking, the presence of occupational and/or environmental factors such as exposure to pollutants associated with development of a fibrotic disease. In some embodiments, the individual at risk for developing a fibrotic disease is an individual who has or is suspected of having NAFLD, NASH, CKD, scleroderma, Crohn’s Disease, NSIP, PSC, PBC, or is an individual who has had or is suspected of having had a myocardial infarction. In some embodiments, the individual at risk for developing a fibrotic disease has or is suspected of having psoriasis.

In some embodiments, the fibrotic disease is fibrosis of a tissue such as the lung (pulmonary fibrosis), the liver, the skin, the heart (cardiac fibrosis), the kidney (renal fibrosis), or the gastrointestinal tract (gastrointestinal fibrosis).

In some embodiments, the fibrotic disease is pulmonary fibrosis (such as IPF), liver fibrosis, skin fibrosis, scleroderma, cardiac fibrosis, renal fibrosis, gastrointestinal fibrosis, primary sclerosing cholangitis, or biliary fibrosis (such as PBC). In some embodiments, the fibrotic disease is pulmonary fibrosis (such as IPF), liver fibrosis, skin fibrosis, psoriasis, scleroderma, cardiac fibrosis, renal fibrosis, gastrointestinal fibrosis, primary sclerosing cholangitis, or biliary fibrosis (such as PBC). In some embodiments, the fibrotic disease is psoriasis.

In some embodiments, the fibrotic disease is a pulmonary fibrosis, e.g., idiopathic pulmonary fibrosis (IPF). In some embodiments, the pulmonary fibrosis is, e.g., interstitial lung disease, radiation-induced pulmonary fibrosis, or systemic sclerosis associated interstitial lung disease.

In some embodiments, the fibrotic disease is a primary sclerosing cholangitis, or biliary fibrosis. In some embodiments, the fibrotic disease is primary biliary cholangitis (also known as primary biliary cirrhosis) or biliary atresia.

In some embodiments, the fibrotic disease is fibrotic nonspecific interstitial pneumonia (NSIP).

In some embodiments, the fibrotic disease is a liver fibrosis, e.g., infectious liver fibrosis (from pathogens such as HCV, HBV or parasites such as schistosomiasis), NASH, alcoholic steatosis induced liver fibrosis, and cirrhosis. In some embodiments, the liver fibrosis is nonalcoholic fatty liver disease (NAFLD). In some embodiments, the liver fibrosis is NASH.

In some embodiments, the fibrotic disease is biliary tract fibrosis.

In some embodiments, the fibrotic disease is renal fibrosis, e.g., diabetic nephrosclerosis, hypertensive nephrosclerosis, focal segmental glomerulosclerosis (“FSGS”), and acute kidney injury from contrast induced nephropathy. In several embodiments, the fibrotic disease is diabetic nephropathy, diabetic kidney disease, or chronic kidney disease.

In some embodiments, the fibrotic disease is characterized by one or more of glomerulonephritis, end-stage kidney disease, hearing loss, changes to the lens of the eye, hematuria, or proteinuria. In some embodiments, the fibrotic disease is Alport syndrome.

In some embodiments, the fibrotic disease is systemic and local sclerosis or scleroderma, keloids and hypertrophic scars, or post surgical adhesions. In some embodiments, the fibrotic disease is scleroderma or systemic sclerosis.

In some embodiments, the fibrotic disease is atherosclerosis or restenosis.

In some embodiments, the fibrotic disease is a gastrointestinal fibrosis, e.g., Crohn’s disease.

In some embodiments, the fibrotic disease is cardiac fibrosis, e.g., post myocardial infarction induced fibrosis and inherited cardiomyopathy.

In some embodiments, the fibrotic disease is psoriasis.

In some embodiments, methods may include modulating the activity of at least one integrin in a subject in need thereof. For example, the method may include modulating the activity of α_{V}β₆. The method may include modulating the activity of α_{V}β₁. The method may include modulating the activity of α_{V}β₁ and α_{V}β₆. Modulating the activity of the at least one integrin may include, e.g., inhibiting the at least one integrin. The method may include administering to the subject an amount of the compound or a pharmaceutically acceptable salt thereof effective to modulate the activity of the at least one integrin in the subject, e.g., at least one of α_{V}β₁ and α_{V}β₆. The subject in need of modulating the activity of at least one integrin may have any of the fibrotic disease or conditions described herein. For example, the fibrotic disease or condition may include idiopathic pulmonary fibrosis, interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis, primary biliary cholangitis (also known as primary biliary cirrhosis), biliary atresia, systemic sclerosis associated interstitial lung disease, scleroderma (also known as systemic sclerosis), diabetic nephropathy, diabetic kidney disease, focal segmental glomerulosclerosis, chronic kidney disease, or Crohn’s Disease. The fibrotic disease or condition may include psoriasis. The method may include administering to the subject an amount of the compound or a pharmaceutically acceptable salt thereof effective to modulate the activity of the at least one integrin in the subject, e.g., at least one of α_{V}β₁ and α_{V}β₆, the subject being in need of treatment for NASH. The method may include administering to the subject an amount of the compound or a pharmaceutically acceptable salt thereof effective to modulate the activity of the at least one integrin in the subject, e.g., at least one of α_{V}β₁ and α_{V}β₆, the subject being in need of treatment for IPF.

The fibrotic disease may be mediated primarily by α_{V}β₆, for example, the fibrotic disease may include idiopathic pulmonary fibrosis or renal fibrosis. Accordingly, the method may include modulating the activity of α_{V}β₆ to treat conditions primarily mediated by α_{V}β₆ such as IPF. The fibrotic disease may be mediated primarily by α_{V}β₁, for example, the fibrotic disease may include NASH. Accordingly, the method may include modulating the activity of α_{V}β₁ to treat conditions primarily mediated by α_{V}β₁, e.g., NASH. The fibrotic disease may be mediated by α_{V}β₁ and α_{V}β₆, for example, the fibrotic disease may include PSC or biliary atresia. Accordingly, the method may include modulating the activity of α_{V}β₁ and α_{V}β₆ to treat conditions mediated by both α_{V}β₁ and α_{V}β₆.

The compound may be a modulator, e.g., an inhibitor, of α_{V}β₁. The compound may be a modulator, e.g., an inhibitor, of α_{V}β₆. The compound may be a dual modulator, such as a dual inhibitor, e.g., dual selective inhibitor, of α_{V}β₁ and α_{V}β₆. For example, Table B-3 demonstrates that some exemplary compounds primarily inhibit α_{V}β₁ over α_{V}β₆; some exemplary compounds primarily inhibit α_{V}β₆ over α_{V}β₁; and some exemplary compounds inhibit α_{V}β₁ and α_{V}β₆, comparably, and may be considered, e.g., “dual α_{V}β₁/α_{V}β₆ inhibitors.”

Modulating or inhibiting the activity of one or both of α_{V}β₁ integrin and α_{V}β₆ integrin, thereby treating a subject with a fibrotic disease, indicates that α_{V}β₁ integrin, α_{V}β₆ integrin, or α_{V}β₁ integrin and α_{V}β₆ integrin are modulated or inhibited to a degree sufficient to treat the fibrotic disease in the subject.

In one aspect, provided is a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-66 in FIG. 1, or a stereoisomer thereof, or a pharmaceutically acceptable salt thereof, for use in the treatment of a fibrotic disease.

In one aspect, provided is a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-147, or a stereoisomer thereof, or a pharmaceutically acceptable salt thereof, for use in the treatment of a fibrotic disease.

In one aspect, provided is a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-665, or a stereoisomer thereof, or a pharmaceutically acceptable salt thereof, for use in the treatment of a fibrotic disease.

In one aspect, provided is a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof, for use in the treatment of a fibrotic disease.

Also provided is use of a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-66 in FIG. 1, or a stereoisomer thereof, or a pharmaceutically acceptable salt thereof, in the manufacture of a medicament for the treatment of a fibrotic disease.

Also provided is use of a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-147, or a stereoisomer thereof, or a pharmaceutically acceptable salt thereof, in the manufacture of a medicament for the treatment of a fibrotic disease.

Also provided is use of a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-665, or a stereoisomer thereof, or a pharmaceutically acceptable salt thereof, in the manufacture of a medicament for the treatment of a fibrotic disease.

Also provided is use of a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof, in the manufacture of a medicament for the treatment of a fibrotic disease.

In another aspect, provided herein is a method of treating a subject in need thereof, comprising administering to the subject a therapeutically effective amount of a compound of formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-66 in FIG. 1, or a stereoisomer thereof, or a pharmaceutically acceptable salt thereof, or a dosage form disclosed herein, wherein the subject has at least one tissue in need of therapy and the tissue has at least one elevated level of: αvβ₁ integrin activity and/or expression; αvβ₆ integrin activity and/or expression; a pSMAD/SMAD value; new collagen formation or accumulation; total collagen; and Type I Collagen gene Col1a1 expression; and wherein the level is elevated compared to a healthy state of the tissue. In some embodiments, the at least one tissue in the subject comprises one or more of: lung tissue, liver tissue, skin tissue, cardiac tissue, kidney tissue, gastrointestinal tissue, gall bladder tissue, and bile duct tissue. In some embodiments, the tissue has an elevated pSMAD2/SMAD2 value or an elevated pSMAD3/SMAD3 value compared to the healthy state of the tissue.

Methods of determining the values of αvβ₁ integrin activity and/or expression; αvβ₆ integrin activity and/or expression; a pSMAD/SMAD value; new collagen formation or accumulation; total collagen; and Type I Collagen gene Col1a1 expression are known in the art and exemplary methods are disclosed in the Examples, such as antibody assays of tissue samples, such as a biopsy sample.

In some embodiments, the method selectively reduces αvβ₁ integrin activity and/or expression compared to αvβ₆ integrin activity and/or expression in the subject. In some embodiments, the method selectively reduces αvβ₆ integrin activity and/or expression compared to αvβ₁ integrin activity and/or expression in the subject. In some embodiments, the method reduces both αvβ₁ integrin and αvβ₆ integrin activity and/or expression compared to at least one other αv-containing integrin in the subject. In some embodiments, the activity of αvβ₁ integrin in one or more fibroblasts is reduced in the subject. In some embodiments, the activity of αvβ₆ integrin in one or more epithelial cells is reduced in the subject.

In another aspect, provided herein is a method of treating a subject in need thereof, comprising administering to the subject a therapeutically effective amount of a compound of formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-66 in FIG. 1, or a stereoisomer thereof, or a pharmaceutically acceptable salt thereof, or a dosage form disclosed herein, wherein the subject has at least one tissue in need of therapy and the tissue has at least one elevated level of: αvβ₁ integrin activity and/or expression; αvβ₆ integrin activity and/or expression; a pSMAD/SMAD value; new collagen formation or accumulation; total collagen; and Type I Collagen gene Col1a1 expression; and wherein the level is elevated compared to a healthy state of the tissue. In some embodiments, the at least one tissue in the subject comprises one or more of: lung tissue, liver tissue, skin tissue, cardiac tissue, kidney tissue, gastrointestinal tissue, gall bladder tissue, and bile duct tissue. In some embodiments, the tissue has an elevated pSMAD2/SMAD2 value or an elevated pSMAD3/SMAD3 value compared to the healthy state of the tissue.

Methods of determining the values of αvβ₁ integrin activity and/or expression; αvβ₆ integrin activity and/or expression; a pSMAD/SMAD value; new collagen formation or accumulation; total collagen; and Type I Collagen gene Col1a1 expression are known in the art and exemplary methods are disclosed in the Examples, such as antibody assays of tissue samples, such as a biopsy sample.

In some embodiments, the method selectively reduces αvβ₁ integrin activity and/or expression compared to αvβ₆ integrin activity and/or expression in the subject. In some embodiments, the method selectively reduces αvβ₆ integrin activity and/or expression compared to αvβ₁ integrin activity and/or expression in the subject. In some embodiments, the method reduces both αvβ₁ integrin and αvβ₆ integrin activity and/or expression compared to at least one other αv-containing integrin in the subject. In some embodiments, the activity of αvβ₁ integrin in one or more fibroblasts is reduced in the subject. In some embodiments, the activity of αvβ₆ integrin in one or more epithelial cells is reduced in the subject.

Also provided herein is a method of characterizing the antifibrotic activity of a small molecule in a subject, comprising: providing a first live cell sample from the subject, the first live cell sample characterized by the presence of at least one integrin capable of activating transforming growth factor β (TGF-β) from latency associated peptide-TGF-P; determining a first pSMAD/SMAD value in the first live cell sample; administering the small molecule to the subject; providing a second live cell sample from the subject, the second live cell sample being drawn from the same tissue in the subject as the first live cell sample; determining a second pSMAD/SMAD value in the second live cell sample; and characterizing the antifibrotic activity of the small molecule in the subject by comparing the second pSMAD/SMAD value to the first pSMAD/SMAD value. In some embodiments, the small molecule is a compound disclosed herein, optionally in a dosage form disclosed herein.

In some embodiments, each live cell sample is a plurality of cells derived from a tissue of the subject, or a plurality of macrophages associated with the tissue of the subject. In some embodiments, the tissue comprises one of: lung tissue, liver tissue, skin tissue, cardiac tissue, kidney tissue, gastrointestinal tissue, gall bladder tissue, and bile duct tissue. In some embodiments, each live cell sample comprises a plurality of alveolar macrophages derived from a bronchoalveolar lavage fluid of the subject.

In some embodiments, the method further comprising conducting a bronchoalveolar lavage on a lung of the subject effective to produce a bronchoalveolar lavage fluid that comprises the plurality of macrophages as a plurality of alveolar macrophages.

In some embodiments, the subject has a fibrotic disease selected from the group consisting of: idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, scleroderma, diabetic nephropathy, diabetic kidney disease, focal segmental glomerulosclerosis, chronic kidney disease, and Crohn’s Disease. In some embodiments, the subject has the fibrotic disease psoriasis.

In some embodiments, the subject is diagnosed with a fibrotic disease selected from the group consisting of: idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, scleroderma, diabetic nephropathy, diabetic kidney disease, focal segmental glomerulosclerosis, chronic kidney disease, Crohn’s Disease, and psoriasis. In some embodiments, the subject is diagnosed with a fibrotic disease at the age of about 55 years or older, about 60 years or older, about 65 years or older, about 70 years or older, or about 75 years or older, for example, idiopathic pulmonary fibrosis (IPF) or psoriasis.

In some embodiments, the subject has a gender-age-physiology (GAP) stage, based on the gender-age-physiology (GAP) index system, of GAP Stage I. In some embodiments, the subject has a GAP stage of GAP Stage II. In some embodiments, the subject has a GAP stage of GAP Stage III.

In some embodiments, the at least one integrin comprises αv. In some embodiments, the at least one integrin comprises αvβ₁. In some embodiments, the at least one integrin comprises αvβ₆.

In some embodiments, determining the first pSMAD/SMAD value in the at least one live cell comprises determining a pSMAD2/SMAD2 value or a pSMAD3/SMAD3 value; and determining the second pSMAD/SMAD value in the at least one live cell after contacting the at least one live cell with the small molecule comprises determining a pSMAD2/SMAD2 value or a pSMAD3/SMAD3 value.

Also provided herein is a method of treating a fibrotic disease in a subject in need thereof, comprising: providing a first live cell sample from the subject, the first live cell sample having at least one integrin capable of activating transforming growth factor β (TGF-β) from latency associated peptide-TGF-P; determining a first pSMAD/SMAD value in the first live cell sample; administering a small molecule to the subject; providing a second live cell sample from the subject, the second live cell sample being drawn from the same tissue in the subject as the first live cell sample; determining a second pSMAD/SMAD value in the second live cell sample; comparing the second pSMAD/SMAD value to the first pSMAD/SMAD value; and administering the small molecule to the subject if the second pSMAD/SMAD value is lower than the first pSMAD/SMAD value. In some embodiments, the small molecule is a compound disclosed herein or a salt thereof, optionally in a dosage form disclosed herein. In some embodiments, the first live cell sample is obtained from the subject prior to treatment with a small molecule.

In some embodiments, each live cell sample is a plurality of cells derived from a tissue of the subject, or a plurality of macrophages associated with the tissue of the subject. In some embodiments, the tissue comprises one of: lung tissue, liver tissue, skin tissue, cardiac tissue, kidney tissue, gastrointestinal tissue, gall bladder tissue, and bile duct tissue. In some embodiments, each live cell sample comprises a plurality of alveolar macrophages derived from a bronchoalveolar lavage fluid of the subject. In some embodiments, the method further comprising conducting a bronchoalveolar lavage on a lung of the subject effective to produce a bronchoalveolar lavage fluid that comprises the plurality of macrophages as a plurality of alveolar macrophages.

In some embodiments, the subject is characterized by having a fibrotic disease selected from the group consisting of: idiopathic pulmonary fibrosis (IPF), interstitial lung disease, radiation-induced pulmonary fibrosis, nonalcoholic fatty liver disease (NAFLD), nonalcoholic steatohepatitis (NASH), alcoholic liver disease induced fibrosis, Alport syndrome, primary sclerosing cholangitis (PSC), primary biliary cholangitis, biliary atresia, systemic sclerosis associated interstitial lung disease, scleroderma, diabetic nephropathy, diabetic kidney disease, focal segmental glomerulosclerosis, chronic kidney disease, and Crohn’s Disease. In some embodiments, the subject is characterized by having psoriasis.

In some embodiments, the at least one integrin comprises αv. In some embodiments, the at least one integrin comprises αvβ₁. In some embodiments, the at least one integrin comprises αvβ₆.

In some embodiments, determining the first pSMAD/SMAD value in the first live cell sample comprises determining a pSMAD2/SMAD2 value or a pSMAD3/SMAD3 value; and determining the second pSMAD/SMAD value in the at least one live cell after contacting the first live cell sample with the small molecule comprises determining a pSMAD2/SMAD2 value or a pSMAD3/SMAD3 value.

In another aspect, provided is a method of inhibiting αvβ₆ integrin in an individual comprising administering a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a stereoisomer thereof, or a compound selected from Compound Nos. 1-66 in FIG. 1, or a pharmaceutically acceptable salt thereof.

In another aspect, provided is a method of inhibiting αvβ₆ integrin in an individual comprising administering a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a stereoisomer thereof, or a compound selected from Compound Nos. 1-147, or a pharmaceutically acceptable salt thereof.

In another aspect, provided is a method of inhibiting αvβ₆ integrin in an individual comprising administering a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a stereoisomer thereof, or a compound selected from Compound Nos. 1-665, or a pharmaceutically acceptable salt thereof.

In another aspect, provided is a method of inhibiting αvβ₆ integrin in an individual comprising administering a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a stereoisomer thereof, or a compound selected from Compound Nos. 1-780, or a pharmaceutically acceptable salt thereof.

Also provided is a method of inhibiting TGFβ activation in a cell comprising administering to the cell a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-66 in FIG. 1, or a stereoisomer thereof, or a pharmaceutically acceptable salt thereof.

Also provided is a method of inhibiting TGFβ activation in a cell comprising administering to the cell a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-147, or a stereoisomer thereof, or a pharmaceutically acceptable salt thereof.

Also provided is a method of inhibiting TGFβ activation in a cell comprising administering to the cell a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-665, or a stereoisomer thereof, or a pharmaceutically acceptable salt thereof.

Also provided is a method of inhibiting TGFβ activation in a cell comprising administering to the cell a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof.

Also provided is a method of inhibiting αvβ₆ integrin in an individual in need thereof, comprising administering to the individual a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-66 in FIG. 1, or a stereoisomer thereof, or a pharmaceutically acceptable salt thereof. Also provided is a method of inhibiting αvβ₆ integrin in an individual in need thereof, comprising administering to the individual a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-147, or a stereoisomer thereof, or a pharmaceutically acceptable salt thereof. Also provided is a method of inhibiting αvβ₆ integrin in an individual in need thereof, comprising administering to the individual a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-665, or a stereoisomer thereof, or a pharmaceutically acceptable salt thereof. Also provided is a method of inhibiting αvβ₆ integrin in an individual in need thereof, comprising administering to the individual a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof. In one such method, the compound is a selective αvβ₆ integrin inhibitor. In another such method, the compound does not inhibit substantially α₄β₁, αvβ₈ and/or α₂β₃ integrin. In yet another such method, the compound inhibits αvβ₆ integrin but does not inhibit substantially α₄β₁ integrin. In still another such method, the compound inhibits αvβ₆ integrin but does not inhibit substantially avβ₈ integrin. In a further such method, the compound inhibits αvβ₆ integrin but does not inhibit substantially α₂β₃ integrin. In one embodiment is provided a method of inhibiting αvβ₆ integrin and one or more of avβ₁, αvβ₃, αvβ₅, α₂β₁, α₃β₁, α₆β₁, α₇β₁ and α₁₁β₁ integrin in an individual in need thereof. In another embodiment is provided a method of inhibiting αvβ₆ integrin and αvβ₁ integrin. In another embodiment is provided a method of inhibiting αvβ₆ integrin, avβ₃ integrin and avβ₅ integrin. In another embodiment is provided a method of inhibiting αvβ₆ integrin and a₂β₁ integrin. In another embodiment is provided a method of inhibiting αvβ₆ integrin, a₂β₁ integrin and a₃(3i integrin. In another embodiment is provided a method of inhibiting αvβ₆ integrin and α₆β₁ integrin. In another embodiment is provided a method of inhibiting αvβ₆ integrin and a₇β₁ integrin. In another embodiment is provided a method of inhibiting αvβ₆ integrin and a₁₁β₁ integrin. In all such embodiments, in one aspect the method of inhibition is for an individual in need thereof, such as an individual who has or is suspected of having a fibrotic disease, and wherein the method comprises administering to the individual a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-66 in FIG. 1, or a stereoisomer thereof, or a pharmaceutically acceptable salt thereof. In all such embodiments, in one aspect the method of inhibition is for an individual in need thereof, such as an individual who has or is suspected of having a fibrotic disease, and wherein the method comprises administering to the individual a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-147, or a stereoisomer thereof, or a pharmaceutically acceptable salt thereof. In all such embodiments, in one aspect the method of inhibition is for an individual in need thereof, such as an individual who has or is suspected of having a fibrotic disease, and wherein the method comprises administering to the individual a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-665, or a stereoisomer thereof, or a pharmaceutically acceptable salt thereof. In all such embodiments, in one aspect the method of inhibition is for an individual in need thereof, such as an individual who has or is suspected of having a fibrotic disease, and wherein the method comprises administering to the individual a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof.

Compounds of formula (A) can be used in any of the compositions, methods, and uses recited herein for formula (I) and variations of formula (I).

In any of the described methods, in one aspect the individual is a human, such as a human in need of the method. The individual may be a human who has been diagnosed with or is suspected of having a fibrotic disease. The individual may be a human who does not have detectable disease but who has one or more risk factors for developing a fibrotic disease.

Also provided herein are dosage forms configured for daily administration, comprising a pharmaceutically acceptable carrier or excipient; and a unit dose of a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof.

A unit dose, such as a unit dose for daily administration, can comprise about 1, 2.5, 5, 7.5, 10, 15, 20, 25, 30, 35, 40, 50, 75, 80, 85, 90, 95, 100, 105, 110, 115, 120, or 125 mg of the compound, or a range between any two of the preceding values, such as about 1-125, 1-5, 2.5-7.5, 5-15, 10-15, 10-20, 10-25, 10-30, 10-35, 10-40, 10-50, 10-75, 15-20, 15-25, 15-30, 15-35, 15-40, 15-50, 15-75, 20-25, 20-30, 20-35, 20-40, 20-50, 20-75, 25-30, 25-35, 25-40, 25-50, 25-75, 30-35, 30-40, 30-50, 30-75, 35-40, 35-50, 35-75, 40-50, 40-75, 50-75, 50-100, 60-85, 70-90, 70-100, 80-125, 90-125, or 100-125 mg.

A unit dose, such as a unit dose for daily administration, can comprise about 1, 2.5, 5, 7.5, 10, 15, 20, 25, 30, 35, 40, 50, 75, 80, 85, 90, 95, 100, 105, 110, 115, 120, 125, 150, 175, 200, 225, or 250 mg of the compound, or a range between any two of the preceding values, such as about 1-125, 1-250, 1-5, 2.5-7.5, 5-15, 10-15, 10-20, 10-25, 10-30, 10-35, 10-40, 10-50, 10-75, 15-20, 15-25, 15-30, 15-35, 15-40, 15-50, 15-75, 20-25, 20-30, 20-35, 20-40, 20-50, 20-75, 25-30, 25-35, 25-40, 25-50, 25-75, 30-35, 30-40, 30-50, 30-75, 35-40, 35-50, 35-75, 40-50, 40-75, 50-75, 50-100, 50-150, 50-250, 60-85, 70-90, 70-100, 80-125, 90-125, 100-125, 100-150, 100-200, 125-175, 100-225, 100-250, and 150-250 mg. For example, the unit dose may be 10 mg. The unit dose may be 15 mg. The unit dose may be 20 mg. The unit dose may be 30 mg. The unit dose may be 40 mg. The unit dose may be 50 mg. The unit dose may be 60 mg. The unit dose may be 70 mg. The unit dose may be 75 mg. The unit dose may be 80 mg. The unit dose may be 90 mg. The unit dose may be 100 mg. The unit dose may be 110 mg. The unit dose may be 120 mg. The unit dose may be 125 mg. The unit dose may be 150 mg. The unit dose may be 175 mg. The unit dose may be 200 mg. The unit dose may be 225 mg. The unit dose may be 250 mg.

A unit dose, such as a unit dose for daily administration, can comprise the compound in an amount effective on administration to an individual to produce a Cₘₐₓ in plasma of the individual in ng/mL of at least about, or greater than about, one of: 700, 750, 800, 850, 900, 950, 1000, 1050, 1100, 1150, 1200, 1250, 1300, 1350, 1400, 1450, or 1500; or a range between any two of the preceding concentrations, such as 700-1500, 700-900, 800-1300, 750-950, 800-1000, 850-950, 850-1050, 900-1400, 900-1300, 900-1200, 900-1100, 950-1050, 950-1400, 950-1150, 1000-1400, 1000-1300, 1000-1200, and the like. For example, Cₘₐₓ can be about 700 ng/mL or greater. Cₘₐₓ can be about 750 ng/mL or greater. Cₘₐₓ can be about 800 ng/mL or greater. Cₘₐₓ can be about about 850 ng/mL or greater. Cₘₐₓ can be 900 ng/mL or greater. Cₘₐₓ can be about 950 ng/mL or greater. Cₘₐₓ can be about 1000 ng/mL or greater. Cₘₐₓ can be about 1050 ng/mL or greater. Cₘₐₓ can be about 1100 ng/mL or greater. Cₘₐₓ can be about 1200 ng/mL or greater. Cₘₐₓ can be about 1300 ng/mL or greater. Cₘₐₓ can be about 1400 ng/mL or greater.Cₘₐₓ can be about 1500 ng/mL or greater.

A unit dose, such as a unit dose for daily administration, can comprise the compound in an amount effective on administration to an individual to produce a Cₘₐₓ in ng/mL in plasma of the individual, the Cₘₐₓ corresponding to a plasma-adjusted concentration effective to inhibit a percentage of αvβ₆ or αvβ₁ in the individual of at least about one of 50, 55, 60, 65, 70, 75, 80, 85, 90, 95, or 100, or a range between any two of the preceding percentages, for example, 50-100, 60-90, 70-90, 75-95, and the like. In some embodiments, the compound may be a dual αvβ₆ and αvβ₁ inhibitor, and the Cₘₐₓ can correspond to a plasma-adjusted concentration effective to inhibit a percentage of each of αvβ₆ and αvβ₁ in the individual, each percentage independently selected from the preceding percentages, or a range between any two of the preceding percentages. For example, the plasma-adjusted concentration can be effective to inhibit αvβ₆ by at least about 50%. The plasma-adjusted concentration can be effective to inhibit αvβ₆ by at least about 60%. The plasma-adjusted concentration can be effective to inhibit αvβ₆ by at least about 70%. The plasma-adjusted concentration can be effective to inhibit αvβ₆ by at least about 80%. The plasma-adjusted concentration can be effective to inhibit αvβ₆ by at least about 90%. Further, for example, the plasma-adjusted concentration can be effective to inhibit avβ₁ by at least about 50%. The plasma-adjusted concentration can be effective to inhibit avβ₁ by at least about 60%. The plasma-adjusted concentration can be effective to inhibit avβ₁ by at least about 70%. The plasma-adjusted concentration can be effective to inhibit avβ₁ by at least about 80%. The plasma-adjusted concentration can be effective to inhibit avβ₁ by at least about 90%. The recitation “percentage of each of αvβ₆ and/or avβ₁ in the subject, each percentage independently selected” means, in the alternative, a single αvβ₆ inhibitor and corresponding percentage, a single αvβ₁ inhibitor and corresponding percentage, or a dual avβ₆/avβ₆ inhibitor and corresponding independently selected percentages.

Also provided herein are dosage forms configured for daily administration, comprising a pharmaceutically acceptable carrier or excipient; and a unit dose of a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof.

In various embodiments, a dose, e.g., a unit dose, such as a unit dose for daily administration, can include the compound in an amount of one of, or one of about: 1, 2.5, 5, 7.5, 10, 15, 20, 25, 30, 35, 40, 45, 50, 55, 60, 75, 80, 85, 90, 95, 100, 105, 110, 115, 120, 125, 130, 140, 150, 160, 170, 175, 180, 190, 200, 225, 240, 250, 275, 300, 320, 325, 350, 375, 400, 425, 450, 475, 480, 500, 525, 550, 560, 575, 600, 625, 640, 650, 675, 700, 720, 725, 750, 775, 800, 825, 850, 875, 880, 900, 925, 950, 960, 975, 1000, 1025, or 1040 milligrams. For example, a dose can include the compound in an amount of, or of about, 10 mg. A dose can include the compound in an amount of, or of about, 15 mg. A dose can include the compound in an amount of, or of about, 20 mg. A dose can include the compound in an amount of, or of about, 30 mg. A dose can include the compound in an amount of, or of about, 40 mg. A dose can include the compound in an amount of, or of about, 50 mg. A dose can include the compound in an amount of, or of about, 75 mg. A dose can include the compound in an amount of, or of about, 80 mg. A dose can include the compound in an amount of, or of about, 100 mg. A dose can include the compound in an amount of, or of about, 120 mg. A dose can include the compound in an amount of, or of about, 160 mg. A dose can include the compound in an amount of, or of about, 240 mg. A dose can include the compound in an amount of, or of about, 320 mg. A dose can include the compound in an amount of, or of about, 400 mg. A dose can include the compound in an amount of, or of about, 480 mg. A dose can include the compound in an amount of, or of about, 560 mg. A dose can include the compound in an amount of, or of about, 640 mg. A dose can include the compound in an amount of, or of about, 720 mg. A dose can include the compound in an amount of, or of about, 800 mg. A dose can include the compound in an amount of, or of about, 880 mg. A dose can include the compound in an amount of, or of about, 960 mg. A dose can include the compound in an amount of, or of about, 1040 mg.

In various embodiments, a dose, e.g., a unit dose, such as a unit dose for daily administration, can include the compound in an amount comprising an amount of the compound in mg of about one of about: 320, 400, 480, 560, 640, 720, 800, 880, 960, or 1040, or a range between any two of the preceding values.

In various embodiments, a dose, e.g., a unit dose, such as a unit dose for daily administration, can include the compound in an amount comprising an amount of the compound in mg of about one of about: 400, 480, 560, 640, 720, 800, 880, 960, or 1040.

In various embodiments, a dose, e.g., a unit dose, such as a unit dose for daily administration, can include the compound in an amount comprising an amount of the compound in mg of a range between about 320 and any one of about 400, 480, 560, 640, 720, 800, 880, 960, or 1040.

In various embodiments, a dose, e.g., a unit dose, such as a unit dose for daily administration, can include the compound in an amount comprising an amount of the compound in mg of about one of: 400, 480, 560, 640, 720, 800, 880, 960, or 1040, or a range between any two of the preceding values.

In some embodiments, the weight dosage of a pharmaceutically acceptable salt is adjusted to administer the same amount of active agent on a molar basis as would be administered if the non-salt compound were used. For example, if a dosage is indicated as 100 mg of a non-salt compound with a molecular weight of 500, which is a dosage of 0.2 mmol, and the hydrochloride salt of the same compound has a molecular weight of 536.5, then 107.3 mg of the hydrochloride salt would be administered in order to administer 0.2 mmol of active agent.

In some embodiments, the unit dose may include the compound in a percentage range about any of the individual values in milligrams recited in the preceding paragraph, for example, any percentage range independently selected from one of, or one of about: ± 1%, ± 2%, ± 2.5%, ± 5%, ± 7.5%, ± 10%, ± 15%, ± 20%, ± 25%, ± 30%, ± 40%, or ± 50%. For example, the range may be, or be about, ± 1%. The range may be, or be about, ± 2%. The range may be, or be about, ± 2.5%. The range may be, or be about, ± 5%. The range may be, or be about, ± 7.5%. The range may be, or be about, ± 10%. The range may be, or be about, ± 15%. The range may be, or be about, ± 20%. The range may be, or be about, ± 25%. The range may be, or be about, ± 30%. The range may be, or be about, ± 40%. The range may be, or be about, ± 50%.

Further, for example, the unit dose may include the compound in an amount of one of: 10 mg ± 1%; 10 mg ± 2%; 10 mg ± 2.5%; 10 mg ± 5%; 10 mg ± 7.5%; 10 mg ± 10%; 10 mg ± 15%; 10 mg ± 20%; 10 mg ± 25%; 10 mg ± 30%; 10 mg ± 40%; or 10 mg ± 50%. The unit dose may include the compound in an amount of one of: 15 mg ± 1%; 15 mg ± 2%; 15 mg ± 2.5%; 15 mg ± 5%; 15 mg ± 7.5%; 15 mg ± 10%; 15 mg ± 15%; 15 mg ± 20%; 15 mg ± 25%; 15 mg ± 30%; 15 mg ± 40%; or 15 mg ± 50%. The unit dose may include the compound in an amount of one of: 20 mg ± 1%; 20 mg ± 2%; 20 mg ± 2.5%; 20 mg ± 5%; 20 mg ± 7.5%; 20 mg ± 10%; 20 mg ± 15%; 20 mg ± 20%; 20 mg ± 25%; 20 mg ± 30%; 20 mg ± 40%; or 20 mg ± 50%. The unit dose may include the compound in an amount of one of: 30 mg ± 1%; 30 mg ± 2%; 30 mg ± 2.5%; 30 mg ± 5%; 30 mg ± 7.5%; 30 mg ± 10%; 30 mg ± 15%; 30 mg ± 20%; 30 mg ± 25%; 30 mg ± 30%; 30 mg ± 40%; or 30 mg ± 50%. The unit dose may include the compound in an amount of one of: 40 mg ± 1%; 40 mg ± 2%; 40 mg ± 2.5%; 40 mg ± 5%; 40 mg ± 7.5%; 40 mg ± 10%; 40 mg ± 15%; 40 mg ± 20%; 40 mg ± 25%; 40 mg ± 30%; 40 mg ± 40%; or 40 mg ± 50%. The unit dose may include the compound in an amount of one of: 50 mg ± 1%; 50 mg ± 2%; 50 mg ± 2.5%; 50 mg ± 5%; 50 mg ± 7.5%; 50 mg ± 10%; 50 mg ± 15%; 50 mg ± 20%; 50 mg ± 25%; 50 mg ± 30%; 50 mg ± 40%; or 50 mg ± 50%. The unit dose may include the compound in an amount of one of: 60 mg ± 1%; 60 mg ± 2%; 60 mg ± 2.5%; 60 mg ± 5%; 60 mg ± 7.5%; 60 mg ± 10%; 60 mg ± 15%; 60 mg ± 20%; 60 mg ± 25%; 60 mg ± 30%; 60 mg ± 40%; or 60 mg ± 50%. The unit dose may include the compound in an amount of one of: 75 mg ± 1%; 75 mg ± 2%; 75 mg ± 2.5%; 75 mg ± 5%; 75 mg ± 7.5%; 75 mg ± 10%; 75 mg ± 15%; 75 mg ± 20%; 75 mg ± 25%; 75 mg ± 30%; 75 mg ± 40%; or 75 mg ± 50%. The unit dose may include the compound in an amount of one of: 80 mg ± 1%; 80 mg ± 2%; 80 mg ± 2.5%; 80 mg ± 5%; 80 mg ± 7.5%; 80 mg ± 10%; 80 mg ± 15%; 80 mg ± 20%; 80 mg ± 25%; 80 mg ± 30%; 80 mg ± 40%; or 80 mg ± 50%. The unit dose may include the compound in an amount of one of: 100 mg ± 1%; 100 mg ± 2%; 100 mg ± 2.5%; 100 mg ± 5%; 100 mg ± 7.5%; 100 mg ± 10%; 100 mg ± 15%; 100 mg ± 20%; 100 mg ± 25%; 100 mg ± 30%; 100 mg ± 40%; or 100 mg ± 50%. The unit dose may include the compound in an amount of one of: 120 mg ± 1%; 120 mg ± 2%; 120 mg ± 2.5%; 120 mg ± 5%; 120 mg ± 7.5%; 120 mg ± 10%; 120 mg ± 15%; 120 mg ± 20%; 120 mg ± 25%; 120 mg ± 30%; 120 mg ± 40%; or 120 mg ± 50%. The unit dose may include the compound in an amount of one of: 160 mg ± 1%; 160 mg ± 2%; 160 mg ± 2.5%; 160 mg ± 5%; 160 mg ± 7.5%; 160 mg ± 10%; 160 mg ± 15%; 160 mg ± 20%; 160 mg ± 25%; 160 mg ± 30%; 160 mg ± 40%; or 160 mg ± 50%. The unit dose may include the compound in an amount of one of: 240 mg ± 1%; 240 mg ± 2%; 240 mg ± 2.5%; 240 mg ± 5%; 240 mg ± 7.5%; 240 mg ± 10%; 240 mg ± 15%; 240 mg ± 20%; 240 mg ± 25%; 240 mg ± 30%; 240 mg ± 40%; or 240 mg ± 50%. The unit dose may include the compound in an amount of one of: 320 mg ± 1%; 320 mg ± 2%; 320 mg ± 2.5%; 320 mg ± 5%; 320 mg ± 7.5%; 320 mg ± 10%; 320 mg ± 15%; 320 mg ± 20%; 320 mg ± 25%; 320 mg ± 30%; 320 mg ± 40%; or 320 mg ± 50%. The unit dose may include the compound in an amount of one of: 400 mg ± 1%; 400 mg ± 2%; 400 mg ± 2.5%; 400 mg ± 5%; 400 mg ± 7.5%; 400 mg ± 10%; 400 mg ± 15%; 400 mg ± 20%; 400 mg ± 25%; 400 mg ± 30%; 400 mg ± 40%; or 400 mg ± 50%. The unit dose may include the compound in an amount of one of: 480 mg ± 1%; 480 mg ± 2%; 480 mg ± 2.5%; 480 mg ± 5%; 480 mg ± 7.5%; 480 mg ± 10%; 480 mg ± 15%; 480 mg ± 20%; 480 mg ± 25%; 480 mg ± 30%; 480 mg ± 40%; or 480 mg ± 50%. The unit dose may include the compound in an amount of one of: 560 mg ± 1%; 560 mg ± 2%; 560 mg ± 2.5%; 560 mg ± 5%; 560 mg ± 7.5%; 560 mg ± 10%; 560 mg ± 15%; 560 mg ± 20%; 560 mg ± 25%; 560 mg ± 30%; 560 mg ± 40%; or 560 mg ± 50%. The unit dose may include the compound in an amount of one of: 640 mg ± 1%; 640 mg ± 2%; 640 mg ± 2.5%; 640 mg ± 5%; 640 mg ± 7.5%; 640 mg ± 10%; 640 mg ± 15%; 640 mg ± 20%; 640 mg ± 25%; 640 mg ± 30%; 640 mg ± 40%; or 640 mg ± 50%. The unit dose may include the compound in an amount of one of: 720 mg ± 1%; 720 mg ± 2%; 720 mg ± 2.5%; 720 mg ± 5%; 720 mg ± 7.5%; 720 mg ± 10%; 720 mg ± 15%; 720 mg ± 20%; 720 mg ± 25%; 720 mg ± 30%; 720 mg ± 40%; or 720 mg ± 50%. The unit dose may include the compound in an amount of one of: 800 mg ± 1%; 800 mg ± 2%; 800 mg ± 2.5%; 800 mg ± 5%; 800 mg ± 7.5%; 800 mg ± 10%; 800 mg ± 15%; 800 mg ± 20%; 800 mg ± 25%; 800 mg ± 30%; 800 mg ± 40%; or 800 mg ± 50%. The unit dose may include the compound in an amount of one of: 880 mg ± 1%; 880 mg ± 2%; 880 mg ± 2.5%; 880 mg ± 5%; 880 mg ± 7.5%; 880 mg ± 10%; 880 mg ± 15%; 880 mg ± 20%; 880 mg ± 25%; 880 mg ± 30%; 880 mg ± 40%; or 880 mg ± 50%. The unit dose may include the compound in an amount of one of: 960 mg ± 1%; 960 mg ± 2%; 960 mg ± 2.5%; 960 mg ± 5%; 960 mg ± 7.5%; 960 mg ± 10%; 960 mg ± 15%; 960 mg ± 20%; 960 mg ± 25%; 960 mg ± 30%; 960 mg ± 40%; or 960 mg ± 50%. The unit dose may include the compound in an amount of one of: 1040 mg ± 1%; 1040 mg ± 2%; 1040 mg ± 2.5%; 1040 mg ± 5%; 1040 mg ± 7.5%; 1040 mg ± 10%; 1040 mg ± 15%; 1040 mg ± 20%; 1040 mg ± 25%; 1040 mg ± 30%; 1040 mg ± 40%; or 1040 mg ± 50%.

A unit dose, such as a unit dose for daily administration, can comprise the compound in an amount effective on administration to an individual to produce a Cₘₐₓ in plasma of the individual in ng/mL of at least about, or greater than about, one of: 700, 750, 800, 850, 900, 950, 1000, 1050, 1100, 1150, 1200, 1250, 1300, 1350, 1400, 1450, 1500, 1600, 1700, 1800, 1900, 2000, 2100, 2200, 2300, 2400, or 2500; or a range between any two of the preceding concentrations, such as 700-1500, 700-900, 800-1300, 750-950, 800-1000, 850-950, 850-1050, 900-1400, 900-1300, 900-1200, 900-1100, 950-1050, 950-1400, 950-1150, 1000-1400, 1000-1300, 1000-1200, 700-2500, 1000-2500, 1500-2500, 1500-2000, 1500-2500, 2000-2500, and the like. For example, Cₘₐₓ can be, or be about, about 700 ng/mL or greater. Cₘₐₓ can be, or be about, about 750 ng/mL or greater. Cₘₐₓ can be, or be about, about 800 ng/mL or greater. Cₘₐₓ can be, or be about, 850 ng/mL or greater. Cₘₐₓ can be, or be about, 900 ng/mL or greater. Cₘₐₓ can be, or be about, 950 ng/mL or greater. Cₘₐₓ can be, or be about, 1000 ng/mL or greater. Cₘₐₓ can be, or be about, 1050 ng/mL or greater. Cₘₐₓ can be, or be about, 1100 ng/mL or greater. Cₘₐₓ can be, or be about, 1200 ng/mL or greater. Cₘₐₓ can be, or be about, 1300 ng/mL or greater. Cₘₐₓ can be, or be about, 1400 ng/mL or greater. Cₘₐₓ can be, or be about, 1500 ng/mL or greater. Cₘₐₓ can be, or be about, 1600 ng/mL or greater. Cₘₐₓ can be, or be about, 1700 ng/mL or greater. Cₘₐₓ can be, or be about, 1800 ng/mL or greater. Cₘₐₓ can be, or be about, 1900 ng/mL or greater. Cₘₐₓ can be, or be about, 2000 ng/mL or greater. Cₘₐₓ can be, or be about, 2100 ng/mL or greater. Cₘₐₓ can be, or be about, 2200 ng/mL or greater. Cₘₐₓ can be, or be about, 2300 ng/mL or greater. Cₘₐₓ can be, or be about, 2400 ng/mL or greater. Cₘₐₓ can be, or be about, 2500 ng/mL or greater.

A unit dose, such as a unit dose for daily administration, can comprise the compound in an amount effective on administration to an individual to produce a Cₘₐₓ in plasma of the individual in ng/mL of at least about one of 700, 750, 800, 850, 900, 950, 1000, 1050, 1100, 1150, 1200, 1250, 1300, 1350, 1400, 1450, or 1500, or a range between any two of the preceding concentrations

A unit dose, such as a unit dose for daily administration, can comprise the compound in an amount effective on administration to an individual to produce a Cₘₐₓ in plasma of the individual in ng/mL in a range between of at least about any one of 700, 750, 800, 850, 900, 950, 1000, 1050, 1100, 1150, 1200, 1250, 1300, 1350, 1400, or 1450 as a lower limit and 1500 as an upper limit.

A unit dose, such as a unit dose for daily administration, can comprise the compound in an amount effective on administration to an individual to produce a Cₘₐₓ in plasma of the individual in ng/mL of at least about one of: 1500, 1600, 1700, 1800, 1900, 2000, 2100, 2200, 2300, 2400, or 2500, or a range between any two of the preceding concentrations;

A unit dose, such as a unit dose for daily administration, can comprise the compound in an amount effective on administration to an individual to produce a Cₘₐₓ in plasma of the individual in ng/mL of at least about one of: 1600, 1700, 1800, 1900, 2000, 2100, 2200, 2300, 2400, or 2500, or a range between any two of the preceding concentrations;

A unit dose, such as a unit dose for daily administration, can comprise the compound in an amount effective on administration to an individual to produce a Cₘₐₓ in plasma of the individual in ng/mL in a range between at least 1500 and any one of 1600, 1700, 1800, 1900, 2000, 2100, 2200, 2300, 2400, or 2500.

A unit dose, such as a unit dose for daily administration, can comprise the compound in an amount effective on administration to an individual to produce a Cₘₐₓ in ng/mL in plasma of the individual, the Cₘₐₓ corresponding to a plasma-adjusted concentration effective to inhibit a percentage of αvβ₆ or αvβ₁ in the individual of at least one of, or at least about one of: 50, 55, 60, 65, 70, 75, 80, 85, 90, 95, 97, 98, 99, or 100, or a range between any two of the preceding percentages, for example, 50-100, 60-90, 70-90, 75-95, 90-95, 90-98, 90-99, and the like. In some embodiments, the compound may be a dual αvβ₆ and αvβ₁ inhibitor, and the Cₘₐₓ can correspond to a plasma-adjusted concentration effective to inhibit a percentage of each of αvβ₆ and αvβ₁ in the individual, each percentage independently selected from the preceding percentages, or a range between any two of the preceding percentages. For example, the plasma-adjusted concentration can be effective to inhibit αvβ₆ by at least about 50%. The plasma-adjusted concentration can be effective to inhibit αvβ₆ by at least about 60%. The plasma-adjusted concentration can be effective to inhibit αvβ₆ by at least about 70%. The plasma-adjusted concentration can be effective to inhibit αvβ₆ by at least about 80%. The plasma-adjusted concentration can be effective to inhibit αvβ₆ by at least about 90%. The plasma-adjusted concentration can be effective to inhibit αvβ₆ by at least about 95%. The plasma-adjusted concentration can be effective to inhibit αvβ₆ by at least about 97%. The plasma-adjusted concentration can be effective to inhibit αvβ₆ by at least about 98%. The plasma-adjusted concentration can be effective to inhibit αvβ₆ by at least about 99%. The plasma-adjusted concentration can be effective to inhibit αvβ₆ by about 100%. Further, for example, the plasma-adjusted concentration can be effective to inhibit avβ₁ by at least about 50%. The plasma-adjusted concentration can be effective to inhibit αvβ₁ by at least about 60%. The plasma-adjusted concentration can be effective to inhibit αvβ₁ by at least about 70%. The plasma-adjusted concentration can be effective to inhibit αvβ₁ by at least about 80%. The plasma-adjusted concentration can be effective to inhibit αvβ₁ by at least about 90%. The plasma-adjusted concentration can be effective to inhibit αvβ₁ by at least about 95%. The plasma-adjusted concentration can be effective to inhibit αvβ₁ by at least about 97%. The plasma-adjusted concentration can be effective to inhibit αvβ₁ by at least about 98%. The plasma-adjusted concentration can be effective to inhibit αvβ₁ by at least about 99%. The plasma-adjusted concentration can be effective to inhibit αvβ₁ by about 100%. The recitation “percentage of each of αvβ₆ and/or avβ₁ in the subject, each percentage independently selected” means, in the alternative, a single αvβ₆ inhibitor and corresponding percentage, a single αvβ₁ inhibitor and corresponding percentage, or a dual αvβ₆/avβ₆ inhibitor and corresponding independently selected percentages.

The dosage form for daily administration can be administered to an individual in need thereof once daily. That is, the total amount of a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof, which is to be administered each day, can be administered all together at one time daily. Alternatively, if it is desirable that the total amount of a compound of formula (A), formula (I), or any variation thereof, e.g., a compound of formula (I-A), (I-B), (I-C), (I-D), (I-E), (I-F), (I-G), (I-H), (II), (II-A), (II-B), (II-C), (II-D), (II-E), (II-F), (II-G), or (II-H), a compound selected from Compound Nos. 1-780, or a stereoisomer thereof, or a pharmaceutically acceptable salt thereof, is to be administered in two or more portions daily, the dosage form containing the appropriate amount of compound can be administered two times or more daily, such as twice a day, three times a day, or four times a day.

The present application contemplates combination administration of the compound of Formula (A), (I), or (II), or a salt thereof with a second drug, e.g., as described in any of Enumerated Embodiments 1-83. Such combined administration includes as the second drug any aspect of pirfenidone, salts thereof, pharmaceutical formulations or dosage forms thereof, and related methods as described in U.S. Pat. Nos. 7,566,729, 7,635,707, 7,696,236, 7,767,225, 7,767,700, 7,816,383, 7,910,610, 7,988,994, 8,013,002, 8,084,475, 8,318,780, 8,383,150, 8420674, 8592462, 8609701, 8,648,098, 8,753,679, 8,754,109, 8,778,947, 9,561,217, 10,188,637, and in each of the FDA approvals/labels/inserts for New Drug Application 208780 for ESBRIET® (pirfenidone) oral capsules and oral film-coated tablets, the approvals/labels/inserts dating from Jan. 11, 2017 to Jul. 31, 2019, accessed Oct. 1, 2021 at URL www.accessdata.fda.gov/scripts/cder/dai7index.cfm? event=overview.process&ApplNo=2087 80. The entire contents of each of the preceding documents are incorporated herein by reference.

A deuterated analog of pirfenidone has been reported to have a more favorable adverse event profile than pirfenidone (Chen et al., Clin Pharmacol Drug Dev. 2022 Feb; 11(2):220-234. doi: 10. 1002/cpdd. 1040). Deuterated pirfenidone analogs as disclosed in that publication, and in U.S. Pat. Application Publication Nos. 2020/0093810 and 2021/0205283 and International Patent Application No. WO 2020/056430, can be used as the pirfenidone component in any of the compositions disclosed herein. In particular, the following deuterated analog can be used:

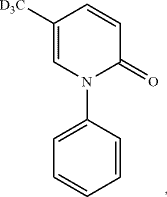

or a pharmaceutically acceptable salt thereof.

The present application also contemplates combination administration of the compound of Formula (A), (I), or (II), or a salt thereof with a second drug, e.g., as described in any of Enumerated Embodiments 1-83. Such combined administration includes as the second drug any aspect of nintedanib, salts thereof, pharmaceutical formulations or dosage forms thereof, and related methods as described in U.S. Pat. Nos. 6,762,180, 7,119,093, 9,907,756, 10,105,323, or 10,154,990, and in each of the FDA-approved labels or inserts for New Drug Application 205832 for OFEV® (nintedanib) oral capsules, the labels dating from Oct. 15, 2014 to Oct. 28, 2020, accessed Oct. 1, 2021 at URL www.accessdata.fda.gov/scripts/cder/daf/index.cfm?event=overview.process&ApplNo=2058 32. The entire contents of each of the preceding documents are incorporated herein by reference.

In another aspect, method of treating a subject for a disease is provided, the method comprising: administering to the subject a first drug that comprising a compound of formula (A) or a salt thereof; and administering to the subject at least a second drug that is selected from the group consisting of: pirfenidone and nintedanib, or a salt thereof, whereby the subject is treated for the disease. In some embodiments, the compound of formula (A) is represented by Formula (I). In some embodiments, the compound of formula (A) is represented by Formula (II). In some embodiments, the second drug is pirfenidone, a salt thereof, a pharmaceutical formulation or dosage form thereof. In some embodiments, the second drug is nintedanib, a salt thereof, a pharmaceutical formulation or dosage form thereof.

Administration of any drug, such as pirfenidone or of nintedanib, can be associated with adverse events (AEs), which may rise to the level of serious adverse events (SAEs). A common AE associated with pirfenidone and nintedanib is gastrointestinal distress, such as diarrhea, which in many patients rises to the level of an SAE. Notably, while combinations of Compound 5 and nintedanib or of Compound 5 and pirfenidone resulted in treatment-emergent adverse events (TEAEs) in patients, administration of Compound 5 alone did not result in serious adverse events.

### Salts and Polymorphs of Compound 5

Various salts and polymorphs of Compound 5, (S)-4-((2-methoxyethyl) (4-(5,6,7,8-tetrahydro-1,8-naphthyridin-2-yl) butyl)amino)-2-(quinazolin-4-ylamino) butanoic acid, can be used in the compositions and methods disclosed herein, such as combinations of Compound 5 with pirfenidone or combinations of Compound 5 with nintedanib.

Salts and polymorphs of Compound 5 are disclosed in U.S. Pat. Application Publication No. 2022/0177468. Phosphate salts of Compound 5 are preferred. For example, in one embodiment, the crystalline Form I phosphate salt of Compound 5 as described in Example 4 can be used. In one embodiment, the crystalline Form IV phosphate salt of Compound 5 as described in Example 7 of US 2022/0177468. The crystalline Form II fumarate salt of Example 5 or the crystalline Form III naphthalenedisulfonic acid salt of Example 6 of US 2022/0177468 can also be used. Compound 5 can also be used in zwitterionic form. Compound 5 can also be used in amorphous form.

In some embodiments, Compound 5 is the only therapy specific for a lung disorder (that is, the only lung-specific therapy) administered to the subject, such as a subject with a fibrotic lung disease, for example, idiopathic pulmonary fibrosis. For example, Compound 5 may be administered without administration of pirfenidone or nintedanib. For example, Compound 5 may be administered without administration of pirfenidone, nintedanib, or any other therapy specific for a lung disorder. For example, Compound 5 may be administered without administration of pirfenidone, nintedanib, or any other therapy specific for a fibrotic lung disorder. For example, Compound 5 may be administered without administration of pirfenidone, nintedanib, or any other therapy specific for idiopathic pulmonary fibrosis. Subjects may be taking medications for reasons other than treatment of lung disorders, or which are not specific for lung disorders, such as over-the counter treatments, including, but not limited to, vitamins, minerals, or ibuprofen, or prescription treatments such as drugs to treat diabetes, high blood pressure, or other disorders.

In some embodiments, Compound 5 is administered to a subject without serious adverse events. In some embodiments, Compound 5 is administered to a subject without treatment-emergent adverse event. In some embodiments, Compound 5 is administered to a subject with less than about a 20% probability, less than about a 10% probability, or less than about a 5% probability of serious adverse events. In some embodiments, Compound 5 is administered to a subject with less than about a 20% probability, less than about a 10% probability, or less than about a 5% probability of treatment-emergent adverse events. Probability of a serious adverse event or of a treatment-emergent adverse event can be calculated from the percentage of such events in a group of patients treated with Compound 5. In any of these embodiments, Compound 5 can be the only therapy specific for a lung disorder (that is, the only lung-specific therapy) administered to the subject, such as a subject with a fibrotic lung disease, for example, idiopathic pulmonary fibrosis.

### Amelioration of Decline of Forced Vital Capacity

Forced vital capacity (FVC) is the maximum volume of air that a person can exhale from their lungs after taking the deepest breath possible. Lung diseases such as idiopathic pulmonary fibrosis (IPF), chronic obstructive pulmonary disease, asbestosis, and many other disorders can affect FVC. The decrease in FVC in a subject over time is used to measure the progression of diseases such as idiopathic pulmonary fibrosis.

Reducing or slowing the decline in forced vital capacity is an important measure of treatment efficacy in idiopathic pulmonary fibrosis. Increasing FVC would provide even more benefit to the subject. “Amelioration of decline of forced vital capacity” is a generic term for either reducing the decline in forced vital capacity or increasing forced vital capacity.

Disclosed herein are methods of amelioration of decline of forced vital capacity, comprising administering one or more of Compounds 1-780 to a subject in need thereof, such as Compound 5, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, including a phosphate salt thereof, such as a phosphate salt in crystalline form. The amerlioration can be a reduction in the decline in forced vital capacity. The amelioration can be an increase in forced vital capacity, such as at least a partial restoration of FVC lost prior to commencement of the administration of one or more of Compounds 1-780, such as Compound 5. Administration of one or more of Compounds 1-780, such as Compound 5, can occur for at least about 12 weeks, at least about 24 weeks, at least about 36 weeks, at least about 1 year, at least about 2 years, at least about 3 years, at least about 4 years, at least about 5 years, at least about 10 years, or indefinitely over a subject’s lifetime, such as about 12 weeks to about 24 weeks, about 12 weeks to about 36 weeks, about 12 weeks to about 1 year, about 12 weeks to about 2 years, about 12 weeks to about 3 years, about 12 weeks to about 4 years, about 12 weeks to about 5 years, about 12 weeks to about 10 years, or for at least about 12 weeks to be continued indefinitely, such as life-long treatment.

Amelioration of the decline in forced vital capacity can be a reduction of decline in forced vital capacity of about 50 mL or less, about 30 mL or less, or about 15 mL or less. The reduction of decline in forced vital capacity can be about 50 mL or less, about 30 mL or less, or about 15 mL or less over a period of about 12 weeks. The reduction of decline in forced vital capacity can be about 50 mL to about 1 mL, about 30 mL to 1 mL, or about 15 mL to about 1 mL. The reduction in decline in forced vital capacity can be about 50 mL to about 1 mL, about 30 mL to 1 mL, or about 15 mL to about 1 mL over a period of about 12 weeks. The reduction in decline in forced vital capacity can be as compared to the decline in a subject who is not treated by administration of one or more of Compounds 1-780, such as Compound 5, or compared to an average decline in a group of subjects who are not treated by administration of one or more of Compounds 1-780, such as Compound 5. By way of illustration, if a subject who is administered one or more of Compounds 1-780 has a decline of forced vital capacity over a certain period, such as about 12 weeks, of about 25 mL, and a subject who is not administered one or more of Compounds 1-780 has a decline of forced vital capacity over the same period of about 75 mL, then the reduction in decline of forced vital capacity is 50 mL over the period.

Amelioration of the decline in forced vital capacity can be an increase in forced vital capacity of about 10 mL or more, about 20 mL or more, about 30 mL or more, about 40 mL or more, about 50 mL or more, about 60 mL or more, about 70 mL or more, about 80 mL or more, about 90 mL or more, about 100 mL or more, about 110 mL or more, about 120 mL or more, about 130 mL or more, about 140 mL or more, about 150 mL or more, about 160 mL or more, about 170 mL or more, about 180 mL or more, or about 185 mL or more, or between a range of any two of the foregoing values. The increase in forced vital capacity can be about 10 mL or more, about 20 mL or more, about 30 mL or more, about 40 mL or more, about 50 mL or more, about 60 mL or more, about 70 mL or more, about 80 mL or more, about 90 mL or more, about 100 mL or more, about 110 mL or more, about 120 mL or more, about 130 mL or more, about 140 mL or more, about 150 mL or more, about 160 mL or more, about 170 mL or more, about 180 mL or more, or about 185 mL or more, or between a range of any two of the foregoing values, over a period of about 12 weeks. The increase in forced vital capacity can be about 10 mL to about 30 mL, about 10 mL to about 50 mL, about 10 mL to about 75 mL, about 10 mL to about 100 mL, about 10 mL to about 125 mL, about 10 mL to about 150 mL, about 10 mL to about 175 mL, about 10 mL to about 185 mL, about 20 mL to about 40 mL, about 30 mL to about 50 mL, about 30 mL to about 75 mL, about 30 mL to about 100 mL, about 30 mL to about 125 mL, about 30 mL to about 150 mL, about 30 mL to about 175 mL, about 30 mL to about 185 mL, about 50 mL to about 75 mL, about 50 mL to about 100 mL, about 50 mL to about 125 mL, about 50 mL to about 150 mL, about 50 mL to about 175 mL, about 50 mL to about 185 mL, about 75 mL to about 100 mL, about 75 mL to about 125 mL, about 75 mL to about 150 mL, about 75 mL to about 175 mL, or about 75 mL to about 185 mL. The increase in forced vital capacity can be about 10 mL to about 30 mL, about 10 mL to about 50 mL, about 10 mL to about 75 mL, about 10 mL to about 100 mL, about 10 mL to about 125 mL, about 10 mL to about 150 mL, about 10 mL to about 175 mL, about 10 mL to about 185 mL, about 20 mL to about 40 mL, about 30 mL to about 50 mL, about 30 mL to about 75 mL, about 30 mL to about 100 mL, about 30 mL to about 125 mL, about 30 mL to about 150 mL, about 30 mL to about 175 mL, about 30 mL to about 185 mL, about 50 mL to about 75 mL, about 50 mL to about 100 mL, about 50 mL to about 125 mL, about 50 mL to about 150 mL, about 50 mL to about 175 mL, about 50 mL to about 185 mL, about 75 mL to about 100 mL, about 75 mL to about 125 mL, about 75 mL to about 150 mL, about 75 mL to about 175 mL, or about 75 mL to about 185 mL over a period of about 12 weeks.

The increase in forced vital capacity can be as compared to the increase in a subject who is not treated by administration of one or more of Compounds 1-780, such as Compound 5, or compared to an average increase in a group of subjects who are not treated by administration of one or more of Compounds 1-780, such as Compound 5. By way of illustration, if a subject who is administered one or more of Compounds 1-780 has an increase of forced vital capacity over a certain period, such as about 12 weeks, of about 30 mL, and a subject who is not administered one or more of Compounds 1-780 has an increase of forced vital capacity over the same period of about 5 mL, then the increase of forced vital capacity is 25 mL over the period. Alternatively, the increase of forced vital capacity at the end of a certain period of administration of one or more of Compounds 1-780, such as 12 weeks, to a subject, can be compared to the forced vital capacity of the subject at the start of the period.

### Kits

The invention further provides kits for carrying out the methods of the invention, which comprises one or more compounds described herein, or a salt thereof, or a pharmacological composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for use in the treatment of a fibrotic disease.

Kits generally comprise suitable packaging. The kits may comprise one or more containers comprising any compound described herein. Each component (if there is more than one component) can be packaged in separate containers or some components can be combined in one container where cross-reactivity and shelf life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a disease detailed herein (e.g., fibrosis) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The kits may optionally further comprise instructions for daily administration of the dosage form to an individual in need thereof, such as instructions for administration of the dosage form to an individual in need thereof one, two, three, or four times daily, for example, instructions for administration of the dosage form to an individual in need thereof once daily.

### General Procedures

Compounds provided herein may be prepared according to General Schemes, as exemplified by the General Procedures and Examples. Minor variations in temperatures, concentrations, reaction times, and other parameters can be made when following the General Procedures, which do not substantially affect the results of the procedures.

When a specific stereoisomer, or an unspecified stereoisomer, or a mixture of stereoisomers is shown in the following general procedures, it is understood that similar chemical transformations can be performed on other specific stereoisomers, or an unspecified stereoisomer, or mixtures thereof. For example, a hydrolysis reaction of a methyl (S)-4-amino-butanoate to an (S)-4-amino-butanoic acid can also be performed on a methyl (R)-4-amino-butanoate to prepare an (R)-4-amino-butanoic acid, or on a mixture of a methyl (S)-4-amino-butanoat and a methyl (R)-4-amino-butanoate to prepare a mixture of an (S)-4-amino-butanoic acid and an (R)-4-amino-butanoic acid.

Some of the following general procedures use specific compounds to illustrate a general reaction (e.g., deprotection of a compound having a Boc-protected amine to a compound having a deprotected amine using acid). The general reaction can be carried out on other specific compounds having the same functional group (e.g., a different compound having a protected amine where the Boc-protecting group can be removed using acid in the same manner) as long as such other specific compounds do not contain additional functional groups affected by the general reaction (i.e., such other specific compounds do not contain acid-sensitive functional groups), or if the effect of the general reaction on those additional functional groups is desired (e.g., such other specific compounds have another group that is affected by acid, and the effect of the acid on that other group is a desirable reaction).

Where specific reagents or solvents are specified for reactions in the general procedures, the skilled artisan will recognize that other reagents or solvents can be substituted as desired. For example, where hydrochloric acid is used to remove a Boc group, trifluoroacetic acid can be used instead. As another example, where HATU (1-[bis(dimethylamino)methylene]-1H-1,2,3-triazolo[4,5-b]pyridinium 3-oxide hexafluorophosphate) is used as a coupling reagent, BOP (benzotriazol-1 -y]oxytris(dimethylamino)phosphonium hexafluorophosphate) or PyBOP (benzotriazol-1 -yl-oxytripyrrolidinophosphonium hexafluorophosphate) can be used instead.

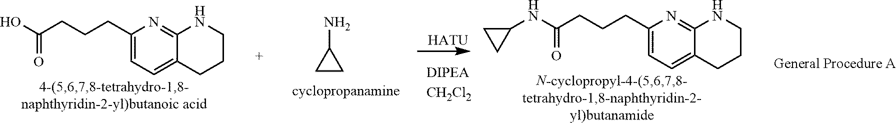

N-cyclopropyl-4-(5,6,7,8-tetrahydro-1,8-naphthyridin-2-yl)butanamide. To a mixture of 4-(5,6,7,8-tetrahydro-1,8-naphthyridin-2-yl)butanoic acid hydrochloride (5.0 g, 19.48 mmol) and cyclopropanamine (1.51 mL, 21.42 mmol) in CH₂Cl₂ (80 mL) at rt was added DIPEA (13.57 mL, 77.9 mmol). To this was then added HATU (8.1 g, 21.42 mmol) and the resulting mixture was stirred at rt for 2 hrs. The reaction mixture was concentrated in vacuo and purified by normal phase silica gel chromatography to give N-cyclopropyl-4-(5,6,7,8-tetrahydro-1,8-naphthyridin-2-yl)butanamide.

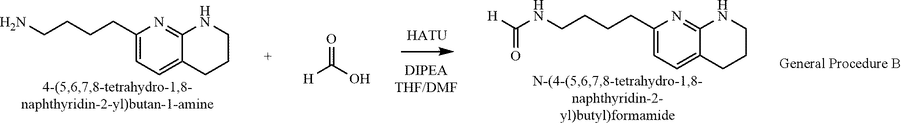

N-(4-(5,6,7,8-tetrahydro-1,8-naphthyridin-2-yl)butyl)formamide. To a mixture of 4-(5,6,7,8-tetrahydro-1,8-naphthyridin-2-yl)butan-1-amine (351 mg, 1.71 mmol) and formic acid (0.09 mL, 2.22 mmol) in 4:1 THF/DMF (5 mL) was added HATU (844 mg, 2.22 mmol) followed by DIPEA (0.89 mL, 5.13 mmol) and the reaction was allowed to stir at rt for 1 hr. The reaction mixture was concentrated in vacuo and purified by normal phase silica gel chromatography to give N-(4-(5,6,7,8-tetrahydro-1,8-naphthyridin-2-yl)butyl)formamide.

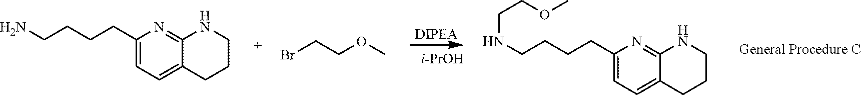

N-(2-methoxyethyl)-4-(5,6,7,8-tetrahydro-1,8-naphthyridin-2-yl)butan-1-amine. A mixture of 4-(5,6,7,8-tetrahydro-1,8-naphthyridin-2-yl)butan-1-amine (300 mg, 1.46 mmol), 1-bromo-2-methoxyethane (0.11 mL, 1.17 mmol) and DIPEA (0.25 mL, 1.46 mmol) in i-PrOH (3 mL) was heated to 70° C. for 18 hr. The reaction mixture was allowed to cool to rt and then concentrated in vacuo and purified by normal phase silica gel chromatography to give N-(2-methoxyethyl)-4-(5,6,7,8-tetrahydro-1,8-naphthyridin-2-yl)butan-1-amine.

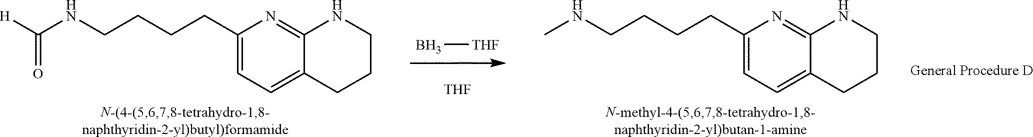

N-methyl-4-(5,6,7,8-tetrahydro-1,8-naphthyridin-2-yl)butan-1-amine. To a solution of N-(4-(5,6,7,8-tetrahydro-1,8-naphthyridin-2-yl)butyl)formamide (200 mg, 0.86 mmol) in THF (2 mL) at rt was added borane tetrahydrofuran complex solution (1.0 M in THF, 4.0 mL, 4.0 mmol) dropwise. The resulting mixture was then heated to 60° C. for 2 hr and then allowed to cool to rt. The reaction mixture was diluted with MeOH and concentrated in vacuo. The crude residue was purified by normal phase silica gel chromatography to give N-methyl-4-(5,6,7,8-tetrahydro-1,8-naphthyridin-2-yl)butan-1-amine.

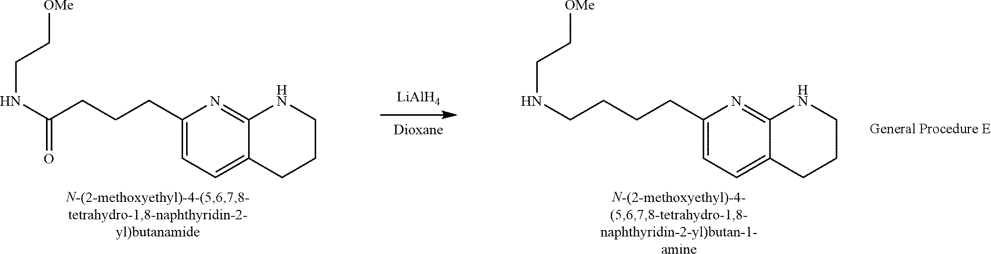

N-(2-methoxyethyl)-4-(5,6,7,8-tetrahydro-1,8-naphthyridin-2-yl)butan-1-amine (5). To a solution of N-(2-methoxyethyl)-4-(5,6,7,8-tetrahydro-1,8-naphthyridin-2-yl)butanamide (15.5 g, 1.0 equiv) in 1,4-dioxane (124 mL) at rt was slowly added LiA1H₄ (1.0 M in THF, 123 mL, 2.2 equiv) and the resulting mixture was heated to reflux for 20 hours and then cooled to 0° C. To this solution was added H₂O (4.7 mL), then 1 M NaOH (4.7 mL) then H₂O (4.7 mL) and warmed to room temperature and stirred for 30 minutes, at which time, solid MgSO₄ was added and stirred for an additional 30 minutes. The resulting mixture was filtered and the filter cake was washed with THF. The filtrate were concentrated in vacuo to give N-(2-methoxyethyl)-4-(5,6,7,8-tetrahydro-1,8-naphthyridin-2-yl)butan-1-amine.

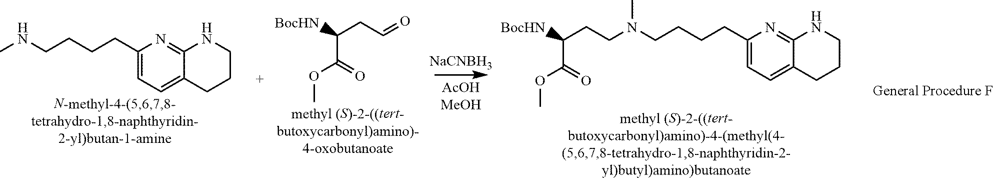

methyl (S)-2-((tert-butoxycarbonyl)amino)-4-(methyl(4-(5,6,7,8-tetrahydro-1,8-naphthyridin-2-yl)butyl)amino)butanoate. To a mixture of N-methyl-4-(5,6,7,8-tetrahydro-1,8-naphthyridin-2-yl)butan-1-amine (5) (187 mg, 0.85 mmol) in MeOH (5 mL) at rt was added acetic acid (0.12 mL, 2.05 mmol) followed by methyl (S)-2-((tert-butoxycarbonyl)amino)-4-oxobutanoate (217 mg, 0.94 mmol). The resulting mixture was allowed to stir at rt for 15 min, at which time, sodium cyanoborohydride (80 mg, 1.28 mmol) was added to the reaction mixture and stirred for 30 min and then concentrated in vacuo. The crude residue was purified by normal phase silica gel chromatography to give methyl (S)-2-((tert-butoxycarbonyl)amino)-4-(methyl(4-(5,6,7,8-tetrahydro-1,8-naphthyridin-2-yl)butyl)amino)butanoate.

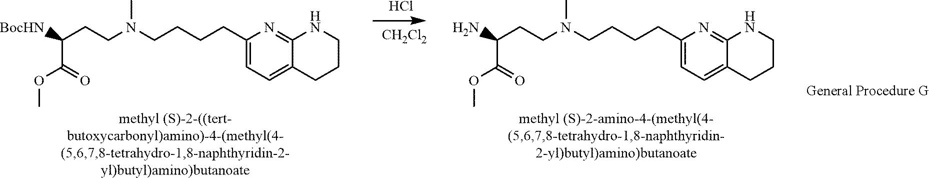

methyl (S)-2-amino-4-(methyl(4-(5,6,7,8-tetrahydro-1,8-naphthyridin-2-yl)butyl)amino)butanoate. To a solution of methyl (S)-2-((tert-butoxycarbonyl)amino)-4-(methyl(4-(5,6,7,8-tetrahydro-1,8-naphthyridin-2-yl)butyl)amino)butanoate (152 mg, 0.35 mmol) in CH₂C1₂ (2 mL) at rt was added 4N HC1 in 1,4-dioxane (1 mL, 4 mmol) and the resulting mixture was allowed to stir for 2 hr. The reaction mixture was concentrated in vacuo to give methyl (S)-2-amino-4-(methyl(4-(5,6,7,8-tetrahydro-1,8-naphthyridin-2-yl)butyl)amino)butanoate as the trihydrochloride salt.

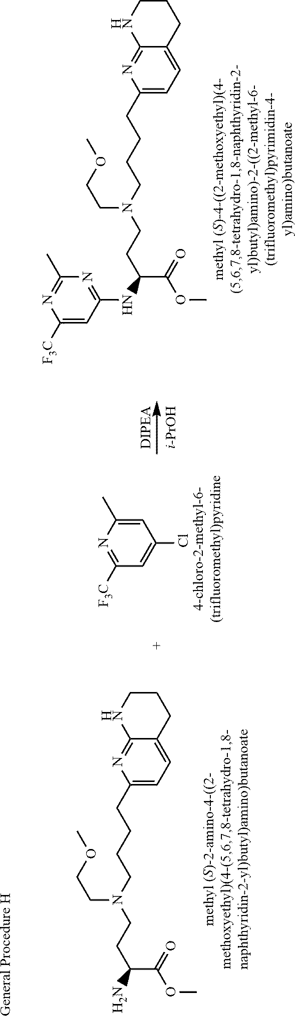

A solution of methyl (S)-2-amino-4-((2-methoxyethyl)(4-(5,6,7,8-tetrahydro-1,8-naphthyridin-2-yl)butyl)amino)butanoate trihydrochloride (80 mg, 0.16 mmol), 4-chloro-2-methyl-6-(trifluoromethyl)pyrimidine (64 mg, 0.33 mmol) and DIPEA (0.23 mL, 1.31 mmol) in i-PrOH (1 mL) was heated at 60° C. overnight. The reaction was allowed to cool to rt and then concentrated in vacuo. The resulting crude residue was purified by normal phase silica gel chromatography to give methyl (S)-4-((2-methoxyethyl)(4-(5,6,7,8-tetrahydro-1,8-naphthyridin-2-yl)butyl)amino)-2-((2-methyl-6-(trifluoromethyl)pyrimidin-4-yl)amino)butanoate.

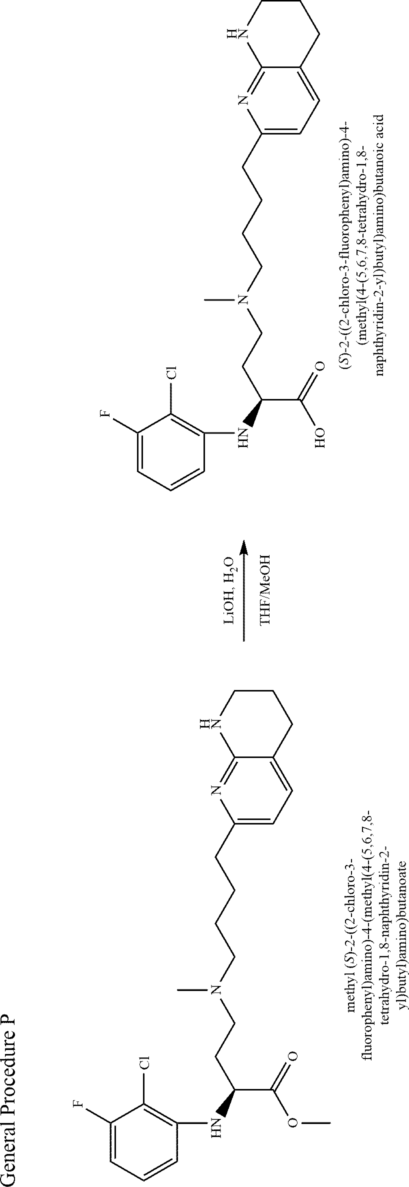

(S)-2-((2-chloro-3-fluorophenyl)amino)-4-(methyl(4-(5,6,7,8-tetrahydro-1,8-naphthyridin-2-yl)butyl)amino)butanoic acid To a solution of methyl (S)-2-((2-chloro-3-fluorophenyl)amino)-4-(methyl(4-(5,6,7,8-tetrahydro-1,8-naphthyridin-2-yl)butyl)amino)butanoate in 4:1:1 THF/MeOH/H₂O at rt was added lithium hydroxide (approximately four equivalents) and the resulting mixture was stirred for 30 min. The reaction mixture was concentrated in vacuo and the resulting crude residue purified by reverse phase HPLC to give (S)-2-((2-chloro-3-fluorophenyl)amino)-4-(methyl(4-(5,6,7,8-tetrahydro-1,8-naphthyridin-2-yl)butyl)amino)butanoic acid, as the trifluoroacetate salt.

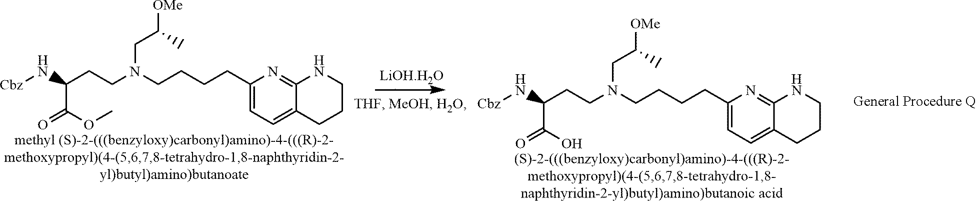

(S)-2-(((benzyloxy)carbonyl)amino)-4-(((R)-2-methoxypropyl)(4-(5,6,7,8-tetrahydro-1,8-naphthyridin-2-yl)butyl)amino)butanoic acid. A mixture of methyl (S)-2-(((benzyloxy)carbonyl)amino)-4-(((R)-2-methoxypropyl)(4-(5,6,7,8-tetrahydro-1,8-naphthyridin-2-yl)butyl)amino)butanoate (1 g, 1.90 mmol) in H₂O (3 mL) and THF (3 mL) and MeOH (3 mL) was added LiOH•H₂O (159.36 mg, 3.80 mmol) and then the mixture was stirred at room temperature for 1 h and the resulting mixture was concentrated in vacuo. The mixture was adjusted to pH=6 by AcOH (2 mL) and the residue was concentrated in vacuo to give a residue to yield compound (S)-2-(((benzyloxy)carbonyl)amino)-4-(((R)-2-methoxypropyl)(4-(5,6,7,8-tetrahydro-1,8-naphthyridin-2-yl)butyl)amino)butanoic acid. LCMS (ESI+): m/z = 513.5 (M+H)⁺. ¹H NMR (400 MHz, DMSO-d): δ ppm 7.25 - 7.37 (m, 5 H) 7.00 (d, J=7.28 Hz, 1 H) 6.81 (br d, J=7.50 Hz, 1 H) 6.22 (d, J=7.28 Hz, 1 H₆) 4.93 - 5.05 (m, 2 H) 3.68 - 3.77 (m, 1 H) 3.25 - 3.34 (m, 1 H) 3.15 - 3.24 (m, 5 H) 2.58 (br t, J=6.06 Hz, 2 H) 2.29 - 2.49 (m, 8 H) 2.16 (br dd, J=12.90, 6.06 Hz, 1 H) 1.69 - 1.78 (m, 2 H) 1.58 - 1.68 (m, 1 H) 1.53 (quin, J=7.39 Hz, 2 H) 1.28 - 1.40 (m, 2 H) 1.00 (d, J=5.95 Hz, 3 H).

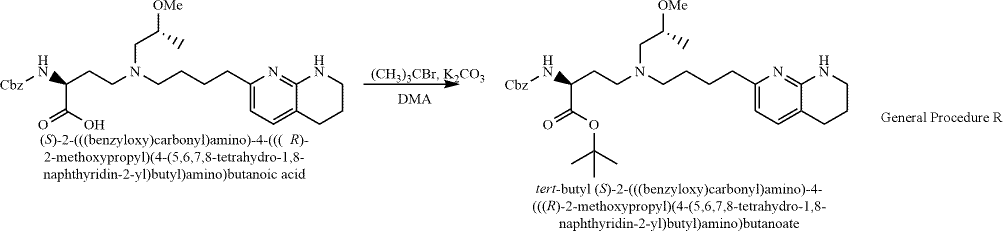

tert-butyl (S)-2-(((benzyloxy)carbonyl)amino)-4-(((R)-2-methoxypropyl)(4-(5,6,7,8-tetrahydro-1,8-naphthyridin-2-yl)butyl)amino)butanoate: A solution of (S)-2-(((benzyloxy)carbonyl)amino)-4-(((R)-2-methoxypropyl)(4-(5,6,7,8-tetrahydro-1,8-naphthyridin-2-yl)butyl)amino)butanoic acid (300 mg, 523.84 umol, HOAc salt) in DMA (4 mL) was added N-benzyl-N,N-diethylethanaminium chloride (119.32 mg, 523.84 umol), K₂CO₃ (1.88 g, 13.62 mmol), 2-bromo-2-methylpropane (3.45 g, 25.14 mmol,). The mixture was stirred for 18 h at the 55° C. and then allowed to cool to room temperature. The reaction mixture was concentrated in vacuo and the aqueous phase was extracted with ethyl acetate. The combined organic extracts were washed with brine, dried over Na₂SO₄, filtered, and concentrated in vacuo. The crude residue was purified by prep-TLC to give tert-butyl (S)-2-(((benzyloxy)carbonyl)amino)-4-(((R)-2-methoxypropyl)(4-(5,6,7,8-tetrahydro-1,8-naphthyridin-2-yl)butyl)amino)butanoate. LCMS (ESI+): m/z = 569.3 (M+H)⁺.

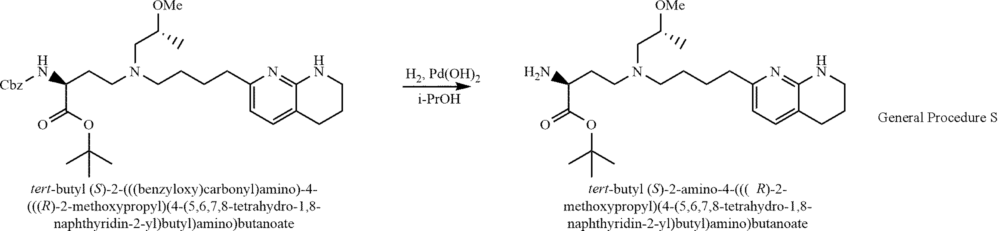

tert-buₜyl (S)-2-amino-4-(((R)-2-methoxypropyl)(4-(5,6,7,8-tetrahydro-1,8-naphthyridin-2-yl)butyl)amino)butanoate. To a solution of tert-butyl (S)-2-(((benzyloxy)carbonyl)amino)-4-(((R)-2-methoxypropyl)(4-(5,6,7,8-tetrahydro-1,8-naphthyridin-2-yl)butyl)amino)butanoate (107 mg, 188.13 umol) in i-PrOH (2 mL) was added Pd(OH)₂ (26 mg) under an N₂ atmosphere. The suspension was degassed under vacuum and purged with H₂ several times. The mixture was stirred under H₂ (15 psi) at room temperature for 15 h. The mixture was filtered and concentrated in vacuo to give tert-butyl (S)-2-amino-4-(((R)-2-methoxypropyl)(4-(5,6,7,8-tetrahydro-1,8-naphthyridin-2-yl)butyl)amino)butanoate. LCMS (ESI+): m/z = 435.5 (M+H)⁺. ¹HNMR (400 MHz, CDC1₃): δ ppm 7.06 (d, J=7.34 Hz, 1 H) 6.34 (d, J=7.34 Hz, 1 H) 4.98 (br s, 1 H) 3.38 - 3.44 (m, 4 H) 3.34 (s, 3 H) 2.69 (t, J=6.30 Hz, 2 H) 2.51 - 2.59 (m, 5 H) 2.31 (dd, J=13.39, 5.56 Hz, 1 H) 1.86 - 1.94 (m, 5 H) 1.49 - 1.69 (m, 6 H) 1.47 (s, 9 H) 1.13 (d, J=6.11 Hz, 3 H).

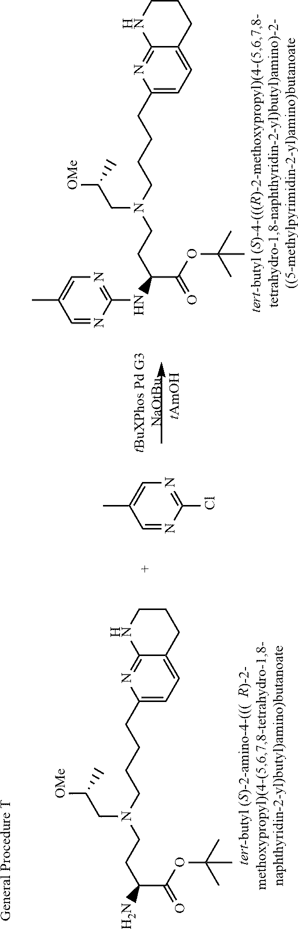

tert-butyl (S)-4-(((R)-2-methoxypropyl)(4-(5,6,7,8-tetrahydro-1,8-naphthyridin-2-yl)butyl)amino)-2-((5-methylpyrimidin-2-yl)amino)butanoate. To a solution of (S)-tert-butyl 2-amino-4-(((R)-2-methoxypropyl)(4-(5,6,7,8-tetrahydro-1,8-naphthyridin-2-yl)butyl)amino)butanoate tert-butyl (S)-2-amino-4-(((R)-2-methoxypropyl)(4-(5,6,7,8-tetrahydro-1,8-naphthyridin-2-yl)butyl)amino)butanoate (100 mg, 230.09 umol) and 2-chloro-5-methyl-pyrimidine (24.65 mg, 191.74 umol) in 2-methyl-2-butanol (2 mL) was added t-BuONa (2 M in THF, 191.74 uL) and [2-(2-aminophenyl)phenyl]-methylsulfonyloxy-palladium;ditert-butyl-[2-(2,4,6-triisopropylphenyl)phenyl]phosphane (15.23 mg, 19.17 umol), and the resulting mixture was stirred at 100° C. for 14 h. The mixture was concentrated in vacuo to give (S)-tert-butyl 4-(((S)-2-methoxypropyl)(4-(5,6,7,8-tetrahydro-1,8-naphthyridin-2-yl)butyl)amino)-2-((5-methylpyrimidin-2-yl)amino)butanoate. LCMS (ESI+): m/z = 527.3 (M+H)⁺.

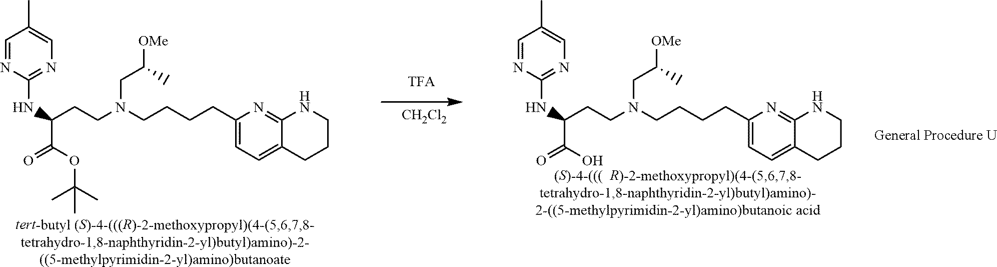

(S)-4-(((R)-2-methoxypropyl)(4-(5,6,7,8-tetrahydro-1,8-naphthyridin-2-yl)butyl)amino)-2-((5-methylpyrimidin-2-yl)amino)butanoic acid. To a solution of tert-butyl (S)-4-(((R)-2-methoxypropyl)(4-(5,6,7,8-tetrahydro-1,8-naphthyridin-2-yl)butyl)amino)-2-((5-methylpyrimidin-2-yl)amino)butanoate (80 mg, 151.89 umol) in DCM (2 mL) was added TFA (254.14 mg, 2.23 mmol) at 0° C. The mixture was stirred at room temperature for 6 h. The mixture was concentrated in vacuo and the resulting crude residue was purified by prep-HPLC to give compound (S)-4-(((R)-2-methoxypropyl)(4-(5,6,7,8-tetrahydro-1,8-naphthyridin-2-yl)butyl)amino)-2-((5-methylpyrimidin-2-yl)amino)butanoic acid. LCMS (ESI+): m/z = 471.2 (M+H)⁺. ¹H NMR (400 MHz, Methanol-d₄) δ ppm 8.57 (br s, 2 H) 7.60 (d, J=7.28 Hz, 1 H) 6.67 (d, J=7.28 Hz, 1 H) 4.81 - 4.86 (m, 1 H) 3.86 (br s, 1 H) 3.41 - 3.59 (m, 4 H) 3.39 (s, 3 H) 3.33 - 3.38 (m, 1 H) 3.12 - 3.30 (m, 3 H) 2.76 - 2.86 (m, 4 H) 2.54 (br s, 1 H) 2.39 (br d, J=8.82 Hz, 1 H) 2.30 (s, 3 H) 1.76 - 1.99 (m, 6 H) 1.22 (d, J=5.95 Hz, 3 H).

### Enumerated Embodiments

The following enumerated embodiments are representative of some aspects of the invention.

Embodiment 1. A method of treating a subject for a disease, comprising: administering to the subject a first drug comprising a compound of formula (A):

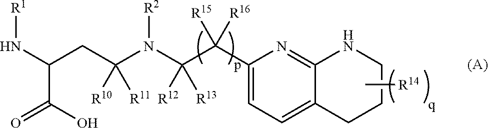

or a salt thereof; and
administering to the subject at least a second drug that is selected from the group consisting of: pirfenidone and nintedanib, or a salt thereof, whereby the subject is treated for the disease;
wherein in the compound of Formula (A):
   R¹ is C₆-C₁₄ aryl or 5- to 10-membered heteroaryl wherein the C₆-C₁₄ aryl and 5- to 10-membered heteroaryl are optionally substituted by R^{1a};
   R² is hydrogen; deuterium; C₁-C₆ alkyl optionally substituted by R^{2a}; —OH; —O—C₁—C₆ alkyl optionally substituted by R^{2a}; C₃-C₆ cycloalkyl optionally substituted by
   R^{2b}; —O—C₃—C₆ cycloalkyl optionally substituted by R^{2b}; 3- to 12-membered heterocyclyl optionally substituted by R^{2c}; or -S(O)₂R^{2d}; with the proviso that any carbon atom bonded directly to a nitrogen atom is optionally substituted with an R^{2a} moiety other than halogen; each R^{1a} is independently C₁-C₆ alkyl, C₂-C₆ alkenyl, C₂-C₆ alkynyl, C₃-C₈ cycloalkyl, C₄-Cs cycloalkenyl, 3- to 12-membered heterocyclyl, 5- to 10-membered heteroaryl, C₆-C₁₄ aryl, deuterium, halogen, —CN, -OR³, —SR³, -NR⁴R⁵, —NO₂, -C=NH(OR³), -C(O)R³, -OC(O)R³, -C(O)OR³, -C(O)NR⁴R⁵, -NR³C(O)R⁴, -NR³C(O)OR⁴, -NR³C(O)NR⁴R⁵, -S(O)R³, -S(O)₂R³,
   -NR³S(O)R⁴, -NR³S(O)₂R⁴, -S(O)NR⁴R⁵, -S(O)₂NR⁴R⁵, or -P(O)(OR⁴)(OR⁵), wherein each R^{1a} is, where possible, independently optionally substituted by deuterium, halogen, oxo,
   -OR⁶, -NR⁶R⁷, -C(O)R⁶, —CN, -S(O)R⁶, -S(O)₂R⁶, -P(O)(OR⁶)(OR⁷), C₃-C₈ cycloalkyl, 3- to 12-membered heterocyclyl, 5- to 10-membered heteroaryl, C₆-C₁₄ aryl, or C₁-C₆ alkyl optionally substituted by deuterium, oxo, —OH or halogen;
   each R^{2a}, R^{2b}, R^{2c}, R^{2e}, and R^{2f} is independently oxo or R^{1a};
   R^{2d} is C₁-C₆ alkyl optionally substituted by R^{2e} or C₃-C₅ cycloalkyl optionally substituted by R^{2f.,}
   R³ is independently hydrogen, deuterium, C₁-C₆ alkyl, C₂-C₆ alkenyl, C₂-C₆ alkynyl, C₃-C₆ cycloalkyl, C₆-C₁₄ aryl, 5- to 10-membered heteroaryl or 3- to 12-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 10-membered heteroaryl and 3- to 12-membered heterocyclyl of R³ are independently optionally substituted by halogen, deuterium, oxo, —CN, -OR⁸, -NR⁸R⁹, -P(O)(OR⁸)(OR⁹), or
   C₁-C₆ alkyl optionally substituted by deuterium, halogen, —OH or oxo;
   R⁴ and R⁵ are each independently hydrogen, deuterium, C₁-C₆ alkyl, C₂-C₆ alkenyl, C₂-C₆ alkynyl, C₃-C₆ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 6-membered heteroaryl and 3- to 6-membered heterocyclyl of R⁴ and R⁵ are independently optionally substituted by deuterium, halogen, oxo, —CN, -OR, -NR⁸R⁹ or C₁-C₆ alkyl optionally substituted by deuterium, halogen, —OH or oxo;
   or R⁴ and R⁵ are taken together with the atom to which they attached to form a 3- to 6-membered heterocyclyl optionally substituted by deuterium, halogen, oxo, -OR⁸, -NR⁸R⁹ or
   C₁-C₆ alkyl optionally substituted by deuterium, halogen, oxo or —OH;
   R⁶ and R⁷ are each independently hydrogen, deuterium, C₁-C₆ alkyl optionally substituted by deuterium, halogen, or oxo, C₂-C₆ alkenyl optionally substituted by deuterium, halogen, or
   oxo, or C₂-C₆ alkynyl optionally substituted by deuterium, halogen, or oxo;
   or R⁶ and R⁷ are taken together with the atom to which they attached to form a 3- to 6-membered heterocyclyl optionally substituted by deuterium, halogen, oxo or C₁-C₆ alkyl optionally substituted by deuterium, halogen, or oxo;
   R⁸and R⁹ are each independently hydrogen, deuterium, C₁-C₆ alkyl optionally substituted by deuterium, halogen, or oxo, C₂-C₆ alkenyl optionally substituted by deuterium, halogen or
   oxo, or C₂-C₆ alkynyl optionally substituted by deuterium, halogen, or oxo;
   or R⁸ and R⁹ are taken together with the atom to which they attached to form a 3-6 membered heterocyclyl optionally substituted by deuterium, halogen, oxo or C₁-C₆ alkyl optionally substituted by deuterium, oxo, or halogen;
   each R¹⁰, R¹¹, R¹² and R¹³ are independently hydrogen or deuterium;
   R¹⁴ is deuterium;
   q is 0, 1, 2, 3, 4, 5, 6, 7, or 8;
   each R¹⁵ is independently selected from hydrogen, deuterium, or halogen;
   each R¹⁶ is independently selected from hydrogen, deuterium, or halogen; and p is 3, 4, 5, 6, 7, 8, or 9.

Embodiment 2. The method of Embodiment 1, wherein in the compound of Formula (A) or a salt thereof:
R² is C₁-C₆ alkyl optionally substituted by R^{2a}; C₃-C₆ cycloalkyl optionally substituted by R^{2b}; 3- to 12-membered heterocyclyl optionally substituted by R^{2c}; or -S(O)₂R^{2d}; R³ is independently hydrogen, deuterium, C₁-C₆ alkyl, C₂-C₆ alkenyl, C₂-C₆ alkynyl, C₃-C₆ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 6-membered heteroaryl and 3- to 6-membered heterocyclyl of R³ are independently optionally substituted by halogen, deuterium, oxo, —CN, -OR⁸, -NR⁸R⁹, -P(O)(OR⁸)(OR⁹), or C₁-C₆ alkyl
optionally substituted by deuterium, halogen, —OH or oxo;
each R¹⁵ is hydrogen; and
each R¹⁶ is hydrogen;
wherein the compound of Formula (A) is represented by Formula (I):

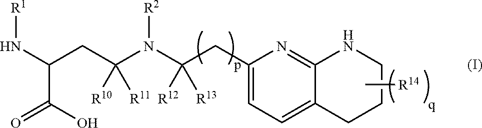

or a salt thereof.

Embodiment 3. The method of Embodiment 1 or Embodiment 2, wherein in the compound of Formula (A) or (I) or a salt thereof, at least one of R^{1a}, R^{2a}, R^{2b}, R^{2c}, R^{2e}, R^{2f}, R³, R⁴, R⁵, R⁶, R⁷, R⁸, R⁹, R¹⁰, R¹¹, R¹², R¹³, or R¹⁴ is deuterium.

Embodiment 4. The method of Embodiment 1 or Embodiment 2, wherein in the compound of Formula (A) or (I) or a salt thereof, R¹⁰, R¹¹, R¹², R¹³, and R¹⁴ are hydrogen; p is 3; and wherein the compound of Formula (A) or (I) is represented by the compound of formula (II):

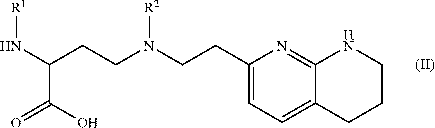

Embodiment 5. The method of any one of Embodiments 1-4, wherein in the compound of Formula (A) or (I) or a salt thereof, R¹ is 5- to 10-membered heteroaryl optionally substituted by R^{1a}.

Embodiment 6. The method of any one of Embodiments 1-4, wherein in the compound of Formula (A), (I), or (II), or a salt thereof, R¹ is:
pyrimidinyl, quinazolinyl, pyrazolopyrimidinyl, pyrazinyl, quinolinyl, pyridopyrimidinyl, thienopyrimidinyl, pyridinyl, pyrrolopyrimidinyl, quinoxalinyl, indazolyl, benzothiazolyl, naphthalenyl, purinyl, or isoquinolinyl; and
optionally substituted by deuterium, hydroxy, C₁-C₆ alkyl, C₁-C₆ haloalkyl, C₁-C₆ perhaloalkyl, C₁-C₆ alkoxyl, C₃-C₈ cycloalkyl, C₃-C₈ halocycloalkyl, C₃-C₈ cycloalkoxyl, 5-to 10-membered heteroaryl, C₆-C₁₄ aryl, cyano, amino, alkylamino, or dialkylamino.

Embodiment 7. The method of any one of Embodiments 1-4, wherein in the compound of Formula (A), (I), or (II), or a salt thereof, R¹ is:
pyrimidin-2-yl, pyrimidin-4-yl, quinazolin-4-yl, 1H-pyrazolo[3,4-d]pyrimidine-4-yl, 1H-pyrazolo[4,3-d]pyrimidine-7-yl, pyrazin-2-yl, quinoline-4-yl, pyrido[2,3-d]pyrimidin-4-yl, pyrido[3,2-d]pyrimidin-4-yl, pyrido[3,4-d]pyrimidin-4-yl, thieno[2,3-d]pyrimidin-4-yl, thieno[3,2-d]pyrimidin-4-yl, thienopyrimidin-4-yl, pyridin-2-yl, pyridin-3-yl, 7H-pyrrolo[2,3-d]pyrimidin-4-yl, quinoxalin-2-yl, 1H-indazol-3-yl, benzo[d]thiazol-2-yl, naphthalen-1-yl, 9H-purin-6-yl, or isoquinolin-1-yl; and
optionally substituted by: one or more deuterium; methyl; cyclopropyl; fluoro; chloro; bromo; difluoromethyl; trifluoromethyl; methyl and fluoro; methyl and trifluoromethyl; methoxy; cyano; dimethylamino; phenyl; pyridin-3-yl; or pyridin-4-yl.

Embodiment 8. The method of any one of Embodiments 1-4, wherein in the compound of Formula (A), (I), or (II), or a salt thereof, R¹ is pyrimidin-4-yl optionally substituted by R^{1a}.

Embodiment 9. The method of any one of Embodiments 1-4, wherein in the compound of Formula (A), (I), or (II), or a salt thereof, R¹ is pyrimidin-4-yl optionally substituted by R^{1a} wherein R^{1a} is 5- to 10-membered heteroaryl or C₁-C₆ alkyl optionally substituted by halogen.

Embodiment 10. The method of any one of Embodiments 1-4, wherein in the compound of Formula (A), (I), or (II), or a salt thereof, R¹ is pyrimidin-4-yl optionally substituted by pyrazolyl, methyl, difluoromethyl, or trifluoromethyl.

Embodiment 11. The method of any one of Embodiments 1-4, wherein in the compound of Formula (A), (I), or (II), or a salt thereof, R¹ is pyrimidin-4-yl substituted by both methyl and trifluoromethyl.

Embodiment 12. The method of any one of Embodiments 1-4, wherein in the compound of Formula (A), (I), or (II), or a salt thereof, R¹ is quinazolin-4-yl optionally substituted by R^{1a}.

Embodiment 13. The method of any one of Embodiments 1-4, wherein in the compound of Formula (A), (I), or (II), or a salt thereof, R¹ is quinazolin-4-yl optionally substituted by halogen, C₁-C₆ alkyl optionally substituted by halogen, or C₁-C₆ alkoxy.

Embodiment 14. The method of any one of Embodiments 1-4, wherein in the compound of Formula (A), (I), or (II), or a salt thereof, R¹ is quinazolin-4-yl optionally substituted by fluoro, chloro, methyl, trifluoromethyl or methoxy.

Embodiment 15. The method of any one of Embodiments 1 or 3-14, wherein in the compound of Formula (A), (I), or (II), or a salt thereof, R² is:
hydrogen;
deuterium;
hydroxy; or
C₁-C₆ alkyl or C₁-C₆ alkoxyl optionally substituted with: deuterium, halogen, C₁-C₆ alkyl, C₁-C₆ haloalkyl, C₁-C₆ hydroxyalkyl, C₁-C₆ alkoxyl, C₃-C₈ cycloalkyl, C₃-C₈ halocycloalkyl, C₃-C₈ cycloalkoxyl, C₆-C₁₄ aryl, C₆-C₁₄ aryloxy, 5- to 10-membered heteroaryl, 5- to 10-membered heteroaryloxy, 3- to 12-membered heterocyclyl optionally substituted with oxo, -C(O)NR⁴R⁵, -NR³C(O)R⁴, or -S(O)₂R³.

Embodiment 16. The method of any one of Embodiments 1 or 3-14, wherein in the compound of Formula (A), (I), or (II), or a salt thereof, R² is:
methyl, methoxy, ethyl, ethoxy, propyl, cyclopropyl, or cyclobutyl;
each of which is optionally substituted with one or more of: hydroxy, methoxy, ethoxy, acetamide, fluoro, fluoroalkyl, phenoxy, dimethylamide, methylsulfonyl, cyclopropoxyl, pyridin-2-yloxy, optionally methylated or fluorinated pyridine-3-yloxy, N-morpholinyl, N-pyrrolidin-2-onyl, dimethylpyrazol-1-yl, dioxiran-2-yl, morpholin-2-yl, oxetan-3-yl, phenyl, tetrahydrofuran-2-yl, thiazol-2-yl; that is
each of which is substituted with 0, 1, 2, or 3 of deuterium, hydroxy, methyl, fluoro, cyano, or oxo.

Embodiment 17. The method of any one of Embodiments 1-14, wherein in the compound of Formula (A), (I), or (II), or a salt thereof, R² is C₁-C₆ alkyl optionally substituted by R^{2a}.

Embodiment 18. The method of any one of Embodiments 1-14, wherein in the compound of Formula (A), (I), or (II), or a salt thereof, R² is C₁-C₆ alkyl optionally substituted by R^{2a} wherein R^{2a} is: halogen; C₃-C₈ cycloalkyl optionally substituted by halogen; 5- to 10-membered heteroaryl optionally substituted by C₁-C₆ alkyl; -NR⁴R⁵; -NR³C(O)R⁴; -S(O)₂R³; or oxo.

Embodiment 19. The method of any one of Embodiments 1-14, wherein in the compound of Formula (A), (I), or (II), or a salt thereof, R² is C₁-C₆ alkyl optionally substituted by R^{2a} wherein R^{2a} is: fluoro; cyclobutyl substituted by fluoro; pyrazolyl substituted by methyl; or —S(O)₂CH₃.

Embodiment 20. The method of any one of Embodiments 1-14, wherein in the compound of Formula (A), (I), or (II), or a salt thereof, R² is C₁-C₆ alkyl optionally substituted by -OR³.

Embodiment 21. The method of any one of Embodiments 1-14, wherein in the compound of Formula (A), (I), or (II), or a salt thereof, R² is C₁-C₆ alkyl optionally substituted by -OR³, and R³ is: hydrogen; C₁-C₆ alkyl optionally substituted by halogen; C₃-C₆ cycloalkyl optionally substituted by halogen; C₆-C₁₄ aryl optionally substituted by halogen; or 5- to 6-membered heteroaryl optionally substituted by halogen or C₁-C₆ alkyl.

Embodiment 22. The method of any one of Embodiments 1-14, wherein in the compound of Formula (A), (I), or (II), or a salt thereof, R² is C₁-C₆ alkyl optionally substituted by -OR³, and R³ is: hydrogen; methyl; ethyl; difluoromethyl; —CH₂CHF₂; —CH₂CF₃; cyclopropyl substituted by fluoro; phenyl optionally substituted by fluoro; or pyridinyl optionally substituted by fluoro or methyl.

Embodiment 23. The method of any one of Embodiments 1 to 14, wherein in the compound of Formula (A), (I), or (II), or a salt thereof, R² is —CH₂CH₂OCH₃.

Embodiment 24. The method of any one of Embodiments 1 to 14, wherein in the compound of Formula (A), (I), or (II), or a salt thereof, R² is C₁-C₆ alkyl substituted by both halogen and OR³, wherein R³ is C₁-C₆ alkyl.

Embodiment 25. The method of any one of Embodiments 1 to 14, wherein in the compound of Formula (A), (I), or (II), or a salt thereof, R² is C₃-C₆ cycloalkyl optionally substituted by R^{2b}.

Embodiment 26. The method of any one of Embodiments 1 to 14, wherein in the compound of Formula (A), (I), or (II), or a salt thereof, R² is cyclopropyl.

Embodiment 27. The method of any one of Embodiments 1-4, wherein in the compound of Formula (A), (I), or (II), or a salt thereof, R¹ is

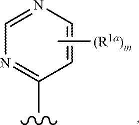

wherein m is 0, 1,2, or 3 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 28. The method of Embodiment 27, wherein in the compound of Formula (A), (I), or (II), or a salt thereof, R¹ is

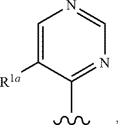

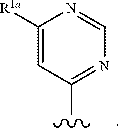

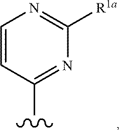

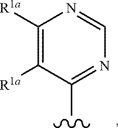

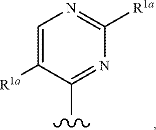

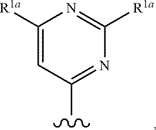

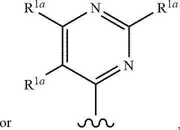

wherein each R^{1a} is independently deuterium, alkyl, haloalkyl, or heteroaryl.

Embodiment 29. The method of any one of Embodiments 1-4, wherein in the compound of Formula (A), (I), or (II), or a salt thereof, R¹ is

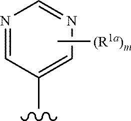

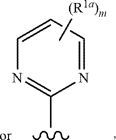

wherein m is 0, 1, 2, or 3 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 30. The method of any one of Embodiments 1-4, wherein in the compound of Formula (A), (I), or (II), or a salt thereof, R¹ is

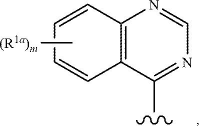

wherein m is 0, 1, 2, 3, 4, or 5 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 31. The method of Embodiment 30, wherein in the compound of Formula (A), (I), or (II), or a salt thereof, R¹ is

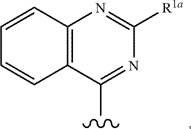

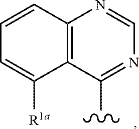

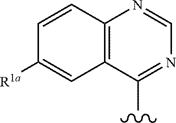

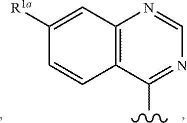

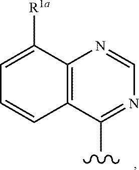

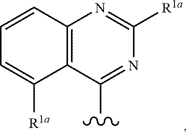

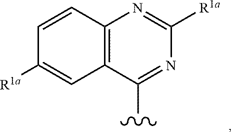

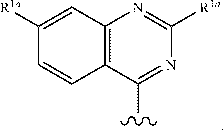

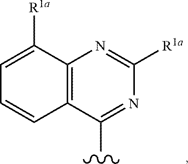

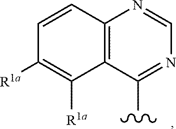

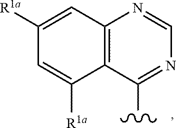

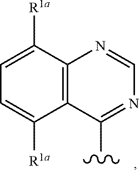

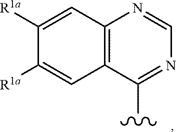

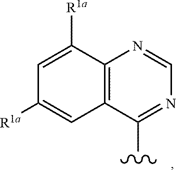

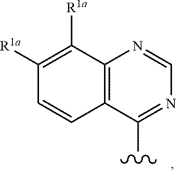

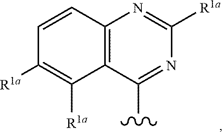

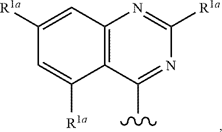

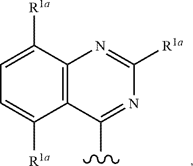

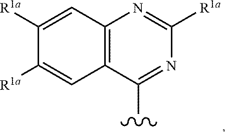

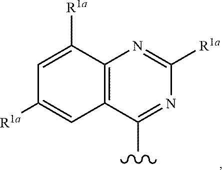

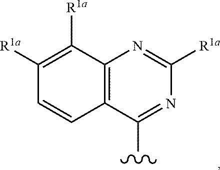

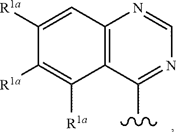

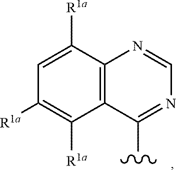

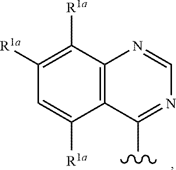

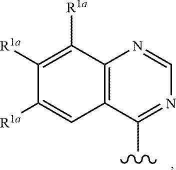

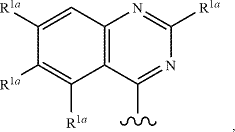

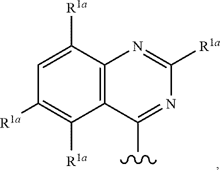

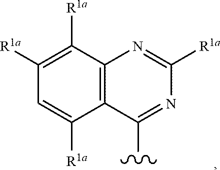

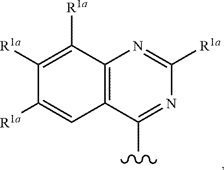

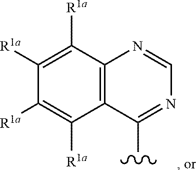

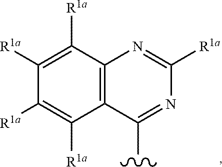

wherein each R^{1a} is independently deuterium, halogen, alkyl, haloalkyl, or alkoxy.

Embodiment 32. The method of any one of Embodiments 1-4, wherein in the compound of Formula (A), (I), or (II), or a salt thereof, R¹ is

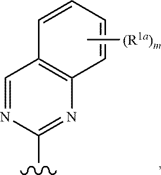

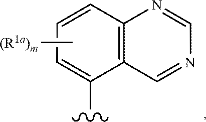

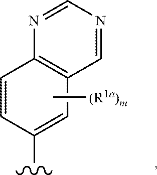

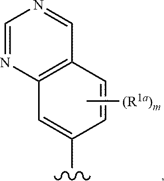

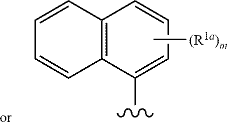

wherein m is 0, 1, 2, 3, 4, or 5 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 33. The method of any one of Embodiments 1-4, wherein in the compound of Formula (A), (I), or (II), or a salt thereof, R¹ is

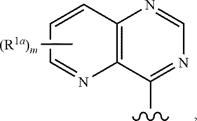

wherein m is 0, 1, 2, 3, or 4, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 34. The method of Embodiment 33, wherein in the compound of Formula (A), (I), or (II), or a salt thereof, R¹ is selected from the group consisting of

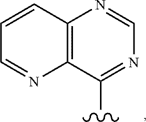

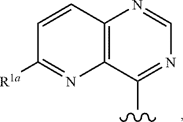

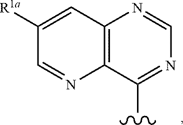

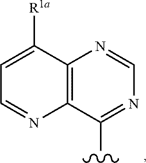

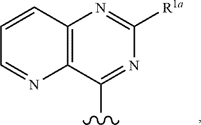

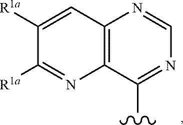

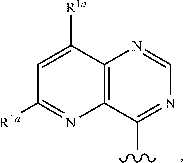

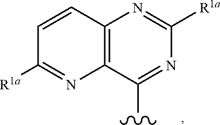

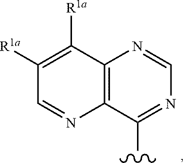

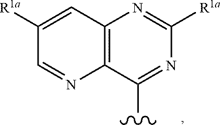

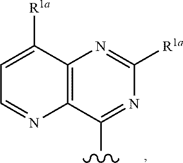

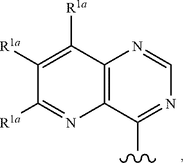

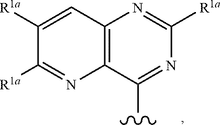

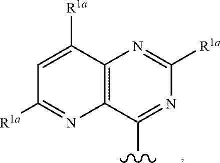

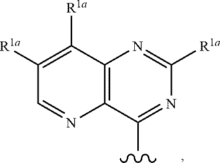

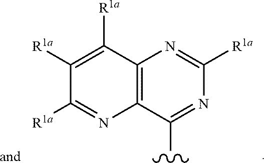

Embodiment 35. The method of any one of Embodiments 1-4, wherein in the compound of Formula (A), (I), or (II), or a salt thereof, R¹ is

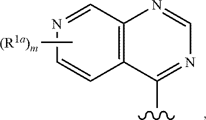

wherein m is 0, 1, 2, 3, or 4, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 36. The method of Embodiment 35, wherein in the compound of Formula (A), (I), or (II), or a salt thereof, R¹ is selected from the group consisting of

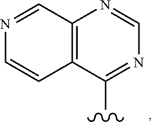

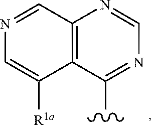

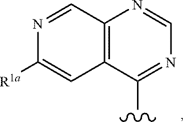

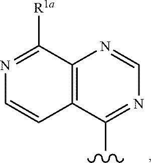

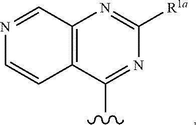

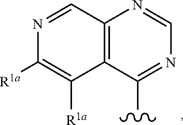

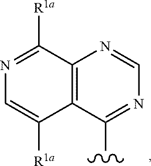

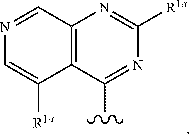

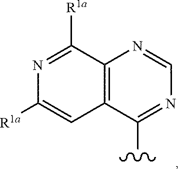

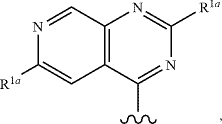

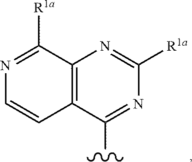

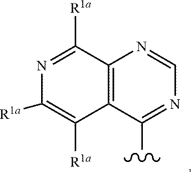

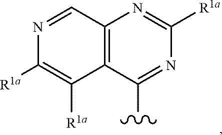

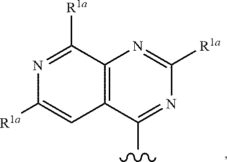

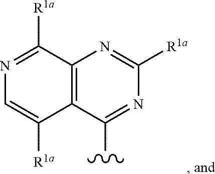

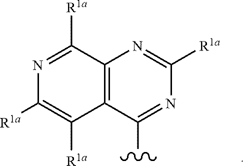

Embodiment 37. The method of any one of Embodiments 1-4, wherein in the compound of Formula (A), (I), or (II), or a salt thereof, R¹ is

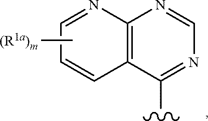

wherein m is 0, 1, 2, 3, or 4, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 38. The method of Embodiment 37, wherein in the compound of Formula (A), (I), or (II), or a salt thereof, R¹ is selected from the group consisting of

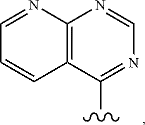

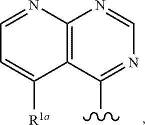

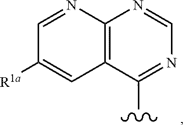

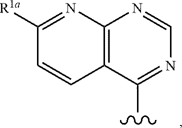

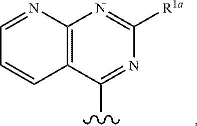

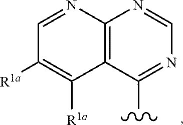

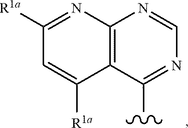

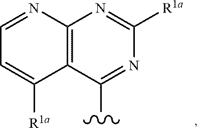

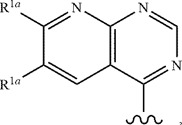

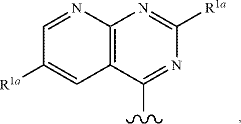

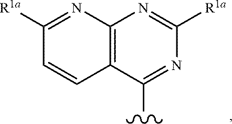

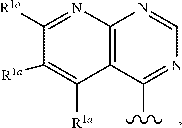

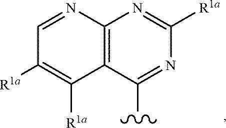

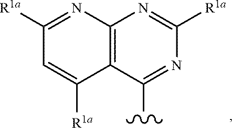

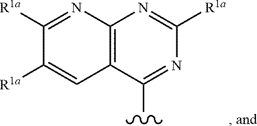

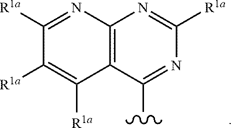

Embodiment 39. The method of any one of Embodiments 1-4, wherein in the compound of Formula (A), (I), or (II), or a salt thereof, R¹ is

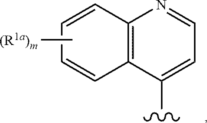

wherein m is 0, 1, 2, 3, 4, 5, or 6 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 40. The method of any one of Embodiments 1-4, wherein in the compound of Formula (A), (I), or (II), or a salt thereof, R¹ is

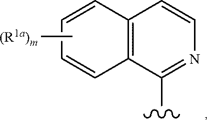

wherein m is 0, 1, 2, 3, 4, 5, or 6 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 41. The method of any one of Embodiments 1-4, wherein in the compound of Formula (A), (I), or (II), or a salt thereof, R¹ is

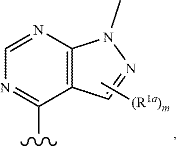

wherein m is 0, 1, or 2 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium.

Embodiment 42. The method of any one of Embodiments 1-4, wherein in the compound of Formula (A), (I), or (II), or a salt thereof, R¹ is selected from the group consisting of

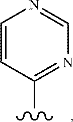

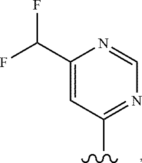

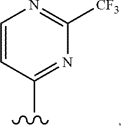

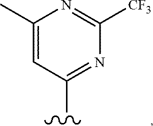

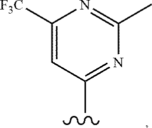

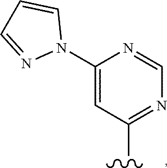

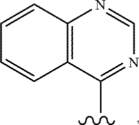

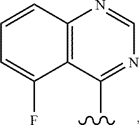

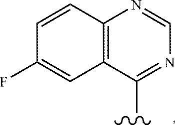

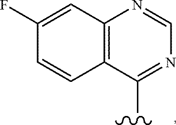

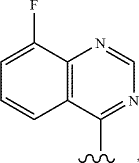

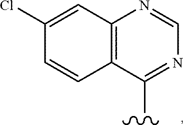

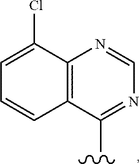

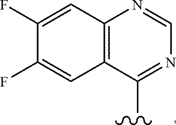

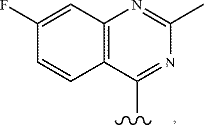

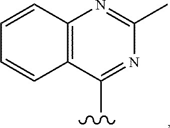

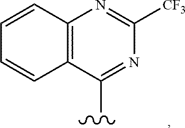

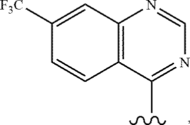

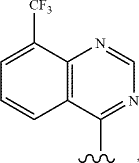

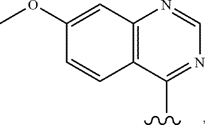

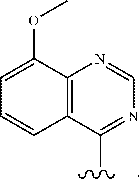

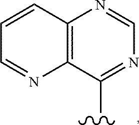

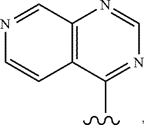

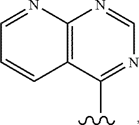

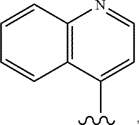

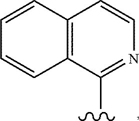

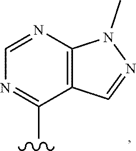

and any of the foregoing groups wherein any one or more hydrogen atom(s) are replaced with deuterium atom(s).

Embodiment 43. The method of any one of Embodiments 1-4, wherein in the compound of Formula (A), (I), or (II), or a salt thereof, R¹ is selected from the group consisting of

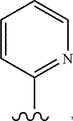

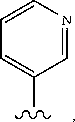

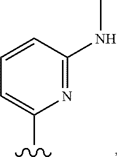

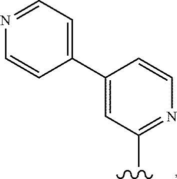

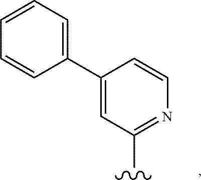

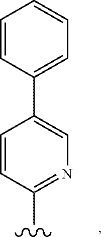

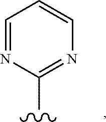

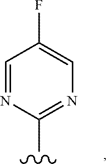

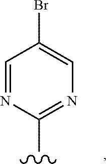

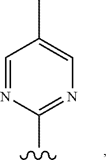

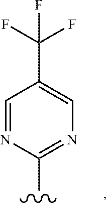

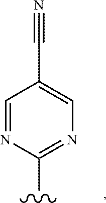

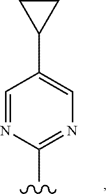

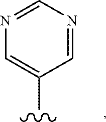

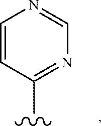

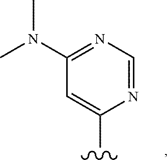

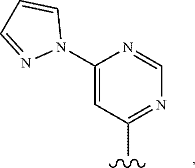

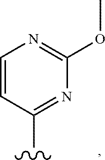

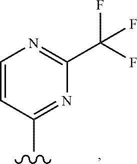

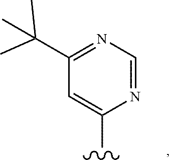

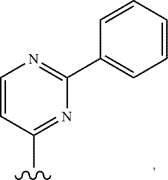

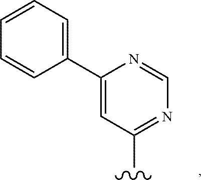

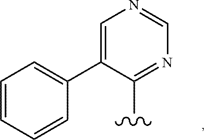

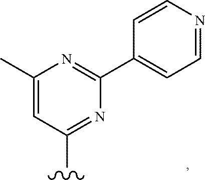

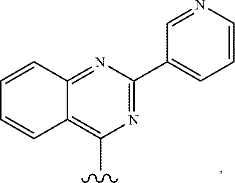

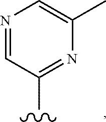

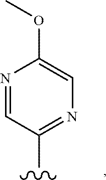

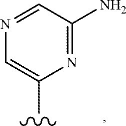

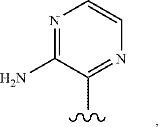

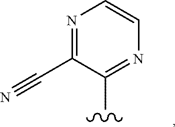

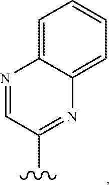

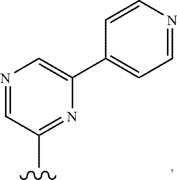

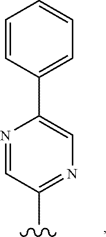

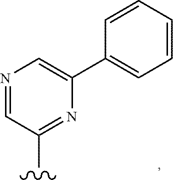

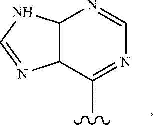

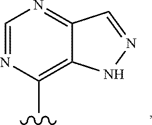

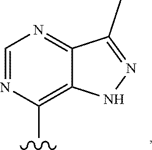

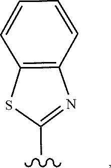

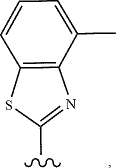

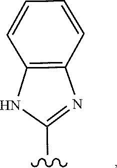

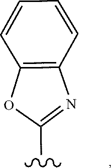

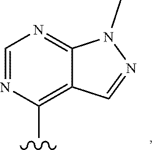

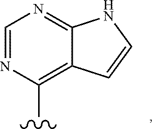

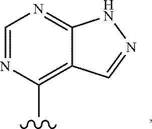

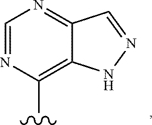

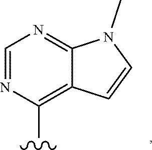

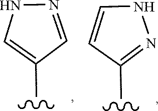

and any of the foregoing groups wherein any one or more hydrogen atom(s) are replaced with deuterium atom(s).

Embodiment 44. The method of any one of Embodiments 1-4, wherein in the compound of Formula (A), (I), or (II), or a salt thereof, R¹ is selected from the group consisting of

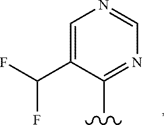

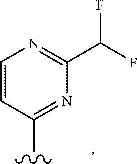

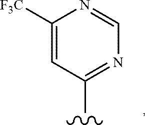

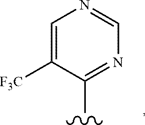

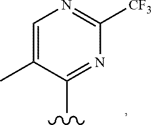

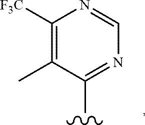

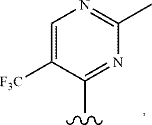

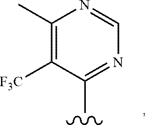

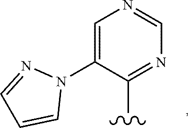

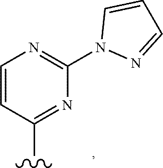

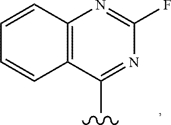

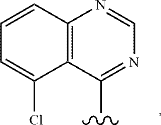

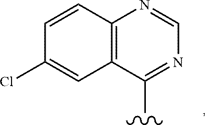

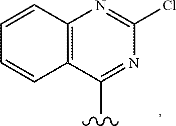

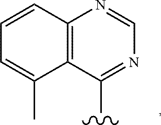

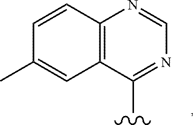

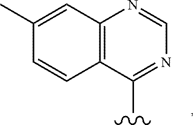

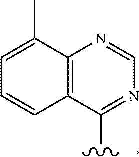

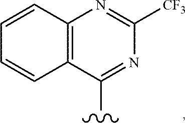

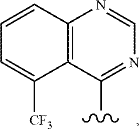

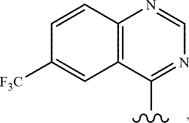

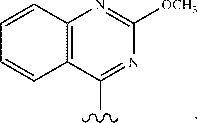

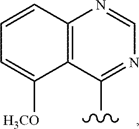

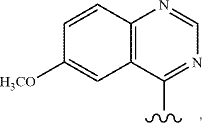

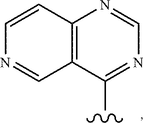

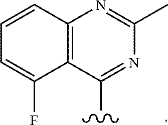

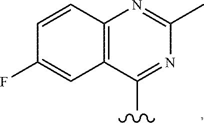

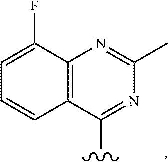

and any of the foregoing groups wherein any one or more hydrogen atom(s) are replaced with deuterium atom(s).

Embodiment 45. The method of any one of Embodiments 1-4, wherein in the compound of Formula (A), (I), or (II), or a salt thereof, R¹ is selected from the group consisting of

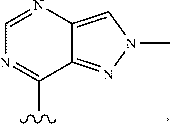

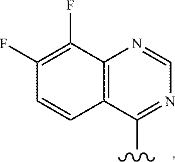

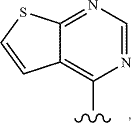

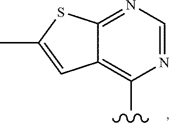

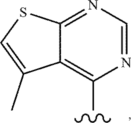

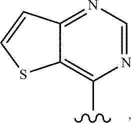

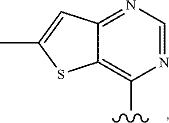

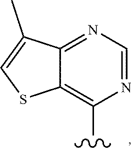

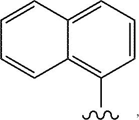

and any of the foregoing groups wherein any one or more hydrogen atom(s) are replaced with deuterium atom(s).

Embodiment 46. The method of any one of Embodiments 1 to 14 or 27-45, wherein in the compound of Formula (A), (I), or (II), or a salt thereof, R² is

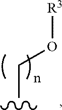

wherein nis 1, 2, 3, 4, 5, or 6, and R³ is C₁-C₂ alkyl optionally substituted by fluoro; phenyl optionally substituted by fluoro; pyridinyl optionally substituted by fluoro or methyl; or cyclopropyl optionally substituted by fluoro.

Embodiment 47. The method of any one of Embodiments 1 to 14 or 27-45, wherein in the compound of Formula (A), (I), or (II), or a salt thereof, R² is selected from the group consisting of

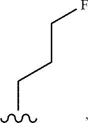

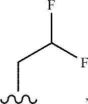

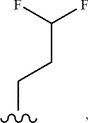

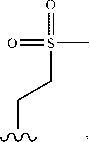

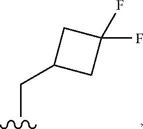

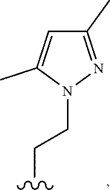

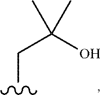

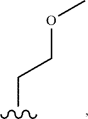

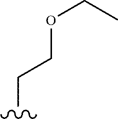

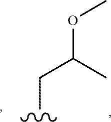

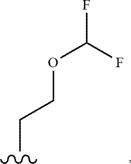

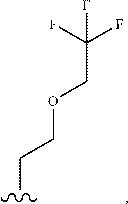

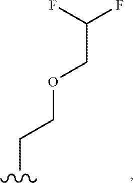

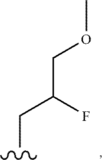

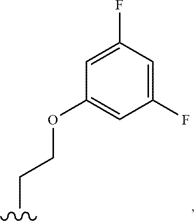

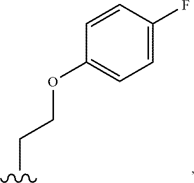

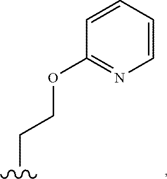

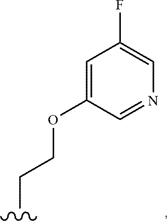

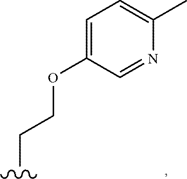

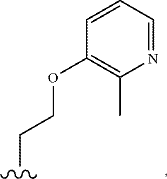

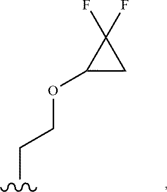

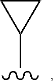

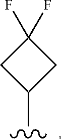

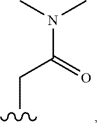

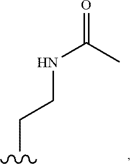

and any of the foregoing groups wherein any one or more hydrogen atom(s) are replaced with deuterium atom(s).

Embodiment 48. The method of any one of Embodiments 1 to 14 or 27-45, wherein in the compound of Formula (A), (I), or (II), or a salt thereof, R² is selected from the group consisting of

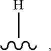

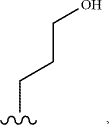

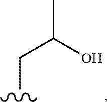

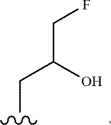

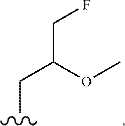

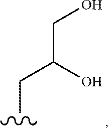

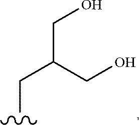

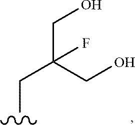

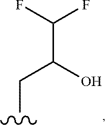

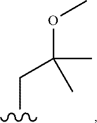

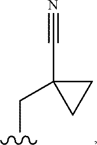

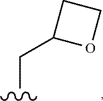

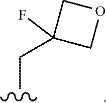

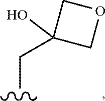

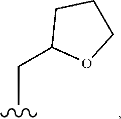

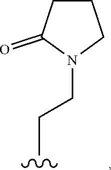

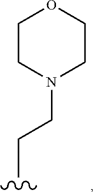

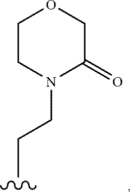

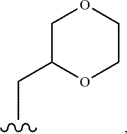

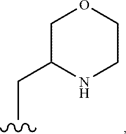

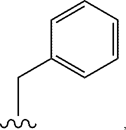

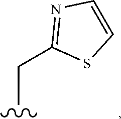

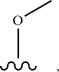

and any of the foregoing groups wherein any one or more hydrogen atom(s) are replaced with deuterium atom(s).

Embodiment 49. The method of any one of Embodiments 1 to 11, wherein in the compound of Formula (A), (I), or (II), or a salt thereof, R² is C₃-C₅ alkyl substituted by both fluorine and -OCH₃.

Embodiment 50. The method of any one of Embodiments 1 to 14 or 27-45, wherein in the compound of Formula (A), (I), or (II), or a salt thereof, R² is C₁-C₆ alkyl optionally substituted by —OR³, and R³ is phenyl optionally substituted by fluorine.

Embodiment 51. The method of any one of Embodiments 1 to 14 or 27-45, wherein in the compound of Formula (A), (I), or (II), or a salt thereof, R² is C₁-C₆ alkyl optionally substituted by —OR³, and R³ is pyridinyl optionally substituted by fluorine or methyl.

Embodiment 52. The method of any one of Embodiments 1 to 14 or 27-45, wherein in the compound of Formula (A), (I), or (II), or a salt thereof, R² is C₁-C₆ alkyl substituted by R^{2a} wherein R^{2a} is halogen.

Embodiment 53. The method of any one of Embodiments 1 to 14 or 27-45, wherein in the compound of Formula (A), (I), or (II), or a salt thereof, R² is C₁-C₆ alkyl substituted by R^{2a} wherein R^{2a} is deuterium.

Embodiment 54. The method of any one of Embodiments 1 to 14 or 27-45, wherein in the compound of Formula (A), (I), or (II), or a salt thereof, R² is C₁-C₆ alkyl substituted by R^{2a} wherein R^{2a} is 3- to 12-membered heterocyclyl optionally substituted by oxo.

Embodiment 55. The method of any one of Embodiments 1 to 14 or 27-45, wherein in the compound of Formula (A), (I), or (II), or a salt thereof, R² is C₁-C₆ alkyl substituted by R^{2a} wherein R^{2a} is 4- to 5-membered heterocyclyl optionally substituted by oxo.

Embodiment 56. The method of any one of Embodiments 1 to 14 or 27-45, wherein in the compound of Formula (A), (I), or (II), or a salt thereof, R² is C₁-C₆ alkyl substituted by R^{2a} wherein R^{2a} is C₆-C₁₄ aryl optionally substituted by halogen or -OR⁶.

Embodiment 57. The method of any one of Embodiments 1 to 14 or 27-45, wherein in the compound of Formula (A), (I), or (II), or a salt thereof, R² is C₁-C₆ alkyl substituted by R^{2a} wherein R^{2a} is phenyl optionally substituted by halogen or -OR⁶.

Embodiment 58. The method of any one of Embodiments 1 to 14 or 27-45, wherein in the compound of Formula (A), (I), or (II), or a salt thereof, R² is C₁-C₆ alkyl substituted by R^{2a} wherein R^{2a} is 5- to 10-membered heteroaryl optionally substituted by C₁-C₆ alkyl.

Embodiment 59. The method of any one of Embodiments 1 to 14 or 27-45, wherein in the compound of Formula (A), (I), or (II), or a salt thereof, R² is C₁-C₆ alkyl substituted by R^{2a} wherein R^{2a} is pyrazolyl optionally substituted by methyl.

Embodiment 60. The method of any one of Embodiments 1 to 14 or 27-45, wherein in the compound of Formula (A), (I), or (II), or a salt thereof, R² is C₁-C₆ alkyl substituted by R^{2a} wherein R^{2a} is C₃-C₈ cycloalkyl optionally substituted by —CN, halogen, or -OR⁶.

Embodiment 61. The method of any one of Embodiments 1 to 14 or 27-45, wherein in the compound of Formula (A), (I), or (II), or a salt thereof, R² is C₁-C₆ alkyl substituted by R^{2a} wherein R^{2a} is -S(O)₂R³.

Embodiment 62. The method of any one of Embodiments 1-4, wherein in the compound of Formula (A), (I), or (II), or a salt thereof, R¹ is pyridyl optionally substituted by R^{1a}.

Embodiment 63. The method of any one of Embodiments 1-4, wherein in the compound of Formula (A), (I), or (II), or a salt thereof, R¹ is indazolyl optionally substituted by R^{1a}.

Embodiment 64. The method of any one of Embodiments 1-4, wherein in the compound of Formula (A), (I), or (II), or a salt thereof, R¹ is 1H-pyrrolopyridyl optionally substituted by R^{1a}.

Embodiment 65. The method of any one of Embodiments 1-4, wherein in the compound of Formula (A), (I), or (II), or a salt thereof, R¹ is quinolinyl optionally substituted by R^{1a}.

Embodiment 66. The method of any one of Embodiments 1-4, wherein in the compound of Formula (A), (I), or (II), or a salt thereof, R¹ is phenyl optionally substituted by R^{1a}.

Embodiment 67. The method of any one of Embodiments 1-4, wherein in the compound of Formula (A), (I), or (II), or a salt thereof, R¹ is indanyl optionally substituted by R^{1a}.

Embodiment 68. The method of any one of Embodiments 1-67, wherein the compound of Formula (A), (I), or (II), or a salt thereof, is selected from Compound Nos. 1-66 in FIG. 1.

Embodiment 69. The method of any one of Embodiments 1-67, wherein the compound of Formula (A), (I), or (II), or a salt thereof, is selected from Compound Nos. 1-147.

Embodiment 70. The method of any one of Embodiments 1-67, wherein the compound of Formula (A), (I), or (II), or a salt thereof, is selected from Compound Nos. 1-665.

Embodiment 71. The method of any one of Embodiments 1-67, wherein the compound of Formula (A), (I), or (II), or a salt thereof, is selected from Compound Nos. 1-780.

Embodiment 72. The method of any one of Embodiments 1-67, wherein the compound of Formula (A), (I), or (II), or a salt thereof, is (S)-4-((2-methoxyethyl)(4-(5,6,7,8-tetrahydro-1,8-naphthyridin-2-yl)butyl)amino)-2-(quinazolin-4-ylamino)butanoic acid:

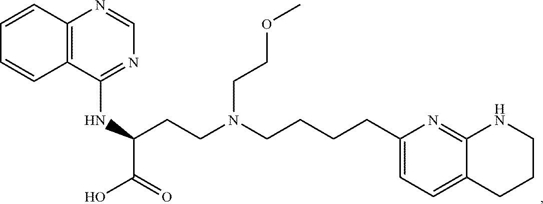

or a salt thereof.

Embodiment 73. The method of any one of Embodiments 1-72, comprising administering the compound of Formula (A), (I), or (II), or a salt thereof, in an amount in milligrams of about 1, 2.5, 5, 7.5, 10, 15, 20, 25, 30, 35, 40, 50, 75, 80, 85, 90, 95, 100, 105, 110, 115, 120, 125, 150, 160, 175, 200, 225, 250, 320, 400, 480, 560, 640, 720, 800, 880, 960, or 1040, or a range between any two of the preceding values.

Embodiment 74. The method of any one of Embodiments 1-72, comprising administering the compound of Formula (A), (I), or (II), or a salt thereof, in an amount effective on administration to the subject to produce a Cₘₐₓ in plasma of the subject in ng/mL of at least about one of 700, 750, 800, 850, 900, 950, 1000, 1050, 1100, 1150, 1200, 1250, 1300, 1350, 1400, 1450, 1500, 1600, 1700, 1800, 1900, 2000, 2100, 2200, 2300, 2400, or 2500, or a range between any two of the preceding concentrations.

Embodiment 75. The method of any one of Embodiments 1-72, comprising administering the compound of Formula (A), (I), or (II), or a salt thereof, in an amount effective on administration to the subject to produce a Cₘₐₓ in plasma of the subject in ng/mL, the Cₘₐₓ corresponding to a plasma-adjusted concentration effective to inhibit a percentage of αvβ₆ or αvβ₁ in the individual of at least about one of 50, 55, 60, 65, 70, 75, 80, 85, 90, 95, or 100, or a range between any two of the preceding percentages.

Embodiment 76. The method of any one of Embodiments 1-75, comprising administering the compound of Formula (A), (I), or (II), or a salt thereof, daily to the subject.

Embodiment 77. The method of any one of Embodiments 1-75, comprising administering the compound of Formula (A), (I), or (II), or a salt thereof, once daily to the subject.

Embodiment 78. The method of any one of Embodiments 1-75, wherein the daily administering is given one time, two times, three times, or four times daily.

Embodiment 79. The method of any one of Embodiments 76-78, wherein the daily administering is given once daily.

Embodiment 80. The method of any one of Embodiments 1-79, wherein the disease is a pulmonary disease.

Embodiment 81. The method of any one of Embodiments 1-79, wherein the disease is a fibrotic disease.

Embodiment 82. The method of any one of Embodiments 1-79, wherein the disease is a pulmonary fibrotic disease.

Embodiment 83. The method of any one of Embodiments 1-79, wherein the disease is selected from the group consisting of: idiopathic pulmonary fibrosis, an interstitial lung disease, radiation-induced pulmonary fibrosis, systemic scleroderma or systemic sclerosis associated interstitial lung disease, and nonspecific interstitial pneumonia.

Embodiment 84. The method of any one of Embodiments 1-83, wherein the second drug is pirfenidone, represented by:

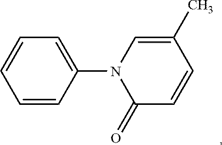

or a salt thereof; or the systematic chemical name 5-methyl-1phenyl-2-1(H)-pyridone, or a salt thereof.

Embodiment 85. The method of Embodiment 84, wherein the pirfenidone or a salt thereof is orally administered.

Embodiment 86. The method of Embodiment 85, wherein the pirfenidone or a salt thereof is orally administered to the subject via at least one of a capsule dosage form and a tablet dosage form.

Embodiment 87. The method of Embodiment 86, wherein the pirfenidone or a salt thereof is orally administered to the subject via the capsule dosage form.

Embodiment 88. The method of Embodiment 87, wherein the capsule dosage form comprises the pirfenidone or a salt thereof and 1, 2, 3, or 4 ingredients selected from the group consisting of: microcrystalline cellulose, croscarmellose sodium, povidone, and magnesium stearate.

Embodiment 89. The method of Embodiment 87, wherein at least one of:
the capsule dosage form is characterized by an amount per capsule of the pirfenidone of one of, or about one of: 267 mg, 534 mg, or 801 mg, or a range between any two of the preceding values; or
the amount of pirfenidone orally administered to the subject via the capsule dosage form in a single administration event is one of, or about one of: 267 mg, 534 mg, or 801 mg, or a range between any two of the preceding values.

Embodiment 90. The method of Embodiment 87, wherein a capsule shell of the capsule dosage form comprises gelatin and titanium dioxide.

Embodiment 91. The method of Embodiment 86, wherein the pirfenidone or a salt thereof pirfenidone or a salt thereof is orally administered to the subject via the tablet dosage form.

Embodiment 92. The method of Embodiment 91, wherein the tablet dosage form comprises the pirfenidone or a salt thereof and 1, 2, 3, 4, 5, 6, 7, 8, 9, or 10 ingredients selected from the group consisting of: Microcrystalline cellulose, colloidal anhydrous silica, povidone, croscarmellose sodium, magnesium stearate, polyvinyl alcohol, titanium dioxide, macrogol (polyethylene glycol), talc, and iron oxide.

Embodiment 93. The method of Embodiment 91, wherein at least one of:
the tablet dosage form is characterized by an amount per capsule of the pirfenidone of one of, or about one of: 267 mg, 534 mg, or 801 mg, or a range between any two of the preceding values; or
the amount of pirfenidone orally administered to the subject via the tablet dosage form in a single administration event is one of, or about one of: 267 mg, 534 mg, or 801 mg, or a range between any two of the preceding values.

Embodiment 94. The method of Embodiment 91, wherein the tablet dosage form comprises an outer coating.

Embodiment 95. The method of Embodiment 85, wherein upon initiation of treatment with the pirfenidone or a salt thereof, the pirfenidone is titrated to a full daily dosage over a period of time.

Embodiment 96. The method of Embodiment 95, wherein upon initiation of treatment with the pirfenidone or a salt thereof, the pirfenidone is titrated to a full daily dosage over a 14-day period as follows:
days 1 through 7, 267 mg three times daily to achieve a daily pirfenidone dosage of 801 mg/day;
days 8 through 14, 534 mg three times daily to achieve a daily pirfenidone dosage of 1602 mg/day; and
days 15 onward, 801 mg three times daily to achieve the full daily pirfenidone dosage of 2403 mg/day.

Embodiment 97. The method of Embodiment 85, wherein the pirfenidone or a salt thereof is administered in a full daily pirfenidone dosage of 2403 mg/day.

Embodiment 98. The method of Embodiment 85, wherein the disease is idiopathic pulmonary fibrosis.

Embodiment 99. The method of Embodiment 85, wherein the pirfenidone is administered as a granulate formulation of 5-methyl-1-phenyl-2-(1H)-pyridone, characterized by one of:
5-methyl-1-phenyl-2-(1H)-pyridone and pharmaceutically acceptable excipients, said excipients comprising an effective amount of binder to increase the AUC of the 5-methyl-1-phenyl-2-(1H)-pyridone at least 45% upon oral administration, as compared to pirfenidone without excipients orally administered in a capsule shell; or
granules comprising 5-methyl-1-phenyl-2-(1H)-pyridone and a glidant, and one or more extragranular excipients comprising an extragranular glidant.

Embodiment 100. The method of Embodiment 85, wherein the pirfenidone is administered as a coated tablet dosage form comprising a compressed tablet comprising 5-methyl-1-phenyl-2-(1H)-pyridone as an active ingredient; and a coating comprising a light shielding agent disposed on the compressed tablet.

Embodiment 101. The method of Embodiment 85, wherein the pirfenidone is administered as a capsule dosage form, wherein the capsule dosage form is characterized by one of:
a capsule comprising a pharmaceutical formulation of 5-methyl-1-phenyl-2-(1H)-pyridone, wherein said pharmaceutical formulation comprises 5-30% by weight of pharmaceutically acceptable excipients and 70-95% by weight of 5-methyl-1-phenyl-2-(1H)-pyridone, wherein said excipients comprise an effective amount of binder to increase the AUC of pirfenidone upon oral administration, as compared to a capsule comprising no excipients;
a capsule comprising a pharmaceutical formulation of 5-methyl-1-phenyl-2-(1H)-pyridone, wherein said pharmaceutical formulation comprises 5-methyl-1-phenyl-2-(1H)-pyridone and pharmaceutically acceptable excipients, said excipients comprising an effective amount of binder to increase the AUC of pirfenidone upon oral administration, as compared to a capsule comprising no excipients;
a capsule comprising a pharmaceutical formulation of 5-methyl-1-phenyl-2-(1H)-pyridone, wherein said pharmaceutical formulation comprises pharmaceutically acceptable excipients and 5-methyl-1-phenyl-2-(1H)-pyridone, and the formulation is stable for at least 9 months at 40° C. at 75% relative humidity, as measured by a dissolution of at least 85% of the 5-methyl-1-phenyl-2-(1H)-pyridone after the at least 9 months; or
a capsule comprising a pharmaceutical formulation of 5 methyl-1-phenyl-2-(1H)-pyridone, wherein said pharmaceutical formulation comprises pharmaceutically acceptable excipients and 5-methyl-1-phenyl-2-(1H)-pyridone, and the formulation is stable for at least 18 months at 25° C. at 60% relative humidity, as measured by a dissolution of at least 93% of the 5-methyl-1-phenyl-2-(1H)-pyridone after the at least 18 months;

Embodiment 102. The method of any one of Embodiments 1-83, wherein the second drug is nintedanib or a salt thereof, and is represented by one or both of:

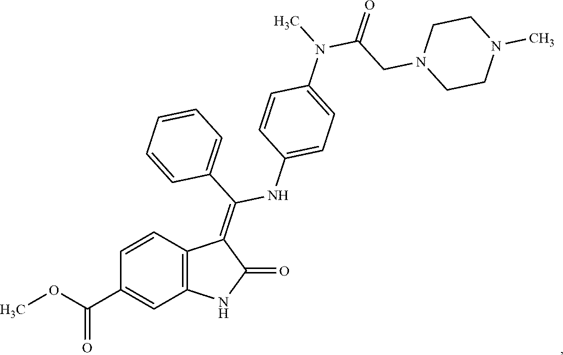

or a salt thereof; orthe systematic chemical name 3-Z-[1-(4-(N-((4-methyl-piperazin-1-yl)-methylcarbonyl)-N-methyl-amino)-anilino)-1-phenyl-methylene]-6-methoxycarbonyl-2-indolinone, or a salt thereof.

Embodiment 103. The method of Embodiment 102, wherein the salt of nintedanib is represented by one or both of:

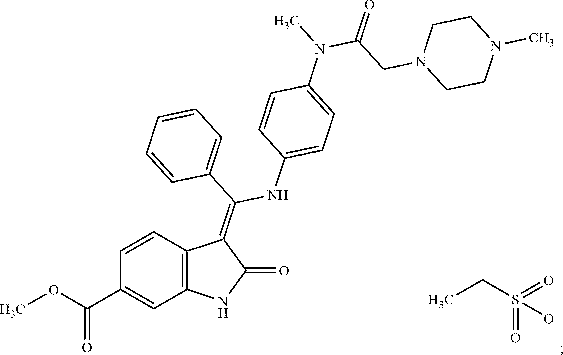

or the systematic chemical name 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate.

Embodiment 104. The method of Embodiments 102 or 103, wherein the the nintedanib or a salt thereof is characterized as one or more of:
3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate in crystalline form, having a melting point of Tm.p.=305±5° C. (determined by DSC; evaluation using peak-maximum; heating rate: 10° C./min);
crystalline 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate according to claim 2, the X-ray powder diagram of which includes, inter alia, the characteristic values d=5.43 Å, 5.08 Å, 4.71 Å, 4.50 Å and 4.43 Å with an intensity of more than 40%;
crystalline 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate according to claim 2, characterised by a unit cell determined by X-ray powder diffractometric measurements having the following dimensions: a=16.332 Å, b=19.199 Å, c=11.503 Å, α=95.27°, β=90.13°, γ=110.83° and V=3354.4 Å3;
a pharmaceutical composition comprising 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate and one or more inert carriers and/or diluents;
a prodrug of 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate; or 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate in crystalline form.

Embodiment 105. The method of any of Embodiments 102-104, wherein the nintedanib or salt thereof is orally administered.

Embodiment 106. The method of any one of Embodiments 102-105, wherein the nintedanib or a salt thereof is orally administered to the subject via at least one of a lipid dosage form and a capsule dosage form.

Embodiment 107. The method of Embodiment 106, wherein at least one of:
the lipid dosage form is characterized by an amount of the nintedanib or salt thereof equivalent to, or equivalent to about, 100 mg or 150 mg of nintedanib, or a range between about 100 mg to about 150 mg of nintedanib; or
the amount of nintedanib or salt thereof orally administered to the subject via the lipid dosage form in a single administration event is equivalent to, or equivalent to about, 100 mg or 150 mg of nintedanib, or a range between about 100 mg to about 150 mg of nintedanib.

Embodiment 108. The method of Embodiment 106, wherein at least one of:
the lipid dosage form is characterized by an amount of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib; or
the amount of nintedanib or salt thereof orally administered to the subject via the lipid dosage form in a single administration event is characterized by an amount per capsule of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib.

Embodiment 109. The method of Embodiment 106, wherein the nintedanib or a salt thereof is orally administered to the subject via the lipid dosage form, the lipid dosage form characterized by one or more of:
(a) a formulation of the active substance 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate which comprises a lipid suspension of the active substance in 1 to 90 wt. % of medium chain triglycerides, 1 to 30 wt. % of hard fat and 0.1 to 10 wt. % of lecithin;
(b) a pharmaceutical dosage form which is a viscous lipid suspension formulation comprising:
   10 to 50 wt. % of the active substance 3-Z-[1-(4-(N-((4-methyl-piperazin-1-yl)-methylcarbonyl)-N-methyl-amino)-anilino)-1-phenyl-methylenel]-6-methoxycarbonyl-2-indolinone-monoethanesulphonate,
   10 to 70 wt. % of medium chain triglycerides;
   10 to 30 wt. % of hard fat; and
   0.25 to 2.5 wt. % of lecithin,
   which delivers an immediate release profile in which not less than 70% (Q65%) of the active substance is dissolved in 60 minutes in vitro under the following in vitro dissolution conditions according to European Pharmacopeia 6.2: Apparatus 2 (paddle), dissolution medium with 0.1 M HCl (pH 1) and stirring speed of 50 to 150 rpm, at a temperature of 37° C.; or
(c) a lipid suspension comprising, consisting of, or consisting essentially of 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate, medium chain triglycerides, hard fat and lecithin, wherein the medium chain triglycerides, hard fat and lecithin are present in the lipid suspension in the following amounts:
   1 to 90 wt. % of medium chain triglycerides,
   1 to 30 wt. % of hard fat, and
   0.1 to 10 wt. % of lecithin.

Embodiment 110. The method of Embodiment 106, wherein the nintedanib or a salt thereof is orally administered to the subject via the capsule dosage form, the capsule dosage form comprising a capsule shell and a capsule formulation.

Embodiment 111. The method of Embodiment 106, wherein the nintedanib or a salt thereof is orally administered to the subject via the capsule dosage form, the capsule dosage form comprising a capsule shell and a capsule formulation, the capsule formulation comprising the lipid dosage form characterized by one or more of:
(a) a formulation of the active substance 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate which comprises a lipid suspension of the active substance in 1 to 90 wt. % of medium chain triglycerides, 1 to 30 wt. % of hard fat and 0.1 to 10 wt. % of lecithin;
(b) a pharmaceutical dosage form which is a viscous lipid suspension formulation comprising:
   10 to 50 wt. % of the active substance 3-Z-[1-(4-(N-((4-methyl-piperazin-1-yl)-methylcarbonyl)-N-methyl-amino)-anilino)-1-phenyl-methylenel]-6-methoxycarbonyl-2-indolinone-monoethanesulphonate,
   10 to 70 wt. % of medium chain triglycerides;
   10 to 30 wt. % of hard fat; and
   0.25 to 2.5 wt. % of lecithin,
   which delivers an immediate release profile in which not less than 70% (Q65%) of the active substance is dissolved in 60 minutes in vitro under the following in vitro dissolution conditions according to European Pharmacopeia 6.2: Apparatus 2 (paddle), dissolution medium with 0.1 M HCl (pH 1) and stirring speed of 50 to 150 rpm, at a temperature of 37° C.; or
(c) a lipid suspension comprising, consisting of, or consisting essentially of 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate, medium chain triglycerides, hard fat and lecithin, wherein the medium chain triglycerides, hard fat and lecithin are present in the lipid suspension in the following amounts:
   1 to 90 wt. % of medium chain triglycerides,
   1 to 30 wt. % of hard fat, and
   0.1 to 10 wt. % of lecithin.

Embodiment 112. The method of Embodiment 110, wherein at least one of:
the capsule dosage form is characterized by an amount per capsule of the nintedanib or salt thereof equivalent to, or equivalent to about, 100 mg or 150 mg of nintedanib, or a range between about 100 mg to about 150 mg of nintedanib; or
the amount of nintedanib or salt thereof orally administered to the subject via the capsule dosage form in a single administration event is equivalent to, or equivalent to about, 100 mg or 150 mg of nintedanib, or a range between about 100 mg to about 150 mg of nintedanib.

Embodiment 113. The method of Embodiment 110, wherein at least one of:
the capsule dosage form is characterized by an amount per capsule of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib; or
the amount of nintedanib or salt thereof orally administered to the subject via the capsule dosage form in a single administration event is characterized by an amount per capsule of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib.

Embodiment 114. The method of Embodiment 110, wherein the capsule shell of the capsule dosage form comprises 1, 2, 3, 4, 5, or 6 of: gelatin, glycerol, titanium dioxide, red ferric oxide, yellow ferric oxide, and black ink.

Embodiment 115. The method of any one of Embodiments 1-83 or 102-114, wherein the second drug is nintedanib or a salt thereof and is administered to the subject twice daily in a dosage of the nintedanib or a salt thereof equivalent to, or equivalent to about, 100 mg of nintedanib, for a total daily dose equivalent to, or equivalent to about, 200 mg of nintedanib.

Embodiment 116. The method of Embodiment 115, wherein the subject has one of a mild hepatic impairment or a side effect associated with nintedanib or a salt thereof.

Embodiment 117. The method of any one of Embodiments 1-83 or 102-114, wherein the second drug is nintedanib or a salt thereof and is administered to the subject twice daily in a dosage of the nintedanib or a salt thereof equivalent to, or equivalent to about, 150 mg of nintedanib, for a total daily dose equivalent to, or equivalent to about, 300 mg of nintedanib.

Embodiment 118. The method of any of Embodiments 102-117, wherein the disease is selected from the group consisting of idiopathic pulmonary fibrosis, an interstitial lung disease, and systemic sclerosis-associated interstitial lung disease.

Embodiment 119. The method of Embodiment 118, wherein the interstitial lung disease includes chronic fibrosing interstitial lung diseases (ILDs) with a progressive phenotype.

Embodiment 120. The method of Embodiment 118, wherein the disease includes systemic sclerosis-associated interstitial lung disease, and treating the subject includes slowing the rate of decline in pulmonary function in the subject associated with the systemic sclerosis-associated interstitial lung disease.

Embodiment 121. The method of any one of Embodiments 1-120, comprising administering the first drug to the subject in an amount effective to modulate at least one integrin in the subject.

Embodiment 122. The method of any one of Embodiments 1-120, wherein the subject has at least one tissue in need of therapy and the tissue has at least one elevated level of:
at least one integrin activity and/or expression;
a pSMAD/SMAD value;
new collagen formation or accumulation;
total collagen; and
Type I Collagen gene Col1a1 expression;
and wherein the level is elevated compared to a healthy state of the tissue.

Embodiment 123. The method of Embodiment 121 or 122, comprising administering the first drug to the subject in an amount effective to inhibit the at least one integrin in the subject.

Embodiment 124. The method of Embodiment 121 or 122, wherein the at least one integrin in the subject comprises αv.

Embodiment 125. The method of Embodiment 121 or 122, wherein the at least one integrin in the subject is selected from the group consisting of αvβ₆ integrin and αvβ₁ integrin.

Embodiment 126. The method of Embodiment 121 or 122, wherein the at least one integrin in the subject comprises both αvβ₆ integrin and αvβ₁ integrin.

Embodiment 127. The method of Embodiment 121 or 122, comprising administering the first drug to the subject in an amount effective to inhibit one or both of αvβ₁ integrin and αvβ₆ integrin in the subject.

Embodiment 128. The method of any of Embodiments 121-127, wherein the method selectively reduces αvβ₁ integrin activity and/or expression compared to αvβ₆ integrin activity and/or expression in the subject.

Embodiment 129. The method of any of Embodiments 121-127, wherein the method selectively reduces αvβ₆ integrin activity and/or expression compared to αvβ₁ integrin activity and/or expression in the subject.

Embodiment 130. The method of any of Embodiments 121-127, wherein the method reduces both αvβ₁ integrin and αvβ₆ integrin activity and/or expression compared to at least one other αv-containing integrin in the subject.

Embodiment 131. The method of any of Embodiments 121-127, wherein the activity of αvβ₁ integrin in one or more fibroblasts is reduced in the subject.

Embodiment 132. The method of any of Embodiments 121-127, wherein the activity of αvβ₆ integrin in one or more epithelial cells is reduced in the subject.

Embodiment 133. The method of Embodiment 122, wherein the at least one tissue in the subject comprises one or more of: lung tissue, liver tissue, skin tissue, cardiac tissue, kidney tissue, gastrointestinal tissue, gall bladder tissue, and bile duct tissue.

Embodiment 134. The method of any one of Embodiments 122-133, wherein the tissue has an elevated pSMAD2/SMAD2 value or an elevated pSMAD3/SMAD3 value compared to the healthy state of the tissue.

Embodiment 135. The method of any one of Embodiments 1-134, wherein the first drug and/or the second drug are administered orally to the subject.

Embodiment 136. The method of any one of Embodiments 1-135, wherein the first drug and/or the second drug are administered to the subject with food.

Embodiment 137. The method of any one of Embodiments 1-136, wherein the first drug and the second drug are administered to the subject at the same time or on a same schedule.

Embodiment 138. The method of any one of Embodiments 1-136, wherein the first drug and the second drug are administered to the subject at different times or on a different schedule.

Embodiment 139. The method of any one of Embodiments 1-136, wherein the second drug is administered to the subject over a period of days, weeks, months, or years before first administering the first drug to the subject.

Embodiment 140. The method of any one of Embodiments 1-139, wherein after administering the first and second drugs to the subject over a period of days, weeks, months, or years, the dose of the second drug is decreased in amount or frequency.

Embodiment 141. The method of any one of Embodiments 1-139, wherein after administering the first and second drugs to the subject over a period of days, weeks, months, or years, administration of the second drug is discontinued.

Embodiment 142. The method of Embodiment 140 or 141, wherein the second drug is decreased in amount or frequency or discontinued after the subject experiences a stabilization, improvement, or remission in the disease.

Embodiment 143. The method of any of Embodiments 1-142, wherein the subject is human.

Embodiment 144. A method of reducing decline in forced vital capacity (FVC) in a subject in need thereof, comprising administering (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof to the subject.

Embodiment 145. The method of embodiment 144, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in a therapeutically effective amount sufficient to reduce the decline in FVC in the subject as compared to a subject who has not been administered (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof.

Embodiment 146. The method of embodiment 144 or embodiment 145, wherein the administering is for at least about 12 weeks.

Embodiment 147. The method of embodiment 144 or embodiment 145, wherein the administering is for about a 12 week period.

Embodiment 148. The method of any one of embodiments 144-147, wherein the administering is daily.

Embodiment 149. The method of any one of embodiments 144-148, wherein the administering is once daily.

Embodiment 150. The method of any one of embodiments 144-149, wherein the decline in FVC is about 50 mL or less.

Embodiment 151. The method of any one of embodiments 144-149, wherein the decline in FVC is about 30 mL or less.

Embodiment 152. The method of any one of embodiments 144-149, wherein the decline in FVC is about 15 mL or less.

Embodiment 153. The method of any one of embodiments 144-149, wherein the administering is for about a 12 week period and the decline in FVC is about 50 mL or less from the start of the period to the end of the period.

Embodiment 154. The method of any one of embodiments 144-149, wherein the decline in FVC is about 30 mL or less from the start of the period to the end of the period.

Embodiment 155. The method of any one of embodiments 144-149, wherein the decline in FVC is about 15 mL or less from the start of the period to the end of the period.

Embodiment 156. The method of any of embodiments 144-155, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 40 mg daily, or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment 157. The method of any of embodiments 144-155, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 80 mg daily, or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment 158. The method of any of embodiments 144-155 wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 160 mg daily, or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment 159. The method of any of embodiments 144-155, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of at least about 700 ng/mL.

Embodiment 160. The method of any of embodiments 144-155, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,000 ng/mL plus or minus 200 ng/mL.

Embodiment 161. The method of any of embodiments 144-155, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,600 ng/mL plus or minus 300 ng/mL.

Embodiment 162. The method of any of embodiments 144-155, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 2,700 ng/mL plus or minus 400 ng/mL.

Embodiment 163. A method of increasing forced vital capacity (FVC) in a subject in need thereof, comprising administering (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof to the subject.

Embodiment 164. The method of embodiment 163, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in a therapeutically effective amount sufficient to increase FVC in the subject as compared to a subject who has not been administered (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof.

Embodiment 165. The method of embodiment 163 or embodiment 164, wherein the administering is for at least about 12 weeks.

Embodiment 166. The method of embodiment 163 or embodiment 164, wherein the administering is for about a 12 week period.

Embodiment 167. The method of any one of embodiments 163-166, wherein the administering is daily.

Embodiment 168. The method of any one of embodiments 163-166, wherein the administering is once daily.

Embodiment 169. The method of any one of embodiments 163-168, wherein the increase in FVC is about 10 mL or more.

Embodiment 170. The method of any one of embodiments 163-168, wherein the increase in FVC is about 20 mL or more.

Embodiment 171. The method of any one of embodiments 163-168, wherein the increase in FVC is about 30 mL or more.

Embodiment 172. The method of any one of embodiments 163-168, wherein the administering is for about a 12 week period and the increase in FVC is about 10 mL or more from the start of the period to the end of the period.

Embodiment 173. The method of any one of embodiments 163-168, wherein the increase in FVC is about 20 mL or more from the start of the period to the end of the period.

Embodiment 174. The method of any one of embodiments 163-168, wherein the increase in FVC is about 30 mL or more from the start of the period to the end of the period.

Embodiment 175. The method of any one of embodiments 163-174, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 40 mg daily, or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment 176. The method of any one of embodiments 163-174, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 80 mg daily, or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment 177. The method of any one of embodiments 163-174, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 160 mg daily, or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment 178. The method of any one of embodiments 163-174, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of at least about 700 ng/mL.

Embodiment 179. The method of any one of embodiments 163-174, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,000 ng/mL plus or minus 200 ng/mL.

Embodiment 180. The method of any one of embodiments 163-174, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,600 ng/mL plus or minus 300 ng/mL.

Embodiment 181. The method of any one of embodiments 163-174, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 2,700 ng/mL plus or minus 400 ng/mL.

Embodiment 182. The method of any one of embodiments 144-181, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in a therapeutically effective amount.

Embodiment 183. The method of any one of embodiments 144-182, wherein the pharmaceutically acceptable salt is a phosphate salt of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 184. The method of any one of embodiments 144-183, wherein the subject has a fibrotic lung disease.

Embodiment 185. The method of embodiment 184, wherein the fibrotic lung disease is idiopathic pulmonary fibrosis (IPF).

Embodiment 186. The method of any one of embodiments 144-185, wherein the subject is a human.

Embodiment 187. The method of any one of embodiments 144-186, wherein the subject is concurrently being treated with a standard medical therapy or a standard of care.

Embodiment 188. The method of embodiment 187, wherein the standard medical therapy or standard of care comprises administration of pirfenidone, administration of nintedanib, or administration of pirfenidone and nintedanib.

Embodiment 189. The method of any one of embodiments 144-188, wherein the subject is not being concurrently treated with a standard medical therapy or a standard of care.

Embodiment 190. The method of embodiment 189, wherein the standard medical therapy or standard of care comprises administration of pirfenidone, administration of nintedanib, or administration of pirfenidone and nintedanib.

Embodiment 191. The method of any one of embodiments 144-186, wherein the subject is not administered any treatment other than (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof.

Embodiment 192. The method of any one of embodiments 144-191, wherein the method is not accompanied by a serious adverse event.

Embodiment 193. The method of embodiment 192, wherein the serious adverse event is a gastrointestinal adverse event.

Embodiment 194. The method of any one of embodiments 144-191, wherein the incidence of adverse events is lower than the incidence of adverse events for a standard medical therapy or a standard of care.

Embodiment 195. The method of embodiment 194, wherein the standard medical therapy or standard of care comprises administration of pirfenidone, administration of nintedanib, or administration of pirfenidone and nintedanib.

Embodiment 196. The method of embodiment 194 or embodiment 195, wherein the adverse events are gastrointestinal adverse events.

Embodiment C-1. A method of treating a subject for a disease, comprising: administering to the subject a first drug comprising a compound of formula (II):

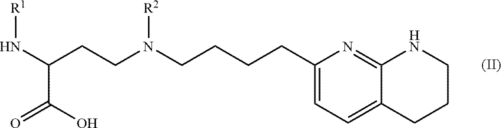

or a salt thereof; and
administering to the subject at least a second drug that is selected from the group consisting of: pirfenidone and nintedanib, or a salt thereof, whereby the subject is treated for the disease;
wherein in the compound of Formula (II):
   R¹ is C₆-C₁₄ aryl or 5- to 10-membered heteroaryl wherein the C₆-C₁₄ aryl and 5- to 10-membered heteroaryl are optionally substituted by R^{1a};
   R² is hydrogen; deuterium; C₁-C₆ alkyl optionally substituted by R^{2a}; —OH; —O—C₁—C₆ alkyl optionally substituted by R^{2a}; C₃-C₆ cycloalkyl optionally substituted by R^{2b}; —O—C₃—C₆ cycloalkyl optionally substituted by R^{2b}; 3- to 12-membered heterocyclyl optionally substituted by R^{2c}; or -S(O)₂R^{2d}; with the proviso that any carbon atom bonded directly to a nitrogen atom is optionally substituted with an R^{2a} moiety other than halogen; each R^{1a} is independently C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, C₆-C₁₄ aryl, deuterium, halogen, —CN, -OR³, -SR³, -NR⁴R⁵, —NO₂, -C=NH(OR³), -C(O)R³, —OC(O)R³,
   -C(O)OR³, -C(O)NR⁴R⁵, -NR³C(O)R⁴, -NR³C(O)OR⁴, -NR³C(O)NR⁴R⁵, -S(O)R³, -S(O)₂R³,
   -NR³S(O)R⁴, -NR³S(O)₂R⁴, -S(O)NR⁴R⁵, —S(O)₂NR4R5, or -P(O)(OR⁴)(OR⁵), wherein each R^{1a}is, where possible, independently optionally substituted by deuterium, halogen, oxo,
   -OR⁶, -NR⁶R⁷, -C(O)R⁶, —CN, -S(O)R⁶, -S(O)₂R⁶, -P(O)(OR⁶)(OR⁷), C₃-C₈ cycloalkyl, 3- to 12-membered heterocyclyl, 5- to 10-membered heteroaryl, C₆-C₁₄ aryl, or C₁-C₆ alkyl optionally substituted by deuterium, oxo, —OH or halogen;
   each R^{2a}, R^{2b}, R^{2c}, R^{2e}, and R^{2f} is independently oxo or R^{1a};
   R^{2d} is C₁-C₆ alkyl optionally substituted by R^{2e} or C₃-C₅ cycloalkyl optionally substituted by R^{2f};
   R³ is independently hydrogen, deuterium, C₁-C₆ alkyl, C₂-C₆ alkenyl, C₂-C₆ alkynyl, C₃-C₆ cycloalkyl, C₆-C₁₄ aryl, 5- to 10-membered heteroaryl or 3- to 12-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 10-membered heteroaryl and 3- to 12-membered heterocyclyl of R³ are independently optionally substituted by halogen, deuterium, oxo, —CN, -OR⁸, -NR⁸R⁹, -P(O)(OR⁸)(OR⁹), or C₁-C₆ alkyl optionally substituted by deuterium, halogen, —OH or oxo;
   R⁴ and R⁵ are each independently hydrogen, deuterium, C₁-C₆ alkyl, C₂-C₆ alkenyl, C₂-C₆ alkynyl, C₃-C₆ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 6-membered heteroaryl and 3- to 6-membered heterocyclyl of R⁴ and R⁵ are independently optionally substituted by deuterium, halogen, oxo, —CN, -OR⁸, -NR⁸R⁹ or C₁-C₆ alkyl optionally substituted by deuterium, halogen, —OH or oxo;
   or R⁴ and R⁵ are taken together with the atom to which they attached to form a 3- to 6-membered heterocyclyl optionally substituted by deuterium, halogen, oxo, -OR⁸, -NR⁸R⁹ or
   C₁-C₆ alkyl optionally substituted by deuterium, halogen, oxo or —OH;
   R⁶ and R⁷ are each independently hydrogen, deuterium, C₁-C₆ alkyl optionally substituted by deuterium, halogen, or oxo, C₂-C₆ alkenyl optionally substituted by deuterium, halogen, or
   oxo, or C₂-C₆ alkynyl optionally substituted by deuterium, halogen, or oxo;
   or R⁶ and R⁷ are taken together with the atom to which they attached to form a 3- to 6-membered heterocyclyl optionally substituted by deuterium, halogen, oxo or C₁-C₆ alkyl optionally substituted by deuterium, halogen, or oxo; and
   R⁸ and R⁹ are each independently hydrogen, deuterium, C₁-C₆ alkyl optionally substituted by deuterium, halogen, or oxo, C₂-C₆ alkenyl optionally substituted by deuterium, halogen or
   oxo, or C₂-C₆ alkynyl optionally substituted by deuterium, halogen, or oxo;
   or R⁸ and R⁹ are taken together with the atom to which they attached to form a 3-6 membered heterocyclyl optionally substituted by deuterium, halogen, oxo or C₁-C₆ alkyl optionally substituted by deuterium, oxo, or halogen.

Embodiment C-2. The method of embodiment C-1, wherein in the compound of Formula (II) or a salt thereof, R¹ is 5- to 10-membered heteroaryl optionally substituted by R^{1a}.

Embodiment C-3. The method of embodiment C-1 or embodiment C-2, wherein in the compound of Formula (II), or a salt thereof, R¹ is:
pyrimidinyl, quinazolinyl, pyrazolopyrimidinyl, pyrazinyl, quinolinyl, pyridopyrimidinyl, thienopyrimidinyl, pyridinyl, pyrrolopyrimidinyl, quinoxalinyl, indazolyl, benzothiazolyl, naphthalenyl, purinyl, or isoquinolinyl; and
optionally substituted by deuterium, hydroxy, C₁-C₆ alkyl, C₁-C₆ haloalkyl, C₁-C₆ perhaloalkyl, C₁-C₆ alkoxyl, C₃-C₈ cycloalkyl, C₃-C₈ halocycloalkyl, C₃-C₈ cycloalkoxyl, 5-to 10-membered heteroaryl, C₆-C₁₄ aryl, cyano, amino, alkylamino, or dialkylamino.

Embodiment C-4. The method of any one of embodiments C-1-C-3, wherein in the compound of Formula (II), or a salt thereof, R¹ is:
pyrimidin-2-yl, pyrimidin-4-yl, quinazolin-4-yl, 1H-pyrazolo[3,4-d]pyrimidine-4-yl, 1H-pyrazolo[4,3-d]pyrimidine-7-yl, pyrazin-2-yl, quinoline-4-yl, pyrido[2,3-d]pyrimidin-4-yl, pyrido[3,2-d]pyrimidin-4-yl, pyrido[3,4-d]pyrimidin-4-yl, thieno[2,3-d]pyrimidin-4-yl, thieno[3,2-d]pyrimidin-4-yl, thienopyrimidin-4-yl, pyridin-2-yl, pyridin-3-yl, 7H-pyrrolo[2,3-d]pyrimidin-4-yl, quinoxalin-2-yl, 1H-indazol-3-yl, benzo[d]thiazol-2-yl, naphthalen-1-yl, 9H-purin-6-yl, or isoquinolin-1-yl; and
optionally substituted by: one or more deuterium; methyl; cyclopropyl; fluoro; chloro; bromo; difluoromethyl; trifluoromethyl; methyl and fluoro; methyl and trifluoromethyl; methoxy; cyano; dimethylamino; phenyl; pyridin-3-yl; or pyridin-4-yl.

Embodiment C-5. The method of any one of embodiments C-1-C-4, wherein in the compound of Formula (II), or a salt thereof, R¹ is pyrimidin-4-yl optionally substituted by R^{1a}; or
wherein in the compound of Formula (II), or a salt thereof, R¹ is pyrimidin-4-yl optionally substituted by R^{1a} wherein R^{1a} is 5- to 10-membered heteroaryl or C₁-C₆ alkyl optionally substituted by halogen; or
wherein in the compound of Formula (II), or a salt thereof, R¹ is pyrimidin-4-yl optionally substituted by pyrazolyl, methyl, difluoromethyl, or trifluoromethyl; or
wherein in the compound of Formula (II), or a salt thereof, R¹ is pyrimidin-4-yl substituted by both methyl and trifluoromethyl.

Embodiment C-6. The method of any one of embodiments C-1-C-4, wherein in the compound of Formula (II), or a salt thereof, R¹ is quinazolin-4-yl optionally substituted by R^{1a}; or
wherein in the compound of Formula (II), or a salt thereof, R¹ is quinazolin-4-yl optionally substituted by halogen, C₁-C₆ alkyl optionally substituted by halogen, or C₁-C₆ alkoxy; or
wherein in the compound of Formula (II), or a salt thereof, R¹ is quinazolin-4-yl optionally substituted by fluoro, chloro, methyl, trifluoromethyl or methoxy.

Embodiment C-7. The method of any one of embodiments C-1-C-6, wherein in the compound of Formula (II), or a salt thereof, R² is:
hydrogen;
deuterium;
hydroxy; or
C₁-C₆ alkyl or C₁-C₆ alkoxyl optionally substituted with: deuterium, halogen, C₁-C₆ alkyl, C₁-C₆ haloalkyl, C₁-C₆ hydroxyalkyl, C₁-C₆ alkoxyl, C₃-C₈ cycloalkyl, C₃-C₈ halocycloalkyl, C₃-C₈ cycloalkoxyl, C₆-C₁₄ aryl, C₆-C₁₄ aryloxy, 5- to 10-membered heteroaryl, 5- to 10-membered heteroaryloxy, 3- to 12-membered heterocyclyl optionally substituted with oxo, -C(O)NR⁴R⁵, -NR³C(O)R⁴, or -S(O)₂R³ ; or
   wherein in the compound of Formula (II), or a salt thereof, R² is: methyl, methoxy, ethyl, ethoxy, propyl, cyclopropyl, or cyclobutyl;
each of which is optionally substituted with one or more of: hydroxy, methoxy, ethoxy, acetamide, fluoro, fluoroalkyl, phenoxy, dimethylamide, methylsulfonyl, cyclopropoxyl, pyridin-2-yloxy, optionally methylated or fluorinated pyridine-3-yloxy, N-morpholinyl, N-pyrrolidin-2-onyl, dimethylpyrazol-1-yl, dioxiran-2-yl, morpholin-2-yl, oxetan-3-yl, phenyl, tetrahydrofuran-2-yl, thiazol-2-yl; that is
each of which is substituted with 0, 1, 2, or 3 of deuterium, hydroxy, methyl, fluoro, cyano, or oxo; or
   wherein in the compound of Formula (II), or a salt thereof, R² is C₁-C₆ alkyl optionally substituted by R^{2a} ; or
   wherein in the compound of Formula (II), or a salt thereof, R² is C₁-C₆ alkyl optionally substituted by R^{2a} wherein R^{2a} is: halogen; C₃-C₈ cycloalkyl optionally substituted by halogen; 5- to 10-membered heteroaryl optionally substituted by C₁-C₆ alkyl; -NR⁴R⁵; -NR³C(O)R⁴; -S(O)₂R³; or oxo; or
   wherein in the compound of Formula (II), or a salt thereof, R² is C₁-C₆ alkyl optionally substituted by R^{2a} wherein R^{2a} is: fluoro; cyclobutyl substituted by fluoro; pyrazolyl substituted by methyl; or —S(O)₂CH₃ ; or
   wherein in the compound of Formula (II), or a salt thereof, R² is C₁-C₆ alkyl optionally substituted by -OR³ ; or
   wherein in the compound of Formula (II), or a salt thereof, R² is C₁-C₆ alkyl optionally substituted by -OR³, and R³ is: hydrogen; C₁-C₆ alkyl optionally substituted by halogen; C₃-C₆ cycloalkyl optionally substituted by halogen; C₆-C₁₄ aryl optionally substituted by halogen; or 5- to 6-membered heteroaryl optionally substituted by halogen or C₁-C₆ alkyl; or
   wherein in the compound of Formula (II), or a salt thereof, R² is C₁-C₆ alkyl optionally substituted by -OR³, and R³ is: hydrogen; methyl; ethyl; difluoromethyl; -CH₂CHF₂; —CH₂CF₃; cyclopropyl substituted by fluoro; phenyl optionally substituted by fluoro; or pyridinyl optionally substituted by fluoro or methyl.

Embodiment C-8. The method of any one of embodiments C-1 to C-7, wherein in the compound of Formula (II), or a salt thereof, R² is -CH₂CH₂OCH₃.

Embodiment C-9. The method of any one of embodiments C-1 to C-6, wherein in the compound of Formula (II), or a salt thereof, R² is C₁-C₆ alkyl substituted by both halogen and OR³, wherein R³ is C₁-C₆ alkyl; or
wherein in the compound of Formula (II), or a salt thereof, R² is C₃-C₆ cycloalkyl optionally substituted by R^{2b} ; or
   wherein in the compound of Formula (II), or a salt thereof, R² is cyclopropyl.

Embodiment C-10. The method of any one of embodiments C-1 or C7-C-9, wherein in the compound of Formula (II), or a salt thereof, R¹ is

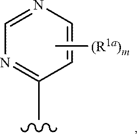

wherein m is 0, 1, 2, or 3 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is

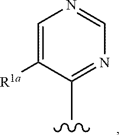

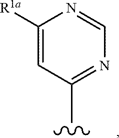

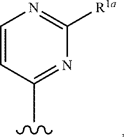

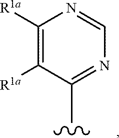

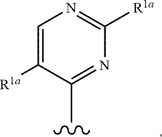

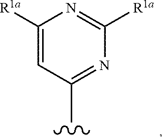

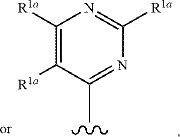

wherein each R^{1a} is independently deuterium, alkyl, haloalkyl, or heteroaryl; or wherein in the compound of Formula (II), or a salt thereof, R¹ is

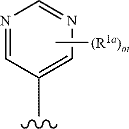

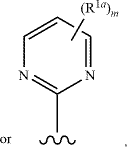

wherein m is 0, 1, 2, or 3 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is,

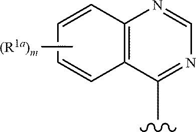

wherein m is 0, 1, 2, 3, 4, or 5 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is

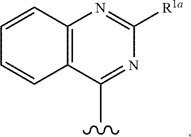

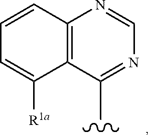

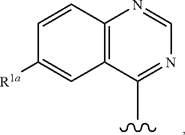

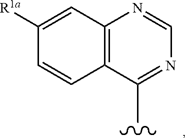

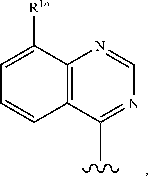

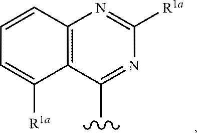

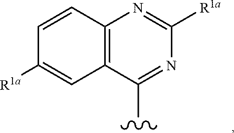

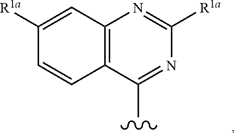

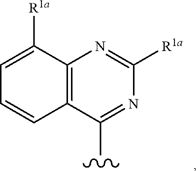

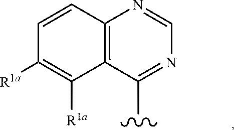

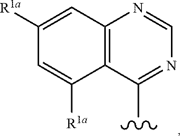

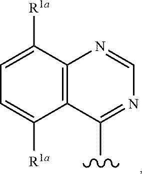

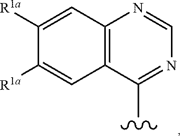

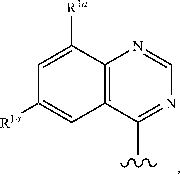

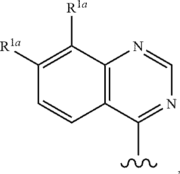

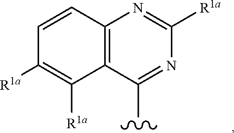

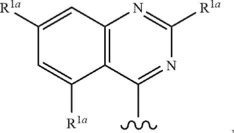

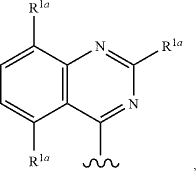

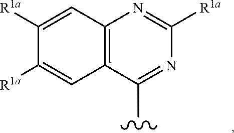

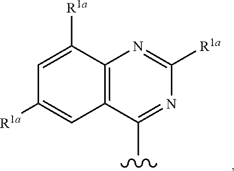

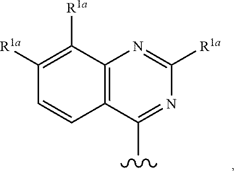

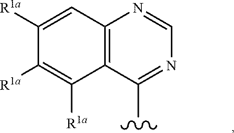

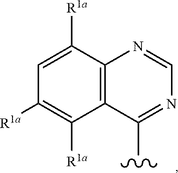

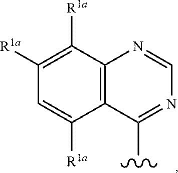

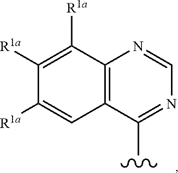

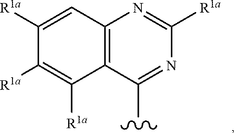

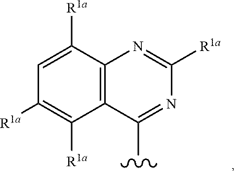

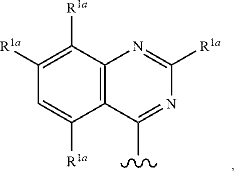

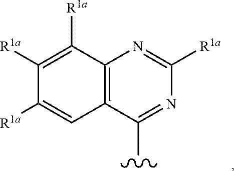

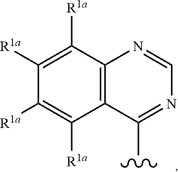

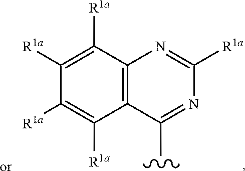

wherein each R^{1a} is independently deuterium, halogen, alkyl, haloalkyl, or alkoxy; or. wherein in the compound of Formula (II), or a salt thereof, R¹ is

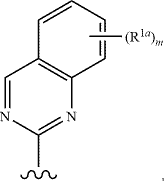

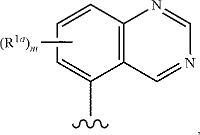

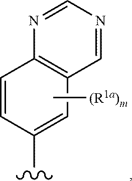

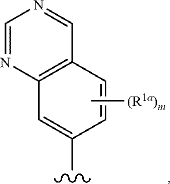

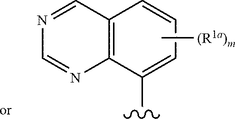

wherein m is 0, 1, 2, 3, 4, or 5 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is,

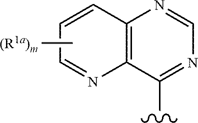

wherein m is 0, 1, 2, 3, or 4, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or. wherein in the compound of Formula (II), or a salt thereof, R¹ is selected from the group consisting of

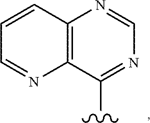

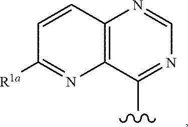

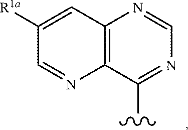

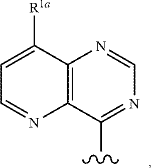

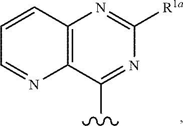

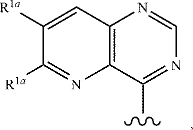

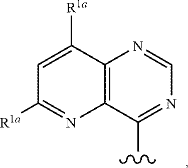

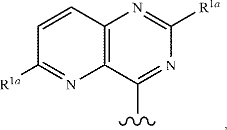

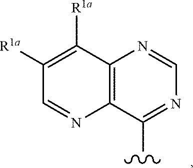

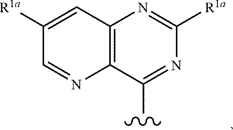

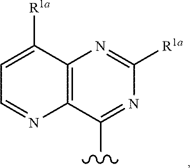

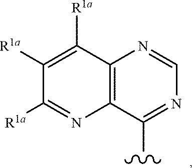

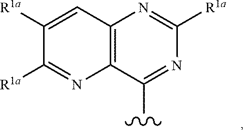

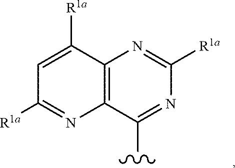

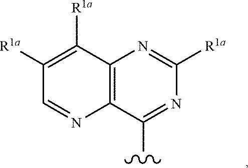

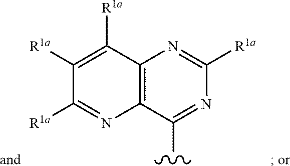

wherein in the compound of Formula (II), or a salt thereof, R¹ is

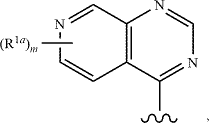

wherein m is 0, 1, 2, 3, or 4, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is selected from the group consisting of

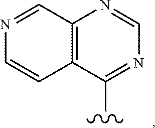

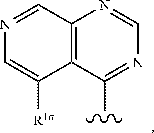

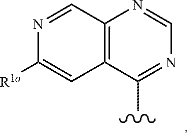

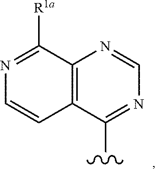

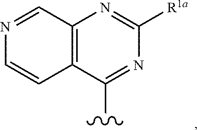

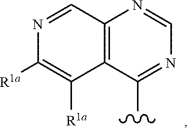

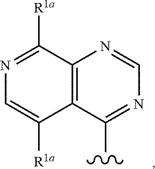

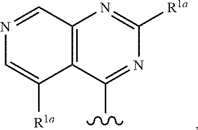

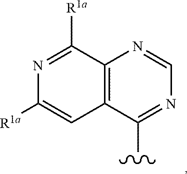

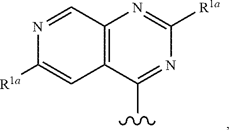

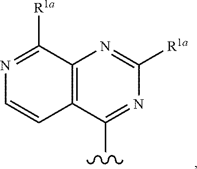

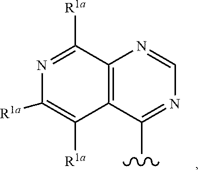

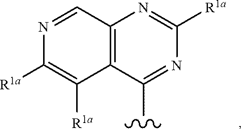

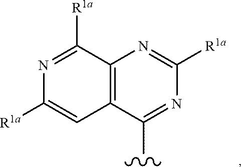

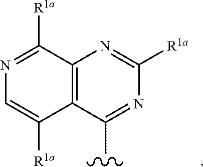

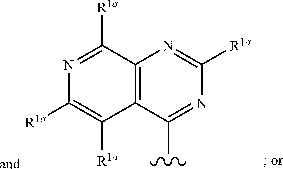

wherein in the compound of Formula (II), or a salt thereof, R¹ is,

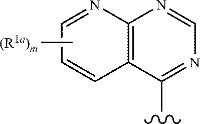

wherein m is 0, 1, 2, 3, or 4, and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is selected from the group consisting of

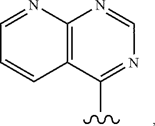

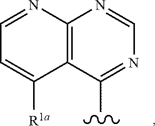

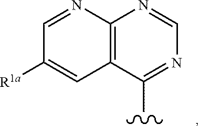

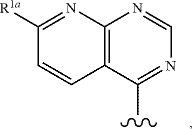

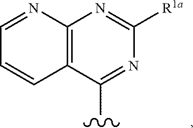

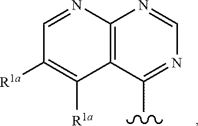

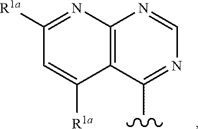

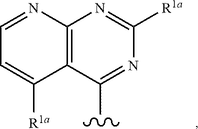

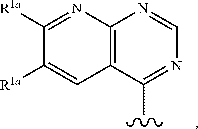

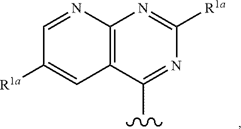

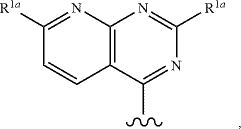

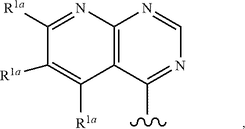

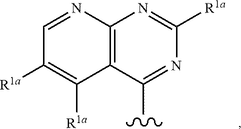

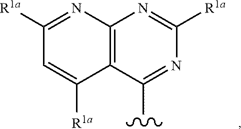

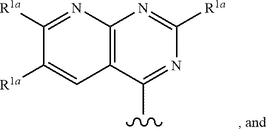

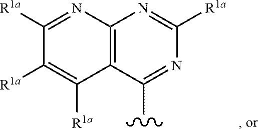

wherein in the compound of Formula (II), or a salt thereof, R¹ is

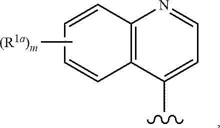

wherein m is 0, 1, 2, 3, 4, 5, or 6 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is

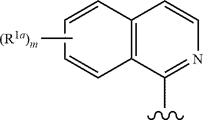

, wherein m is 0, 1, 2, 3, 4, 5, or 6 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is

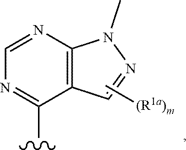

wherein m is 0, 1, or 2 and each R^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is selected from the group consisting of

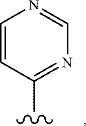

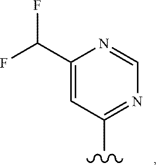

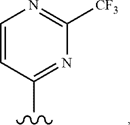

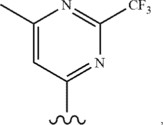

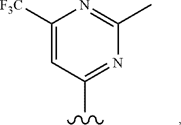

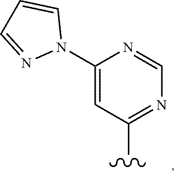

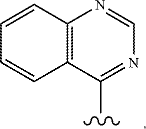

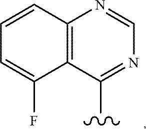

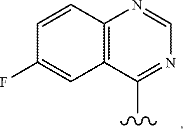

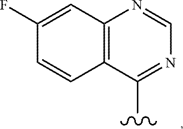

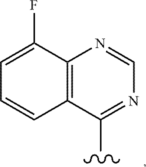

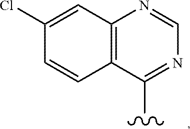

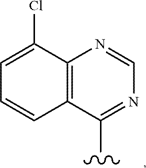

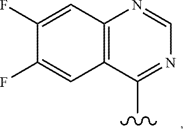

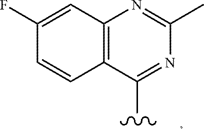

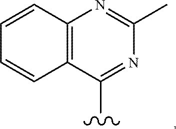

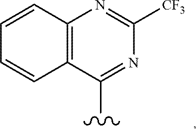

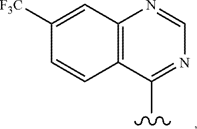

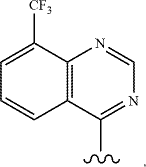

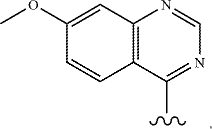

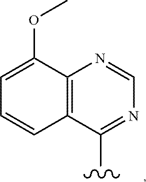

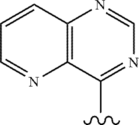

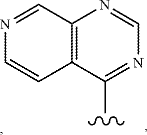

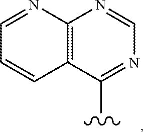

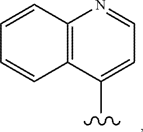

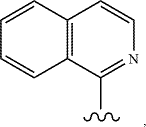

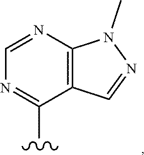

and any of the foregoing groups wherein any one or more hydrogen atom(s) are replaced with deuterium atom(s); or wherein in the compound of Formula (II), or a salt thereof, R¹ is selected from the group consisting of

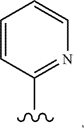

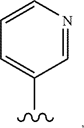

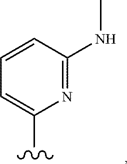

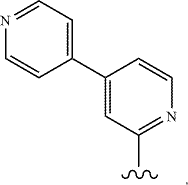

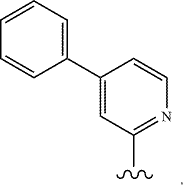

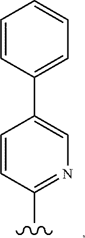

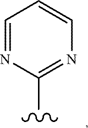

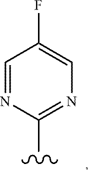

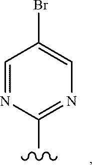

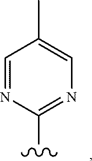

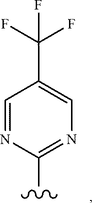

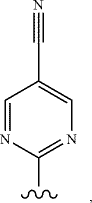

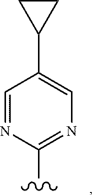

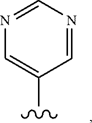

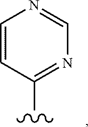

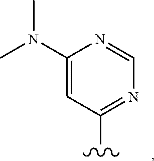

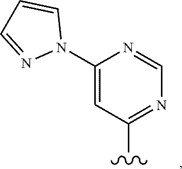

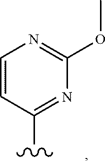

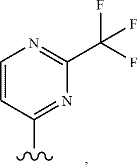

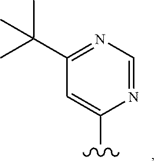

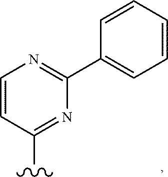

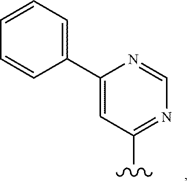

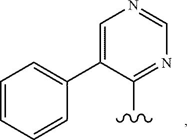

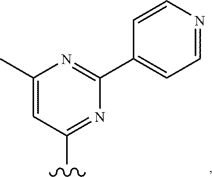

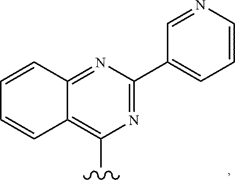

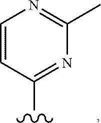

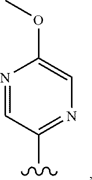

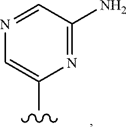

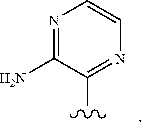

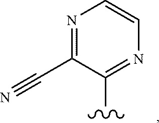

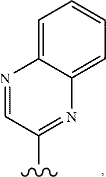

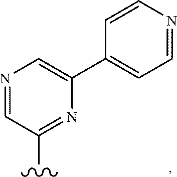

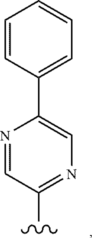

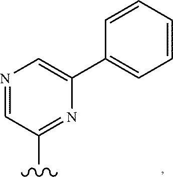

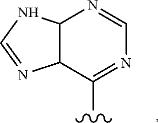

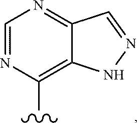

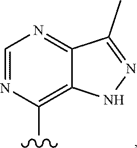

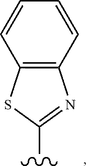

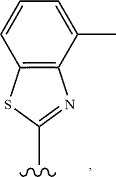

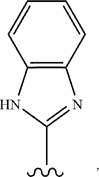

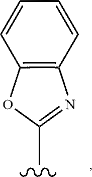

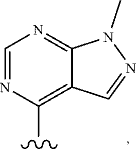

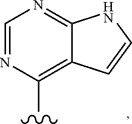

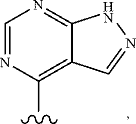

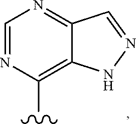

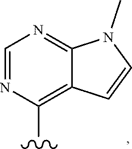

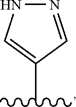

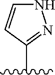 [SMILES: *c1cc[nH]n1]

, and any of the foregoing groups wherein any one or more hydrogen atom(s) are replaced with deuterium atom(s); or wherein in the compound of Formula (II), or a salt thereof, R¹ is selected from the group consisting of

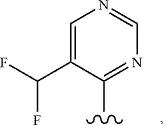

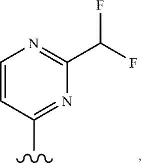

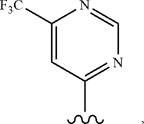

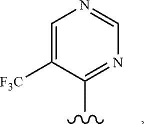

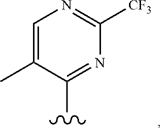

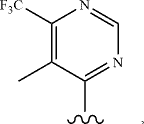

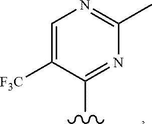

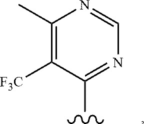

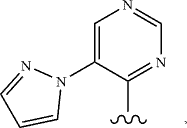

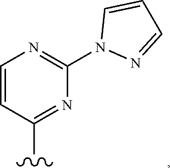

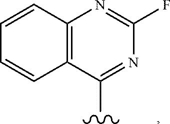

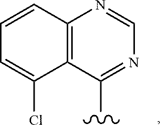

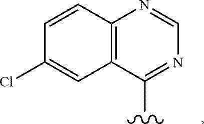

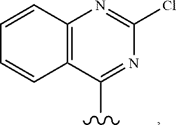

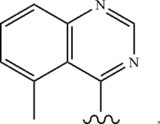

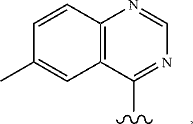

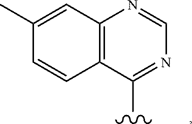

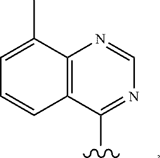

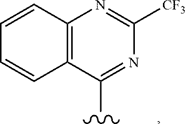

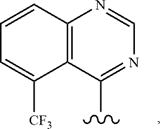

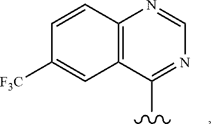

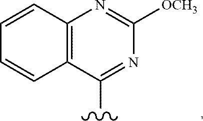

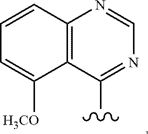

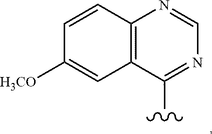

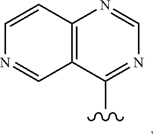

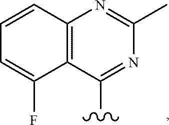

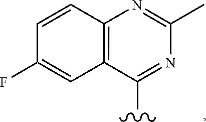

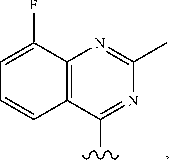

and any of the foregoing groups wherein any one or more hydrogen atom(s) are replaced with deuterium atom(s); or wherein in the compound of Formula (II), or a salt thereof, R¹ is selected from the group consisting of

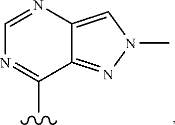

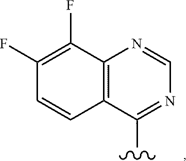

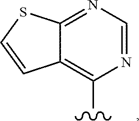

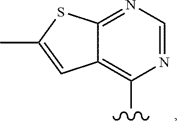

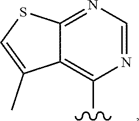

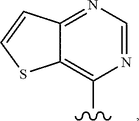

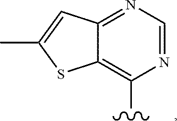

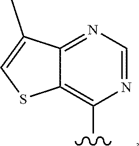

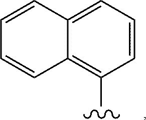

and any of the foregoing groups wherein any one or more hydrogen atom(s) are replaced with deuterium atom(s).

Embodiment C-11. The method of any one of embodiments C-1 to C-6 or C-10, wherein in the compound of Formula (II), or a salt thereof, R² is

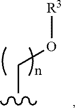

wherein n is 1, 2, 3, 4, 5, or 6, and R³ is C₁-C₂ alkyl optionally substituted by fluoro; phenyl optionally substituted by fluoro; pyridinyl optionally substituted by fluoro or methyl; or cyclopropyl optionally substituted by fluoro; or
wherein in the compound of Formula (II), or a salt thereof, R² is selected from the group consisting of

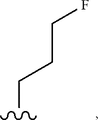

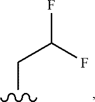

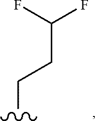

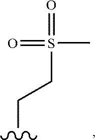

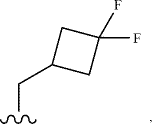

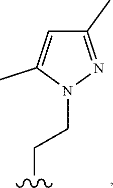

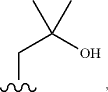

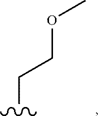

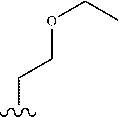

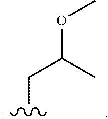

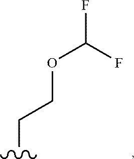

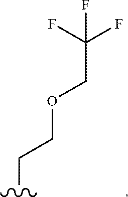

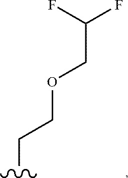

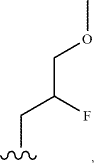

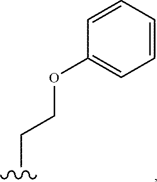

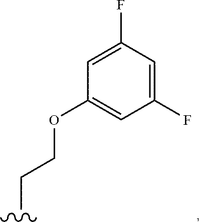

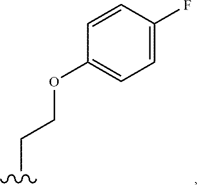

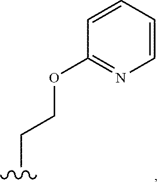

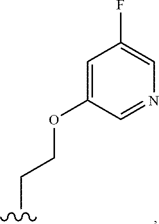

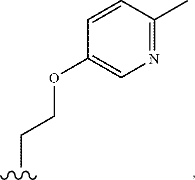

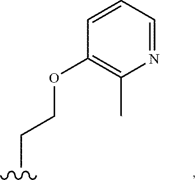

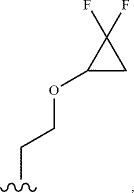

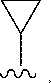

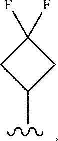

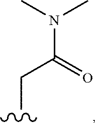

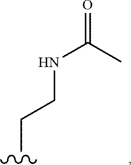

and any of the foregoing groups wherein any one or more hydrogen atom(s) are replaced with deuterium atom(s); or
wherein in the compound of Formula (II), or a salt thereof, R² is selected from the group consisting of

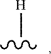

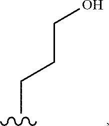

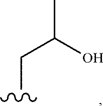

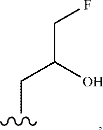

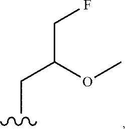

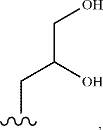

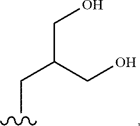

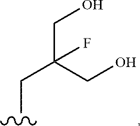

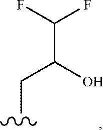

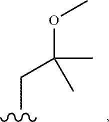

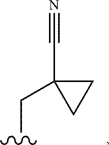

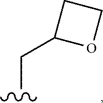

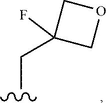

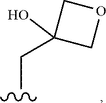

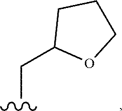

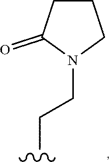

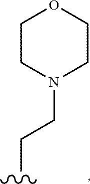

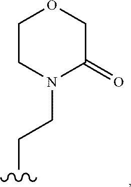

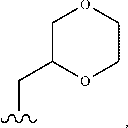

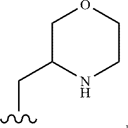

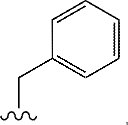

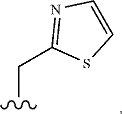

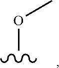

and any of the foregoing groups wherein any one or more hydrogen atom(s) are replaced with deuterium atom(s); or
wherein in the compound of Formula (II), or a salt thereof, R² is C₃-C₅ alkyl substituted by both fluorine and —OCH₃ ; or
wherein in the compound of Formula (II), or a salt thereof, R² is C₁-C₆ alkyl optionally substituted by -OR³, and R³ is phenyl optionally substituted by fluorine; or
wherein in the compound of Formula (II), or a salt thereof, R² is C₁-C₆ alkyl optionally substituted by -OR³, and R³ is pyridinyl optionally substituted by fluorine or methyl; or
wherein in the compound of Formula (II), or a salt thereof, R² is C₁-C₆ alkyl substituted by R^{2a} wherein R^{2a} is halogen; or
wherein in the compound of Formula (II), or a salt thereof, R² is C₁-C₆ alkyl substituted by R^{2a} wherein R^{2a} is deuterium; or
wherein in the compound of Formula (II), or a salt thereof, R² is C₁-C₆ alkyl substituted by R^{2a} wherein R^{2a} is 3- to 12-membered heterocyclyl optionally substituted by oxo; or
wherein in the compound of Formula (II), or a salt thereof, R² is C₁-C₆ alkyl substituted by R^{2a} wherein R^{2a} is 4- to 5-membered heterocyclyl optionally substituted by oxo; or
wherein in the compound of Formula (II), or a salt thereof, R² is C₁-C₆ alkyl substituted by R^{2a} wherein R^{2a} is C₆-C₁₄ aryl optionally substituted by halogen or -OR⁶ ; or
wherein in the compound of Formula (II), or a salt thereof, R² is C₁-C₆ alkyl substituted by R^{2a} wherein R^{2a} is phenyl optionally substituted by halogen or -OR⁶ ; or
wherein in the compound of Formula (II), or a salt thereof, R² is C₁-C₆ alkyl substituted by R^{2a} wherein R^{2a} is 5- to 10-membered heteroaryl optionally substituted by C₁-C₆ alkyl; or
wherein in the compound of Formula (II), or a salt thereof, R² is C₁-C₆ alkyl substituted by R^{2a} wherein R^{2a} is pyrazolyl optionally substituted by methyl; or
wherein in the compound of Formula (II), or a salt thereof, R² is C₁-C₆ alkyl substituted by R^{2a} wherein R^{2a} is C₃-C₈ cycloalkyl optionally substituted by —CN, halogen, or -OR⁶ ; or
wherein in the compound of Formula (II), or a salt thereof, R² is C₁-C₆ alkyl substituted by R^{2a} wherein R^{2a} is -S(O)₂R³ ; or
wherein in the compound of Formula (II), or a salt thereof, R¹ is pyridyl optionally substituted by R^{1a} ; or
wherein in the compound of Formula (II), or a salt thereof, R¹ is indazolyl optionally substituted by R^{1a}; or
wherein in the compound of Formula (II), or a salt thereof, R¹ is 1H-pyrrolopyridyl optionally substituted by R^{1a}; or
wherein in the compound of Formula (II), or a salt thereof, R¹ is quinolinyl optionally substituted by R^{1a}; or
wherein in the compound of Formula (II), or a salt thereof, R¹ is phenyl optionally substituted by R^{1a} ; or
wherein in the compound of Formula (II), or a salt thereof, R¹ is indanyl optionally substituted by R^{1a}.

Embodiment C-12. The method of embodiment C-1, wherein the compound of Formula (II), or a salt thereof, is selected from Compound Nos. 1-66 in FIG. 1.

Embodiment C-13. The method of embodiment C-1, wherein the compound of Formula (II), or a salt thereof, is selected from Compound Nos. 1-147.

Embodiment C-14. The method of embodiment C-1, wherein the compound of Formula (II), or a salt thereof, is selected from Compound Nos. 1-665.

Embodiment C-15. The method of embodiment C-1, wherein the compound of Formula (II), or a salt thereof, is selected from Compound Nos. 1-780.

Embodiment C-16. The method of embodiment C-1, wherein the compound of Formula (II), or a salt thereof, is (S)-4-((2-methoxyethyl)(4-(5,6,7,8-tetrahydro-1,8-naphthyridin-2-yl)butyl)amino)-2-(quinazolin-4-ylamino)butanoic acid:

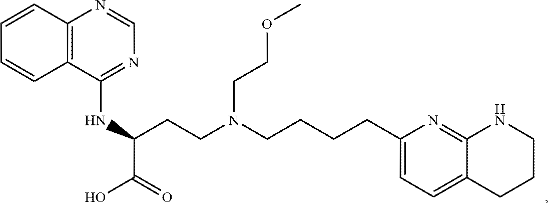

or a salt thereof.

Embodiment C-17. The method of any one of embodiments C-1 to C-16, comprising administering the compound of Formula (II), or a salt thereof, in an amount in milligrams of about 1, 2.5, 5, 7.5, 10, 15, 20, 25, 30, 35, 40, 50, 75, 80, 85, 90, 95, 100, 105, 110, 115, 120, 125, 150, 160, 175, 200, 225, 250, 320, 400, 480, 560, 640, 720, 800, 880, 960, or 1040, or a range between any two of the preceding values.

Embodiment C-18. The method of any one of embodiments C-1 to C-16, comprising administering the compound of Formula (II), or a salt thereof, in an amount effective on administration to the subject to produce a Cₘₐₓ in plasma of the subject in ng/mL of at least about one of 700, 750, 800, 850, 900, 950, 1000, 1050, 1100, 1150, 1200, 1250, 1300, 1350, 1400, 1450, 1500, 1600, 1700, 1800, 1900, 2000, 2100, 2200, 2300, 2400, or 2500, or a range between any two of the preceding concentrations.

Embodiment C-19. The method of any one of embodiments C-1 to C-16, comprising administering the compound of Formula (II), or a salt thereof, in an amount effective on administration to the subject to produce a Cₘₐₓ in plasma of the subject in ng/mL, the Cₘₐₓ corresponding to a plasma-adjusted concentration effective to inhibit a percentage of αvβ₆ or αvβ₁ in the individual of at least about one of 50, 55, 60, 65, 70, 75, 80, 85, 90, 95, or 100, or a range between any two of the preceding percentages.

Embodiment C-20. The method of any one of embodiments C-1 to C-19, comprising administering the compound of Formula (II), or a salt thereof, daily to the subject.

Embodiment C-21. The method of any one of embodiments C-1 to C-19, comprising administering the compound of Formula (II), or a salt thereof, once daily to the subj ect.

Embodiment C-22. The method of any one of embodiments C-1 to C-19, wherein the daily administering is given one time, two times, three times, or four times daily.

Embodiment C-23. The method of any one of embodiments C-20 to C-22, wherein the daily administering is given once daily.

Embodiment C-24. The method of any one of embodiments C-1 to C-23, wherein the disease is a pulmonary disease.

Embodiment C-25. The method of any one of embodiments C-1 to C-23, wherein the disease is a fibrotic disease.

Embodiment C-26. The method of any one of embodiments C-1 to C-23, wherein the disease is a pulmonary fibrotic disease.

Embodiment C-27. The method of any one of embodiments C-1 to C-23, wherein the disease is selected from the group consisting of: idiopathic pulmonary fibrosis, an interstitial lung disease, radiation-induced pulmonary fibrosis, systemic scleroderma or systemic sclerosis associated interstitial lung disease, and nonspecific interstitial pneumonia.

Embodiment C-28. The method of any one of embodiments C-1 to C-23, wherein the disease is idiopathic pulmonary fibrosis.

Embodiment C-29. The method of any one of embodiments C-1 to C-28, wherein the second drug is pirfenidone, represented by:

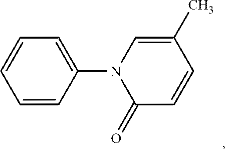

or a salt thereof; or the systematic chemical name 5-methyl-lphenyl-2-1(H)-pyridone, or a salt thereof.

Embodiment C-30. The method of embodiment C-29, wherein the pirfenidone or a salt thereof is orally administered.

Embodiment C-31. The method of embodiment C-30, wherein the pirfenidone or a salt thereof is orally administered to the subject via at least one of a capsule dosage form and a tablet dosage form.

Embodiment C-32. The method of embodiment C-30, wherein the pirfenidone or a salt thereof is orally administered to the subject via the capsule dosage form.

Embodiment C-33. The method of embodiment C-32, wherein the capsule dosage form comprises the pirfenidone or a salt thereof and 1, 2, 3, or 4 ingredients selected from the group consisting of: microcrystalline cellulose, croscarmellose sodium, povidone, and magnesium stearate.

Embodiment C-34. The method of embodiment C-32, wherein at least one of:
the capsule dosage form is characterized by an amount per capsule of the pirfenidone of one of, or about one of: 267 mg, 534 mg, or 801 mg, or a range between any two of the preceding values; or
the amount of pirfenidone orally administered to the subject via the capsule dosage form in a single administration event is one of, or about one of: 267 mg, 534 mg, or 801 mg, or a range between any two of the preceding values.

Embodiment C-35. The method of embodiment C-32, wherein a capsule shell of the capsule dosage form comprises gelatin and titanium dioxide.

Embodiment C-36. The method of embodiment C-30, wherein the pirfenidone or a salt thereof pirfenidone or a salt thereof is orally administered to the subject via the tablet dosage form.

Embodiment C-37. The method of embodiment C-36, wherein the tablet dosage form comprises the pirfenidone or a salt thereof and 1, 2, 3, 4, 5, 6, 7, 8, 9, or 10 ingredients selected from the group consisting of: Microcrystalline cellulose, colloidal anhydrous silica, povidone, croscarmellose sodium, magnesium stearate, polyvinyl alcohol, titanium dioxide, macrogol (polyethylene glycol), talc, and iron oxide.

Embodiment C-38. The method of embodiment C-36, wherein at least one of:
the tablet dosage form is characterized by an amount per capsule of the pirfenidone of one of, or about one of: 267 mg, 534 mg, or 801 mg, or a range between any two of the preceding values; or
the amount of pirfenidone orally administered to the subject via the tablet dosage form in a single administration event is one of, or about one of: 267 mg, 534 mg, or 801 mg, or a range between any two of the preceding values.

Embodiment C-39. The method of embodiment C-36, wherein the tablet dosage form comprises an outer coating.

Embodiment C-40. The method of embodiment C-30, wherein upon initiation of treatment with the pirfenidone or a salt thereof, the pirfenidone is titrated to a full daily dosage over a period of time.

Embodiment C-41. The method of embodiment C-40, wherein upon initiation of treatment with the pirfenidone or a salt thereof, the pirfenidone is titrated to a full daily dosage over a 14-day period as follows:
days 1 through 7, 267 mg three times daily to achieve a daily pirfenidone dosage of 801 mg/day;
days 8 through 14, 534 mg three times daily to achieve a daily pirfenidone dosage of 1602 mg/day; and
days 15 onward, 801 mg three times daily to achieve the full daily pirfenidonedosage of 2403 mg/day.

Embodiment C-42. The method of embodiment C-30, wherein the pirfenidone or a salt thereof is administered in a full daily pirfenidone dosage of 2403 mg/day.

Embodiment C-43. The method of embodiment C-30, wherein the disease is idiopathic pulmonary fibrosis.

Embodiment C-44. The method of embodiment C-30, wherein the pirfenidone is administered as a granulate formulation of 5-methyl-1-phenyl-2-(1H)-pyridone, characterized by one of:
5-methyl-1-phenyl-2-(1H)-pyridone and pharmaceutically acceptable excipients, said excipients comprising an effective amount of binder to increase the AUC of the 5-methyl-1-phenyl-2-(1H)-pyridone at least 45% upon oral administration, as compared to pirfenidone without excipients orally administered in a capsule shell; or
granules comprising 5-methyl-1-phenyl-2-(1H)-pyridone and a glidant, and one or more extragranular excipients comprising an extragranular glidant.

Embodiment C-45. The method of embodiment C-30, wherein the pirfenidone is administered as a coated tablet dosage form comprising a compressed tablet comprising 5-methyl-1-phenyl-2-(1H)-pyridone as an active ingredient; and a coating comprising a light shielding agent disposed on the compressed tablet.

Embodiment C-46. The method of embodiment C-30, wherein the pirfenidone is administered as a capsule dosage form, wherein the capsule dosage form is characterized by one of:
a capsule comprising a pharmaceutical formulation of 5-methyl-1-phenyl-2-(1H)-pyridone, wherein said pharmaceutical formulation comprises 5-30% by weight of pharmaceutically acceptable excipients and 70-95% by weight of 5-methyl-1-phenyl-2-(1H)-pyridone, wherein said excipients comprise an effective amount of binder to increase the AUC of pirfenidone upon oral administration, as compared to a capsule comprising no excipients;
a capsule comprising a pharmaceutical formulation of 5-methyl-1-phenyl-2-(1H)-pyridone, wherein said pharmaceutical formulation comprises 5-methyl-1-phenyl-2-(1H)-pyridone and pharmaceutically acceptable excipients, said excipients comprising an effective amount of binder to increase the AUC of pirfenidone upon oral administration, as compared to a capsule comprising no excipients;
a capsule comprising a pharmaceutical formulation of 5-methyl-1-phenyl-2-(1H)-pyridone, wherein said pharmaceutical formulation comprises pharmaceutically acceptable excipients and 5-methyl-1-phenyl-2-(1H)-pyridone, and the formulation is stable for at least 9 months at 40° C. at 75% relative humidity, as measured by a dissolution of at least 85% of the 5-methyl-1-phenyl-2-(1H)-pyridone after the at least 9 months; or
a capsule comprising a pharmaceutical formulation of 5 methyl-1-phenyl-2-(1H)-pyridone, wherein said pharmaceutical formulation comprises pharmaceutically acceptable excipients and 5-methyl-1-phenyl-2-(1H)-pyridone, and the formulation is stable for at least 18 months at 25° C. at 60% relative humidity, as measured by a dissolution of at least 93% of the 5-methyl-1-phenyl-2-(1H)-pyridone after the at least 18 months.

Embodiment C-47. The method of any one of embodiments C-1 to C-28, wherein the second drug is nintedanib or a salt thereof, and is represented by one or both of:

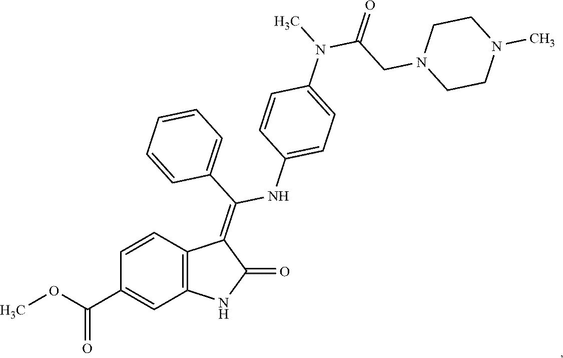

or a salt thereof; or the systematic chemical name 3-Z-[1-(4-(N-((4-methyl-piperazin-1-yl)-methylcarbonyl)-N-methyl-amino)-anilino)-1-phenyl-methylene]-6-methoxycarbonyl-2-indolinone, or a salt thereof.

Embodiment C-48. The method of embodiment C-47, wherein the salt of nintedanib is represented by one or both of:

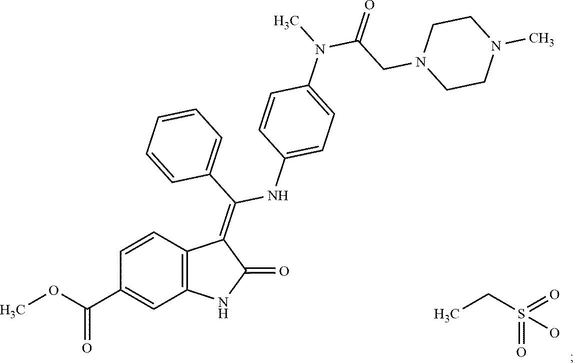

or the systematic chemical name 3-Z-[1-(4-(N-((4-methyl-piperazin-1-yl)-methylcarbonyl)-N-methyl-amino)-anilino)-1-phenyl-methylene|-6-methoxycarbonyl-2-indolinone-monoethanesulphonate.

Embodiment C-49. The method of embodiment C-47 or embodiment C-48, wherein the the nintedanib or a salt thereof is characterized as one or more of:
3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate in crystalline form, having a melting point of Tm.p.=305±5° C. (determined by DSC; evaluation using peak-maximum; heating rate: 10° C./min);
crystalline 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate according to embodiment C-2, the X-ray powder diagram of which includes, inter alia, the characteristic values d=5.43 Å, 5.08 Å, 4.71 Å, 4.50 Å and 4.43 Å with an intensity of more than 40%;
crystalline 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate according to embodiment C-2, characterised by a unit cell determined by X-ray powder diffractometric measurements having the following dimensions: a=16.332 Å, b=19.199 Å, c=11.503 Å, α=95.27°, β=90.13°, y=110.83° and V=3354.4 Å3;
a pharmaceutical composition comprising 3-Z-[1-(4-(N-((4-methyl-piperazin-1-yl)-methylcarbonyl)-N-methyl-amino)-anilino)-1 -phenyl-methylene] -6-methoxy carbonyl-2-indolinone-monoethanesulphonate and one or more inert carriers and/or diluents;
a prodrug of 3-Z-[1-(4-(N-((4-methyl-piperazin-1-yl)-methylcarbonyl)-N-methyl-amino)-anilino)-1 -phenyl-methylene] -6-methoxycarbonyl-2-indolinone-monoethanesulphonate; or
3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate in crystalline form.

Embodiment C-50. The method of any one of embodiments C-47 to C-49, wherein the nintedanib or salt thereof is orally administered.

Embodiment C-51. The method of any one of embodiments C-47 to C-50, wherein the nintedanib or a salt thereof is orally administered to the subject via at least one of a lipid dosage form and a capsule dosage form.

Embodiment C-52. The method of embodiment C-51, wherein at least one of:
the lipid dosage form is characterized by an amount of the nintedanib or salt thereof equivalent to, or equivalent to about, 100 mg or 150 mg of nintedanib, or a range between about 100 mg to about 150 mg of nintedanib; or
the amount of nintedanib or salt thereof orally administered to the subject via the lipid dosage form in a single administration event is equivalent to, or equivalent to about, 100 mg or 150 mg of nintedanib, or a range between about 100 mg to about 150 mg of nintedanib.

Embodiment C-53. The method of embodiment C-51, wherein at least one of:
the lipid dosage form is characterized by an amount of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib; or
the amount of nintedanib or salt thereof orally administered to the subject via the lipid dosage form in a single administration event is characterized by an amount per capsule of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib.

Embodiment C-54. The method of embodiment C-51, wherein the nintedanib or a salt thereof is orally administered to the subject via the lipid dosage form, the lipid dosage form characterized by one or more of:
(a) a formulation of the active substance 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate which comprises a lipid suspension of the active substance in 1 to 90 wt. % of medium chain triglycerides, 1 to 30 wt. % of hard fat and 0.1 to 10 wt. % of lecithin;

(b) a pharmaceutical dosage form which is a viscous lipid suspension formulation comprising:
   10 to 50 wt. % of the active substance 3-Z-[1-(4-(N-((4-methyl-piperazin-1-yl)-
   methylcarbonyl)-N-methyl-amino)-anilino)-1-phenyl-methylenel]-6-methoxycarbonyl-2-indolinone-monoethanesulphonate,
   10 to 70 wt. % of medium chain triglycerides;
   10 to 30 wt. % of hard fat; and
   0.25 to 2.5 wt. % of lecithin,
   which delivers an immediate release profile in which not less than 70% (Q65%) of the active substance is dissolved in 60 minutes in vitro under the following in vitro dissolution conditions according to European Pharmacopeia 6.2: Apparatus 2 (paddle), dissolution medium with 0.1 M HCl (pH 1) and stirring speed of 50 to 150 rpm, at a temperature of 37° C.; or
(c) a lipid suspension comprising, consisting of, or consisting essentially of 3-Z-[1-(4-(N-((4-methyl-piperazin-1 -yl)-methylcarbonyl)-N-methyl-amino)-anilino)-1-phenyl-methylene]-6-methoxycarbonyl-2-indolinone-monoethanesulphonate, medium chain triglycerides, hard fat and lecithin, wherein the medium chain triglycerides, hard fat and lecithin are present in the lipid suspension in the following amounts:
   1 to 90 wt. % of medium chain triglycerides,
   1 to 30 wt. % of hard fat, and
   0.1 to 10 wt. % of lecithin.

Embodiment C-55. The method of embodiment C-51, wherein the nintedanib or a salt thereof is orally administered to the subject via the capsule dosage form, the capsule dosage form comprising a capsule shell and a capsule formulation.

Embodiment C-56. The method of embodiment C-51, wherein the nintedanib or a salt thereof is orally administered to the subject via the capsule dosage form, the capsule dosage form comprising a capsule shell and a capsule formulation, the capsule formulation comprising the lipid dosage form characterized by one or more of:
(a) a formulation of the active substance 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate which comprises a lipid suspension of the active substance in 1 to 90 wt. % of medium chain triglycerides, 1 to 30 wt. % of hard fat and 0.1 to 10 wt. % of lecithin;

(b) a pharmaceutical dosage form which is a viscous lipid suspension formulation comprising:
   10 to 50 wt. % of the active substance 3-Z-[1-(4-(N-((4-methyl-piperazin-1-yl)-methylcarbonyl)-N-methyl-amino)-anilino)-1-phenyl-methylenel]-6-methoxycarbonyl-2-indolinone-monoethanesulphonate,
   10 to 70 wt. % of medium chain triglycerides;
   10 to 30 wt. % of hard fat; and
   0.25 to 2.5 wt. % of lecithin,
   which delivers an immediate release profile in which not less than 70% (Q65%) of the active substance is dissolved in 60 minutes in vitro under the following in vitro dissolution conditions according to European Pharmacopeia 6.2: Apparatus 2 (paddle), dissolution medium with 0.1 M HCl (pH 1) and stirring speed of 50 to 150 rpm, at a temperature of 37° C.; or
(c) a lipid suspension comprising, consisting of, or consisting essentially of 3-Z-[1-(4-(N-((4-methyl-piperazin-1 -yl)-methylcarbonyl)-N-methyl-amino)-anilino)-1-phenyl-methylene]-6-methoxycarbonyl-2-indolinone-monoethanesulphonate, medium chain triglycerides, hard fat and lecithin, wherein the medium chain triglycerides, hard fat and lecithin are present in the lipid suspension in the following amounts:
   1 to 90 wt. % of medium chain triglycerides,
   1 to 30 wt. % of hard fat, and
   0.1 to 10 wt. % of lecithin.

Embodiment C-57. The method of embodiment C-55, wherein at least one of:
the capsule dosage form is characterized by an amount per capsule of the nintedanib or salt thereof equivalent to, or equivalent to about, 100 mg or 150 mg of nintedanib, or a range between about 100 mg to about 150 mg of nintedanib; or.
the amount of nintedanib or salt thereof orally administered to the subject via the capsule dosage form in a single administration event is equivalent to, or equivalent to about, 100 mg or 150 mg of nintedanib, or a range between about 100 mg to about 150 mg of nintedanib.

Embodiment C-58. The method of embodiment C-55, wherein at least one of:
the capsule dosage form is characterized by an amount per capsule of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib; or.
the amount of nintedanib or salt thereof orally administered to the subject via the capsule dosage form in a single administration event is characterized by an amount per capsule of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib.

Embodiment C-59. The method of embodiment C-55, wherein the capsule shell of the capsule dosage form comprises 1, 2, 3, 4, 5, or 6 of: gelatin, glycerol, titanium dioxide, red ferric oxide, yellow ferric oxide, and black ink.

Embodiment C-60. The method of any one of embodiments C-1 to C-28 or C-47 to C-59, wherein the second drug is nintedanib or a salt thereof and is administered to the subject twice daily in a dosage of the nintedanib or a salt thereof equivalent to, or equivalent to about, 100 mg of nintedanib, for a total daily dose equivalent to, or equivalent to about, 200 mg of nintedanib.

Embodiment C-61. The method of embodiment C-60, wherein the subject has one of a mild hepatic impairment or a side effect associated with nintedanib or a salt thereof.

Embodiment C-62. The method of any one of embodiments C-1 to C-28 or C-47 to C-59, wherein the second drug is nintedanib or a salt thereof and is administered to the subject twice daily in a dosage of the nintedanib or a salt thereof equivalent to, or equivalent to about, 150 mg of nintedanib, for a total daily dose equivalent to, or equivalent to about, 300 mg of nintedanib.

Embodiment C-63. The method of any of embodiments C-47 to C-62, wherein the disease is selected from the group consisting of idiopathic pulmonary fibrosis, an interstitial lung disease, and systemic sclerosis-associated interstitial lung disease.

Embodiment C-64. The method of any of embodiments C-47 to C-62, wherein the disease is idiopathic pulmonary fibrosis.

Embodiment C-65. The method of embodiment C-63, wherein the interstitial lung disease includes chronic fibrosing interstitial lung diseases (ILDs) with a progressive phenotype.

Embodiment C-66. The method of embodiment C-63, wherein the disease includes systemic sclerosis-associated interstitial lung disease, and treating the subject includes slowing the rate of decline in pulmonary function in the subject associated with the systemic sclerosis-associated interstitial lung disease.

Embodiment C-67. The method of any one of embodiments C-1 to C-66, comprising administering the first drug to the subject in an amount effective to modulate at least one integrin in the subject.

Embodiment C-68. The method of any one of embodiments C-1 to C-66, wherein the subject has at least one tissue in need of therapy and the tissue has at least one elevated level of:
at least one integrin activity and/or expression;
a pSMAD/SMAD value;
new collagen formation or accumulation;
total collagen; and
Type I Collagen gene Col1a1 expression;
and wherein the level is elevated compared to a healthy state of the tissue.

Embodiment C-69. The method of embodiment C-67 or embodiment C-68, comprising administering the first drug to the subject in an amount effective to inhibit the at least one integrin in the subject.

Embodiment C-70. The method of embodiment C-67 or embodiment C-68, wherein the at least one integrin in the subject comprises αv.

Embodiment C-71. The method of embodiment C-67 or embodiment C-68, wherein the at least one integrin in the subject is selected from the group consisting of αvβ₆ integrin and αvβ₁ integrin.

Embodiment C-72. The method of embodiment C-67 or embodiment C-68, wherein the at least one integrin in the subject comprises both αvβ₆ integrin and αvβ₁ integrin.

Embodiment C-73. The method of embodiment C-67 or embodiment C-68, comprising administering the first drug to the subject in an amount effective to inhibit one or both of αvβ₁ integrin and αvβ₆ integrin in the subject.

Embodiment C-74. The method of any of embodiments C-67 to C-73, wherein the method selectively reduces αvβ₁ integrin activity and/or expression compared to αvβ₆ integrin activity and/or expression in the subject.

Embodiment C-75. The method of any of embodiments C-67 to C-73, wherein the method selectively reduces αvβ₆ integrin activity and/or expression compared to αvβ₁ integrin activity and/or expression in the subject.

Embodiment C-76. The method of any of embodiments C-67 to C-73, wherein the method reduces both αvβ₁ integrin and αvβ₆ integrin activity and/or expression compared to at least one other αv-containing integrin in the subject.

Embodiment C-77. The method of any of embodiments C-67 to C-73, wherein the activity of αvβ₁ integrin in one or more fibroblasts is reduced in the subject.

Embodiment C-78. The method of any of embodiments C-67 to C-73, wherein the activity of αvβ₆ integrin in one or more epithelial cells is reduced in the subject.

Embodiment C-79. The method of embodiment C-68, wherein the at least one tissue in the subject comprises one or more of: lung tissue, liver tissue, skin tissue, cardiac tissue, kidney tissue, gastrointestinal tissue, gall bladder tissue, and bile duct tissue.

Embodiment C-80. The method of any one of embodiments C-68 to C-79, wherein the tissue has an elevated pSMAD2/SMAD2 value or an elevated pSMAD3/SMAD3 value compared to the healthy state of the tissue.

Embodiment C-81. The method of any one of embodiments C-1 to C-80, wherein the first drug and/or the second drug are administered orally to the subject.

Embodiment C-82. The method of any one of embodiments C-1 to C-81, wherein the first drug and/or the second drug are administered to the subject with food.

Embodiment C-83. The method of any one of embodiments C-1 to C-82, wherein the first drug and the second drug are administered to the subject at the same time or on a same schedule.

Embodiment C-84. The method of any one of embodiments C-1 to C-82, wherein the first drug and the second drug are administered to the subject at different times or on a different schedule.

Embodiment C-85. The method of any one of embodiments C-1 to C-82, wherein the second drug is administered to the subject over a period of days, weeks, months, or years before first administering the first drug to the subject.

Embodiment C-86. The method of any one of embodiments C-1 to C-85, wherein after administering the first and second drugs to the subject over a period of days, weeks, months, or years, the dose of the second drug is decreased in amount or frequency.

Embodiment C-87. The method of any one of embodiments C-1 to C-85, wherein after administering the first and second drugs to the subject over a period of days, weeks, months, or years, administration of the second drug is discontinued.

Embodiment C-88. The method of embodiment C-86 or embodiment C-87, wherein the second drug is decreased in amount or frequency or discontinued after the subject experiences a stabilization, improvement, or remission in the disease.

Embodiment C-89. The method of any of embodiments C-1 to C-88, wherein the subject is human.

Embodiment C-90. A method of amelioration of decline of forced vital capacity (FVC) in a subject in need thereof, comprising administering to the subject a compound of formula (II):

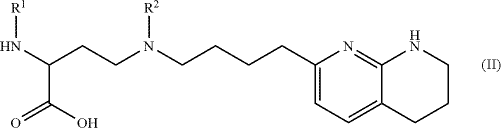

or a salt thereof, whereby the subject is treated for the disease; wherein in the compound of Formula (II):
R¹ is C₆-C₁₄ aryl or 5- to 10-membered heteroaryl wherein the C₆-C₁₄ aryl and 5- to 10-membered heteroaryl are optionally substituted by R^{1a};
R² is hydrogen; deuterium; C₁-C₆ alkyl optionally substituted by R^{2a}; —OH; —O—C₁-C₆ alkyl optionally substituted by R^{2a}; C₃-C₆ cycloalkyl optionally substituted by R^{2b}; —O—C₃-C₆ cycloalkyl optionally substituted by R^{2b}; 3- to 12-membered heterocyclyl optionally substituted by R^{2c}; or -S(O)₂R^{2d}; with the proviso that any carbon atom bonded directly to a nitrogen atom is optionally substituted with an R^{2a} moiety other than halogen; each R^{1a} is independently C₁-C₆ alkyl, C₂-C₆ alkenyl, C₂-C₆ alkynyl, C₃-C₈ cycloalkyl, C₄-Cs cycloalkenyl, 3- to 12-membered heterocyclyl, 5- to 10-membered heteroaryl, C₆-C₁₄ aryl, deuterium, halogen, —CN, -OR³, -SR³, -NR⁴R⁵, —NO₂, -C=NH(OR³), -C(O)R³, -OC(O)R³,
-C(O)OR³, -C(O)NR⁴R⁵, -NR³C(O)R⁴, -NR³C(O)OR⁴, -NR³C(O)NR⁴R⁵, -S(O)R³, -S(O)₂R³,
-NR³S(O)R⁴, -NR³S(O)₂R⁴, -S(O)NR⁴R⁵, -S(O)₂NR⁴R⁵, or -P(O)(OR⁴)(OR⁵), wherein each R^{1a}is, where possible, independently optionally substituted by deuterium, halogen, oxo,
-OR⁶, -NR⁶R⁷, -C(O)R⁶, —CN, -S(O)R⁶, -S(O)₂R⁶, -P(O)(OR⁶)(OR⁷), C₃-C₈ cycloalkyl, 3- to 12-membered heterocyclyl, 5- to 10-membered heteroaryl, C₆-C₁₄ aryl, or C₁-C₆ alkyl optionally substituted by deuterium, oxo, —OH or halogen;
each R^{2a}, R^{2b}, R^{2c}, R^{2e}, and R^{2f} is independently oxo or R^{1a};
R^{2d} is C₁-C₆ alkyl optionally substituted by R^{2e} or C₃-C₅ cycloalkyl optionally substituted by R^{2f}_{;}
R³ is independently hydrogen, deuterium, C₁-C₆ alkyl, C₂-C₆ alkenyl, C₂-C₆ alkynyl, C₃-C₆ cycloalkyl, C₆-C₁₄ aryl, 5- to 10-membered heteroaryl or 3- to 12-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 10-membered heteroaryl and 3- to 12-membered heterocyclyl of R³ are independently optionally substituted by halogen, deuterium, oxo, —CN, -OR⁸, -NR⁸R⁹, -P(O)(OR⁸)(OR⁹), or
C₁-C₆ alkyl optionally substituted by deuterium, halogen, —OH or oxo;
R⁴ and R⁵ are each independently hydrogen, deuterium, C₁-C₆ alkyl, C₂-C₆ alkenyl, C₂-C₆ alkynyl, C₃-C₆ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 6-membered heteroaryl and 3- to 6-membered heterocyclyl of R⁴ and R⁵ are independently optionally substituted by deuterium, halogen, oxo, —CN, -OR⁸, -NR⁸R⁹ or Ci-C₆ alkyl optionally substituted by deuterium, halogen, —OH or oxo;
or R⁴ and R⁵ are taken together with the atom to which they attached to form a 3- to 6-membered heterocyclyl optionally substituted by deuterium, halogen, oxo, -OR⁸, -NR⁸R⁹ or
C₁-C₆ alkyl optionally substituted by deuterium, halogen, oxo or —OH;
R⁶ and R⁷ are each independently hydrogen, deuterium, C₁-C₆ alkyl optionally substituted by deuterium, halogen, or oxo, C₂-C₆ alkenyl optionally substituted by deuterium, halogen, or
oxo, or C₂-C₆ alkynyl optionally substituted by deuterium, halogen, or oxo;
or R⁶ and R⁷ are taken together with the atom to which they attached to form a 3- to 6-membered heterocyclyl optionally substituted by deuterium, halogen, oxo or C₁-C₆ alkyl optionally substituted by deuterium, halogen, or oxo; and
R⁸ and R⁹ are each independently hydrogen, deuterium, C₁-C₆ alkyl optionally substituted by deuterium, halogen, or oxo, C₂-C₆ alkenyl optionally substituted by deuterium, halogen or
oxo, or C₂-C₆ alkynyl optionally substituted by deuterium, halogen, or oxo;
or R⁸ and R⁹ are taken together with the atom to which they attached to form a 3-6 membered heterocyclyl optionally substituted by deuterium, halogen, oxo or C₁-C₆ alkyl optionally substituted by deuterium, oxo, or halogen; or
comprising administering to the subject a compound selected from Compounds 1-780, or a pharmaceutically acceptable salt thereof..

Embodiment C-91. The method of embodiment C-90, wherein the compound of Formula (II) is (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof

Embodiment C-92. The method of embodiment C-91, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in a therapeutically effective amount sufficient to reduce the decline in FVC in the subject as compared to a subject who has not been administered (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof.

Embodiment C-93. The method of embodiment C-90 or embodiment C-91, wherein the administering is for at least about 12 weeks.

Embodiment C-94. The method of any one of embodiments C-90 to C-93, wherein the administering is for about a 12 week period.

Embodiment C-95. The method of any one of embodiments C-90 to C-94, wherein the administering is daily.

Embodiment C-96. The method of any one of embodiments C-90 to C-95, wherein the administering is once daily.

Embodiment C-97. The method of any one of embodiments C-90 to C-96, wherein the amelioration of decline in FVC is a reduction in decline of FVC.

Embodiment C-98. The method of embodiment C-97, wherein the reduction in decline in FVC is about 50 mL or less.

Embodiment C-99. The method of embodiment C-97, wherein the reduction in decline in FVC is about 30 mL or less.

Embodiment C-100. The method of embodiment C-97, wherein the reduction in decline in FVC is about 15 mL or less.

Embodiment C-101. The method of any one of embodiments C-97 to C-100, wherein the administering is for about a 12 week period and the decline in FVC is about 50 mL or less from the start of the period to the end of the period.

Embodiment C-102. The method of any one of embodiments C-97 to C-100, wherein the decline in FVC is about 30 mL or less from the start of the period to the end of the period.

Embodiment C-103. The method of any one of embodiments C-97 to C-100, wherein the decline in FVC is about 15 mL or less from the start of the period to the end of the period.

Embodiment C-104. The method of any of embodiments C-97 to C-103, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 40 mg daily, or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment C-105. The method of any of embodiments C-97 to C-103, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 80 mg daily, or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment C-106. The method of any of embodiments C-97 to C-103 wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 160 mg daily, or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment C-107. The method of any of embodiments C-97 to C-103, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of at least about 700 ng/mL.

Embodiment C-108. The method of any of embodiments C-97 to C-103, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,000 ng/mL plus or minus 200 ng/mL.

Embodiment C-109. The method of any of embodiments C-97 to C-103, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,600 ng/mL plus or minus 300 ng/mL.

Embodiment C-110. The method of any of embodiments C-97 to C-103, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 2,700 ng/mL plus or minus 400 ng/mL.

Embodiment C-111. The method of any one of embodiments C-90 to C-96, wherein the amelioration of decline in FVC is an increase of FVC.

Embodiment C-112. The method of embodiment C-111, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in a therapeutically effective amount sufficient to increase FVC in the subject as compared to a subject who has not been administered (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof.

Embodiment C-113. The method of embodiment C-111 or embodiment C-112, wherein the administering is for at least about 12 weeks.

Embodiment C-114. The method of embodiment C-111 or embodiment C-112, wherein the administering is for about a 12 week period.

Embodiment C-115. The method of any one of embodiments C-111 to C-114, wherein the administering is daily.

Embodiment C-116. The method of any one of embodiments C-111 to C-114, wherein the administering is once daily.

Embodiment C-117. The method of any one of embodiments C-111 to C-116, wherein the increase in FVC is about 10 mL or more, about 20 mL or more, about 30 mL or more, about 40 mL or more, about 50 mL or more, or about 60 mL or more.

Embodiment C-118. The method of any one of embodiments C-111 to C-116, wherein the increase in FVC is about 70 mL or more, about 80 mL or more, about 90 mL or more, about 100 mL or more, about 110 mL or more, or about 120 mL or more.

Embodiment C-119. The method of any one of embodiments C-111 to C-116, wherein the increase in FVC is about 130 mL or more, about 140 mL or more, about 150 mL or more, about 160 mL or more, about 170 mL or more, about 180 mL or more, or about 185 mL or more.

Embodiment C-120. The method of any one of embodiments C-111 to C-116, wherein the administering is for about a 12 week period and the increase in FVC is about 10 mL or more, about 20 mL or more, about 30 mL or more, about 40 mL or more, about 50 mL or more, or about 60 mL or more from the start of the period to the end of the period.

Embodiment C-121. The method of any one of embodiments C-111 to C-116, wherein the increase in FVC is about 70 mL or more, about 80 mL or more, about 90 mL or more, about 100 mL or more, about 110 mL or more, or about 120 mL or more from the start of the period to the end of the period.

Embodiment C-122. The method of any one of embodiments C-111 to C-116, wherein the increase in FVC is about 130 mL or more, about 140 mL or more, about 150 mL or more, about 160 mL or more, about 170 mL or more, about 180 mL or more, or about 185 mL or more from the start of the period to the end of the period.

Embodiment C-123. The method of any one of embodiments C-111 to C-122, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 40 mg daily, or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment C-124. The method of any one of embodiments C-111 to C-122, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 80 mg daily, or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment C-125. The method of any one of embodiments C-111 to C-122, wherein the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 160 mg daily, or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid daily.

Embodiment C-126. The method of any one of embodiments C-111 to C-122, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of at least about 700 ng/mL.

Embodiment C-127. The method of any one of embodiments C-111 to C-122, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,000 ng/mL plus or minus 200 ng/mL.

Embodiment C-128. The method of any one of embodiments C-111 to C-122, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,600 ng/mL plus or minus 300 ng/mL.

Embodiment C-129. The method of any one of embodiments C-111 to C-122, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 2,700 ng/mL plus or minus 400 ng/mL.

Embodiment C-130. The method of any one of embodiments C-90 to C-129, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in a therapeutically effective amount.

Embodiment C-131. The method of any one of embodiments C-90 to C-130, wherein the pharmaceutically acceptable salt is a phosphate salt of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment C-132. The method of embodiment C-131, wherein the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid is crystalline.

Embodiment C-133. The method of any one of embodiments C-90 to C-132, wherein the subject has a fibrotic lung disease.

Embodiment C-134. The method of embodiment C-133, wherein the fibrotic lung disease is idiopathic pulmonary fibrosis (IPF).

Embodiment C-135. The method of any one of embodiments C-90 to C-134, wherein the subject is a human.

Embodiment C-136. The method of any one of embodiments C-90 to C-135, wherein the subject is concurrently being treated with a standard medical therapy or a standard of care.

Embodiment C-137. The method of embodiment C-136, wherein the standard medical therapy or standard of care comprises administration of pirfenidone, administration of nintedanib, or administration of pirfenidone and nintedanib.

Embodiment C-138. The method of any one of embodiments C-90 to C-135, wherein the subject has not been previously treated with a standard medical therapy or a standard of care for a lung disorder.

Embodiment C-139. The method of embodiment C-138, wherein the standard medical therapy or standard of care comprises administration of pirfenidone, administration of nintedanib, or administration of pirfenidone and nintedanib.

Embodiment C-140. The method of any one of embodiments C-90 to C-135 or C-138 to C-139, wherein the subject is not being concurrently treated with a standard medical therapy or a standard of care.

Embodiment C-141. The method of embodiment C-140, wherein the standard medical therapy or standard of care comprises administration of pirfenidone, administration of nintedanib, or administration of pirfenidone and nintedanib.

Embodiment C-142. The method of any one of embodiments C-90 to C-135 or C-138 to C-141, wherein the subject is not administered any treatment for a lung disorder other than (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof.

Embodiment C-143. The method of any one of embodiments C-90 to C-142, wherein the method is not accompanied by a serious adverse event.

Embodiment C-144. The method of any one of embodiments C-90 to C-142, wherein the probability of a serious adverse event is less than about 20%.

Embodiment C-145. The method of embodiment C-143 or embodiment C-144, wherein the serious adverse event is a gastrointestinal adverse event.

Embodiment C-146. The method of any one of embodiments C-90 to C-142, wherein the incidence of adverse events is lower than the incidence of adverse events for a standard medical therapy or a standard of care.

Embodiment C-147. The method of embodiment C-146, wherein the standard medical therapy or standard of care comprises administration of pirfenidone, administration of nintedanib, or administration of pirfenidone and nintedanib.

Embodiment C-148. The method of embodiment C-146 or embodiment C-147, wherein the adverse events are gastrointestinal adverse events.

Embodiment C-149. A method of modulating αvβ₆ integrin, αvβ₁ integrin, or both αvβ₆ integrin and αvβ₁ integrin in a subject in need thereof, comprising:

administering a compound that modulates αvβ₆ integrin, αvβ₁ integrin, or both αvβ₆ integrin and αvβ₁ integrin, wherein the administering is not accompanied by a serious adverse event.

Embodiment C-150. The method of embodiment C-149, wherein the modulating αvβ₆ integrin, αvβ₁ integrin, or both αvβ₆ integrin and αvβ₁ integrin comprises inhibiting αvβ₆ integrin, αvβ₁ integrin, or both αvβ₆ integrin and αvβ₁ integrin.

Embodiment C-151. The method of any one of embodiments C-1 to C-150, wherein the pirfenidone or a pharmaceutically acceptable salt thereof is deuterated pirfenidone or a pharmaceutically acceptable salt thereof.

Embodiment C-152. The method of embodiment C-151, wherein the deuterated pirfenidone is of the formula:

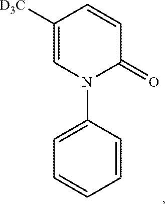

or a pharmaceutically acceptable salt thereof.

Embodiment C-153. A method of increasing the expression of one or more genes in a subject in need thereof, comprising administering (S)-4-((2-methoxyethyl)(4-(5,6,7,8-tetrahydro-1,8-naphthyridin-2-yl)butyl)amino)-2-(quinazolin-4-ylamino)butanoic acid and nintedanib to the subject, wherein said one or more genes are selected from ACACA, AKR1B10, APOB, BCL2L1, C3, C6, CCL2, CXCL8, CYP4A11/22, DAPK1, DLL1, EGFR, ELOVL6, EPHX2, F11R, FASN, FLNB, FZD5, GCNT1, GPC4, HADH, IL1RAP, IL20RB, JAG2, KIR2DL3, KLRB1, LYN, MS4A1, MUC5B, PLIN4, PPARGC1A, PTGER4, SAA1, SCD, SCIN, SLC25A10, SLC2A2, SPIB, SREBF1, or VAMP8.

Embodiment C-154. A method of increasing the expression of one or more genes in a subject in need thereof, comprising administering (S)-4-((2-methoxyethyl)(4-(5,6,7,8-tetrahydro-1,8-naphthyridin-2-yl)butyl)amino)-2-(quinazolin-4-ylamino)butanoic acid and pirfenidone to the subject, wherein said one or more genes are selected from BCL2L1, C3, CCL4, CD209, CYP2J2, EGFR, FLNB, GPC4, GZMA, HCAR2, HDC, IL1B, JAG2, LYN, MAPK10, MMP12, MUC5B, SLC25A10, SPIB, SREBF1, TJP2, TNF, or VAMP8.

Embodiment C-155. A method of decreasing the expression of one or more genes in a subject in need thereof, comprising administering (S)-4-((2-methoxyethyl)(4-(5,6,7,8-tetrahydro-1,8-naphthyridin-2-yl)butyl)amino)-2-(quinazolin-4-ylamino)butanoic acid and nintedanib to the subject, wherein said one or more genes are selected from APOC2, CDH2, COL1A1, COL4A2, FCGR3A/B, ITGB3, LOXL2, NID1, SERPINH1, SPP1, TGFB1, THBS2, FAP, LOX, PDGFRB, POSTN, or SERPINE1.

Embodiment C-156. A method of decreasing the expression of one or more genes in a subject in need thereof, comprising administering (S)-4-((2-methoxyethyl)(4-(5,6,7,8-tetrahydro-1,8-naphthyridin-2-yl)butyl)amino)-2-(quinazolin-4-ylamino)butanoic acid and pirfenidone to the subject, wherein said one or more genes are selected from CDH2, COL1A1, COL5A3, ITGA5, or THBS2.

Embodiment C-157. The method of any one of embodiments C-153 to C-156, wherein the (S)-4-((2-methoxyethyl)(4-(5,6,7,8-tetrahydro-1,8-naphthyridin-2-yl)butyl)amino)-2-(quinazolin-4-ylamino)butanoic acid and nintedanib, or the (S)-4-((2-methoxyethyl)(4-(5,6,7,8-tetrahydro-1,8-naphthyridin-2-yl)butyl)amino)-2-(quinazolin-4-ylamino)butanoic acid and pirfenidone, are administered in an amount effective to have the indicated effect on gene expression.

Embodiment C-158. The method of any one of embodiments C-153 to C-157, wherein the subject has a fibrotic disorder.

Embodiment C-159. The method of embodiment C-158, wherein the fibrotic disorder is idiopathic pulmonary fibrosis.

Embodiment C-160. A method of modulating the activity of at least one gene affecting fibrotic activity in a subject in need thereof, comprising administering (S)-4-((2-methoxyethyl)(4-(5,6,7,8-tetrahydro-1,8-naphthyridin-2-yl)butyl)amino)-2-(quinazolin-4-ylamino)butanoic acid and nintedanib, or administering (S)-4-((2-methoxyethyl)(4-(5,6,7,8-tetrahydro-1,8-naphthyridin-2-yl)butyl)amino)-2-(quinazolin-4-ylamino)butanoic acid and pirfenidone, wherein the at least one gene is substantially modulated by administering (S)-4-((2-methoxyethyl)(4-(5,6,7,8-tetrahydro-1,8-naphthyridin-2-yl)butyl)amino)-2-(quinazolin-4-ylamino)butanoic acid and nintedanib, or by administering (S)-4-((2-methoxyethyl)(4-(5,6,7,8-tetrahydro-1,8-naphthyridin-2-yl)butyl)amino)-2-(quinazolin-4-ylamino)butanoic acid and pirfenidone, but is not substantially modulated by administering only (S)-4-((2-methoxyethyl)(4-(5,6,7,8-tetrahydro-1,8-naphthyridin-2-yl)butyl)amino)-2-(quinazolin-4-ylamino)butanoic acid, administering only nintedanib, or administering only pirfenidone.

Embodiment C-161. The method of Embodiment C-160, wherein the modulating the activity is decreasing the activity.

### SYNTHETIC PROCEDURES

The chemical reactions in the Synthetic Procedures and the Synthetic Examples described can be readily adapted to prepare a number of other compounds of the invention, and alternative methods for preparing the compounds of this invention are deemed to be within the scope of this invention. For example, the synthesis of non-exemplified compounds according to the invention can be successfully performed by modifications apparent to those skilled in the art, e.g., by appropriately protecting interfering groups, by utilizing other suitable reagents known in the art other than those described, or by making routine modifications of reaction conditions. Alternatively, other reactions disclosed herein or known in the art will be recognized as having applicability for preparing other compounds of the invention.

For the examples described herein, reference to a General Procedure indicates that the reaction was prepared using similar reaction conditions and parameters as the General Procedures stated above.

### Procedures

Compounds provided herein may be prepared according to Schemes, as exemplified by the Procedures and Examples. Minor variations in temperatures, concentrations, reaction times, and other parameters can be made when following the Procedures, which do not substantially affect the results of the procedures.

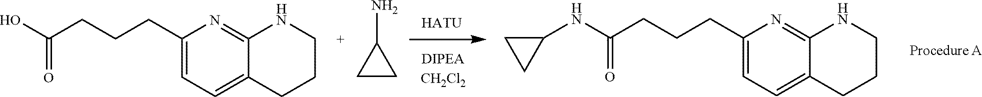

N-cyclopropyl-4-(5,6,7,8-tetrahydro-1,8-naphthyridin-2-yl)butanamide.To a mixture of 4-(5,6,7,8-tetrahydro-1,8-naphthyridin-2-yl)butanoic acid hydrochloride (5.0 g, 19.48 mmol) and cyclopropanamine (1.51 mL, 21.42 mmol) in CH₂Cl₂ (80 mL) at rt was added DIPEA (13.57 mL, 77.9 mmol). To this was then added HATU (8.1 g, 21.42 mmol) and the resulting mixture was stirred at rt for 2 h. The reaction mixture was concentrated in vacuo and purified by normal phase silica gel chromatography to give N-cyclopropyl-4-(5,6,7,8-tetrahydro-1,8-naphthyridin-2-yl)butanamide.

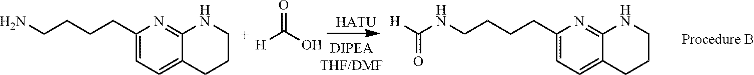

N-(4-(5,6,7,8-tetrahydro-1,8-naphthyridin-2-yl)butyl)formamide. To a mixture of 4-(5,6,7,8-tetrahydro-1,8-naphthyridin-2-yl)butan-1-amine (351 mg, 1.71 mmol) and formic acid (0.09 mL, 2.22 mmol) in 4:1 THF/DMF (5 mL) was added HATU (844 mg, 2.22 mmol) followed by DIPEA (0.89 mL, 5.13 mmol) and the reaction was allowed to stir at rt for 1 h. The reaction mixture was concentrated in vacuo and purified by normal phase silica gel chromatography to give N-(4-(5,6,7,8-tetrahydro-1,8-naphthyridin-2-yl)butyl)formamide.

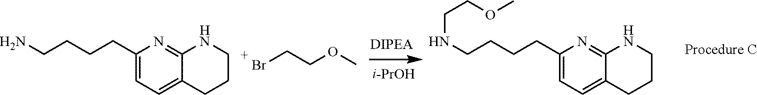

N-(2-methoxyethyl)-4-(5,6,7,8-tetrahydro-1,8-naphthyridin-2-yl)butan-1-amine. A mixture of 4-(5,6,7,8-tetrahydro-1,8-naphthyridin-2-yl)butan-1-amine (300 mg, 1.46 mmol), 1-bromo-2-methoxyethane (0.11 mL, 1.17 mmol) and DIPEA (0.25 mL, 1.46 mmol) in i-PrOH (3 mL) was heated to 70° C. for 18 h. The reaction mixture was allowed to cool to rt and then concentrated in vacuo and purified by normal phase silica gel chromatography to give N-(2-methoxyethyl)-4-(5,6,7,8-tetrahydro-1,8-naphthyridin-2-yl)butan-1-amine.

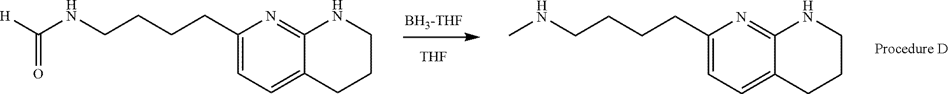

N-methyl-4-(5,6,7,8-tetrahydro-1,8-naphthyridin-2-yl)butan-1-amine. To a solution of N-(4-(5,6,7,8-tetrahydro-1,8-naphthyridin-2-yl)butyl)formamide (200 mg, 0.86 mmol) in THF (2 mL) at rt was added borane tetrahydrofuran complex solution (1.0 M in THF, 4.0 mL, 4.0 mmol) dropwise. The resulting mixture was then heated to 60° C. for 2 h and then allowed to cool to rt. The reaction mixture was diluted with MeOH and concentrated in vacuo. The crude residue was purified by normal phase silica gel chromatography to give N-methyl-4-(5,6,7,8-tetrahydro-1,8-naphthyridin-2-yl)butan-1-amine.

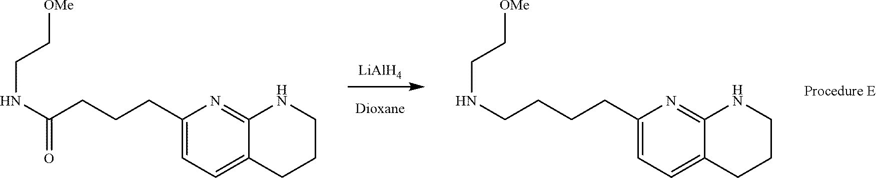

N-(2-methoxyethyl)-4-(5,6,7,8-tetrahydro-1,8-naphthyridin-2-yl)butan-1-amine (5). To a solution of N-(2-methoxyethyl)-4-(5,6,7,8-tetrahydro-1,8-naphthyridin-2-yl)butanamide (15.5 g, 1.0 equiv) in 1,4-dioxane (124 mL) at rt was slowly added LiAlH₄ (1.0 M in THF, 123 mL, 2.2 equiv) and the resulting mixture was heated to reflux for 20 hours and then cooled to 0° C. To this solution was added H₂O (4.7 mL), then 1 M NaOH (4.7 mL) then H₂O (4.7 mL) and warmed to room temperature and stirred for 30 minutes, at which time, solid MgSO₄ was added and stirred for an additional 30 minutes. The resulting mixture was filtered and the filter cake was washed with THF. The filtrate were concentrated in vacuo to give N-(2-methoxyethyl)-4-(5,6,7,8-tetrahydro-1,8-naphthyridin-2-yl)butan-1-amine.

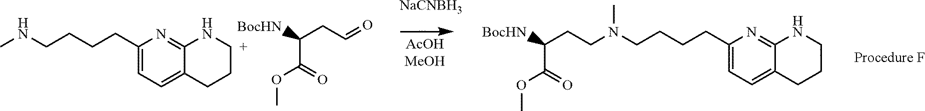

methyl (S)-2-((tert-butoxycarbonyl)amino)-4-(methyl(4-(5,6,7,8-tetrahydro-1,8-naphthyridin-2-yl)butyl)amino)butanoate. To a mixture of N-methyl-4-(5,6,7,8-tetrahydro-1,8-naphthyridin-2-yl)butan-1-amine (5) (187 mg, 0.85 mmol) in MeOH (5 mL) at rt was added acetic acid (0.12 mL, 2.05 mmol) followed by methyl (S)-2-((tert-butoxycarbonyl)amino)-4-oxobutanoate (217 mg, 0.94 mmol). The resulting mixture was allowed to stir at rt for 15 min, at which time, sodium cyanoborohydride (80 mg, 1.28 mmol) was added to the reaction mixture and stirred for 30 min and then concentrated in vacuo. The crude residue was purified by normal phase silica gel chromatography to give methyl (S)-2-((tert-butoxycarbonyl)amino)-4-(methyl(4-(5,6,7,8-tetrahydro-1,8-naphthyridin-2-yl)butyl)amino)butanoate.

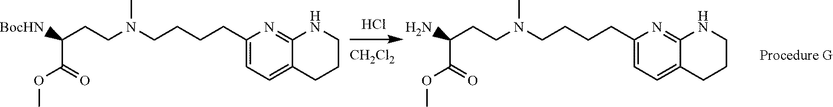

methyl (S)-2-amino-4-(methyl(4-(5,6,7,8-tetrahydro-1,8-naphthyridin-2-yl)butyl)amino)butanoate. To a solution of methyl (S)-2-((tert-butoxycarbonyl)amino)-4-(methyl(4-(5,6,7,8-tetrahydro-1,8-naphthyridin-2-yl)butyl)amino)butanoate (152 mg, 0.35 mmol) in CH₂Cl₂ (2 mL) at rt was added 4 N HCl in 1,4-dioxane (1 mL, 4 mmol) and the resulting mixture was allowed to stir for 2 h. The reaction mixture was concentrated in vacuo to give methyl (S)-2-amino-4-(methyl(4-(5,6,7,8-tetrahydro-1,8-naphthyridin-2-yl)butyl)amino)butanoate as the trihydrochloride salt.

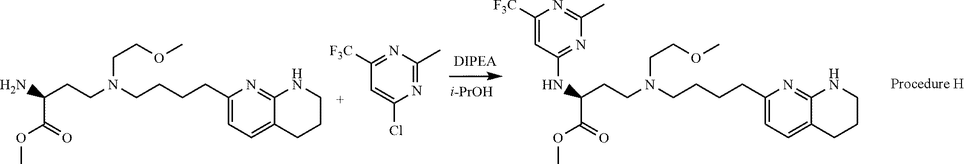

A solution of methyl (S)-2-amino-4-((2-methoxyethyl)(4-(5,6,7,8-tetrahydro-1,8-naphthyridin-2-yl)butyl)amino)butanoate trihydrochloride (80 mg, 0.16 mmol), 4-chloro-2-methyl-6-(trifluoromethyl)pyrimidine (64 mg, 0.33 mmol) and DIPEA (0.23 mL, 1.31 mmol) in i-PrOH (1 mL) was heated at 60° C. overnight. The reaction was allowed to cool to rt and then concentrated in vacuo. The resulting crude residue was purified by normal phase silica gel chromatography to give methyl (S)-4-((2-methoxyethyl)(4-(5,6,7,8-tetrahydro-1,8-naphthyridin-2-yl)butyl)amino)-2-((2-methyl-6-(trifluoromethyl)pyrimidin-4-yl)amino)butanoate.

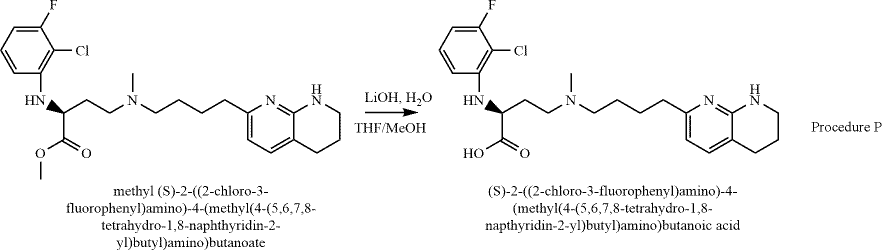

(S)-2-((2-chloro-3-fluorophenyl)amino)-4-(methyl(4-(5,6,7,8-tetrahydro-1,8-naphthyridin-2-yl)butyl)amino)butanoic acid To a solution of methyl (S)-2-((2-chloro-3-fluorophenyl)amino)-4-(methyl(4-(5,6,7,8-tetrahydro-1,8-naphthyridin-2-yl)butyl)amino)butanoate in 4:1:1 THF/MeOH/H₂O at rt was added lithium hydroxide (approximately four equivalents) and the resulting mixture was stirred for 30 min. The reaction mixture was concentrated in vacuo and the resulting crude residue purified by reverse phase HPLC to give (S)-2-((2-chloro-3-fluorophenyl)amino)-4-(methyl(4-(5,6,7,8-tetrahydro-1,8-naphthyridin-2-yl)butyl)amino)butanoic acid.

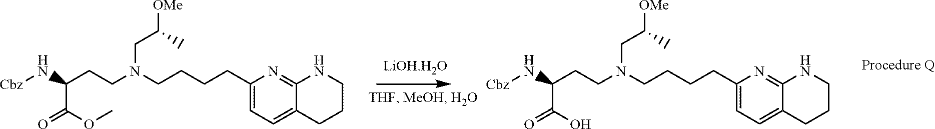

(S)-2-(((benzyloxy)carbonyl)amino)-4-(((R)-2-methoxypropyl)(4-(5,6,7,8-tetrahydro-1,8-naphthyridin-2-yl)butyl)amino)butanoic acid. A mixture of methyl (S)-2-(((benzyloxy)carbonyl)amino)-4-(((R)-2-methoxypropyl)(4-(5,6,7,8-tetrahydro-1,8-naphthyridin-2-yl)butyl)amino)butanoate (1 g, 1.90 mmol) in H₂O (3 mL) and THF (3 mL) and MeOH (3 mL) was added LiOH•H₂O (159.36 mg, 3.80 mmol) and then the mixture was stirred at room temperature for 1 h and the resulting mixture was concentrated in vacuo. The mixture was adjusted to pH=6 by AcOH (2 mL) and the residue was concentrated in vacuo to give a residue to yield compound (S)-2-(((benzyloxy)carbonyl)amino)-4-(((R)-2-methoxypropyl)(4-(5,6,7,8-tetrahydro-1,8-naphthyridin-2-yl)butyl)amino)butanoic acid. LCMS (ESI+): m/z = 513.5 (M+H)⁺. ¹H NMR (400 MHz, DMSO-d): δ ppm 7.25 - 7.37 (m, 5 H) 7.00 (d, J=7.28 Hz, 1 H) 6.81 (br d, J=7.50 Hz, 1 H) 6.22 (d, J=7.28 Hz, 1 H₆) 4.93 - 5.05 (m, 2 H) 3.68 - 3.77 (m, 1 H) 3.25 - 3.34 (m, 1 H) 3.15 - 3.24 (m, 5 H) 2.58 (br t, J=6.06 Hz, 2 H) 2.29 - 2.49 (m, 8 H) 2.16 (br dd, J=12.90, 6.06 Hz, 1 H) 1.69 - 1.78 (m, 2 H) 1.58 - 1.68 (m, 1 H) 1.53 (quin, J=7.39 Hz, 2 H) 1.28 - 1.40 (m, 2 H) 1.00 (d, J=5.95 Hz, 3 H).

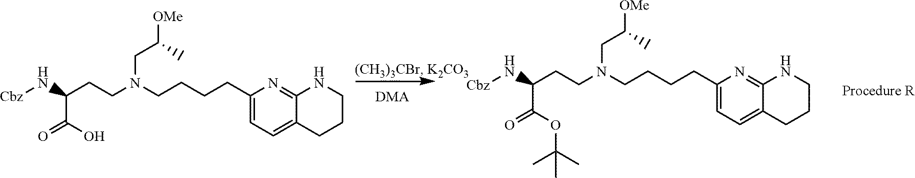

tert-butyl (S)-2-(((benzyloxy)carbonyl)amino)-4-(((R)-2-methoxypropyl)(4-(5,6,7,8-tetrahydro-1,8-naphthyridin-2-yl)butyl)amino)butanoate: A solution of (S)-2-(((benzyloxy)carbonyl)amino)-4-(((R)-2-methoxypropyl)(4-(5,6,7,8-tetrahydro-1,8-naphthyridin-2-yl)butyl)amino)butanoic acid (300 mg, 523.84 µmol, HOAc salt) in DMA (4 mL) was added N-benzyl-N,N-diethylethanaminium chloride (119.32 mg, 523.84 µmol), K₂CO₃ (1.88 g, 13.62 mmol), 2-bromo-2-methylpropane (3.45 g, 25.14 mmol,). The mixture was stirred for 18 h at the 55° C. and then allowed to cool to room temperature. The reaction mixture was concentrated in vacuo and the aqueous phase was extracted with ethyl acetate. The combined organic extracts were washed with brine, dried over Na₂SO₄, filtered, and concentrated in vacuo. The crude residue was purified by prep-TLC to give tert-butyl (S)-2-(((benzyloxy)carbonyl)amino)-4-(((R)-2-methoxypropyl)(4-(5,6,7,8-tetrahydro-1,8-naphthyridin-2-yl)butyl)amino)butanoate. LCMS (ESI+): m/z = 569.3 (M+H)⁺.

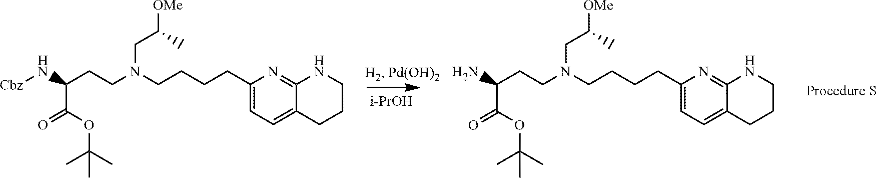

tert-butyl (S)-2-amino-4-(((R)-2-methoxypropyl)(4-(5,6,7,8-tetrahydro-1,8-naphthyridin-2-yl)butyl)amino)butanoate. To a solution of tert-butyl (S)-2-(((benzyloxy)carbonyl)amino)-4-(((R)-2-methoxypropyl)(4-(5,6,7,8-tetrahydro-1,8-naphthyridin-2-yl)butyl)amino)butanoate (107 mg, 188.13 µmol) in i-PrOH (2 mL) was added Pd(OH)₂ (26 mg) under an N₂ atmosphere. The suspension was degassed under vacuum and purged with H₂ several times. The mixture was stirred under H₂ (15 psi) at room temperature for 15 h. The mixture was filtered and concentrated in vacuo to give tert-butyl (S)-2-amino-4-(((R)-2-methoxypropyl)(4-(5,6,7,8-tetrahydro-1,8-naphthyridin-2-yl)butyl)amino)butanoate. LCMS (ESI+): m/z = 435.5 (M+H)⁺. ¹H NMR (400 MHz, CDCl₃): δ ppm 7.06 (d, J=7.34 Hz, 1 H) 6.34 (d, J=7.34 Hz, 1 H) 4.98 (br s, 1 H) 3.38 - 3.44 (m, 4 H) 3.34 (s, 3 H) 2.69 (t, J=6.30 Hz, 2 H) 2.51 - 2.59 (m, 5 H) 2.31 (dd, J=13.39, 5.56 Hz, 1 H) 1.86 - 1.94 (m, 5 H) 1.49 - 1.69 (m, 6 H) 1.47 (s, 9 H) 1.13 (d, J=6.11 Hz, 3 H).

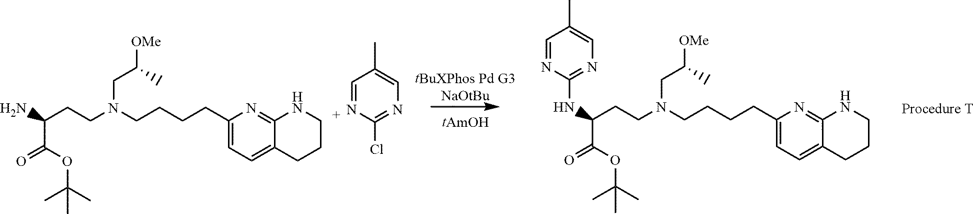

tert-butyl (S)-4-(((R)-2-methoxypropyl)(4-(5,6,7,8-tetrahydro-1,8-naphthyridin-2-yl)butyl)amino)-2-((5-methylpyrimidin-2-yl)amino)butanoate. To a solution of (S)-tert-butyl 2-amino-4-(((R)-2-methoxypropyl)(4-(5,6,7,8-tetrahydro-1,8-naphthyridin-2-yl)butyl)amino)butanoate tert-butyl (S)-2-amino-4-(((R)-2-methoxypropyl)(4-(5,6,7,8-tetrahydro-1,8-naphthyridin-2-yl)butyl)amino)butanoate (100 mg, 230.09 µmol) and 2-chloro-5-methyl-pyrimidine (24.65 mg, 191.74 µmol) in 2-methyl-2-butanol (2 mL) was added t-BuONa (2 M in THF, 191.74 uL) and [2-(2-aminophenyl)phenyl]-methylsulfonyloxy-palladium;ditert-butyl-[2-(2,4,6-triisopropylphenyl)phenyl]phosphane (15.23 mg, 19.17 µmol), and the resulting mixture was stirred at 100° C. for 14 h. The mixture was concentrated in vacuo to give (S)-tert-butyl 4-(((S)-2-methoxypropyl)(4-(5,6,7,8-tetrahydro-1,8-naphthyridin-2-yl)butyl)amino)-2-((5-methylpyrimidin-2-yl)amino)butanoate. LCMS (ESI+): m/z = 527.3 (M+H)⁺.

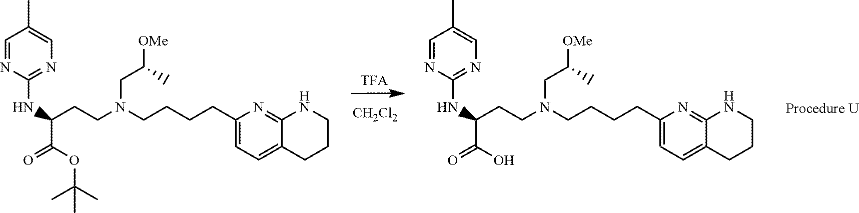

(S)-4-(((R)-2-methoxypropyl)(4-(5,6,7,8-tetrahydro-1,8-naphthyridin-2-yl)butyl)amino)-2-((5-methylpyrimidin-2-yl)amino)butanoic acid. To a solution of tert-butyl (S)-4-(((R)-2-methoxypropyl)(4-(5,6,7,8-tetrahydro-1,8-naphthyridin-2-yl)butyl)amino)-2-((5-methyl pyrimidin-2-yl)amino)butanoate (80 mg, 151.89 µmol) in DCM (2 mL) was added TFA (254.14 mg, 2.23 mmol) at 0° C. The mixture was stirred at room temperature for 6 h. The mixture was concentrated in vacuo and the resulting crude residue was purified by prep-HPLC to give compound (S)-4-(((R)-2-methoxypropyl)(4-(5,6,7,8-tetrahydro-1,8-naphthyridin-2-yl)butyl)amino)-2-((5-methylpyrimidin-2-yl)amino)butanoic acid. LCMS (ESI+): m/z = 471.2 (M+H)⁺. ¹H NMR (400 MHz, Methanol-d₄) δ ppm 8.57 (br s, 2 H) 7.60 (d, J=7.28 Hz, 1 H) 6.67 (d, J=7.28 Hz, 1 H) 4.81 - 4.86 (m, 1 H) 3.86 (br s, 1 H) 3.41 - 3.59 (m, 4 H) 3.39 (s, 3 H) 3.33 - 3.38 (m, 1 H) 3.12 - 3.30 (m, 3 H) 2.76 - 2.86 (m, 4 H) 2.54 (br s, 1 H) 2.39 (br d, J=8.82 Hz, 1 H) 2.30 (s, 3 H) 1.76 - 1.99 (m, 6 H) 1.22 (d, J=5.95 Hz, 3 H).

### SYNTHETIC EXAMPLES

The chemical reactions in the Synthetic Examples described can be readily adapted to prepare a number of other compounds of the invention, and alternative methods for preparing the compounds of this invention are deemed to be within the scope of this invention. For example, the synthesis of non-exemplified compounds according to the invention can be successfully performed by modifications apparent to those skilled in the art, e.g., by appropriately protecting interfering groups, by utilizing other suitable reagents known in the art other than those described, or by making routine modifications of reaction conditions. Alternatively, other reactions disclosed herein or known in the art will be recognized as having applicability for preparing other compounds of the invention.

For the examples described herein, reference to a Procedure indicates that the reaction was prepared using similar reaction conditions and parameters as the Procedures stated above.

### Example A1

Synthesis of (S)-2-fluoro-3-methoxypropan-1-amine

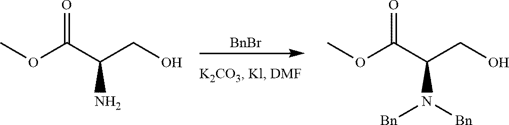

Methyl dibenzyl-D-serinate. To a mixture of methyl D-serinate hydrochloride (100 g, 642.76 mmol) and K₂CO₃ (177.67 g, 1.29 mol) and KI (53.35 g, 321.38 mmol) in DMF (1.5 L) was added benzyl bromide (241.85 g, 1.41 mol) at 0° C. The mixture was stirred at 25° C. for 12 h. The mixture was quenched with H₂O (3000 mL) and EtOAc (1 L × 3). The organic layer was washed with brine (1 L), dried over Na₂SO₄, and concentracted in vacuo. The crude product was purified by normal phase silica gel chromatography to give methyl dibenzyl-D-serinate.

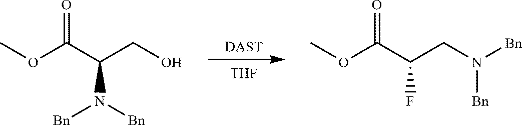

Methyl (S)-3-(dibenzylamino)-2-fluoropropanoate. To a solution of methyl dibenzyl-D-serinate (155 g, 517.77 mmol) in THF (1.2 L) was added DAST (102.65 g, 636.85 mmol, 84.14 mL) dropwise at 0° C. and the reaction mixture was stirred for 14 h at rt. The reaction mixture was quenched with saturated aq. NaHCO₃ (1 L) at 0° C. and extracted with EtOAc (500 mL × 3). The organic phase was dried over Na₂SO₄, filtered, and concentrated in vacuo. The crude product was purified by normal phase silica gel chromatography to give methyl (S)-3-(dibenzylamino)-2-fluoropropanoate.

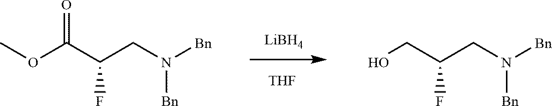 [SMILES: COC(=O)[C@@H](F)CN(Cc1ccccc1)Cc1ccccc1 ; "LiBH4" ; OC[C@@H](F)CN(Cc1ccccc1)Cc1ccccc1 ; "THF"]

(S)-3-(dibenzylamino)-2-fluoropropan-1-ol. To a solution of methyl (S)-3-(dibenzylamino)-2-fluoropropanoate (103 g, 341.79 mmol) in THF (1 L) was added LiBH₄ (14.89 g, 683.58 mmol) at 0° C. The mixture was stirred at 40° C. for 12 h. The mixture was poured into aq. NH₄Cl (500 mL) at 0° C. The aqueous phase was extracted with ethyl acetate (300 mL × 3). The combined organic extracts were dried over Na₂SO₄, filtered, and concentrated in vacuo to give (S)-3-(dibenzylamino)-2-fluoropropan-1-ol that was used without further purification.

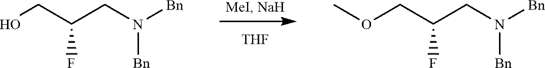

(S)-N,N-dibenzyl-2-fluoro-3-methoxypropan-1-amine. To a solution of (S)-3-(dibenzylamino)-2-fluoropropan-1-ol (51 g, 186.58 mmol) in THF (400 mL) was added NaH (60% dispersion in mineral oil, 11.19 g, 279.87 mmol) at 0° C. and the resulting mixture was stirred at 0° C. for 30 min. To this was then added iodomethane (18.58 mL, 298.52 mmol) and the mixture was stirred at rt for 12 h. The mixture was quenched with aq. NH₄Cl (500 mL) at 0° C. The aqueous phase was extracted with EtOAc (500 mL × 3). The combined organic extracts were dried over Na₂SO₄, filtered, and concentrated in vacuo. The resulting crude residue was purified by normal phase silica gel chromatography to give (S)-N,N-dibenzyl-2-fluoro-3-methoxypropan-1-amine.

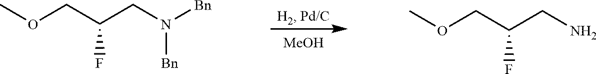

(S)-2-fluoro-3-methoxypropan-1-amine. To a solution of (S)-N,N-dibenzyl-2-fluoro-3-methoxypropan-1-amine (15 g, 52.20 mmol) in MeOH (200 mL) was added Pd/C (3 g). The suspension was degassed under vacuum and purged with H₂ three times. The mixture was stirred under H₂ (50 psi) at 50° C. for 12 h. The reaction mixture was filtered through a pad of Celite and the filtrate was treated with HCl/EtOAc (50 mL) and then concentrated in vacuo to give (S)-2-fluoro-3-methoxypropan-1-amine hydrochloride that was used without further purification.

### Example A2

Synthesis of tert-butyl 7-(4-oxobutyl)-3,4-dihydro-1,8-naphthyridine-1(2H)-carboxylate

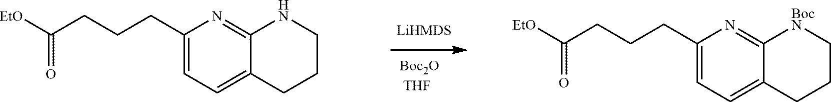

tert-Butyl 7-(4-ethoxy-4-oxobutyl)-3,4-dihydro-1,8-naphthyridine-1(2H)-carboxylate. To a solution of ethyl 4-(5,6,7,8-tetrahydro-1,8-naphthyridin-2-yl)butanoate (5.25 g, 21.1 mmol) and di-tert-butyl dicarbonate (5.89 mL, 25.4 mmol in THF (70 mL) was added lithium bis(trimethylsilyl)amide (25.4 mL, 25.4 mmol) was added at 0° C. After 2 h, the reaction was diluted with EtOAc (50 mL) and was quenched with sat NH₄Cl (50 mL). After 30 min of stirring, the layers were separated and the organic layer was washed with brine (20 mL), dried over Na₂SO₄, and concentrated in vacuo. The resulting crude residue was purified by normal phase silica gel chromatography to give tert-butyl 7-(4-ethoxy-4-oxobutyl)-3,4-dihydro-1,8-naphthyridine-1(2H)-carboxylate.

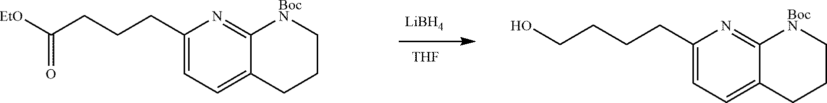 [SMILES: CCOC(=O)CCCc1ccc2c(n1)N(C(=O)OC(C)(C)C)CCC2 ; "LiBH4" ; CC(C)(C)OC(=O)N1CCCc2ccc(CCCCO)nc21 ; "THF"]

tert-Butyl 7-(4-hydroxybutyl)-3,4-dihydro-1,8-naphthyridine-1(2H)-carboxylate. To a solution of tert-butyl 7-(4-ethoxy-4-oxobutyl)-3,4-dihydro-1,8-naphthyridine-1(2H)-carboxylate (6.81 g, 19.5 mmol) in THF (50 mL) was added LiBH₄ (1.0 M in THF, 19.5 mL, 19.5 mmol) at rt. The mixture was stirred overnight and then quenched with sat. NH₄Cl and diluted with EtOAc. The layers were separated and the aqueous layer was extracted with EtOAc. The combined organic extracts were washed with H₂O, dried over Na₂SO₄, filtered, and concentrated in vacuo. The resulting crude residue was purified by normal phase silica gel chromatography to give tert-butyl 7-(4-hydroxybutyl)-3,4-dihydro-1,8-naphthyridine-1(2H)-carboxylate.

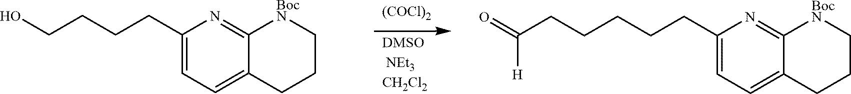

tert-Butyl 7-(4-oxobutyl)-3,4-dihydro-1,8-naphthyridine-1(2H)-carboxylate. A solution of oxalyl chloride (2.57 mL, 29.3 mmol) in CH₂Cl₂ (69 mL) was cooled to -78° C. for 5 minutes, at which time, dimethyl sulfoxide (4.2 mL, 58.6 mmol) was added and the mixture was stirred for 30 min. A solution of tert-butyl 7-(4-hydroxybutyl)-3,4-dihydro-2H-1,8-naphthyridine-1-carboxylate (6.9 g, 22.6 mmol) in CH₂Cl₂ (10.5 mL) was added and stirred at -78° C. for 1 h. Triethylamine (10.5 mL, 75.1 mmol) was then added to the reaction mixture and stirred for 30 mins. The reaction was quenched with water and extracted with CH₂Cl₂. The organic layer was collected and dried over sodium sulfate. The organic layer was concentrate to give tert-butyl 7-(4-oxobutyl)-3,4-dihydro-1,8-naphthyridine-1(2H)-carboxylate that was used without further purification.

### Example A3

Synthesis of methyl (S)-4-((2-methoxyethyl)(4-(5,6,7,8-tetrahydro-1,8-naphthyridin-2-yl)butyl)amino)-2-(quinolin-4-ylamino)butanoate

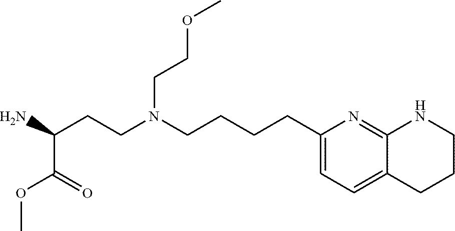 [SMILES: COCCN(CCCCc1ccc2c(n1)NCCC2)CC[C@H](N)C(=O)OC]

Methyl (S)-2-amino-4-((2-methoxyethyl)(4-(5,6,7,8-tetrahydro-1,8-naphthyridin-2-yl) butyl)amino)butanoate. Prepared according to Scheme A using Procedure A with 2-methoxyethylamine, then Procedure E, Procedure F, and Procedure G to give methyl (S)-2-amino-4-((2-methoxyethyl)(4-(5,6,7,8-tetrahydro-1,8-naphthyridin-2-yl)butyl)amino)butanoate.

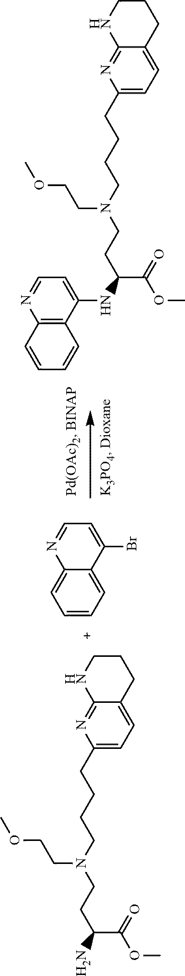

Methyl (S)-4-((2-methoxyethyl)(4-(5,6,7,8-tetrahydro-1,8-naphthyridin-2-yl)butyl) amino)-2-(quinolin-4-ylamino)butanoate. A microwave vial containing methyl (S)-2-amino-4-((2-methoxyethyl)(4-(5,6,7,8-tetrahydro-1,8-naphthyridin-2-yl)butyl)amino)butanoate (125 mg, 0.3 mmol) was charged with 4-bromoquinoline (65 mg, 0.3 mmol), Pd(OAc)₂ (6.3 mg, 0.03 mmol), rac-BINAP (35 mg, 0.6 mmol), and K₃PO₄ (210 mg, 1.0 mmol) and then diluted with Dioxane (2 mL). The mixture was degassed and then sealed and heated to 100° C. for 1 h. The reaction mixture was allowed to cool to rt and then filtered and concentrated in vacuo. The crude residue was purified by normal phase silica gel chromatography to give methyl (S)-4-((2-methoxyethyl)(4-(5,6,7,8-tetrahydro-1,8-naphthyridin-2-yl)butyl)amino)-2-(quinolin-4-ylamino)butanoate.

### Example A4

Synthesis of methyl (S)-2-(isoquinolin-1-ylamino)-4-((2-methoxyethyl)(4-(5,6,7,8-tetrahydro-1,8-naphthyridin-2-yl)butyl)amino)butanoate

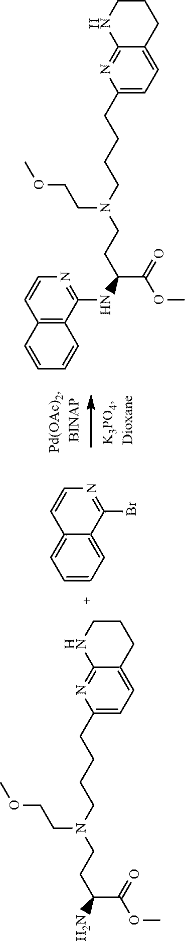

Methyl (S)-2-(isoquinolin-1-ylamino)-4-((2-methoxyethyl)(4-(5,6,7,8-tetrahydro-1,8-naphthyridin-2-yl)butyl)amino)butanoate. A microwave vial containing methyl (S)-2-amino-4-((2-methoxyethyl)(4-(5,6,7,8-tetrahydro-1,8-naphthyridin-2-yl)butyl)amino)butanoate (125 mg, 0.3 mmol) was charged with 1-bromoisoquinoline (65 mg, 0.3 mmol), Pd(OAc)₂ (6.3 mg, 0.03 mmol), rac-BINAP (35 mg, 0.6 mmol), and K₃PO₄ (210 mg, 1.0 mmol) and then diluted with Dioxane (2 mL). The mixture was degassed and then sealed and heated to 100° C. for 1 h. The reaction mixture was allowed to cool to rt and then filtered and concentrated in vacuo. The crude residue was purified by normal phase silica gel chromatography to give methyl (S)-2-(isoquinolin-1-ylamino)-4-((2-methoxyethyl)(4-(5,6,7,8-tetrahydro-1,8-naphthyridin-2-yl)butyl)amino) butanoate.

In the following examples, compounds without specific synthetic descriptions may be synthesized by procedures described herein, for example, analogous to that for compound 2, Scheme 1; compound 81, Scheme 5; and Compound 213, Scheme 24.

For example, (S)-2-((3-cyanopyrazin-2-yl)amino)-4-((2-(3,5-difluorophenoxy)ethyl)(4-(5,6,7,8-tetrahydro-1,8-naphthyridin-2-yl)butyl)amino)butanoic acid (compound 597) may be prepared by slight modification of the procedures from Scheme 1. In step 1, 2-(3,5-difluorophenoxy)ethan-1-amine may be substituted for cyclopropylamine which may afford the analogous amine product. The amine product may then undergo a Boc deprotection as in step 2 followed by a reductive amination as in step 3 to afford an analogous tertiary amine product. This tertiary amine may then undergo a base mediated hydrolysis as in step 4 followed by deprotection of the benzyl carbamate under reductive conditions as in step 5 to afford an analogous amino acid product. This amino acid may then be reacted with a suitably activated heterocycle in an S_{N}Ar reaction, such as 3-chloropyrazine-2-carbonitrile to give the described compound. Similarly, the analogous free amino acid product from step 5 may be reacted with an analogous activated heterocycle as depicted in step 6 and then subjected to either reducing conditions as shown in step 7 of Scheme 1 or cross-coupling conditions as shown in step 2 of Scheme 5 to afford further prophetic compounds described.

The tertiary amine products arising from step 3 in Scheme 1, if alternative amines were substituted for cyclopropylamine, may alternatively be hydrolyzed as depicted in step 1 of Scheme 24 followed by t-butylation of the acid product with t-butyl bromide under basic conditions as shown in step 2 of Scheme 24. The resulting t-butyl ester product may be deprotected under reductive conditions as in step 3 of Scheme 24 to afford an amino ester product, which may then undergo palladium catalyzed cross-coupling with an appropriate aryl or heteroaryl halide as in step 4 of Scheme 24 to give an ester product that may be exposed to acid to generate a final compound as in step 5 of Scheme 24.

For example, (S)-4-((2-(3,5-difluorophenoxy)ethyl)(4-(5,6,7,8-tetrahydro-1,8-naphthyridin-2-yl)butyl)amino)-2-((1-methyl-1H-indazol-3-yl)amino)butanoic acid (compound 624) may be prepared by slight modification of the procedures from Scheme 1. In step 1, 2-(3,5-difluorophenoxy)ethan-1-amine may be substituted for cyclopropylamine which would afford the analogous amine product. This amine product may then undergo a Boc deprotection as in step 2 followed by a reductive amination as in step 3 to afford an analogous tertiary amine product. The tertiary amine product may be hydrolyzed as depicted in step 1 of Scheme 24 followed by t-butylation of the acid product with t-butyl bromide under basic conditions as shown in step 2 of Scheme 24. The resulting t-butyl ester product may be deprotected under reductive conditions as in step 3 of Scheme 24 to afford an amino ester product, which may then undergo palladium catalyzed cross-coupling substituting 3-bromo-1-methyl-1H-indazole for 6-chloro-N,N-dimethylpyrimidin-4-amine in step 4 of Scheme 24 to give an ester product that may be exposed to acid to generate the described compound.

Compound 1: (S)-4-(cyclopropyl(4-(5,6,7,8-tetrahydro-1,8-naphthyridin-2-yl) butyl)amino)-2-((6-(difluoromethyl)pyrimidin-4-yl) amino) butanoic acid. Prepared according to Scheme A using Procedure A with cyclopropylamine, and Procedure H with 4-chloro-6-(difluoromethyl)pyrimidine. LCMS theoretical m/z = 475.3. [M+H]+, found 475.2.

Compound 1: (S)-4-(cyclopropyl(4-(5,6,7,8-tetrahydro-1,8-naphthyridin-2-yl) butyl)amino)-2-((6-(difluoromethyl)pyrimidin-4-yl) amino) butanoic acid. Prepared according to Scheme A using Procedure A with cyclopropylamine, and Procedure H with 4-chloro-6-(difluoromethyl)pyrimidine. LCMS theoretical m/z = 475.3. [M+H]+, found 475.2.

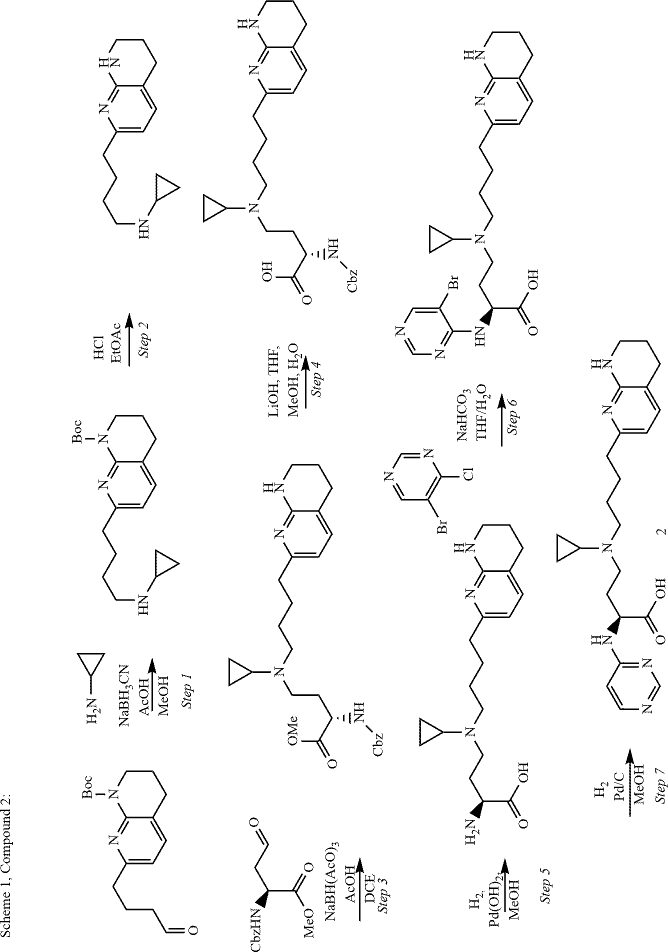

Step 1: tert-butyl 7-(4-(cyclopropylamino) butyl)-3,4-dihydro-1,8-naphthyridine-1(2H)-carboxylate. To a solution of cyclopropanamine (22.8 mL, 328.5 mmol), AcOH (18.8 mL, 328.5 mmol), and NaBH₃CN (4.13 g, 65.7 mmol) in MeOH (100 mL) at 0° C. was added a solution of tert-butyl 7-(4-oxobutyl)-3,4-dihydro-1,8-naphthyridine-1(2H)-carboxylate (10.0 g, 32.9 mmol) in MeOH (100 mL) and the resulting mixture was stirred at rt for 16 h. The mixture was diluted with sat. NaHCO₃ and stirred until gas evolution ceased and then concentrated in vacuo to remove the volatiles. The aqueous layer was extracted with EtOAc and the combined organic extracts were dried over Na₂SO₄, filtered, and concentrated in vacuo. The crude residue was purified by prep-HPLC to give the title compound. LCMS theoretical m/z = 346.3. [M+H]+, found 346.5.

Step 2: N-(4-(5,6,7,8-tetrahydro-1,8-naphthyridin-2-yl) butyl)cyclopropanamine. To a solution of tert-butyl 7-(4-(cyclopropylamino)butyl)-3,4-dihydro-1,8-naphthyridine-1(2H)-carboxylate (2.5 g, 7.24 mmol) in EtOAc (10 mL) was added 4 M HCl in EtOAc (1.8 mL) and the resulting mixture was stirred at rt for 12 h and then concentrated in vacuo. The crude residue was used without further purification. LCMS theoretical m/z = 246.2. [M+H]+, found 246.0.

Step 3: methyl (S)-2-(((benzyloxy)carbonyl)amino)-4-(cyclopropyl(4-(5,6,7,8-tetrahydro-1,8-naphthyridin-2-yl) butyl)amino) butanoate. To a mixture of methyl (S)-2-(((benzyloxy)carbonyl)amino)-4-oxobutanoate (2.59 g, 9.8 mmol) and N-(4-(5,6,7,8-tetrahydro-1,8-naphthyridin-2-yl) butyl)cyclopropanamine hydrochloride (2.5 g, 8.9 mmol) in DCE (40 mL) was added AcOH (761 µL, 13.3 mmol) at 0° C. was added NaBH(OAc)₃ (2.82 g, 13.3 mmol) and the resulting mixture was stirred for 1 h at rt. The mixture was diluted with sat. aq. NaHCO₃ and stirred until gas evolution ceased and then was extracted with CH₂Cl₂. The combined organic extracts were washed with brine and then dried over Na₂SO₄, filtered, and concentrated in vacuo. The crude residue was purified by normal phase silica gel chromatography to give the title compound. LCMS theoretical m/z = 495.3. [M+H]+, found 495.4.

Step 4: (S)-2-(((benzyloxy)carbonyl)amino)-4-(cyclopropyl(4-(5,6,7,8-tetrahydro-1,8-naphthyridin-2-yl) butyl)amino) butanoic acid. To a solution of methyl (S)-2-(((benzyloxy)carbonyl)amino)-4-(cyclopropyl(4-(5,6,7,8-tetrahydro-1,8-naphthyridin-2-yl) butyl)amino) butanoate (4 g, 7.9 mmol) in 1:1:1 THF/MeOH/H₂O (36 mL) was added LiOH•H₂O (664 mg, 15.8 mmol) at 0° C. and the resulting mixture was stirred at rt for 1 h. The mixture was then adjusted to pH = 6 by the careful addition of 1 N HCl and then concentrated in vacuo to give the title compound. LCMS theoretical m/z = 480.3 [M]+, found 480.1.

Step 5: (S)-2-amino-4(cyclopropyl(4-(5,6,7,8-tetrahydro-1,8-naphthyridin-2-yl) butyl)amino) butanoic acid. A flask containing (S)-2-(((benzyloxy)carbonyl)amino)-4-(cyclopropyl(4-(5,6,7,8-tetrahydro-1,8-naphthyridin-2-yl) butyl)amino) butanoic acid (4.5 g, 9.4 mmol) was charged with 20 wt% Pd(OH)₂/C (4.5 g) and then diluted with i-PrOH (300 mL) and stirred under an H₂ atmosphere at 50 psi for 48 h at rt. The reaction mixture was filtered through a pad of CELITE® and rinsed with MeOH and then concentrated in vacuo. The crude residue was purified by reverse phase prep-HPLC to give the title compound. LCMS theoretical m/z = 347.2. [M+H]+, found 347.2.

Step 6: (S)-2-((5-bromopyrimidin-4-yl) amino)-4-(cyclopropyl(4-(5,6,7,8-tetrahydro-1,8-naphthyridin-2-yl) butyl)amino) butanoic acid. To a solution of (S)-2-amino-4-(cyclopropyl(4-(5,6,7,8-tetrahydro-1,8-naphthyridin-2-yl) butyl)amino) butanoic acid trifluoroacetate (150 mg, 0.3 mmol) in 4:1 THF/H₂O (3 mL) was added 5-bromo-4-chloropyrimidine (69 mg, 0.4 mmol) and NaHCO₃ (137 mg, 1.63 mmol) and then was stirred at 70° C. for 2 h and then cooled to rt and concentrated in vacuo. The crude residue was used without further purification.

Step 7: (S)-4-(cyclopropyl(4-(5,6,7,8-tetrahydro-1,8-naphthyridin-2-yl) butyl)amino)-2-(pyrimidin-4-ylamino) butanoic acid. A flask containing (S)-2-((5-bromopyrimidin-4-yl) amino)-4-(cyclopropyl(4-(5,6,7,8-tetrahydro-1,8-naphthyridin-2-yl) butyl)amino) butanoic acid (157 mg, 0.3 mmol) was charged with 20 wt% Pd/C (200 mg) and then diluted with MeOH (20 mL) and the resulting mixture was stirred at rt under an H₂ atmosphere for 4 h and then filtered and concentrated in vacuo. The crude residue was purified by reverse phase prep-HPLC to give the title compound. LCMS (ESI+): m/z = 425.2 (M+H)⁺. ¹H NMR (400 MHz, Methanol-d₄): δ ppm 8.34 (s, 1 H) 7.96 (br s, 1 H) 7.18 (d, J=7.21 Hz, 1 H) 6.52 (br s, 1 H) 6.39 (d, J=7.21 Hz, 1 H) 3.87 - 4.65 (m, 1 H) 3.34 - 3.42 (m, 2 H) 2.76 - 2.96 (m, 2 H) 2.70 (br t, J=6.11 Hz, 4 H) 2.54 (br t, J=7.03 Hz, 2 H) 2.14 - 2.26 (m, 1 H) 1.96 - 2.08 (m, 1 H) 1.87 (q, J=5.87 Hz, 3 H) 1.62 (br d, J=4.40 Hz, 4 H) 0.37 - 0.59 (m, 4 H). LCMS theoretical m/z = 425.3. [M+H]+, found 425.2.

Compound 3: (S)-4-(cyclopropyl(4-(5,6,7,8-tetrahydro-1,8-naphthyridin-2-yl) butyl)amino)-2-((1-methyl-1H-pyrazolo[3,4-d]pyrimidin-4-yl) amino) butanoic acid. To a mixture of (S)-2-amino-4-(cyclopropyl(4-(5,6,7,8-tetrahydro-1,8-naphthyridin-2-yl) butyl)amino) butanoic acid hydrochloride (170 mg, 0.4 mmol) in 4:1 THF/H₂O (2.5 mL) was added 4-chloro-1-methyl-1H-pyrazolo[3,4-d]pyrimidine (75 mg, 0.4 mmol) and NaHCO₃ (112 mg, 1.33 mmol) and the resulting mixture was stirred at 70° C. for 1 h. The reaction mixture was cooled to rt and concentrated in vacuo. The resulting crude residue was purified by reverse phase prep-HPLC to give the title compound as the trifluoroacetate salt. ¹H NMR (400 MHz, D₂O): δ ppm 8.32 - 8.47 (m, 2 H) 7.51 (br d, J=6.60 Hz, 1 H) 6.56 (br s, 1 H) 4.85 (br s, 1 H) 4.03 (br s, 3 H) 3.29 - 3.63 (m, 6 H) 2.38 - 2.91 (m, 7 H) 1.64 - 1.95 (m, 6 H) 0.90 - 1.09 (m, 4 H). LCMS theoretical m/z = 479.3. [M+H]+, found 479.2.

Compound 4: (S)-4-((2-hydroxy-2-methylpropyl) (4-(5,6,7,8-tetrahydro-1,8-naphthyridin-2-yl) butyl)amino)-2-(pyrimidin-4-ylamino) butanoic acid. Prepared according to Scheme A using Procedure A with 1-amino-2-methylpropan-2-ol, Procedure H with 4-chloropyrimidine, and Procedure P. LCMS theoretical m/z = 457.3. [M+H]+, found 457.2.

Compound 5: (S)-4-((2-methoxyethyl) (4-(5,6,7,8-tetrahydro-1,8-naphthyridin-2-yl) butyl)amino)-2-(quinazolin-4-ylamino) butanoic acid. Prepared according to Scheme A using Procedure A with 2-methoxyethan-1-amine, Procedure H with 4-chloroquinazoline, and Procedure P. LCMS theoretical m/z = 493.1. [M+H]+, found 493.1.

Syntheses of Compounds 6 through 780 can be carried out using the procedures described herein. Syntheses of Compounds 6 through 780 are also described in U.S.Pat. No. 10,793,564, U.S. Pat. Application Publication No. 2019/0276449, and International Patent Application No. WO 2019/173653.

### BIOLOGICAL EXAMPLES

### Example B1- Solid Phase Integrin Αvβ₆ Binding Assay

Microplates were coated with recombinant human integrin αvβ₆ (2 µg/mL) in PBS (100 µL/well 25° C., overnight). The coating solution was removed, washed with wash buffer (0.05% Tween 20; 0.5 mM MnCl₂; in 1x TBS). The plate was blocked with 200 µL/well of Block Buffer (1% BSA; 5% sucrose; 0.5 mM MnCl₂; in 1x TBS) at 37° C. for 2 h. Dilutions of testing compounds and recombinant TGFβ₁ LAP (0.67 µg/mL) in binding buffer (0.05% BSA; 2.5% sucrose; 0.5 mM MnCl₂; in 1x TBS) were added. The plate was incubated for 2 hours at 25° C., washed, and incubated for 1 hour with Biotin-Anti-hLAP. Bound antibody was detected by peroxidase-conjugated streptavidin. The IC₅₀ values for testing compounds were calculated by a four-parameter logistic regression.

The IC₅₀ values obtained for αvβ₆ integrin inhibition for a first series of selected exemplary compounds are shown in Table B-1. The IC₅₀ values obtained for αvβ₆ integrin inhibition for a second series of selected exemplary compounds are shown in Table B-2. The compounds tested were compound samples prepared according to procedures described in the Synthetic Examples section, with the stereochemical purity as indicated in the Examples. The IC₅₀ values in Tables B-1 and B-2 are presented in four ranges: below 50 nM; from 50 nM to 250 nM; from above 250 nM to 1000 nM; and above 1000 nM.

TABLE B-1

| Compound No. | αvβ₆ Inhibition IC₅₀ (nM) - range | Compound No. | αvβ₆ Inhibition IC₅₀ (nM) - range |
|---|---|---|---|
| 1 | 250-1000 | 2 | 250-1000 |
| | | 4 | 50-250 |
| 5 | <50 | 6 | 50-250 |
| 7 | <50 | 8 | 50-250 |
| 9 | >1000 | 10 | <50 |
| 11 | <50 | 12 | <50 |
| 13 | 50-250 | 14 | <50 |
| 15 | <50 | 16 | <50 |
| 17 | <50 | 18 | <50 |
| 19 | <50 | 20 | <50 |
| 21 | <50 | 22 | <50 |
| 23 | <50 | 24 | <50 |
| 25 | <50 | 26 | <50 |
| 27 | <50 | 28 | <50 |
| 29 | <50 | 30 | <50 |
| 31 | <50 | 32 | <50 |
| 33 | <50 | 34 | >1000 |
| 35 | <50 | 36 | >1000 |
| 37 | 50-250 | 38 | <50 |
| 39 | <50 | 40 | <50 |
| 41 | <50 | 42 | <50 |
| 43 | <50 | 44 | <50 |
| 45 | <50 | 46 | <50 |
| 47 | <50 | 48 | <50 |
| 49 | <50 | 50 | <50 |
| 51 | <50 | 52 | <50 |
| 53 | <50 | 54 | <50 |
| 55 | <50 | 56 | <50 |
| 57 | <50 | 58 | <50 |
| 59 | <50 | 60 | <50 |
| 61 | <50 | 62 | <50 |
| 63 | <50 | 64 | <50 |
| 65 | <50 | 66 | <50 |

TABLE B-2

| Compound No. | αvβ₆ Inhibition IC₅₀ (nM) - range | Compound No. | αvβ₆ Inhibition IC₅₀ (nM) - range |
|---|---|---|---|
| 67 | <50 | 68 | <50 |
| 69 | <50 | 70 | <50 |
| 71 | <50 | 72 | <50 |
| 73 | <50 | 74 | <50 |
| 75 | <50 | 76 | <50 |
| 77 | <50 | 78 | <50 |
| 79 | <50 | 80 | <50 |
| 81 | <50 | 82 | <50 |
| 83 | <50 | 84 | 250-1000 |
| 85 | 250-1000 | 86 | 50-250 |
| 87 | 250-1000 | 88 | >1000 |
| 89 | <50 | 90 | <50 |
| 91 | <50 | 92 | <50 |
| 93 | <50 | 94 | <50 |
| 95 | >1000 | 96 | >1000 |
| 97 | >1000 | 98 | >1000 |
| 99 | 250-1000 | 100 | <50 |
| 101 | 50-250 | 102 | >1000 |
| 103 | >1000 | 104 | >1000 |
| 105 | <50 | 106 | <50 |
| 107 | 250-1000 | 108 | >1000 |
| 109 | <50 | 110 | <50 |
| 111 | <50 | 112 | 250-1000 |
| | | 114 | <50 |
| 115 | 50-250 | 116 | 50-250 |
| 117 | <50 | 118 | >1000 |
| 119 | >1000 | 120 | >1000 |
| 121 | >1000 | 122 | 250-1000 |
| 123 | <50 | 124 | <50 |
| 125 | 50-250 | 126 | >1000 |
| 127 | 250-1000 | 128 | >1000 |
| 129 | <50 | 130 | <50 |
| 131 | 50-250 | 132 | 50-250 |
| 133 | 50-250 | 134 | 50-250 |
| 135 | 50-250 | 136 | <50 |
| 137 | <50 | 138 | <50 |
| 139 | <50 | 140 | <50 |
| 141 | 50-250 | 142 | >1000 |
| 143 | 50-250 | 144 | 50-250 |
| 145 | <50 | 146 | >1000 |
| 147 | 50-250 | | |

### Example B2—The Disclosed Compounds Potently Inhibit Αvβ₆ in a Solid Phase Assay

A third series of exemplary compounds was selected for testing in the solid phase integrin αvβ₆ binding assay. The compounds tested were compound samples prepared according to procedures described in the Synthetic Examples section, with the stereochemical purity as indicated in the Examples. As in Example B1, microplates were coated with recombinant human integrin αvβ₆ (2 µg/mL) in PBS (100 µL/well 25° C., overnight). The coating solution was removed, washed with wash buffer (0.05% Tween 20; 0.5 mM MnCl₂; in 1x TBS). The plate was blocked with 200 µL/well of Block Buffer (1% BSA; 5% sucrose; 0.5 mM MnCl₂; in 1x TBS) at 37° C. for 2 h. Dilutions of testing compounds and recombinant TGFβ₁ LAP (0.67 µg/mL) in binding buffer (0.05% BSA; 2.5% sucrose; 0.5 mM MnCl₂; in 1x TBS) were added. The plate was incubated for 2 hours at 25° C., washed, and incubated for 1 hour with Biotin-Anti-hLAP. Bound antibody was detected by peroxidase-conjugated streptavidin. The IC₅₀ values for testing compounds were calculated by a four-parameter logistic regression.

### Example B3—The Disclosed Compounds Potently Inhibit Αvβ₁ in a Solid Phase Assay

A fourth series of exemplary compounds was selected for testing in a solid phase integrin αvβ₁ binding assay. The compounds tested were compound samples prepared according to procedures described in the Synthetic Examples section, with the stereochemical purity as indicated in the Examples. Similar to Examples B1 and B2, microplates were coated with recombinant human integrin αvβ₁ (2 µg/mL) in PBS (100 µL/well 25° C., overnight). The coating solution was removed, washed with wash buffer (0.05% Tween 20; 0.5 mM MnCl₂; in 1x TBS). The plate was blocked with 200 µL/well of Block Buffer (1% BSA; 5% sucrose; 0.5 mM MnCl₂; in 1x TBS) at 37° C. for 2 h. Dilutions of testing compounds and recombinant TGFβ₁ LAP (0.67 µg/mL) in binding buffer (0.05% BSA; 2.5% sucrose; 0.5 mM MnCl₂; in 1x TBS) were added. The plate was incubated for 2 hours at 25° C., washed, and incubated for 1 hour with Biotin-Anti-hLAP. Bound antibody was detected by peroxidase-conjugated streptavidin. The IC₅₀ values for testing compounds were calculated by a four-parameter logistic regression.

### Example B4—The Disclosed Compounds Potently Inhibit Human Αvβ₆ Integrin

A fifth series of exemplary compounds was selected for determining biochemical potency using the ALPHASCREEN® (Perkin Elmer, Waltham, MA) proximity-based assay (a bead-based, non-radioactive Amplified Luminescent Proximity Homogeneous Assay) as described previously (Ullman EF et al., Luminescent oxygen channeling immunoassay: Measurement of particle binding kinetics by chemiluminescence. Proc. Natl. Acad. Sci. USA, Vol. 91, pp. 5426-5430, June 1994). To gauge the potency of inhibitors of binding to human integrin αvβ₆, inhibitor compounds and integrin were incubated together with recombinant TGFβ₁ LAP and biotinylated anti-LAP antibody plus acceptor and donor beads, following the manufacturer’s recommendations. The donor beads were coated with streptavidin. The acceptor beads had a nitrilotriacetic acid Ni chelator, for binding to a 6xHis-tag on human integrin αvβ₆. All incubations occurred at room temperatures in 50 mM Tris-HCl, pH 7.5, 0.1% BSA supplemented with 1 mM each CaCl₂ and MgCl₂. The order of reagent addition was as follows: 1. αvβ₆ integrin, test inhibitor compound, LAP, biotinylated anti-LAP antibody and acceptor beads were all added together. 2. After 2 hours, donor beads were added. After another 30 min incubation, samples were read.

Integrin binding was evaluated by exciting donor beads at 680 nm, and measuring the fluorescent signal produced, between 520-620 nm, using a Biotek Instruments (Winooski, VT, USA) SynergyNeo2 multimode plate reader. Compound potency was assessed by determining inhibitor concentrations required to reduce fluorescent light output by 50%. Data analysis for IC₅₀ determinations was carried out by nonlinear four parameter logistic regression analysis using Dotmatics ELN Software (Core Informatics Inc., Branford, Ct).

### Example B5—The Disclosed Compounds Potently Inhibit Human Αvβ₁ Integrin

A sixth series of exemplary compounds was selected for determining biochemical potency using the ALPHASCREEN® proximity-based assay as described in Example B4. To gauge the potency of inhibitors of binding to human integrin αvβ₁, inhibitor compounds and integrin were incubated together with biotinylated, purified human fibronectin plus acceptor and donor beads, following the manufacturer’s recommendations. The donor beads were coated with streptavidin. The acceptor beads had a nitrilotriacetic acid Ni chelator, for binding to a 6xHis-tag on human integrin αvβ₁. All incubations occurred at room temperatures in 50 mM Tris-HCl, pH 7.5, 0.1% BSA supplemented with 1 mM each CaCl₂ and MgCh. The order of reagent addition was as follows: 1. αvβ₁ integrin, test inhibitor compound, fibronectin-biotinylated and acceptor beads were all added together. 2. After 2 hours, donor beads were added. After another 30 min incubation, samples were read.

Integrin binding was evaluated by exciting donor beads at 680 nm, and measuring the fluorescent signal produced, between 520-620 nm, using a Biotek Instruments (Winooski, VT, USA) SynergyNeo2 multimode plate reader. Compound potency was assessed by determining inhibitor concentrations required to reduce fluorescent light output by 50%. Data analysis for IC₅₀ determinations was carried out by nonlinear four parameter logistic regression analysis using Dotmatics ELN Software (Core Informatics Inc., Branford, Ct).

### Combined Inhibition Results of Examples B1, B2, B3, B4, and B5

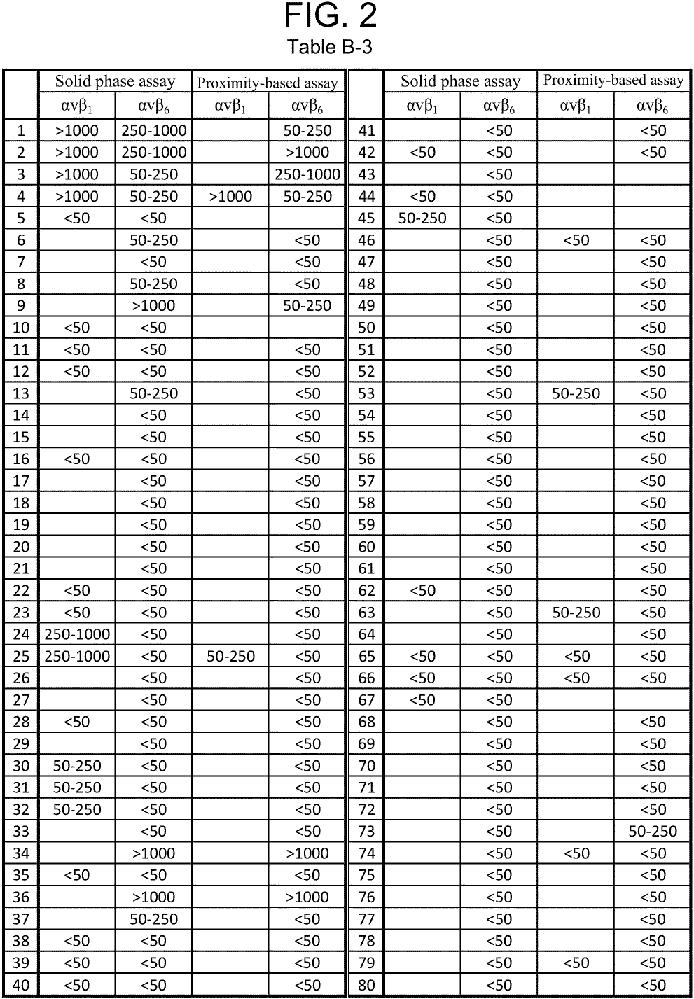

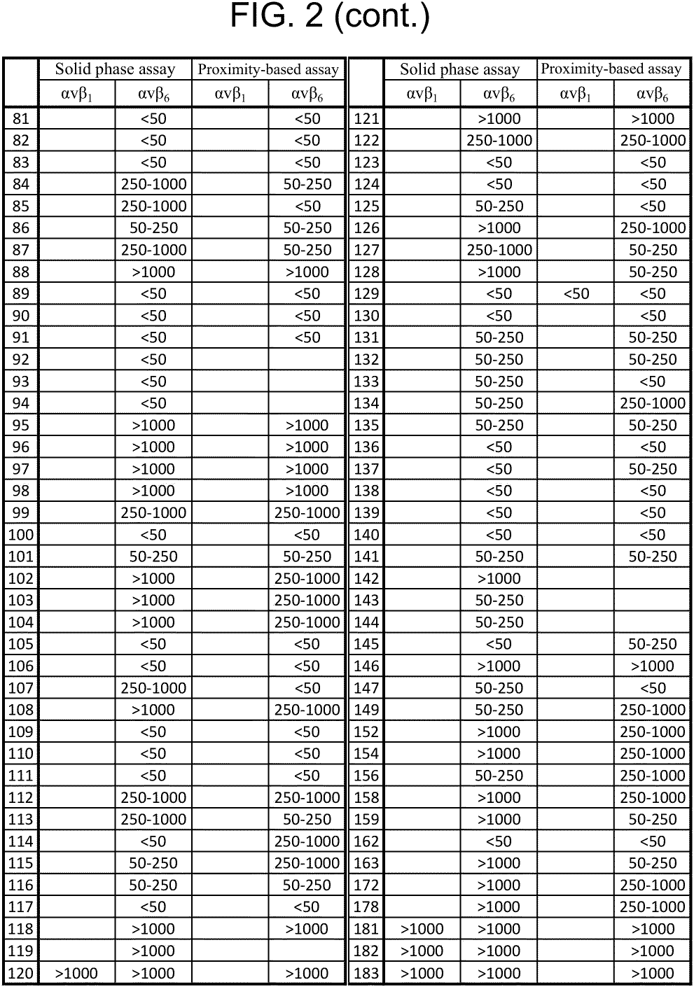

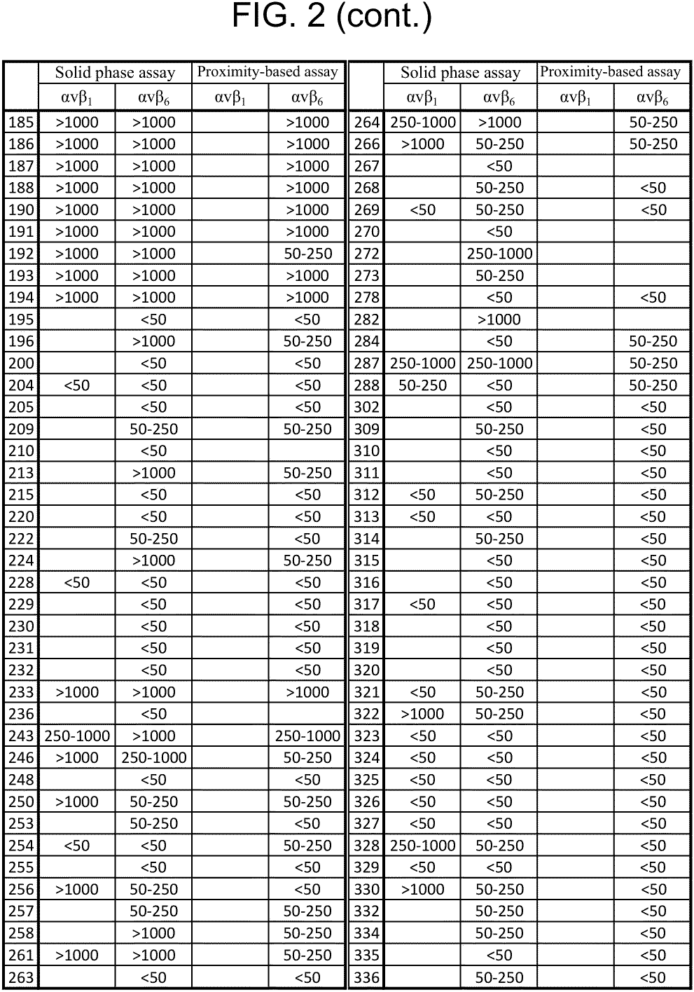

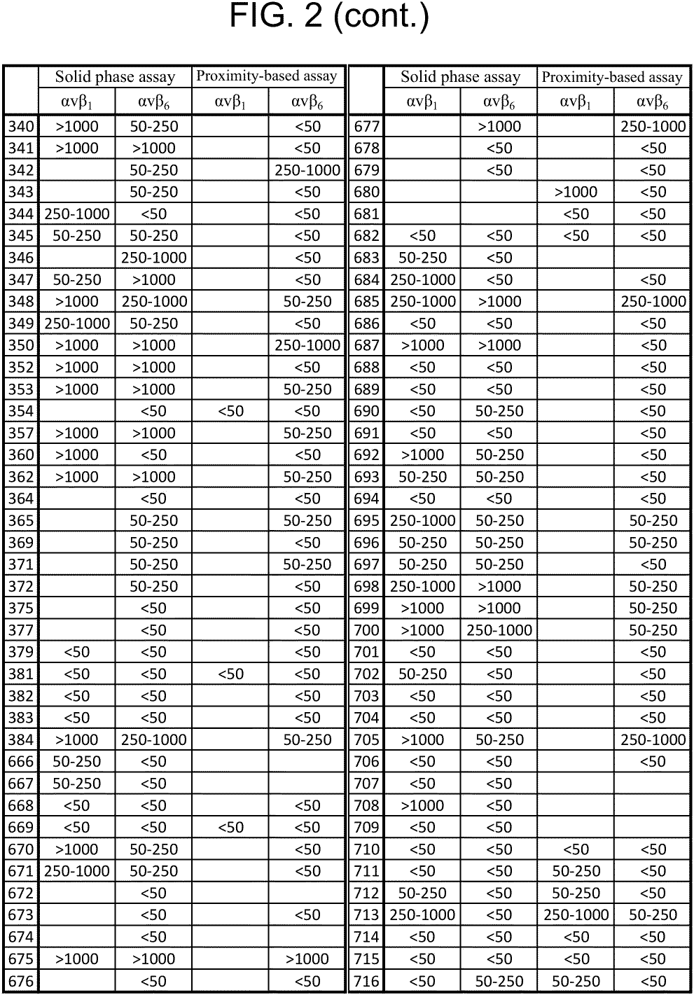

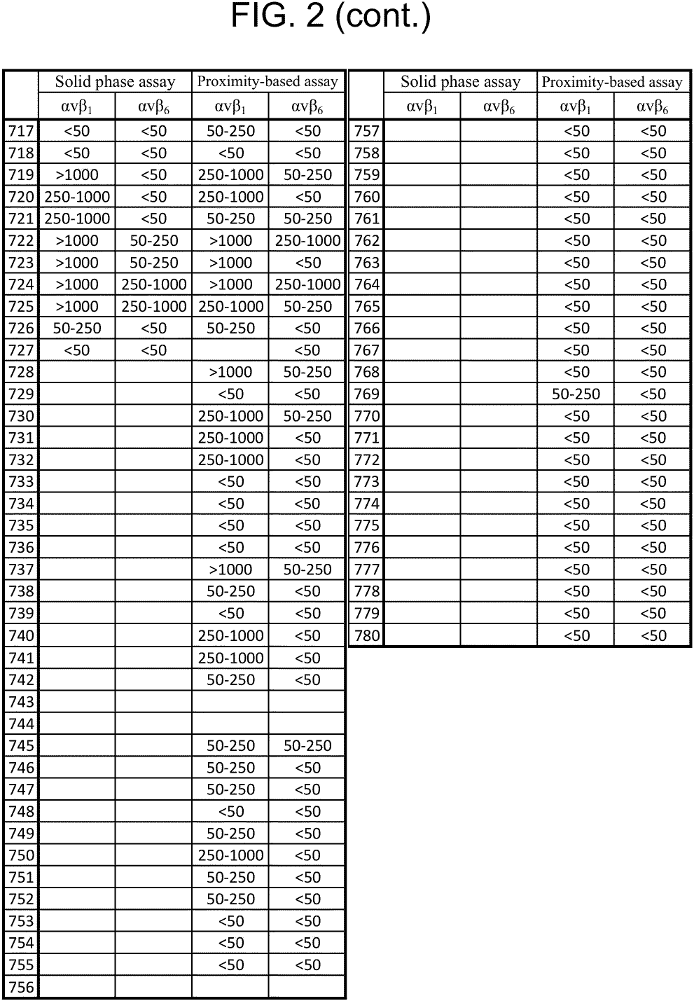

Table B-3 (FIG. 2) shows IC₅₀ data from Examples B1, B2, B3, B4, and B5 for inhibition of αvβ₁ and αvβ₆ integrin in the solid phase assays and inhibition of human αvβ₁ and αvβ₆ integrin in the ALPHASCREEN® assays. The IC₅₀ data is shown in four ranges: below 50 nM; from 50 nM to 250 nM; from above 250 nM to 1000 nM; and above 1000 nM.

### Example B6—αvβ₆ and αvβ₁ Inhibition Activity Shown in Normal Human Bronchial Epithelial Cells and IPF-Derived Human Lung Fibroblasts

Two latency associated peptide (LAP) adhesion binding assays were devised using primary human lung cells, including normal (healthy) human bronchial epithelial cells and human lung fibroblasts (healthy and IPF).

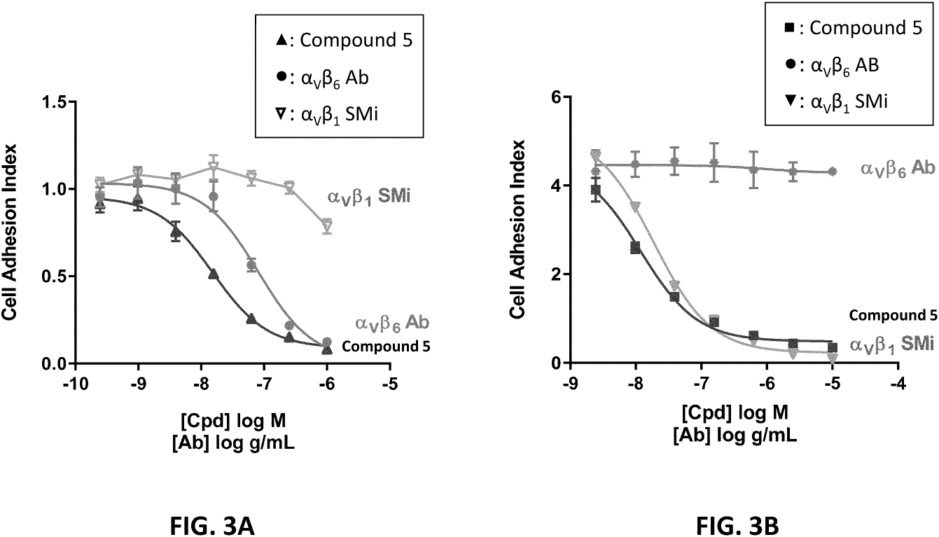

Human bronchial epithelial cells are known to express αvβ₆ integrin in culture. Human bronchial epithelial cells were prepared for the assay by dissociation with trypsin/EDTA and were then seeded at 20,000 cells per well on 96 well plates (ACEA Bioscience E-plate View, Acea Biosciences; San Diego, CA) previously coated with 5 (µg/ml of recombinant human LAP (R&D Systems; Minneapolis, MN) and blocked with 4% bovine serum albumin. Cell index (electrical impedance) was measured to assess cell attachment/spreading every 3 minutes for 24 hours at 37° C. / 5% CO₂ using the xCELLigence RTCA MP Instrument (Acea Biosciences; San Diego, CA). EC₉₀ (time point at 90% of the peak cell index) was determined for vehicle-treated cells and IC₅₀ curves for test article-treated cells were generated at that time point. In the assay, the IPF-derived human bronchial epithelial cells were separately incubated with: a αvβ₁-selective small molecule inhibitor (characterized by sub-50 nM IC₅₀ for αvβ₁, and selective for αvβ₁ over αvβ₆ by a factor of about 25); a selective antibody αvβ₆ inhibitor, 3G9 (ITGB1BP2 Monoclonal Antibody (3G9), ThermoFisher Scientific, Santa Clara, CA); and compound 5, (S)-4-((2-methoxyethyl)(4-(5,6,7,8-tetrahydro-1,8-naphthyridin-2-yl)butyl) amino)-2-(quinazolin-4-ylamino)butanoic acid. FIG. 3A shows that compound 5 and the selective antibody αvβ₆ inhibitor 3G9 both substantially inhibited normal bronchial epithelial cell adhesion to LAP, in contrast with the αvβ₁-selective small molecule inhibitor.

Human lung fibroblasts derived from normal and IPF lung tissue are known to express αvβ₁ integrin. The IPF-derived human lung fibroblasts were prepared for the assay by dissociation with trypsin/EDTA, and were seeded at 20,000 cells per well on 96 well plates (ACEA Bioscience E-plate View, Acea Biosciences; San Diego, CA) previously coated with 5 (µg/ml of recombinant human LAP (R&D Systems; Minneapolis, MN) and blocked with 4% bovine serum albumin. Cell index (electrical impedance) was measured to assess cell attachment/spreading every 3 minutes for 24 hours at 37° C. / 5% CO₂ using the xCELLigence RTCA MP Instrument (Acea Biosciences; San Diego, CA). EC₉₀ (time point at 90% of the peak cell index) was determined for vehicle-treated cells and IC₅₀ curves for test article-treated cells were generated at that time point. In the assay, the IPF-derived human lung fibroblasts were separately incubated with: the αvβ₁-selective small molecule inhibitor; the selective antibody αvβ₆ inhibitor, 3G9; and compound 5. FIG. 3B shows that compound 5 and the αvβ₁-selective small molecule inhibitor both substantially inhibited cell adhesion in the IPF-derived lung fibroblasts, in contrast to the selective antibody αvβ₆ inhibitor, 3G9.

### Example B7—Dual αvβ₆/αvβ₁ Inhibition Reduces Collagen Deposition in the Murine Bleomycin Model

It has been previously shown that inhibition of αvβ₆ in the lung can be detected though measurement of phospho-SMAD (pSMAD) in alveolar macrophages. Alveolar macrophages are known to operate in a unique niche in the lung, distinct from interstitial macrophages. SMAD3 is a downstream target of the active TGF-β cytokine binding its receptor and in alveolar macrophages it is phosphorylated by homoeostatic levels of TGF-β. Accordingly, it was desirable to know whether inhibition of TGF-β activation using the disclosed compounds would result in reduced SMAD2 and SMAD3 phosphorylation.

Mice (C57BL/6) were divided into healthy (n=15), vehicle-treated (n=15), and test article-treated (n=15 per dose) groups. Mice in the vehicle and test article-treated groups were administered 3 U/kg of bleomycin (Teva Pharmaceuticals; North Wales, PA) via oropharyngeal aspiration while under anesthesia on day 0. Healthy animals were administered water in a similar fashion. Starting on day 7, mice in the control group were administered PBS vehicle, 130 uL, by oral gavage, BID for 14 days. Also starting on day 7, mice in the test group were administered compound 5 in PBS by oral gavage, BID for 14 days, at relative dosages of 1x, 2.5x and 5x. The absolute amount of the 1x dosage was selected at a value in mg/kg that showed significant efficacy. From day 14 through day 21, 9 of the 15 mice were administered ²H₂O for labeling. All mice were sacrificed on day 21 and tissues were collected. Samples were prepared for analysis either directly from lung tissue, or by bronchoalveolar lavage, which washes out the bronchiolar and alveolar space with saline to produce a bronchoalveolar lavage fluid (BALF) in which 80-90% of cells are alveolar macrophages.

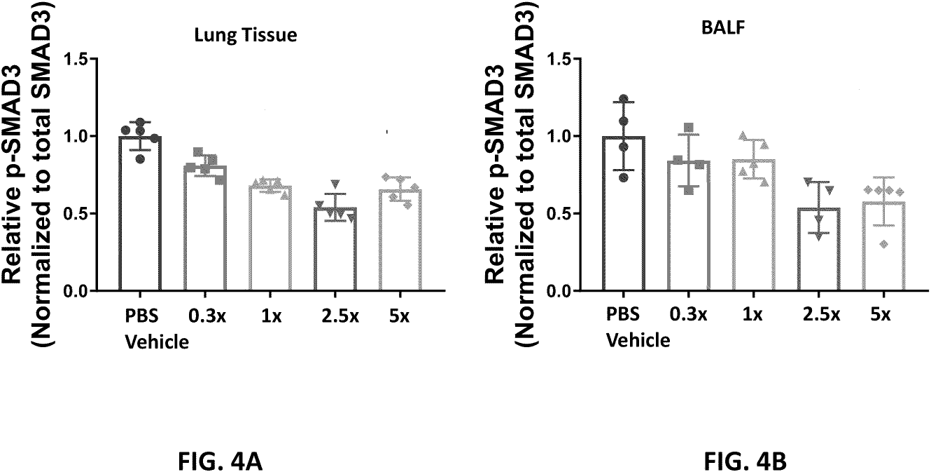

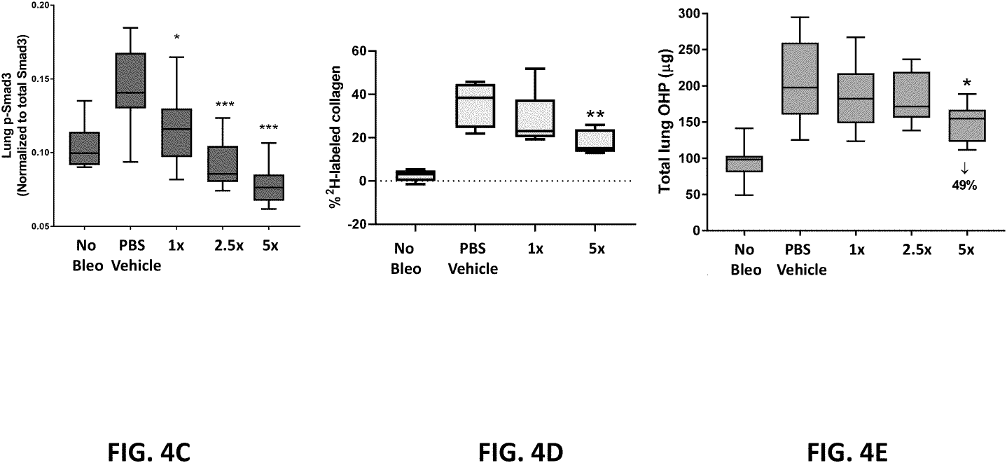

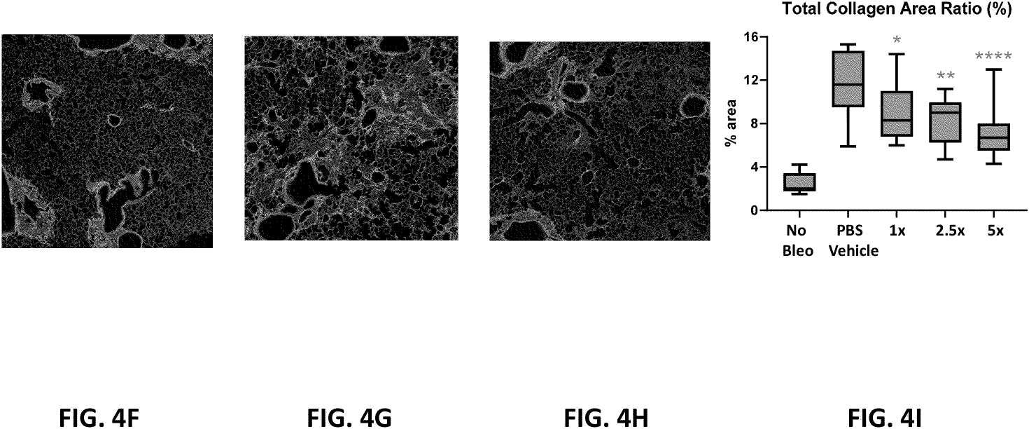

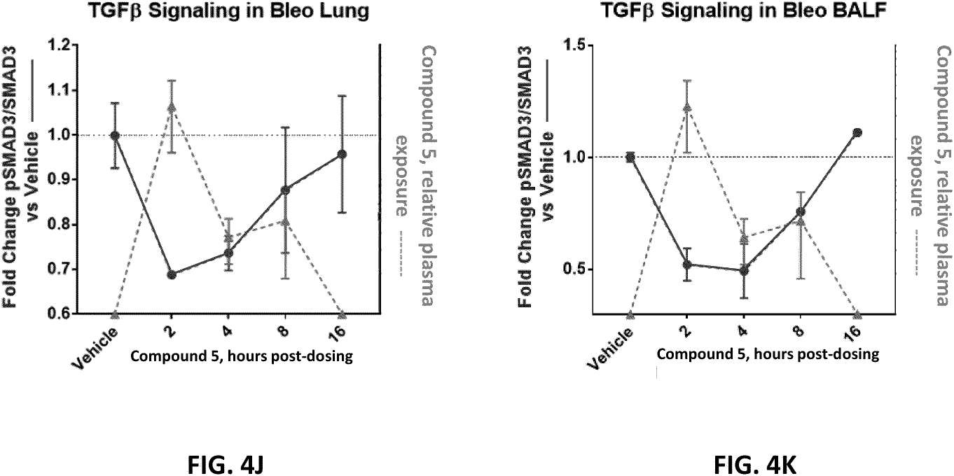

FIG. 4A is a graph of PSMAD3/SMAD3 in lung tissue from healthy mice administered PBS vehicle and varying levels of compound 5 for 4 days. FIG. 4B is a graph of PSMAD3/SMAD3 in BALF drawn from the same healthy mice administered PBS vehicle and varying levels of compound 5 for 4 days. FIGS. 4A and 4B show that 4 days of compound 5 treatment significantly reduced SMAD3 phosphorylation in both lung tissue and cells isolated from BALF in a dose dependent manner to approximately 50% of the untreated levels

FIG. 4C is a graph showing that compared to the healthy mice, lung tissue in the vehicle-treated mice experienced a substantial increase in SMAD3 phosphorylation, which is a measure of TGF-β signaling-related kinase activity. FIG. 4C also shows, compared to the vehicle-treated mice, substantial, statistically significant dose-dependent reductions in SMAD3 phosphorylation in the test article-treated mice according to the dosage of compound 5, including at 1x (p<0.05 vs vehicle), 2.5x (p < 0.01 vs vehicle), and 5x mg/kg (p < 0.001 vs vehicle). This time- and dose-dependent inhibition of pSMAD3 levels in the lung to approximately 50% of the untreated levels was associated with inhibition of fibrosis according to the following results. FIG. 4D is a graph showing that compared to the healthy mice, lung tissue in the vehicle-treated mice experienced a substantial accumulation of new collagen as evidenced by the percentage of lung collagen containing ²H-labeled hydroxyproline. FIG. 4D also shows, compared to the control mice, a dose-dependent reduction in accumulated new collagen as evidenced by the percentage of lung collagen containing ²H-labeled hydroxyproline in the test mice, including at 1x, and at 5x (p < 0.01 vs vehicle). FIG. 4E shows that compared to the healthy mice, the vehicle-treated mice experienced a significant increase in total pulmonary collagen, as measured by µg of hydroxyproline. FIG. 4E also shows, compared to the control mice, a reduction in total pulmonary collagen in the test mice according to the dosage of compound 5, including at 1x, 2.5x, and 5x (p < 0.05 vs vehicle). As shown among FIGS. 4C, 4D, and 4E, in fibrotic bleomycin-treated mice, compound 5 abrogated the increase in pSMAD3 due to bleomycin challenge, a reduction that was associated with inhibition of fibrosis.

FIGS. 4F, 4G, and 4H show high resolution second harmonic generation images of fibrillar collagen (collagen type I and III) taken from formalin-fixed paraffin embedded lung tissue sections from a healthy mouse lung (4F), a vehicle-treated mouse lung (4G) and a test-article treated mouse lung (4H; 500 mg/kg BID). Color scale is indicative of collagen fiber density (red = most dense; blue = least dense).

FIG. 4I is a graph showing the percent total collagen area in the second harmonic generation mouse lung images. Large structural areas of collagen found similarly in healthy and fibrotic tissues (dense collagen fibers surrounding airways were excluded from this analysis to focus on interstitial fibrotic collagen). FIG. 4I shows that compared to the healthy mice, lung tissue in the vehicle-treated mice experienced a substantial increase in total collagen area in the second harmonic generation images. FIG. 4I also shows that compared to the control mice, lung tissue in the test article-treated mice experienced a substantial, statistically significant dose-dependent reduction in total collagen area in the second harmonic generation images according to the administration of compound 5, including at 1x (p<0.05 vs vehicle), 2.5x (p < 0.01 vs vehicle), and 5x (p < 0.0001 vs vehicle). The 1x, 2.5x, and 5x dosages were at the same absolute values in mg/kg as in Example B7.

FIGS. 4J and 4K are graphs of sequential measurements in bleomycin-treated mice, which demonstrated a close inverse relationship between pSMAD3 levels in lung (4J) and BALF cells (4K) vs. plasma drug exposure. The data for FIGS. 4J and 4K, was obtained 14 days after bleomycin challenge in mice were treated with compound 5 at 2.5x dose (PO, BID for 1.5 days).

### Example B8—Dual αvβ₁/αvβ₆ Inhibition Outperforms Single Integrin Inhibition in Precision Cut Lung Slice Assays of Mice Under Acute Bleomycin Exposure

Mice (C57BL/6) were administered 3 U/kg of bleomycin (Teva Pharmaceuticals; North Wales, PA) on day 0 via oropharyngeal aspiration while under anesthesia. On day 14, precision cut lung slices were obtained. Following euthanization, 2% low gelling temp agarose was injected into the mouse lungs via the trachea. Lungs were excised and the inferior lobe separated by dissection. The lobes were then subjected to precision slicing to obtain samples for culture using a microtome (Compresstome VF-300-0Z, Precisionary; Greenville, NC). Individual slices were distributed in a multiwell culture plate and cultured for 3 days under control (DMSO) and test compound conditions. The viability of the slices over the course of culturing was confirmed by WST-1 assay of mitochondrial activity.

During the culture period, slices in the control group were treated with DMSO and slices in the test group were treated with a DMSO solution of one of: a selective antibody αvβ₆ inhibitor, 3G9; the αvβ₁-selective small molecule inhibitor; compound 5; a first pan-αv small molecule inhibitor ((3S)-3-[3-(3,5-dimethyl-1H-pyrazol-1-yl)phenyl]-4-{(3S)-3-[2-(5,6,7,8-tetrahydro-1,8-naphthyridin-2-yl)ethyl]-1-pyrrolidinyl}butanoic acid, PROBECHEM®, St. Petersburg, FL); a second pan-αv small molecule inhibitor ((3S)-N-[3-hydroxy-5-[(1,4,5,6-tetrahydro-5-hydroxy-2-pyrimidinyl)amino]benzoyl]glycyl-3-[3-bromo-5-(1,1-dimethylethyl)phenyl]-(3-alanine, Cayman Chemical, Ann Arbor, MI); and a small molecule ALK5 (TGF-β type I receptor) inhibitor (4-[2-Fluoro-5-[3-(6-methyl-2-pyridinyl)-1H-pyrazol-4-yl]phenyl]-1H-pyrazole-1-ethanol, Bio-Techne Corporation, Minneapolis, MN). Single and dual integrin inhibitors were analyzed at their respective IC₅₀ concentrations for inhibition of TGF-beta activation (compound 5 run at IC₅₀ for (αᵥβ₆). The pan αv integrin inhibitors and small molecule ALK5 inhibitor were analyzed at concentrations 10x above their respective reported IC₅₀ values.

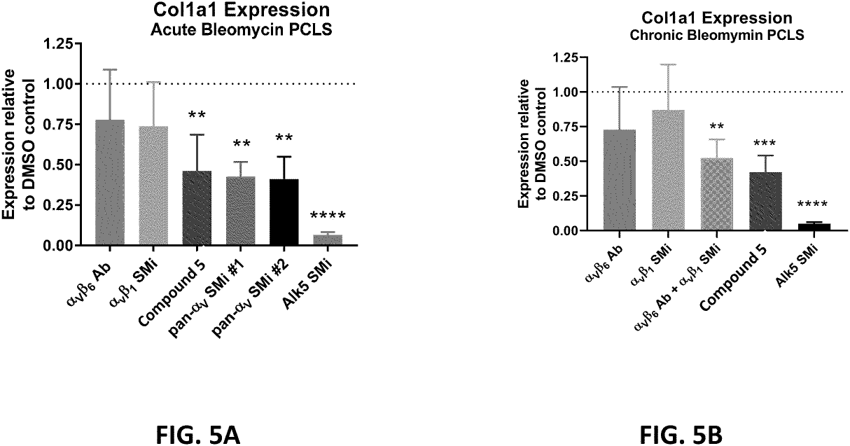

FIG. 5A is a bar graph, normalized to control slices treated with DMSO, showing that all test treatments reduced Type I Collagen gene Col1a1 expression, although selective antibody αvβ₆ inhibitor 3G9 and the αvβ₁-selective small molecule inhibitor were not statistically significant. Compound 5, as a dual αvβ₁/αvβ₆ inhibitor, decreased Type I Collagen gene Col1a1 expression substantially (p<0.01 vs vehicle) compared to the DMSO control, the selective antibody αvβ₆ inhibitor, 3G9; and the αvβ₁-selective small molecule inhibitor. Compound 5 decreased Type I Collagen gene Col1a1 expression comparably to the first and second pan-αv small molecule inhibitors (each p<0.01 compared to the DMSO control). The small molecule ALK5 inhibitor, used as a positive control representative of total TGF-beta signaling inhibition, provided the greatest decrease in Type I Collagen gene Col1a1 expression (p<0.0001 compared to the DMSO control).

### Example B9—Dual αvβ₁/αvβ₆ Inhibition Outperforms Single Integrin Inhibition in Precision Cut Lung Slice Assays of Mice Under Chronic Bleomycin Exposure

Mice (C57BL/6) were administered 3 U/kg of bleomycin (Teva Pharmaceuticals; North Wales, PA) on Day 0 and 1 U/kg of bleomycin on days 14, 28, 42 and 56 via oropharyngeal aspiration while under anesthesia. At day 70, 14 days after the final bleomycin insult, precision cut lung slices were obtained. Following euthanization, 2% low gelling temp agarose was injected into the mouse lungs via the trachea. Lungs were excised and the inferior lobe separated by dissection. The lobes were then subjected to precision slicing to obtain samples for culture using a microtome (Compresstome VF-300-0Z, Precisionary; Greenville, NC). Individual slices were distributed in a multiwell culture plate and cultured for 7 days under control (DMSO) and test compound conditions. The viability of the slices over the course of culturing was confirmed by WST-1 assay of mitochondrial activity.

During the culture period, slices in the control group were treated with DMSO and slices in the test group were treated with a DMSO solution of one of: the selective antibody αvβ₆ inhibitor 3G9; the αvβ₁-selective small molecule inhibitor; a combination of the selective antibody αvβ₆ inhibitor 3G9 and the αvβ₁-selective small molecule inhibitor; compound 5; and the small molecule ALK5 inhibitor. The selective αvβ₁ and αvβ₆ integrin inhibitors were analyzed at ≥ their respective IC₉₀ concentrations for inhibition of TGF-beta activation. Compound 5 was run at approximate IC₅₀ for inhibition of TGF-beta activation by αvβ₆. The small molecule ALK5 inhibitor was analyzed at 10x its reported IC₅₀ value.

FIG. 5B is a bar graph, normalized to control slices treated with DMSO, showing that all test treatments reduced lung Col1a1 expression. Compound 5, as a dual αvβ₁/αvβ₆ inhibitor, decreased lung Col1a1 expression substantially (p<0.01 vs vehicle) compared to the DMSO control, the selective antibody αvβ₆ inhibitor, 3G9; and the αvβ₁-selective small molecule inhibitor. Compound 5 also decreased lung Col1a1 expression to a greater extent than combined administration (p<0.001 vs vehicle) of the selective antibody αvβ₆ inhibitor 3G9 and the αvβ₁-selective small molecule αvβ₁ inhibitor. The small molecule ALK5 inhibitor, used as a positive control representative of total TGF-beta signaling inhibition, provided the greatest decrease in Type I Collagen gene Col1a1 expression (p<0.0001 compared to the DMSO control).

### Example B10—Dual αvβ₁/αvβ₆ Inhibition More Potently Blocks Collagen Gene Expression in the Murine Bleomycin Model than Pirfenidone and Nintedanib

Mice (C57BL/6) were administered 3 U/kg of bleomycin (Teva Pharmaceuticals; North Wales, PA) on Day 0 and 1 U/kg of bleomycin on days 14, 28, 42 and 56 via oropharyngeal aspiration while under anesthesia. At day 70, 14 days following the final bleomycin insult, precision cut lung slices were obtained. Following euthanization, 2% low gelling temp agarose was injected into the mouse lungs via the trachea. Lungs were excised and the inferior lobe separated by dissection. The lobes were then subjected to precision slicing to obtain samples for culture using a microtome (Compresstome VF-300-0Z, Precisionary; Greenville, NC). Individual slices were distributed in a multiwell culture plate and cultured for 7 days under control (DMSO) and test compound conditions. The viability of the slices over the course of culturing was confirmed by WST-1 assay of mitochondrial activity.

During the culture period, slices in the control group were treated with DMSO and slices in the test group were treated with a DMSO solution of one of: compound 5; nintedanib; pirfenidone; a combination of nintedanib and compound 5; a combination of pirfenidone and compound 5; or the small molecule ALK5 inhibitor. Compound 5 was administered to mice effective to equal or exceed its respective IC₅₀ values at αvβ₆ and αvβ₁. The small molecule ALK5 inhibitor was analyzed at 10x its reported IC₅₀ value. Nintendanib and pirfenidone were analyzed at concentrations 10x their reported therapeutic concentrations.

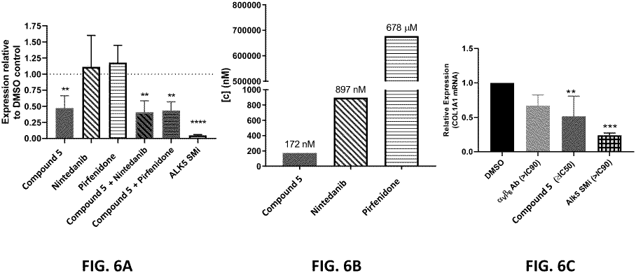

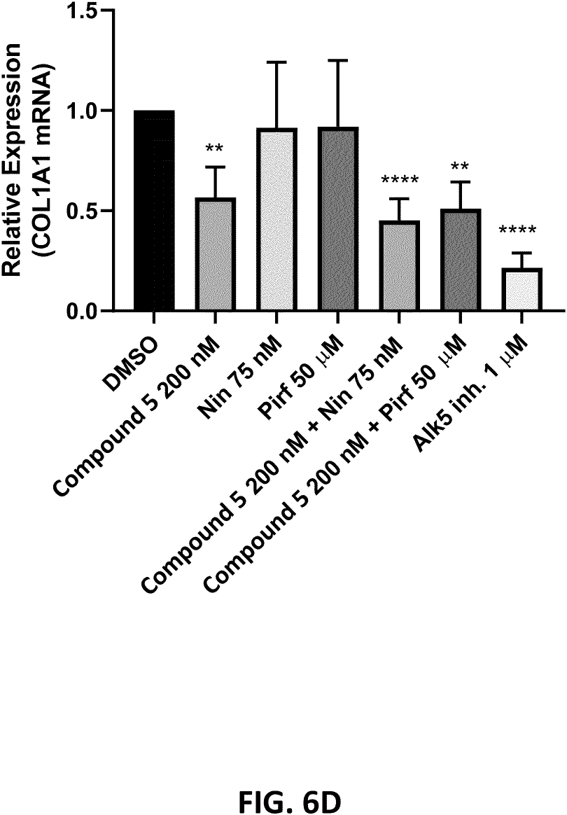

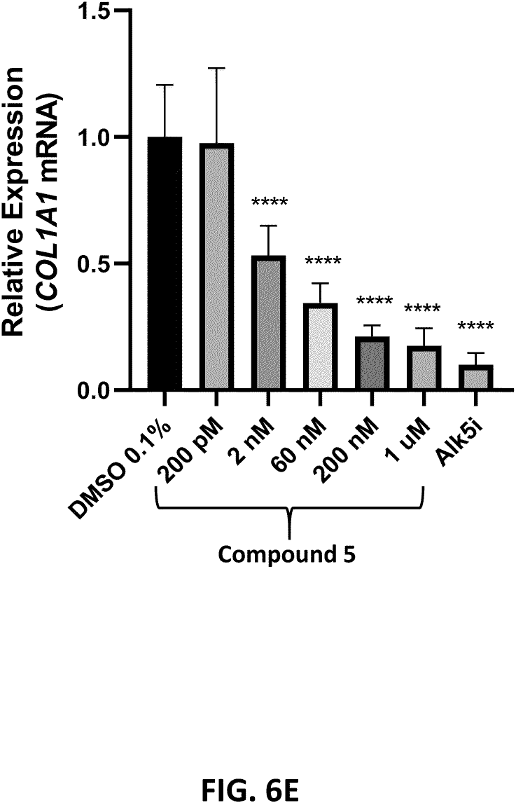

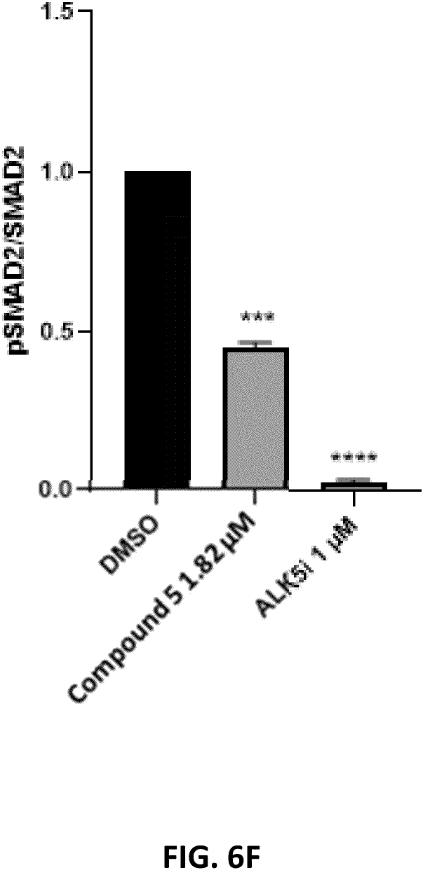

FIG. 6A is a bar graph showing that compared to the DMSO vehicle control slices, both nintedanib and pirfenidone showed a slight increase in lung Col1a1 expression, although the increase was not shown to be statistically significant. By contrast, compound 5 both alone (p<0.01 vs vehicle) and in combination with nintedanib or pirfenidone showed a substantial, statistically significant (p<0.01 vs vehicle) decrease in lung slice Col1a1 expression. Likewise, the small molecule ALK5 inhibitor, used as a positive control representative of total TGF-beta signaling inhibition, showed a substantial, statistically significant (p<0.0001 vs vehicle) decrease in lung Col1a1 expression.

FIG. 6B is a bar graph showing the concentration of compound needed to reduce lung slice Col1a1 expression by 50% compared to DMSO control slices. Data for FIG. 6B was obtained using acute bleomycin injured lung slices prepared as described in Example B8. To match the efficacy of compound 5, nintedanib required a 5.2 fold increase in concentration over compound 5, and pirfenidone required a 3,940-fold increase in concentration over compound 5.

### Example B11—Dual αᵥβ₁/αᵥβ₆ Inhibition Significantly Reduces Collagen Gene Expression in Precision Cut Lung Slices from Human IPF Explants

Explanted lung tissue was obtained from human IPF subjects and inflated with agarose as described in the preceding examples. Biopsy cores were obtained from the agarose-inflated lung tissue. The biopsy cores were subjected to precision slicing to obtain several hundred µm thick. Individual slices were distributed in a multiwell culture plate and cultured for 3 days under control (DMSO) and test compound conditions. The viability of the slices over the course of culturing was confirmed by WST-1 assay of mitochondrial activity.

During the culture period, slices in the control group were treated with DMSO and slices in the test group were treated with a DMSO solution of one of: the selective antibody αvβ₆ inhibitor, 3G9, at ≥ 400 ng/mL; compound 5, at 179 nM; and the small molecule ALK5 inhibitor at 1 µM.

FIG. 6C is a bar graph, normalized to control slices treated with DMSO, showing that all test treatments reduced lung Col1a1 expression. The selective antibody αvβ₆ inhibitor, 3G9, slightly reduced lung Col1a1 expression, but was not statistically significant. Compound 5 showed a substantial, statistically significant (p<0.01 vs vehicle) decrease in lung Col1a1 expression, as did the small molecule ALK5 inhibitor (p<0.0001 vs vehicle). Notably, in these human IPF subject samples, compound 5 was much closer in efficacy to the small molecule ALK5 inhibitor than in the murine bleomycin model.

PCLS from 5-7 idopathic pulmonary fibrosis (IPF) lung tissue samples were cultured for seven days with one of: DMSO; Compound 5 at 200 nM; nintedanib at 75 nM; pirfenidone at 50 µm; a combination of Compound 5 at 200 nM and nintedanib at 75 nM; a combination of Compound 5 at 200 nM and pirfenidone at 50 µm; or an Alk5 inhibitor at 1 µm. Compound 5 alone or in combination with nintedanib or pirfenidone reduced COL1A1 expression by 43%, 55%, and 49%, respectively. Nintedanib and pirfenidone treatment alone did not significantly reduce expression of COL1A1. FIG. 6D is a bar graph showing relative expression of COL1A1 in precision cut lung slices (PCLS) from idopathic pulmonary fibrosis (IPF) lung tissue upon exposure to Comopund 5, clinical standard of care compounds nintedanib (Nin) and pirfenidone (Pirf), and an ALK5 inhibitor, all versus DMSO control.

PCLS from a single IPF lung tissue sample were cultured for seven days with Compound 5 at concentrations of 200 pM, 2 nM, 60 nM, 200 nM, and 1 µM, along with 0.1% DMSO control and an Alk5 inhibitor at 1 µM. There was a dose dependent reduction in COL1A1 expression with a significant reduction observed ≥ 2 nM (≥ 47% reduction). FIG. 6E is a bar graph showing a dose dependent reduction of COL1A1 expression in PCLS from human IPF lung tissue upon treatment with concentrations of compound 5 ranging from 200 pM to 1 µM. COL1A1 expression is also graphed for the PCLS in the presence of 0.1% DMSO control, and an Alk5 inhibitor at 1 µM.

PCLS from 3 IPF lung tissues were cultured for seven days with Compound 5. Dual inhibition of αvβ₆ and αvβ₁ with Compound 5 significantly reduced pSMAD2/SMAD2 ratio, a marker of the canonical TGF-β signaling pathway, in PCLS by approximately 50%. FIG. 6F is a bar graph showing the effect of dual selective αvβ₆ and αvβ₁ inhibition (Compound 5 at 1.82 µM) on the ratio of pSMAD2/SMAD2 in PCLS from human IPF lung tissue samples. The ratio of pSMAD2/SMAD2 is also graphed for the PCLS in the presence of 0.1% DMSO control, and an Alk5 inhibitor at 1 µM

### Example B12—A Dual αᵥβ₁/αᵥβ₆ Inhibitor Demonstrates Good Oral Bioavailability and Pharmacokinetics in Healthy Human Subjects

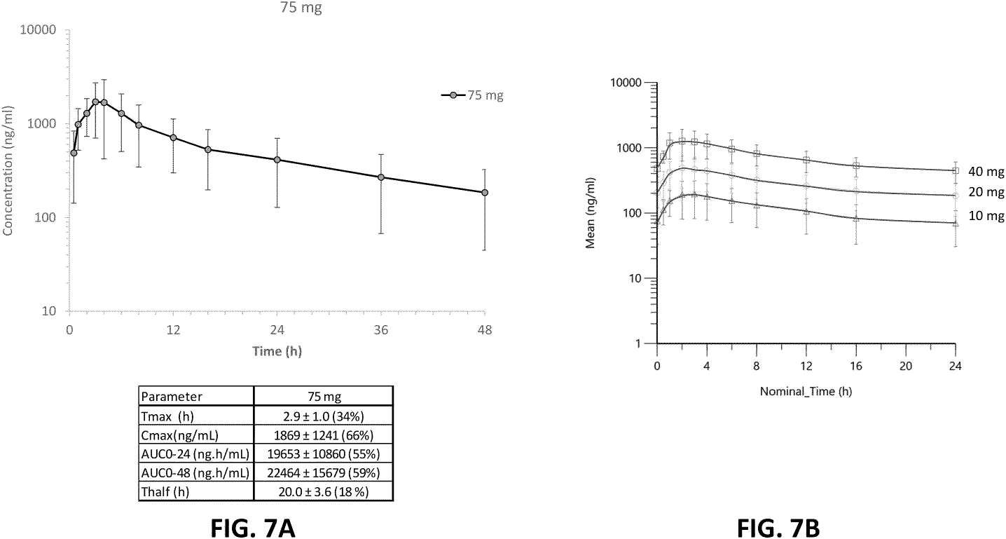

Healthy human subjects (N=85) were selected for single ascending dose (SAD) and multiple ascending dose (MAD) first-in-human studies. A solution for oral administration was prepared, containing 10 mg/mL of compound 5 in a 50:50 mixture of ORA-SWEET® SF (PERRIGO®, Allegan, Michigan) and sterile water for irrigation. Sufficient solution was administered orally to the subjects to provide between 15 mg/dose and 75 mg/dose of compound 5 in the SAD study and between 10 mg/dose and 40 mg/dose of compound 5 in the MAD study. Concentrations of compound 5 were measured in the subjects by obtaining a sample of plasma from each subject at desired intervals, and subjecting the plasma to liquid chromatography-mass spectrometry-mass spectrometry (LC-MS/MS), with quantification using a calibration curve determined from a range of solutions at standardized concentrations. The lower limit of quantitation (LLOQ) of the assay was 1 ng/mL and the calibration curve range was 1 to 500 ng/mL. FIG. 7A shows an example of the SAD study data for administration of 15, 30, 50, and 75 mg of compound 5, and further PK data for 75 mg, which is representative of the results obtained for SAD doses at 15, 30, and 50 mg. FIG. 7B shows the MAD study data for administration of 10, 20, and 40 mg of compound 5. The calculated half life of the compound varied between 18-20 hours, which supports daily administration, such as once-daily administration.

### Example B13—A Dual αᵥβ₁/αᵥβ₆ Inhibitor Demonstrates Reduction of pSMAD2/SMAD2 in BAL from Healthy Human Subjects

In order to evaluate the change of pSMAD2 as a biomarker of TGF-β activity following administration of an integrin inhibitor, and to determine a therapeutically effective dosage and an effective blood plasma Cₘₐₓ of the integrin inhibitor, healthy subjects were administered compound 5, a dual selective αvβ₆/avβ₁-integrin inhibitor, and the corresponding Cₘₐₓ levels and decrease in phosphorylation levels and were determined.

Healthy non-smoking adult males without history of lung disease were selected as subjects and were randomized into 4 cohorts. Broncoalevaolar lavage samples were obtained from all subjects 1 day prior to start of treatment. Cohorts 1 and 2 were administered 20 mg of a compound daily, wherein 3 subjects were administered the dual selective αvβ₆/αvβ₁-integrin inhibitor (compound 5) per every 1 subject receiving a placebo compound. Cohorts 3 and 4 were administered 40 mg of a compound daily, wherein 3 subjects were administered the dual selective αvβ₆/αvβ₁- integrin inhibitor (compound 5) per every 1 subject receiving a placebo compound. BAL samples and blood samples were taken from all subjects on Day -1 (baseline) and on Day 7 (end of treatment).

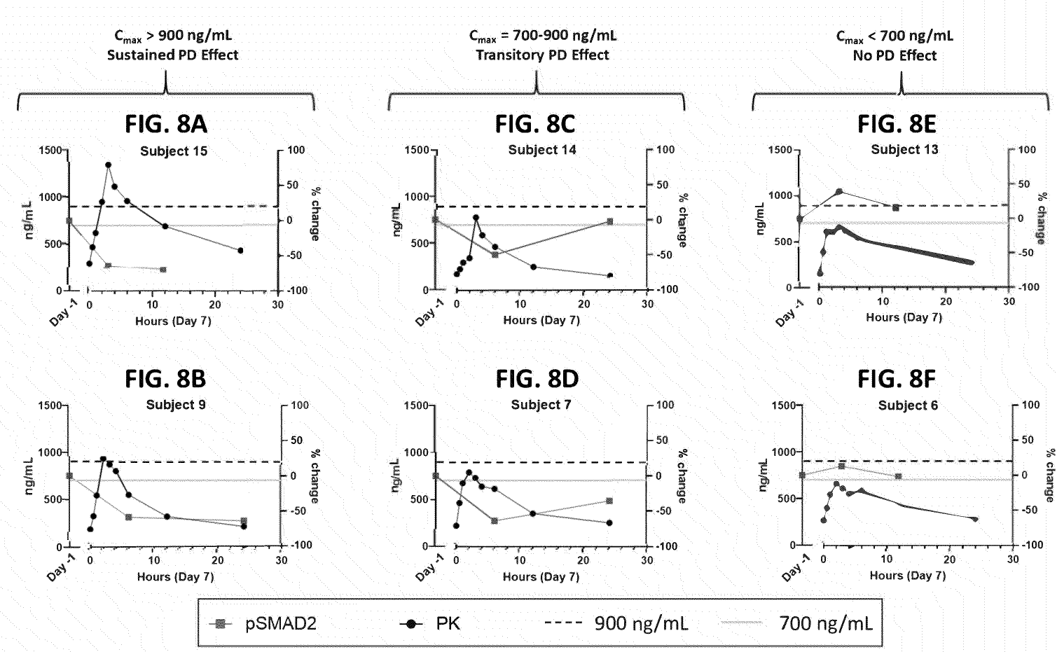

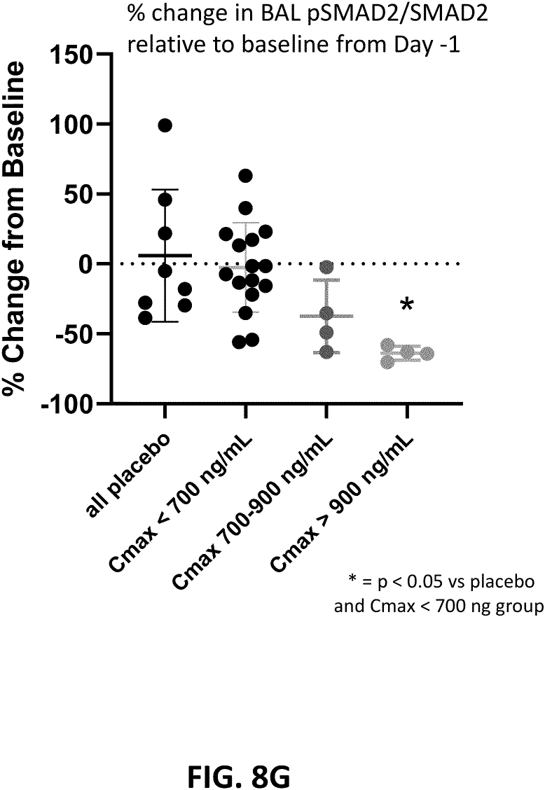

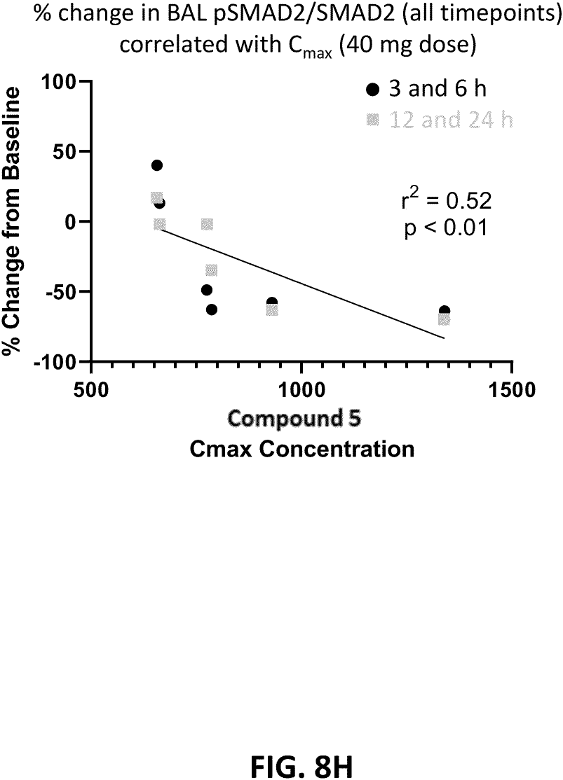

As shown in FIG. 8A, a reduction in pSMAD2:SMAD2 ratio of about 50% or more was achieved in subjects which showed higher blood plasma Cₘₐₓ of the dual selective αvβ₆/αvβ₁- integrin inhibitor (compound 5) (subjects 15, 9, 14, 7). All subjects with Cₘₐₓ above 700 ng/mL exhibited about 50% or more reduction in pSMAD2:SMAD2 ratio when compared to the placebo group (FIG. 8G) using the dual selective αvβ₆/αvβ₁ integrin inhibitor (compound 5). The Cₘₐₓ and pSMAD2:SMAD2 ratio modulations are plotted in FIG. 8H to further illustrate the relationships between dose and pSMAD2 levels. As shown in FIG. 8H, the plasma Cₘₐₓ strongly correlated with reduction of pSMAD2:SMAD2 ratio relative to baseline at 12 h and 24 h post-administration on Day 7.

### Discussion

In human and murine fibrotic lung tissue, αvβ₆ (in epithelial cells) and αvβ₁ (in fibroblasts) integrin levels are elevated and contribute to the activation of TGF-0. SMAD⅔ phosphorylation in lung tissue and BAL macrophages reflects TGF-β activation and corresponds to fibrogenic activity. SMAD⅔ phosphorylation in healthy lung tissue and BAL macrophages respond to integrin inhibitors reflecting reduced TGF-β activation. Accordingly, SMAD2 phosphorylation in BAL macrophages has been used as described herein to determine dose response and duration of inhibition of integrin inhibitors in clinical studies to establish precise PK/PD models. Dual inhibition of αvβ₆ and αvβ₁ with compound 5 also significantly reduced SMAD3 phosphorylation and fibrotic collagen deposition in the bleomycin mouse model. Dual inhibition of αvβ₆ and αvβ₁ with compound 5 significantly reduces collagen gene expression in precision cut lung slices prepared from bleomycin-injured mouse lung and from human IPF subjects. Compound 5 is comparable in antifibrotic activity to pan-αv inhibitors, and may have fewer off-target effects due to selectivity for αvβ₆ and αvβ₁. Further, dual inhibition of αvβ₆ and αvβ₁ with compound 5 is more effective than inhibition of either αvβ₆ or αvβ₁ alone. Finally, compound 5 demonstrated good oral bioavailability and pharmacokinetics in healthy subjects, offering a targeted small molecule approach for blocking TGF-β activity in pulmonary fibrosis.

### Example B14— Target Engagement of Compound 5 to αᵥβ₆ in Participants With IPF Using [18F]FP-R01-MG-F2 PET/Computerized Tomography (CT) Imaging

Integrin αvβ₆ plays a key role in promoting transforming growth factor beta activation in fibrotic diseases and can be imaged via positron emission tomography (PET) with the novel anti-αvβ₆ cystine knot peptide (knottin) radiotracer, [¹⁸F]FP-R01-MG-F2 (Kimura, et al., “Evaluation of integrin αvβ₆ cystine knot PET tracers to detect cancer and idiopathic pulmonary fibrosis,” Nature Communications (2019) 10:4673; doi:10.1038/s41467-019-11863-w.). The aim of this Example is to assess target engagement of the disclosed compounds to αvβ₆ in human participants with IPF using [¹⁸F]FP-R01-MG-F2 PET/computerized tomography (CT) imaging.

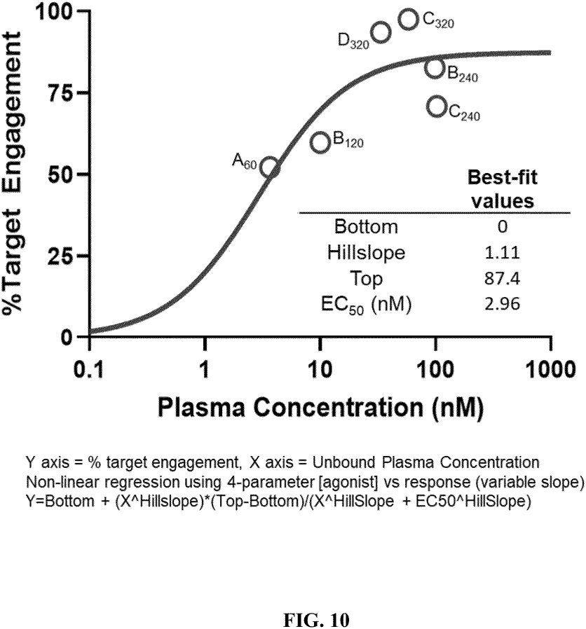

This Example was conducted as an open-label, single-dose (60 mg, 120 mg, 240 mg or 320 mg) clinical trial evaluating αvβ₆ receptor occupancy in the lungs, safety, and pharmacokinetics of Compound #5 in subjects with IPF. To date, 4 participants have completed the study. Each of these 4 participants was undergoing a pre-existing course of therapy using one of the SoC (Standard of Care) compounds, nintedanib. These participants continued their pre-existing nintedanib therapy throughout this study. Knottin tracer uptake kinetics were compared pre- and post-dose of the disclosed compound, as measured by standardized uptake values (SUVs) and parameters estimated from kinetic modeling in regions-of-interest on dynamic [¹⁸F]FP-R01-MG-F2 PET/CT scans. A two-compartment model (lung and blood) with an image-derived input function was used to fit the measured PET data (See, e.g., Peletier, et al., Impact of protein binding on receptor occupancy: a two-compartment model” J Theor Biol. 2010 Aug 21;265(4):657-71. doi: 10.1016/j.jtbi.2010.05.035). Receptor occupancy was estimated from the output of the two-compartment model (V_{T}, the volume of labeled tissue) using standard equations and fitting algorithms, and corrections for V_{ND} (non-displaceable tracer binding). For example, in FIG. 10, the data points, shown as circles, are modeled by the following formula:

$${V}_{T , p r e d} = {V}_{N D} + {V}_{S} \left(1 - \frac{C}{C + E {C}_{50}}\right)$$

to produce the depicted S curve, wherein V_{T,pred} is the predicted/fit value (S curve) of volume of labeled tissue, V_{ND} is non-displaceable binding, Vs is volume of displaceable binding, C is blood concentration of the disclosed compound, and EC₅₀ is the concentration of the disclosed compound that displaces 50% of the labeled knottin tracer.

Five subjects with IPF were enrolled: Subject A received a single dose of 60 mg of the disclosed compound followed by a post-dose scan. Subject B received two doses of the disclosed compound separated by two weeks, 120 mg and 240 mg, each followed by a post-dose scan. Subject C received two doses of the disclosed compound separated by two weeks, 240 mg and 320 mg, each followed by a post-dose scan. Subject D received a single dose of 320 mg of the disclosed compound followed by a post-dose scan. Table B-4 shows the subjects, doses, and various input and measured values for the fit.

TABLE B-4

| Subject | Dose (mg) | Conc¹ (nM) | Input Values | | Corrected For Non-Displaceable Binding (V_{NO}=0.66) | | %DeltaVt³ |
|---|---|---|---|---|---|---|---|
| | | | Baseline Vt | Vt | Baseline V₃ | Vₛ² | |
| A₀₀ | 60 | 3.66 | 3.13 | 1.84 | 2.47 | 1.18 | 52.23 |
| B₁₂₀ | 120 | 10 | 3.12 | 1.65 | 2.46 | 0.99 | 58.76 |
| B₂₄₀ | 240 | 58.3 | 3.12 | 0.72 | 2.46 | 0.06 | 97.56 |
| C₂₄₀ | 240 | 103 | 1.76 | 0.98 | 1.1 | 0.32 | 70.91 |
| C₃₂₀ | 320 | 96.5 | 176 | 0.85 | 1.1 | 0.19 | 82.73 |
| D₃₂₀ | 320 | 33.6 | 2.06 | 0.75 | 1.4 | 0.09 | 93.57 |

¹ Unbound Plasma Concentration
² Baseline Vs = (Baseline Vt) - V_{NO,} V₃= Vt - V_{NO}
³ %DeltaVT = [(Baseline Vs - Vs)/Baseline Vs] * 100

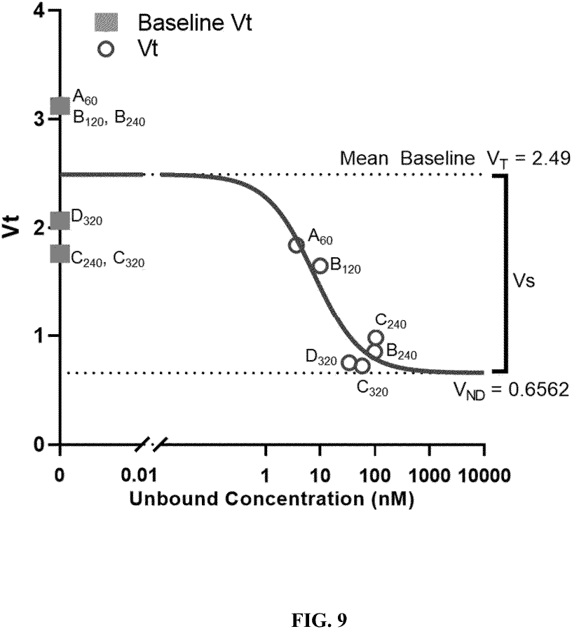

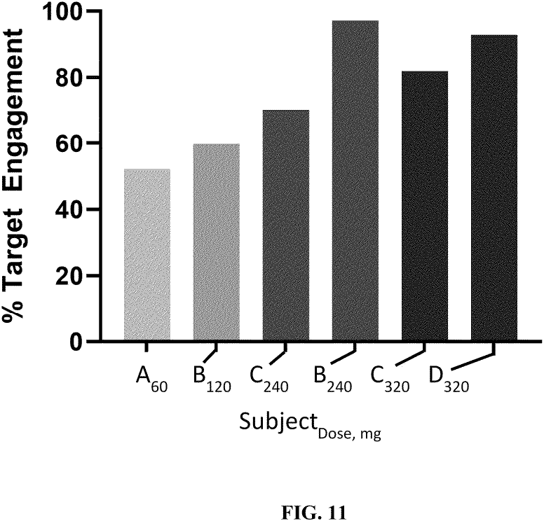

Pre-dose [¹⁸F]FP-R01-MG-F2 PET scans revealed increased αvβ₆ expression in the most fibrotic regions of the lungs. When comparing pre- and post-dose PET scans, regions with the highest αvβ₆ expression showed the most pronounced reductions in signal, as the disclosed compound displaced the knottin radiotracer. The volume of distribution of the knottin radiotracer in the lungs decreased in a dose-responsive manner, from approximately 50% in the 60 mg dose to greater than 95% in the 240 and 320 mg doses. When calculated based on measured drug exposure at 4 hours, the same pattern was observed with an exposure response saturating at a concentration of about 100 nM, and approaching 100% receptor occupancy. FIG. 9 is a graph of unbound plasma concentration (X-axis) vs Vt (Y-axis) for the baseline Vt at each dose, the measured Vt after each dose, and a fit line. FIG. 10 is a graph of unbound plasma concentration (X-axis) vs % receptor occupancy (Y-axis). FIG. 11 is a bar chart showing % target engagement for each subject and dose.

Single doses of the disclosed compound were associated with decreased knottin radiotracer accumulation in the lungs of participants with IPF. These findings suggest target engagement of the disclosed compound in IPF lungs and that the anti-αvβ₆ knottin PET radiotracer may have clinical utility as a predictive and on-treatment biomarker in IPF. Moreover, these results indicate that the effective distribution of disclosed compound throughout the lung tissue and importantly into regions of high αvβ₆ expression and high amounts of fibrosis. Receptor occupancy of >95% indicates that nearly full inhibition of TGF-β activation by αvβ₆ can be achieved at pharmacologically relevant plasma concentrations and can indicate a significant reduction in TGF-β driven fibrosis in the lung of IPF patients.

### Example B15—The Disclosed Compound is Safe and Tolerated in IPF Subjects

This Example describes a Phase 2a, multicenter, 3-part, randomized, double-blind, dose-ranging, placebo-controlled study designed to evaluate the safety, tolerability, and PK of once-daily (QD) treatment with compound 5 in vivo in human participants with idiopathic pulmonary fibrosis (IPF). Each study part will include up to 28-day screening period, a 4-week (Part A) or 12-week (Parts B and C) treatment period, and a 2-week (±3 days) posttreatment follow-up period.

Part A enrollment was completed, with 54 total participants enrolled. At the time of enrollment, 44 of these 54 participants were undergoing a pre-existing course of therapy using one of the SoC (Standard of Care) compounds (Nintedanib or Pirfenidone). It is anticipated that these participants will continue the SoC therapy throughout this study.

Part B has initiated dosing and Part C will commence following review of the clinical data supporting the evaluation of higher doses. Potential participants who provide written informed consent will be screened for study eligibility up to 28 days before administration of the first dose of compound 5.

In Parts B and C, eligible participants will be randomized on Day 1 (Visit 2). Study treatment will be administered for 12 weeks. Randomization will be stratified by use of standard of care (SoC) IPF therapy (pirfenidone or nintedanib) (SoC use; yes or no).

In Part B, 28 eligible participants will be randomized in a 3:1 ratio (active:placebo). In Part C, 2 additional compound 5 dose groups of 80 mg and 160 mg are planned for evaluation in parallel treatment groups based on the following criteria:
Part B has been completely enrolled (28 participants have randomized.)
Favorable review by the Data Safety Management Board (DSMB) of:
   All available safety and PK data from this study (Parts A and B)
   Safety and PK data from compound 5 in an ongoing Phase 1 study evaluating the safety, tolerability, and pharmacokinetics of compound 5 at multiple doses ranging from 80 to 160 mg in healthy participants

In Part C, approximately 56 eligible participants will be randomized in a 3:3:2 ratio (80 mg compound 5:160 mg compound 5:placebo) and treated for 12 weeks in parallel treatment groups. The total number of participants enrolled in Parts B and C of the study will be approximately 84, with approximately 63 receiving compound 5 and 21 receiving placebo.

Participants who discontinue study drug for safety reasons prior to completion of 12 weeks of treatment will be encouraged to remain in the study to complete all remaining assessments. Where this is not feasible, the subject will be asked to return to the clinic for an Early Termination (ET) visit for follow-up evaluations.

Participant safety will be assessed at predetermined intervals during the study, including evaluation of all safety and PK data to enable initiation of Part C, and as needed. Participants will also be assessed for any adverse events.

Initial results of the study through a twelve-week treatment period are described in Example B15A.

### Example B15A—Phase 2a Clinical Trial

The randomized, double-blind, placebo-controlled Phase 2a clinical trial of Compound 5 in patients with idiopathic pulmonary fibrosis (IPF) as described in Example B15 was initiated. The trial met its primary and secondary endpoints demonstrating that Compound 5 was well tolerated over a 12-week treatment period and displayed a favorable pharmacokinetic profile. The trial’s exploratory endpoint assessing forced vital capacity (FVC), showed a dose-dependent treatment effect on FVC versus placebo at 12 weeks in Compound 5 treated patients. A dose-dependent reduction was observed in the proportion of patients experiencing a FVCpp decline of ≥10%.

Compound 5 was evaluated at once-daily doses of 40 mg, 80 mg, 160 mg or placebo for 12 weeks in 90 patients with IPF. 67 patients were enrolled in the active arms and 23 patients were enrolled in the placebo arm. Approximately 80% of the enrolled patients were on standard of care and were equally distributed between nintedanib and pirfenidone.

At all three doses tested, Compound 5 was well tolerated. Of the 67 patients treated with Compound 5, 65 (97%) completed 12 weeks of treatment with no discontinuations due to adverse events. No treatment related deaths or drug related serious adverse events (SAE) were reported. Most treatment emergent adverse events (TEAE) were mild or moderate in severity.

Compound 5 exhibited generally dose proportional increases in plasma concentrations, consistent with prior studies.

The exploratory endpoints of the trial measured changes in forced vital capacity (FVC), HRCT-based Quantitative Lung Fibrosis score (QLF), and selected biomarkers over 3 months of treatment.

A treatment effect was observed in all Compound 5 dose groups with and without standard of care therapy. A pooled analysis of Compound 5 treated patients showed an approximately 80% reduction in FVC decline over 12 weeks versus placebo (-15.1 mL for Compound 5 pooled groups versus -74.1 mL for placebo). The 40 mg and 160 mg dose groups demonstrated 38% (-46.1 mL) and 66% (-25.1 mL) reductions in FVC decline, respectively, relative to placebo. Importantly, for the 80 mg treatment group, a +24.6 mL increase in FVC was observed relative to baseline.

At 12 weeks, the proportion of patients who experienced a > 2% increase in QLF was lowest in the 80 mg group (11%). The proportion of patients who remained stable (-2 to 2% change) or experienced a decrease in QLF (>2% change) were similar in the 160 mg group (46.6% and 26.7%, respectively) and the placebo (47.1% and 23.5%, respectively), where the approximately 80% of patients received standard of care (SoC). A treatment effect of Compound 5 is suggested with a greater proportion of patients with a decrease or stable QLF score compared to placebo group. Changes in QLF (%) correlate with changes in FVC (mL) and FVCpp.

A decline of≥10% in predicted FVC (FVCpp) at 12 weeks is associated with an increased risk of death in IPF patients over a two-year period (Patemiti MO, et al Ann Am Thorac Soc. 2017 Sep;14(9): 1395-1402). The proportion of patients that experienced a ≥10% decline in FVCpp were 8.7% in the 80 mg group and 4.5% in the 160 mg group versus 17.4% in the placebo group. The 40 mg group experienced a 18.2% decline relative to placebo. The dose-dependent decrease in the proportion of patients with FVCpp decline of ≥10% suggests a potentially disease-modifying effect of Compound 5.

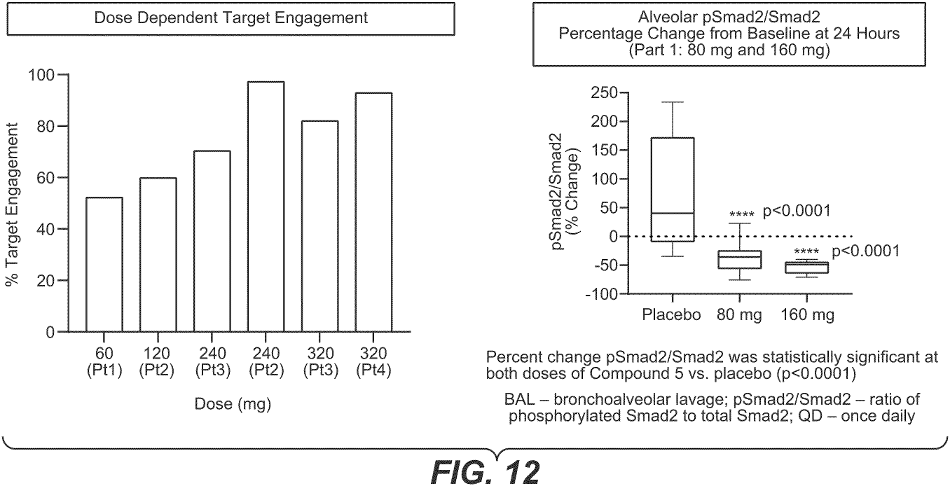

FIG. 12 through FIG. 36 illustrate various results from the clinical trial. FIG. 12 shows that Compound 5 achieved dose dependent target engagement and TGF-β suppression and in prior studies. The left graph shows Dose-Dependent Target Engagement, while the right graph shows Alveolar pSmad2/Smad2, Percentage Change from Baseline at 24 Hours (Part 1: 80 mg and 160 mg). The percent change of pSmad2/Smad2 was statistically significant at both doses of Compound 5 vs. placebo (p<0.0001). BAL - bronchoalveolar lavage; pSmad2/Smad2 - ratio of phosphorylated Smad2 to total Smad2; QD - once daily.

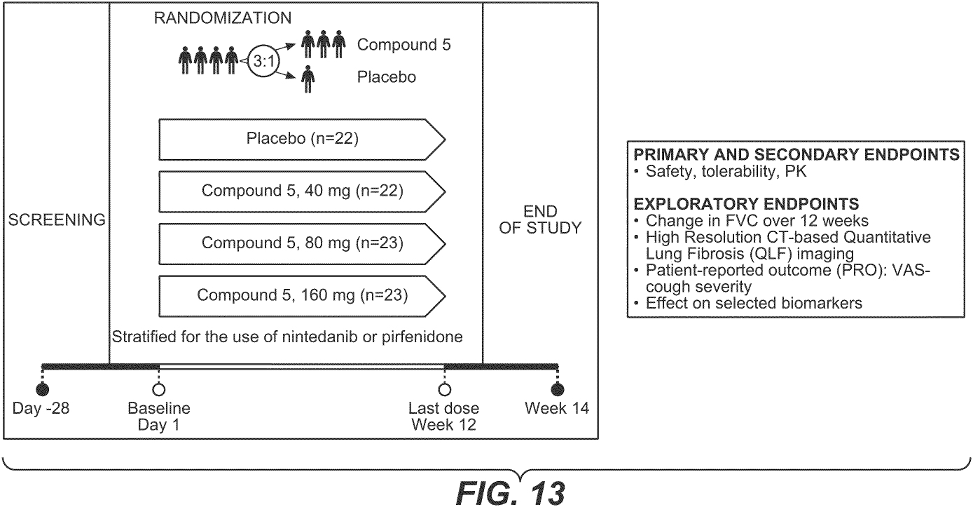

FIG. 13 shows the Study Design and Objectives. The left graph illustrates the randomized groups into which the study group population was divided: Placebo (n=22); Compound 5, 40 mg (n=22); Compound 5, 80 mg (n=23); Compound 5, 160 mg (n=23). The groups were stratified for the use of nintedanib or pirfenidone. The right graph shows the primary and secondary endpoints of the study, namely, safety, tolerability, and PK; and the exploratory endpoints: Change in FVC over 12 weeks; High Resolution CT-based Quantitative Lung Fibrosis (QLF) imaging; Patient-reported outcome (PRO): VAS-cough severity; Effect on selected biomarkers.

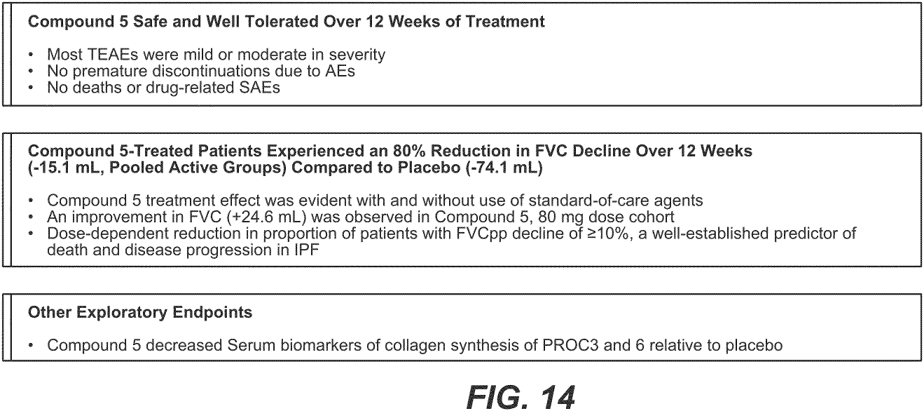

FIG. 14 shows a summary of study results. Compound 5 was safe and well tolerated over 12 weeks of treatment. Most treatment-emergent adverse events (TEAEs) were mild or moderate in severity. There were no premature discontinuations due to adverse events, and no deaths or drug-related significant adverse events. Compound 5-treated patients experienced an 80% reduction in FVC decline over 12 weeks (-15.1 mL, Pooled Active Groups) compared to placebo (-74.1 mL). Compound 5 treatment effect was evident with and without use of standard-of-care agents. An improvement in FVC (+24.6 mL) was observed in the Compound 5, 80 mg dose cohort. There was a dose-dependent reduction in proportion of patients with FVCpp decline of ≥10%, a well-established predictor of death and disease progression in idiopathic pulmonary fibrosis (IPF). With respect to other exploratory endpoints, Compound 5 decreased serum biomarkers of collagen synthesis of PROC3 and 6 relative to placebo.

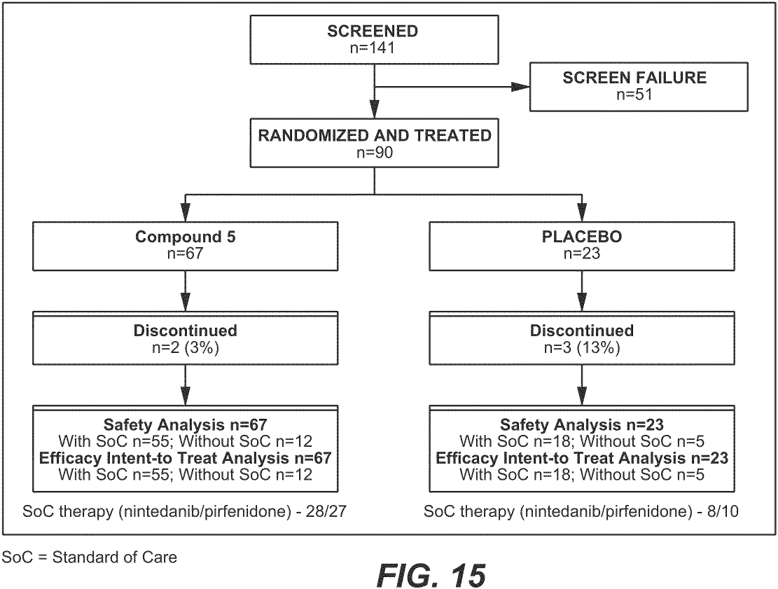

FIG. 15 shows the Participant Disposition of the study population. Firstly, a total of 141 (n=141) participants were screened with 51 participants failed the screening (n=51). The remaining 90 participants (n=90) were randomized and divided into two groups, with one group of 67 participants (n=67) being treated with Compound 5 and the other group of 23 participants (n=23) being treated with placebo. For the Compound 5 group, 67 participants (n=67) were treated with Compound 5 with the treatment for 2 participants (n=2, 3%) later being discontinued. Among these 67 participants, 55 participants (n=55) received Standard of Care (SoC) therapy while the other 12 participants (n=12) did not receive SoC therapy. And among those 55 participants who received SoC, 28 of them received nintedanib and 27 of them received pirfenidone. Safety Analysis and Efficacy Intent-To Treat Analysis were conducted for the first group with 67 participants. For the placebo group, 23 participants (n=23) were treated with placebo with the treatment for 3 participants (n=3, 13%) later being discontinued. Among these 23 participants, 18 participants (n=18) received Standard of Care (SoC) therapy while the other 5 participants (n=5) did not receive SoC therapy. And among those 18 participants who received SoC, 8 of them received nintedanib and 10 of them received pirfenidone. Safety Analysis and Efficacy Intent-To Treat Analysis were conducted for the placebo group with 23 participants.

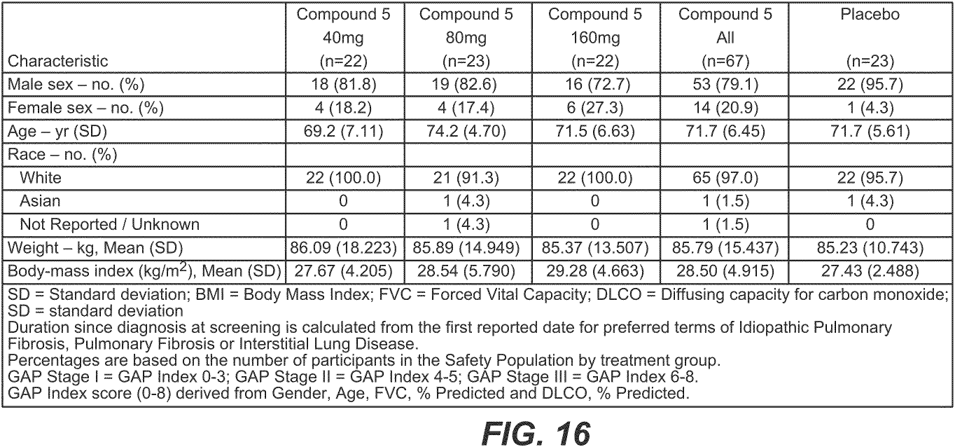

FIG. 16 shows Baseline Demographics of the study population. In the present study, 22 participants were treated with 40 mg Compound 5, 23 participants were treated with 80 mg Compound 5, 22 participants were treated with 160 mg Compound 5, and 23 participants were treated with placebo. The detailed characteristics of the participants (e.g., sex, age, race, weight, body-mass index) are summarized in FIG. 16. In FIG. 16, SD refers to Standard deviation. BMI refers to Body Mass Index. FVC refers to Forced Vital Capacity. DLCO refers to Diffusing capacity for carbon monoxide.

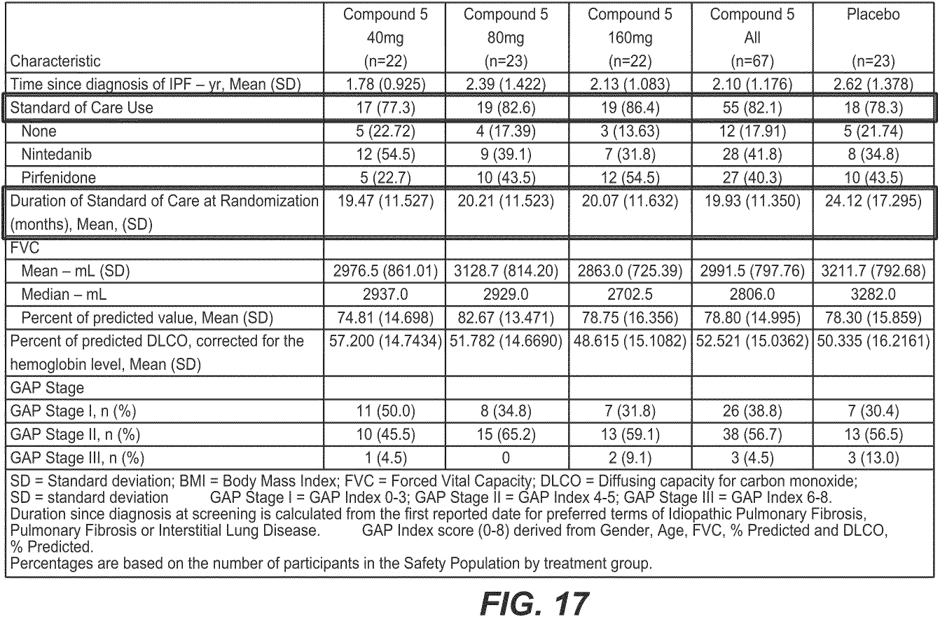

FIG. 17 shows the Baseline Disease Characteristics of the study population. In the present study, 22 participants were treated with 40 mg Compound 5, 23 participants were treated with 80 mg Compound 5, 22 participants were treated with 160 mg Compound 5, and 23 participants were treated with placebo. For these participants, duration since diagnosis at screening was calculated from the first reported date for preferred terms of Idiopathic Pulmonary Fibrosis,Pulmonary Fibrosis or Interstitial Lung Disease. GAP Index score (0-8) were derived from Gender, Age, FVC, % Predicted and DLCO, % Predicted. Percentages were calculated based on the number of participants in the Safety Population by treatment group. The detailed characteristics, especially the diseases characteristics of the participants (e.g., time since diagnosis of IPF, Standard of Care Use, Duration of Standard of Care at Randomization, FVC, Gap Stage, etc.) are summarized in FIG. 17. GAP Stage I = GAP Index 0-3; GAP Stage II = GAP Index 4-5; GAP Stage III = GAP Index 6-8. GAP Index score (0-8) is derived from Gender, Age, FVC, % Predicted and DLCO, % Predicted. In FIG. 17, SD refers to Standard deviation. BMI refers to Body Mass Index. FVC refers to Forced Vital Capacity. DLCO refers to Diffusing capacity for carbon monoxide.

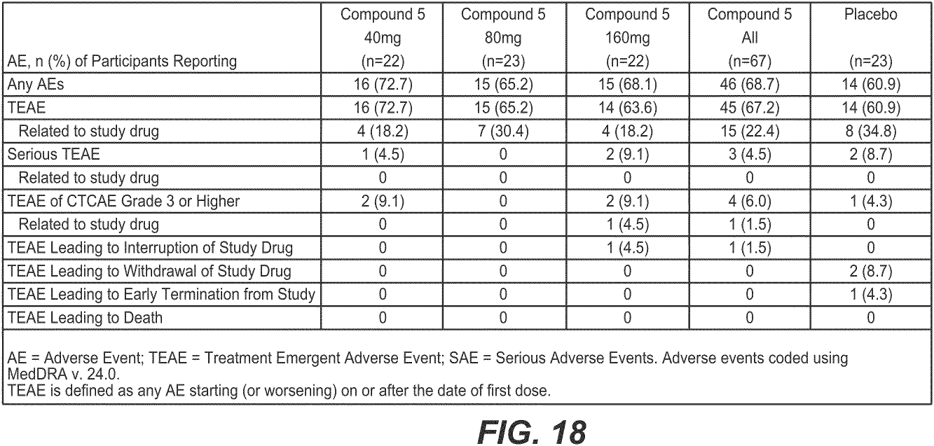

FIG. 18 shows the Overall Safety Summary. In the present study, 22 participants were treated with 40 mg Compound 5, 23 participants were treated with 80 mg Compound 5, 22 participants were treated with 160 mg Compound 5, and 23 participants were treated with placebo. The number and percentage of participants who report adverse event (AE), level of adverse event, and actions followed by adverse events (e.g., interruption of Study Drug, withdrawal of Study Drug, early termination from study, etc.) are summarized in FIG. 18. In FIG. 18, AE refers to Adverse Event. TEAE refers to Treatment Emergent Adverse Event. SAE refers to Serious Adverse Events. Adverse events were coded using MedDRA v. 24.0. TEAE is defined as any AE starting (or worsening) on or after the date of first dose.

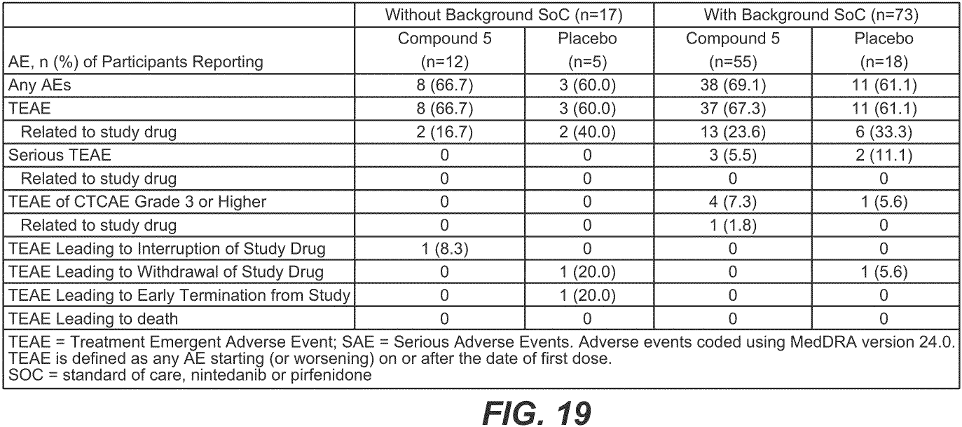

FIG. 19 shows the Overall Safety Summary by SOC Use in Pooled Compound 5 Groups. Among all participants, 17 were treated without Background SoC and 73 were treated with Background SoC. The number and percentage of participants who report adverse event (AE), level of adverse event, and actions followed by adverse events (e.g., interruption of Study Drug, withdrawal of Study Drug, early termination from study, etc.) are summarized in FIG. 19. In FIG. 19, TEAE refers to Treatment Emergent Adverse Event. SAE refers to Serious Adverse Events. SoC refers to standard of care, with nintedanib or pirfenidone. TEAE is defined as any AE starting (or worsening) on or after the date of first dose. Adverse events were coded using MedDRA version 24.0.

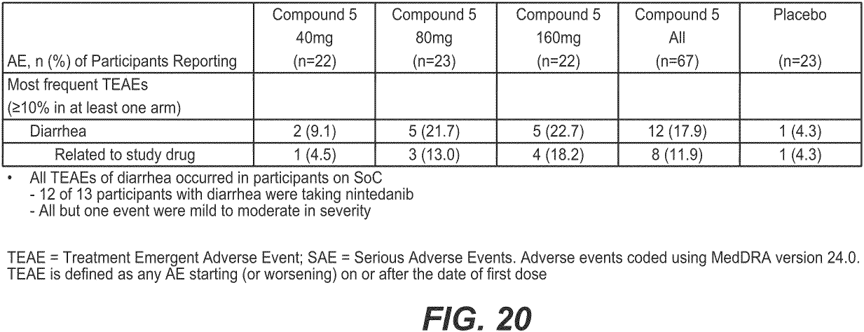

FIG. 20 shows the Most Frequent TEAEs - Any Causality. All TEAEs of diarrhea occurred in participants on SoC. 12 of 13 participants with diarrhea were taking nintedanib. All but one event were mild to moderate in severity. In FIG. 20, TEAE refers to Treatment Emergent Adverse Event. SAE refers to Serious Adverse Events. TEAE is defined as any AE starting (or worsening) on or after the date of first dose. Adverse events were coded using MedDRA version 24.0.

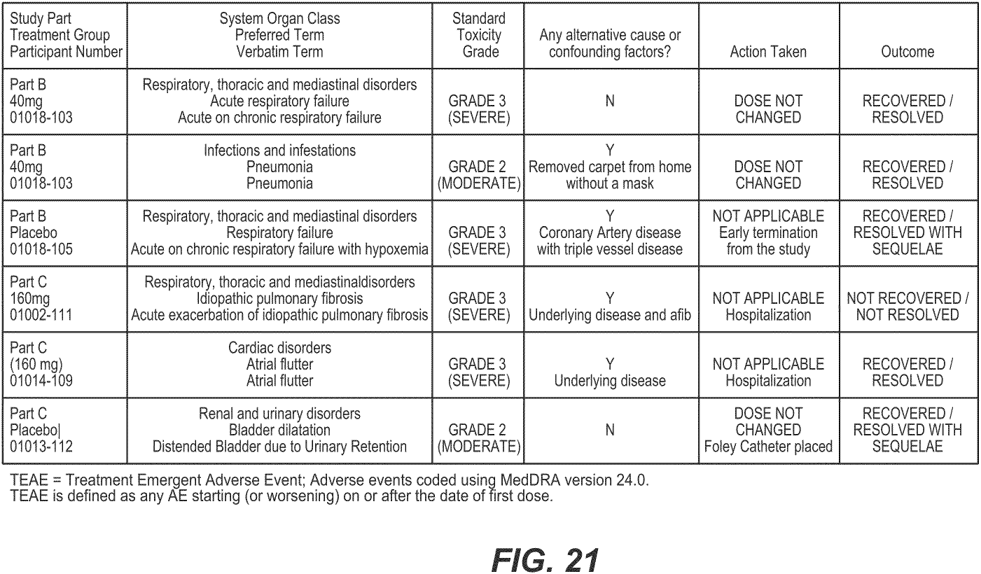

FIG. 21 shows the No Treatment-Emergent SAEs Related to Study Drug. In FIG. 21, TEAE refers to Treatment Emergent Adverse Event. SAE refers to Serious Adverse Events. TEAE is defined as any AE starting (or worsening) on or after the date of first dose.

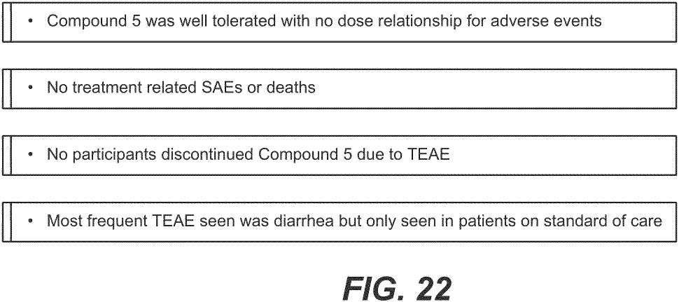

FIG. 22 shows the Overall Summary of Safety Evaluation. Compound 5 was well tolerated with no dose relationship for adverse events. No treatment related SAEs or deaths happened. No participants were discontinued the treatment of Compound 5 due to TEAE. Most frequent TEAE seen was diarrhea but only seen in patients on standard of care.

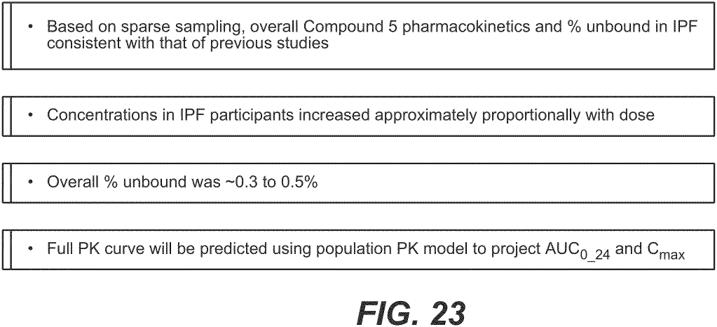

FIG. 23 shows the Overall Summary of Pharmacokinetics. Based on sparse sampling, overall Compound 5 pharmacokinetics and % unbound in IPF were consistent with that of previous studies. Concentrations in IPF participants increased approximately proportionally with dose. Overall % unbound was ~0.3 to 0.5%. Full PK curve will be predicted using population PK model to project AUC_{0_24} and C_{max.}

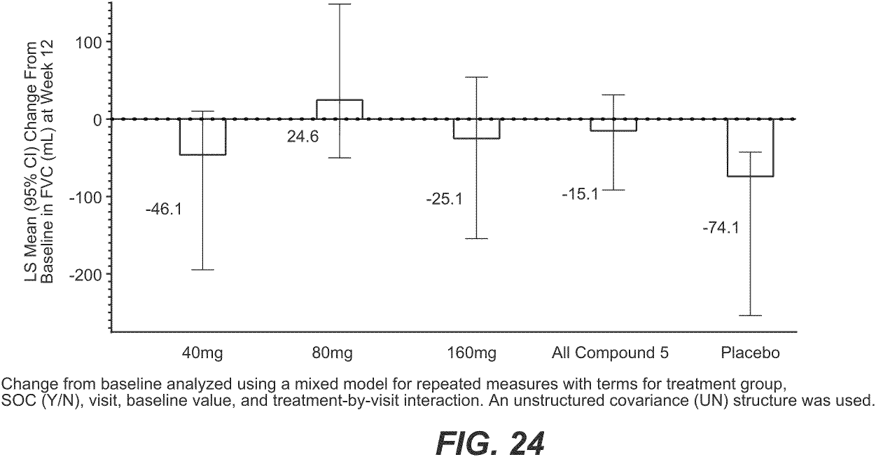

FIG. 24 shows the Change in FVC from Baseline to Week 12 (MMRM Analysis, ITT Population). Data shown from left to right is participants treatment with 40 mg Compound 5, 80 mg Compound 5, 160 mg Compound 5, all participants treated with Compound 5, and participants treated with Placebo. Change from baseline was analyzed using a mixed model for repeated measures with terms for treatment group, SOC (Y/N), visit, baseline value, and treatment-by-visit interaction. An unstructured covariance (UN) structure was used.

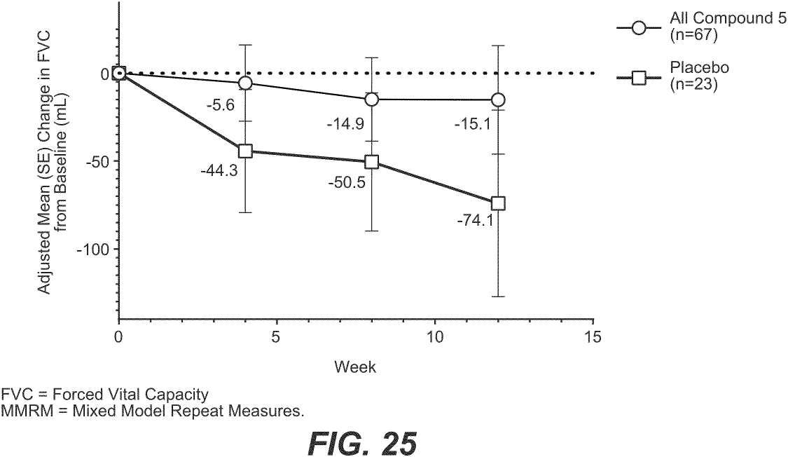

FIG. 25 shows the Change in FVC over Time in Pooled Compound 5 Groups (MMRM Analysis, ITT Population). Data trace with circles represents all participants treated with Compound 5 (n=67) and data trace with squares represents all participants treated with Placebo (n=23). FVC refers to Forced Vital Capacity. MMRM refers to Mixed Model Repeat Measures.

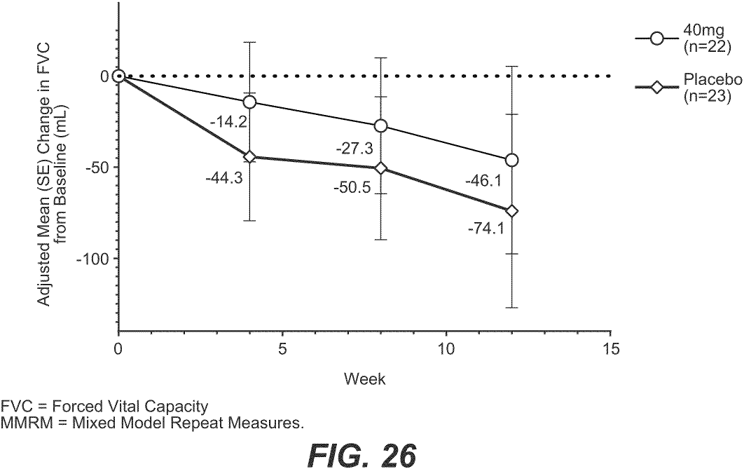

FIG. 26 shows the Change in FVC over Time in 40 mg Compound 5 Group (MMRM Analysis, ITT Population). Data trace with circles represents participants treated with 40 mg Compound 5 (n=22) and data trace with diamond represents all participants treated with Placebo (n=23). FVC refers to Forced Vital Capacity. MMRM refers to Mixed Model Repeat Measures.

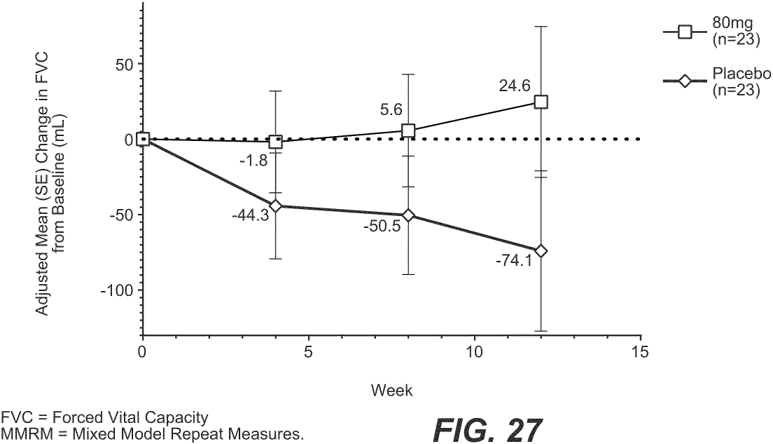

FIG. 27 shows the Change in FVC over Time in 80 mg Compound 5 Group (MMRM Analysis, ITT Population). Data trace with squares represents all participants treated with 80 mg Compound 5 (n=23) and data trace with diamonds represents all participants treated with Placebo (n=23). FVC refers to Forced Vital Capacity. MMRM refers to Mixed Model Repeat Measures.

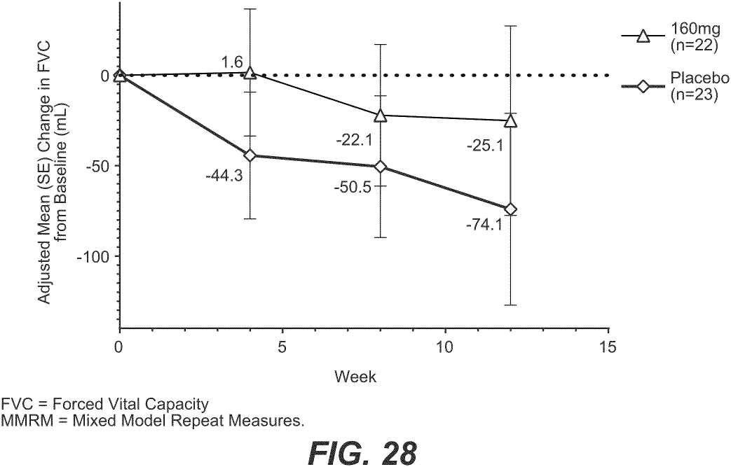

FIG. 28 shows the Change in FVC over Time in 160 mg Compound 5 Group (MMRM Analysis, ITT Population). Data trace with triangles represents all participants treated with 160 mg Compound 5 (n=22) and data trace with diamonds represents all participants treated with Placebo (n=23). FVC refers to Forced Vital Capacity. MMRM refers to Mixed Model Repeat Measures.

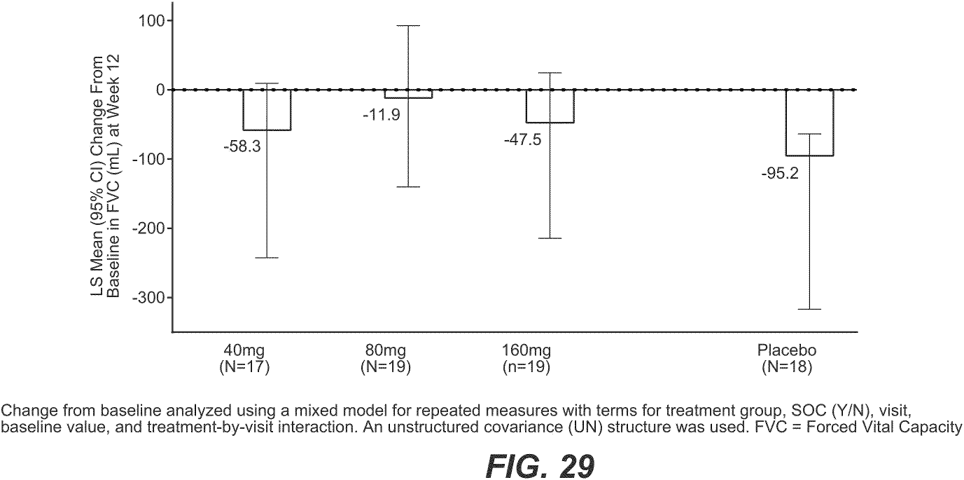

FIG. 29 shows the Change in FVC from Baseline to Week 12 in On SoC Subgroup (MMRM Analysis, ITT Population). As shown in FIG. 29, participants treated with 40 mg Compound 5 (N=17) showed a LS Mean (95% Cl) Change From Baseline in FVC (mL) at week of 12 of -58.3 mL, participants treated with 80 mg Compound 5 (N=19) showed a LS Mean (95% Cl) Change From Baseline in FVC (mL) at week of 12 of -11.9 mL, participants treated with 160 mg Compound 5 (n=19) showed a LS Mean (95% Cl) Change From Baseline in FVC (mL) at week of 12 of -47.5 mL, and participants treated with Placebo (N=18) showed a LS Mean (95% Cl) Change From Baseline in FVC (mL) at week of 12 of -95.2 mL. Change from baseline was analyzed using a mixed model for repeated measures with terms for treatment group, SOC (Y/N), visit, baseline value, and treatment-by-visit interaction. An unstructured covariance (UN) structure was used. FVC refers to Forced Vital Capacity.

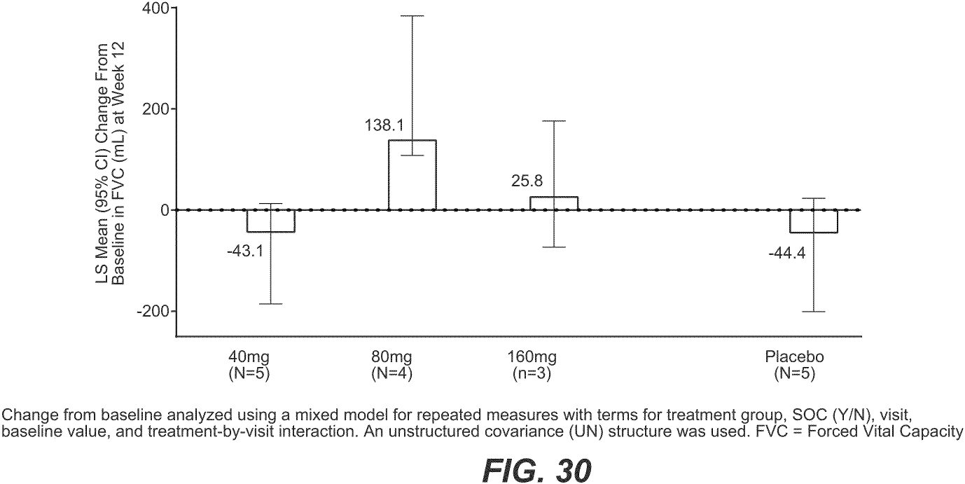

FIG. 30 shows the Change in FVC from Baseline to Week 12 in Not on SoC Subgroup (MMRM Analysis, ITT Population). As shown in FIG. 30, participants treated with 40 mg Compound 5 (N=5) showed a LS Mean (95% Cl) Change From Baseline in FVC (mL) at week of 12 of -43.1 mL, participants treated with 80 mg Compound 5 (N=4) showed a LS Mean (95% Cl) Change From Baseline in FVC (mL) at week of 12 of +138.1 mL, participants treated with 160 mg Compound 5 (n=3) showed a LS Mean (95% Cl) Change From Baseline in FVC (mL) at week of 12 of +25.8 mL, participants treated with Placebo (N=5) showed a LS Mean (95% Cl) Change From Baseline in FVC (mL) at week of 12 of -44.4 mL. Change from baseline was analyzed using a mixed model for repeated measures with terms for treatment group, SOC (Y/N), visit, baseline value, and treatment-by-visit interaction. An unstructured covariance (UN) structure was used. FVC refers to Forced Vital Capacity.

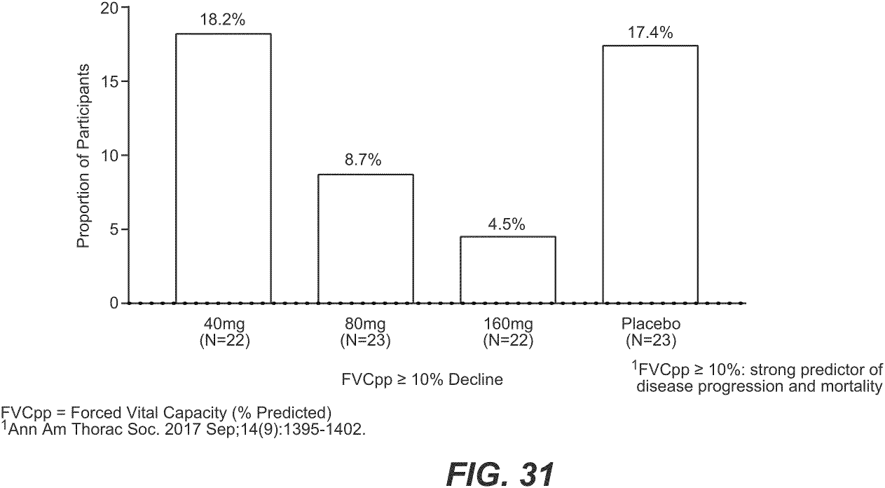

FIG. 31 shows the Proportion of Participants with FVCpp Decline ≥10% with ITT Population. As shown in FIG. 31, participants treated with 40 mg Compound 5 (N=22) represent 18.2% of total participants, participants treated with 80 mg Compound 5 (N=23) represent 8.7% of total participants, participants treated with 160 mg Compound 5 (N=22) represents 4.5% of total participants, participants treated with Placebo (N=23) represent 17.4% of total participants. FVCpp ≥ 10% is a strong predictor of disease progression and mortality wherein FVCpp refers to Forced Vital Capacity (% Predicted), according to Ann Am Thorac Soc. 2017 Sep;14(9): 1395-1402.

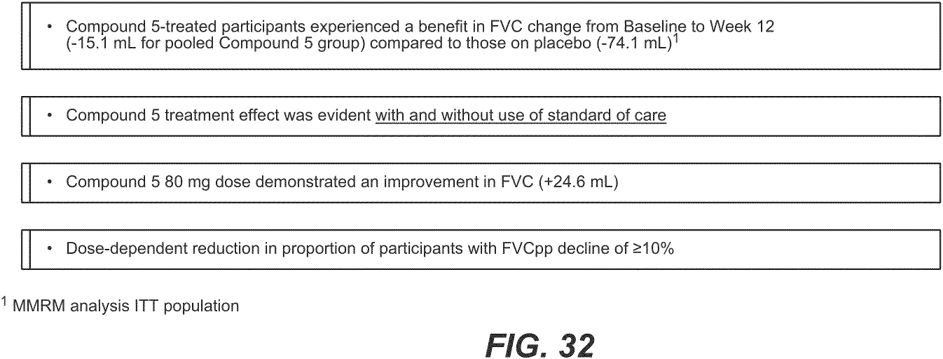

FIG. 32 shows the Overall Summary of Spirometry Evaluation. Compound 5-treated participants experienced a benefit in FVC change from Baseline to Week 12 (-15.1 mL for pooled Compound 5 group) compared to those on placebo (-74.1 mL), according to MMRM analysis with ITT population. Compound 5 treatment effect was evident with and without use of standard of care. Compound 5 80 mg dose demonstrated an improvement in FVC (+24.6 mL). There was dose-dependent reduction in proportion of participants with FVCpp decline of >10%.

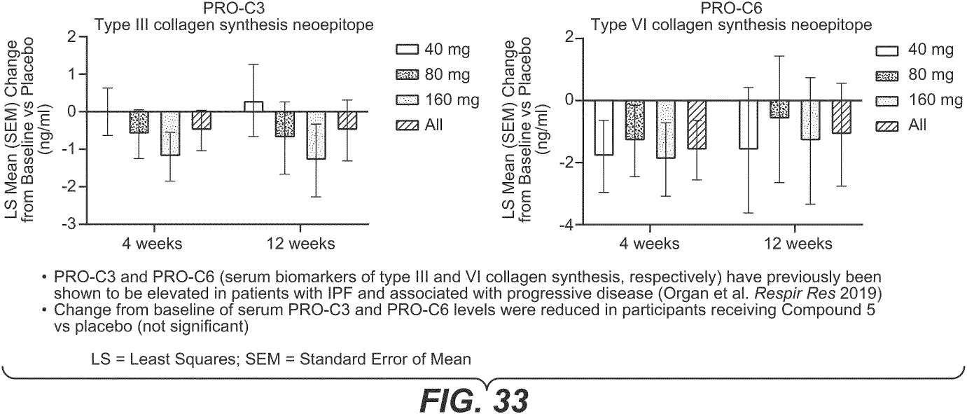

FIG. 33 shows the Compound 5 Decreased Serum Biomarkers of Collagen Synthesis Relative to Placebo. The left graph shows PRO-C3, Type III collagen synthesis neoepitope, while the right graph shows PRO-C6, Type VI collagen synthesis neoepitope. Blank filled columns represent 40 mg Compound 5 dosage. Dense dot filled columns represent 80 mg Compound 5 dosage. Light dot filled columns represent 160 mg Compound 5 dosage. Diagonal line filled columns represent all participants dosed with Compound 5. PRO-C3 and PRO-C6 (serum biomarkers of type III and VI collagen synthesis, respectively) have previously been shown to be elevated in patients with IPF and associated with progressive disease (Organ et al. Respir Res 2019). As shown in FIG. 33, change from baseline of serum PRO-C3 and PRO-C6 levels were reduced in participants receiving Compound 5 vs placebo (not significant). In FIG. 33, LS refers to Least Squares and SEM refers to Standard Error of Mean.

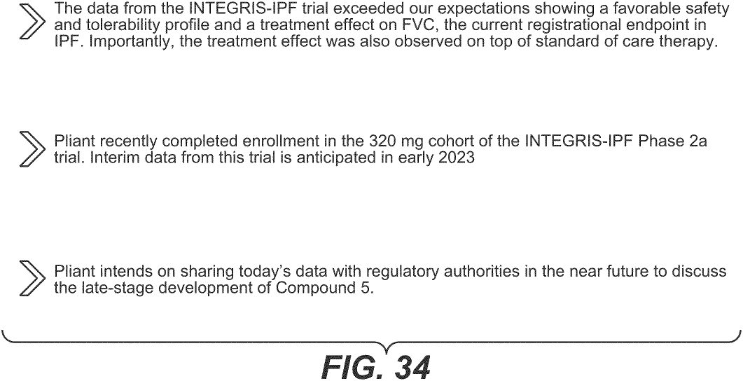

FIG. 34 shows the Conclusion and Next Steps. The data from the INTEGRIS-IPF trial exceeded our expectations, showing a favorable safety and tolerability profile and a treatment effect on FVC, the current registrational endpoint in IPF. Importantly, the treatment effect was also observed on top of standard of care therapy. Pliant recently completed enrollment in the 320 mg cohort of the INTEGRIS-IPF Phase 2a trial. Interim data from this trial is anticipated in early 2023. Pliant intends on sharing today’s data with regulatory authorities in the near future to discuss the late-stage development of Compound 5.

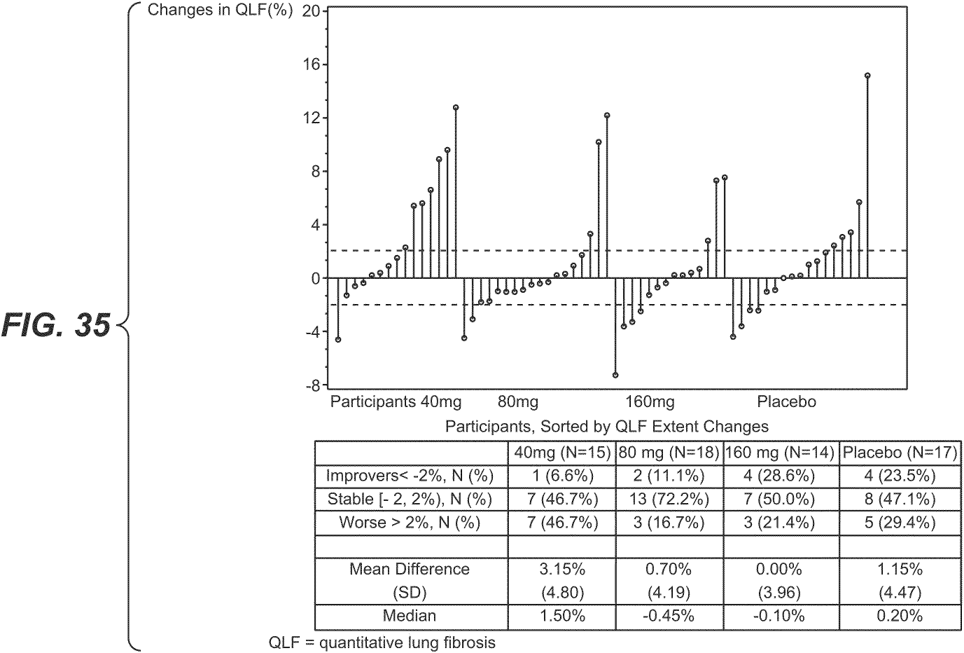

FIG. 35 shows the Mean Percent Change in quantitative lung fibrosis (QLF) Extent From Baseline to Week 12 (CT Protocol Population). The Mean Percent Change of participants, sorted by QLF extent changes were summarized in the table in FIG. 35.

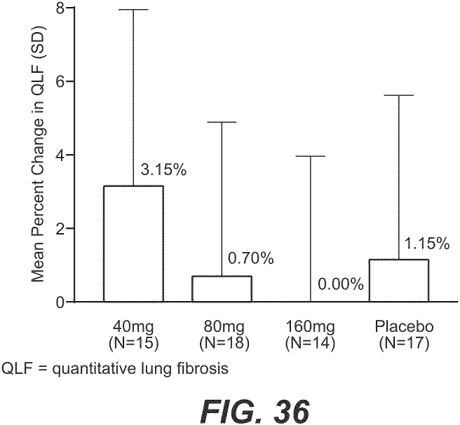

FIG. 36 shows the Mean Percent Change in quantitative lung fibrosis (QLF) Extent From Baseline to Week 12 (CT Protocol Population within Screening Window). As shown in FIG. 36, participants treated with 40 mg Compound 5 (N=15) showed a Mean Percent Change in QLF (SD) of 3.15%. Participants treated with 80 mg Compound 5 (N=18) showed a Mean Percent Change in QLF (SD) of 0.70%. Participants treated with 160 mg Compound 5 (N=14) showed a Mean Percent Change in QLF (SD) of 0.00%. Participants treated with Placebo (N=17) showed a Mean Percent Change in QLF (SD) of 1.15%.

### Example B16--The Disclosed Compound Will Inhibit Type 1 Collagen in IPF Patients

This is a Phase 2a, single-center, randomized, double-blinded, placebo-controlled study to evaluate type 1 collagen deposition in the lungs in vivo in human participants with IPF following once-daily (QD) treatment with 160 mg compound 5 for 12 weeks. Enrollment of participants is targeted to start in late 2021.

The study includes up to 28-day screening period, a 12-week treatment period, and a 2 week (±3 days) post treatment follow-up period.

Potential participants who provide written informed consent will be screened for study eligibility up to 28 days before administration of the first dose of study drug. Approximately 12 eligible participants will be randomized in a 2:1 ratio (160 mg compound 5 vs placebo; 8 receiving compound 5 and 4 receiving placebo) on Day 1 (Visit 3). Study treatment will be administered once daily for 12 weeks. Randomization will be stratified by use of standard of care (SoC) IPF therapy with pirfenidone or nintedanib (SoC use; yes or no).

A peptide-based positron emission tomography (PET) probe, ⁶⁸Ga-CBP8, that targets collagen type I will be administered (Desogere, P. et al., “Type I collagen-targeted PET probe for pulmonary fibrosis detection and staging in preclinical models,” Sci TranslMed. 2017 April 05; 9(384): doi:10.1126/scitranslmed.aaf4696.). ⁶⁸Ga-CBP8 PET/MRI scans will be conducted within 7 days prior to baseline and at or within 7 days prior to Week 12.

Participants who discontinue study drug for safety reasons prior to completion of 12 weeks of treatment will be encouraged to remain in the study to complete all remaining assessments. Where this is not feasible, the subject will be asked to return to the clinic for an Early Termination (ET) visit for follow-up evaluations. If a participant elects to withdraw from the study after the 6^{th} week of randomization, an end of participation ⁶⁸Ga-CBP8 PET/MRI will be offered to the participant to enhance appropriate data capture.

Participant safety will be assessed at predetermined intervals during the study, including evaluation of all safety and PK data to enable initiation of Part C, and as needed. Participants will also be assessed for any adverse events.

### Example B17

Fibrosis-related gene expression in explanted human lung tissue from patients with idiopathic pulmonary fibrosis was examined in order to determine what impact combining Compound 5 treatment with standard-of-care drugs nintedanib or pirfenidone, has on the expression of such genes.

### Methods

Samples used for this analysis are a subset (n = 4 of 7) of the lungs previously reported on in Decaris et al., Respir Res (2021) 22:265 (FIGS. 6 A and B). Briefly, tissue samples from patients with IPF were acquired at the time of lung transplantation. Precision cut lung slices were generated from the explants and cultured for 7 days in the presence of inhibitors (Compound 5, nintedanib, pirfenidone) at clinically relevant concentrations or vehicle (DMSO). Treated slices were lysed in mRNA-compatible buffer for gene expression analysis. For this study, the lysate from n = 6 slices per treatment per lung were pooled for analysis using the nCounter Fibrosis Panel (Nanostring) on the nCounter Max analyzer (Nanostring). This commercially available panel of 770 genes includes genes related to initial tissue damage response, chronic inflammation, proliferation of pro-fibrotic cells, and tissue modification leading to fibrotic disease. Technical quality control and normalization of raw Nanostring mRNA count data was performed using an R framework developed by Bhattacharya et al. (Bhattacharya A. et al., Brief Bioinform. 2021 May 20;22(3):bbaa163). The R packages limma (see URL www.bioconductor.org/packages/release/bioc/html/limma.html) and voom (Law, C.W. et al., Genome Biol. 2014 Feb 3;15(2):R29) were used to detect differentially expressed genes. Benjamini-Hochberg False Discovery Rate (FDR = 5%) was used to adjust p-values for multiple comparisons.

### Results

Individual treatment with either Compound 5, nintedanib, or pirfenidone altered the expression of a subset of genes in the panel (Table C-1 and Table C-2). Overlap was observed in the differentially expressed genes (adj. p<0.05, |log2FC |>0.5) between individual treatments and combinations of Compound 5 + standard of care (FIG. 1, regions B, C, D & F; Tables C-3 through C-6). Several genes, such as COL1A1, COL5A3, and FAP, showed a greater reduction with combination Compound 5 + nintedanib or Compound 5 + pirfenidone treatment than with individual treatment (FIG. 2, Table C-7). A unique set of genes that were not significantly altered by individual treatment were significantly reduced by combination treatment (Tables C-8 through C-10). Some genes, such as TGFB1 and CDH2, were reduced to an extent that was more than additive (Table C-8 and Table C-9). Conclusions

Nintedanib, pirfenidone, and Compound 5 are all therapies targeting fibrosis in IPF. Without wishing to be bound by theory, Compound 5 is believed to work by targeting TGF-beta signaling through inhibition of TGF-beta activation. The mechanism of pirfenidone is not well understood. Nintedanib is known to directly inhibit VEGF, PDGF, and FGF signaling; however, its antifibrotic mechanism in IPF has also been suggested to be through inhibition of TGF-B signaling (see URL www.atsjournals.org/doi/full/10.1165/rcmb.2014-0445OC). Thus, the effect observed in these Examples suggest independent mechanisms of action for these two drugs and that in combination they may provide additional unexpected and surprising benefit to patients with IPF. Indeed, the unique set of fibrosis-related genes altered by each individual treatment suggest that while both nintedanib and pirfenidone have been shown to delay rate of disease progression in patients with IPF, they may do so through mechanisms independent of that predicted for Compound 5. Furthermore, the appearance of a new set of genes only significantly altered with the combination of Compound 5 and either nintedanib or pirfenidone, suggest unexpected synergistic anti-fibrotic effects, which may help explain the positive results observed in Example B15a in patients with IPF.

As noted above, certain genes were upregulated only by a combination of Compound 5 and pirfenidone, which were not upregulated by either Compound 5 alone or pirfenidone alone. Certain genes were downregulated only by a combination of Compound 5 and pirfenidone, which were not downregulated by either Compound 5 alone or pirfenidone alone. Certain genes were upregulated only by a combination of Compound 5 and nintedanib, which were not upregulated by either Compound 5 alone or nintedanib alone. Certain genes were downregulated only by a combination of Compound 5 and nintedanib, which were not downregulated by either Compound 5 alone or pirfenidone alone. Each of these results is surprising and unexpected, as these results are unpredictable based on the gene regulation profile from Compound 5 alone, pirfenidone alone, or nintedanib alone.

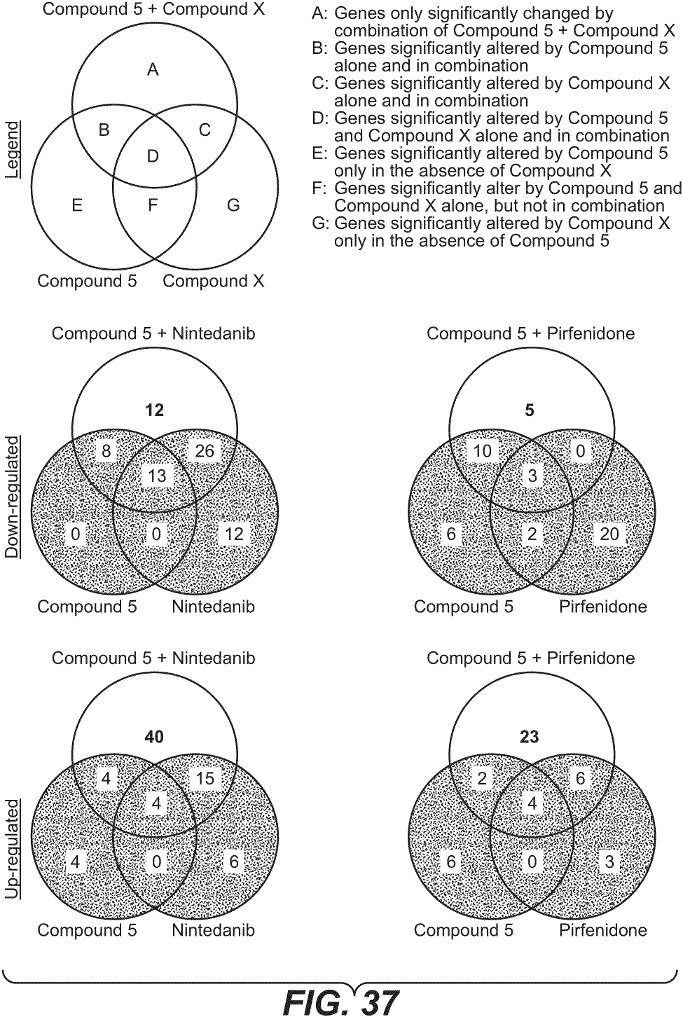

FIG. 37 shows the details of genes that were upregulated or downregulated by Compound 5 alone, by Compound 5 in combination with pirfenidone, and by Compound 5 in combination with nintedanib. Region A of the Venn diagrams shows the number of genes that were upregulated or downregulated by the combination therapy, but not by therapy with Compound 5 alone, pirfenidone alone, or nintedanib alone. Region E shows the number of genes only altered by Compound 5 in the absence of pirfenidone or ninedanib. Region G Docket No.: 76809-20042.00 show the number of genes that were only altered by pirfenidone or nintedanib in the absence of Compound 5. Region F shows the number of genes that were altered by Compound 5 or pirfenidone alone, or by Compound 5 or nintedanib alone, but not by Compound 5 in combination with pirfenidone or by Compound 5 in combination with nintedanib. Again, these results are surprising and are unpredictable based on the gene regulation profile from Compound 5 alone, pirfenidone alone, or nintedanib alone.

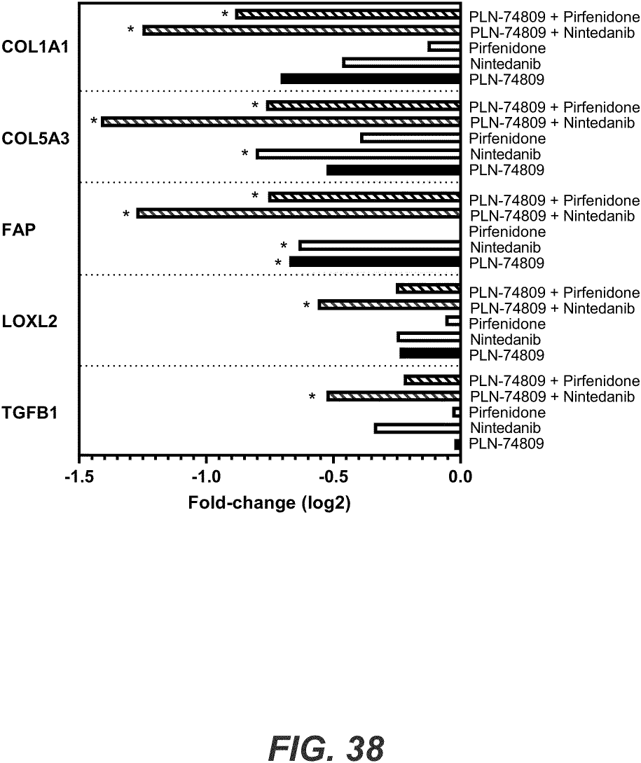

FIG. 38 illustrates the log2 fold-change of a subset of genes that were more greatly reduced by combination of Compound 5 with either nintedanib or pirfenidone (striped bars) than by individual treatments (solid bars), illustrating the unpredictable results of the combinations. Changes that are significant (adj. p<0.05) are noted with an *.

TABLE C-1

Top 20 down-regulated genes (adj. p-value<0.05) for each single treatment

| Compound 5 | | | Nintedanib | | | Pirfenidone | | |
|---|---|---|---|---|---|---|---|---|
| Gene | logFC | adj. p.val | Gene | logFC | adj. p.val | Gene | logFC | adj. p.val |
| COL10A1 | -2.78234 | 0.000151 | FLT1 | -3.36862 | 0.00014 | FGF19 | -1.13967 | 0.00464 |
| POSTN | -0.96042 | 0.015552 | DLL4 | -2.78571 | 7E-05 | KNG1 | -1.09974 | 0.018318 |
| COL5A1 | -0.9282 | 0.007189 | CDH5 | -2.32838 | 4.5E-06 | CDH5 | -1.0568 | 0.012345 |
| MARCO | -0.91829 | 0.012752 | COX4I2 | -2.06987 | 4.5E-06 | MMRN1 | -0.97837 | 0.021315 |
| MMP8 | -0.87544 | 0.016353 | PECAM1 | -1.96726 | 4.5E-06 | PECAM1 | -0.93537 | 0.02647 |
| COL6A3 | -0.83126 | 0.03351 | CETP | -1.73154 | 0.00268 | CXCR4 | -0.88519 | 0.020635 |
| GREM1 | -0.82694 | 0.016353 | CXCR4 | -1.72738 | 4.5E-06 | CD34 | -0.83555 | 0.012207 |
| PECAM1 | -0.82507 | 0.045542 | CXCL10 | -1.70979 | 0.03079 | RELN | -0.82833 | 0.036645 |
| COL1A2 | -0.80618 | 0.002928 | NOTCH4 | -1.60537 | 9.4E-05 | TEK | -0.81239 | 0.012345 |
| CXCR4 | -0.78219 | 0.035154 | CD34 | -1.51584 | 2E-05 | COL14A1 | -0.77855 | 0.012207 |
| COL3A1 | -0.74491 | 0.037012 | FLT4 | -1.47619 | 0.0033 | PDGFRB | -0.75145 | 0.004072 |
| LOX | -0.72205 | 0.029609 | MMP12 | -1.41126 | 0.02574 | COX4I2 | -0.7471 | 0.036724 |
| MMP11 | -0.67488 | 0.016353 | NOS3 | -1.35588 | 0.00246 | GREM1 | -0.74469 | 0.032257 |
| FAP | -0.67354 | 0.004753 | ACVRL1 | -1.32943 | 8.1E-05 | HAVCR1 | -0.73129 | 0.01924 |
| PDGFRB | -0.67322 | 0.004146 | TEK | -1.20595 | 0.00025 | ACVRL1 | -0.72785 | 0.020635 |
| FN1 | -0.66199 | 0.004146 | TPSAB1/B2 | -1.19459 | 9.4E-05 | NOTCH4 | -0.71641 | 0.047965 |
| SERPINE1 | -0.63865 | 0.004146 | COL10A1 | -1.16857 | 0.01678 | COL4A1 | -0.70237 | 0.046538 |
| PLPP4 | -0.63636 | 0.029609 | MMP9 | -1.1396 | 0.01598 | GAS1 | -0.64188 | 0.008677 |
| LOXL1 | -0.62786 | 0.000289 | MMP8 | -1.05279 | 0.00215 | CXCL12 | -0.5918 | 0.035003 |
| TIMP1 | -0.61009 | 0.011136 | MMP11 | -1.00227 | 0.00028 | IFNG | -0.58529 | 0.044415 |

TABLE C-2

Top 20 up-regulated genes (adj. p-value<0.05) for each single treatment

| Compound 5 | | | Nintedanib | | | Pirfenidone | | |
|---|---|---|---|---|---|---|---|---|
| Gene | logFC | adj. p.val | Gene | logFC | adj. p.val | Gene | logFC | adj. p.val |
| CCL13 | 0.94454 | 0.005554 | GPX2 | 1.178309 | 0.002668 | SAA1 | 1.652701 | 0.020635 |
| IFI6 | 0.939098 | 0.002928 | FST | 1.091954 | 4.50E-06 | C6 | 1.475141 | 0.006507 |
| CXCL2 | 0.706869 | 0.016353 | CYP2J2 | 1.025089 | 0.003376 | MMP7 | 1.413219 | 0.012207 |
| MET | 0.670658 | 0.024208 | ADH1C | 0.93374 | 0.000964 | CFTR | 1.189228 | 0.008677 |
| NOS1 | 0.668361 | 0.035154 | ADH1B | 0.897535 | 0.001796 | MET | 1.095964 | 0.001461 |
| APOA2 | 0.613518 | 0.041899 | CFTR | 0.890676 | 0.027441 | PTGS2 | 0.946583 | 0.043689 |
| OAS1 | 0.610373 | 0.016353 | ELN | 0.86887 | 0.000714 | WWC1 | 0.812722 | 0.007601 |
| CIITA | 0.589512 | 0.016353 | MASP1 | 0.816434 | 0.006077 | CXCL2 | 0.77799 | 0.012207 |
| WWC1 | 0.588485 | 0.037012 | KLF5 | 0.812393 | 0.018587 | KLF5 | 0.734315 | 0.046538 |
| TTN | 0.58318 | 0.026329 | CCL19 | 0.788993 | 0.048846 | COL7A1 | 0.670258 | 0.032257 |
| ALDH7A1 | 0.570023 | 0.024773 | WWC1 | 0.751819 | 0.004142 | ALDH7A1 | 0.611935 | 0.01924 |
| CD19 | 0.530067 | 0.044317 | ALDH7A1 | 0.740185 | 0.001817 | OCLN | 0.595599 | 0.010303 |
| LTA | 0.499399 | 0.010559 | MET | 0.713118 | 0.009104 | F11R | 0.580326 | 0.036007 |
| GPC4 | 0.486011 | 0.017161 | LAMA3 | 0.687145 | 0.020833 | LYN | 0.478543 | 0.02647 |
| TNF | 0.48033 | 0.016353 | ACTA2 | 0.684137 | 0.010231 | PSENEN | 0.478411 | 0.017647 |
| XAF1 | 0.452915 | 0.035154 | IGF1 | 0.631994 | 0.042673 | EGFR | 0.476382 | 0.040239 |
| SMAD3 | 0.452828 | 0.004146 | COL6A5 | 0.630915 | 0.035275 | GPC4 | 0.461302 | 0.02647 |
| FZD5 | 0.446597 | 0.027074 | PYGM | 0.623671 | 0.024385 | ACACA | 0.439233 | 0.035608 |
| IFI35 | 0.441218 | 0.042541 | OCLN | 0.602589 | 0.003303 | HADH | 0.437713 | 0.021199 |
| PTGER4 | 0.438995 | 0.033553 | AMOTL2 | 0.576664 | 0.006077 | ICAM1 | 0.432177 | 0.047968 |

TABLE C-3

Corresponding list of genes down-regulated by Compound 5 and/or nintedanib in the different regions of the Venn diagram in FIG. 37 . Headings A-G refer to regions of the Venn diagram explained in FIG. 37 legend
Genes Down-regulated with Compound 5 and Nintedanib Alone or in Combination

| | | | | | | |
|---|---|---|---|---|---|---|
| A | B | C | D | E | F | G |
| APOC2 | ANGPTL4 | ACVRL1 | COL10A1 | | | CD14 |
| CDH2 | COL1A2 | CD34 | COL6A3 | | | CD209 |
| COL1A1 | COL3A1 | CDH5 | CXCR4 | | | CTSB |
| COL4A2 | COL5A1 | CETP | FAP | | | CXCL10 |
| FCGR3A/B | FN1 | COL4A1 | GREM1 | | | CYBB |
| ITGB3 | LOXL1 | COL5A3 | LOX | | | FCER1A |
| LOXL2 | MARCO | COX4I2 | MMP11 | | | IL10 |
| NID1 | SERPINE1 | CPA3 | MMP8 | | | LILRB2 |
| SERPINH1 | | DLL4 | PDGFRB | | | MMP12 |
| SPP1 | | FLI1 | PECAM1 | | | MMP9 |
| TGFB1 | | FLT1 | PLPP4 | | | MS4A4A |
| THBS2 | | FLT4 | POSTN | | | PREX1 |
| | | ITGA5 | TIMP1 | | | |
| | | KDR | | | | |
| | | MMP1 | | | | |
| | | MMP14 | | | | |
| | | MMP16 | | | | |
| | | MMP2 | | | | |
| | | MMRN1 | | | | |
| | | MS4A2 | | | | |
| | | NID2 | | | | |
| | | NOS3 | | | | |
| | | NOTCH4 | | | | |
| | | PDGFB | | | | |
| | | TEK | | | | |
| | | TPSAB1/B2 | | | | |

TABLE C-4

Corresponding list of genes up-regulated by Compound 5 and/or nintedanib in the different regions of the Venn diagram in FIG. 37 . Headings A-G refer to regions of the Venn diagram explained in FIG. 37 legend
Genes up-regulated with Compound 5 and Nintedanib alone or in combination

| | | | | | | |
|---|---|---|---|---|---|---|
| A | B | C | D | E | F | G |
| ACACA | CD19 | ADH1B | ALDH7A1 | APOA2 | | ACTA2 |
| AKR1B10 | NOS1 | ADH1C | CXCL2 | CCL13 | | CCL19 |
| APOB | OAS1 | ALDH3A2 | MET | CIITA | | COL6A5 |
| BCL2L1 | TTN | AMOTL2 | WWC1 | IFI6 | | ELN |
| C3 | | CFTR | | | | IGF1 |
| C6 | | CYP2J2 | | | | PYGM |
| CCL2 | | FST | | | | |
| CXCL8 | | GPX2 | | | | |
| CYP4A11/22 | | HCAR2 | | | | |
| DAPK1 | | HKDC1 | | | | |
| DLL1 | | KLF5 | | | | |
| EGFR | | LAMA3 | | | | |
| ELOVL6 | | MAPK10 | | | | |
| EPHX2 | | MASP1 | | | | |
| F11R | | OCLN | | | | |
| FASN | | | | | | |
| FLNB | | | | | | |
| FZD5 | | | | | | |
| GCNT1 | | | | | | |
| GPC4 | | | | | | |
| HADH | | | | | | |
| IL1RAP | | | | | | |
| IL20RB | | | | | | |
| JAG2 | | | | | | |
| KIR2DL3 | | | | | | |
| KLRB1 | | | | | | |
| LYN | | | | | | |
| MS4A1 | | | | | | |
| MUC5B | | | | | | |
| PLIN4 | | | | | | |
| PPARGC1A | | | | | | |
| PTGER4 | | | | | | |
| SAA1 | | | | | | |
| SCD | | | | | | |
| SCIN | | | | | | |
| SLC25A10 | | | | | | |
| SLC2A2 | | | | | | |
| SPIB | | | | | | |
| SREBF1 | | | | | | |
| VAMP8 | | | | | | |

TABLE C-5

Corresponding list of genes down-regulated by Compound 5 and/or pirfenidone in the different regions of the Venn diagram in FIG. 37 . Headings A-G refer to regions of the Venn diagram explained in FIG. 37 legend
Genes Down-regulated with Compound 5 and Pirfenidone Alone or in Combination

| | | | | | | |
|---|---|---|---|---|---|---|
| A | B | C | D | E | F | G |
| CDH2 | ANGPTL4 | | COL1A2 | COL3A1 | CXCR4 | ACVRL1 |
| COL1A1 | COL10A1 | | GREM1 | COL6A3 | PECAM1 | CD34 |
| COL5A3 | COL5A1 | | PDGFRB | LOXL1 | | CDH5 |
| ITGAS | FAP | | | MARCO | | COL14A1 |
| THBS2 | FN1 | | | MMP8 | | COL4A1 |
| | LOX | | | PLPP4 | | COX4I2 |
| | MMP11 | | | | | CXCL12 |
| | POSTN | | | | | FGF19 |
| | SERPINE1 | | | | | FLI1 |
| | TIMP1 | | | | | GAS1 |
| | | | | | | HAVCR1 |
| | | | | | | HMGCS2 |
| | | | | | | IFNG |
| | | | | | | KNG1 |
| | | | | | | MMP16 |
| | | | | | | MMRN1 |
| | | | | | | MS4A2 |
| | | | | | | NOTCH4 |
| | | | | | | RELN |
| | | | | | | TEK |

TABLE C-6

Corresponding list of genes up-regulated by Compound 5 and/or pirfenidone in the different regions of the Venn diagram in FIG. 37 . Headings A-G refer to regions of the Venn diagram explained in FIG. 37 legend
Genes Up-regulated with Compound 5 and Pirfenidone Alone or in Combination

| | | | | | | |
|---|---|---|---|---|---|---|
| A | B | C | D | E | F | G |
| BCL2L1 | CCL13 | C6 | ALDH7A1 | APOA2 | | COL7A1 |
| C3 | CD19 | CFTR | CXCL2 | CIITA | | MMP7 |
| CCL4 | | F11R | MET | IFI6 | | PTGS2 |
| CD209 | | KLF5 | WWC1 | NOS1 | | |
| CYP2J2 | | OCLN | | OAS1 | | |
| EGFR | | SAA1 | | TTN | | |
| FLNB | | | | | | |
| GPC4 | | | | | | |
| GZMA | | | | | | |
| HCAR2 | | | | | | |
| HDC | | | | | | |
| IL1B | | | | | | |
| JAG2 | | | | | | |
| LYN | | | | | | |
| MAPK10 | | | | | | |
| MMP12 | | | | | | |
| MUC5B | | | | | | |
| SLC25A10 | | | | | | |
| SPIB | | | | | | |
| SREBF1 | | | | | | |
| TJP2 | | | | | | |
| TNF | | | | | | |
| VAMP8 | | | | | | |

TABLE C-7

Example of profibrogenic genes more greatly reduced by combination of Compound 5 + nintedanib than individual treatment

| | Combination | | Compound 5 | | Nintedanib | |
|---|---|---|---|---|---|---|
| Gene | % Reduction | adj. p.val | % Reduction | adj. p.val | % Reduction | adj. p.val |
| FAP | 58.70 | 8.79E-08 | 37.30 | 0.004753 | 35.72 | 0.002668 |
| LOX | 49.73 | 0.000652 | 39.38 | 0.029609 | 40.76 | 0.014642 |
| PDGFRB | 50.62 | 3.38E-06 | 37.29 | 0.004146 | 42.09 | 0.000251 |
| POSTN | 69.50 | 2.90E-06 | 48.61 | 0.015552 | 38.94 | 0.042673 |
| SERPINE1 | 55.41 | 1.18E-07 | 35.77 | 0.004146 | 9.75 | 0.474747 |

TABLE C-8

Genes only significantly reduced (adj. p<0.05, | log2FC | >0.5) by combination Compound 5 + nintedanib
Genes Significantly Down-regulated Only in Combination Compound 5 + Nintedanib

| | Combination | | Compound 5 | | Nintedanib | |
|---|---|---|---|---|---|---|
| Gene | % Reduction | adj. p.val | % Reduction | adj. p.val | % Reduction | adj. p.val |
| APOC2 | 30.33 | 0.026002112 | -1.20 | 0.951972401 | 16.88 | 0.301062 |
| CDH2 | 30.44 | 0.004175441 | 23.87 | 0.059638037 | -9.99 | 0.47474651 |
| COL1A1 | 57.99 | 0.001270246 | 38.78 | 0.141616566 | 27.60 | 0.28119945 |
| COL4A2 | 51.79 | 0.000387615 | 17.74 | 0.434260859 | 31.55 | 0.08299324 |
| FCGR3A/B | 62.40 | 0.000807898 | 28.54 | 0.253476522 | 33.44 | 0.13945479 |
| ITGB3 | 32.08 | 0.006980062 | -2.48 | 0.885116905 | 16.60 | 0.23580918 |
| LOXL2 | 32.24 | 0.004446976 | 15.32 | 0.295367889 | 15.99 | 0.23580918 |
| NID1 | 32.43 | 0.023985634 | 19.25 | 0.331400207 | 26.59 | 0.11687588 |
| SERPINH1 | 31.23 | 0.001400691 | 22.68 | 0.062155331 | 15.02 | 0.20439659 |
| SPP1 | 57.84 | 0.032418457 | -15.73 | 0.766690315 | 59.73 | 0.05193504 |
| TGFB1 | 30.62 | 0.024213414 | 1.68 | 0.93937587 | 21.04 | 0.19783273 |
| THBS2 | 43.71 | 0.004321846 | 31.47 | 0.136196388 | 25.58 | 0.18794543 |

TABLE C-9

Genes only significantly reduced (adj. p<0.05, | log2FC | >0.5) by combination Compound 5 + pirfenidone
Genes Significantly Down-regulated Only in Combination Compound 5 + Pirfenidone

| | Combination | | Compound 5 | | Pirfenidone | |
|---|---|---|---|---|---|---|
| Gene | % Reduction | adj. p.val | % Reduction | adj. p.val | % Reduction | adj. p.val |
| CDH2 | 34.08 | 0.003167904 | 23.87 | 0.059638037 | -7.12 | 0.654318 |
| COL1A1 | 45.92 | 0.037471567 | 38.78 | 0.141616566 | 8.58 | 0.81440377 |
| COL5A3 | 41.17 | 0.038815444 | 30.59 | 0.204926751 | 23.95 | 0.30307956 |
| ITGAS | 29.94 | 0.000642442 | 26.20 | 0.004145647 | 26.21 | 0.00650743 |
| THBS2 | 40.85 | 0.019109184 | 31.47 | 0.136196388 | 21.20 | 0.32129762 |

TABLE C-10

Lists of all genes that are only significantly altered (adj. p<0.05, | log2FC | >0.5) by combined treatment with Compound 5 and either nintedanib or pirfenidone (region A in the Venn diagrams of FIG. 37 )

| Down-regulated | | Up-regulated | |
|---|---|---|---|
| Compound 5 + Nintedanib | Compound 5 + Pirfenidone | Compound 5 + Nintedanib | Compound 5 + Pirfenidone |
| APOC2 | CDH2 | ACACA | BCL2L1 |
| CDH2 | COL1A1 | AKR1B10 | C3 |
| COL1A1 | COL5A3 | APOB | CCL4 |
| COL4A2 | ITGAS | BCL2L1 | CD209 |
| FCGR3A/B | THBS2 | C3 | CYP2J2 |
| ITGB3 | | C6 | EGFR |
| LOXL2 | | CCL2 | FLNB |
| NID1 | | CXCL8 | GPC4 |
| SERPINH1 | | CYP4A11/22 | GZMA |
| SPP1 | | DAPK1 | HCAR2 |
| TGFB1 | | DLL1 | HDC |
| THBS2 | | EGFR | IL1B |
| | | ELOVL6 | JAG2 |
| | | EPHX2 | LYN |
| | | F11R | MAPK10 |
| | | FASN | MMP12 |
| | | FLNB | MUC5B |
| | | FZD5 | SLC25A10 |
| | | GCNT1 | SPIB |
| | | GPC4 | SREBF1 |
| | | HADH | TJP2 |
| | | IL1RAP | TNF |
| | | IL20RB | VAMP8 |
| | | JAG2 | |
| | | KIR2DL3 | |
| | | KLRB1 | |
| | | LYN | |
| | | MS4A1 | |
| | | MUC5B | |
| | | PLIN4 | |
| | | PPARGC1A | |
| | | PTGER4 | |
| | | SAA1 | |
| | | SCD | |
| | | SCIN | |
| | | SLC25A10 | |
| | | SLC2A2 | |
| | | SPIB | |
| | | SREBF1 | |
| | | VAMP8 | |

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method of treating a subject for a disease, comprising:
administering to the subject a first drug comprising a compound of formula (II): 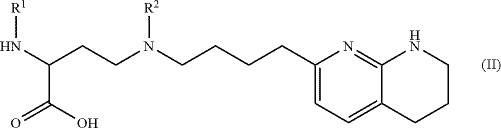
or a salt thereof; and
administering to the subject at least a second drug that is selected from the group consisting of: pirfenidone and nintedanib, or a salt thereof, whereby the subject is treated for the disease;
wherein in the compound of Formula (II):
R¹ is C₆-C₁₄ aryl or 5- to 10-membered heteroaryl wherein the C₆-C₁₄ aryl and 5- to 10-membered heteroaryl are optionally substituted by R^{la};
R² is hydrogen; deuterium; C₁-C₆ alkyl optionally substituted by R^{2a}; —OH; —O—C₁-C₆ alkyl optionally substituted by R^{2a}; C₃-C₆ cycloalkyl optionally substituted by R^{2b}; —O—C₃-C₆ cycloalkyl optionally substituted by R^{2b}; 3- to 12-membered heterocyclyl optionally substituted by R^{2c}; or -S(O)₂R^{2d}; with the proviso that any carbon atom bonded directly to a nitrogen atom is optionally substituted with an R^{2a} moiety other than halogen;
each R^{1a} is independently C₁-C₆ alkyl, C₂-C₆ alkenyl, C₂-C₆ alkynyl, C₃-C₈ cycloalkyl, C₄-C₈ cycloalkenyl, 3- to 12-membered heterocyclyl, 5- to 10-membered heteroaryl, C₆-C₁₄ aryl, deuterium, halogen, —CN, -OR³, -SR³, -NR⁴R⁵, —NO₂, -C=NH(OR³), -C(O)R³, -OC(O)R³, -C(O)OR³, -C(O)NR⁴R⁵, -NR³C(O)R⁴, -NR³C(O)OR⁴, -NR³C(O)NR⁴R⁵, -S(O)R³, —S(O)₂R³, -NR³S(O)R⁴, -NR³S(O)₂R⁴, -S(O)NR⁴R⁵, -S(O)₂NR⁴R⁵, or -P(O)(OR⁴)(OR⁵), wherein each R^{1a} is, where possible, independently optionally substituted by deuterium, halogen, oxo, —OR⁶, -NR⁶R⁷, -C(O)R⁶, —CN, -S(O)R⁶, -S(O)₂R⁶, -P(O)(OR⁶)(OR⁷), C₃-C₈ cycloalkyl, 3- to 12-membered heterocyclyl, 5- to 10-membered heteroaryl, C₆-C₁₄ aryl, or C₁-C₆ alkyl optionally substituted by deuterium, oxo, —OH or halogen;
each R^{2a}, R^{2b}, R^{2c}, R^{2e}, and R^{2f} is independently oxo or R^{1a};
R^{2d} is C₁-C₆ alkyl optionally substituted by R^{2e} or C₃-C₅ cycloalkyl optionally substituted by R^{2f};
R³ is independently hydrogen, deuterium, C₁-C₆ alkyl, C₂-C₆ alkenyl, C₂-C₆ alkynyl, C₃-C₆ cycloalkyl, C₆-C₁₄ aryl, 5- to 10-membered heteroaryl or 3- to 12-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 10-membered heteroaryl and 3- to 12-membered heterocyclyl of R³ are independently optionally substituted by halogen, deuterium, oxo, —CN, -OR⁸, -NR⁸R⁹, -P(O)(OR⁸)(OR⁹), or C₁-C₆ alkyl optionally substituted by deuterium, halogen, —OH or oxo;
R⁴ and R⁵ are each independently hydrogen, deuterium, C₁-C₆ alkyl, C₂-C₆ alkenyl, C₂-C₆ alkynyl, C₃-C₆ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₆ cycloalkyl, C₆-C₁₄ aryl, 5- to 6-membered heteroaryl and 3- to 6-membered heterocyclyl of R⁴ and R⁵ are independently optionally substituted by deuterium, halogen, oxo, —CN, -OR⁸, -NR⁸R⁹ or C₁-C₆ alkyl optionally substituted by deuterium, halogen, —OH or oxo;
or R⁴ and R⁵ are taken together with the atom to which they attached to form a 3- to 6-membered heterocyclyl optionally substituted by deuterium, halogen, oxo, -OR⁸, -NR⁸R⁹ or C₁-C₆ alkyl optionally substituted by deuterium, halogen, oxo or —OH;
R⁶ and R⁷ are each independently hydrogen, deuterium, C₁-C₆ alkyl optionally substituted by deuterium, halogen, or oxo, C₂-C₆ alkenyl optionally substituted by deuterium, halogen, or oxo, or C₂-C₆ alkynyl optionally substituted by deuterium, halogen, or oxo;
or R⁶ and R⁷ are taken together with the atom to which they attached to form a 3- to 6-membered heterocyclyl optionally substituted by deuterium, halogen, oxo or C₁-C₆ alkyl optionally substituted by deuterium, halogen, or oxo; and
R⁸ and R⁹ are each independently hydrogen, deuterium, C₁-C₆ alkyl optionally substituted by deuterium, halogen, or oxo, C₂-C₆ alkenyl optionally substituted by deuterium, halogen or oxo, or C₂-C₆ alkynyl optionally substituted by deuterium, halogen, or oxo;
or R⁸ and R⁹ are taken together with the atom to which they attached to form a 3-6 membered heterocyclyl optionally substituted by deuterium, halogen, oxo or C₁-C₆ alkyl optionally substituted by deuterium, oxo, or halogen.

2. The method of claim 1, wherein in the compound of Formula (II) or a salt thereof, R¹ is 5- to 10-membered heteroaryl optionally substituted by R^{1a}.

3. The method of claim 1, wherein in the
compound of Formula (II), or a salt thereof, R¹ is:
pyrimidinyl, quinazolinyl, pyrazolopyrimidinyl, pyrazinyl, quinolinyl, pyridopyrimidinyl, thienopyrimidinyl, pyridinyl, pyrrolopyrimidinyl, quinoxalinyl, indazolyl, benzothiazolyl, naphthalenyl, purinyl, or isoquinolinyl; and
optionally substituted by deuterium, hydroxy, C₁-C₆ alkyl, C₁-C₆ haloalkyl, C₁-C₆ perhaloalkyl, C₁-C₆ alkoxyl, C₃-C₈ cycloalkyl, C₃-C₈ halocycloalkyl, C₃-C₈ cycloalkoxyl, 5- to 10-membered heteroaryl, C₆-C₁₄ aryl, cyano, amino, alkylamino, or dialkylamino.

4. The method of claim 1,
wherein in the compound of Formula (II), or a salt thereof, R¹ is:
pyrimidin-2-yl, pyrimidin-4-yl, quinazolin-4-yl, 1H-pyrazolo[3,4-d]pyrimidine-4-yl, 1H-pyrazolo[4,3-d]pyrimidine-7-yl, pyrazin-2-yl, quinoline-4-yl, pyrido[2,3-d]pyrimidin-4-yl, pyrido[3,2-d]pyrimidin-4-yl, pyrido[3,4-d]pyrimidin-4-yl, thieno[2,3-d]pyrimidin-4-yl, thieno[3,2-d]pyrimidin-4-yl, thienopyrimidin-4-yl, pyridin-2-yl, pyridin-3-yl, 7H-pyrrolo[2,3-d]pyrimidin-4-yl, quinoxalin-2-yl, 1H-indazol-3-yl, benzo[d]thiazol-2-yl, naphthalen-1-yl, 9H-purin-6-yl, or isoquinolin-1-yl; and
optionally substituted by: one or more deuterium; methyl; cyclopropyl; fluoro; chloro; bromo; difluoromethyl; trifluoromethyl; methyl and fluoro; methyl and trifluoromethyl; methoxy; cyano; dimethylamino; phenyl; pyridin-3-yl; or pyridin-4-yl.

5. The method of claim 1, wherein in the compound of Formula (II), or a salt thereof, R¹ is pyrimidin-4-yl optionally substituted by R^{1a} ; or
wherein in the compound of Formula (II), or a salt thereof, R¹ is pyrimidin-4-yl optionally substituted by R^{1a} wherein R^{1a} is 5- to 10-membered heteroaryl or C₁-C₆ alkyl optionally substituted by halogen; or
wherein in the compound of Formula (II), or a salt thereof, R¹ is pyrimidin-4-yl optionally substituted by pyrazolyl, methyl, difluoromethyl, or trifluoromethyl; or
wherein in the compound of Formula (II), or a salt thereof, R¹ is pyrimidin-4-yl substituted by both methyl and trifluoromethyl.

6. The method of claim 1, wherein in the compound of Formula (II), or a salt thereof, R¹ is quinazolin-4-yl optionally substituted by R^{1a} ; or
wherein in the compound of Formula (II), or a salt thereof, R¹ is quinazolin-4-yl optionally substituted by halogen, C₁-C₆ alkyl optionally substituted by halogen, or C₁-C₆ alkoxy; or
wherein in the compound of Formula (II), or a salt thereof, R¹ is quinazolin-4-yl optionally substituted by fluoro, chloro, methyl, trifluoromethyl or methoxy.

7. The method of claim 1, wherein in the compound of Formula (II), or a salt thereof, R² is:
hydrogen;
deuterium;
hydroxy; or
C₁-C₆ alkyl or C₁-C₆ alkoxyl optionally substituted with: deuterium, halogen, C₁-C₆ alkyl, C₁-C₆ haloalkyl, C₁-C₆ hydroxyalkyl, C₁-C₆ alkoxyl, C₃-C₈ cycloalkyl, C₃-C₈ halocycloalkyl, C₃-C₈ cycloalkoxyl, C₆-C₁₄ aryl, C₆-C₁₄ aryloxy, 5- to 10-membered heteroaryl, 5- to 10-membered heteroaryloxy, 3- to 12-membered heterocyclyl optionally substituted with oxo, -C(O)NR⁴R⁵, -NR³C(O)R⁴, or -S(O)₂R³ ; or
wherein in the compound of Formula (II), or a salt thereof, R² is:
methyl, methoxy, ethyl, ethoxy, propyl, cyclopropyl, or cyclobutyl;
each of which is optionally substituted with one or more of: hydroxy, methoxy, ethoxy, acetamide, fluoro, fluoroalkyl, phenoxy, dimethylamide, methylsulfonyl, cyclopropoxyl, pyridin-2-yloxy, optionally methylated or fluorinated pyridine-3-yloxy, N-morpholinyl, N-pyrrolidin-2-onyl, dimethylpyrazol-1-yl, dioxiran-2-yl, morpholin-2-yl, oxetan-3-yl, phenyl, tetrahydrofuran-2-yl, thiazol-2-yl;
each of which is substituted with 0, 1, 2, or 3 of deuterium, hydroxy, methyl, fluoro, cyano, or oxo; or
wherein in the compound of Formula (II), or a salt thereof, R² is C₁-C₆ alkyl optionally substituted by R^{2a} ; or
wherein in the compound of Formula (II), or a salt thereof, R² is C₁-C₆ alkyl optionally substituted by R^{2a} wherein R^{2a} is: halogen; C₃-C₈ cycloalkyl optionally substituted by halogen; 5-to 10-membered heteroaryl optionally substituted by C₁-C₆ alkyl; -NR⁴R⁵; -NR³C(O)R⁴; -S(O)₂R³; or oxo; or
wherein in the compound of Formula (II), or a salt thereof, R² is C₁-C₆ alkyl optionally substituted by R^{2a} wherein R^{2a} is: fluoro; cyclobutyl substituted by fluoro; pyrazolyl substituted by methyl; or —S(O)₂CH₃ ; or
wherein in the compound of Formula (II), or a salt thereof, R² is C₁-C₆ alkyl optionally substituted by -OR³ ; or
wherein in the compound of Formula (II), or a salt thereof, R² is C₁-C₆ alkyl optionally substituted by -OR³, and R³ is: hydrogen; C₁-C₆ alkyl optionally substituted by halogen; C₃-C₆ cycloalkyl optionally substituted by halogen; C₆-C₁₄ aryl optionally substituted by halogen; or 5-to 6-membered heteroaryl optionally substituted by halogen or C₁-C₆ alkyl; or
wherein in the compound of Formula (II), or a salt thereof, R² is C₁-C₆ alkyl optionally substituted by -OR³, and R³ is: hydrogen; methyl; ethyl; difluoromethyl; -CH₂CHF₂; —CH₂CF₃; cyclopropyl substituted by fluoro; phenyl optionally substituted by fluoro; or pyridinyl optionally substituted by fluoro or methyl.

8. The method of claim 1, wherein in the compound of Formula (II), or a salt thereof, R² is —CH₂CH₂OCH₃.

9. The method of claim 1, wherein in the compound of Formula (II), or a salt thereof, R² is C₁-C₆ alkyl substituted by both halogen and OR³, wherein R³ is C₁-C₆ alkyl; or
wherein in the compound of Formula (II), or a salt thereof, R² is C₃-C₆ cycloalkyl optionally substituted by R^{2b}; or
wherein in the compound of Formula (II), or a salt thereof, R² is cyclopropyl.

10. The method of claim 1, wherein in the compound of Formula (II), or a salt thereof, R¹ is 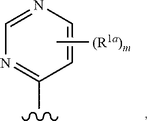
wherein m is 0, 1, 2, or 3 and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or
wherein in the compound of Formula (II), or a salt thereof, R¹ is 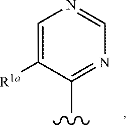 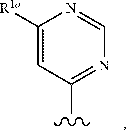 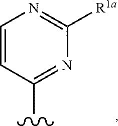 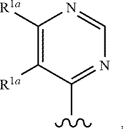 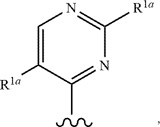 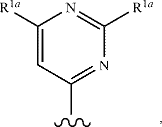 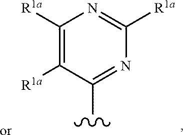
wherein each R
^{1a} is independently deuterium, alkyl, haloalkyl, or heteroaryl; or
wherein in the compound of Formula (II), or a salt thereof, R¹ is 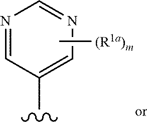 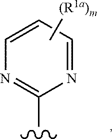
wherein m is 0, 1, 2, or 3 and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or
wherein in the compound of Formula (II), or a salt thereof, R¹ is 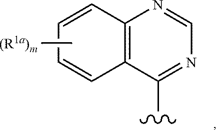
wherein m is 0, 1, 2, 3, 4, or 5 and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or
wherein in the compound of Formula (II), or a salt thereof, R¹ is 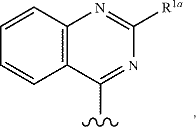 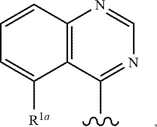 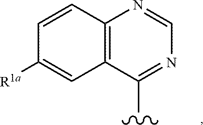 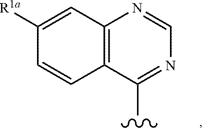 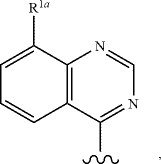 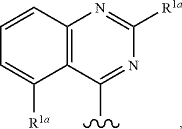 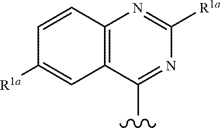 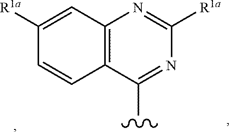 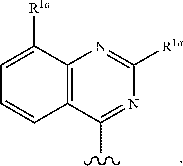 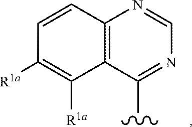 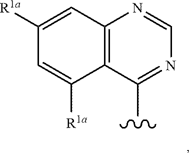 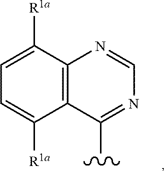 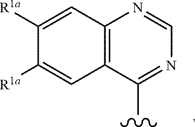 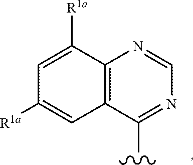 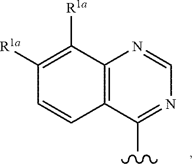 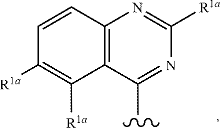 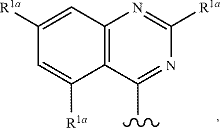 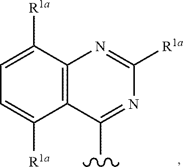 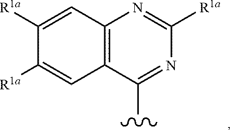 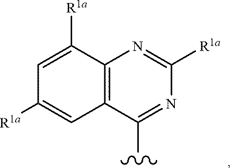 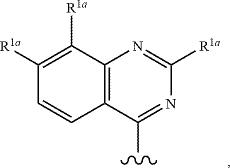 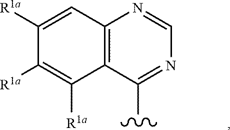 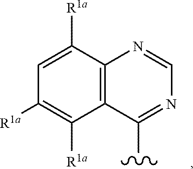 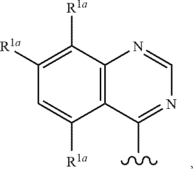 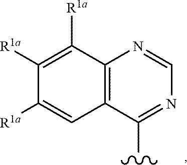 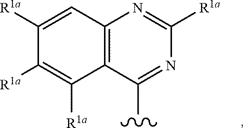 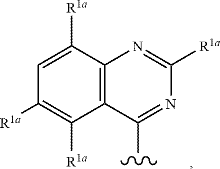 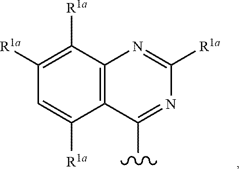 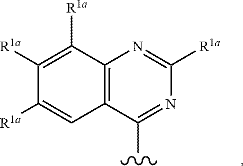 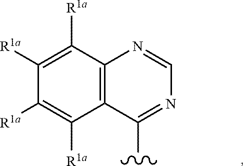 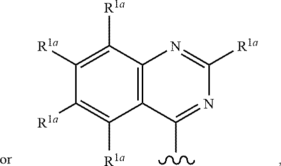
wherein each R
^{1a} is independently deuterium, halogen, alkyl, haloalkyl, or alkoxy; or.
wherein in the compound of Formula (II), or a salt thereof, R¹ is 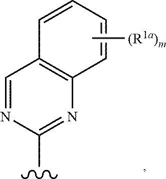 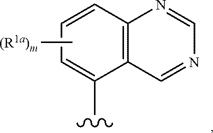 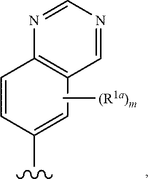 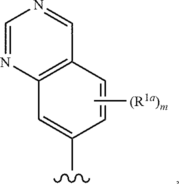 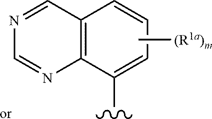
wherein m is 0, 1, 2, 3, 4, or 5 and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or
wherein in the compound of Formula (II), or a salt thereof, R¹ is 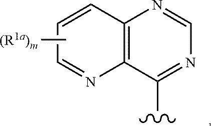
wherein m is 0, 1, 2, 3, or 4, and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or.
wherein in the compound of Formula (II), or a salt thereof, R¹ is selected from the group consisting of 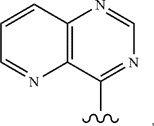 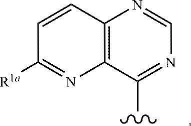 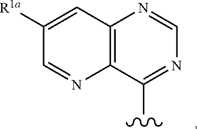 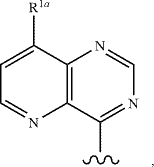 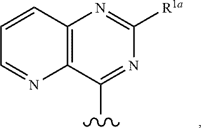 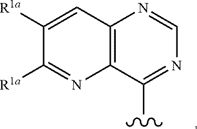 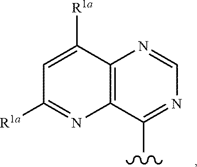 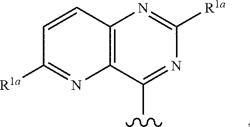 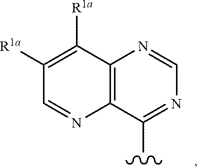 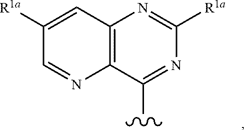 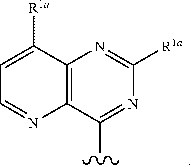 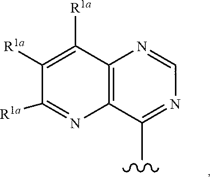 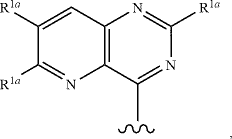 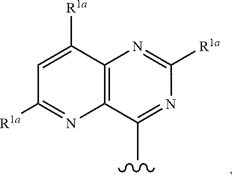 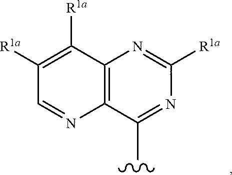 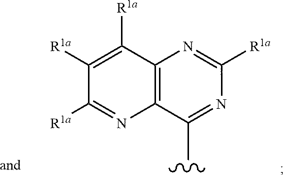 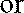
wherein in the compound of Formula (II), or a salt thereof, R¹ is 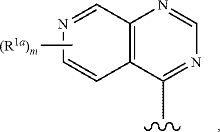
wherein m is 0, 1, 2, 3, or 4, and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or
wherein in the compound of Formula (II), or a salt thereof, R¹ is selected from the group consisting of 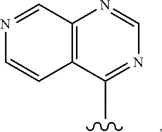 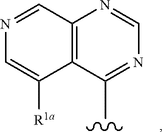 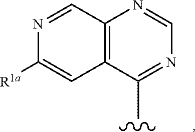 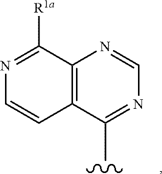 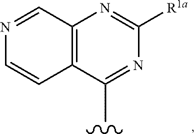 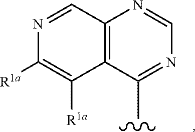 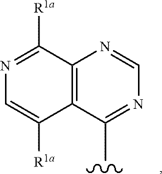 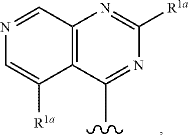 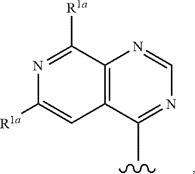 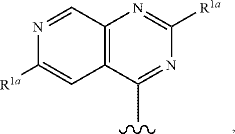 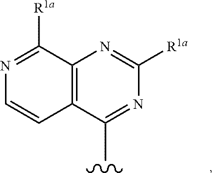 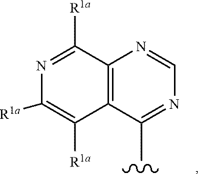 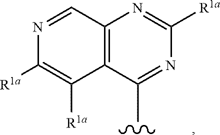 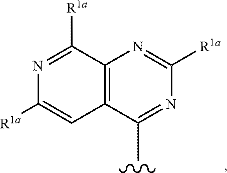 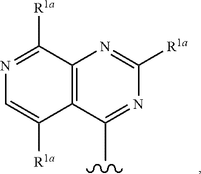 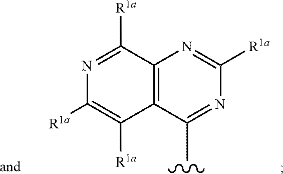
or
wherein in the compound of Formula (II), or a salt thereof, R¹ is 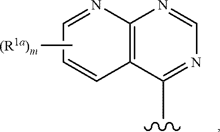
wherein m is 0, 1, 2, 3, or 4, and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or wherein in the compound of Formula (II), or a salt thereof, R¹ is selected from the group consisting of 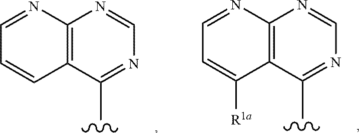 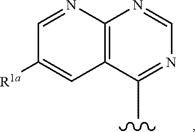 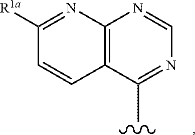 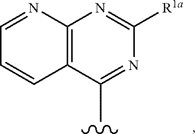 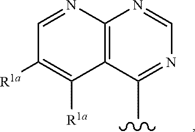 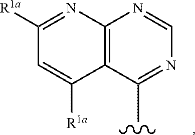 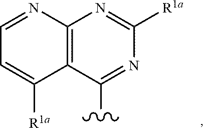 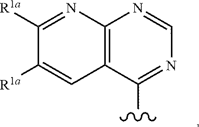 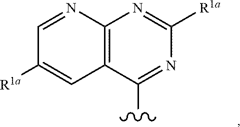 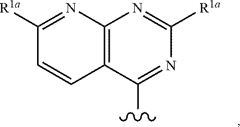 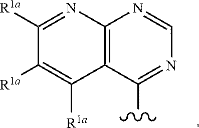 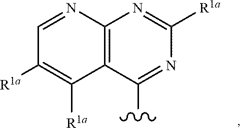 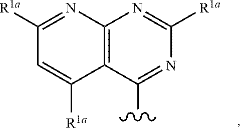 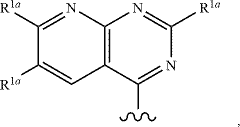 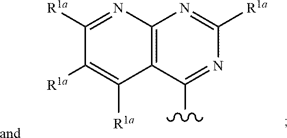
or
wherein in the compound of Formula (II), or a salt thereof, R¹ is 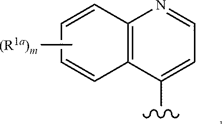
wherein m is 0, 1, 2, 3, 4, 5, or 6 and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or
wherein in the compound of Formula (II), or a salt thereof, R¹ is 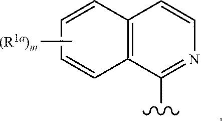
wherein m is 0, 1, 2, 3, 4, 5, or 6 and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or
wherein in the compound of Formula (II), or a salt thereof, R¹ is 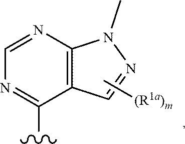
wherein m is 0, 1, or 2 and each R
^{1a} is, where applicable, independently deuterium, halogen, alkyl, haloalkyl, alkoxy, hydroxy, —CN, or heteroaryl, wherein the alkyl, haloalkyl, alkoxy, hydroxy, and heteroaryl of R^{1a} are independently optionally substituted by deuterium; or
wherein in the compound of Formula (II), or a salt thereof, R¹ is selected from the group consisting of 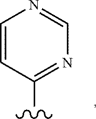 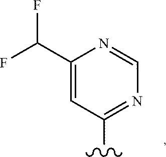 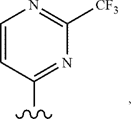 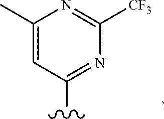 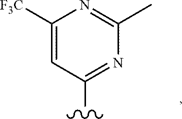 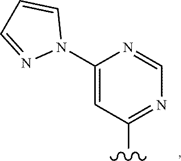 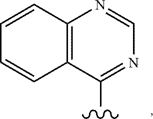 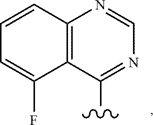 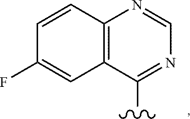 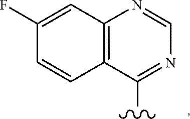 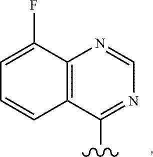 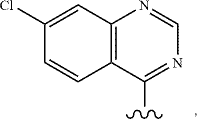 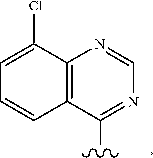 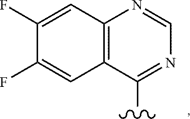 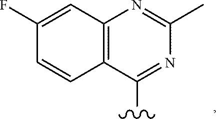 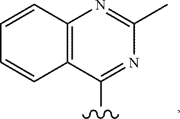 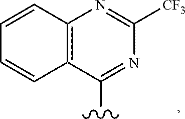 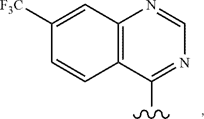 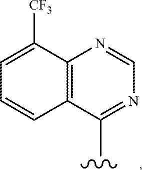 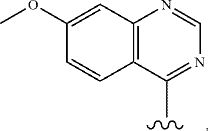 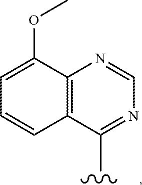 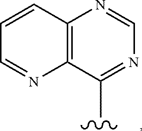 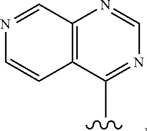 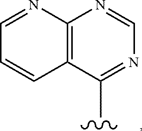 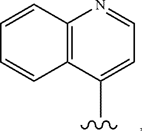 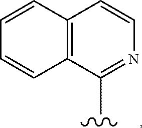 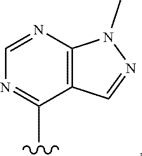
and any of the foregoing groups wherein any one or more hydrogen atom(s) are replaced with deuterium atom(s); or
wherein in the compound of Formula (II), or a salt thereof, R¹ is selected from the group consisting of 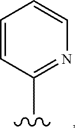 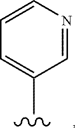 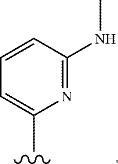 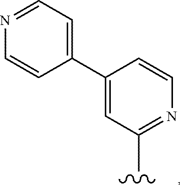 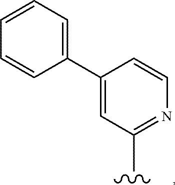 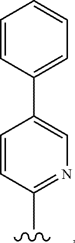 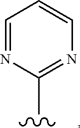 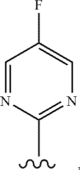 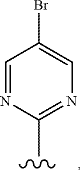 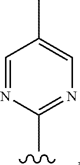 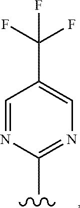 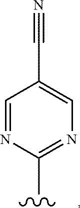 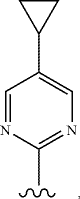 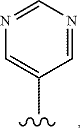 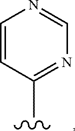 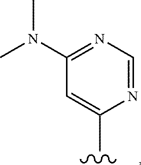 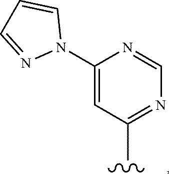 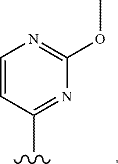 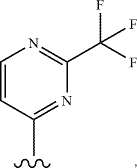 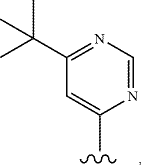 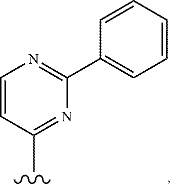 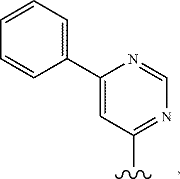 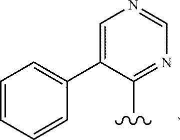 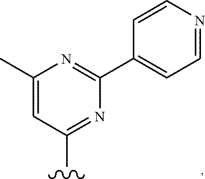 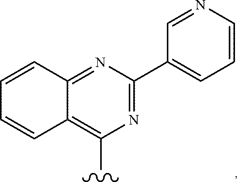 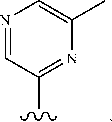 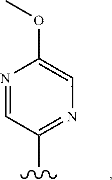 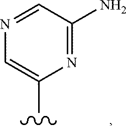 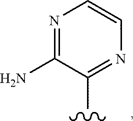 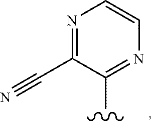 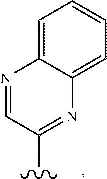 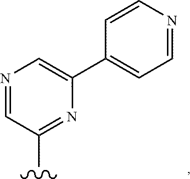 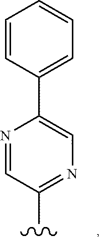 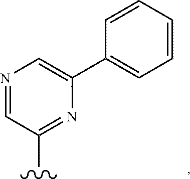 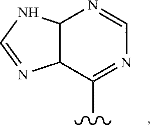 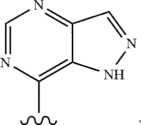 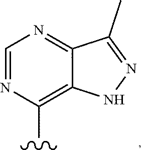 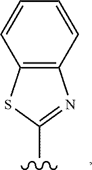 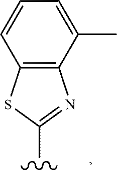 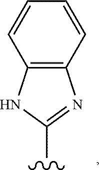 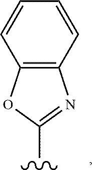 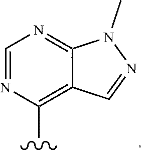 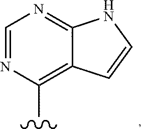 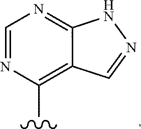 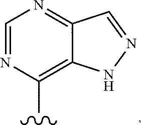 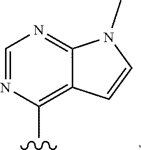 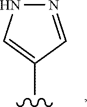 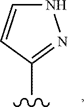
and any of the foregoing groups wherein any one or more hydrogen atom(s) are replaced with deuterium atom(s); or
wherein in the compound of Formula (II), or a salt thereof, R¹ is selected from the group consisting of 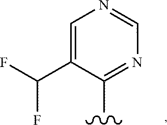 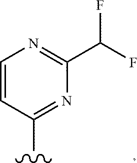 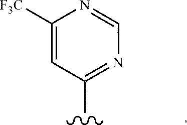 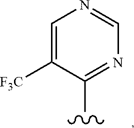 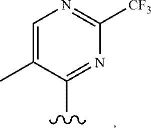 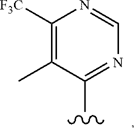 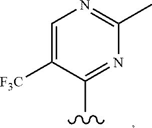 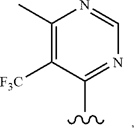 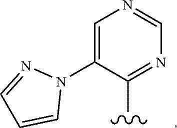 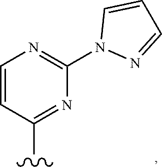 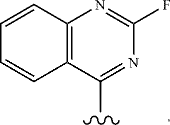 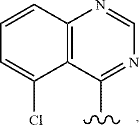 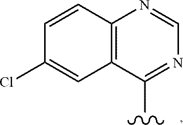 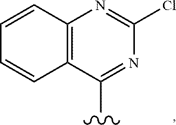 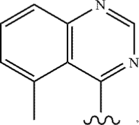 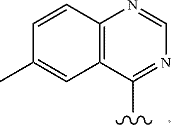 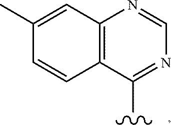 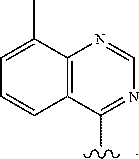 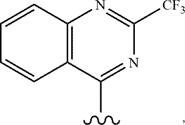 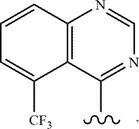 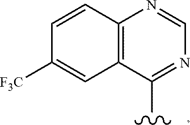 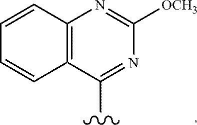 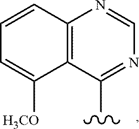 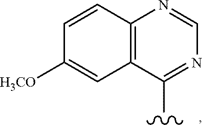 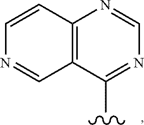 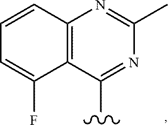 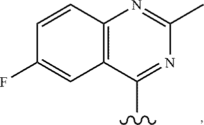 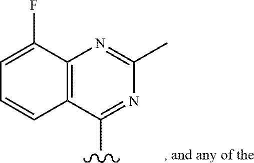
foregoing groups wherein any one or more hydrogen atom(s) are replaced with deuterium atom(s); or
wherein in the compound of Formula (II), or a salt thereof, R¹ is selected from the group consisting of 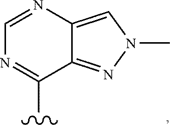 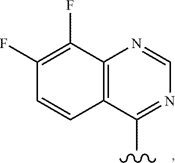 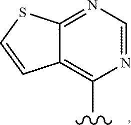 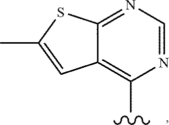 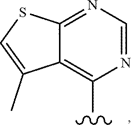 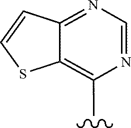 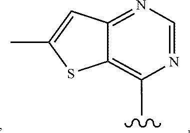 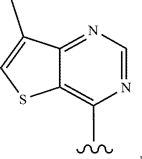 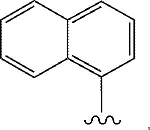
and any of the foregoing groups wherein any one or more hydrogen atom(s) are replaced with deuterium atom(s).

11. The method of claim 1, wherein in the compound of Formula (II), or a salt thereof, R² is 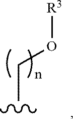
wherein n is 1, 2, 3, 4, 5, or 6, and R
³ is C₁-C₂ alkyl optionally substituted by fluoro; phenyl optionally substituted by fluoro; pyridinyl optionally substituted by fluoro or methyl; or cyclopropyl optionally substituted by fluoro; or
wherein in the compound of Formula (II), or a salt thereof, R² is selected from the group consisting of 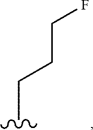 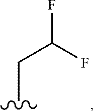 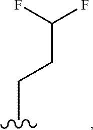 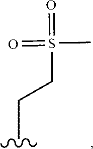 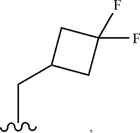 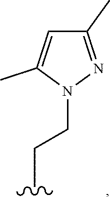 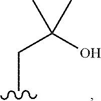 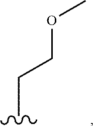 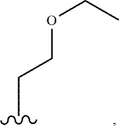 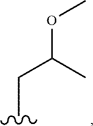 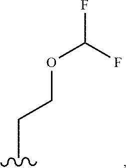 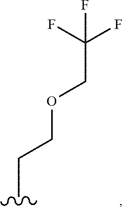 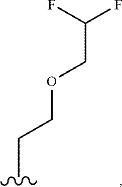 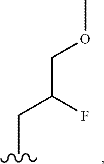 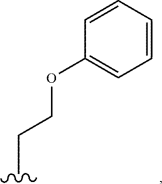 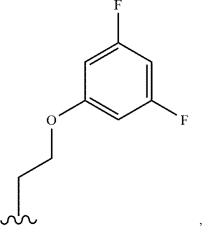 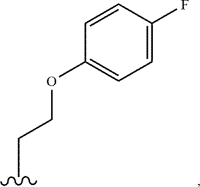 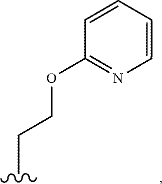 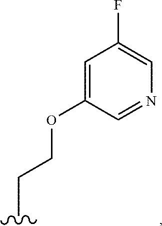 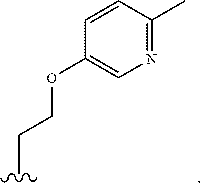 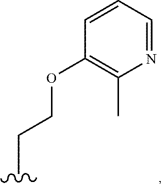 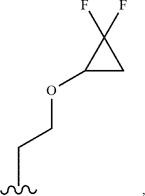 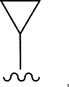 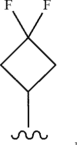 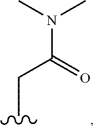 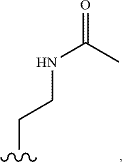
and any of the foregoing groups wherein any one or more hydrogen atom(s) are replaced with deuterium atom(s); or
wherein in the compound of Formula (II), or a salt thereof, R² is selected from the group consisting of 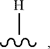 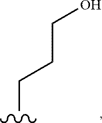 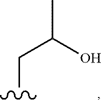 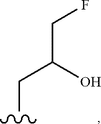 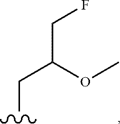 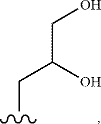 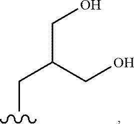 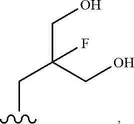 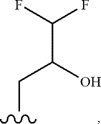 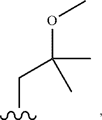 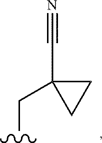 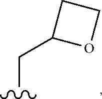 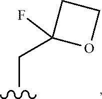 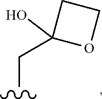 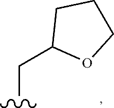 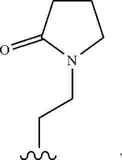 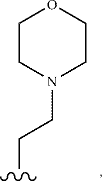 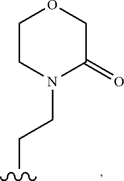 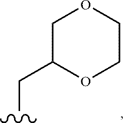 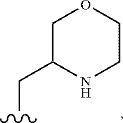 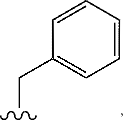 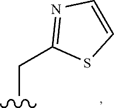 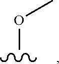
and any of the foregoing groups wherein any one or more hydrogen atom(s) are replaced with deuterium atom(s); or
wherein in the compound of Formula (II), or a salt thereof, R² is C₃-C₅ alkyl substituted by both fluorine and -OCH₃ ; or
wherein in the compound of Formula (II), or a salt thereof, R² is C₁-C₆ alkyl optionally substituted by -OR³, and R³ is phenyl optionally substituted by fluorine; or
wherein in the compound of Formula (II), or a salt thereof, R² is C₁-C₆ alkyl optionally substituted by -OR³, and R³ is pyridinyl optionally substituted by fluorine or methyl; or
wherein in the compound of Formula (II), or a salt thereof, R² is C₁-C₆ alkyl substituted by R^{2a} wherein R^{2a} is halogen; or
wherein in the compound of Formula (II), or a salt thereof, R² is C₁-C₆ alkyl substituted by R^{2a} wherein R^{2a} is deuterium; or
wherein in the compound of Formula (II), or a salt thereof, R² is C₁-C₆ alkyl substituted by R^{2a} wherein R^{2a} is 3- to 12-membered heterocyclyl optionally substituted by oxo; or wherein in the compound of Formula (II), or a salt thereof, R² is C₁-C₆ alkyl substituted by R^{2a} wherein R^{2a} is 4- to 5-membered heterocyclyl optionally substituted by oxo; or
wherein in the compound of Formula (II), or a salt thereof, R² is C₁-C₆ alkyl substituted by R^{2a} wherein R^{2a} is C₆-C₁₄ aryl optionally substituted by halogen or -OR⁶ ; or
wherein in the compound of Formula (II), or a salt thereof, R² is C₁-C₆ alkyl substituted by R^{2a} wherein R^{2a} is phenyl optionally substituted by halogen or -OR⁶ ; or
wherein in the compound of Formula (II), or a salt thereof, R² is C₁-C₆ alkyl substituted by R^{2a} wherein R^{2a} is 5- to 10-membered heteroaryl optionally substituted by C₁-C₆ alkyl; or
wherein in the compound of Formula (II), or a salt thereof, R² is C₁-C₆ alkyl substituted by R^{2a} wherein R^{2a} is pyrazolyl optionally substituted by methyl; or
wherein in the compound of Formula (II), or a salt thereof, R² is C₁-C₆ alkyl substituted by R^{2a} wherein R^{2a} is C₃-C₈ cycloalkyl optionally substituted by —CN, halogen, or -OR⁶ ; or
wherein in the compound of Formula (II), or a salt thereof, R² is C₁-C₆ alkyl substituted by R^{2a} wherein R^{2a} is -S(O)₂R³ ; or
wherein in the compound of Formula (II), or a salt thereof, R¹ is pyridyl optionally substituted by R^{1a} ; or
wherein in the compound of Formula (II), or a salt thereof, R¹ is indazolyl optionally substituted by R^{1a} ; or
wherein in the compound of Formula (II), or a salt thereof, R¹ is 1H-pyrrolopyridyl optionally substituted by R^{1a} ; or
wherein in the compound of Formula (II), or a salt thereof, R¹ is quinolinyl optionally substituted by R^{1a} ; or
wherein in the compound of Formula (II), or a salt thereof, R¹ is phenyl optionally substituted by R^{1a} ; or
wherein in the compound of Formula (II), or a salt thereof, R¹ is indanyl optionally substituted by R^{1a}.

12. The method of claim 1, wherein the compound of Formula (II), or a salt thereof, is selected from Compound Nos. 1-66 in FIG. 1.

13. The method of claim 1, wherein the compound of Formula (II), or a salt thereof, is selected from Compound Nos. 1-147.

14. The method of claim 1, wherein the compound of Formula (II), or a salt thereof, is selected from Compound Nos. 1-665.

15. The method of claim 1, wherein the compound of Formula (II), or a salt thereof, is selected from Compound Nos. 1-780.

16. The method of claim 1, wherein the compound of Formula (II), or a salt thereof, is (S)-4-((2-methoxyethyl)(4-(5,6,7,8-tetrahydro-1,8-naphthyridin-2-yl)butyl)amino)-2-(quinazolin-4-ylamino)butanoic acid: 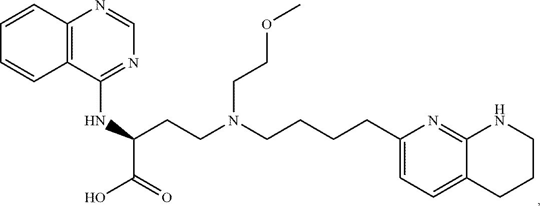
or a salt thereof.

17. The method of claim 1, comprising administering the compound of Formula (II), or a salt thereof, in an amount in milligrams of about 1, 2.5, 5, 7.5, 10, 15, 20, 25, 30, 35, 40, 50, 75, 80, 85, 90, 95, 100, 105, 110, 115, 120, 125, 150, 160, 175, 200, 225, 250, 320, 400, 480, 560, 640, 720, 800, 880, 960, or 1040, or a range between any two of the preceding values.

18. The method of claim 1, comprising administering the compound of Formula (II), or a salt thereof, in an amount effective on administration to the subject to produce a Cₘₐₓ in plasma of the subject in ng/mL of at least about one of 700, 750, 800, 850, 900, 950, 1000, 1050, 1100, 1150, 1200, 1250, 1300, 1350, 1400, 1450, 1500, 1600, 1700, 1800, 1900, 2000, 2100, 2200, 2300, 2400, or 2500, or a range between any two of the preceding concentrations.

19. The method of claim 1, comprising administering the compound of Formula (II), or a salt thereof, in an amount effective on administration to the subject to produce a Cₘₐₓ in plasma of the subject in ng/mL, the Cₘₐₓ corresponding to a plasma-adjusted concentration effective to inhibit a percentage of αvβ₆ or αvβ₁ in the individual of at least about one of 50, 55, 60, 65, 70, 75, 80, 85, 90, 95, or 100, or a range between any two of the preceding percentages.

20. The method of claim 1, comprising administering the compound of Formula (II), or a salt thereof, daily to the subject.

21. The method of claim 1, comprising administering the compound of Formula (II), or a salt thereof, once daily to the subject.

22. The method of claim 1, wherein the daily administering is given one time, two times, three times, or four times daily.

23. The method of claim 1, wherein the daily administering is given once daily.

24. The method of claim 1, wherein the disease is a pulmonary disease.

25. The method of claim 1, wherein the disease is a fibrotic disease.

26. The method of claim 1, wherein the disease is a pulmonary fibrotic disease.

27. The method of claim 1, wherein the disease is selected from the group consisting of: idiopathic pulmonary fibrosis, an interstitial lung disease, radiation-induced pulmonary fibrosis, systemic scleroderma or systemic sclerosis associated interstitial lung disease, and nonspecific interstitial pneumonia.

28. The method of claim 1, wherein the disease is idiopathic pulmonary fibrosis.

29. The method of claim 1, wherein the second drug is pirfenidone, represented by: 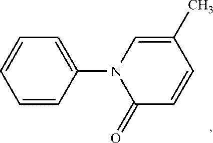
or a salt thereof; or the systematic chemical name 5-methyl-1phenyl-2-1(H)-pyridone, or a salt thereof.

30. The method of claim 29, wherein the pirfenidone or a salt thereof is orally administered.

31. The method of claim 30, wherein the pirfenidone or a salt thereof is orally administered to the subject via at least one of a capsule dosage form and a tablet dosage form.

32. The method of claim 30, wherein the pirfenidone or a salt thereof is orally administered to the subject via the capsule dosage form.

33. The method of claim 32, wherein the capsule dosage form comprises the pirfenidone or a salt thereof and 1, 2, 3, or 4 ingredients selected from the group consisting of: microcrystalline cellulose, croscarmellose sodium, povidone, and magnesium stearate.

34. The method of claim 32, wherein at least one of:
the capsule dosage form is characterized by an amount per capsule of the pirfenidone of one of, or about one of: 267 mg, 534 mg, or 801 mg, or a range between any two of the preceding values; or
the amount of pirfenidone orally administered to the subject via the capsule dosage form in a single administration event is one of, or about one of: 267 mg, 534 mg, or 801 mg, or a range between any two of the preceding values.

35. The method of claim 32, wherein a capsule shell of the capsule dosage form comprises gelatin and titanium dioxide.

36. The method of claim 30, wherein the pirfenidone or a salt thereof pirfenidone or a salt thereof is orally administered to the subject via the tablet dosage form.

37. The method of claim 36, wherein the tablet dosage form comprises the pirfenidone or a salt thereof and 1, 2, 3, 4, 5, 6, 7, 8, 9, or 10 ingredients selected from the group consisting of: Microcrystalline cellulose, colloidal anhydrous silica, povidone, croscarmellose sodium, magnesium stearate, polyvinyl alcohol, titanium dioxide, macrogol (polyethylene glycol), talc, and iron oxide.

38. The method of claim 36, wherein at least one of:
the tablet dosage form is characterized by an amount per capsule of the pirfenidone of one of, or about one of: 267 mg, 534 mg, or 801 mg, or a range between any two of the preceding values; or
the amount of pirfenidone orally administered to the subject via the tablet dosage form in a single administration event is one of, or about one of: 267 mg, 534 mg, or 801 mg, or a range between any two of the preceding values.

39. The method of claim 36, wherein the tablet dosage form comprises an outer coating.

40. The method of claim 30, wherein upon initiation of treatment with the pirfenidone or a salt thereof, the pirfenidone is titrated to a full daily dosage over a period of time.

41. The method of claim 40, wherein upon initiation of treatment with the pirfenidone or a salt thereof, the pirfenidone is titrated to a full daily dosage over a 14-day period as follows: days 1 through 7, 267 mg three times daily to achieve a daily pirfenidone dosage of 801 mg/day; days 8 through 14, 534 mg three times daily to achieve a daily pirfenidone dosage of 1602 mg/day; and days 15 onward, 801 mg three times daily to achieve the full daily pirfenidonedosage of 2403 mg/day.

42. The method of claim 30, wherein the pirfenidone or a salt thereof is administered in a full daily pirfenidone dosage of 2403 mg/day.

43. The method of claim 30, wherein the disease is idiopathic pulmonary fibrosis.

44. The method of claim 30, wherein the pirfenidone is administered as a granulate formulation of 5-methyl-1-phenyl-2-(1H)-pyridone, characterized by one of:
5-methyl-1-phenyl-2-(1H)-pyridone and pharmaceutically acceptable excipients, said excipients comprising an effective amount of binder to increase the AUC of the 5-methyl-1-phenyl-2-(1H)-pyridone at least 45% upon oral administration, as compared to pirfenidone without excipients orally administered in a capsule shell; or
granules comprising 5-methyl-1-phenyl-2-(1H)-pyridone and a glidant, and one or more extragranular excipients comprising an extragranular glidant.

45. The method of claim 30, wherein the pirfenidone is administered as a coated tablet dosage form comprising a compressed tablet comprising 5-methyl-1-phenyl-2-(1H)-pyridone as an active ingredient; and a coating comprising a light shielding agent disposed on the compressed tablet.

46. The method of claim 30, wherein the pirfenidone is administered as a capsule dosage form, wherein the capsule dosage form is characterized by one of:
a capsule comprising a pharmaceutical formulation of 5-methyl-1-phenyl-2-(1H)-pyridone, wherein said pharmaceutical formulation comprises 5-30% by weight of pharmaceutically acceptable excipients and 70-95% by weight of 5-methyl-1-phenyl-2-(1H)-pyridone, wherein said excipients comprise an effective amount of binder to increase the AUC of pirfenidone upon oral administration, as compared to a capsule comprising no excipients;
a capsule comprising a pharmaceutical formulation of 5-methyl-1-phenyl-2-(1H)-pyridone, wherein said pharmaceutical formulation comprises 5-methyl-1-phenyl-2-(1H)-pyridone and pharmaceutically acceptable excipients, said excipients comprising an effective amount of binder to increase the AUC of pirfenidone upon oral administration, as compared to a capsule comprising no excipients;
a capsule comprising a pharmaceutical formulation of 5-methyl-1-phenyl-2-(1H)-pyridone, wherein said pharmaceutical formulation comprises pharmaceutically acceptable excipients and 5-methyl-1-phenyl-2-(1H)-pyridone, and the formulation is stable for at least 9 months at 40° C. at 75% relative humidity, as measured by a dissolution of at least 85% of the 5-methyl-1-phenyl-2-(1H)-pyridone after the at least 9 months; or
a capsule comprising a pharmaceutical formulation of 5 methyl-1-phenyl-2-(1H)-pyridone, wherein said pharmaceutical formulation comprises pharmaceutically acceptable excipients and 5-methyl-1-phenyl-2-(1H)-pyridone, and the formulation is stable for at least 18 months at 25° C. at 60% relative humidity, as measured by a dissolution of at least 93% of the 5-methyl-1-phenyl-2-(1H)-pyridone after the at least 18 months.

47. The method of claim 1, wherein the second drug is nintedanib or a salt thereof, and is represented by one or both of: 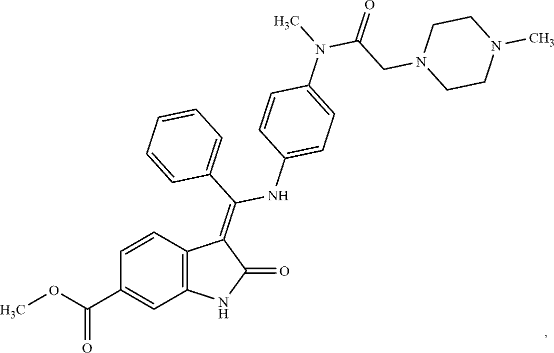
or a salt thereof; or
the systematic chemical name 3-Z-[1-(4-(N-((4-methyl-piperazin-1-yl)-methylcarbonyl)-N-methyl-amino)-anilino)-1-phenyl-methylene]-6-methoxycarbonyl-2-indolinone, or a salt thereof.

48. The method of claim 47, wherein the salt of nintedanib is represented by one or both of: 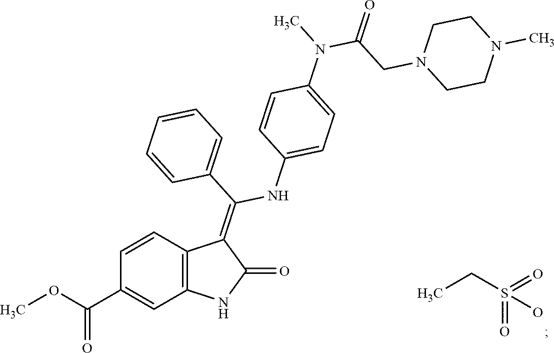
or
the systematic chemical name 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate.

49. The method of claim 47,wherein the the nintedanib or a salt thereof is characterized as one or more of:
3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate in crystalline form, having a melting point of Tm.p.=305±5° C. (determined by DSC; evaluation using peak-maximum; heating rate: 10° C./min);
crystalline 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate according to claim 2, the X-ray powder diagram of which includes, inter alia, the characteristic values d=5.43 Å, 5.08 Å, 4.71 Å, 4.50 Å and 4.43 Å with an intensity of more than 40%;
crystalline 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate according to claim 2, characterised by a unit cell determined by X-ray powder diffractometric measurements having the following dimensions: a=16.332 Å, b=19.199 Å, c=11.503 Å, α=95.27°, β=90.13°, γ=110.83° and V=3354.4 Å3;
a pharmaceutical composition comprising 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate and one or more inert carriers and/or diluents;
a prodrug of 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate; or
3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate hemihydrate in crystalline form.

50. The method of claim 47, wherein the nintedanib or salt thereof is orally administered.

51. The method of claim 47, wherein the nintedanib or a salt thereof is orally administered to the subject via at least one of a lipid dosage form and a capsule dosage form.

52. The method of claim 51, wherein at least one of:
the lipid dosage form is characterized by an amount of the nintedanib or salt thereof equivalent to, or equivalent to about, 100 mg or 150 mg of nintedanib, or a range between about 100 mg to about 150 mg of nintedanib; or
the amount of nintedanib or salt thereof orally administered to the subject via the lipid dosage form in a single administration event is equivalent to, or equivalent to about, 100 mg or 150 mg of nintedanib, or a range between about 100 mg to about 150 mg of nintedanib.

53. The method of claim 51, wherein at least one of:
the lipid dosage form is characterized by an amount of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib; or
the amount of nintedanib or salt thereof orally administered to the subject via the lipid dosage form in a single administration event is characterized by an amount per capsule of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib.

54. The method of claim 51, wherein the nintedanib or a salt thereof is orally administered to the subject via the lipid dosage form, the lipid dosage form characterized by one or more of:
(a) a formulation of the active substance 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate which comprises a lipid suspension of the active substance in 1 to 90 wt. % of medium chain triglycerides, 1 to 30 wt. % of hard fat and 0.1 to 10 wt. % of lecithin;
(b) a pharmaceutical dosage form which is a viscous lipid suspension formulation comprising: 10 to 50 wt. % of the active substance 3-Z-[1-(4-(N-((4-methyl-piperazin-1-yl)-methylcarbonyl)-N-methyl-amino)-anilino)-1-phenyl-methylenel]-6-methoxycarbonyl-2-indolinone-monoethanesulphonate, 10 to 70 wt. % of medium chain triglycerides; 10 to 30 wt. % of hard fat; and 0.25 to 2.5 wt. % of lecithin, which delivers an immediate release profile in which not less than 70% (Q65%) of the active substance is dissolved in 60 minutes in vitro under the following in vitro dissolution conditions according to European Pharmacopeia 6.2: Apparatus 2 (paddle), dissolution medium with 0.1 M HCl (pH 1) and stirring speed of 50 to 150 rpm, at a temperature of 37° C.; or
(c) a lipid suspension comprising, consisting of, or consisting essentially of 3-Z-[1-(4-(N-((4-methyl-piperazin-1-yl)-methylcarbonyl)-N-methyl-amino)-anilino)-1-phenylmethylene]-6-methoxycarbonyl-2-indolinone-monoethanesulphonate, medium chain triglycerides, hard fat and lecithin, wherein the medium chain triglycerides, hard fat and lecithin are present in the lipid suspension in the following amounts: 1 to 90 wt. % of medium chain triglycerides, 1 to 30 wt. % of hard fat, and 0.1 to 10 wt. % of lecithin.

55. The method of claim 51, wherein the nintedanib or a salt thereof is orally administered to the subject via the capsule dosage form, the capsule dosage form comprising a capsule shell and a capsule formulation.

56. The method of claim 51, wherein the nintedanib or a salt thereof is orally administered to the subject via the capsule dosage form, the capsule dosage form comprising a capsule shell and a capsule formulation, the capsule formulation comprising the lipid dosage form characterized by one or more of:
(a) a formulation of the active substance 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate which comprises a lipid suspension of the active substance in 1 to 90 wt. % of medium chain triglycerides, 1 to 30 wt. % of hard fat and 0.1 to 10 wt. % of lecithin;
(b) a pharmaceutical dosage form which is a viscous lipid suspension formulation comprising: 10 to 50 wt. % of the active substance 3-Z-[1-(4-(N-((4-methyl-piperazin-1-yl)-methylcarbonyl)-N-methyl-amino)-anilino)-1-phenyl-methylenel]-6-methoxycarbonyl-2-indolinone-monoethanesulphonate, 10 to 70 wt. % of medium chain triglycerides; 10 to 30 wt. % of hard fat; and 0.25 to 2.5 wt. % of lecithin, which delivers an immediate release profile in which not less than 70% (Q65%) of the active substance is dissolved in 60 minutes in vitro under the following in vitro dissolution conditions according to European Pharmacopeia 6.2: Apparatus 2 (paddle), dissolution medium with 0.1 M HCl (pH 1) and stirring speed of 50 to 150 rpm, at a temperature of 37° C.; or
(c) a lipid suspension comprising, consisting of, or consisting essentially of 3-Z-[1-(4-(N-((4-methyl-piperazin-1-yl)-methylcarbonyl)-N-methyl-amino)-anilino)-1-phenylmethylene]-6-methoxycarbonyl-2-indolinone-monoethanesulphonate, medium chain triglycerides, hard fat and lecithin, wherein the medium chain triglycerides, hard fat and lecithin are present in the lipid suspension in the following amounts: 1 to 90 wt. % of medium chain triglycerides, 1 to 30 wt. % of hard fat, and 0.1 to 10 wt. % of lecithin.

57. The method of claim 55, wherein at least one of:
the capsule dosage form is characterized by an amount per capsule of the nintedanib or salt thereof equivalent to, or equivalent to about, 100 mg or 150 mg of nintedanib, or a range between about 100 mg to about 150 mg of nintedanib; or.
the amount of nintedanib or salt thereof orally administered to the subject via the capsule dosage form in a single administration event is equivalent to, or equivalent to about, 100 mg or 150 mg of nintedanib, or a range between about 100 mg to about 150 mg of nintedanib.

58. The method of claim 55, wherein at least one of:
the capsule dosage form is characterized by an amount per capsule of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib; or.
the amount of nintedanib or salt thereof orally administered to the subject via the capsule dosage form in a single administration event is characterized by an amount per capsule of nintedanib ethane sulfonate of, or of about 120.40 mg or 180.60 mg, or a range between about 120.40 mg to about 180.60 mg of nintedanib ethane sulfonate, respectively equivalent to, or equivalent to about, 100 mg or 150 mg of nintedanib, or a range between about 100 mg to about 150 mg of nintedanib.

59. The method of claim 55, wherein the capsule shell of the capsule dosage form comprises 1, 2, 3, 4, 5, or 6 of: gelatin, glycerol, titanium dioxide, red ferric oxide, yellow ferric oxide, and black ink.

60. The method of claim 1, wherein the second drug is nintedanib or a salt thereof and is administered to the subject twice daily in a dosage of the nintedanib or a salt thereof equivalent to, or equivalent to about, 100 mg of nintedanib, for a total daily dose equivalent to, or equivalent to about, 200 mg of nintedanib.

61. The method of claim 60, wherein the subject has one of a mild hepatic impairment or a side effect associated with nintedanib or a salt thereof.

62. The method of claim 1, wherein the second drug is nintedanib or a salt thereof and is administered to the subject twice daily in a dosage of the nintedanib or a salt thereof equivalent to, or equivalent to about, 150 mg of nintedanib, for a total daily dose equivalent to, or equivalent to about, 300 mg of nintedanib.

63. The method of claim 47, wherein the disease is selected from the group consisting of idiopathic pulmonary fibrosis, an interstitial lung disease, and systemic sclerosis-associated interstitial lung disease.

64. The method of claim 47, wherein the disease is idiopathic pulmonary fibrosis.

65. The method of claim 63, wherein the interstitial lung disease includes chronic fibrosing interstitial lung diseases (ILDs) with a progressive phenotype.

66. The method of claim 63, wherein the disease includes systemic sclerosis-associated interstitial lung disease, and treating the subject includes slowing the rate of decline in pulmonary function in the subject associated with the systemic sclerosis-associated interstitial lung disease.

67. The method of claim 1, comprising administering the first drug to the subject in an amount effective to modulate at least one integrin in the subject.

68. The method of claim 1, wherein the subject has at least one tissue in need of therapy and the tissue has at least one elevated level of:
at least one integrin activity and/or expression;
a pSMAD/SMAD value;
new collagen formation or accumulation;
total collagen; and
Type I Collagen gene Collal expression;
and wherein the level is elevated compared to a healthy state of the tissue.

69. The method of claim 67, comprising administering the first drug to the subject in an amount effective to inhibit the at least one integrin in the subject.

70. The method of claim 67, wherein the at least one integrin in the subject comprises αv.

71. The method of claim 67, wherein the at least one integrin in the subject is selected from the group consisting of αvβ₆ integrin and αvβ₁ integrin.

72. The method of claim 67, wherein the at least one integrin in the subject comprises both αvβ₆ integrin and αvβ₁ integrin.

73. The method of claim 67, comprising administering the first drug to the subject in an amount effective to inhibit one or both of αvβ₁ integrin and αvβ₆ integrin in the subject.

74. The method of claim 67, wherein the method selectively reduces αvβ₁ integrin activity and/or expression compared to αvβ₆ integrin activity and/or expression in the subject.

75. The method of claim 67, wherein the method selectively reduces αvβ₆ integrin activity and/or expression compared to αvβ₁ integrin activity and/or expression in the subject.

76. The method of claim 67, wherein the method reduces both αvβ₁ integrin and αvβ₆ integrin activity and/or expression compared to at least one other αv-containing integrin in the subject.

77. The method of claim 67, wherein the activity of αvβ₁ integrin in one or more fibroblasts is reduced in the subject.

78. The method of claim 67, wherein the activity of αvβ₆ integrin in one or more epithelial cells is reduced in the subject.

79. The method of claim 68, wherein the at least one tissue in the subject comprises one or more of: lung tissue, liver tissue, skin tissue, cardiac tissue, kidney tissue, gastrointestinal tissue, gall bladder tissue, and bile duct tissue.

80. The method of claim 68, wherein the tissue has an elevated pSMAD2/SMAD2 value or an elevated pSMAD3/SMAD3 value compared to the healthy state of the tissue.

81. The method of claim 1, wherein the first drug and/or the second drug are administered orally to the subject.

82. The method of claim 1 wherein the first drug and/or the second drug are administered to the subject with food.

83. The method of claim 1 wherein the first drug and the second drug are administered to the subject at the same time or on a same schedule.

84. The method of claim 1 wherein the first drug and the second drug are administered to the subject at different times or on a different schedule.

85. The method of claim 1 wherein the second drug is administered to the subject over a period of days, weeks, months, or years before first administering the first drug to the subject.

86. The method of claim 1 wherein after administering the first and second drugs to the subject over a period of days, weeks, months, or years, the dose of the second drug is decreased in amount or frequency.

87. The method of claim 1 wherein after administering the first and second drugs to the subject over a period of days, weeks, months, or years, administration of the second drug is discontinued.

88. The method of claim 86 wherein the second drug is decreased in amount or frequency or discontinued after the subject experiences a stabilization, improvement, or remission in the disease.

89. The method of claim 1 wherein the subject is human.
